SEQUENCE LISTING (585 sequences)

SEQ ID NO: 1 - protein, 866 aa, Escherichia coli
MSTKPLFLLRKAKKSSGEPDVVLWASNDFESTCATLDYLIVKSGKKLSSYFKAVATNFPVVNDLPAEGEIDFTWSERYQLSKDSMTWELKPGAAPDNAHYQGNTNVNGEDMTEIEENMLLPISGQELPIRWLAQHGSEKPVTHVSRDGLQALHIARAEELPAVTALAVSHKTSLLDPLEIRELHKLVRDTDKVFPNPGNSNLGLITAFFEAYLNADYTDRGLLTKEWMKGNRVSHITRTASGANAGGGNLTDRGEGFVHDLTSLARDVATGVLARSMDLDIYNLHPAHAKRIEEIIAENKPPFSVFRDKFITMPGGLDYSRAIVVASVKEAPIGIEVIPAHVTEYLNKVLTETDHANPDPEIVDIACGRSSAPMPQRVTEEGKQDDEEKPQPSGTTAVEQGEAETMEPDATEHHQDTQPLDAQSQVNSVDAKYQELRAELHEARKNIPSKNPVDDDKLLAASRGEFVDGISDPNDPKWVKGIQTRDCVYQNQPETEKTSPDMNQPEPVVQQEPEIACNACGQTGGDNCPDCGAVMGDATYQETFDEESQVEAKENDPEEMEGAEHPHNENAGSDPHRDCSDETGEVADPVIVEDIEPGIYYGISNENYHAGPGISKSQLDDIADTPALYLWRKNAPVDTTKTKTLDLGTAFHCRVLEPEEFSNRFIVAPEFNRRTNAGKEEEKAFLMECASTGKTVITAEEGRKIELMYQSVMALPLGQWLVESAGHAESSIYWEDPETGILCRCRPDKIIPEFHWIMDVKTTADIQRFKTAYYDYRYHVQDAFYSDGYEAQFGVQPTFVFLVASTTIECGRYPVEIFMMGEEAKLAGQQEYHRNLRTLADCLNTDEWPAIKTLSLPRWAKEYAND

SEQ ID NO: 2 - protein, 280 aa, Synthetic construct
ADPVIVEDIEPGIYYGISNENYHAGPGVSKSQLDDIADTPALYLWRKNAPVDTTKTKTLDLGTAFHCRVLEPEEFSNRFIVAPEFNRRTNSGKEEEKAFLRECASTGKTVITAEEGRKIELMYQSVMALPLGQWLVESAGHAESSIYWEDPETAILCRCRPDKIIPEFHWIMDVKTTADIQRFKTAYYDYRYHVQDAFYSDGYEAQFGVQPTFVFLVASTTIECGRYPVEIFMMGEEAKLAGQLEYHRNLRTLADCLNTDEWPAIKTLSLPRWAKEYAND

SEQ ID NO: 3 - protein, 265 aa, Synthetic construct
GISNENYHAGPGVSKSQLDDIADTPALYLWRKNAPVDTTKTKTLDLGTAFHCRVLEPEEFSNRFIVAPEFNRRTNSGKEEEKAFLRECASTGKTVITAEEGRKIELMYQSVMALPLGQWLVESAGHAESSIYWEDPETAILCRCRPDKIIPEFHWIMDVKTTADIQRFKTAYYDYRYHVQDAFYSDGYEAQFGVQPTFVFLVASTTIECGRYPVEIFMMGEEAKLAGQLEYHRNLRTLADCLNTDEWPAIKTLSLPRWAKEYAND

SEQ ID NO: 4 - protein, 272 aa, Pantoea brenneri
MQPGIYYDISNEDYHRGAGISKSQLDDIAISPAIYQWRKHAPVDEEKTAALDLGTALHCLLLEPDEFSKRFQIGPEVNRRTTAGKEKEKEFIERCEAEGITPITHDDNRKLKLMRDSALAHPIARWMLEAQGNAEASIYWNDRDAGVLSRCRPDKIITEFNWCVDVKSTADIMKFQKDFYSYRYHVQDAFYSDGYESHFHETPTFAFLAVSTSIDCGRYPVQVFIMDQQAKDAGRAEYKRNIHTFAECLSRNEWPGIATLSLPFWAKELRNE

SEQ ID NO: 5 - protein, 272 aa, unidentified
  source 1..272: note = Type-F symbiont of Plautia stali
MQPGIYYDISNEDYHGGPGISKSQLDDIAISPAIYQWRKHAPVDEEKTAALDLGTALHCLLLEPDEFSKRFEIGPEVNRRTTAGKEKEKEFMERCEAEGVTPITHDDNRKLRLMRDSAMAHPIARWMLEAQGNAEASIYWNDRDTGVLSRCRPDKIITDFNWCVDVKSTADIIKFQKDFYSYRYHVQDAFYSDGYESHFDETPTFAFLAVSTSIDCGRYPVQVFIMDQQAKDAGRAEYKRNIHTFAECLSRNEWPGIATLSLPYWAKELRNE

SEQ ID NO: 6 - protein, 272 aa, Providencia sp.
MKEGIYYNISNEDYHNGLGISKSQLDLINEMPAEYIWSKEAPVDEEKIKPLEIGTALHCLLLEPDEYHKRYKIGPDVNRRTNVGKEKEKEFFDMCEKEGITPITHDDNRKLMIMRDSALAHPIAKWCLEADGVSESSIYWTDKETDVLCRCRPDRIITAHNYIIDVKSSGDIEKFDYEYYNYRYHVQDAFYSDGYKEVTGITPTFLFLVVSTKIDCGKYPVRTYVMSEEAKSAGRTAYKHNLLTYAECLKTDEWAGIRTLSLPRWAKELRNE

SEQ ID NO: 7 - protein, 648 aa, Shigella sonnei
DRGLLTKEWRKGNRVSRITRTASGANAGGGNLTDRGEGFVHDLTSLARDIATGVLARSMDVDIYNLHPAHAKRIEEIIAENKPPFSVFRDKFITMPGGLDYSRAIVVASVKEAPIGIEVIPAHVTAYLNKVLTETDHANPDPEIVDIACGRSSAPMPQRVTEEGKQDDEEKLQPSGTTADEQGEAETMEPDATKHHQDTQPLDAQSQVNSVDAKYQELRAELHEARKNIPSKNPVDADKLLAASRGEFVDGISDPNDPKWVKGIQTRDSVYQNQPETEKTSPDMKQPEPVVQQEPEIAFNACGQTGGDNCPDCGAVMGDATYQETFDEENQVEAKENDPEEMEGAEHPHNENAGSDPHRDCSDETGEVADPVIVEDIEPGIYYGISNENYHAGPGVSKSQLDDIADTPALYLWRKNAPVDTTKTKTLDLGTAFHCRVLEPEEFSNRFIVAPEFNRRTNAGKEEEKAFLMECASTGKMVITAEEGRKIELMYQSVMALPLGQWLVESAGHAESSIYWEDPETGILCRCRPDKIIPEFHWIMDVKTTADIQRFKTAYYDYRYHVQDAFYSDGYEAQFGVQPTFVFLVASTTIECGRYPVEIFMMGEEAKLAGQLEYHRNLRTLADCLNTDEWPAIKTLSLPRWAKEYAND

SEQ ID NO: 8 - protein, 300 aa, Pseudobacteriovorax antillogorgiicola
MSKLSNLKVSNSDVDTLSRIRMKEGVYRDLPIESYHQSPGYSKTSLCQIDKAPIYLKTKVPQKSTKSLNIGTAFHEAMEGVFKDKYVVHPDPGVNKTTKSWKDFVKRYPKHMPLKRSEYDQVLAMYDAARSYRPFQKYHLSRGFYESSFYWHDAVTNSLIKCRPDYITPDGMSVIDFKTTVDPSPKGFQYQAYKYHYYVSAALTLEGIEAVTGIRPKEYLFLAVSNSAPYLTALYRASEKEIALGDHFIRRSLLTLKTCLESGKWPGLQEEILELGLPFSGLKELREEQEVEDEFMELVG

SEQ ID NO: 9 - protein, 269 aa, Escherichia coli
MTKQPPIAKADLQKTQGNRAPAAVKNSDVISFINQPSMKEQLAAALPRHMTAERMIRIATTEIRKVPALGNCDTMSFVSAIVQCSQLGLEPGSALGHAYLLPFGNKNEKSGKKNVQLIIGYRGMIDLARRSGQIASLSARVVREGDEFSFEFGLDEKLIHRPGENEDAPVTHVYAVARLKDGGTQFEVMTRKQIELVRSLSKAGNNGPWVTHWEEMAKKTAIRRLFKYLPVSIEIQRAVSMDEKEPLTIDPADSSVLTGEYSVIDNSEE

SEQ ID NO: 10 - protein, 273 aa, Pantoea brenneri
MSNQPPIASADLQKTQQSKQVANKTPEQTLVGFMNQPAMKSQLAAALPRHMTADRMIRIVTTEIRKTPQLAQCDQSSFIGAVVQCSQLGLEPGSALGHAYLLPFGNGRSKSGQSNVQLIIGYRGMIDLARRSGQIVSLSARVVRADDEFSFEYGLDENLVHRPGENEDAPITHVYAVARLKDGGTQFEVMTVKQVEKVKAQSKASSNGPWVTHWEEMAKKTVIRRLFKYLPVSIEMQKAVVLDEKAESDVDQDNASVLSAEYSVLESGDEATN

SEQ ID NO: 11 - protein, 271 aa, unidentified
  source 1..271: note = Type-F symbiont of Plautia stali
MSNQPPIASADLQKTQQSKQVANKTPEQTLVGFMNQPAMKSQLAAALPRHMTADRMIRIVTTEIRKTPALATCDQSSFIGAVVQCSQLGLEPGSALGHAYLLPFGNGRSKSGQSNVQLIIGYRGMIDLARRSGQIVSLSARVVRADDEFSFEYGLDENLIHRPGDNEDAPITHVYAVARLKDGGTQFEVMTAKQVEKVKAQSKASSNGPWVTHWEEMAKKTVIRRLFKYLPVSIEMQKAVVLDEKAESDVDQDNASVLSAEYSVLEGDGGE

SEQ ID NO: 12 - protein, 268 aa, Providencia sp.
MSNPPLAQSDLQKTQGTEVKVKTKDQQLIQFINQPSMKAQLAAALPRHMTPDRMIRIVTTEIRKTPALATCDMQSFVGAVVQCSQLGLEPGNALGHAYLLPFGNGKAKSGQSNVQLIIGYRGMIDLARRSNQIISISARTVRQGDNFHFEYGLNEDLTHTPSENEDSPITHVYAVARLKDGGVQFEVMTYNQVEKVRASSKAGQNGPWVSHWEEMAKKTVIRRLFKYLPVSIEMQKAVVLDEKAEANVDQENATIFEGEYEEVGTDGN

SEQ ID NO: 13 - protein, 269 aa, Shigella sonnei
MTKQPPIAKADLQKTQENRAPAAIKNNDVISFINQPSMKEQLAAALPRHMTAERMIRIATTEIRKVPALGNCDTMSFVSAIVQCSQLGLEPGSALGHAYLLPFGNKNEKSGKKNVQLIIGYRGMIDLARRSGQIASLSARVVREGDEFNFEFGLDEKLIHRPGENEDAPVTHVYAVARLKDGGTQFEVMTRRQIELVRSQSKAGNNGPWVTHWEEMAKKTAIRRLFKYLPVSIEIQRAVSMDEKEPLTIDPADSSVLTGEYSVIDNSEE

SEQ ID NO: 14 - protein, 242 aa, Pseudobacteriovorax antillogorgiicola
MGHLVSKTEQDYIKQHYAKGATDQEFEHFIGVCRARGLNPAANQIYFVKYRSKDGPAKPAFILSIDSLRLIAHRTGDYAGCSEPIFTDGGKACTVTVRRNLKSGETGNFSGMAFYDEQVQQKNGRPTSFWQSKPRTMLEKCAEAKALRKAFPQDLGQFYIREEMPPQYDEPIQVHKPKALEEPRFSKSDLSRRKGLNRKLSALGVDPSRFDEVATFLDGTPDRELGQKLKLWLKEAGYGVNQ

SEQ ID NO: 15 - protein, 16 aa, Synthetic construct
SGSETPGTSESATPES

SEQ ID NO: 16 - protein, 7 aa, Synthetic construct
PKKKRKV

SEQ ID NO: 17 - protein, 39 aa, Synthetic construct
NSKKRSLEDNETEIKVSRDTWNTKNMRSLEPPRSKKRIH

SEQ ID NO: 18 - protein, 9 aa, Synthetic construct
PAAKRVKLD

SEQ ID NO: 19 - protein, 18 aa, Synthetic construct
KRTADGSEFESPKKKRKV

SEQ ID NO: 20 - protein, 29 aa, Synthetic construct
PEKKRRRPSGSVPVLARPSPPKAGKSSCI

SEQ ID NO: 21 - DNA, 20 bp, Synthetic construct
  source 1..20: mol_type = other DNA
gtcacctccaatgactaggg

SEQ ID NO: 22 - DNA, 20 bp, Synthetic construct
  source 1..20: mol_type = other DNA
ggtgagtgagtgtgtgcgtg

SEQ ID NO: 23 - DNA, 20 bp, Synthetic construct
  source 1..20: mol_type = other DNA
aaggccataggctggactgc

SEQ ID NO: 24 - DNA, 20 bp, Synthetic construct
  source 1..20: mol_type = other DNA
gtagactaatctctggctga

SEQ ID NO: 25 - DNA, 20 bp, Synthetic construct
  source 1..20: mol_type = other DNA
tctcccatgcattcaaactg

SEQ ID NO: 26 - DNA, 20 bp, Synthetic construct
  source 1..20: mol_type = other DNA
accccacagtggggccacta

SEQ ID NO: 27 - DNA, 20 bp, Synthetic construct
  source 1..20: mol_type = other DNA
gtcacctccaatgactaggg

SEQ ID NO: 28 - DNA, 20 bp, Synthetic construct
  source 1..20: mol_type = other DNA
gtcacctccaatgactaggg

SEQ ID NO: 29 - DNA, 20 bp, Synthetic construct
  source 1..20: mol_type = other DNA
aaggccataggctggactgc

SEQ ID NO: 30 - DNA, 20 bp, Synthetic construct
  source 1..20: mol_type = other DNA
ggcactgacgatgcagtaca

SEQ ID NO: 31 - DNA, 20 bp, Synthetic construct
  source 1..20: mol_type = other DNA
gtagactaatctctggctga

SEQ ID NO: 32 - DNA, 20 bp, Synthetic construct
  source 1..20: mol_type = other DNA
tcgtcatctccttcaagggg

SEQ ID NO: 33 - DNA, 20 bp, Synthetic construct
  source 1..20: mol_type = other DNA
atgcatgggagagcccagag

SEQ ID NO: 34 - DNA, 20 bp, Synthetic construct
  source 1..20: mol_type = other DNA
gcctgcccttctaggaatgg

SEQ ID NO: 35 - DNA, 28 bp, Synthetic construct
  source 1..28: mol_type = other DNA
cattctgcctctctgtatggaaaagagc

SEQ ID NO: 36 - DNA, 26 bp, Synthetic construct
  source 1..26: mol_type = other DNA
cccattgaactacctgggcctgattc

SEQ ID NO: 37 - DNA, 22 bp, Synthetic construct
  source 1..22: mol_type = other DNA
aggtttgaatcatcacgcaggc

SEQ ID NO: 38 - DNA, 22 bp, Synthetic construct
  source 1..22: mol_type = other DNA
attcaagtggggaatggcaagc

SEQ ID NO: 39 - DNA, 20 bp, Synthetic construct
  source 1..20: mol_type = other DNA
tgccgtaaatgctgctctct

SEQ ID NO: 40 - DNA, 22 bp, Synthetic construct
  source 1..22: mol_type = other DNA
agacttgccaaggttctttgtg

SEQ ID NO: 41 - DNA, 25 bp, Synthetic construct
  source 1..25: mol_type = other DNA
agtgacctgtgtaattatgcagaag

SEQ ID NO: 42 - DNA, 24 bp, Synthetic construct
  source 1..24: mol_type = other DNA
tgaaagtgccacaaaacaaagaga

SEQ ID NO: 43 - DNA, 19 bp, Synthetic construct
  source 1..19: mol_type = other DNA
aagacaagtggcaacgcag

SEQ ID NO: 44 - DNA, 31 bp, Synthetic construct
  source 1..31: mol_type = other DNA
cgtttatgatactatgcagactatgaagaac

SEQ ID NO: 45 - DNA, 23 bp, Synthetic construct
  source 1..23: mol_type = other DNA
atgaagatgaccctactgctgat

SEQ ID NO: 46 - DNA, 26 bp, Synthetic construct
  source 1..26: mol_type = other DNA
tactgtcttgaaagcagatagaaacc

SEQ ID NO: 47 - DNA, 20 bp, Synthetic construct
  source 1..20: mol_type = other DNA
gcagcaaagaaacacctgga

SEQ ID NO: 48 - DNA, 24 bp, Synthetic construct
  source 1..24: mol_type = other DNA
gttgtcatgccatacagacttttt

SEQ ID NO: 49 - DNA, 24 bp, Synthetic construct
  source 1..24: mol_type = other DNA
agcattactagctctgctttagtg

SEQ ID NO: 50 - DNA, 20 bp, Synthetic construct
  source 1..20: mol_type = other DNA
tccacaagactgggtctgag

SEQ ID NO: 51 - DNA, 21 bp, Synthetic construct
  source 1..21: mol_type = other DNA
gcgactatgcacaacgagagg

SEQ ID NO: 52 - DNA, 26 bp, Synthetic construct
  source 1..26: mol_type = other DNA
aagtgtgtctatctactgtgtcccag

SEQ ID NO: 53 - DNA, 20 bp, Synthetic construct
  source 1..20: mol_type = other DNA
gatgctctttccggagcact

SEQ ID NO: 54 - DNA, 21 bp, Synthetic construct
  source 1..21: mol_type = other DNA
gccaaggactcaaacccagaa

SEQ ID NO: 55 - DNA, 21 bp, Synthetic construct
  source 1..21: mol_type = other DNA
tggtggatttcggactaccct

SEQ ID NO: 56 - DNA, 20 bp, Synthetic construct
  source 1..20: mol_type = other DNA
ttcggactggaaccgtcagc

SEQ ID NO: 57 - DNA, 20 bp, Synthetic construct
  source 1..20: mol_type = other DNA
agacgttccttagtgctggc

SEQ ID NO: 58 - DNA, 20 bp, Synthetic construct
  source 1..20: mol_type = other DNA
aaaagtttcagtgcgacgcc

SEQ ID NO: 59 - DNA, 20 bp, Synthetic construct
  source 1..20: mol_type = other DNA
aggaggtcccatcagatgct

SEQ ID NO: 60 - DNA, 20 bp, Synthetic construct
  source 1..20: mol_type = other DNA
ggctggacagcaaacatgga

SEQ ID NO: 61 - DNA, 20 bp, Synthetic construct
  source 1..20: mol_type = other DNA
ggctggacagcaaacatgga

SEQ ID NO: 62 - DNA, 21 bp, Synthetic construct
  source 1..21: mol_type = other DNA
ttgctgccgtacatgaagctg

SEQ ID NO: 63 - DNA, 43 bp, Synthetic construct
  source 1..43: mol_type = other DNA
ccatctcatccctgcgtgtctccagaagaagggctcccatcac

SEQ ID NO: 64 - DNA, 43 bp, Synthetic construct
  source 1..43: mol_type = other DNA
cctctctatgggcagtcggtgatgagcagcaagcagcactctg

SEQ ID NO: 65 - DNA, 43 bp, Synthetic construct
  source 1..43: mol_type = other DNA
ccatctcatccctgcgtgtctcccagcgtcttcgagagtgagg

SEQ ID NO: 66 - DNA, 44 bp, Synthetic construct
  source 1..44: mol_type = other DNA
cctctctatgggcagtcggtgatgttggaatcctggagtgaccc

SEQ ID NO: 67 - DNA, 45 bp, Synthetic construct
  source 1..45: mol_type = other DNA
ccatctcatccctgcgtgtctccacaaaagctccacatgctagga

SEQ ID NO: 68 - DNA, 44 bp, Synthetic construct
  source 1..44: mol_type = other DNA
cctctctatgggcagtcggtgatggctgactttgggctccttct

SEQ ID NO: 69 - DNA, 43 bp, Synthetic construct
  source 1..43: mol_type = other DNA
ccatctcatccctgcgtgtctccacacactccccaggatctca

SEQ ID NO: 70 - DNA, 44 bp, Synthetic construct
  source 1..44: mol_type = other DNA
cctctctatgggcagtcggtgatgaatgtcagctgaagcaggct

SEQ ID NO: 71 - DNA, 43 bp, Synthetic construct
  source 1..43: mol_type = other DNA
ccatctcatccctgcgtgtctccggctaccctgacaactgctt

SEQ ID NO: 72 - DNA, 44 bp, Synthetic construct
  source 1..44: mol_type = other DNA
cctctctatgggcagtcggtgatgaggacagacatgacaaggca

SEQ ID NO: 73 - DNA, 43 bp, Synthetic construct
  source 1..43: mol_type = other DNA
ccatctcatccctgcgtgtctccgcaggcaagctgtcaagggt

SEQ ID NO: 74 - DNA, 44 bp, Synthetic construct
  source 1..44: mol_type = other DNA
cctctctatgggcagtcggtgatgccctcacacccacaccctca

SEQ ID NO: 75 - DNA, 43 bp, Synthetic construct
  source 1..43: mol_type = other DNA
ccatctcatccctgcgtgtctccggaggggtgtcatcgttctg

SEQ ID NO: 76 - DNA, 44 bp, Synthetic construct
  source 1..44: mol_type = other DNA
cctctctatgggcagtcggtgatgcaaattgcgccatagctggg

SEQ ID NO: 77 - DNA, 43 bp, Synthetic construct
  source 1..43: mol_type = other DNA
ccatctcatccctgcgtgtctcctgagcgctcttcgtctttcc

SEQ ID NO: 78 - DNA, 45 bp, Synthetic construct
  source 1..45: mol_type = other DNA
cctctctatgggcagtcggtgatggccaggaacacaggaatgcta

SEQ ID NO: 79 - DNA, 1400 bp, Synthetic construct
  source 1..1400: mol_type = other DNA
cattctgcctctctgtatggaaaagagcatggggctggcccgtggggtggtgtccactttaggccctgtgggagatcatgggaacccacgcagtgggtcataggctctctcatttactactcacatccactctgtgaagaagcgattatgatctctcctctagaaactcgtagagtcccatgtctgccggcttccagagcctgcactcctccaccttggcttggctttgctggggctagaggagctaggatgcacagcagctctgtgaccctttgtttgagaggaacaggaaaaccacccttctctctggcccactgtgtcctcttcctgccctgccatccccttctgtgaatgttagacccatgggagcagctggtcagaggggaccccggcctggggcccctaaccctatgtagcctcagtcttcccatcaggctctcagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactcggatgtacacggtctgcaaccacaaacccacgagggcagagtgctgcttgctgctggccaggcccctgcgtgggcccaagctggactctggccactccctggccaggctttggggaggcctggagtcatggccccacagggcttgaagcccggggccgccattgacagagggacaagcaatgggctggctgaggcctgggaccacttggccttctcctcggagagcctgcctgcctgggcgggcccgcccgccaccgcagcctcccagctgctctccgtgtctccaatctcccttttgttttgatgcatttctgttttaatttattttccaggcaccactgtagtttagtgatccccagtgtcccccttccctatgggaataataaaagtctctctcttaatgacacgggcatccagctccagccccagagcctggggtggtagattccggctctgagggccagtgggggctggtagagcaaacgcgttcagggcctgggagcctggggtggggtactggtggagggggtcaagggtaattcattaactcctctcttttgttgggggaccctggtctctacctccagctccacagcaggagaaacaggctagacatagggaagggccatcctgtatcttgagggaggacaggcccaggtctttcttaacgtattgagaggtgggaatcaggcccaggtagttcaatggg

SEQ ID NO: 80 - DNA, 1390 bp, Synthetic construct
  source 1..1390: mol_type = other DNA
aggtttgaatcatcacgcaggccctggcctccacccgcccccaccagccccctggcctcagttccctggcaacatctggggttgggggggcagcaggaacaagggcctctgtctgcccagctgcctccccctttgggttttgccagactccacagtgcatacgtgggctccaacaggtcctcttccctcccagtcactgactaaccccggaaccacacagcttcccgttctcagctccacaaacttggtgccaaattcttctcccctgggaagcatccctggacacttcccaaaggaccccagtcactccagcctgttggctgccgctcactttgatgtctgcaggccagatgagggctccagatggcacattgtcagagggacacactgtggcccctgtgcccagccctgggctctctgtacatgaagcaactccagtcccaaatatgtagctgtttgggaggtcagaaatagggggtccaggagcaaactccccccaccccctttccaaagcccattccctctttagccagagccggggtgtgcagacggcagtcactagggggcgctcggccaccacagggaagctgggtgaatggagcgagcagcgtcttcgagagtgaggacgtgtgtgtctgtgtgggtgagtgagtgtgcgcactctagaggtgtcgtgttgagggcgttggagcggggagaaggccaggggtcactccaggattccaatagatctgtgtgtccctctccccacccgtccctgtccggctctccgccttcccctgcccccttcaatattcctagcaaagagggaacggctctcaggccctgtccgcacgtaacctcactttcctgctccctcctcgccaatgccccgcgggcgcgtgtctctggacagagtttccgggggcggatgggtaattttcaggctgtgaaccttggtgggggtcgagcttccccttcattgcggcgggctgcgggccaggcttcactgagcgtccgcagagcccgggcccgagccgcgtgtggaagggctgaggctcgcctgtccccgccccccggggcgggccgggggcggggtcccggcggggcggagccatgcgccccccccttttttttttaaaagtcggctggtagcggggaggatcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaat

SEQ ID NO: 81 - DNA, 1551 bp, Synthetic construct
  source 1..1551: mol_type = other DNA
agtgacctgtgtaattatgcagaagaatggagctggattacacacagcaagttcctgcttctgggacagctctactgacggtatgattttcattcatgtttgtgaagttttgttgtgtgaaatatatgactggaagtttcctatctttgaatgcaatgcatgtttatcaccttttaaaacatttaataatagacttgccaaggttctttgtgtagcatagagatgggtacttgaatgttggccttattgtgagtaaaacgtcgtcccccagctttccctgccgtaaatgctgctctcttccctcccgcagggagctgcactgtgcgatgggagaataagaccatgtactgcatcgtcagtgccttcggactgtctattggaagcggagctactaacttcagcctgctgaagcaggctggagacgtggaggagaaccctggacctgccaccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattcctaaccagctgtcctgcctatggcctttctccttttgtctctagttcatcctctaaccaccagccatgaattcagtgaactcttttctcattctctttgttttgtggcactttcacaatgtagaggaaaaaaccaaatgaccgcactgtgatgtgaatggcaccgaagtcagatgagtatccctgtaggtcacctgcagcctgcgttgccacttgtcttaactctgaatatttcatttcaaaggtgctaaaatctgaaatctgctagtgtgaaacttgctctactctctgaaatgattcaaatacactaattttccatactttatacttttgttagaataaattattcaaatctaaagtctgttgtgttcttcatagtctgcatagtatcataaacg

SEQ ID NO: 82 - DNA, 1973 bp, Synthetic construct
  source 1..1973: mol_type = other DNA
gcagcaaagaaacacctggagataaaccctgaccattccattattgagaccttaaggcaaaaggcagaggctgataagaacgacaagtctgtgaaggatctggtcatcttgctttatgaaactgcgctcctgtcttctggcttcagtctggaagatccccagacacatgctaacaggatctacaggatgatcaaacttggtctgggtaagccttatactatgtaatgttaaaaagaaaataaacacacgtgacattgaagaaaatggtgaactttcagttatccaaacttggagcaccttgtcctgcttgctgcttggaggtattaaagtatgttttttttagggataagtaaggtcttacaagagcaaagaaatgaaattgagactcatatgtcctgtaatactgtcttgaaagcagatagaaaccaagagtattaccctaatagctggctttaagaaatctttgtaatatgaggattttattttggaaacaggtattgatgaagatgaccctactgctgatgataccagtgctgctgtaactgaagaaatgccaccccttgaaggagatgacgacacatcacgcatggaagaagtagacggaagcggagctactaacttcagcctgctgaagcaggctggagacgtggaggagaaccctggacctgtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattcctaaatctgtggctgagggatgacttacctgttcagtactctacaattcctctgataatatattttcaaggatgtttttctttatttttgttaatattaaaaagtctgtatggcatgacaactactttaaggggaagataagatttctgtctactaagtgatgctgtgataccttaggcactaaagcagagctagtaatgctttttgagtttcatgttggtttattttcacagattggggtaacgtgcactgtaagacgtatgtaacatgatgttaactttgtggtctaaagtgtttagctgtcaagccggatgcctaagtagaccaaatcttgttattgaagtgttctgagctgtatcttgatgtttagaaaagtattcgttacatcttgtaggatctactttttgaacttttcattccctgtagttgacaattctgcatgtactagtcctctagaaataggttaaactgaagcaacttgatggaaggatctctccacagggcttgttttccaaagaaaagtattgtttggaggagcaaagttaaaagcctacctaagcatatcgtaaagctgttcaaaaataactcagacccagtcttgtgga

SEQ ID NO: 83 - DNA, 2193 bp, Synthetic construct
  source 1..2193: mol_type = other DNA
gatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcaagcttctgacctcttctcttcctcccacagggcctcgagagatctggcagcggaggaagcggagctactaacttcagcctgctgaagcaggctggagacgtggaggagaaccctggacctgtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattcctaaactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggc

SEQ ID NO: 84 - DNA, 1245 bp, Synthetic construct
  source 1..1245: mol_type = other DNA
gcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaatggaagcggagctactaacttcagcctgctgaagcaggctggagacgtggaggagaaccctggacctgccaccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattcctaatgactaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacactt

SEQ ID NO: 85 - DNA, 810 bp, Pantoea stewartii
  source 1..810: mol_type = genomic DNA
agcaaccagccccctatcgcctccgccgatctgcagaaggccaacaccggcaagcaggtggccaataagacccctgagcagacactggtgggcttcatgaatcagccagcaatgaagagccagctggccgccgccctgccaaggcacatgacagccgatcggatgatcagaatcgtgaccacagagatccgcaagacccccgccctggccacatgcgaccagagctccttcatcggcgccgtggtgcagtgttctcagctgggcctggagcctggcagcgccctgggccacgcctacctgctgccatttggcaacggccggagcaagtccggacagtccaatgtgcagctgatcatcggctatagaggcatgatcgatctggcccggagatctggccagatcgtgtctctgagcgccagggtggtgcgcgcagacgatgagttctcctttgagtacggcctggatgagaacctgatccaccggccaggcgagaatgaggacgcacccatcacccacgtgtatgcagtggcaagactgaaggacggaggcacccagttcgaagtgatgacagtgaagcagatcgagaaggtgaaggcccagtccaaggcctctagcaacggaccctgggtgacccactgggaggagatggccaagaaaaccgtgatcaggcgcctgtttaagtacctgcccgtgagcatcgagatgcagaaggccgtgatcctggatgagaaggccgagtctgacgtggatcaggacaatgcctccgtgctgtctgccgagtatagcgtgctggacggctcctctgaggag

SEQ ID NO: 86 - DNA, 813 bp, Pantoea stewartii
  source 1..813: mol_type = genomic DNA
cagcccggcgtgtactatgacatctccaacgaggagtatcacgccggccctggcatcagcaagtcccagctggacgacatcgccgtgtccccagccatcttccagtggagaaagtctgcccccgtggacgatgagaaaaccgccgccctggacctgggcacagccctgcactgcctgctgctggagcctgatgagttctccaagaggtttatgatcggcccagaggtgaaccggagaaccaatgccggcaagcagaaggagcaggacttcctggatatgtgcgagcagcagggcatcacccctatcacacacgacgataaccggaagctgagactgatgagggactctgcctttgcccacccagtggccagatggatgctggagacagagggcaaggccgaggcctctatctactggaatgacagggatacacagatcctgagcaggtgccgccccgacaagctgatcaccgagttctcttggtgcgtggacgtgaagagcacagccgacatcggcaagttccagaaggacttctacagctatcgctaccacgtgcaggacgccttctattccgatggctacgaggcccagttttgcgaggtgccaaccttcgcctttctggtggtgagctcctctatcgattgtggccggtatcccgtgcaggtgtttatcatggaccagcaggcaaaggatgcaggaagggccgagtataagcggaacctgaccacatacgccgagtgccaggcaaggaatgagtggcctggcatcgccacactgagcctgccttactgggccaaggagatccggaatgtg

SEQ ID NO: 87 - DNA, 816 bp, Pantoea brenneri
  source 1..816: mol_type = genomic DNA
agcaaccagccccctatcgcctccgccgatctgcagaaaacccagcagtccaagcaggtggccaacaagacccctgagcagacactggtgggcttcatgaatcagccagcaatgaagagccagctggccgccgccctgccaaggcacatgaccgccgatcggatgatcagaatcgtgaccacagagatccgcaagacaccacagctggcccagtgcgaccagagctccttcatcggcgccgtggtgcagtgttctcagctgggcctggagcctggcagcgccctgggccacgcctacctgctgccatttggcaacggccggtccaagtctggccagagcaatgtgcagctgatcatcggctatagaggcatgatcgatctggcccggagatccggacagatcgtgagcctgtccgccagggtggtgcgcgcagacgatgagttctcttttgagtacggcctggatgagaacctggtgcaccggccaggcgagaatgaggacgcacccatcacccacgtgtatgcagtggcaagactgaaggacggaggcacccagttcgaagtgatgacagtgaagcaggtggagaaggtgaaggcccagtccaaggcctctagcaatggcccctgggtgacccactgggaggagatggccaagaaaaccgtgatcaggcgcctgtttaagtacctgcccgtgagcatcgagatgcagaaggccgtggtgctggatgagaaggccgagtctgacgtggatcaggacaacgcctctgtgctgagcgccgagtattccgtgctggagtctggcgacgaggccacaaat

SEQ ID NO: 88 - DNA, 813 bp, Pantoea brenneri
  source 1..813: mol_type = genomic DNA
cagcctggcatctactatgacatcagcaacgaggattatcacaggggagcaggcatcagcaagtcccagctggacgacatcgccatctccccagccatctaccagtggagaaagcacgcccccgtggacgaggagaaaaccgccgccctggatctgggcacagccctgcactgcctgctgctggagcctgacgagttctctaagaggtttcagatcggcccagaggtgaaccggagaaccacagccggcaaggagaaggagaaggagttcatcgagcggtgcgaggcagagggaatcaccccaatcacacacgacgataataggaagctgaagctgatgagggattccgccctggcccacccaatcgcaaggtggatgctggaggcacagggaaacgcagaggcctctatctattggaatgacagagatgccggcgtgctgagcaggtgccgccccgacaagatcatcaccgagttcaactggtgcgtggacgtgaagtccacagccgacatcatgaagttccagaaggacttctactcttacagataccacgtgcaggacgccttctattccgatggctacgagtctcactttcacgagacacccacattcgcctttctggccgtgtctaccagcatcgactgcggcaggtatcctgtgcaggtgtttatcatggaccagcaggcaaaggatgcaggaagggccgagtacaagagaaacatccacaccttcgccgagtgtctgagcaggaatgagtggcctggcatcgccacactgtccctgcctttttgggccaaggagctgcgcaatgag

SEQ ID NO: 89 - DNA, 807 bp, Pantoea dispersa
  source 1..807: mol_type = genomic DNA
tccaaccagccacctctggccaccgcagatctgcagaaaacccagcagtctaaccaggtggccaagacccctgagcagacactggtgggcttcatgaatcagccagcaatgaagagccagctggccgccgccctgccaaggcacatgaccgccgatcggatgatcagaatcgtgaccacagagatccgcaagacacccgccctggcccagtgcgaccagagctccttcatcggagcagtggtgcagtgtagccagctgggcctggagcctggctccgccctgggccacgcctacctgctgccatttggcaacggccggtccaagtctggccagagcaatgtgcagctgatcatcggctatagaggcatgatcgatctggcccggagatccggacagatcgtgagcctgtccgccagggtggtgcgcgcagacgatgagttctcttttgagtacggcctggatgagaacctgatccaccggccaggcgacaatgagtccgcccccatcacccacgtgtatgcagtggcaagactgaaggacggaggcacccagttcgaagtgatgacagccaagcaggtggagaaggtgaaggcccagtccaaggcctctagcaacggaccctgggtgacccactgggaggagatggccaagaaaaccgtgatcaggcgcctgtttaagtacctgcccgtgagcatcgagatgcagaaggccgtggtgctggacgagaaggccgagagcgacgtggatcaggacaatgcctctgtgctgagcgccgagtattccgtgctggagtctggcacaggcgag

SEQ ID NO: 90 - DNA, 813 bp, Pantoea dispersa
  source 1..813: mol_type = genomic DNA
gagccaggcatctactatgacatcagcaacgaggcctaccactccggccccggcatcagcaagtcccagctggacgacatcgccaggagccctgccatcttccagtggcgcaaggacgccccagtggataccgagaaaaccaaggccctggacctgggcaccgatttccactgcgccgtgctggagccagagaggtttgcagacatgtatcgcgtgggccctgaagtgaatcggagaaccacagccggcaaggccgaggagaaggagttctttgagaagtgtgagaaggatggagccgtgcccatcacccacgacgatgcacggaaggtggagctgatgagaggctccgtgatggcccaccctatcgccaagcagatgatcgcagcacagggacacgcagaggcctctatctactggcacgacgagagcacaggcaacctgtgccggtgtagacccgacaagtttatccctgattggaattggatcgtggacgtgaaaaccacagccgatatgaagaagttcaggcgcgagttttacgatctgcggtatcacgtgcaggacgccttctacaccgatggctatgccgcccagtttggcgagcggcctaccttcgtgtttgtggtgacatccaccacaatcgactgcggcagataccccaccgaggtgttctttctggatgaggagacaaaggccgccggcaggtctgagtaccagagcaacctggtgacctattccgagtgtctgtctcgcaatgagtggccaggcatcgccacactgtctctgccccactgggccaaggagctgaggaacgtg

SEQ ID NO: 91 - DNA, 810 bp, unidentified
  source 1..810: mol_type = genomic DNA; note = Type-F symbiont of Plautia stali
tccaaccagccccctatcgcctctgccgatctgcagaaaacccagcagtctaagcaggtggccaacaagacccctgagcagacactggtgggcttcatgaatcagccagcaatgaagtcccagctggccgccgccctgccaaggcacatgacagccgatcggatgatcagaatcgtgaccacagagatccgcaagacccccgccctggccacatgcgaccagagctccttcatcggagcagtggtgcagtgtagccagctgggcctggagcctggctccgccctgggccacgcctacctgctgccatttggcaacggccggtccaagtctggccagtctaatgtgcagctgatcatcggctatagaggcatgatcgacctggcccggagaagcggacagatcgtgagcctgtccgccagggtggtgcgcgcagacgatgagttctcctttgagtacggcctggatgagaacctgatccaccggccaggcgataatgaggacgcccccatcacccacgtgtatgcagtggcaagactgaaggacggaggcacccagttcgaagtgatgacagccaagcaggtggagaaggtgaaggcccagagcaaggcctctagcaacggaccctgggtgacccactgggaggagatggccaagaaaaccgtgatcaggcgcctgtttaagtacctgcccgtgagcatcgagatgcagaaggccgtggtgctggatgagaaggccgagagcgacgtggatcaggacaatgcctctgtgctgagcgccgagtattccgtgctggagggcgacggcggcgag

SEQ ID NO: 92 - DNA, 813 bp, unidentified
  source 1..813: mol_type = genomic DNA; note = Type-F symbiont of Plautia stali
cagcctggcatctactatgacatcagcaacgaggattatcacggcggccctggcatcagcaagtcccagctggacgacatcgccatctccccagccatctaccagtggaggaagcacgcccccgtggacgaggagaaaaccgccgccctggatctgggcacagccctgcactgcctgctgctggagcctgacgagttctctaagagatttgagatcggcccagaggtgaaccggagaaccacagccggcaaggagaaggagaaggagttcatggagaggtgtgaggcagagggagtgacccctatcacacacgacgataatcggaagctgagactgatgagggatagcgcaatggcccacccaatcgccagatggatgctggaggcacagggaaacgcagaggcctctatctattggaatgacagggataccggcgtgctgagcaggtgccgccccgacaagatcatcaccgacttcaactggtgcgtggacgtgaagtccacagccgacatcatcaagttccagaaggacttttactcttatcgctaccacgtgcaggacgccttctattccgatggctacgagtctcactttgacgagacaccaacattcgcctttctggccgtgtctacaagcatcgattgcggccggtatcccgtgcaggtgttcatcatggaccagcaggcaaaggatgcaggaagggccgagtacaagcggaacatccacacctttgccgagtgtctgagccgcaatgagtggcctggcatcgccacactgtccctgccttactgggccaaggagctgcggaatgag

SEQ ID NO: 93 - DNA, 801 bp, Providencia stuartii
  source 1..801: mol_type = genomic DNA
agcaacccacctctggcccaggcagacctgcagaaaacccagggcacagaggtgaaggagaaaaccaaggatcagatgctggtggagctgatcaataagccttccatgaaggcacagctggccgccgccctgccaaggcacatgacacccgaccggatgatcagaatcgtgaccacagagatcagaaagacccccgccctggccacatgcgatatgcagagcttcgtgggagcagtggtgcagtgttcccagctgggcctggagcctggcaacgccctgggacacgcctacctgctgccttttggcaacggcaagtctaagagcggccagtctaatgtgcagctgatcatcggctatcggggcatgatcgacctggcccggagaagcggccagatcgtgtccatctctgccaggaccgtgcgccagggcgataacttccactttgagtacggcctgaacgagaatctgacccacgtgcctggcgagaatgaggactctccaatcacacacgtgtacgcagtggcaaggctgaaggatggaggcgtgcagttcgaagtgatgacctataaccagatcgagaaggtgcgcgccagctccaaggcaggacagaatggaccctgggtgagccactgggaggagatggccaagaaaaccgtgatcaggcgcctgttcaagtacctgcccgtgtctatcgagatgcagaaggccgtgatcctggacgagaaggccgaggccaacatcgatcaggagaatgccaccatctttgagggcgagtatgaggaagtgggcacagacggcaag

SEQ ID NO: 94 - DNA, 810 bp, Providencia stuartii
  source 1..810: mol_type = genomic DNA
gagggcatctactataacatcagcaatgaggactaccacaacggcctgggcatctccaagtctcagctggatctgatcaatgagatgcctgccgagtatatctggtccaaggaggcccccgtggacgaggagaagatcaagcctctggagatcggcaccgccctgcactgcctgctgctggagccagacgagtaccacaagagatataagatcggccccgatgtgaaccggagaacaaatgccggcaaggagaaggagaaggagttctttgatatgtgcgagaaggagggcatcacccccatcacacacgacgataaccggaagctgatgatcatgagagactctgccctggcccaccctatcgccaagtggtgtctggaggccgatggcgtgagcgagagctccatctactggaccgacaaggagacagatgtgctgtgcaggtgtcgcccagaccgcatcatcaccgcccacaactacatcgtggatgtgaagtctagcggcgacatcgagaagttcgattacgagtactacaactacagataccacgtgcaggacgccttttactccgatggctataaggaggtgaccggcatcacccctacattcctgtttctggtggtgtctaccaagatcgactgcggcaagtaccccgtgcggacctacgtgatgagcgaggaggcaaagtccgccggaaggaccgcctacaagcacaacctgctgacctatgccgagtgtctgaaaaccgatgagtgggccggcatcaggacactgtctctgcccagatgggcaaaggagctgcggaatgag

SEQ ID NO: 95 - DNA, 801 bp, Providencia sp.
  source 1..801: mol_type = genomic DNA
tctaacccccctctggcccagagcgacctgcagaaaacccagggcacagaggtgaaggtgaaaaccaaggatcagcagctgatccagttcatcaatcagccttctatgaaggcacagctggccgccgccctgccaaggcacatgacacccgaccggatgatcagaatcgtgaccacagagatcagaaagacccccgccctggccacatgcgatatgcagtccttcgtgggcgccgtggtgcagtgttctcagctgggcctggagcctggcaacgccctgggacacgcctacctgctgccttttggcaacggcaaggccaagtccggccagtctaatgtgcagctgatcatcggctatcggggcatgatcgacctggcccggagatccaaccagatcatctctatcagcgccaggaccgtgcgccagggcgataacttccactttgagtacggcctgaatgaggacctgacccacacacctagcgagaatgaggattccccaatcacccacgtgtacgcagtggcaaggctgaaggacggaggcgtgcagtttgaagtgatgacatataaccaggtggagaaggtgcgcgccagctccaaggcaggacagaatggaccctgggtgagccactgggaggagatggccaagaaaaccgtgatcaggcgcctgttcaagtacctgcccgtgtccatcgagatgcagaaggcagtggtgctggacgagaaggcagaggccaacgtggatcaggagaatgccaccatctttgagggcgagtatgaggaagtgggcacagatggcaat

SEQ ID NO: 96 - DNA, 813 bp, Providencia sp.
  source 1..813: mol_type = genomic DNA
aaggagggcatctactataacatcagcaatgaggactaccacaacggcctgggcatctccaagtctcagctggatctgatcaatgagatgcctgccgagtatatctggtccaaggaggcccccgtggacgaggagaagatcaagcctctggagatcggcaccgccctgcactgcctgctgctggagccagacgagtaccacaagagatataagatcggccccgatgtgaaccggagaacaaatgtgggcaaggagaaggagaaggagttctttgatatgtgcgagaaggagggcatcacccccatcacacacgacgataaccggaagctgatgatcatgagagactctgccctggcccaccctatcgccaagtggtgtctggaggccgatggcgtgagcgagagctccatctactggaccgacaaggagacagatgtgctgtgcaggtgtcgcccagaccgcatcatcaccgcccacaactacatcatcgatgtgaagtctagcggcgacatcgagaagttcgattacgagtactacaactacagataccacgtgcaggacgccttttactccgatggctataaggaggtgaccggcatcacccctacattcctgtttctggtggtgtctaccaagatcgactgcggcaagtaccccgtgcggacctacgtgatgagcgaggaggcaaagtccgccggaaggaccgcctacaagcacaacctgctgacctatgccgagtgtctgaaaaccgatgagtgggccggcatcaggacactgtctctgcccagatgggcaaaggagctgcggaatgag

SEQ ID NO: 97 - DNA, 711 bp, Shewanella putrefaciens
  source 1..711: mol_type = genomic DNA
cagaccgcacaggtgaagctgagcgtgccccaccagcaggtgtaccaggacaacttcaattatctgagctcccaggtggtgggccacctggtggatctgaacgaggagatcggctacctgaaccagatcgtgtttaattctctgagcaccgcctctcccctggacgtggcagcaccttggagcgtgtacggcctgctgctgaacgtgtgccggctgggcctgtccctgaatccagagaagaagctggcctatgtgatgccctcctggtctgagacaggcgagatcatcatgaagctgtaccccggctataggggcgagatcgccatcgcctctaacttcaatgtgatcaagaacgccaatgccgtgctggtgtatgagaacgatcacttccgcatccaggcagcaaccggcgagatcgagcactttgtgacaagcctgtccatcgaccctagggtgcgcggagcatgcagcggaggctactgtcggtccgtgctgatggataatacaatccagatctcttatctgagcatcgaggagatgaacgccatcgcccagaatcagatcgaggccaacatgggcaataccccttggaactccatctggcggacagagatgaatagagtggccctgtaccggagagcagcaaaggactggaggcagctgatcaaggccaccccagagatccagtccgccctgtctgatacagagtat

SEQ ID NO: 98 - DNA, 1173 bp, Shewanella putrefaciens
  source 1..1173: mol_type = genomic DNA
ggcaccgccctggcccagacaatcagcctggactggcaggataccatccagccagcatacacagcctccggcaagcctaacttcctgaatgcccagggcgagatcgtggagggcatctacaccgatctgcctaattccgtgtatcacgccctggacgcacacagctccaccggcatcaagacattcgccaagggccgccaccactactttcggcagtatctgtctgacgtgtgccggcagagaacaaagcagcaggagtacaccttcgacgccggcacctacggccacatgctggtgctggagccagagaacttccacggcaacttcatgaggaaccccgtgcctgacgattttccagacatcgagctgatcgagagcatcccacagctgaaggccgccctggccaagagcaacctgcccgtgtccggagcaaaggccgccctgatcgagagactgtacgccttcgacccatccctgcccctgtttgagaagatgagggagaaggccatcaccgactatctggatctgcgctacgccaagtatctgcggaccgacgtggagctggatgagatggccacattctacggcatcgatacctctcagacacgggagaagaagatcgaggagatcctggccatctctcctagccagccaatctgggagaagctgatcagccagcacgtgatcgaccacatcgtgtgggacgatgccatgagggtggagagatccaccagggcccaccctaaggcagactggctgatctctgatggctatgccgagctgacaatcatcgcaaggtgcccaaccaccggcctgctgctgaaggtgcggtttgactggctgaggaatgatgccatcggcgtggacttcaagaccacactgtctaccaaccccacaaagtttggctaccagatcaaggacctgcggtatgatctgcagcaggtgttctactgttatgtggccaatctggccggcatccctgtgaagcacttctgctttgtggccaccgagtacaaggacgccgataactgtgagacatttgagctgtctcacaagaaagtgatcgagagcaccgaggagatgttcgacctgctggatgagtttaaggaggccctgacctccggcaattggtatggccacgacaggtcccgctctacatgggtcatcgaggtg

SEQ ID NO: 99 - DNA, 888 bp, Bacillus sp.
  source 1..888: mol_type = genomic DNA
agcaagcagctgaccacagtgaatacccaggccgtggtgggcacattctcccaggccgagctggataccctgaagcagacaatcgccaagggcaccacaaacgagcagttcgccctgtttgtgcagacctgcgccaactctaggctgaatccatttctgaaccacatccactgtatcgtgtataacggcaaggagggcgccaccatgagcctgcagatcgcagtggagggcatcctgtacctggcacgcaagacagacggctataagggcatcgagtgccagctgatccacgagaatgacgagttcaagtttgatgccaagtccaaggaggtggatcaccagatcggattccccaggggcaacgtgatcggaggatatgcaatcgcaaagagggagggctttgacgatgtggtggtgctgatggagtctaacgaggtggaccacatgctgaagggccggaatggccacatgtggagagactggttcaacgatatgtttaagaagcacatcatgaagcgggccgccaagctgcagtacggcatcgagatcgcagaggacgagacagtgagcagcggacctagcgtggataatatcccagagtataagccacagccccggaaggacatcacacccaaccaggacgtgatcgatgccccccctcagcagcctaagcaggacgatgaggccgccaagctgaaggccgccagatctgaggtgagcaagaagttcaagaagctgggcatcgtgaaggaggatcagaccgagtacgtggagaagcacgtgcctggcttcaagggcacactgtccgactttatcggcctgtctcagctgctggatctgaatatcgaggcccaggaggcccagtccgccgacggcgatctgctggac

SEQ ID NO: 100 - DNA, 1323 bp, Bacillus sp.
  source 1..1323: mol_type = genomic DNA
acctacgccgccgacgagacactggtgcagctgctgctgtccgtggatggcaagcagctgctgctgggaaggggcctgaagaagggcaaggcccagtactatatcaatgaggtgccatctaaggccaaggagttcgaggagatccgggaccagctgtttgacaaggatctgttcatgtccctgtttaacccctcttacttctttaccctgcactgggagaagcagagggccatgatgctgaagtatgtgacagcccccgtgtctaaggaggtgctgaagaatctgcctgaggcccagtccgaggtgctggagagatacctgaagaagcactctctggtggatctggagaagatccacaaggacaacaagaataagcaggataaggcctatatctctgcccagagcaggaccaacacactgaaggagcagctgatgcagctgaccgaggagaagctggacatcgattccatcaaggccgagctggcccacatcgacatgcaggtcatcgagctggagaagcagatggatacagccttcgagaagaaccaggcctttaatctgcaggcccagatcaggaatctgcaggacaagatcgagatgagcaaggagcggtggccctccctgaagaacgaagtgatcgaggatacctgccggacatgcaagcggcccctggacgaggatagcgtggaggccgtgaaggccgacaaggataatcggatcgccgagtacaaggccaagcacaactccctggtgtctcagagaaatgagctgaaggagcagctgaacaccatcgagtatatcgacgtgacagagctgagagagcagatcaaggagctggatgagtccggacagcctctgagggagcaggtgcgcatctacagccagtatcagaatctggacacccaggtgaagtccgccgaggcagacgagaacggcatcctgcaggatctgaaggcctctatcttcatcctggatagcatcaaggcctttaggggcaaggaggccgagatgcaggccgagaaggtgcaggccctgttcaccacactgagcgtgcgcctgtttaagcagaataagggcgacggcgagatcaagccagatttcgagatcgagatgaacgacaagccctatcggaccctgagcctgtccgagggcatccgggcaggcctggagctgcgggacgtgctgagccagcagtccgagctggtgacccctacattcgtggataatgccgagtctatcaccagcttcaagcagccaaacggccagctgatcatcagccgggtggtggcaggacaggagctgaagatcgaggccgtgagcgag

SEQ ID NO: 101 - DNA, 804 bp, Shigella sonnei
  source 1..804: mol_type = genomic DNA
accaagcagccccctatcgccaaggccgacctgcagaaaacccaggagaacagggcaccagcagccatcaagaacaatgatgtgatctcctttatcaatcagccctctatgaaggagcagctggccgccgccctgcctaggcacatgaccgccgagaggatgatccgcatcgccaccacagagatccgcaaggtgcctgccctgggcaactgcgacacaatgagcttcgtgagcgccatcgtgcagtgtagccagctgggcctggagccaggctccgccctgggccacgcctacctgctgcccttcggcaacaagaatgagaagtccggcaagaagaatgtgcagctgatcatcggctataggggcatgatcgatctggcccggagatctggccagatcgcctctctgagcgccagagtggtgcgggagggcgacgagttcaactttgagttcggcctggatgagaagctgatccaccggcctggcgagaatgaggacgccccagtgacccacgtgtacgcagtggccagactgaaggatggcggcacccagtttgaagtgatgacaaggcgccagatcgagctggtgaggtcccagtctaaggccggcaacaatggcccttgggtgacccactgggaggagatggccaagaaaaccgccatccggagactgttcaagtacctgccagtgtctatcgagatccagcgcgccgtgagcatggacgagaaggagccactgaccatcgaccccgccgatagctccgtgctgacaggcgagtattctgtgatcgataacagcgaggag

SEQ ID NO: 102 - DNA, 1944 bp, Shigella sonnei
  source 1..1944: mol_type = genomic DNA
gatcgcggcctgctgacaaaggagtggaggaagggaaaccgggtgagccggatcaccaggacagccagcggagcaaacgcaggaggaggaaatctgaccgacagaggcgagggcttcgtgcacgatctgacaagcctggcccgcgacatcgcaaccggcgtgctggcccggagcatggacgtggacatctacaacctgcaccctgcccacgccaagaggatcgaggagatcatcgccgagaataagccccctttcagcgtgtttagagacaagtttatcacaatgccaggcggcctggactactccagggccatcgtggtggcctctgtgaaggaggccccaatcggcatcgaagtgatccccgcccacgtgaccgcctatctgaacaaggtgctgaccgagacagaccacgccaatccagatcccgagatcgtggacatcgcatgcggcagaagctccgcccctatgccacagagggtgaccgaggagggcaagcaggacgatgaggagaagctgcagccttctggcaccacagcagatgagcagggagaggcagagacaatggagccagacgccacaaagcaccaccaggatacccagcctctggacgcccagagccaggtgaacagcgtggatgccaagtatcaggagctgagagccgagctgcacgaggccaggaagaacatcccttccaagaatccagtggacgcagataagctgctggccgcctctcgcggcgagttcgtggacggcatcagcgacccaaacgatcccaagtgggtgaagggcatccagacacgggattccgtgtaccagaatcagcctgagacagagaaaaccagccccgacatgaagcagccagagcctgtggtgcagcaggagcctgagatcgccttcaacgcctgcggacagaccggcggcgacaattgcccagattgtggcgccgtgatgggcgatgccacctatcaggagacatttgacgaggagaaccaggtggaggccaaggagaatgatcctgaggagatggagggcgccgagcacccacacaacgagaatgccggcagcgacccccacagagactgttccgatgagacaggcgaggtggccgatcccgtgatcgtggaggacatcgagcctggcatctactatggcatcagcaacgagaattaccacgcaggccccggcgtgtccaagtctcagctggacgacatcgccgacacacctgccctgtatctgtggaggaagaacgccccagtggataccacaaagaccaagacactggacctgggcaccgcattccactgccgcgtgctggagccagaggagttcagcaatcggtttatcgtggcccccgagttcaaccggagaacaaatgccggcaaggaggaggagaaggcctttctgatggagtgtgcctccacaggcaagatggtcatcaccgccgaggagggcagaaagatcgagctgatgtaccagtctgtgatggcactgccactgggacagtggctggtggagagcgccggacacgcagagtctagcatctattgggaggaccccgagacaggcatcctgtgcaggtgtcgccccgacaagatcatccctgagttccactggatcatggacgtgaaaaccacagccgacatccagcggttcaagacagcctactatgattacaggtatcacgtgcaggatgccttctactccgacggctatgaggcccagtttggcgtgcagcccaccttcgtgtttctggtggcctctaccacaatcgagtgcggcagataccccgtggagatctttatgatgggagaggaggcaaagctggccggacagctggagtatcaccgcaacctgcggacactggccgattgtctgaataccgacgagtggccagccatcaagaccctgtccctgcccagatgggcaaaggagtacgccaacgac

SEQ ID NO: 103 - DNA, 828 bp, Salmonella enterica
  source 1..828: mol_type = genomic DNA
accaagcagccccctatcgccaaggccgacctgcagaaaacccagggaaacagggcacctgcagcagtgaatgacaaggatgtgctgtgcgtgatcaacagccctgccatgaaggcacagctggccgccgccctgccaaggcacatgaccgccgagaggatgatccgcatcgccaccacagagatcaggaaggtgccagagctgcgcaactgcgacagcaccagcttcatcggcgccatcgtgcagtgttctcagctgggcctggagcccggcagcgccctgggccacgcctacctgctgccttttggcaatggcaaggccaagaacggcaagaagaatgtgcagctgatcatcggctatcggggcatgatcgatctggcccggagatctggccagatcatctccctgagcgccagagtggtgcgggagtgtgacgagttctcctacgagctgggcctggatgagaagctggtgcaccggccaggcgagaacgaggacgcacccatcacccacgtgtatgccgtggccaagctgaaggatggcggcgtgcagtttgaagtgatgaccaagaagcaggtggagaaggtgagagatacacactccaaggccgccaagaatgccgcctctaagggcgccagctccatctgggacgagcacttcgaggatatggccaagaaaaccgtgatccggaagctgtttaagtacctgcccgtgagcatcgagatccagagagccgtgagcatggacggcaaggaggtggagacaatcaacccagacgacatcagcgtgatcgccggcgagtattccgtgatcgataatcccgaggag

SEQ ID NO: 104 - DNA, 1944 bp, Salmonella enterica
  source 1..1944: mol_type = genomic DNA
gatcgcggcctgctgacaaaggagtggaggaagggaaaccgggtgagccggatcaccaggacagccagcggagcaaacgcaggaggaggaaatctgaccgacagaggcgagggcttcgtgcacgatctgacaagcctggcccgcgacgtggcaaccggcgtgctggcccggagcatggacgtggacatctacaacctgcaccctgcccacgccaagagggtggaggagatcatcgccgagaataagccccctttcagcgtgtttagagacaagtttatcacaatgcctggcggcctggactactccagggccatcgtggtggcctctgtgaaggaggcccctatcggcatcgaagtgatcccagcccacgtgaccgagtatctgaacaaggtgctgaccgagacagaccacgccaatccagatcccgagatcgtggacatcgcatgcggcagaagctccgcccctatgccacagagggtgaccgaggagggcaagcaggacgatgaggagaagccccagccttctggagctatggccgacgagcaggcaaccgcagagacagtggagccaaacgccacagagcaccaccagaatacccagcccctggatgcccagagccaggtgaactccgtggacgccaagtatcaggagctgagagccgagctgcaggaggccaggaagaacatcccctccaagaatcctgtggacgcagataagctgctggccgcctctcgcggcgagttcgtggatggcatcagcgaccctaacgatccaaagtgggtgaagggcatccagacacgggattccgtgtaccagaatcagcccgagacagagaagatctctcctgacgccaagcagccagagcccgtggtgcagcaggagcccgagacagtgtgcaacgcctgtggacagaccggcggcgacaattgccctgattgtggcgccgtgatgggcgacgccacatatcaggagacattcggcgaggagaatcaggtggaggccaaggagaaggaccccgaggagatggagggagcagagcaccctcacaacgagaatgccggcagcgacccacacagagactgttccgatgagacaggcgaggtggccgatccagtgatcgtggaggacatcgagcctggcatctactatggcatcagcaacgagaattaccacgcaggccccggcgtgtccaagtctcagctggacgacatcgccgacacacccgccctgtatctgtggaggaagaacgcccctgtggataccacaaagaccaagacactggacctgggcaccgcattccactgccgcgtgctggagcctgaggagttcagcaatcggtttatcgtggccccagagttcaaccggagaacaaatgccggcaaggaggaggagaaggcctttctgatggagtgtgcctccaccggcaagacagtgatcaccgccgaggagggcagaaagatcgagctgatgtaccagtctgtgatggcactgcctctgggacagtggctggtggagagcgccggacacgcagagtctagcatctattgggaggaccccgagacaggcatcctgtgcaggtgtcgcccagacaagatcatccccgagttccactggatcatggacgtgaaaaccacagccgacatccagcggttcaagacagcctactatgattacaggtatcacgtgcaggatgccttctactccgacggctatgaggcccagtttggcgtgcagccaaccttcgtgtttctggtggcctctaccacagtggagtgcggcagataccccgtggagatctttatgatgggagaggaggcaaagctggccggacagcaggagtatcaccgcaacctgcggacactggccgattgtctgaataccgacgagtggcctgccatcaagaccctgtccctgccacggtgggccaaggagtacgccaacgac

SEQ ID NO: 105 - DNA, 993 bp, Acetobacter sp.
  source 1..993: mol_type = genomic DNA
aacgccccccagaagcagaataccagagccgccgtgaagaagatcagccctcaggagttcgccgagcagtttgccgccatcatcccacaggtgaagtccgtgctgcccgcccacgtgaccttcgagaagtttgagcgggtggtgagactggccgtgcggaagaaccctgacctgctgacatgctccccagcctctctgttcatggcatgtatccaggcagcctccgacggcctgctgcctgatggaagggagggagcaatcgtgagccggtggagctccaagaagagctgcaacgaggcctcctggatgccaatggtggccggcctgatgaagctggcccggaacagcggcgacatcgccagcatctctagccaggtggtgttcgagggcgagcactttagagtggtgctgggcgacgaggagaggatcgagcacgagcgcgatctgggcaagaccggcggcaagatcgtggcagcctacgccgtggcaaggctgaaggacggcagcgatccaatccgcgagatcatgtcctggggccagatcgagaagatcagaaacacaaataagaagtgggagtggggaccctggaaggcctgggaggacgagatggccagaaagaccgtgatccggagactggccaagagactgcccatgtctacagataaggagggagagaggctgcgcagcgccatcgagaggatcgactccctggtggacatctctgccaacgtggacgcacctcagatcgcagcagacgatgagtttgccgccgccgcccacggcgtggagccacagcagatcgcagcacctgacctgatcggccgcctggcccagatgcagtccctggagcaggtgcaggacatcgagccccaggtgtctcacgccatccaggaggccgacaagaggggcgacagcgatacagccaatgccctggatgccgccctgcagagcgccctgtcccgcacctctacagccaaggaggaggtgcctgcc

SEQ ID NO: 106 - DNA, 897 bp, Acetobacter sp.
  source 1..897: mol_type = genomic DNA
gtgatctctaagagcggcatctacgacctgaccaacgagcagtatcacgccgatccttgcccagagatgtccctgagctcctctggagccagggacctgctgagctcctgtcctgccaagttcatcgccgccaagcagctgccacagcagaataagaggtgctttgacatcggctctgccggacacctgatggtgctggagccacacctgttcgaccagaaggtgtgcgagatcaagcaccctgattggcgcacaaaggcagcaaaggaggagcgggacgccgcctacgccgagggaagaatccccctgctgagccgcgaggtggaggacatcagggcaatgcactccgtggtgtggagagattctctgggagccagggccttcagcggaggcaaggcagagcagtccctggtgtggcgcgacgaggagtttggcatctggtgccggctgcggcccgattacgtgcctaacaatgccgtgcggatcttcgactataagaccgccacaaacggctcccccgatgcctttatgaaggagatctacaatcggggctatcaccagcaggccgcctggtatctggacggatatgaggcagtgaccggccacaggccacgcgagttctggtttgtggtgcaggagaaaaccgcccccttcctgctgtctttctttcagatggatgagatgagcctggagatcggccggaccctgaacagacaggccaagggcatctttgcctggtgcctgcgcaacaattgttggccaggctatcagcccgaggtggatggcaaggtgagattctttaccacatctccccctgcctggctggtgagggagtacgagtttaagaatgagcacggcgcctatgagccacccgagatcaagcggaaggaggtggcc

SEQ ID NO: 107 - DNA, 825 bp, Salmonella enterica
  source 1..825: mol_type = genomic DNA
ccaaagcagccccctatcgccaaggcagacctgcagaaaacccagggagcacggaccccaacagcagtgaagaacaataacgatgtgatctcctttatcaatcagccttctatgaaggagcagctggccgccgccctgccaaggcacatgaccgccgagcggatgatcagaatcgccaccacagagatcaggaaggtgcccgccctgggcgactgcgatacaatgtcttttgtgagcgccatcgtgcagtgtagccagctgggcctggagcctggcggcgccctgggccacgcctacctgctgcctttcggcaatcggaacgagaagtccggcaagaagaatgtgcagctgatcatcggctatagaggcatgatcgacctggcccggagatccggacagatcgccagcctgtccgccagggtggtgcgcgagggcgacgatttctcttttgagttcggcctggaggagaagctggtgcacaggccaggcgagaacgaggacgcccccgtgacccacgtgtacgcagtggcacgcctgaaggatggaggcacccagtttgaagtgatgacacggaagcagatcgagctggtgagagcccagtctaaggccggcaataacggcccttgggtgacccactgggaggagatggccaagaaaaccgccatcaggcgcctgttcaagtacctgcccgtgagcatcgagatccagagggccgtgagcatggatgagaaggagacactgacaatcgacccagccgatgccagcgtgatcaccggcgagtattccgtggtggagaatgccggcgtggaggagaacgtgacagcc

SEQ ID NO: 108 - DNA, 801 bp, Salmonella enterica
  source 1..801: mol_type = genomic DNA
tactatgacatcccaaacgaggcctaccacgcaggccccggcgtgtctaagagccagctggacgacatcgccgatacccccgccatctatctgtggcggaagaatgcccctgtggacaccgagaaaaccaagtccctggataccggcacagccttccactgcagggtgctggagccagaggagttcagcaagcggttcatcatcgcccccgagttcaaccggagaacctccgccggcaaggaggaggagaaaaccttcctggaggagtgtacccggacaggcagaaccgtgctgacagccgaggagggcaggaagatcgagctgatgtaccagtccgtgatggcactgccactgggacagtggctggtggagtctgccggctacgccgagagctccgtgtattgggaggaccctgagacaggcatcctgtgccggtgtagacccgataagatcatccctgagttccactggatcatggacgtgaaaaccacagccgacatccagaggtttcgcaccgcctactatgactacagataccacgtgcaggacgccttctactctgatggctatagagcccagtttggcgagatccctacattcgtgtttctggtggccagcaccacagcagagtgcggcagataccccgtggagatctttatgatgggagaggacgcaaagctggccggacagcgcgagtataggcgcaatctgcagaccctggccgagtgtctgaacaatgatgagtggcctgccatcaagacactgtctctgccacggtgggccaaggagaacgccaatgcc

SEQ ID NO: 109 - DNA, 723 bp, Pseudobacteriovorax antillogorgiicola
  source 1..723: mol_type = genomic DNA
ggccacctggtgagcaagaccgagcaggattacatcaagcagcactatgccaagggcgccacagaccaggagttcgagcactttatcggcgtgtgcagggccagaggcctgaacccagccgccaatcagatctacttcgtgaagtatcggtccaaggatggaccagcaaagccagcctttatcctgtctatcgacagcctgaggctgatcgcacaccgcaccggcgattacgcaggatgctctgagcccatcttcacagacggcggcaaggcctgtaccgtgacagtgcggagaaacctgaagagcggcgagacaggcaatttctccggcatggccttttatgacgagcaggtgcagcagaagaacggccggcctacctccttttggcagtctaagccaagaacaatgctggagaagtgtgcagaggcaaaggccctgaggaaggccttccctcaggatctgggccagttttacatcagagaggagatgccccctcagtatgacgagcctatccaggtgcacaagccaaaggccctggaggagcccaggttcagcaagtccgatctgtccaggcgcaagggcctgaacaggaagctgtctgccctgggagtggaccccagccgcttcgatgaggtggccacctttctggacggcacacctgatcgcgagctgggccagaagctgaagctgtggctgaaggaggccggctacggcgtgaatcag

SEQ ID NO: 110 - DNA, 897 bp, Pseudobacteriovorax antillogorgiicola
  source 1..897: mol_type = genomic DNA
agcaagctgtccaacctgaaggtgtctaatagcgacgtggatacactgagccggatcagaatgaaggagggcgtgtatcgggacctgccaatcgagagctaccaccagtcccccggctattctaagaccagcctgtgccagatcgataaggcccctatctacctgaaaaccaaggtgccacagaagtccacaaagtctctgaacatcggcaccgccttccacgaggctatggagggcgtgtttaaggacaagtatgtggtgcaccccgatcctggcgtgaataagaccacaaagtcttggaaggacttcgtgaagaggtatcctaagcacatgccactgaagcgcagcgagtacgaccaggtgctggccatgtacgatgccgcccggtcttatagaccttttcagaagtaccacctgagccggggcttctacgagagctccttttattggcacgatgccgtgacaaacagcctgatcaagtgcagacccgactatatcacccctgatggcatgagcgtgatcgacttcaagaccacagtggaccccagccccaagggctttcagtaccaggcctacaagtatcactactacgtgagcgccgccctgaccctggagggaatcgaggcagtgaccggcatcaggccaaaggagtacctgttcctggccgtgtccaattctgccccatacctgaccgccctgtatcgcgcctctgagaaggagatcgccctgggcgaccactttatccggcggagcctgctgaccctgaaaacctgtctggagtctggcaagtggcccggcctgcaggaggagatcctggagctgggcctgcctttctccggcctgaaggagctgagagaggagcaggaggtggaggatgagtttatggagctggtgggc

SEQ ID NO: 111 - DNA, 669 bp, Photobacterium sp.
  source 1..669: mol_type = genomic DNA
aacaccgacatgatcgccatgcccccttctccagccatcagcatgctggacacaagcaagctggatgtgatggtgcgggcagcagagctgatgtcccaggccgtggtcatggtgcccgaccacttcaagggcaagccagccgattgcctggcagtggtcatgcaggcagaccagtggggcatgaacccctttaccgtggcccagaaaacccacctggtgagcggcaccctgggatacgagtcccagctggtgaatgccgtgatcagctcctctaaggccatcaagggccggttccactatgagtggtctgatggctgggagagactggccggcaaggtgcagtacgtgaaggagtctcggcagagaaagggccagcagggcagctatcaggtgaccgtggccaagccaacatggaagccagaggacgagcagggcctgtgggtgcggtgtggagccgtgctggccggagagaaggacatcacatggggccctaagctgtacctggccagcgtgctggtgcggaacagcgagctgtggaccacaaagccctaccagcaggccgcctataccgccctgaaggattggtcccgcctgtatacacctgccgtgatgcagggctctatgaccggcaagagctggtccctgacaggcaggctgatcagcccccgc

SEQ ID NO: 112 - DNA, 663 bp, Photobacterium sp.
  source 1..663: mol_type = genomic DNA
gccgagcgggtgagaacctatcagcgggacgccgtgttcgcacacgagctgaaggccgagtttgatgaggccgtggagaacggcaagaccggcgtgacactggaggaccaggccagggccaagaggatggtgcacgaggccaccacaaaccccgcctctcggaattggttcagatacgacggagagctggccgcatgcgagaggagctatttttggcgcgatgaggaggcaggcctggtgctgaaggccaggcctgacaaggagatcggcaacaatctgatcgatgtgaagtccatcgaggtgccaaccgacgtgtgcgcctgtgatctgaacgcctatatcaatcggcagatcgagaagagaggctaccacatctccgccgcccactatctgtctggcacaggcaaggaccgcttcttttggatcttcatcaataaggtgaagggctacgagtgggtggcaatcgtggaggcctctcccctgcacatcgagctgggcacctatgaggtgctggagggcctgcggagcatcgccagctccacaaaggaggcagattacccagcacctctgtcccaccctgtgaacgagagaggcatcccacagcccctgatgtctaatctgagcacatacgccatgaagaggctggagcagtttcgcgagctg

SEQ ID NO: 113 - DNA, 693 bp, Providencia alcalifaciens
  source 1..693: mol_type = genomic DNA
aaggcacagctggccgccgccctgcctaagcacatcaccagcgaccggatgatcagaatcgtgtccaccgagatcagaaagaccccatctctggccaactgcgacatccagagcttcatcggcgccgtggtgcagtgttctcagctgggcctggagccaggcaacgccctgggacacgcctacctgctgccctttggcaatggcaagtccgacaacggcaagtctaatgtgcagctgatcatcggctatcggggcatgatcgatctggcccggagaagcggccagatcatctctatcagcgccaggaccgtgcgccagggcgacaacttccactttgagtacggcctgaacgagaatctgacccacatccccgagggcaatgaggactcccctatcacacacgtgtacgcagtggcacggctgaaggatgagggcgtgcagttcgaagtgatgacatataaccagatcgagaaggtgagagatagctccaaggccggcaagaatggcccctgggtgacccactgggaggagatggccaagaaaaccgtgatcaggcgcctgtttaagtacctgcccgtgagcatcgagatgcagaaggccgtgatcctggacgagaaggccgaggccaatatcgagcaggatcactccgccatcttcgaggccgagtttgaggaggtggactctaacggcaat

SEQ ID NO: 114 - DNA, 828 bp, Providencia alcalifaciens
  source 1..828: mol_type = genomic DNA
aacgagggcatctactatgacatctctaatgaggactatcaccacggcctgggcatctctaagagccagctggatctgatcgacgagagccccgccgatttcatctggcaccgggatgcccctgtggacaacgagaaaaccaaggccctggattttggcacagccctgcactgcctgctgctggagccagacgagttccagaagaggtttcgcatcgcccccgaggtgaaccggagaacaaatgccggcaaggagcaggagaaggagttcctggagatgtgcgagaaggagaatatcacccccatcacaaacgaggataataggaagctgtctctgatgaaggacagcgcaatggcccaccctatcgcccgctggtgtctggaggccaagggcatcgccgagagctccatctattggaaggacaaggatacagacatcctgtgccggtgtagaccagacaagctgatcgaggagcaccactggctggtggatgtgaagtccaccgccgacatccagaagttcgagcggtctatgtacgagtatagataccacgtgcaggattccttttattctgacggctacaagagcctgacaggcgagatgcccgtgttcgtgttcctggccgtgtccaccgtgatcaactgcggcagataccccgtgcgggtgttcgtgctggacgagcaggcaaagtccgtgggacggatcacctataagcagaatctgtttacatacgccgagtgtctgaaaaccgacgagtgggccggcatcagaaccctgagcctgccctcctgggcaaaggagctgaagcacgagcacaccacagcctct

SEQ ID NO: 115 - protein, 271 aa, Pantoea stewartii
MSNQPPIASADLQKANTGKQVANKTPEQTLVGFMNQPAMKSQLAAALPRHMTADRMIRIVTTEIRKTPALATCDQSSFIGAVVQCSQLGLEPGSALGHAYLLPFGNGRSKSGQSNVQLIIGYRGMIDLARRSGQIVSLSARVVRADDEFSFEYGLDENLIHRPGENEDAPITHVYAVARLKDGGTQFEVMTVKQIEKVKAQSKASSNGPWVTHWEEMAKKTVIRRLFKYLPVSIEMQKAVILDEKAESDVDQDNASVLSAEYSVLDGSSEE

SEQ ID NO: 116 - protein, 272 aa, Pantoea stewartii
MQPGVYYDISNEEYHAGPGISKSQLDDIAVSPAIFQWRKSAPVDDEKTAALDLGTALHCLLLEPDEFSKRFMIGPEVNRRTNAGKQKEQDFLDMCEQQGITPITHDDNRKLRLMRDSAFAHPVARWMLETEGKAEASIYWNDRDTQILSRCRPDKLITEFSWCVDVKSTADIGKFQKDFYSYRYHVQDAFYSDGYEAQFCEVPTFAFLVVSSSIDCGRYPVQVFIMDQQAKDAGRAEYKRNLTTYAECQARNEWPGIATLSLPYWAKEIRNV

SEQ ID NO: 117 - protein, 273 aa, Pantoea brenneri; identical to SEQ ID NO: 10

SEQ ID NO: 118 - protein, 272 aa, Pantoea brenneri; identical to SEQ ID NO: 4

SEQ ID NO: 119 - protein, 270 aa, Pantoea dispersa
MSNQPPLATADLQKTQQSNQVAKTPEQTLVGFMNQPAMKSQLAAALPRHMTADRMIRIVTTEIRKTPALAQCDQSSFIGAVVQCSQLGLEPGSALGHAYLLPFGNGRSKSGQSNVQLIIGYRGMIDLARRSGQIVSLSARVVRADDEFSFEYGLDENLIHRPGDNESAPITHVYAVARLKDGGTQFEVMTAKQVEKVKAQSKASSNGPWVTHWEEMAKKTVIRRLFKYLPVSIEMQKAVVLDEKAESDVDQDNASVLSAEYSVLESGTGE

SEQ ID NO: 120 - protein, 272 aa, Pantoea dispersa
MEPGIYYDISNEAYHSGPGISKSQLDDIARSPAIFQWRKDAPVDTEKTKALDLGTDFHCAVLEPERFADMYRVGPEVNRRTTAGKAEEKEFFEKCEKDGAVPITHDDARKVELMRGSVMAHPIAKQMIAAQGHAEASIYWHDESTGNLCRCRPDKFIPDWNWIVDVKTTADMKKFRREFYDLRYHVQDAFYTDGYAAQFGERPTFVFVVTSTTIDCGRYPTEVFFLDEETKAAGRSEYQSNLVTYSECLSRNEWPGIATLSLPHWAKELRNV

SEQ ID NO: 121 - protein, 271 aa, unidentified; identical to SEQ ID NO: 11
  source 1..271: note = Type-F symbiont of Plautia stali

SEQ ID NO: 122 - protein, 272 aa, unidentified; identical to SEQ ID NO: 5
  source 1..272: note = Type-F symbiont of Plautia stali

SEQ ID NO: 123 - protein, 268 aa, Providencia stuartii
MSNPPLAQADLQKTQGTEVKEKTKDQMLVELINKPSMKAQLAAALPRHMTPDRMIRIVTTEIRKTPALATCDMQSFVGAVVQCSQLGLEPGNALGHAYLLPFGNGKSKSGQSNVQLIIGYRGMIDLARRSGQIVSISARTVRQGDNFHFEYGLNENLTHVPGENEDSPITHVYAVARLKDGGVQFEVMTYNQIEKVRASSKAGQNGPWVSHWEEMAKKTVIRRLFKYLPVSIEMQKAVILDEKAEANIDQENATIFEGEYEEVGTDGK

SEQ ID NO: 124 - protein, 270 aa, Providencia stuartii
EGIYYNISNEDYHNGLGISKSQLDLINEMPAEYIWSKEAPVDEEKIKPLEIGTALHCLLLEPDEYHKRYKIGPDVNRRTNAGKEKEKEFFDMCEKEGITPITHDDNRKLMIMRDSALAHPIAKWCLEADGVSESSIYWTDKETDVLCRCRPDRIITAHNYIVDVKSSGDIEKFDYEYYNYRYHVQDAFYSDGYKEVTGITPTFLFLVVSTKIDCGKYPVRTYVMSEEAKSAGRTAYKHNLLTYAECLKTDEWAGIRTLSLPRWAKELRNE

SEQ ID NO: 125 - protein, 268 aa, Providencia sp.; identical to SEQ ID NO: 12

SEQ ID NO: 126 - protein, 272 aa, Providencia sp.; identical to SEQ ID NO: 6

SEQ ID NO: 127 - protein, 238 aa, Shewanella putrefaciens
MQTAQVKLSVPHQQVYQDNFNYLSSQVVGHLVDLNEEIGYLNQIVFNSLSTASPLDVAAPWSVYGLLLNVCRLGLSLNPEKKLAYVMPSWSETGEIIMKLYPGYRGEIAIASNFNVIKNANAVLVYENDHFRIQAATGEIEHFVTSLSIDPRVRGACSGGYCRSVLMDNTIQISYLSIEEMNAIAQNQIEANMGNTPWNSIWRTEMNRVALYRRAAKDWRQLIKATPEIQSALSDTEY

SEQ ID NO: 128 - protein, 392 aa, Shewanella putrefaciens
MGTALAQTISLDWQDTIQPAYTASGKPNFLNAQGEIVEGIYTDLPNSVYHALDAHSSTGIKTFAKGRHHYFRQYLSDVCRQRTKQQEYTFDAGTYGHMLVLEPENFHGNFMRNPVPDDFPDIELIESIPQLKAALAKSNLPVSGAKAALIERLYAFDPSLPLFEKMREKAITDYLDLRYAKYLRTDVELDEMATFYGIDTSQTREKKIEEILAISPSQPIWEKLISQHVIDHIVWDDAMRVERSTRAHPKADWLISDGYAELTIIARCPTTGLLLKVRFDWLRNDAIGVDFKTTLSTNPTKFGYQIKDLRYDLQQVFYCYVANLAGIPVKHFCFVATEYKDADNCETFELSHKKVIESTEEMFDLLDEFKEALTSGNWYGHDRSRSTWVIEV

SEQ ID NO: 129 - protein, 297 aa, Bacillus sp.
MSKQLTTVNTQAVVGTFSQAELDTLKQTIAKGTTNEQFALFVQTCANSRLNPFLNHIHCIVYNGKEGATMSLQIAVEGILYLARKTDGYKGIECQLIHENDEFKFDAKSKEVDHQIGFPRGNVIGGYAIAKREGFDDVVVLMESNEVDHMLKGRNGHMWRDWFNDMFKKHIMKRAAKLQYGIEIAEDETVSSGPSVDNIPEYKPQPRKDITPNQDVIDAPPQQPKQDDEAAKLKAARSEVSKKFKKLGIVKEDQTEYVEKHVPGFKGTLSDFIGLSQLLDLNIEAQEAQSADGDLLD

SEQ ID NO: 130 - protein, 442 aa, Bacillus sp.
MTYAADETLVQLLLSVDGKQLLLGRGLKKGKAQYYINEVPSKAKEFEEIRDQLFDKDLFMSLFNPSYFFTLHWEKQRAMMLKYVTAPVSKEVLKNLPEAQSEVLERYLKKHSLVDLEKIHKDNKNKQDKAYISAQSRTNTLKEQLMQLTEEKLDIDSIKAELAHIDMQVIELEKQMDTAFEKNQAFNLQAQIRNLQDKIEMSKERWPSLKNEVIEDTCRTCKRPLDEDSVEAVKADKDNRIAEYKAKHNSLVSQRNELKEQLNTIEYIDVTELREQIKELDESGQPLREQVRIYSQYQNLDTQVKSAEADENGILQDLKASIFILDSIKAFRGKEAEMQAEKVQALFTTLSVRLFKQNKGDGEIKPDFEIEMNDKPYRTLSLSEGIRAGLELRDVLSQQSELVTPTFVDNAESITSFKQPNGQLIISRVVAGQELKIEAVSE

SEQ ID NO: 131 - protein, 269 aa, Shigella sonnei; identical to SEQ ID NO: 13

SEQ ID NO: 132 - protein, 648 aa, Shigella sonnei; identical to SEQ ID NO: 7

SEQ ID NO: 133 - protein, 277 aa, Salmonella enterica
MTKQPPIAKADLQKTQGNRAPAAVNDKDVLCVINSPAMKAQLAAALPRHMTAERMIRIATTEIRKVPELRNCDSTSFIGAIVQCSQLGLEPGSALGHAYLLPFGNGKAKNGKKNVQLIIGYRGMIDLARRSGQIISLSARVVRECDEFSYELGLDEKLVHRPGENEDAPITHVYAVAKLKDGGVQFEVMTKKQVEKVRDTHSKAAKNAASKGASSIWDEHFEDMAKKTVIRKLFKYLPVSIEIQRAVSMDGKEVETINPDDISVIAGEYSVIDNPEE

SEQ ID NO: 134 - protein, 648 aa, Salmonella enterica
DRGLLTKEWRKGNRVSRITRTASGANAGGGNLTDRGEGFVHDLTSLARDVATGVLARSMDVDIYNLHPAHAKRVEEIIAENKPPFSVFRDKFITMPGGLDYSRAIVVASVKEAPIGIEVIPAHVTEYLNKVLTETDHANPDPEIVDIACGRSSAPMPQRVTEEGKQDDEEKPQPSGAMADEQATAETVEPNATEHHQNTQPLDAQSQVNSVDAKYQELRAELQEARKNIPSKNPVDADKLLAASRGEFVDGISDPNDPKWVKGIQTRDSVYQNQPETEKISPDAKQPEPVVQQEPETVCNACGQTGGDNCPDCGAVMGDATYQETFGEENQVEAKEKDPEEMEGAEHPHNENAGSDPHRDCSDETGEVADPVIVEDIEPGIYYGISNENYHAGPGVSKSQLDDIADTPALYLWRKNAPVDTTKTKTLDLGTAFHCRVLEPEEFSNRFIVAPEFNRRTNAGKEEEKAFLMECASTGKTVITAEEGRKIELMYQSVMALPLGQWLVESAGHAESSIYWEDPETGILCRCRPDKIIPEFHWIMDVKTTADIQRFKTAYYDYRYHVQDAFYSDGYEAQFGVQPTFVFLVASTTVECGRYPVEIFMMGEEAKLAGQQEYHRNLRTLADCLNTDEWPAIKTLSLPRWAKEYAND

SEQ ID NO: 135 - protein, 332 aa, Acetobacter sp.
MNAPQKQNTRAAVKKISPQEFAEQFAAIIPQVKSVLPAHVTFEKFERVVRLAVRKNPDLLTCSPASLFMACIQAASDGLLPDGREGAIVSRWSSKKSCNEASWMPMVAGLMKLARNSGDIASISSQVVFEGEHFRVVLGDEERIEHERDLGKTGGKIVAAYAVARLKDGSDPIREIMSWGQIEKIRNTNKKWEWGPWKAWEDEMARKTVIRRLAKRLPMSTDKEGERLRSAIERIDSLVDISANVDAPQIAADDEFAAAAHGVEPQQIAAPDLIGRLAQMQSLEQVQDIEPQVSHAIQEADKRGDSDTANALDAALQSALSRTSTAKEEVPA

SEQ ID NO: 136 - protein, 300 aa, Acetobacter sp.
MVISKSGIYDLTNEQYHADPCPEMSLSSSGARDLLSSCPAKFIAAKQLPQQNKRCFDIGSAGHLMVLEPHLFDQKVCEIKHPDWRTKAAKEERDAAYAEGRIPLLSREVEDIRAMHSVVWRDSLGARAFSGGKAEQSLVWRDEEFGIWCRLRPDYVPNNAVRIFDYKTATNGSPDAFMKEIYNRGYHQQAAWYLDGYEAVTGHRPREFWFVVQEKTAPFLLSFFQMDEMSLEIGRTLNRQAKGIFAWCLRNNCWPGYQPEVDGKVRFFTTSPPAWLVREYEFKNEHGAYEPPEIKRKEVA

SEQ ID NO: 137 - protein, 276 aa, Salmonella enterica
MPKQPPIAKADLQKTQGARTPTAVKNNNDVISFINQPSMKEQLAAALPRHMTAERMIRIATTEIRKVPALGDCDTMSFVSAIVQCSQLGLEPGGALGHAYLLPFGNRNEKSGKKNVQLIIGYRGMIDLARRSGQIASLSARVVREGDDFSFEFGLEEKLVHRPGENEDAPVTHVYAVARLKDGGTQFEVMTRKQIELVRAQSKAGNNGPWVTHWEEMAKKTAIRRLFKYLPVSIEIQRAVSMDEKETLTIDPADASVITGEYSVVENAGVEENVTA

SEQ ID NO: 138 - protein, 268 aa, Salmonella enterica
MYYDIPNEAYHAGPGVSKSQLDDIADTPAIYLWRKNAPVDTEKTKSLDTGTAFHCRVLEPEEFSKRFIIAPEFNRRTSAGKEEEKTFLEECTRTGRTVLTAEEGRKIELMYQSVMALPLGQWLVESAGYAESSVYWEDPETGILCRCRPDKIIPEFHWIMDVKTTADIQRFRTAYYDYRYHVQDAFYSDGYRAQFGEIPTFVFLVASTTAECGRYPVEIFMMGEDAKLAGQREYRRNLQTLAECLNNDEWPAIKTLSLPRWAKENANA

SEQ ID NO: 139 - protein, 242 aa, Pseudobacteriovorax antillogorgiicola; identical to SEQ ID NO: 14

SEQ ID NO: 140 - protein, 300 aa, Pseudobacteriovorax antillogorgiicola; identical to SEQ ID NO: 8

SEQ ID NO: 141 - protein, 224 aa, Photobacterium sp.
MNTDMIAMPPSPAISMLDTSKLDVMVRAAELMSQAVVMVPDHFKGKPADCLAVVMQADQWGMNPFTVAQKTHLVSGTLGYESQLVNAVISSSKAIKGRFHYEWSDGWERLAGKVQYVKESRQRKGQQGSYQVTVAKPTWKPEDEQGLWVRCGAVLAGEKDITWGPKLYLASVLVRNSELWTTKPYQQAAYTALKDWSRLYTPAVMQGSMTGKSWSLTGRLISPR

SEQ ID NO: 142 - protein, 222 aa, Photobacterium sp.
MAERVRTYQRDAVFAHELKAEFDEAVENGKTGVTLEDQARAKRMVHEATTNPASRNWFRYDGELAACERSYFWRDEEAGLVLKARPDKEIGNNLIDVKSIEVPTDVCACDLNAYINRQIEKRGYHISAAHYLSGTGKDRFFWIFINKVKGYEWVAIVEASPLHIELGTYEVLEGLRSIASSTKEADYPAPLSHPVNERGIPQPLMSNLSTYAMKRLEQFREL

SEQ ID NO: 143 - protein, 232 aa, Providencia alcalifaciens
MKAQLAAALPKHITSDRMIRIVSTEIRKTPSLANCDIQSFIGAVVQCSQLGLEPGNALGHAYLLPFGNGKSDNGKSNVQLIIGYRGMIDLARRSGQIISISARTVRQGDNFHFEYGLNENLTHIPEGNEDSPITHVYAVARLKDEGVQFEVMTYNQIEKVRDSSKAGKNGPWVTHWEEMAKKTVIRRLFKYLPVSIEMQKAVILDEKAEANIEQDHSAIFEAEFEEVDSNGN

SEQ ID NO: 144 - protein, 277 aa, Providencia alcalifaciens
MNEGIYYDISNEDYHHGLGISKSQLDLIDESPADFIWHRDAPVDNEKTKALDFGTALHCLLLEPDEFQKRFRIAPEVNRRTNAGKEQEKEFLEMCEKENITPITNEDNRKLSLMKDSAMAHPIARWCLEAKGIAESSIYWKDKDTDILCRCRPDKLIEEHHWLVDVKSTADIQKFERSMYEYRYHVQDSFYSDGYKSLTGEMPVFVFLAVSTVINCGRYPVRVFVLDEQAKSVGRITYKQNLFTYAECLKTDEWAGIRTLSLPSWAKELKHEHTTAS

SEQ ID NO: 145 - RNA, 26 nt, Synthetic construct
  source 1..26: mol_type = other RNA
aacatgaggatcacccatgtctgcag

SEQ ID NO: 146 - RNA, 26 nt, Synthetic construct
  source 1..26: mol_type = other RNA
agcatgaggatcacccatgtctgcag

SEQ ID NO: 147 - RNA, 26 nt, Synthetic construct
  source 1..26: mol_type = other RNA
agcgtgaggatcacccatgcctgcag

SEQ ID NO: 148 - protein, 37 aa, Synthetic construct
SASGGSSGGSSGSETPGTSESATPESSGGSSGGSGGS

SEQ ID NO: 149 - protein, 22 aa, Synthetic construct
GNARTRRRERRAEKQAQWKAAN

SEQ ID NO: 150 - RNA, 15 nt, Synthetic construct
  source 1..15: mol_type = other RNA
gccctgaaaaagggc

SEQ ID NO: 151 - RNA, 15 nt, Synthetic construct
  source 1..15: mol_type = other RNA
gccctgaagaagggc

SEQ ID NO: 152 - RNA, 15 nt, Synthetic construct
  source 1..15: mol_type = other RNA
gcgctgaaaaagcgc

SEQ ID NO: 153 - RNA, 15 nt, Synthetic construct
  source 1..15: mol_type = other RNA
gccctgacaaagggc

SEQ ID NO: 154 - RNA, 15 nt, Synthetic construct
  source 1..15: mol_type = other RNA
gcgctgacaaagcgc

SEQ ID NO: 155 - protein, 21 aa, Synthetic construct
GNAKTRRHERRRKLAIERDTI

SEQ ID NO: 156 - RNA, 15 nt, Synthetic construct
  source 1..15: mol_type = other RNA
gcgctgacaaagcgc

SEQ ID NO: 157 - RNA, 15 nt, Synthetic construct
  source 1..15: mol_type = other RNA
ccgccgacaacgcgg

SEQ ID NO: 158 - RNA, 17 nt, Synthetic construct
  source 1..17: mol_type = other RNA
tgcgctgacaaagcgcg

SEQ ID NO: 159 - RNA, 17 nt, Synthetic construct
  source 1..17: mol_type = other RNA
accgccgacaacgcggt

SEQ ID NO: 160 - DNA, 200 bp, Synthetic construct
  source 1..200: mol_type = other DNA
caccttcagattttcctgtaacgatcgggaactggcatcttcagggagtagctgacctcttctcttcctcccacaggatcctggagccacccgcagttcgaaaagctcagtgaagagaagaacaaaaagcagcatattacagttagttgtcttcatcaatctttaaatatgttgtgtggtttttctctccctgtttccac

SEQ ID NO: 161 - DNA, 200 bp, Synthetic construct
  source 1..200: mol_type = other DNA
gtggaaacagggagagaaaaaccacacaacatatttaaagattgatgaagacaactaactgtaatatgctgctttttgttcttctcttcactgagcttttcgaactgcgggtggctccaggatcctgtgggaggaagagaagaggtcagctactccctgaagatgccagttcccgatcgttacaggaaaatctgaaggtg

SEQ ID NO: 162 - DNA, 121 bp, Synthetic construct
  source 1..121: mol_type = other DNA
actttgagtgtagcagagaggaaccattgccaccttcagattttcctgtaacgatcgggaactggcatcttcagggagtagctgacctcttctcttcctcccacaggatcctggagccacc

SEQ ID NO: 163 - DNA, 54 bp, Synthetic construct
  source 1..54: mol_type = other DNA
tccaccccacagtggggcaagcttctgacctcttctcttcctcccacagggcct

SEQ ID NO: 164 - DNA, 23 bp, Synthetic construct
  source 1..23: mol_type = other DNA
ttgacctgcagtccagcctangg

SEQ ID NO: 165 - DNA, 23 bp, Synthetic construct
  source 1..23: mol_type = other DNA
ccaccgcaaatgcttctaggngg

SEQ ID NO: 166 - protein, 261 aa, unidentified
  source 1..261: note = Lambda
MSTALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTREGREITGPWQSHPKRMLRHKAMIQCARLAFGFAGIYDKDEAERIVENTAYTAERQPERDITPVNDETMQEINTLLIALDKTWDDDLLPLCSQIFRRDIRASSELTQAEAVKALGFLKQKAAEQKVAA

SEQ ID NO: 167 - protein, 269 aa, unidentified; identical to SEQ ID NO: 9

SEQ ID NO: 168 - protein, 232 aa, unidentified
  source 1..232: note = T7 gp2.5
MAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEESEEADEDGDF

SEQ ID NO: 169 - protein, 270 aa, unidentified
MNQIVKFTDDSGLAVQVTPDDVRRYICENATEKEVGLFLQLCQTQRLNPFVKDAYLVKYGGAPASMITSYQVFNRRACRDANYDGIKSGVVVLRDGDVVHKRGAACYKKAGEELIGGWAEVRFKDGRETAYAEVALDDYSTGKSNWAKMPGVMIEKCAKAAAWRLAFPDTFQGMYAAEEMDQAQQPEQVRAQAEQPVDLQPIRELFKPYCEHFGITPAEGMTAVCGAVGAEGMHSMTEQQARRARAWMEEEMAAPAVEAEYEVVDEGEVF

SEQ ID NO: 170 - protein, 248 aa, unidentified
MGTALTPLLTKFATRYEMGTTPEEVANTLKQTCFKGQVNDSQMVALLIVADQYKLNPFTKELYAFPDKNNGIVPVVGVDGWARIINENPQFDGMEFSMDQQGTECTCKIYRKDRSHAISATEYMAECKRNTQPWQSHPRRMLRHKAMIQCARLAFGFAGIYDQDEAERIVERDVTPAEQYEDVSEAICLIKDSPTMEDLQAAFSNAWKAYKTKGARDQLTAAKDQRKKELLDAPIDVEFEETGDDRAA

SEQ ID NO: 171 - protein, 269 aa, unidentified
MTKQPPIAKADLQKTQGNRAPAAIKNNDVISFINQPSMKEQLAAALPRHMTAERMIRIATTEIRKVPALGNCDTMSFVSAIVQCSQLGLEPGSALGHAYLLPFGNKNEKSGKKNVQLIIGYRGMIDLARRSGQIASLSARVVREGDEFNFEFGLDEKLIHRPGENEDAPVTHVYAVARLKDGGTQFEVMTRKQIELVRSQSKAGNNGPWVTHWEEMAKKTAIRRLFKYLPVSIEIQRAVSMDEKEPLTIDPADSSVLTGEYSVIDNSEE

SEQ ID NO: 172 - protein, 267 aa, unidentified
ATNESLKNQLSTKKETGLGSAGNTIKGLMNSPAIKKRFEEVLKQRAPQYMSSIVNLVNSDINLKKCDQMSVVASCMVAATLDLPVDKNLGYAWVVPYGNKAQFQLGYKGYVQLALRTGQYKSINVIEIHEGELIDWNPLTEELKIDFSKKESDAVIGYAGYFELLNGFKKSTYWTKEQITKHKNKFSKSDFGWKKDFDAMARKTVLRNMLSKWGILSIEMQNAYTADQGIIKNEIIETGEVKENIEYIEADFESYEDNSIEEGGANE

SEQ ID NO: 173 - protein, 267 aa, unidentified
ATNESLKNQLTTKKETGLGSAGNTIKGLMNSPAIKKRFEEVLKQRAPQYMSSIVNLVNSDINLKKCDQMSVVASCMVAATLDLPVDKNLGYAWVVPYGNKAQFQLGYKGYVQLALRTGQYKSINVIEIHEGELIDWNPLTEELKIDFSKKESDAVIGYAGYFELLNGFKKSTYWTKEQITKHKNKFSKSDFGWKKDFDAMARKTVLRNMLSKWGILSIEMQNAYTADQGIIKNEIMETGEVKENIEYIEADFESYEDNSIEEGGANE

SEQ ID NO: 174 - protein, 244 aa, unidentified
ANELGIFSVDNLNMTTIKQYLDGGGKASDAELVLLINLCKQNNMNPFMKEVYFIKYGNQPAQIVVSRDFYRKRAFQNPNFVGIEVGVIVLNKDGVLEHNEGTFKTHEQELVGAWARVHLKNTEIPVYVAVSYDEYVQMKDGHPNKMWTNKPCTMLGKVAESQALRMAFPAEFSGTYGEEEYPEPEKEPREVNGVKEPDRAQIESFDKEDYAAKKIEELKEKAQPQKEVVEETGEVIDEEPLEGF

SEQ ID NO: 175 - protein, 260 aa, unidentified
STALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTREGREITGPWQSHPKRMLRHKAMIQCARLAFGFAGIYDKDEAERIVENTAYTAERQPERDITPVNDETMQEINTLLIALDKTWDDDLLPLCSQIFRRDIRASSELTQAEAVKALGFLKQKATEQKVAA

SEQ ID NO: 176 - protein, 270 aa, unidentified
STNDELKNKLANKQNGGQVASAQSLGLKGLLEAPTMRKKFESVLDKKAPQFLTSLLNLYNGDDYLQKTDPMTVVTSAMVAATLDLPIDKNLGYAWIVPYKGRAQFQLGYKGYIQLALRTGQYKSINVIEVRDGELLKWNRLTEEIELDLDNNTSEKVIGYCGYFQLINGFEKTVYWTRKEIEAHKKKFSKSDFGWKKDYDAMAKKTVLRNMLSKWGILSIDMQTAVTEDEAEPRERKDVTEDESIPDIIDAPITPSDTLEAGSEVQGSMI

SEQ ID NO: 177 - protein, 260 aa, unidentified
STALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTREGREITGPWQSHPKRMLRHKAMIQCARLAFGFAGIYDKDEAERIVENTAYTAERQPERDITPVNDETMQEINTLLIALDKTWDDDLLPLCSQIFRRDIRASSELTQAEAVKVLGFLKQKASEQKVAA

SEQ ID NO: 178 - protein, 271 aa, unidentified
  source 1..271: note = Gammaproteobacteria
EKPKLIQRFAERFSVDPNKLFDTLKATAFKQRDGSAPTNEQMMALLVVADQYGLNPFTKEIFAFPDKQAGIIPVVGVDGWSRIINQHDQFDGMEFKTSENKVSLDGAKECPEWMECIIYRRDRSHPVKITEYLDEVYRPPFEGNGKNGPYRVDGPWQTHTKRMLRHKSMIQCSRIAFGFVGIFDQDEAERIIEGQATHVVEPSVIPPEQVDDRTRGLVYKLIERAEASNAWNSALEYANEHFQGVELTFAKQEIFNAQQQAAKALTQPLAS

SEQ ID NO: 179 - protein, 260 aa, Escherichia coli
STALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTREGREITGPWQSHPKRMLRHKAMIQCARLAFGFAGIYDKDEAERIVENTAYTAERQPERDITPVNNETMQEINTLLIALDKTWDDDLLPLCSQIFRRDIRASSELTQAEAVKALGFLKQKATEQKVAA

SEQ ID NO: 180 - protein, 244 aa, Lactococcus lactis
ANEIDIYDAKNLNTATVKKFLKGGGQASDEELAMLLAISRNQNMNPFMKEVYFIKYGSAAAQIVVSRDFYRKRAFQNPNFAGIEVGVIVLNKDGVLEHNEGTFKTKDQELVGAWARVHLKNTEIPVYVAVSYDEYVQMKNGQPNSMWTNKPCTMLGKVAESQALRMAFPAEFSGTYGEEEYPEPEKEPREVNGVKEPDRAQIESFDKENYAARKIEELKEKAQPQKEFVEEIGEAIDEITAEDF

SEQ ID NO: 181 - protein, 260 aa, unidentified
STALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTREGREITGPWQSHPKRMLRHKAMIQCARLAFGFAGIYDKDEAERIVENTAYTAERQPERDITPVNDETMQEINTLLIALDKTWDDDLLPLCSQIFRRDIRASSELTQAEAVKALGFLKQKASEQKVAA

SEQ ID NO: 182 - protein, 244 aa, unidentified
ANELGIFSVDNLNMTTIKQYLDGGGKASDAELVLLINLCKQNNMNPFMKEVYFIKYGNQPAQIVVSRDFYRKRAFQNPNFVGIEVGVIVLNKDGVLEHNEGTFKTHEQELVGAWARVHLKNTEIPVYVAVSYDEYVQMKDGHPNKMWTNKPCTMLGKVAESQALRMAFPAEFSGTYGEEEFPEPEKEPREVNGVKEPDRAQIESFDKEDYAAKKIEELKEKAQPQKEVVEETGEVIDEEPLEGF

SEQ ID NO: 183 - protein, 263 aa, unidentified
TTALQTLTNKLAERFEMGDGSGLVETLKSSAFAGATVSDAQMIALLVIANQYQLNPWTKEIYAFPGGNGGLTPIVGVDGWVRIINREPQYDGMEFHFTDDYSACTCTIYRKDRSKPIVVTEFMGECKKSSPAWNSHPKRMLRHKAMIQCARLAFGFTGIYDQDEAERIAENEKPPKNITPQNNVVETTAVELISEEQLSQIRQLMQVTGTEEAKILAYIGVQALNQIPKSQAEAVIKKLNLTLDKQNAEKADNGESVGEEIPL

SEQ ID NO: 184 - protein, 247 aa, unidentified
AKNELAKGSYLTDLQKLDGNTLRDFVDPKHQASPQELQALLAIVKGRNLNPFTKEVYFIKYGSAPAQIVVSKEAIMKRAEENPDFDGFEAGIVVETKDGAIERLTGTIVPKSATLRGGWCKVYRKDRSHAIEADADFAYYTTSKNLWQKMPALMIRKVAIVSAFREAFSESVGGLYTADEMQRETQAEVRARKMKEAYEEKLYLLTQMEAKSYKKTKSKNENEAKKTKEAEAIETVEEPTQDGNLEW

SEQ ID NO: 185 - protein, 260 aa, unidentified
MAKENYSDPNGKLLNSITTFEVNGEEVKLSGNIIRDYLVSGNAEVTDQEIIMFLQLCKYQKLNPFLNEAYLVKFKNTKGPDKPAQIIVSKEAFMKRAETHEQYDGFEAGVIVERGGEIIELEGAVSLASDKLLGGWAKVFRKDRNRPVSVRISEKEFNKRQSTWNTMPLTMMRKTAVVNAMREAFPDNLGAMYTEEEQGSLQNTETSVQQEIKQNANAEVLDIPSQQNEVPDFKEVREPEHVEMPPIYGEQQSTPPARPY

SEQ ID NO: 186 - protein, 270 aa, unidentified
ATNDELKNQLANKQNGGQVASAQSLDLKGLLEAPTMRKKFEKVLDKKAPQFLTSLLNLYNGDDYLQKTDPMTVVTSAMVAATLDLPIDKNLGYAWIVPYKGRAQFQLGYKGYIQLALRTGQYKSINVIEVRDGELLKWNRLTEEIELDLDNNTSEKVIGYCGYFQLINGFEKTVYWTRKEIEAHKKKFSKSDFGWKKDYDAMAKKTVLRNMLSKWGILSIDMQTAVTEDEAEPRERKDVTEDESIPDIIDAPITPSDTLEAGSEVQGSMI

SEQ ID NO: 187 - protein, 223 aa, unidentified
PQEIAKVEYTAADGQEVRLTPGVIAKYIVSGNGLASEKDIYSFMARCQARGLNPLAGDAYMTVYQGKDGNTSSSVIVSKDYFVRTATAQDSFDGMEAGVTVLNGQGQIQKREGCEFFPSLGEKLLGGWAKVHVKDREHPSKAAVTMDEYDQHRSLWKSKPATMIRKVAIVQALREAYPGQFGGVYDRDEMPPSQEPQQVPVEVYEAPEAYETPDNQNRATEEF

SEQ ID NO: 188 - protein, 266 aa, unidentified
NTPTMKRKFEEVLHENANAFMSNVMTLVSNDSYLAESEPMSILSGALTAATLNLGLDKNLGYAYLVPFNTKNKQTGKWERKAQFILGYKGYIQLAQRSGKYKALNVIEVYEGELLSWNRLTEEFEFDPNGRQSDDVIGYVGYFELLNGFKKTVYWTKQEIEAHRIANSKDKEKTKLSGVWATDYNAMARKTVLRNMLSKWGILSIEMQEATTSDEKVQQMQEDGNIISETEVEENTTMKTAEVINEADSDSLNQTDLFDTKNPPLE

SEQ ID NO: 189 - protein, 267 aa, unidentified
  source 1..267: note = butyrate-producing bacterium SS3/4
ENATAVQQAESQGTQDFSAPVKHNTDFSLGIFGSSDNFLMATQMAKAFASSTIVPKEYQGNFANGLVAMDIANRLKTSPFMVMQNLDVIQGRPAWRATFLIAMINRSKKYDIELQFEEKRDKNGKPYSCTCWTTKDGRKVTGIEVTMDMAEAEGWTKKNGSKWITMPQVMLRYRAASFFSRMNCPELSNGLYTTDEVYEMADSEYKVYNLEDEVKRDLAQNANKEEFVAPPNETAPESESKGSEPLDPAVENQKSGDTPDWMKPETM

SEQ ID NO: 190 - protein, 263 aa, Cellulosilyticum lentocellum
SDKKELVLKETHSRLNQLLATKMEAMPKDFNQTRFLQNCMTVLQDTKGIENCHPVSIARTLLKGAFLGLDFFQRECYAIPYGGELQFQTDYKGETKMAKKYSIRDIKDIYAKVVRKGDEFKEEIVAGQQVVDFKPLPFNDAEIIGAFAVVLYQDGGMEYETMSTKQIEGIRDNFSKMKNGLMWTKTPEEAYKKTVLRRLTKKIEKDFASIDQAKAYEESSDMQFKQDEQKQDAKDPFADAVDVEFTEETEGQVRLDGEADGAK

SEQ ID NO: 191 - protein, 262 aa, unidentified
SNELMTKAVTYEVNNEEVKLSGQIVKQYLTSGQAVTDQEVTMFIQLCRYQHLNPFLNEAYLVKFNGKPAQIITSKEAFMKRAESNPNYAGLKAGCIVERNGELIYTEGAFTLKTDNILGAWADVIRKDRREPTHVEISMDEFSKSQATWKSMPATMIRKTAIVNALREAFPQDLGALYTEDDKNPNEATQTTYKQEPEVNTTKTADVLAKKFSGAPQIKSVENVQESEEESNNASNHGEATEPVNNVEEPTATAEVEQGQLL

SEQ ID NO: 192 - protein, 253 aa, unidentified
TNNQLATQIKRDITTDPSLLTGADIKKYFDPQNLLTEKQVGQALALCKGRNLNPFANEVYIVAYQNNSGTEFSLIVSKEAFMKRAERCEGYDGFEAGITVMRNGEMIEIEGSLKLPEDILIGGWAVVYRKDRSHRYKVTVDFNEYVKTDRNGNPRSTWKSMPATMIRKTALVQTLREAFPDELGNMYTDIDGGDTFDAIKDVTPQESREDVVARKMAQIDQFNKEQEANHADPEPTQNEDPIQGELLDGELEY

SEQ ID NO: 193 - protein, 271 aa, unidentified
TNNQIVEAKGDFLTNPQLLNSGIIRKYLDPQGKASDEELAYFIAQAKAQNLNPFTKEIYFIKYGTQPAQIVTAKSAFEKKADSHPQFDGKEAGVIYLMDGEIKYSKGAFIPKGAEILGGWAKVYRKDRTYPTETEVSFEEYDNSKIRARVKELTQQGKDVTYPVMNSYGKPIGENNWDTMPCVMIRKVALVSAYREAFPAELGASYEADEIQLDNTPKDVTPQESREDVVARKMAEIEQFNKEQEANHADPEPTQNEDPIQGELLDGELEY

SEQ ID NO: 194 - protein, 241 aa, unidentified
NNEVMEKSVEYEVNGNSVKLTPNMIKQFITKGNADVTDQEAIMFMKLAEQQQLNPFLNEVYLIKFKGKPAQNIVAKEAFMKRAEKHSEYDGLEAGIIVQRGEEIKELPGAVCLPTDNLLGGWARVYRKDRKNPFYVQLDFKEFSKGQATWNQMPKNMIRKTAIVNALREAFPEALGAMYTEDDARLEEVKTAEPIKEKAETTQILENKFKELSENGQTEVGDEQTNESTEPEPTAKQEQLL

SEQ ID NO: 195 - protein, 205 aa, unidentified
TVQLVQPRNSDEYDFDQTKLDLIKRTICKGATNDELQLFIHACKRTGLDPFMRQIFAVKRWDSSTKKEIMTIQTGIDGYRLIADRTGKYAPGKDTEFGYDNKGNIRWAKAYIKKMTPDGQWHEISAIAFWEEYVQTTREGKSTLFWLKKSHIMLSKCTEALALRKTFPAERSGIYTKEEMAQEFSPLEEHLVERIAASRNDQGRS

SEQ ID NO: 196 - protein, 255 aa, unidentified
SNNQLSTQQAKRDIAIDTSVWTFQDVKRYFDPQNLLTEKQVGQALSLIKGRNLNPLANEVYIVAYKKKTGGTEFSLIVSKEAFLKRAAQNPNYEGFEAGVVTVDTDGVMHERKGALMLPGDTLVGGWARVYRKNFKVPVEIFVSREEYDKKQSTWNAMPATMIRKTALVNALREAFPEDLGNMYTEDDGGETFDRIKQAEPVESREDVMARKMAQIEQMKQEQAQRQIDTSYPTDDVIDPDDEPAQGELLEDLEY

SEQ ID NO: 197 - protein, 258 aa, unidentified
AITPNPIPAQDGSPIPSPDDIVGELARRKIYAGIPDDDVALALALCQKYGFDPLLKHLVLLATKDRDETTGQGQKHYNAYVTRDGLLHVAHTSGMLDGLETIQGKDDLGEWAEAVVYRKDMSRPFRYRVYLSEYVREAKGVWKTHPQAMLTKTAEVFALRRAFDVALTPFEEMGFDNQNIAGDTGPSPKTGFTEKAGFTGNTDFSAEASLPGKARFSTEAGLTDMTVIPPNRVTGSIPETSRLNTSAGSTGRQRRQLF

SEQ ID NO: 198 - protein, 260 aa, unidentified
STALATLAGKLAERVGMDSVDPQELITTLRQTAFKGDASDAQFIALLIVANQYGLNPWTKEIYAFPDKQNGIVPVVGVDGWSRIINENQQFDGMDFEQDDESCTCRIYRKDRNHPICVTEWMDECRRAPFKTREGREITGPWQSHPKRMLRHKAMIQCARLAFGFAGIYDKDEAERIVENTAYTTERQPERDITPVNEETMSEINALLTSMEKTWDDDLLPLCSQIFRRYIRASSELSQAEAEKVLGFLKQKATEQKVAA

SEQ ID NO: 199 - protein, 271 aa, unidentified
EKPKLIQRFAERFSVDPNKLFDTLKATAFKQRDGSAPTNEQMMALLVVADQYGLNPFTKEIFAFPDKQAGIIPVVGVDGWSRIINQHDQFDGMEFKTSETKVSLDGAKECPEWMECIIYRRDRSHPVKITEYLDEVYRPPFEGNGKNGPYRVDGPWQTHTKRMLRHKSMIQCSRIAFGFVGIFDQDEAERIIEGQATHVVEPSVIPPEQVDDRTRGLVYKLIERAEASNAWNSALEYANEHFQGVELTFAKQEIFNAQQQAAKALTQPLAS

SEQ ID NO: 200 - protein, 270 aa, unidentified
TTVNQTELKNKLAEKAKTPAKTGNTVFDLIRKMEPEIKRALPKQISPERFARIAMTAVRNTPKLQACEPISFIAALMQSAQLGLEPNTPLGQAYLIPYGKEVQFQLGYQGMLTLAYRTGEYQSIYAMPVYANDEFEYEYGLNEKLVHKPAPDPEGEPIYYYAVYKLKNGGHGFVVMSRQQIERHRDKYSPSAKQGKFSPWNTDFDSMAKKTVLKQLLKYAPKSVEFATQIAQDETIKTEIAEDMTEVQGIEVEYEATDDQENQENQEQED

SEQ ID NO: 201 - protein, 266 aa, unidentified
EFETDEEEKEMSNNQLSTQQAKRDIAIDTSVWTFQDVKRYFDPQNLLTEKQVGQALSLIKGRNLNPLANEVYIVAYKKKTGGTEFSLIVSKEAFLKRAAQNPNYEGFEAGVVTVDTDGVMHERKGALMLPGDTLVGGWARVYRKNFKVPVEIFVSREEYDKKQSTWNAMPATMIRKTALVNALREAFPEDLGNMYTEDDGGETFDRIKQAEPVESREDVMARKMAQIEQMKQEQAQRQIDTSYPTDDVIDPDDEPAQGELLEDLEY

SEQ ID NO: 202 - protein, 271 aa, unidentified
TTNQVVTHKNFFNAPNVQKSFDDVWKGAGVQFATSILSVIQGNASLKSASNESIMTSAMKAAVLNLPIEPSLGRAYLVPYKGQVQFQLGYKGLIELAQRSGKYKSINAGPVYKSQFVSYDPLFEELTLDFTQPQDEVIGYFASFSLLNGFRKLTYWTKAEVEAHGKKFSKTFGNGPWKTDFDAMARKTVLKHILSIYGPLSVEMQTGMQNDESENDNATRDIKTAEPVNADQQLLEDLMNVDTETGEILEEVSELKDNGELDLKYEDPNAR

SEQ ID NO: 203 - protein, 247 aa, Pseudomonas aeruginosa
GTALTPLLTKFATRYEMGTTPEEVANTLKQTCFKGQVNDSQMVALLIVADQYKLNPFTKELYAFPDKNNGIVPVVGVDGWARIINENPQFDGMEFSMDQQGTECTCKIYRKDRSHAISATEYMAECKRNTQPWQSHPRRMLRHKAMIQCARLAFGFAGIYDQDEAERIVERDVTPAEQYEDVSEAICLIKDSPTMEDLQSAFSNAWKAYKTKGARDQLTAAKDQRKKELLDAPIDVEFEETGDDRAA

SEQ ID NO: 204 - protein, 247 aa, Pseudomonas aeruginosa
GTALTPLLTKFATRYEMGTTPEEVANTLKQTCFKGQVNDSQMVALLIVADQYKLNPFTKELYAFPDKNNGIVPVVGVDGWARIINENPQFDGMEFSMDQQGTECTCKIYRKDRSHAISATEYMAECKRNTQPWQSHPRRMLRHKAMIQCARLAFGFAGIYDQDEAERIVERDVTPAEQYEDVSEAVCLIKDSPTMEDLQSAFSNAWKAYKTKGARDQLTAAKDQRKKELLDAPIDVEFEETGDDRAA

SEQ ID NO: 205 - protein, 242 aa, unidentified
SSIAAAAESAEVTPASIINKYRDDIATVLPPKLRERIDRWIRLAIGAVNSNPELISRVRADQGASMMQALMKCAALGHEPGSGLFHLVPKGSRIEGWEDYKGILQRIDRSGVYARTVIGVVYANDEYSYDQNVDERPRHVRATGDRGEPISSYAYAVYPSGAITTVAEATPEQIASSKSKARGADNAASPWRAPGAPMHRKVAVRLLEKHVATSAEDRREPISRSAANDVVIDATADYYQEP

SEQ ID NO: 206 - protein, 245 aa, unidentified
TNELTQTKGAYLTDLQKLDGATLRNFVDPKHQASPQELQTLLAIVKNRNLNPFTKEVYFIKYGNNPAQIVVSKDAFMKRAEQNQNYDGFESGIIYEDASGELKNKKGVILPKNCTLIGGWCEVYRKDRTRPVYREVELSAYNTGKNWWQKAPGQMIEKVAIVAAVRDSFSEDVGGLYTSEEMEQAAPIDVTPQESQEEVRTRKMAQIEEMKREQEKHQSSAYPEDEIPNFEDEPLQGELLEEMEY

SEQ ID NO: 207 - protein, 268 aa, unidentified
NERTNLQYAPAPVERFKECLNSHEIKARLKNSLKNNWTQFQTSMLDLYSGDAYLQKCDPMAVALECVKAATLDLPISKSLGFAYVVPYNNVPTFTLGYKGLIQLAQRTGQYRTINADVVYEGEIRGADKLSGMVDLSGERTGDEVVGYFAYFKLINGFEKMIYMTRAEAEKWRDDYSPSAKSKYSPWRTDFDKMALKTCIRRLISKYGIMSVEMQGVMTEEAEPRAAAAAKRAEETVQANANSKVIDIDAAPPAANESPAEAAPQPDF

SEQ ID NO: 208 - protein, 250 aa, unidentified
DYVTKIQEVLNRLLDAKHDALPSGFKKTRFSENCRAYVKEYTDLQKYDEEEVALVLFKGAVLGLDFLAKECHVITEGSALRFQTDYKGEMALVKKYSVRPILDIYAKNVREGDVFREEISEGKPLIHFNPLAFNNSQIIGSFAVALFSDGGMVYETMPAEEIESIRRNYGKNPGSDTWEKSQGEMYKRTVLRRLCKTIEIDFDAEQSLAYEAGSSFEFNREPQPKKRSPFNPPEVEESEVLSDDGTSEAE

SEQ ID NO: 209 - protein, 259 aa, unidentified
SNALTITQDQTEFTPKQLSVLENLGVQGAAPQEVAMFFDYCQRTGLSPWARQIYMIGRWDRNLGRKKYAVQVSIDGQRLVAERSGVYEGQTAPQWCGPDGQWVDVWLANEPPQAARVGVWRKSFREPAYGVARLSSYMPVTRDGKPQGLWGTMPDVMLAKCAESLALRKAFPLELSGLYTSEEMQQADAPRTEPAPVDEDVVDAEIVDDEERMQWVEAIQAAETTDVLRKMWADIKTCPDALQAELRELIPARAKELAA

SEQ ID NO: 210 - protein, 171 aa, unidentified
IECAKLGLEPNNILGQAYLVPVCVDGVNKVEFQLGYKGLIELAYRSGKIKSLYANEVFEKDEFHIDYGLDQKLIHKPFLGGDRGEVIGYYAVYQMDNRGASFVFMTRDEILGHSRKYSRSFGCDLWESEFDAMAKKTVIKKLLKYAPLSIELQKSVSVDESVKGIGCIGVI

SEQ ID NO: 211 - protein, 247 aa, unidentified
GTALTPLLTKFATRYEMGTTPEEVANTLKQTCFKGQVNDSQMVALLIVADQYKLNPFTKELYAFPDKNNGIVPVVGVDGWARIINENPQFDGMEFSMDQQGTECTCKIYRKDRSHAISATEYMAECKRNTQPWQSHPRRMLRHKAMIQCARLAFGFAGIYDQDEAERIVERDVTPGEPVEDVTEALSLINSAPTMDDLQAAFSDAWKAYKSKGARDQLTVAKDQRKKELLEAPIDVEFEETGDDRAA

SEQ ID NO: 212 - protein, 260 aa, unidentified
STALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTREGKEIIGPWQSHPKRMLRHKAMIQCARLAFGFAGIYDKDEAERIVENTACTAEHQPERDITPVNDETMQEINTLLIALDKTWDDDLLPLCSQIFRRDIRASSELTQAEAVKALGFLKQKATEQKVAA

SEQ ID NO: 213 - protein, 249 aa, unidentified
TNNQLATQTKRNITTDPSLLTGADIKKYFDPQNLLTEKQVGQALALCKGRNLNPFANEVYIVAYTNRNGGKEFSLIVSKEAFLKRAAQCKDYEGFEAGVVVVDSEGVMHERKGAIMLPEDTLIGGWARVHRKNFKVPVEIFVSREEYDKKQSTWNTMPATMIRKVALVNALREAFPEDLGNMYTEDDGGETFDRIKDVTPQESREDVVARKMAQIEQFNKEQEANHADPEPAQTEETIQGELLDGELEY

SEQ ID NO: 214 - protein, 158 aa, unidentified
SNREIEVIRACSKAGNNGGSSPWDSFPDEMARKAIVKRASKYWPRRDRLDTAIDYLNTQGGEGIILNADHIPERDVTPASDEIINEITQAITEINKTWDDLLPLCSKTFRRTIASHEYLSQEEAVKTLDFVKKKAARNKATAEAKIHATTENNSEAVS

SEQ ID NO: 215 - protein, 270 aa, unidentified
ATNDELKNKLANKQNGGQVASAQSLDLKGLLEAPTMRKKFEKVLDKKAPQFLTSLLNLYNGDDYLQKTDPMTVVTSAMVAATLDLPIDKNLGYAWIVPYKGRAQFQLGYKGYIQLALRTGQYKSINVIEVREGELLKWNRLTEEIELDLDNNTSEKVVGYCGYFQLINGFEKTVYWTRKEIEAHKKKFSKSDFGWKKDYDAMAKKTVLRNMLSKWGILSIDMQTAVTEDEAEPRERKDVTDDESIPDIIEAPITPSDTLEAGSVVQGSMI

SEQ ID NO: 216 - protein, 249 aa, unidentified
TNNQLATQTKRNITTDPSLLTGADIKKYFDPQNLLTEKQVGQALALCKGRNLNPFANEVYIVAYTNRNGGKEFSLIVSKEAFLKRAAQCKDYEGFEAGVVVVDSEGVMHERKGAIMLPEDTLIGGWARVHRKNFKVPVEIFVSREEYDKKQSTWNTMPATMIRKVALVNALREAFPEDLGNMYTEDDGGETFDRIKDVTPQESREDVVARKMAQIEQFNKEQEANHADPEPAQTEEPIQGELLDGELEY

SEQ ID NO: 217 - protein, 249 aa, unidentified
TNNQLATQTKRNITTDPSLLTGADIKKYFDPQNLLTEKQVGQALALCKGRNLNPFANEVYIVAYTNRNGGKEFSLIVSKEAFLKRAAQCKDYEGFEAGVVVVDSEGVMHERKGAIMLPEDTLIGGWARVHRKNFKVPVEIFVSREEYDKKQSTWNTMPATMIRKVALVNALREAFPEDLGNMYTEDDGGETFDRIKDVTPQESREDVVARKMAEIEQFNKEQEANHADPEPAQTEESIQGELLDGELEY

SEQ ID NO: 218 - protein, 249 aa, unidentified
TNNQLATQTKRNITTDPSLLTGADIKKYFDPQNLLTEKQVGQALALCKGRNLNPFANEVYIVAYTNRNGGKEFSLIVSKEAFLKRAAQCKDYEGFEAGVVVVDSEGVMHERKGAIMLPEDTLIGGWARVHRKNFKVPVEIVVSREEYDKKQSTWNTMPATMIRKVALVNALREAFPEDLGNMYTEDDGGETFDRIKDVTPQESREDVVARKMAEIEQFNKEQETNHADPEPAQTEETIQGELLDGELEY

SEQ ID NO: 219 - protein, 259 aa, unidentified
NEIQAYDKINDRDGMEMLGAAIQRSGMFGAETKEQGIILALQCMVEKKPPLEMAKNYHIIQGKLSKRADAMLADFRKAGGKFIFADLKNPTVQKAKVTFEDYKDFDVEYSIDDAKTAGVYNAKGAWVKYPGAMLRARLVSETLRAIAPEIVTGVYTPEELETPINAKPELKCAQPVKAKPEPKKAQPDVIEATVCESELDAKLVELIGDREQIVNLYWEKKGLIDGLDTTWRDLNDDTKRKMIDQFDQFMDAAQRKAAQ

SEQ ID NO: 220 - protein, 218 aa, unidentified
AENEKQALLQEENKSENVVSTVKRTALATNPFSDTDQFNNIFKMAQLISQSDMIPATYKGKPMNCVIALEQANRMGVSPLMVMQNLYVVKGVPSWSGQGCMMIIQGCGKFRDVDYVYSGEKGTDSRSCKVVATRISDGKRIEGTEITMQMVKSEGWISNTKWKNMPEQMLGYRAATFFARMYCPNELNGFATEGEAEDMNHKPQRIEAINVLGDTAHE

SEQ ID NO: 221 - protein, 270 aa, unidentified
TIMDLLNDPKMKSQIQRALPNGMSAERIARIALTALRMNPQLQECSPQSFAAALMTSAQLGLEPNTPLGHAWLIPRKNHGKMEVQFELGYKGMLDLVRRSGMITAIFAEEVREKDEFEFEYGTNPYLKHKPYLGGDRGKVLFYYAVATFKDGGYAFKVMSIPEIEEARKLSQSANSPYSPWNRFYDEMAKKTVLKRLCKYLPLSIEVQRNLAQDETIRTQIEADDILDLPNENEFEVVEVEEIPGEEEKEEAKEGPFPNKALRESPTPLT

SEQ ID NO: 222 - protein, 244 aa, unidentified
STALTTLTSQLSQRFKLDGGEELLTTLKQTAFKGQVTDAQMTALLIVANQFGLNPWTKEIYAFPDKNGGIVPVVGVDGWARIINEHPQFDGMDFEMDGEQSCTCVIYRKDRTRPIRITEYMAECKKTGGGPWQSHPRRMLRHKAMIQCARMAFGFGGISDEDDAERIREKDITPQAEVVPKALEPYPADKFEENFEQWKSLIESGRRSADDVIAKIKSRNTMTDEQETRLRACGGEEGKTYENA

SEQ ID NO: 223 - protein, 244 aa, Pseudomonas sp.
SNVATIKPSSLSARMAERFGVDPNEMMATLKATAFKGQVSDAQMQALLIVADQYGLNPWTKEIYAFPDKGGIVPVVGVDGWSRIINENGAFDGMDFQQDDESCTCIIYRKDRNHPIKVTEWMAECKRNTQPWQSHPKRMLRHKAMIQCARLAFGYTGIFDEDEAQRIVEKDVTPAVNEPDITPALEAIKNASSMEELHAAFKAAWNQHPSARARLTAVKDERKKALSEPIEGELVENEDGPAQQ

SEQ ID NO: 224 - protein, 261 aa, Streptococcus sp.
AKNELVKGEYLTDLQKLDGNTLRNFVDPKHRASPQELQALLAIVKNRNLNPFTKEVYFIKYGSAPAQIVVSKEAIMKRAEENPNFDGFEAGIVIETKSGSIERLTGTIAPKRAELRGGWCKVYRKDRSHAIEADADFAYYTTGKNLWQKMPALMIRKVAIVSAFREAFSESVGGLYTADEMEQNNTQETQEEVRARRMKQAYEEKLRLLTEMEAKSYKKVEDESASKEIEAAKTTKNTKEVEVIEETEVTEEPTQEDSLEW

SEQ ID NO: 225 - protein, 260 aa, unidentified
STALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTRDGREITGPWQSHPKRMLRHKAMIQCARLAFGFAGIYDKDEAERIVENTTYTTDRQPERDITPVSDETMREINDLLITMNKTWDDDLLPLCSQIFRRDIGASSDLTQIEAVKALGFLKQKAAEQKVEA

SEQ ID NO: 226 - protein, 262 aa, Pirellula sp.
SEATKEAKPETAIAKKPNGIKDWLKSDALKTQIASVAPKHMAPERVMRIALNAVSRTPKLADCTPESFMRCLLDLSSWGLVPDGRHAHLIPYGTECTLVLDYKGLVTLAYRSGWVKKIHADVVFEGDIFVYSLGTVCQHIPWEFRDDANKPEHKGHFRAAYCVVTMADGIEKHEVMTASEIDAIKAKSRSGNSGPWKDHYTEMAKKTVYRRASKWLPLSPEQADAMERDDDRIIDAVSVAVTQRLSKAAMPLIGANETGDTE

SEQ ID NO: 227 - protein, 262 aa, Pirellula sp.
SEATKEAKPETAIAKKPNGIKDWLKSDALKTQIASVAPKHMAPERVMRIALNAVSRTPKLADCTPESFMRCLLDLSSWGLEPDGRHAHLIPYGTECTLVLDYKGLVTLAYRSGWVKKIHADVVFEGDIFVYSLGTVCQHIPWEFRDDANKPEHKGHFRAAYCVVTMADGIEKHEVMTASEIDAIKAKSRSGNSGPWKDHYTEMAKKTVYRRASKWLPLSPEQADAMERDDDRIIDAVSVAVTQRLSKAAMPLIGANETGDTE

SEQ ID NO: 228 - protein, 229 aa, unidentified
SEIQQQAEAQTQAHPTAVLDDYRGAIASVAPPGTNIDLFIRMTKSNVNRSDEIVAAVKRNPGLFMQAVMDSAALGHIPGSEYYYLTPRRDGISGIESWKGVAKRIFNTGRYQRIVCEVVYEGEQWEFQPGEDLKPKHVIDWDARQVGSKVRFTYAYAVDFEGNPSTVAVCTKLDLDKAQKQSRGKVWDQWYEQMAKKTAIKRLEDFVDTSAVDLRADGSSRRHSAEVAE

SEQ ID NO: 229 - protein, 227 aa, unidentified
ASKNEAIEVSPAEIASVKEKPASIVKAEKAKKEPCALVKYEDAEGREVVLTREDIINTISSNPRITDKEIKLFIELARAQKLNPFTREIFITKYGDYPATFIVGKDVFTKRAQSNPLFKGMQAGIIVQRGNAVDQREGSATFGDEMLIGGWCKVYVQGYDVPIYDSVSFNEYAARKTDGTLNAMWASKPATMIRKVAIVHALREAFPSDFQGLYDQSEMGLSGQGGE

SEQ ID NO: 230 - protein, 258 aa, unidentified
ATSLKRAVTGDGKPATVQQLLTNPKIKSQIALALPQHLTPERLTRIVLTEIRRTPALAKCKPESLLAAVMQCAQLGLEPGGSLGHAWLMPFKNEVQFIIGYRGMIDLARRSGQVLSIEARGVYESDTFHVSFGLEPDLTHQPDWDPADRGKLAFVYAVARLKDGGFQFDVMSRAEVEKIRAQSPAGKSGPWVTHFEEMAKKTVIRRLFKYLPVSVEMARAVGLDEAAERGEQSDAIDADCVIESEEEATPEEKGDSAA

SEQ ID NO: 231 - protein, 256 aa, unidentified
SEISKAVATQQNPLAVVARYKRELGTVLPTVLRQDPDRWLMAAENAARKNPDIMAVTKADQGASYMRALVECARLGHEPGSKDFHFIKRGNAISGEESYRGIIKRVLNSGFYRSVVARTVFSNDTYSFDPLTDIVPNHVPAQGDRGKPLSAYAFAVHWDGTPSTVAEATPERIATAKAKSFASDKPTSPWQLPTGVMYRKTAIRELEPYVHVAPEPQPRRHLDGTVGGIPATDFDVDDGDVLDITADQLAEAGEIV

SEQ ID NO: 232 - protein, 240 aa, unidentified
SELQQAAQGQADAGPVQVIYSHAKEIQNVLAKGTDMDRWLQMARLAVMRDPNLVNAAKRDPGSLMQAMLDCAEKGHIPGTEDYYLVPRKGGIQGMESWKGIAKRIMRSGRYQSIVAEVVYEGEDFDFNPNTMDRPVHQIKYMARTSGQPVLSYAYAVDHEGKPSTIAVADPRYIAKVKANSKGTVWADWDEAMYKKTAVKMLVDYVDTSSTDRRGVSTVQVDGPVGTFIDGVLEIEGGDQ

SEQ ID NO: 233 - protein, 240 aa, unidentified
SELQQAAQGQQSNNPVSFIYSHAKDIQNVLTKGTDMDRWLQMARLAVMRDQNLVASAKRDPGSLMQALLDCAEKGHVPGTEDYYLVPRKGGIQGMESWKGIAKRIMRSGRYQSIVNEVVYEGETFEFNPNTMDRPVHNINYMTRTSGKPVMSYAYALDHDGKPSSVAIADPRYIAKVKKNSRGSVWEDWDEQMYRKTAVKMLQDYVDTSSVDRRDVSTVQVDDANVIDVDDTFTAREAGE

SEQ ID NO: 234 - protein, 254 aa, unidentified
TQDLATAIADQQPAQRRTAFDLVESMRGELHKALPEHASIDNFLRLALTELKMNPQLGNCSGESLLGALMTAARVGLEVGGPLGQFYLTPRRLKRDGWAVVPIVGYRGLITLARRAGVGQVNAVVVHEGDTFREGASSERGFFFDWEPAVERGKPVGALAAARLAGGDVQHRYLSLAEVHERRDRGGFKDGSNSPWATDYDAMVRKTALRALVPLLPQSTALSFAVQADEQVQRYDAGDIDIPALDETDTEDTK

SEQ ID NO: 235 - protein, 248 aa, unidentified
TNQLAHKDFFNTPAVKQKFQEVLNGNERQFTASLLSIVNNNKLLARASNTSIMTAAMKAAVLNLPIEPSLGFAYIVPYGQDAQFQLGYKGLIQLAIRSGQFKAINSGKVYKAQFKSYDPLFETLDIDFTQPEDEVYGYFATFELVNGFKKLTFWTKEQAESHGKRFSKTYAKGPWSTDFDAMAQKTVLKSILSKYAPLSTEMQEGLISDNQTEEVETDPIDVTPKNEDTQTLLSDLMSDEAESETEKV

SEQ ID NO: 236 - protein, 264 aa, unidentified
TSQQLDTTHTINQQVTTFRHTLVQMKNEIAAALPAHMTGDRFLRLILTEVRKNPELAECSTESIFGGILTAAALGLEPGLNGECWLIPRKVGKGPGSRKEATFQVGYKGIIKLFWQNPLASYLDTGVVYANDAWKFRKGLDPILEHTPATGDRGAVRGYYAVVGLTTGARIFDFFTPKQISALRGTAGPNGGISDPEHWMERKTALLQVMKMAPKSTDLASAASVDGTVQTVEAAAQVAAASTGPVNPTTGEVLEAEPVEGGAA

SEQ ID NO: 237 - protein, 264 aa, unidentified
TQQMPIEAQGEPTKELQQKAAVDRFNATLHQMQNEIARALPKHMTGDRFVRIVLTEVRKDPTLALCDPLTMFGSLLTAAALGLEPGLNGECWLVPRKNHGTLEAQLQVGYRGVVKLFWQNPAAAYLDTGYVCERDHFRFAKGLNPILEHTPAEGDRGKVVRYYAVAGLNTGARVFDVFTPAQIKTLRGGKVGSNGDIPDPEHWMERKTALLQVLKLMPKSTQLAAVPAADGRAHTISDAQQIFGGVDTSTGEVLEAEPVEGDAA

SEQ ID NO: 238 - protein, 265 aa, unidentified
ETIDIKQELASQAQTDSKKEVKLTKAMSIAEMIKAMMPEIKRALPSMITPERFTRIALSALNNTPELQACTPMSFISALLNAAQLGLEINSPLGHAYLIPYKNKGVLECQFQIGYLGLIALAYRNELMQTIQAQCVYENDEFLYEYGLNPKLVHRPATSDRGEPVFFYGLFKMINSGFGFCVMSKQEMDEFARTYSKGLASSFSPWKTSYNEMAKKTVIKQALKYAPIKTDFQKALSTDESIKYAISEDMTEAVNEIVSQNTEVA

SEQ ID NO: 239 - protein, 272 aa, unidentified
SNLKNQLANKAGGTATKKQPQTMQDWIKVMEPQIKKALPSVITAERFTRMALTAISTNPKLAECTPESFMGALMNAAQLGLEPNTPLGQAYLIPYGKSVQFQVGYKGLMELAQRSGQFKSIYAHTVYENDEFEVEYGLTQNIVHKPNFDDRGKPIGFYAVYKLTNGGENFVFMTQREVEEFGKAKSKTFNNGPWKTDFEAMAKKTVLKQLLKYAPIKVEFQREIAQDATIKTEIAEDMTEVPEEMVEAEYEVVEQNTMAEDADLKGTPFETK

SEQ ID NO: 240 - protein, 244 aa, unidentified
SNNELLAKPVEFEVNGEAVKLTGKTVKNFLVSGNGEVSDQEVVMFINLCKYQKLNPFLNEAYLVKFKSKSGPDKPAQVIVSKEAFMKRAEKHPNYEGFEAGIIVERDGQLVDIEGAIKLTNDKLVGGWARVYRSDRQKPITTRISLSEFSKGQSTWNSMPLTMIRKSAIVNAQREAFPETLGALYTEDDAKLDTTSSHDQEQVIEQEIKTKANQEVIDVEYTEESEQKSPQQEQTETTQAGPGF

SEQ ID NO: 241 - protein, 270 aa, unidentified
ATNSSLKNQLSKKENVTIGNTMQGLLNNPKMKKRFEEILDKKAPQYMSSILNLYNGDTSLQKCEPMSVLSSSMIAATMDLPVDKNLGYAWIVPYKNKAQFQMGYKGYIQLALRTGQYKHINAIEIHEGELVNWNPLTEELEIDFTKKESDKIIGYAGYFELLNGFKKSTYWTKTQIENHRKKFSKSDYGWNKDFDAMAIKTVIRNMLSKWGILSIEMQNAYTADENIIKDSFIDDSENVSANIEDLVEADYTVNQDSLESKEEFEGTPLE

SEQ ID NO: 242 - protein, 163 aa, unidentified
KKMTVMKTSAPLCYADVAEVKCEEFYEDQYKAGAEELFDNTSYDRLKVYLEKHGGLEGVHADVVRAGDTFVYRPGVIRRHGYVPGEQRGQVYAVYAKAHIKGGATRCVILARHEVEIDMDAKHGGNPDGDWENLAKVVALRSLAEALPLPSAVLQSCRTWSAK

SEQ ID NO: 243 - protein, 266 aa, unidentified
SNPPLAQADLQKTQGTEVKTKTKDQQLIHFINQPSMKAQLAAALPRHMTPDRMIRIVTTEIRKTPALANCDMQSFIGAVVQCSQLGLEPGNALGHAYLLPFGNGKAKSGQSNVQLIIGYRGMIDLARRSNQIISISARTVRQGDSFHFEYGLNENLTHVPGENEDSPITHVYAVARLKDGGVQFEVMTHNQIEKVRASSKAGQNGPWVSHWEEMAKKTVIRRLFKYLPVSIEMQKAVILDEKAEANVDQENASVFEGEFEEVSQSA

SEQ ID NO: 244 - protein, 244 aa, unidentified
ANELGIFSVDNLNMTTIKQYLDGGGKASDAELVLLINLCKQNNMNPFMKEVYFIKYGNQPAQIVVSRDFYRKRAFQNPNFAGIEVGVIVLNKDGVLEHNEGTFKTHEQELVGAWARVHLKNTEIPVYVAVSYDEYVQMKDGHPNKMWTNKPCTMLGKVAESQALRMAFPAEFSGTYGEEELPEPEKEPREVNGVKEPDRAQIESFDKEDYAAKKIEELKEKAQPQKEVVKETGEVIDKITAEDF

SEQ ID NO: 245 - protein, 244 aa, unidentified
ANELGIFSVDNLNMTTIKQYLDGGGKASDAELVLLINLCKQNNMNPFMKEVYFIKYGNQPAQIVVSRDFYRKRAFQNPNFAGIEVGVIVLNKDGVLEHNEGTFKTHEQELVGAWARVHLKNTEIPVYVAVSYDEYVQMKDGHPNKMWTNKPCTMLGKVAESQALRMAFPAEFSGTYGEEELPEPEKEPREVNGVKEPDRAQIESFDKEDYAARKIEELKEKAQPQKEVVKETGEVIDEITAEDF

SEQ ID NO: 246 - protein, 269 aa, unidentified
TNNQLVEAKGDFLTNPQLLNSGIIRKYLDPQGKASDEELAYFIAQAKAQNLNPFTKEIYFIKYGTQPAQIVTAKSAFEKKADSHPQFDGKEAGVIYLLDGEIKYSKGAFIPKGAEILGGWAKVYRKDRTYPTETEVSFEEYDNSKIRARVKELTQQGKDVTYPVMNSYGKPIGENNWDTMPCVMIRKVALVSAYREAFPAELGASYEADEIQLDNTPKDITPQENREDVIARKMAQIEQFNKEQAHTDPEPTQTEEPIQGELLDGELEY

SEQ ID NO: 247 - protein, 268 aa, unidentified
RTDGTKEAGAAATAPTEGKAPAKAHKPADTIGAMIEKLKPQIERALPKHVTPDRMARMALTAIRNNPKLGQAEAVSLMGSIIQASQLGLEPNTPLGQCYIIPYNSKNGMQAQFQMGYKGIVDLAHRSGQYRQLTAHPVDEADEFRYSYGLNPDLVHVPAEKPSGKITHYYAVYHLTNGGFDFRVWSREKVEAHAKQYSKSFSSGPWQTNFDQMACKTVMIDLLRYAPKSVEIAKATSADNRTHTINPEDPDLNIDTIDGDFELEGEER

SEQ ID NO: 248 - protein, 261 aa, unidentified
EALLLRRWQMGNLTKTTGFALAPQNLEQAMQLATMICNSQLAPNNYKGKPEDTLVAMMMGHELGLNPLQSIQNIAVINGRPSIYGDALLALVQNSPAFGGIQESFDEDTMTATCTVWRKGGEKHTQHYSKDDADTAGLWGKQGPWKQHPKRMLAMRARGFAVRNQFADALAGLVTREEAEDMEKEINPTPAPQAQSKRIGQKQSRTQYSESDFNENFPKWKAAVESGKKTSEQIISMVSTKGDLTQGMIEAIESIEAGEPA

SEQ ID NO: 249 - protein, 241 aa, unidentified
GHLVSKTEQDYIKQHYAKGATDQEFEHFIGVCRARGLNPAANQIYFVKYRSKDGPAKPAFILSIDSLRLIAHRTGDYAGCSEPIFTDGGKACTVTVRRNLKSGETGNFSGMAFYDEQVQQKNGRPTSFWQSKPRTMLEKCAEAKALRKAFPQDLGQFYIREEMPPQYDEPIQVHKPKALEEPRFSKSDLSRRKGLNRKLSALGVDPSRFDEVATFLDGTPDRELGQKLKLWLKEAGYGVNQ

SEQ ID NO: 250 - protein, 257 aa, unidentified
TDVKQELERKVGKQDSTAVRLTKNMSIPDMIKALEPEIRRALPAVLTPERFLRMALSAVNNTPKLAECTPMSFIAAMMNAAQLGLEPNTPLGQAYLIPYKNKNQLECQFQIGYKGIIDLAYRTGQMQMIQAHAVHEFDDFEYEYGLNPKLIHRPGDGNRGEITYFYGLFKLVNGGFGFEVMNREAMEAFAQQYSQSYGSQYSPWVKNFEDMAKKTVIKKALKYGPVKAEFQKAISMDETIKTEIAVDMTEVQNEESE

SEQ ID NO: 251 - protein, 208 aa, unidentified
ENEVMTQDQAYEVASPFGSSENFQKLFDIGKMFASSSLVPDRYRGKPMDCTIAVDMANRMGVSPMMVMQQLYVVKGNPQWSGQACMSLIRGSSEYKNVRPVYTGKKGEDSWGCYIEAEKKKTGEIVKGTEVTIAMAKAEGWYSKKDKYGNETSKWQTMPELMLAYRAAAFFARVYIPNALMGCAVEGEAEDIMKRAITAEDPFKEDAK

SEQ ID NO: 252 - protein, 258 aa, unidentified
TLQAVCPTQDKAVESQLDQTKFELIKRTICKGTTDDEFQLFIHACKRTGLDPFMRQIFAVKRWDSAERREVMTIQTGIDGYRLIADRTGRYAPGRDAEFGYDAHGGLRWAKAYVKKMTPDGHWHEISATAFWTEYVQTTKDGRPTVFWMKKGHVMLSKCAEALALRKTFPAELSGIYTQEEMAQTMSLPDTKGDSQTIGSDKAYEIERSIDNDPEFKTQLLTRLQRAFGCKSFSDLPQDQFKNVKKVIENHQIKEKIA

SEQ ID NO: 253 - protein, 214 aa, unidentified
  source 1..214: note = Fimbriiglobus ruber
TDIAHRSYSAPQLSLIRRTVAKDTNQDEFDLFIEICKQQGLDPFKKQIFAQVYNKDKADKRQIVIVTSIDGYRAKAQRCGDYRPAEEETRFEADAALKIRRQPMGSFVRSCGLQVRPGQGVVSRVGEARWDEFAPLDDAEFDWVDTGETWPDTGKPKKKKVAKSAKKTLKEGNWKNMPHVMLGKCAEAQALGGGGRKRSAACTSKRRWTRSTWT

SEQ ID NO: 254 - protein, 235 aa, unidentified
TASKQTDIFSFVSGGEDITITLADIKNYFCANATDQECVLFGQLCKANGLNPWLKEAYLIKYDKNAPAAMVTGKDAYMKRANEHPAFDGYEAGVKVYLPDVGQVEYREGTAYYEDLGEQLIGGYAKVYRKDRSRPYYEEVPLKEYDTKQSKWKTSPATMIRKVALVHALREAFPTNIQGMYDADETPYAADYEGSFREMDDPTPAPSMRGRIAPAPVADPLEDLEADVIEAGDVE

SEQ ID NO: 255 - protein, 265 aa, unidentified
ADLTKTANGADLAAAIGGKQAETGRATAFDLVKSMEAEFAKALPRHVPVEQFMRTAVTELRQNADLQRSTSESLLGAFLTAARLGLEVGGPMGEFYLTPRFAKLPGQDQKAWQVVPIVGYRGLVKLARNAGVGAVKAWVVYEGDHFVEGANSERGPFFDFHPVPGDPAGRKEVGVLAVARLSGGDVQHTYLTIEQVEKRKARGSAGDKGPWATDRAAMIRKSGIRALAGELPQSTLLALARVVDEEVQTYVPGSLVDVGTGELEA

SEQ ID NO: 256 - protein, 247 aa, unidentified
NTELETMNNVYDNLQSVIMQQGIAALLPAQVTPEQFTRTAATALIENVDLQNADKQSLVLALTRCAKDGLMPDGREAALVVRSTKVNKQFVKKAVYMPMVDGVIKRARQSGQVANIIAKVVYSQDEFEYVIDENGEHLTHRPAFVDGDDIVKVYAFAKLNSGELVVEVMSRAGVEKIRDTVQSAKYDSSPWVKWFDRMALKTVIHRLARRLPCASELFSLFEVYEDANSTEKTLRMAPASFKRLSIN

SEQ ID NO: 257 - protein, 269 aa, Raoultella planticola
TKQPPIAKADLQKTQGTRVSSPKGNNDVISFINQPSMKEQLAAALPRHMTAERMIRIATTEIRKVPALASCDTMSFVSAIVQCSQLGLEPGGALGHAYLLPFGNKNEKSGKKNVQLIIGYRGMIDLARRSGQIASLSARVVREGDEFSYEFGLEEKLTHRPGENEDAPVTHVYAVARLKDGGTQFEVLTSKQIELVRSQSKAANSGPWVTHWEEMAKKTAIRRLFKYLPVSIEIQRAVSIDEKEALTIDPADTSVLTGEYSVINSESEE

SEQ ID NO: 258 - protein, 272 aa, Pantoea brenneri
SNQPPIASADLQKTQQSKQVANKTPEQTLVGFMNQPAMKSQLAAALPRHMTADRMIRIVTTEIRKTPQLAQCDQSSFIGAVVQCSQLGLEPGSALGHAYLLPFGNGRSKSGQSNVQLIIGYRGMIDLARRSGQIVSLSARVVRADDEFSFEYGLDENLVHRPGENEDAPITHVYAVARLKDGGTQFEVMTVKQVEKVKAQSKASSNGPWVTHWEEMAKKTVIRRLFKYLPVSIEMQKAVVLDEKAESDVDQDNASVLSAEYSVLESGDEATN

SEQ ID NO: 259 - protein, 267 aa, Xenorhabdus poinarii
TNTPPLAQADLQKAQPQTKVAATKDQALIQFINKPSMKAQLAAALPRHMAPDRMIRIVTTEIRKTPALANCDMQSFVGAVVQCSQLGLEPGSALGHAYLLPFGNGKSKTGQSNVQLIIGYRGMIDLARRSGQIVSISARTVRDGDQFHYEYGLNENLTHIPGENEDAPITHVYAVARLQDGGVQFEVMTRKQVEKVREKSSAGNNGPWVTHWEEMAKKTVIRRLFKYLPVSIEMQKAVILDEKADANIDQDNAAIFEGEFEEVGNDG

SEQ ID NO: 260 - protein, 267 aa, Proteus mirabilis
SNPPLAQADLQKTQGTEVKTKTKDQQLIHFINQPSMKTQLAAALPRHMTPDRMIRIVTTEIRKTPALANCDMQSFIGAVVQCSQLGLEPGNALGHAYLLPFGNGKAKSGQSNVQLIIGYRGMIDLARRSNQIISISARTVRQGDSFHFEYGLNENLTHVPGENEDSPITHVYAVARLKDGGVQFEVMTHNQIEKVRASSKAGQNGPWVSHWEEMAKKTVIRRLFKYLPVSIEMQKAVILDEKAEANVDQENASVFEGEFEEVGSNGN

SEQ ID NO: 261 - protein, 266 aa, Edwardsiella piscicida
TNNQQPPIATADLQKAQSQAPAVKPDQKLINFINQPSMKGQIAAALPRHMAPDRMIRIITTEIRKTPALATCDMQSFIGSVVQCSQLGLEPGGALGHAYLLPFGNGKAKSGQSNVQLIIGYRGMIDLARRSGQIVSISARTVRDGDQFHYEYGLDETLKHVPGDNESSPITHVYAVAKLKDGGVQFEVMTFNQIEKVRGQSKAGNNGPWQTHWEEMAKKTVIRRLFKYLPVSIEMQKAVILDEKAEANIDQENASVISAEFSVVED

SEQ ID NO: 262 - protein, 265 aa, Morganella morganii
SNPPIAQADLQKAQGTAVKEKTKDQQLIQFINQPGMKAQLAAALPRHITPDRMIRIVTTEIRKTPSLATCDMQSFIGAVVQCSQLGLEPGNALGHAYLLPFGNGKAASGQSNVQLIIGYRGMIDLARRSGQIISISARTVREGDSFHFEYGLNEDLTHVPGENDSGPITHVYAVARLKEGGVQFEVMSFSQIEKVRDSSKAGKNGPWVSHWEEMAKKTVIRRLFKYLPVSIEMQRAVILDEKAEANVDQEHASIFEGEYETVSPE

SEQ ID NO: 263 - protein, 267 aa, Providencia alcalifaciens
STPPLAKSDLQKTQGTEVKIKTNEQKLVEFINQPGMKAQLAAALPKHITSDRMIRIVSTEIRKTPSLANCDIQSFIGAVVQCSQLGLEPGNALGHAYLLPFGNGKSDNGQQNVQLIIGYRGMIDLARRSGQIISISARTVRQGDNFHFEYGLNENLTHIPEGNEDSPITHVYAVARLKDGGVQFEVMTYNQIEKVRNLSKAGKNGPWVTHWEEMAKKTVIRRLFKYLPVSIEMQKAVILDEKAEANIEQEHSAIFEAEFEEVDSNGN

SEQ ID NO: 264 - protein, 271 aa, Proteus terrae
SNPPLAQADLQKTQGTEVREKTKDQMLVEFINKPNMKAQLAAALPRHMAPDRMIRIVTTEIRKTPELANCDMQSFVGAVVQCSQLGLEPGNALGHAYILPFEKKRKQGNQWVTVRTDAQLIIGYRGMIDLARRSGQIVSISARTVRQGDSFHFEYGLNENLTHVPGENEDSPITHVYAVARLKDGGVQFEVMTHNQIEKVRTSSKAGQNGPWVSHWEEMAKKTVIRRLFKYLPVSIEMQKAVILDEKAEANVDQENSSVFEGEFEEVGQGA

SEQ ID NO: 265 - protein, 267 aa, Plesiomonas shigelloides
SNQRPPIATADLQKAQSQPPAAKPEQNLINFINQPSMKSQIAAALPRHMAPERMIRIITTEIRKTPKLATCDVQSFIGAVVQCSQLGLEPGGGLGHAYLLPFGNGKAESGKPNVQLIIGYRGMIDLARRSGQIVSISSRIVREGDQFHYEYGLNETLKHVPGDNESAPITHVYAVAKLKDGGTQFEVMSFNEIEKIRGQSKAGNDGPWIKHWEEMAKKTVIRRLFKYLPVSIEMQRAVILDEKAEADIEQDNASIIGAEYSVVENAA

SEQ ID NO: 266 - protein, 265 aa, Gilliamella apicola
SEQNQPPIAKSDLEKTQLTNQDKKPATLAELVNSPKIKNQLAMALPKHMNPDRMARIVTTEIRKTPALADSNIQSFLGAVVQCSQLGLEPGGALGHAYLLPFGNGKAKDGKSNVQLIIGYRGMIDLARRSGQIISISARTVREGDDFHYEYGLNEDLKHTPKADESAPITYVYAVARLKDGGSQFEVMTFNQIESVRKQSKAGDKGPWITHWEEMAKKTVIRRLFKYLPVSIEIQQAVILDEKAEAGISQDNEMILDADFSVVEA

SEQ ID NO: 267 - protein, 263 aa, Pseudomonas fluorescens
NSTAETATPFSSQDLEKTQPTKAQSKTGSLASLLASPKMKSQFAAALPKHMTPERMARIVTTEIRKNPELVKCEQHSFLGAVIQCAQLGLEPGNTLGHAYILPYGKQAQLIIGYRGMIDLARRSGQIISISARTVREGDYFEYEFGLDENLIHRPVETTQPGAVTHVYAVARLKDGGRQFEVMSRAQIEEVRVQSKAAKSGPWVTHWEEMAKKTVIRRVFKYLPVSVEIQRAVMLDEKAEAGVCQENECVFDGDFEVITDTEE

SEQ ID NO: 268 - protein, 266 aa, Pseudomonas stutzeri
STENVAPFSQKDMQQATGQQVKPRSPADSLAAMLASPKMKAQFAAALPKHMTAERMARIVTTEIRKTPALVKCDQHSFLGSVIQCAQLGLEPGNSLGHAYLLPYGNQVQLIIGYRGMIDLARRSGQIVSLSARTVREHDEFDYQLGLHEDLTHKPFEGEHAGEITHVYAVARLQGGGVQFEVMSKAQVEAVRAQSKAGKSGPWVSHWEEMAKKTVIRRLFKYLPVSVEIQRAVTLDEAAEAGLPQGNEYVFDGDFEVVNDASGAQQ

SEQ ID NO: 269 - protein, 258 aa, Methylothermus sp.; identical to SEQ ID NO: 230

SEQ ID NO: 270 - protein, 256 aa, unidentified
  source 1..256: note = Halomonadaceae bacterium R4HLG17
SEVATQDTLGKELQQHSGQQKKPMPTTIQGMLKDPRFTSQIARALPKHITPDRITRIALTEVNKTPALGKCDPVTLFGSIIQSAQLGLELGGALGHAYLVPYGNQAQFIIGYRGMIDLARRSGQMVSLQAHTVHDNDEFDFEYGLDEKLRHVPARGDRGPMVAVYSVAKLVGGGHQIEVMWKEDVDAIRSKSKAGNSGPWRDHYEEMAKKTAIRRLFKYLPVSVEMQKAVALDEQAEAGVQDNNVFDGEFSYGEAE

SEQ ID NO: 271 - protein, 257 aa, Halomonas coralii
TDQATAEPQEDLGKQLQQHSQRKPMPTTIQGMLKDDRFTGQIARALPKHITPDRISRIALTEVNKTPALGKCDPMSLFGSIIQSAQLGLELGGALGHAYLVPYKDQAQFIIGYRGMIDLARRSGQMVSLQAHTVHENDDFEFEYGLDEKLRHVPARGQRGPMIAVYAVAKLTGGGHQIEVMWKEDVDAIRQQSKAGNSGPWRDHYEEMAKKTAIRRLFKYLPVSVEMQKAVSLDEQAEAGVQDNNVFDGEFSYQEPE

SEQ ID NO: 272 - protein, 262 aa, Zymobacter palmae
TNTVQQQAPQQDQLAQQLQQASGNTPQKKPMPSTIQGMLKDDRFKTQIARALPKHVTPERIMRIALTEINKTPKLKECDPIGLFGSIVQSAQLGLELGGALGHAYLVPYGKQAQFIIGYRGMIDLARRSGQMVSLQAHTVHENDEFNFEYGLNENLRHVPARGERGPMIAVYAVAKLVGGGHQIEVMWKEDVDAVRKSSKAGGSGPWRDHYEEMAKKTAIRRLFKYLPVSVEMQRAVSLDEQAEEGVQDNNVFDGDYTVAEH

SEQ ID NO: 273 - protein, 271 aa, Salinicoccus albus
STNESLKNQVATNQKNEVSNGNKPKTIGDYIDQMAPAMAQALPKHMSVERMTRMATTVIRTTPQLKEADVASLLGAVMQSAQLGLEPGPMGHCYFLPFKNNKKGTTEVTFIIGYKGMIDLARRSGHISTIYAHAVYENDEFEYELGLHADLKHKPSEDERGAFKGAYAVAHFKDGGYQFEYMPKSDIDKRRSRSKAGNSNYSPWATDYEEMAKKTVIRHMWKYLPVSVEMQQAVAHDEGTGKDIKDVTPDEDSFVDMPEYIADVPAEGEGE

SEQ ID NO: 274 - protein, 266 aa, Lysinibacillus sp.
ATTTDLKAQMQQAPATQQKPKTIDDYLKQMAPAMAQALPKHMDVDRLMRLAMTTIRTTPALKDADVSSLLGAVMQAAQLGLEPGLMGHCYLLPFKNNKKGITEVQFIIGYKGMIDLARRSGHIQSIYAHAVYQKDEFEYELGLDPKLKHKPCMDEDKGNFVGAYAVAHFKDGGYQFEFMSKAEIEKRKGRSKAANSTYSPWATDYEEMAKKTVVRHMWKYLPISVEMQQQVAYDEGTAPKREMKDITPETEFFVDAPEIEVEVVNE

SEQ ID NO: 275 - protein, 272 aa, Tepidibacillus fermentans
ATNEKVKTQLANRANGQAPTPTPEQTIAAYMKKMAPRFAEVLPKHMDIDRMTRIALTTIRTNPKLLEASVPSLLGAIMQAAQLGLEPGLVGHCYLVPFKNGKTGQTEVQFIIGYKGMIDLARRSGNIESIYAHAVYENDTFEYEYGLHPKLVHKPAMTDRGEFIGAYAVAHFKDGGYQFEFMPKEEIEKRRNRSKTANGGPWVTDYEEMAKKTVVRHMWKYLPISIEIQQAAAQDEVIRKDVTSEPEFVDDVIDISTEIEEQSVEVEGEEAQ

SEQ ID NO: 276 - protein, 271 aa, Ammoniphilus oxalaticus
STKATSNELKNQLANRQGNNAATNNNPANTIAAYLKRMAPEIEKALPAHMDADRLARIALTTIRTTPKLLECTIPSLMGAVMQSAQLGLEPGLIGHCYIIPYGKEATFIIGYKGMIDLARRSGNIESIYAHAVYKNDEFEYEYGLKPNLVHKPAMSDQGDFIGAYAVAHFKDGGYQFEFMPKEEIDKRRNRSAASKGGPWVTDYEEMAKKTVVRHMWKYLPISIEIQQAATQDEVVRKDITEDPMPVDVLDIPFEASDAEETSEEGEINFD

SEQ ID NO: 277 - protein, 267 aa, Rummeliibacillus stabekisii
ATTTELKEQMKQQAPAQTKKPKTIEDYMKQMAPAMAEALPKHMSVDRLTRLAMTTIRTTPALRQADVSSLLGAVMQAAQLGLEPGLLGQCYLLPFKNKKKAITEVQFIIGYKGMIDLARRSGHIQSIYSHAVFENDVFEYELGLEPKLKHTPTMSTDKGAFIGAYAVAHFKDGGHQFEFMSKADIEKRKGRSKAANSDYSPWLTDYEEMAKKTVIRHMWKYLPISVEMQEQVAYDEGVGRSIKDVTPEEDVFVQAPDEILEAEATEA

SEQ ID NO: 278 - protein, 272 aa, Bacillus sp.
TQAEKLKNDIAKQEQKNEVAQDDKPKTILDVMMQHKESFEMALPKHLDADRLIRLAVTEFRKNPMLKECTPESLLGAVMQAAQVGLEPDALGSAYLVPYYNKNKNVKEVQLQIGYKGLIELVRRSGQVTSIVANEVYENDEFDFEYGINEKLYHKPTMDADRGKLKCFYAYARFKDGGHAFTVMSVEQINQIRDKFSKSQKNGKHFGPWADHYESMAKKTVIKQLVKYMPISVEIQNQITRDETVHSSFKEEPKPIYAFEESPDIIDAPIEN

SEQ ID NO: 279 - protein, 272 aa, Aneurinibacillus tyrosinisolvens
SDLKEKLEKRANETEAAPPSPAQTIAAYLKRMEPEIARALPKHMDVERLTRIALTTIRTNPRLLECTVPSLLGAVMQAAQLGLEPGLLGQCYIIPHGREATFIIGYKGMIDLARRSGNIKSIYAHDVRENDEFEYEYGLHPFLKHRPAMTDRGKFIGVYAVAHFNDGGYQFEFMPYEEIERRKLRSRSYKNGPWVTDYEEMAKKTVIRHMFKYLPLSVEIMRSAAQDETVRPDLTSDPVSIYERPIEGKIITAEDVQPEEIPNVPDAEQGDV

SEQ ID NO: 280 - protein, 268 aa, Siminovitchia fordii
ATNQDIKNQLANKANGNKPASPANTIAAYLKKMGPEIEKALPKHMDADRLARIALTTIRTTPKLLECNISSLMGAVMQSAQLGLEPGLIGHCYIIPYGKEATFIIGYKGMIDLARRSGQIQNIYAHAVFENDEFDYALGLHPKLEHKPAGSNRGEIIGAYAVAHFKDGGYQFEYMAKEDIEKRKSRSAAARSKHSPWATDYEEMAKKTVIRHMWKYLPISVEIQQQAIQDEVVRKDVTSEPEFIDMEDMPEVEEGQSEESEQVEAPFD

SEQ ID NO: 281 - protein, 266 aa, Bacillus sp.
ATNKDVKNQLANRKENKPATPEQKVEAYMTAMAPRFAEVLPKHMSMDRMSRIALTTIRTNPKLLECSVPSLMGAVMQAVQLGLEPGLLGHCYILPYKSEATFIIGYKGMIDLARRSGHIQSIYAHAVYENDEFDYELGLHPKLTHKPSFGERGEFIGAYAVAHFKDGGHQMEFMPKSEIEKRRSRSASGNSSYSPWKSDYEEMAKKTVVRYMFKYLPISIEVQSQAQHDEVVRKDITEEPEFIEMDSIEVAEASEGDGQKEFVIEE

SEQ ID NO: 282 - protein, 269 aa, Acinetobacter sp.
ADLKNKLANKAAGTVTKTSPNAGMKQLMKSMSKEIEAALPSHMSSERFQRVALTAFGNNPKLMNCDPMSFIAAMMDSAQLGLEPNTPLGQAYLIPYGTKVQFQVGYKGLLELALRSGKIKTLYAHEVRENDTFEVKYGLHQDLIHEPVLKGNRGEVIGYYAVYHLDTGGHSFVFMTKDEVLEHAKGKSKTFNNGPWQTDFDAMAKKTVIKQLLKYAPLSIEMQKAVSSDETVKSKIDEDMSLVVDESDSIEANFEIKEDEDGQLDVYVK

SEQ ID NO: 283 - protein, 252 aa, Methylomonas rhizoryzae
SELLSALNAPETQKPQTLPAMLKQHQPRFKAIAPRDVDVTRFSAALMADVRSNQKLAECNPMTVLGAFIRSTQLGLEPGSQLGQAYFVPFKGECQLVIGYRGMIELAYRSGKVASISARTVYENDVFEWELGTDERITHKPATGDRGALVAVYAMAKLTTGGIHFEVLDLAEIEKAKRASKSSSFGPWKDHFEEMAKKTAIRRLFKYLPVGTDLTRAVALDEKAESGSQQNDIEAETVLDGEFYPAGGGNDG

SEQ ID NO: 284 - protein, 265 aa, Lacrimispora amygdalina
AVDVKNELERKASGQNSQVKLTKSMTIADMVKALEPEIKRALPAVLTPERFTRMALSAINNTPELAGCTPMSFIAAMMNAAQLGLEPNTPLGQAYLIPYKNKGVLECQFQIGYKGMIDLAYRTGQIQVIQGQAVREFDYFEYQYGLDPKLVHRPGEEERGEITFIYGLFRLSNGGYGFEVSNKADMDAFAAKYSKSFGSKYSPWTENYEDMAKKTVIKRALKYAPVSVDFQKAMSMDETIKTEISVDMSEIRNECPEISENGEAA

SEQ ID NO: 285 - protein, 257 aa, Lachnoclostridium sp.; identical to SEQ ID NO: 250

SEQ ID NO: 286 - protein, 265 aa, Enterocloster bolteae
GVNVKHELEQRAAGQGASVRLTKNMTIVDMVKALEPEIRRALPAVLTPERFTRMALSSINNTPELAECTPMSFIAALLNAAQLGLEPNTPLGQAYLIPYKNKGKLECQFQLGYKGLIDLAYRTGQVQIIQAQVVREFDSFEYQYGLDSKLVHKPGEGARGEITYVYGLFKLSNGGYGFEVSNKTEMDTFAARYSKSFGSKYSPWTEDYESMAKKTVIKRVLKYAPISSDFQKALSMDETIKTGIAVDMSEIRNECLPEEAGSEAA

SEQ ID NO: 287 - protein, 264 aa, Kribbella albertanoniae
ATADSVREELARSKEVERTQPKASNADNVIGLINRSLPEIAKALPGHVKPERIARIATTAVRVTPKLADCTQASFLGALLTAAQLGLEPNTPTGEAYLLPFGRNVQLIIGYRGYIKLANQSGQVRNIMAMTVYENDHFDYKYGSNPFLEHTPTLGQDPGPVKCWYACATFTNGGTNFVVLDKFKVEGYRARARSKDDGPWVTDYDAMARKTCIRRLAPYLPMSVELAQAMQVDEEVTAFTPGVSDPEVLATLAGVDTGTGEVQQ

SEQ ID NO: 288 - protein, 267 aa, Bacillus albus
ATNEKLKNQLANRKESAPATPEQTVEAYMKKMAPKMAEVLPKHMDMGRMSRMALTTMRTSPKLLNCTVSSLMGAVMQAVQLGLEPGLLGHCYILPYKGEATFIIGYKGMIDLARRSGHIQSIYAHAVHENDEFDYELGLHPKLEHKPVHGDRGAFVGAYAVAHFKDGGYQMEFMPKSEIEKRRKRSASANSSFSPWKSDYEEMAKKTVIRYIFKYLPISIEVQLLAAQDEVVRKDITEEPEFIEADPIDVEQPTTEGDGQQEFSIEE

SEQ ID NO: 289 - protein, 267 aa, Bacillus cytotoxicus
ATNEKIKNQLANRKANASLSPEQTVEAYMKKMAPRFAEVLPKHMDMDRMSRIALTTIRTNPKLLECNVPSLMGAVMQAVQLGLEPGLLGHCYILPYKGEATFIIGYKGMIDLARRSGHIQSIYAHAVYENDEFEYELGLNPQLKHKPSFGDRGEFIGAYAVAHFKDGGHQMEFMPKSEIEKRRKRSASANSNYSPWKSDYEEMAKKTVVRYMFKYLPISIEVQSQAQHDEVVRKDITEEPQFIEADSVEVEETPTEGTNQEEFVIEE

SEQ ID NO: 290 - protein, 262 aa, Bacillus sp.
ATNKDVKNQLANRKASAPVTTEQTVEAYMKKMGPKMAEVLPKHMDMDRMSRIALTTIRTNPKLLECSVPSLMGAVMSAVQLGLEPGLLGHCYILPYKSEATFIIGYKGMIDLARRSGHIQSIYAHAVYENDEFEYELGLHPQLKHKPSFGDRGEFIGAYAVAHFKDGGHQMEFMPKSEIEKRRGRSASANSNYSPWKTDYEEMAKKTVVRYMFKYLPISIEVQSQAQQDEVVRKDITEEPEFIEVEQQTEGDGQGDFVIEGE

SEQ ID NO: 291 - protein, 265 aa, Clostridium transplantifaecale
TDVKEELARKAGNTGKQEIRLNKNMSIPDMVKVLEPEIKRALPSVLTPERFTRMALSAINNTPKLAECSPMSFIAALMNAAQLGLEPNTPLGQAYLIPYKNKNQLECQFQIGYKGYIDLAYRTGQVQMIQAQAVHEFDYFEYEYGLTPKLVHRPGEGERGEITYFYGLFKMINGGFGFEVMNRAAMDAFAKQYSQSINSKYSPWNSQYEEMAKKTIIKKALKYGPVKSDFQKAISMDESIKTELSIDMSEVRNEDLIDGEFEEAA

SEQ ID NO: 292 - protein, 265 aa, Lachnoclostridium pacaense
TDVKQELEKRAGSSNQAIKLTKSMTIVDMVKALEPEIKRALPAVLTPERFTRMALSAINSTPKLAECTPMSFIAALMNAAQLGLEPNTPLGQAYLLPYKNKGVLECQFQIGYKGVIDLAYRTGQIQMIQAQAVRESDYFEYQYGLEPKLVHRPGDGARGEVTFIYGMFRLTNGGYGFEVSNKADMDAFAEKYSKSYGSRYSPWTENYEDMAKKTVIKRALKYAPISSDLQKALSSDETIKTVLSVDMSEINNECQIDEVIQEDAA

SEQ ID NO: 293 - protein, 267 aa, Clostridium sp.
SVDVKNELEKRAAGTVNPAVKLTKNMTIVDMVRALEPEIKRALPTILTPERFMRMALSAINNTPELADCTPMSFIAALMNAAQLGMEPNTPLGQAYLIPYKNKGTLECQFQIGYKGLIDLAYRTGLIQVIQAQTVREFDSFEYQYGLDSRLTHRPGDGERGEITYIYGLFKLTNGGYGFEVSNKADMDAFAEKYSKSFGSRFSPWKENYEDMAKKTVIKRALKYAPVSSDFQKALSMDETIKSELSIDMSEIRNECQVEASGQEGAA

SEQ ID NO: 294 - protein, 267 aa, Lysinibacillus fusiformis
ATTNELKAKSQNQVQQNVTPEQSLNTLLKRMGPQIQRALPKHMDADRIARIALTAVRATPKLLECDQMSFVAALMQSAQLGVEPNTGLGQAYLIPYGKQVQFQLGYKGLIDLAVRSGQYKAIYAHEVYKEDEFSFAYGLHKDLVHVPSTNPEGEPIGYYAVYHLKNGGYDFVYWTRERIDKHAHEFSQAVKKGWTSPWKTNYDAMAKKTVLKEVLKYAPKSIELQKVVEADETIKTEVSEDMSDVIDVTDYSVIEDESAQEELIIEQ

SEQ ID NO: 295 - protein, 268 aa, Marispirochaeta aestuarii; identical to SEQ ID NO: 247

SEQ ID NO: 296 - protein, 265 aa, Solibacillus sp.
ATSNELKKQAQGQVTAKPTTPEGSLNALLKKMGPEIQRALPKHMDADRIARIALTAVRTTPKLLECDQLSFVAALMQSAQLGVEPNTGLGQAYLIPYGGKVQFQLGYKGLIDLAVRSGQYKAIYAHEVYADDEFSFAYGLHKDLVHVPSANPSGDPIGYYAVYHLKNGGYDFVYWTRERIDIHSKAFSQAVQKGWTSPWKTNYDAMAKKTVLKEVLKYAPKSIEMQKVVEADETIKNEVAPDMSNVIDVTDYSILEDPQDVTDAQ

SEQ ID NO: 297 - protein, 266 aa, Viridibacillus sp.
ATNNALKEQMKQAPSKEVKPEQSLNTLLKRMGPEIQRALPKHMDADRIARIALTAVRNTPKLLDCDQMSFVAALMQSAQLGVEPNTGLGQAYLIPYGKQVQFQLGYKGLIDLAVRSGQYKAIYAHEVYEDDEFSFAYGLHKDLVHVPAPNPTGEPIGYYAVYHLQNGGYDFVYWTRERIDQHAHKFSMAVQKGWTSPWKTNFDAMAKKTVLKEVLKYAPKSIEMQKVVDADETVKTDVSDDMSNVIDVTDYTVMDQEQETIQEPTK

SEQ ID NO: 298 - protein, 268 aa, Lacrimispora indolis
SDVKQELEKRAAGGGGQSQSVRLTKNMTIVDMVKALEPEIKRALPSILTPERFTRMALSAINNTPKLGECTPMSFIAALMNAAQLGLEPNTPLGQAYLIPYKNKGVLECQFQLGYRGLIDLAYRNERMQSVEAQVVYENDEFSYELGLHPSLIHRPSFDEPGEIRAFYAIFRLDNGGFRFEVMSKSYVDAYAARYSKAFTSDFSPWKSNYEGMAKKTVIKQLLKYAPMKSEFQKAVTMDETIKTELSVDMSEVSNQEVIDRELTEQVA

SEQ ID NO: 299 - protein, 271 aa, Peptoanaerobacter stomatis
GAKELIQKKQENKQISPTSNMNMLLQSMAGAIKKALPAQINSERFQRVALTAFSSNQKLQQCDPISFLAAMMQSAQLGLEPNTPLGQAYLIPYGKQVQFQVGYKGLLELAQRSGQFKSIYSHEVRENDEFEMEYGLNQKLVHKPNLKQERGEVIGYYACYHLTNGGESMFFMTKDEIINFGKSKSKTFNNGPWQTDFDAMAKKTVLKQLLKYAPLSIESQKFMSMDETVKSDISANMDEINNDTVDFEVDIQTGEVINDIVVENTNEDEAN

SEQ ID NO: 300 - protein, 270 aa, Thermoclostridium stercorarium; identical to SEQ ID NO: 200

SEQ ID NO: 301 - protein, 265 aa, Enterocloster clostridioformis
GIDVKHELEKRAAGQDKPVKLTRNMTIADMVKALEPEIKRALPAILTPERFTRMALSAVNNTPELANCTPMSFIAALMNAAQLGLEPNTPLGQAYLIPYKNKGTLECQFQLGYKGLIDLAYRTGQIQIIQAQAVREFDYFEYQYGLDSRLVHKPGNEERGQITFIYGLFKLSNGGYGFEVSNKAEMDAFAAKYSKSFGSKYSPWTEDYESMAKKTVIKRALKYAPVSSDFQKALSLDETVKSEIAVDMSEIRNDCIPADMGTEAA

SEQ ID NO: 302 - protein, 268 aa, Lacrimispora celerecrescens
SDVKQELEKRAAGGGSQSQSVKLTKNMTIVDMVKALEPEIKRALPSILTPERFTRMALSAINNTPKLGECTPMSFIAALMNAAQLGLEPNTPLGQAYLIPYKNKGVLECQFQLGYRGLIDLAYRNDRMQSIEAQVVYENDEFSYELGLHPSLTHRPSFDEPGEIRAFYAIFRLDNGGFRFEVMSKSYVDAYATKYSKAFTSDFSPWKNNYEGMAKKTVIKQLLKYAPIKSDFQKAITLDETVKTQLSIDMSEIRNECLPDTSENSEVA

SEQ ID NO: 303 - protein, 272 aa, Andreesenia angusta; identical to SEQ ID NO: 239

SEQ ID NO: 304 - protein, 239 aa, Amycolatopsis arida
HGTALNPERFTRVALTVIRQSADLQRCRPESLLGALMTSAQLGLEPGPLGEAYLVPYGDQVTFIPGYRGLIKLAWQSGQLRHISARVVHEGDRFSYSYGLHPDLIHQPTRGDRGPITDVYAAATLIDGGVEFEVLDVATVETIRARSRAGRKGPWVTDWEAMARKTAIRQLAKWLPMATVMSRAIAAEGTVRTDLDADALDDLTADPGPEVLDADPAWDGPEPPGDQARNQEPTTQGDA

SEQ ID NO: 305 - protein, 271 aa, Corynebacterium striatum
GTNLEQRMAANNAPAKQNRPVTLADQIRSMESQFQLAMPKGMEAQQLVRDALTCLRQTPKLAECTPQSVLGGLMTCSQLGLRPGVLGHAYLLPFWDRKQGGMVAQLVVGYRGLVELAHRSGQIQSLIARTVYENDHFDVDYGLDDKLVHKPCMNGPKGNPIAYYAVAKFTTGGHSFIVMSKDEMLAYRDEFAKAKNKQGEVFGPWADNFDAMAHKTCVRQLAKWMPSSTDLDRGIAADETVRVDLSESALDYPQHVDGEVVDSKPAEDEAA

SEQ ID NO: 306 - protein, 261 aa, Gottfriedia acidiceleris
ATPAELKNLLAAKPKGEVKLTPDQQVSSYLKAYEGTFRQIAPKHFNTERFQRIALSEIRKNPKLLDCNLPSLMSAVLQSVKLGLEPGLFGQAYLIPYGKEVQFQIGYKGLIELAQRSGRIAKIQAREVYEHDEFEVSYGIDDTIIHKPKLDGDRGDVRLYYAVAWFKDGAAQFEIMSKSDVENHRDKFSKTKNYGPWKENFDAMARKTVLKKLVNQLPMDVEFHEAVQEDETVRKTINDEPEVIAAEYEIIDAPEVVEGNE

SEQ ID NO: 307 - protein, 265 aa, Coprococcus sp.
ANNIDLKQELAEQASKVPAKKDEEVKLTKSMTIPDMVKAMMPEIKKALPAVMTPERFTRIALSALNTTPALNQCTPMSFLAALMNAAQLGLEPNTPLGQAYLIPYKNHGTLECQFQIGYKGLIELAYRSGQMQTIQAQTVYENDEFAYQYGLEPVLVHRPAYSDRGEVKYFYGIFKTVNGGYGMAVMSRAEMDLYAKTYSKAYDSSYSPWKSNYEDMAKKTVIKQALKYAPIKTDFQRALSFDETIKKEISLDMSTVKNELLDVA

SEQ ID NO: 308 - protein, 271 aa, Butyrivibrio sp.
PYLFGGQMKEQEIKNQLAAKAVETTNPKLSKNMNIADLIKAIEPEIKKALPTVITPERFTRIALSALNTTPKLAECSQMSFLAALMNAAQLGLEVNSPLGQAYLIPYNNKGKLECQFQIGYKGMLGLAYRNPEIQTIQAQVVYENDDFKYELGLDSKLYHKPSLSDRGKVRCYYALYKLRNGGYGFEVMSRRDVEEYAKRYSKVTDSLYSPWANNFDSMAKKTVIKQLLKYAPLRTDLEKAMSMDESIKTRVSVDMSEVENEETFDAEVEV

SEQ ID NO: 309 - protein, 271 aa, Staphylococcus equorum
ATNETLKQKVVERKPNGVKEQSPKTQLNHLLKKMAPEIQRALPKHMDSDRMARIAMTAVSNTPKLLECDQMSFIAALMQASQLGVEPNTGLGQAYLIPYAGKVQFQLSYKGLIDLATRSGQYKSIYAHEVYTNDEFEYRYGLFKDLIHIPSQEPEGNPIGYYAVYHLKNGGYDFVYWTRERVDKHAKEFSQAVQKGWTSPWITNYDAMAKKTVLKEVLKYAPKSIEMNKAVENDSTIKEEIDKDMSTVIDVTDYSEVEEQESLETGGQTSK

SEQ ID NO: 310 - protein, 271 aa, Hungatella hathewayi
RRDRNVTAVKQELEKKAAGTSQAVKLTKNMTIVDMVKALEPEIKRALPSILTPERFTRMALSAINNTPKLAECTPMSFIAALMNAAQLGLEPNTPLGQAYLIPYKNKGVLECQFQLGYRGMIDLAYRNERMQSIEAQTVYEHDEFFYELGLHPALVHRPTFEDRGEIRAFYAIFRLDNGGYRFEVMSKSYVDAYAMRYSKAFTSEFSPWKSNYEGMAKKTVIKQLLKYAPVKSEFQKAITLDETVKTELSVDMSEVQNEDLSETLTAESAA

SEQ ID NO: 311 - protein, 264 aa, Roseburia intestinalis
GDIRSELAKKAEQTQGNTKLTKSMSIADLIKAMEPEIQKALPSVITPERFTRMALSALNTTPKLQECTPMSFLAALMNAAQLGLEPNTPLGQAYLIPYKNKNVLECQFQLGYRGMIDLAYRNGHMQSIEAQAVYENDVFSYALGLHPELVHKPTLEEKGALKAFYAIFRLDNGGFRFEVMGKTYIDWYANRYSKAFTSEFSPWKSNYEGMAKKTVIKQLLKYAPLKTEFQRALSTDETIKNSLNVDMGEVLSEDIIDMPCEEVA

SEQ ID NO: 312 - protein, 264 aa, Staphylococcus chromogenes
ANAKEFKKQMNSKNEVAETNNAPQKAKGPRQQVSDLLDRMAPEIQKALPNNMSAERMARIAMTAVSSNPKLLECDPKSFIGALMQASQIGLEPNTALGQAYLIPYGNQVQLQLSYLGLIELATRTGQYKAIYAHEVYKDDEFSYEYGLYKNLIHKPVDDPNGEPIGYYAVYHLMNGGYDFAYWTRKKVEAHAQQYSKAVQQGWNSPWKSDFNAMAKKTVLKDLLKYAPKAIEVSQAIGSDSKVSEINDEGEIIDVTDYSQEEEK

SEQ ID NO: 313 - protein, 272 aa, Romboutsia weinsteinii
TNLKNTLKNKEAKGNNLAINPSYAMKQLMIKMKGEITSALPKELCSERFQRVALTAFNSNPKLQNCAPMTFIAAMMQSDQLGLEPNTPLGQAYLIPYKVKGIDKVQFQIGYKGLLELAHRSGRLKTLYAHEVRENDEFDIDYGLEQRLIHKPLLKGNRGEVIGYYAVYHLEHNGYSFVFMTYDEVLEHGKKYSKSFEGGIWEKEFDSMAKKTVIKKLLKYAPLSIEIQKAINFDESVKGSIDSDMLLVDKADESIDVEGNVLNQRGIKYGCI

SEQ ID NO: 314 - protein, 264 aa, Roseburia sp.
DVKEELAKMAEEKPTKKLTKSMSIQDMIKVIEPEIKKALPSVLTPERFTRMALSAINNTPKLAECSQISFLAALMNAAQLGLEPNTPLGQAYLIPFQNKGKLECQFQIGYKGIIELVYRNPLIQTIQAQVVYENDEFEYELGLNSRLFHRPALYDRGETVLFYALFKMSNGGYGFEVLSKQDMDAYAKRYSKGISSEYSPWKSNYEEMAKKTMIKKVLKYAPIRTDFQKAVSMDESIKKELSVDMSEVSNENIIDMEEITQEEE

SEQ ID NO: 315 - protein, 267 aa, Mobilisporobacter senegalensis
KDIKSALEKKVDKQDVKLTKSMSITDMIKALEPEIKKALPSVITPERFTRMALSAVNNTPKLAECSQMSFLAALMNAAQLGLEPNTSLGQAYLIPYQNKGKLECQFQLGFKGMIDLVYRNEKVQTIQAHCVYEEDYFEYELGLDSKLAHKPALANRGKMILVYAFFKLENGGFGFEVMSKEDIDIHALKYSKGYSSQYSPWKSNYEDMAKKTVIKKVLKYAPLKIDFQRAISVDETVKAEISIDMSEVQNEEIIDGQCTDVGEIEEK

SEQ ID NO: 316 - protein, 270 aa, Staphylococcus felis
ANANSFKEQVSNKNEVSENNNTPQQKTKGPRQQVSDLLERMAPEIQKALPSHMSAERMARIAMTAISSNTQLLECNPRSLIGALLQASQIGLEPNTALGQAYLIPYYNRNKGEFEAQLQLSYLGLIELATRTGQYKAIYAHEVYKEDEFYYEYGLHKNLVHKPVDDPKSEPIGYYAVYHLQNGGYDFSFWTRNKVELHSGQYSKAVQKGWNSPWKTDFNAMAKKTVLKDLLKYAPKSVEVSRAVGTDSKVSEISQNGEIIDVTDYSKEEE

SEQ ID NO: 317 - protein, 271 aa, Corynebacterium liangguodongii
KDLETRMAANQQPAQQRPTTLADQIRGMEQQFALAMPKGAEASQLVRDALTALRQAPKLAQCTPQSVLGSLMTCAQLGLRPGVLGHAYLIPFYDRRAGGLVAQLVIGYQGLVELAHRSGQIKSLIARTVYENDVFDVDYGLEDKLVHKPYMGGDKGQPIAYYAVAKFTTGGHAFYVMSHPEMLDYRARFAKSAERGPWVDNFEAMALKTCVRQLSKWMPKSTELATAIAADESVRVDLTPDAINYPEHVDGEVVDAQGTTEDTAGEGEQSA

SEQ ID NO: 318 - protein, 271 aa, Oscillibacter sp.
KEGLIQGTQSAQAAKKGPATMQDYIKKMQGEIAKALPSVLTPERFTRITLSALSTNPKLAQTTPKSFLGAMMTAAQLGMEPNTPLGQAYLIPFKNHGVLECQFQLGYKGLIDLAYRSGEVSTIQAQTVYENDEFEYELGLEPKLHHVPAKGERGEPVYFYAVFRTKDGGYGFEVMSVDDVRTHAKKYSKAYSNGPWQTNFEEMAKKTVLKKALKYAPLKTEFMRGLTSDETIKTEISEDMYSVPDETVIEAEGYEVDGDTGEVIERPADGQ

SEQ ID NO: 319 - protein, 266 aa, Brevibacillus reuszeri
SNKLAQRAGQQTQPVKPDQQISALLKRMEPEIARALPKHLTSDRLARIAMTSIRQNPKLLACDQMSLLAGVMQSAQLGLEPNTPLGEAYLIPYGKEAQFQVGYKGIISLAHRTGEYQAIYAHEVFKNDEFSYSYGLDKTLNHKPADEPEGDPIYFYAVYRLKNGGFDFVVWSTKKIDAHAKKYSQAYQKGWTTPWKTDFVAMAKKTVLKEVLKYAPKSAEMAKALVMDETVKNEISEDMSEVPGMVIDIEADAANVEETAGGGASE

SEQ ID NO: 320 - protein, 269 aa, Mammaliicoccus sp.
ATNESIKNQVASRKKNEVQNKSPKTQLNDLLIKMGPEIQRALPKHMDADRMARIAMTAVSTTPKLLECDQMSFIGALMQASQLGVEPNTGLGQAYLIPYGGKVQFQLSYKGLIDLATRSGQYKAIYAHEVFPNDEFNYQYGLFKNLEHIPSQEPEGEPIGYYAVYHLKNGGYDFVYWTRERVDKHAKDFSQAVQKGWTSPWKTNYDAMAKKTVLKEVLKYAPKSIEMNKAVNSDSTIKDEINEDMSSVIDITDYEEVNDQQEEKKEESK

SEQ ID NO: 321 - protein, 261 aa, unidentified
  source 1..261: note = Clostridiaceae bacterium
SNLKKALKTNETKGNSVTVSKAYAMKQLMIKMKGEITSALPTNLSSERFEKVALTAFNSNPKLQKCDPRTFIAAMMQSAQLGLEPNTALGLAYLIPYEVKGINKVQFQIGYKGLLELANRSGKLKTLYAHEVRENDEFDIDYGLEQKLIHKPLLKGNRGNVIGYYAVYHLEPSGYNFVFMTYDEVLEHGKKYSKSFEGGVWEKEFDSMAKKTVIKKLLKYAPLSIEMQKAIVFDESVKGSIDSDMLLVDKEDESIEGSELN

SEQ ID NO: 322 - protein, 268 aa, Staphylococcus intermedius
ANANSFKEQVSKNEVQETNNEKPKGPRQQVSDLLERMAPEIQKALPSHMSAERMARIAMTAISSNPQLLECNPRSLIGALLQASQIGLEPNTALGQAYLIPYYNHKKKEFEAQLQLSYLGLIELATRTGQYKAIYAHEVYKEDEFYYEYGLHKNLVHKPVDDPNGEPVGYYAVYHLQNGGFDFAYWTKNKIELHAGNYSKAVQKGWNSPWKTDFNAMAKKTVLKDLLKYAPKSIEISQAVGSDSKVTEINKQGEIIDITEYGQEALEG

SEQ ID NO: 323 - protein, 269 aa, Corynebacterium urealyticum
AKNLEARMQQSTNAPARADKPLSLPDQIRQMEDQFRLAMPKGAEATQLVRDALTCLRQTPQLAQCTPASVLGGLMTCAQLGLRPGVLGHAYLIPFNDRRSGNSVAQLVIGYQGLVELAHRSGQIKALIARTVYENDHFDVDYGLEDKLVHKPHMGADKGNPVAYYAVVKFTTGGHAFYVMSHPEMLQYRDKNAKSPKRGPWVDNFEAMAHKTCVRQLAKWMPKSTEFSQALATDESIRLDVTPDAINYPDHPAEGEVIDGEVEQDGGQQ

SEQ ID NO: 324 - protein, 270 aa, Staphylococcus nepalensis
ATQNQFKNQLTQKKENNNQPQQKAVGPKQEISNLLDRMAPQIQKALPQHMSAERMARIAMTAVSSTPKLLECDPKSLIGALMQSSQIGLEPNTNLGQAYLIPYGKEVQLQVSYLGMIELANRSKQYKAIYAHEVYPEDYFEYQYGLQKDLIHKPADNPQSEPIGYYAVYHLLNGGYDFVYWSKAKIDDHARQFSKAVQKGWQSPWKTNFNAMAKKTVLKDLLKFAPKSIEMNNAVSSDSKAQQIDDDGNIIDVTDYSQVNDEPEQLQEGQ

SEQ ID NO: 325 - protein, 259 aa, Bacillus sp.
ATNESLKNQITNKKTGEVPLTPAQQVSSYLKAYEGTFQQIAPKHFNTERFQRIALSEIRKNPKLLECSVPSLMSAVLQSVKLGLEPGLFGQAYLIPYGKEVQFQIGYRGLIELSQRSGRILKIQAREVYENDEFEVSYGIDDNIIHKPALDVDRGKVRLYYAVAWFKDGGAQFELMSISDVEKHRDKFSKTAKFGPWKDHFDEMAKKTVLKKLVKQLPMDVEFQEAVQEDETVRKTITDEPEILQAEFEIVDQPEISVE

SEQ ID NO: 326 - protein, 261 aa, Pseudoflavonifractor sp.
ATEKAIQRATGRAPALENRPALQQYIKQMSGEIKKALPSVMTPERFTRIVLSALSTNPKLAETTPQSFLGAMMTAAQLGLEPNTPLGQAYLLPYWNSKANAYECQFQLGYKGLLDLAYRSGEISVIQAHVVYSEDQFSYSFGLKPELKHIPAGEERGEPVYVYAIFHTKDGGYGFEVCSIDDIRAHAQRYSKSFQNGPWQTNFEEMAKKTVLKRVLKYAPLKSEFLRGLAQDETIKQEISEDMYMVEAAYAEPDVSSAEND

SEQ ID NO: 327 - protein, 272 aa, Nakamurella lacteal
ASNLAARAAEQVEQQTAPNRPPTIKEQIGRMESQFALAMPRGSEAAQLVRDAITAINTNPQLAECTPASVLGALMTCAQLGLRPGVLGHAWVLPFRSKGVMQAQLVIGYQGLVELAHRTGQVASLIAREVHERDHFDVDYGLADSLIHKPLLNGDRGPVTGYYAIVKFKGGGHSFIYASKADVEAHRDKFSKMKSFGPWVDNFDSMALKTVVRMLAKWMPKSTEFANAISADEGVRVDYSPTADVAQATEYVQPQLEEAPVEGVVVSEGGES

SEQ ID NO: 328 - protein, 266 aa, Clostridium hathewayi
ANDIRGELARRASGTETQAVKLTKNMSIPDMIKALEPEIKRALPTILTPERFTRIALSAINNTPKLAECSPMSFIAALMNAAQLGLEPNTPLGQAFLIPYKVKGSLECQFQIGYRGMIDLAYRNERVQSIEAHTVYENDVFEYELGLNPRLVHIPTMEEPGDPIAFYGIFRLDNGGFRFEVMNKNAIDAYAARYSKAYDSASSPWKNNYESMACKTVLKQLLKYSPMKSEFQKAVSMDESVKTELSVDMSEVQNVNLIEETQEDAA

SEQ ID NO: 329 - protein, 266 aa, unidentified
  source 1..266: note = Veillonellaceae bacterium DNF00626
KTTGGLQQQQQQQAQALQNGGTTLKGYLQAMMPEIKKALPTVMTPERFTRIVMTTISTNPALQNCTPQSFLGAVMQAAQLGVEPNTPLGQAYLIPYGNQVQFQLGYKGLIDLAYRSGEVQSLQAHEVYQNDTFEYELGLNPKLKHIPALTNRGDVILYYAVIKFKNGGEGFEVMSKEDVEAFAKSKSKTYGRGPWQTDFDEMAKKTVLKKVLKYAPMKTDFIRAVATDETVKSSVAEQMADLPDETVTIDTEAQVVVDKETGEVKS

SEQ ID NO: 330 - protein, 261 aa, Solobacterium moorei
TEIKAAKAPATVAKAGVSTQNKTIKDYITIMKPEIEKALPSTITPERFTRITLSAVSNNPKLQACSPSTFLSAMMQSAQLGLEPNTPLGQAYLIPYGNSCQFQLGYKGLLQLAYNSGQIKTIRTETVYENDEFKYELGLHSDLVHVPAMSNRGNPTAYYAVIEYTNGGYGFEVMSHDDVLEHAKKFSKTFNNGPWQSDFESMAKKTVLKQALKYAPLSTELVSKINTDETVKSSISDHMEEVKNDIDLSQIIDAETGEIHE

SEQ ID NO: 331 - protein, 268 aa, Helcococcus sueciensis
AQAKELLENKTNNTVKKSEKQTMENLLTLMADEIKKALPENVKSERFRRIALTAFNGNKDLQQCEPTSFLAAMMQSAQLGLEPNTPLGQAYLIPYNNSKKNIKEVQFQVGYKGMLDLAHRTNQYKNIQANIVYEKDEFDIEYGLNPKLKHIPNMKEDRGQAIGYYAVYNLINGGQGFEYMTRAEVEKHAQKFSKTYRNGPWQTDFDEMAKKTVLKKVLKYAPMSTELQEATAIDERVVNEENIKSKNEDKFVDVDWSYVDDVEEDVIE

SEQ ID NO: 332 - protein, 271 aa, Clostridium sp.
AARATNSVKEELAKKAETKAVGEKKLTRSMSIADLIKAMAPEIKKALPEVITPERFTRMALSALNTTPKLQECTQMSFLAALMNAAQLGLEPNTPLGQAYLIPFNNKGTMECQFQIGYKGLIDLGYRNPQMQIISAQAVYENDEFEYELGLNPKLEHRPALHDRGELRLFYGLFKLVNGGFGFEVMSKEAVDAYAKEYSKSFDSSFSPWKTNYEAMAKKTVIKQALKYAPIKADFRKALSTDETIKNEIAEDMSEIHGEDIFDAEYTEQTA

SEQ ID NO: 333 - protein, 265 aa, Clostridium fimetarium; identical to SEQ ID NO: 238

SEQ ID NO: 334 - protein, 269 aa, Robinsoniella sp.
TTRTGNIKEELAKKAEGTNGDTRLTKAMSIADLIKAMEPEIKKALPEVITPERFTRMALSALNTTPKLRECTQISFLAAMMNAAQLGLEPNTPLGQAYLIPFNNKGTMECQFQIGYKGMIDLSYRNPQMQMISAQAVYENDEFKYELGLNPTLIHRPVLRGRGEVILFYGLFKLTNGGYGFEVMSKEEMDAYAKAYSKAIDSSFSPWKSNYNGMAKKTVIKQVLKYAPIKADFRKALSSDETIKNEISENMSEIHGEIIFDTDYMEESA

SEQ ID NO: 335 - protein, 264 aa, Blautia sp.
NVKEELAQKAEITQKEVKLKKSMSISDMIRALQPEIKKALPSVVTPERFIRMALSALNTTPKLAECSQISVLAALMNAAQLGLEPNTPMGQAYLIPFNNKGKMECQFQIGYKGLLELVYRNPAIQIIQAQTVYENDYFEYELGLNSRLIHRPELEDRGEIRLFYGLFKMVNGGYGFEVMSRQEMDQYAARYSKSFASGFSPWENNYEDMAKKTMIKRVLKYAPVKIETARALINDESIKLHLSEDMSEVENETVVDGQAEEKAA

SEQ ID NO: 336 - protein, 271 aa, Ruminococcus sp.
GTSIQKNVENNALQKEKMPTMQAYIKKMEGEIKKALPSVMTPERFTRITLSALSTNPKLAATTPGSFLGAMMTAAQLGLEPNTPLGQAYLIPYSNKGKLECQFQIGYKGLIDLAYRSGSISVIQAHTVYENDDFEYELGLDPKLKHIPSKSADKGNPAWFYAVFKTKDGGYGFEVMSIEDIRSHAAKYSQSYNSAYSPWKTNFEEMAKKTVLKKALKYAPLKSDFVRQISTDETIKTKLSDDMFSVPAETIEVEGIEVDTETGEITEVDHA

SEQ ID NO: 337 - protein, 253 aa, Romboutsia lituseburensis
SNLKNVLKNQEDKGQGITVNPTYAMKQLMIKMKNDIDLALPKNLSSERFQKVSMSAFNNNEKLQNCEPTTFIAAMMQSAQLGLEPNTPLGQVYLIPHNLNGVDKVQFQVGYKGLLQLAHRSGKLKTLYAHEVKENDEFEIDYGLEQKLIHKPLLKGNRGDVIGYYAVYHLEPSGYSFEFMTYDEVAKHGKKYSKDFEGGIWEKDFDSMAKKTVIKKLLKYAPLSIEMQKAVAFDESVKSSIDSDMLLVESIGE

SEQ ID NO: 338 - protein, 265 aa, Streptomyces showdoensis
TSDARNAVARRAANVGQVEQAGEQPKPTMAQQIERMKPEIARALPKHMDADRIARIALTLIRKNPDLANCTTESFLGALMTCSQLGFEPGSPTQEAYIIPRKGQAEFQLGYQGMVTLFYQHPMASSVKVETVRENDYFEHEEGLEERLIHRPFADGPRGKAIAYYSVARLINGGRTFKVMYPAEIEERRQKLPSKNSPAWRDNYDEMAKKTVLRNHFKALPKSAELARALAHDGTVRTDWQPDAIDVPPEYLSEPQRPELEAGAQ

SEQ ID NO: 339 - protein, 270 aa, Dorea longicatena
TVGKTDEIKQELARKVENTKAGTKLKKSMSIADMIKVMEPQIKKALPEVITPERFTRMALSALNTTPKLNECTPMSFLAALMNAAQLGLEPNTPLGQAFLIPYNNKGKMECQFQLGYKGLIDLSYRNPNMQIITAHTVYENDEFEYELGLNPCLDHRPTLGERGEIRLFYGLFKLTNGGFGFEVMSKTAMDDFAKEYSKAFDSSFSPWRTNYESMALKTIIKKALKYAPLKSEFRNALSTDETIKNEIGADMSEINSENIFDTVYQEECA

SEQ ID NO: 340 - protein, 262 aa, Streptomyces indicus
STDARNAVARRAETVGQVEQQAQQQPTLAQQIERMKPEMERALPKHMSADRMARIALTLIRKNPDLATCNTQSFLGALMTCSQLGFEPGSPTQEAYIIPRKGNAEFQLGYQGMVTLFYQHPMASSIKVETVRENDYFEHEEGLEERLVHRPCATGPRGRAIAYYSVARLINGGRTFKVMYPDEIEERRQKLPSKNSPAWRDNYDEMAKKTVLRNHFKALPKSAQLARALAHDGTVRTDATADVIDVAPEYPQRPELEAGPTA

SEQ ID NO: 341 - protein, 265 aa, Staphylococcus pettenkoferi
ATQKDFKNQISQKETQQKQEVQKKKKGPRQQVSDLLDRMAPEIEKALPNHLSADRMARVAMTAVSSNPKLLECDPKSFIGAVMQSAQLGLEPNTALGEAYLVPYAGKVNFQLSYLGLINLATRSGQYKAIYAHEVYAEDEFRYQYGLHKDLIHKPVDNPKGKPIGYYAVYHLLNGGYDFVYWTTERIQKHAKKYSFAVQKGYQSPWNDEFDAMAKKTVLKDLLKYAPKSIEMNNAVRSDDKQSELSDEGVVIDVTNYDEENGEEK

SEQ ID NO: 342 - protein, 265 aa, Tyzzerella nexilis
AGVKEELAKKAESTKGETKLTKSMSIADLIKAMEPEIKKALPEVITPERFTRMALSALNTTPKLRECTQMSFLAALMNAAQLGLEPNTPLGQAYLIPYNNKGVMECQFQIGYKGLIDLSYRNPQMQIISAQAVYENDDFSYELGLNPKLEHCPTLGERGEVRLFYGFFKLVNGGFGFEVMSKTAMDEYAKEYSKAFDSSFSPWKSNYIGMAKKTVIKQALKYAPLKTDFRKALSNDETIKTELSDDMSDIHGEEIWDVEYQEKTA

SEQ ID NO: 343 - protein, 266 aa, Hespellia stercorisuis
ADIKEELAKKVAEGTEDKKKLTKSMSIADLIKAMEPEIKKALPEVITPERFTRMALSALNTTPKLKECTQTSFLTALMNAAQLGLEPNTPLGQAYLIPYKNKGNLECQFQIGYKGLIDLSYRNRQMQIIQAQAVYENDEFEYELGLNPVLVHRPALQNRGAVKLFYGIFKLTNGGFGFEVMSKADMDAYAKEYSKAFDSSFSPWKSNYIGMAKKTVIKQAIKYAPLKTDFRKALSTDETIKTEFCEDMSEVQCKDIWDTEYKERSA

SEQ ID NO: 344 - protein, 266 aa, Roseburia sp.
DVKNELAKKAENTGKVKLTKSMSIADMIKTLEPEIARALPSVITPERFTRMALNALNNTPKLAECTQMSFLAALMNAAQLGLEPNTPLGQAYLIPYKNKGVLECQFQIGYKGMLDLVYRNEMVQTVQAQVVYQNDEFHYALGLTGRLEHIPTLRDRGEPYAFYALFKLENGGYGFEVMSKTDMDAFALQYSKGISSEYSPWKTNYIDMAKKTVIKKVLKYAPLKTEFQRALSNDETIKTHFAVDMSEVEPETVIDMEEGELLESAS

SEQ ID NO: 345 - protein, 267 aa, Absicoccus porci
TTTNQQGMITKKANNSVAKKTNRTMKDYITMYQGEIAKALPSVMTPERFVRIATTAVTNTPKLASCTPQSFIGALLNAAQLGLEPNTPLGQAYLIPYGNQCQFQIGYLGMVELAQRAGTNVDAHVVYANDEFDYSLGLHPDIKHVPAMKDRGEAIAYYAVWHNGENFGFEVMSREDVEKHMKKYSKTYSNGPWKTEFDEMAKKTVLKRALKYAPKKTDLARAVMQDETIKQFNPKADNDMADAKNDFFDVEYDEVDENTDPVTGEVK

SEQ ID NO: 346 - protein, 181 aa, Geomicrobium sp.
GYKGMIDLARRSGHIKSIYAHTVHANDEFEYELGLEPKLVHKPATGDRGNMEYAYAVAHFVDGGYQFEVFSHHDIEQVKKRSKAGNFGPWKTDYEEMAKKTVVRRMFKYLPISIEIQQHASQDETVRRDITEEAEKVDNIIDLPNYEDPNNIDVPDEEQDEQKDEKQKQQGSAEEIALDFK

SEQ ID NO: 347 - protein, 235 aa, Streptomyces sp.
STNLAARVEARRQNPTTKQPARRGKAAQQPTLVQFVQSMRGEIARALPSHVASPERIARIALTELRRVDHLAECTQESFGGALMTCAALGLEPGGVGGEAYLLPFWNKKVRAYEVTLVIGYQGMVRLFWQHPAAAGLAAHTVHEGDEFDFEYGLEPFLRHKPARTGRGKPTDYYAVAKMANGGSAFVVMNVEDIEAIRHRSKARDAGPWSTDYGLRRHGAQDLHSAVVQVAAEVC

SEQ ID NO: 348 - protein, 271 aa, Acetoanaerobium noterae
SNLKNELAKKANNSVTDGNKEPQTIKDWIKVMEPAIKKALPSVITPERFTRMALTAISVNPKLAECTPKSFMGSLMNAAQLGLEPNTPLGQAYLIPYKNKGNMEVQFQIGYKGLIELAYRSGEFANIYAKEVFENDEFEYEFGLEPVLKHKPASGNRGEVIAYYAVFKLTNGGFGFEVMSKEDITNHAKTYSQAYSSSYSPWSKNFDEMAKKTVLKKVLKYAPIKVEFVKQIVQDSTIKTEINSDMTEVESQNVFEAEETDYEVIDQEETK

SEQ ID NO: 349 - protein, 269 aa, Staphylococcus haemolyticus
ATQNEFKNQLAKKEDKGNTNAPTQTKSTNPRTIAQNYLAKMKPEIEKALPAHMSHERMTRIALSAVNSNPELTEVILNNPTSFLGALMQSAQLGLEPNTNLGHAYLIPYYDKNSGKKIVNLQLGYMGLLDLAHRSGMYQKIFAMPVYKDDYFEYQYGTNEKLNHVPAQVSKGEPIGYYAFYKLTNGGVHFVYWSRQKMQMHKDRYTRRGSVWNNNFDAMALKTVIKDVLKYAPKSVEMGEAVQSDENNFEFNEDSKVIDVTDYETEENK

SEQ ID NO: 350 - protein, 271 aa, Staphylococcus hominis
ATANDFKNQVTKKESDNTKESSNKKTELATTSPRQVAQNYLEKMKPEIAKALPAHMSHERMTRIALSAVNSNPQLTEVILNNPTSFLGALMQSAQLGLEPNTSLGHAYLIPYNFKGKKIVNLQLGYMGLLELAHRSGLYKKIFAMPVFKDDFFEYQYGTNEKLNHIPAQVQNGDAVGYYAFYQLTNGGVHFVYWSRQKMERHKDLYTRKGSVWNTNFDAMALKTVIKDVLKYAPKSVEMSSAVQSDNSNFEFSEDSSTVIDVTDYETEDNK

SEQ ID NO: 351 - protein, 206 aa, Lacrimispora sphenoides
ADVKQELEKRAAGSGGQSVKLTKNMTIVDMVKALEPEIKRALPCILTPERFSRMALSAINNTPKLGECTPMSFIAALMNAAQLGLEPNTPLGQAYLIPYKNKGVLECQFQLGYRGLIDLAYRNERMQSIEAQVVYDNDEFSYELGLHPSLIHRPTFDEPGEIQAFYAIFRLDNGGFRFEVMSKNYVDSLCHALFKSIYFRFQSLEK

SEQ ID NO: 352 - protein, 232 aa, Clostridium sp.
DFKEELAAKAEVAATTKKSDGVKLTKNMSIVDMIKALEPEIKRALPSVLTPERFVRMALTAVNNTPALAQCTPMSFIAALMNAAQLGLEPNTPLGQAYLIPYKNKKKGVVECQFQIGYKGMIDLVYRNDNVQTIQAHIVRENDHFEYELGLESKLRHIPAMEGRGEMMYVYALFKLTNGGYGFEVLNKEAVIAHAERYSPSYDGFSPWKTDFESKGLELFLILDLSSKQSGK

SEQ ID NO: 353 - protein, 218 aa, Streptomyces scabiei
DADRMARIALTLIRKNPDLATCSGESFLGALMTCSQLGFEPGSPTQEAFIVPYKGEATFQLGYQGMVTLFYQHPMASSVKVETVRENDYFEHEEGLEEKLVHRPCKTGPRGKAIAYYSVARLINGGRTFKVMYPAEIEERREKLPSKNSPAWRNSYDEMAKKTVLRNHFKALPKSAELARAMAHDGTVRTDWQPDAIDVPPEYLSEPQRPELGTGSTQ

SEQ ID NO: 354 - protein, 262 aa, Pirellula sp.; identical to SEQ ID NO: 227

SEQ ID NO: 355 - protein, 268 aa, Bifidobacterium castoris
GALATTAKNNELTTMNTMGDIHALIRGRRAQIESVMSGVLTPERLYSLLQSAVSHEPKLLQCTPESIVACCMKCAVLGLEPSNVDGLGKAYILPYGNKNYQTGQVEATFILGYKGMIELARRSGEIKSLNVTPVFEDDGIKLFMDEAGQPYIKAGEVNPLANHTPDKLMFVFLNAEFTNGGHYRTYMTRAEIDAAKKRSSAGDRGPWKTDYVAMARKTVVRRAFPYLPVSTEAQSAAVEDETTPHFDFLDRNTTPVGEPSDVMQEATA

SEQ ID NO: 356 - protein, 256 aa, Staphylococcus warneri
ATQNDFKNQITDKKENKPQQSTNPRQVASDLLERMKPEIAKALPAHMSQDRMTRIALSAVNSNPKLSEVILNNPTSFLGALMQSAQLGLEPNTNLGHAYLIPYGNIVQLQLGYLGLLELAYRSGKYQKIMAMPVYKDDFFEYQYGTDEKLNHIPAQQQTGDAVGYYAFYKLTNGGTHFVYWSRQKMNMHQQQYSKGGNVWRNNFDAMALKTVIKDVLKYAPKSIEMGEAVTSDNNNFDFKDGGDIIDVTDYETEEN

SEQ ID NO: 357 - protein, 264 aa, Propionibacterium freudenreichii
TQQMPIKAQGEPTKELQQKAAVDRFNATLHQMQNEIARALPKHMTGDRFVRIVLTEVRKNPTLALCDPLTMFGSLLTAAALGLEPGLNGECWLVPRKNHGTLEAQLQVGYRGVVKLFWQNPAATYLDTGYVCERDEFRFAKGLNPILEHTPAEGDRGKVVRYYAVAGLNTGARVFDVFTPAQIKTLRGGKVGSNGDIPDPEHWMERKTALLQVLKLMPKSTQLAAVPAADGRAHTISDAQQIFGGVDPTTGEVLDAEPVEDGAA

SEQ ID NO: 358 - protein, 260 aa, Asaia sp.
SNALATPTEKLRTQITSMTGEFRNALPSHIKPEKFQRVVMTVVQQNQGLMNADRKSLLASCLKCAADGLIPDGREAALVMFGQQVQYMPMLAGIQKRIRNSGEIASIQAHVIYENDHFIWHQGIDASIEHRPLFPGDRGKAIGAYAVAKFKDGSDPQFEVMDVAAIEKVRAVSRAGKSGPWVQWWDEMARKTVFRRLSKWLPMDTEAEDLMRRDDENDAQDVAAPTIRVEAEAPSKLDALEHDDDGVVLEETRELEGSAA

SEQ ID NO: 359 - protein, 268 aa, Microbacterium laevaniformans
TDLSTVAAAAKQNPTMKDLVEAQLPAIERQLGGTMNSDAFVRAVLSEITKSPDLMQADPKTLLGGVMLAAQLRLEIGSGLGEFYLTPRKDHGRMICLPIVGFQGMVKLALRSEFVTNVQAFIVREGDDFTYGANAERGMFYDWTPKDFEEKRPMVGVVATARMKQGGTTWAYLTREQVEDRRPSYWQKTPWGSHPDEMAKKTAVRALAKYLPKATDLGRAIEADEQKVQHVKGLDEVTVTRLDDEPETVVVQETTDAWAATPVAEVQP

SEQ ID NO: 360 - protein, 272 aa, Microbacterium sp.
SKDLSTAAAAAKSQPTMKDLVEAQLPAIERQLGGAMNSDAFVRAVLSEIGKSPDLMNADPKTLLGGVMLAAQLRLEIGSGLGEFYLTPRKDRGRQICLPIIGYQGMIKLALRSEYVLNVQAFLVREGDDFTYGGNSERGMFYDWTPKDFEESRPWIGVVATAKMRGGGTTWVYLTRTQVIDRRPSYWASTPWKTNEDEMVKKTAVRALAKFLPKSTDLGRALEADEAKVQHLKGVDEVQVTRLDDDAETFVVQEQDPMSRTPEEQAEDEANR

SEQ ID NO: 361 - protein, 272 aa, Streptococcus pyogenes
SDLSVAAAAAKTQPTMKDLVEAQLPAIERQLGGAMNSAAFVRAVLSEIGKSPDLMAADPKTLLGGVMLAAQLRLEIGSGLGEFYLTPRKERGRQICLPIIGYQGLIKLALRSEFVMNVQAFLVRQGDQFSYGANAERGMFYDWVPQDFEETRDWIGVVATARMRSGGTTWVYLTRTQVIDRRPSYWNSTPWKTNEDEMVKKTAVRALAKFLPKSTDLGRALEADEAKVQTLRGLDEVEVTRLDDEADTVVVQEQNPMSRTPEEQAEDAEAQR

SEQ ID NO: 362 - protein, 256 aa, Mesotoga sp.
KASEIASMVKKEDERRNHKPDPLAGIVKNLTSIKGEIANALPDAGITPERMIRIVVTLLRQNKSLAEAAMQNPASLLGAVMMAAQLGLDPTNGLDQCALVPRKGKVCFDIMYEGLVELGYRSDRMESIVARTVYEKDTFSLKYGLNEELVHIPYLDGDPGESKGYYMVGKLKGGGNIIVYMTKEQVHKIRDRYSVAYKAGLSGSRKDSPWFTSEDRMGEKTVVKAGFRWIPKSPIIRTALALDETAREASRLPMRN

SEQ ID NO: 363 - protein, 270 aa, Streptomyces sp.
TENTVTAAVAVRDTGPAAQIEAYRDEYAALVPSHINADQWVRLAVGAIRGNEDLTNAARTDIGVFLRELKTAARLGLEPGTEQFYLTPRKSKAHRGQKIIKGIVGYQGIIELIYRAGAVSSVIVESVRANDTFRYVIGRDERPVHEIDWFGGDRGDLVGVYAYATMKDGATSKVVVLNHAQVMQIRAKSDSKHSEYSPWNTNPESMWLKSAVRQLMKWVPTSAEYMREQLRAQAEVAAEQPPAADLPPMPSVELNDEDEAVDAELVDEEA

SEQ ID NO: 364 - protein, 254 aa, Isoptericola dokdonensis; identical to SEQ ID NO: 234

SEQ ID NO: 365 - protein, 271 aa, Gaiella occulta
STAVARRDPVAEVCTTIASKEFEAKIVQALPDGVTPARFVRTTLTAIQQNPDVVKGTRQSLYNAVIRCAQDGLLPDGREAALVVFRAKGTDVVQYLPMIGGLRKIAAEYGIKIETAVVYERDKFEWELGFEPRVLHVPPALGEDRGEPIGAYAVATDKLGRKYVEVMSRQEIEEVRKVSRAATSEYGPWVKWWAEMARKTVGRRLFKQLPLHDLDERGERVISASDAEISFSPSGLDSLPHVDPSEPEEVLTGDVMDDDDDDGIPFGEPAA

SEQ ID NO: 366 - protein, 247 aa, Arsenophonus sp.; identical to SEQ ID NO: 256

SEQ ID NO: 367 - protein, 250 aa, Streptomyces sp.
VEHYKADLAQVMPSHVKPDTFIRLAVGVLRRDRNLAQAAQNNPAALMGALMDAAQLGLTPGTEQFYLVPRKKAGRLEVQGIRGYQGEIELIYRAGAVSSVIVEVVRQADTFRYSPGRDERPEHEIDWDAEDRGPLRLVYAYAVMKDGATSKVVVLNRAQVMKAKAMSQGSDSAYSPWQKHEEAMWMKTAAHRLTKWVPTSAEYMREQLRAAAEVAAEHRPTPVAAAPGMPSVPGEDEAIEAEFVDEDEVA

SEQ ID NO: 368 - protein, 266 aa, unidentified
  source 1..266: note = uncultured Mediterranean phage uvMED
TSSITPLVAMQGTLEKMADKFTEALPRQMDVNKFISVAKLTLNKNPRLLQADKTSLMQTFMKAAQDGLYLDGKEAAAVQYGQSVQYIPMVEGIIKVLHNSGLIKTISAEVVYENDFFDYELGTAPKITHKPLIVGDRGKPMCVYAVAITTNDGEYYEVMNMDQINQCRQVSKASSSPHSPWVKWFDQMAKKTVIHRIAKRLPKNDAINSVVTVDDEPNFQQAVNVTPSEPKDSLSRLRDSIGMEGKDVEQAANDLLEKYNKEAREE

SEQ ID NO: 369 - protein, 271 aa, Streptomyces sp.
SQISNALATRDQGPAAQIEQYRDEYAALVPSHVNADQWVRLAVGAVRGDEKLMEAAQNDIGLFLREMKTAARLGLEPGTEQFYLTPRKSKPHGGRKVIKGIVGYQGIVELIYRAGAASTVIVEAVRENDTFRYVPGRDDRPVHEIDWFANDRGPLVGVYAYAVMKDGAVSKVVVLNRSRVMEFKAKSDSKHSEYSPWNTNEEAMWLKSAVRQLAKWVPTSAEYRRDQLLAHTETADSVVASVSTAPLPPQPSALDDADPDDDGPIDAELVD

SEQ ID NO: 370 - protein, 262 aa, Streptomyces reniochalinae
SQISNAVAKRDNSPGAMVQQYKADFSTVLPDHVKPDTWVRLAQGVLRRDKNLAQAAERNPGSLMTALLDCARLGHEPGTESFYLVPFGGEVQGIEGYRGVVERMYRAGAIASVKAEVVCQGDDFDYQPDMDKPRHRVDWFGDRGPIVGAYAYAIFKDGSTSRVAVINRAYIDKVKKESKGSDRATSPWMKWEEQMVLKTVAKRLEPWVPTSNEWRREQLRAAREVANEPTPPTTPAPPAPEQVDPDTGEVIDGELVDDTPTQ

SEQ ID NO: 371 - protein, 269 aa, Weissella oryzae
SNNLTSAQYFNAPNIKGKFEEVLGKNANGYVTSLLSVINGSQQLQRAEPSSIMVAAMKAATLNLPIESSLGFAYIVPYGNNAQFQIGYKGLIQLALRSGQIKGLNSGVVYETQFISYDPLFEELEIDFKKPAEGKIAGYFASMKLTNGFSKVVYWTKEQVEQHRDRFSKGKNNGPWKSDFDAMAQKTVMKAMISKYAPLNQEMQQAIVEDSESELTVPRDVTTSNEAAELNSLLTTPKVQEGANTDLSEPFPNAEETQLFDDLASVTGD

SEQ ID NO: 372 - protein, 256 aa, Oenococcus oeni
SNELKTILNAPTTKEKFDEVLGRNAQGYINSVLNAVGNSKLLQNASPNSILSGAMKAATLNLSIDPNLGYAYFVPYGHEAQLQIGYQGLIQLAQRSGQIKILNAAPIYDEQFKSLDPVTGKLTLNKKIVPDTNKKPTGYVAYLKTVTGFEHTEFMSYADIEKFAKRFSKSFNSSTSPWKTDFNAMAKKTLIKQVLKYAPMSIDLQTAVSADNDDIEPKDITPDEDKETVDKISNLISDNKQDDTLSQLEEVANANN

SEQ ID NO: 373 - protein, 265 aa, Pseudarthrobacter sp.
TSQLAEATAAKAVEQRKNPTARDLIQAQQAAIETQLAGAMNSAAFVRAAISSVSASPQLQQATPASLLGGIMLAAQLKLEIGPALGHFHLTPRMVSKKDGDNWVEVWTCLPIIGYQGYIELAYRSGRIEKIESLLVRKGDKFDHGANSERGRFFDWAPADYEETREWTGVIALAKIKGAGTVWAYLPKEKVIARRPDRWEKTPWATNEEEMARKSGIRALAPYLPKSTELGKALEADEHKVEHIAGVHDLVVSKAEDEPLEEPTA

SEQ ID NO: 374 - protein, 268 aa, unidentified
  source 1..268: note = Patescibacteria group bacterium
TNQPTTHVATTPNQRPATTLEQFRHQLVGDYQKQVLNYFNGQKEKAMKFMSAVVYSAQKNPALLECDRTTLLHAFMACAEYQLYPSSVSGEAYVIPYKGKAQFQLGYQGIITLLYRAGVEAVNAQIICENDAFEYEEGLEPNLVHKPNVLKDRGKPIGVYAIAAINGHKLFKVLSEAEVMKFKGFSQSKNSEYTPWNPDNDPELWMWRKTAIKQLSKLLPKNDALQKAISEDNQDSVIEARRSTLDAGGPAVGRALHDPNASNEPEGK

SEQ ID NO: 375 - protein, 267 aa, Actinopolyspora xinjiangensis
TGQTIGTAVAKKDDENPSAIIATNRADLARVMPSHVRTDSWVRIAQGIVRRDKNLAHAARQSPGTLMVALMEAARLGLEPGTEQYYLTPRKNKGKPEVLGIPGYQGLIELMYRSGAVSSVVVETVRENDTFQWAPGRMERPEHEADWFAINGERGQLRGVYAYAIMSNGATSKVVVLNRNDIARARDSAQGADSEHSPWKNHEEAMWLKTAARRLAKWVPTSTEDRRIVQGVAERSDQPTEAPLDLTDEPDTDQPIEGELVDEEATQ

SEQ ID NO: 376 - protein, 260 aa, Mycobacterium sp.
TTQPEYKPVAQGADKQMTTGKLLKMLEPEIGRALPKGMDPDRICRLVMTEVRKNPMLTQCTQESFAGALLTASALGLEPGVNGEAWLVPYRDRKRGIVECQFIMGYNGVAKLFWQSPHADRLDAQLVCANDHFRYVKGLSPILEHVESDGDRGDPIAYYAIVGVKGAQPMWDVFKPEAIAQLRGGRVGTKGDIDDPQRWMERKTALKQVLKLAPKSTRLDLAIRADERSGSDLYKSQGMEVHAIEPGFIETEAEPETQEQ

SEQ ID NO: 377 - protein, 267 aa, Streptomyces acidiscabies
TDNAISNAIATRDNGPEAIVQQHRDDLTLVLPAHHKGETWMRLATGALRRDANLRQTAARNPGSLMNALLECARLGHEPGTESFYLVPFGNEVQGIEGYRGIVERIYRAGAVKAVKAEVVYENDHFRYHPGMDRPEHEPDYFADRGRIIGAYAYGVFQDGSTSRVVVINRAYIDKVKKESKGSDRASSPWVKWEEGMVLKTVARRLEPWVPTAVEWRTEPTPASAAEATAPVGDGVKAIAAPAPTSPYDDEGPIEGEFVDEYDGGAA

SEQ ID NO: 378 - protein, 251 aa, Yersinia pestis
NQVATLESIHADLSSALTRQGIQSLLPSHVSPEQFTRTAATALVADPELQNADRQSLVMSLIRCAQDGLVPDGREAAMVVYNTKQGDQWVKKAQYLPMVDGVLKRARQSGQVANITGKVVHMADKFDYWVDENGEHIEHRPAFENHGEIRLVYAFAKLTSGEIVVEVMSRSEVEKVRDATAKKDRDGKPKVPAVWQKWFDRMALKTVLHRLARRLPCASELYSLLDVNQIADEAEKPAECGAQRESSTTAA

SEQ ID NO: 379 - protein, 265 aa, Rufibacter latericius
SNQLQVAREQVISAQKSFKNVPNNKLDFEREAGFAMQMIQSNPFLASMDANSIRNCIVNVALTGLTLNPVLKLAYLVPRKGKLILDPSYMGLINVLVTSGAAKKIEADVVCENDFFDYEKGTNGFIKHKPSLSSRGEIIAAYAIAHLPNGEVQFEIMNREELEKVRKSSEAAKKGSSPYDGWASEMMRKAPIRRLFKYLPKHNIPDQVINTLSLDEQNNGVDFSAQKQEAFKGKAADFFEDEPANTVDADYTDMSHEEADNELAA

SEQ ID NO: 380 - protein, 272 aa, Streptomyces sp.
SQIGNAIATRDEGPAAQIEVYRDEYAALVPSHVNADQWVRLAVGAIRGNDDLLKAAGNDIGLFLREMKTAARLGLEPGTEQFYLTPRKSKAHGGRPVIKGIVGYQGIVELIYRAGAASTVIVEAVRQNDVFRYVPGRDDRPVHEIDWFGQDRGPLVGVYAYAVMKDGAVSKVVVLNKARVMELKAKSDSKNSPYSPWNTNEEAMWLKSGVRQLAKWVPTSAEFRRDQLLAHTDTADGVIASVSAPPLPPQSAALEDLDPDDEGPIDGELLDD

SEQ ID NO: 381 - protein, 270 aa, Streptomyces sp.
SEISNAIATRDQGPAAQIEAYRDEYAALVPSHINVDQWVRLAAGAIRGNEDLMEAARNDIGVFLRELKTAARLGLEPGTEQFYLTARKSKAHGYALIIKGIVGYQGIVELIYRAGAVSSVIVEAVRANDTFSYVPGRDDRPIHEIDWFGGDRGPLVGVYAYAVMKDGAVSKVVVMNHKRVMEIKARSDSKNSQYSPWNTDEESMWLKSAIRQLAKWVPTSAEYKSEQLRAHAEAIGELASVASAPLPPQPSVLDDVDPDDEGPIEGELVD

SEQ ID NO: 382 - protein, 262 aa, Agromyces sp.
STTVALPAQKAEAVIQQVTGAANGFAAALADRIGPDRFVRAAVTSIRTSPQLAQCEPLSILGGLFVAAQLALEVGGPRGLAYLVPYGREAQLIVGYRGYVELFYRAGARKVEWFIVRDGDTFRQWSTGRGGRDYEWTPLDDDSNRRPIGAVAQIQGAHGEFQFEHMTVDQINERRPKRATSGPWVDWYEEMALKTVMRQLAKTARQSTDDLAFAAANDGAVITQVEGGQARVVHPATSEPEQPLSLDALERTPGELAEETNP

SEQ ID NO: 383 - protein, 155 aa, Clostridium tunisiense
TTKANVTSVKNALKEQIQVQQVAAQTDTSFQGVLTKQLQHQFKAIQSLVPKHVTPERLCRIGINAASRNPQLMNCTPETIVGAIVNCATLGLEPNLLGHAYIVPFYNNKTGKMEAQFQVGYKGALDLIRRTGAVSTLSAHEVYGPRSIFWTQYFY

SEQ ID NO: 384 - protein, 260 aa, unidentified
  source 1..260: note = Rickettsiaceae bacterium
TNSIETNIEDLSPGNQTKTQISETNAPIVSEIRTIKRDGVYDLCSSRREKVLPFLGNNSQKFERLARSFAFEINTNPKLASCDQLSILQAFYKCCEYGLDPASSLQQIWMIPYNGKIDFQIGYKGWLQLLWGSKLITNAYSCAVYQGDQFEYELGLNPNIKHIPQHKSINDVNELIATYGVIKLKSNEVQLRVCWRDELEKSKKSSKSNGREDSPWNRHFEAMALIVPMRKMAKNLALALRAEDFDDEDYVNENNNQGMA

SEQ ID NO: 385 - protein, 260 aa, Francisella sp.
SNLVVAKQCLASAEKSFIGISGDEKKYKRECNFAVQSLQANSYMLQQANANPNSLRNAIINLASMGLTLNPAEKKAYLVPYGGKNPRVDLQISYMGLIDLAISDGAIMWAQAKVVRQNDLFNITGVDTPPEHKYNPFDSEQTRGDVIGVYCVAKFPNSDYITEIMSITDINSIKSRSSGVKSGNTTPWDTDFDEMAKKTVIKRASKYWKGSSKLSKAIDFLNNENNEGINFNKQEEKPKQNINDLMNDDVVDIDSEVGDE

SEQ ID NO: 386 - protein, 266 aa, Streptomyces noursei
TSPIRAAVARRAGDPAALISQYTADFAAVLPSHIKPATFVRLAQGILRRDEKLAQAAANDPGQFMSVLLDAARLGLEPGTEAYYLVPFKGRVQGIVGWQGEIELMYRAGAISSVIVETVREDDVFVWTPGLVDRETPPRWEGPMSYPFHEVEWAGDRGPLRLVYAYAVMKGGATSKVVVLNAQDIERAKKTSQGADSPSSPWRQHEAAMWSKTAVHRLAKYVPTSAEYITAQVRAVRQADALSAPPVEEVVDVELVGDGQEQEARR

SEQ ID NO: 387 - protein, 270 aa, Streptococcus lutetiensis
ANQLQMSHKDFFNRPAVKNKFSEVVSGKSDQFITSLLSVVNNNKLLSKADNNSILNAAMKAATLNLPIEPSLGSAYIVPFKGQAQFQLGYKGLIELAQRSGQYKSINAGVVYKAQFKSYDPLFETLDLDFNQPQDEVIGYFACFELLNGFRKITYWTREEVYNHGKRFSKSFNNGPWKTDFDAMAKKTLLKSIIGTYGPKSVDMQEAITDDNKTEYEKAEPIDVTPQEENLTDLIGETPQEELPIANPETGEIQEEQTALFNQLGDLTDD

SEQ ID NO: 388 - protein, 271 aa, Streptomyces uncialis
SQISTAIATRDNGPAAVVEQYRESLALVMPSHLQQRVGAWIRNTQGLLRRDSKLMEAAQNDVGQFVAVLMDAARLGLEPGTEHYYLVPRWNNKKRATEVTGVRGYQGEIELMYRAGAVSSVIVEVVHTQDQFRFRPGRDARPVHDIDWDLEDRGSLRLVYAYAVMKDGATSKVVVLNRQHIAAARAKSDSAAKDWSPWNTDEEAMWLKTAAHRLTKWVPTSAEYLREQIRAQVAVESEQRPEPLPVAPPPAPGTVDADPDDEGPIDGELVD

SEQ ID NO: 389 - protein, 268 aa, Amycolatopsis circi
ISQTVTTAVAQQKDSSPAALVRKYRTDFATVLPSHIKPETWLRIATGALRRSPQLANAAKRNPSSLLVALLEAARKGLEPGTEQFYLVPRKGKNGPEVLGITGYQGEVELMYRAGAVSSVKVEVVREHDTFAYNPGEHDRPVHEIDWRADRGDLVLTYAYAVMRDGATSNVVVLSADDIAVILKKADGADSPFSPWQWNPKAMWLKSAARQLAKWVPTSAEYVRLPDVPLESLPPAKPLDLPRVDDVVDAEIVEDWPTAPDDTADGAR

SEQ ID NO: 390 - protein, 270 aa, Streptomyces sp.
SQISNAIEKRDQGPGAVIEQYKQELALVAASHVKVDTFARLAVGALRQNPKLAAAAQSNPGSLMSALMTAARLGLEPGTEQFYLRPIKRKGVAEVQGIVGYQGIVELIYNAGAASSVVVEVVRANDQFNYVPGLHERPVHNVDWFGDRGDLVGVYAYAVMAGGATSKVVVLSRTHINRAKAKSDGADSDYSPWRTDEEAMWLKTAARRLGKWVPTSAERLTMPAERTDTVLPVGSAAPALDAADPDEDEGPVDGELEPAGGWPETAQPPQ

SEQ ID NO: 391 - protein, 270 aa, Streptococcus macedonicus
ANQMQVSHKDFFNSPAVKNKLSEVVGGKSDRFIASLLSILNNNKLLSSADNNSILTAAMKAATLNLPIEPSLGFAYIVPYKRQAQFQLGYKGLIQLAIRSGQIKSINSGVIYKAQFKSYDPLFETLEVDFSQPEDEVAGYFATIELLNGFKKLIYWTKERAYNHGKRFSKSFGNSPWQTDFDAMAQKTLLKQIISKYAPLSVELQEAITADNENEDEKAAPIDVTPQEESLSDLIGEAAQEELPAADPETGEIQEEQTALFEQLGDLTDD

SEQ ID NO: 392 - protein, 259 aa, Haloactinospora alba
GQSVTNAVAQRDTSPSGMVGKYRDDFAQVMPSHVNGAGWVRIAQGILRRDAKLAEAARNAPQSLMSALMDAAQQGLTPGTTEFYLVPRKRKGSLEVQGITGYQGEIELIYRAGAVASVVAEIVHEHDTFEWIPGKHERPIHEADWFGNRGTMVGAYAYAVMNSGSTSKVVILNQHDIEKARAMSDGADSSYSPWQKWPESMWLKTAAHRLAKWVPTSAEYRHEQERARARSEDTEIPASPDSDVVHAEIVEENDDEQAT

SEQ ID NO: 393 - protein, 260 aa, Clostridium tyrobutyricum
SDKKMVVLGESHKALSKLLETKQEALPKDFNKARFLQNCMTVLQDTKDIDKCQPISVARTMLKGAFLGLDFFNRECYAIPYGGNLQFQTDYKGEIKLAKKYSFNSIKDIYAKIVREGDDFQESIEDGRQTINFKPLPFNNGEIIGAFAVCLFQDGSMLYETMTKQEIEDIRNNFSKAKNSPAWVKTPGEMYKKTVLRRLCKLIELDFDSVETKKTYDETSEFEFGSANHEVSNFDKDDSNIIEADAEIQDDVQEGDGEDE

SEQ ID NO: 394 - protein, 268 aa, Actinocrispum wychmicini
SQTVTAAVAQRDNGAQALIAKYRTDFAQVLPSHLRPTTFVRLSQGLLRRNVKLAEAAERNPASFLAALLECARLGHDPGTDQFALVPFNDRKRNTVEVVGIEQYQGVIERMYRAGAVRSVKAEVVRAADPFEYAPDVMDRPGHKPNWFADRGELIGVYAYAEFFDGSTSRVVMMNRETVMAHKAKSRGATSEDSPWQAWEESMWLKTAVHELEKWVPTSSEDRRAARDGTADPAPVEVPRVADEVLDADLVEDDHADHPTATPTGDVR

SEQ ID NO: 395 - protein, 269 aa, Companilactobacillus furfuricola
VNNLAKLPIQTLVKEPKIVEKFESVLGNKSAQFVTSLINVVNSNQSLKNVDQMSVVASAMVAASLDLPINQDLGYMWLVPYGGKAQPQMGYKGYIQLAQRTGQYKHLNAVAVYEDEFQSYNPLTEQLDYEPHFKDRDSSEKPVGYVGYFELTSGFEKTVYWTRKQIDDHRQSFSKMSGKSKPSGVWATNFDAMALKTVLRNLISKWGPMSVEMQKAYESDEHATTISANDIKDIEVQEQEPATDVSQLINGSATEVNVNDSTTNSKDSE

SEQ ID NO: 396 - protein, 272 aa, Streptococcus parauberis
ANQLTVVNTLQSDAVKEKFEAVMGEKANGFVSSVLSVVTNNNILSKADFNSVYTSAMKAAVLDLPVEPSLGMAHIVPYKGKAQFQIGYKGLIQLALRSGQVVGLNAGKVYEGQFKSFNALTEKLDIVDIYNPKKDEPIVGYFAYMKLSNGFEKTTYWTKEQVEEHGKKYSQSYDSKFSPWQTNFDAMARKTVLKSILSTYAPLTIEMQNANDFDNGKNTGIEPLEVKDVTPETDNESLLTDLLEDEPSVNTETGEIIEDTELDLDYGQINAK

SEQ ID NO: 397 - protein, 271 aa, Weissella koreensis
ANELVKQLKSEKVAAQFETTAGKNAAAFASEVAISVMGNKALENASLSSVVVEATKASALGLSLLPTVGEAYLVPYKGQAQFQLGYKGLVQLAMRSGQMKSFGTVKVYEGEHPRWDKYSQELHTDGDETGEVVGYYAQFTLINGFKKADYWTKSAVEEHRSRFSKSKSGPWSTDFDAMAQKTVLKSILQYAPKSSEMTRAMASEDMNGDISEGTAKPIDITPETETPKVEEANQNQQIDTNEMVDEIKEYAKETNEAPKEQTVSAADEFFK

SEQ ID NO: 398 - protein, 269 aa, Amycolatopsis sp.
TTQTVTSAVAQQDSSPAALVRKYRTDFATVLPSHIKPETWLRIATGALRRSSQLAHAAEKNPTSLLVALLDAARKGLEPGTEQYYLVPRKTKRGPEVLGITGYQGEVELMYRAGAVSSVKVEGVREHDTFAYNPGEHDRPVHEINWRANRGDLVLAYAYAYAKMRDGATSNVSVLSADDIAVILSKAEGADSPFSPWQWNPKAMWLKSAAHQLAKWVPTSAERVWQPDGPPLEAPPATPVTLPTVEDVVDAEVVEDWPTTPADTADSEQ

SEQ ID NO: 399 - protein, 269 aa, Leuconostoc pseudomesenteroides
ANEITLAKQLSSDKVVEQFAATAGESAKSFAKEVALTISGNPALQHAKLGTVIVEATKASALGLSLLPTVGEAYLVPYKGDAQFQLGYKGIVQLAMRSGQMKSFGAESVYEGENPKWDKYNQELVTDGEETGKIIGYYAFFTLVNGFKMAAYWPKEKVEAHRDRFSKSKKGPWSTDFDAMAKKTVLKSILQYAPKSSEMKRALAEDTQAEYVQAGIQDVTPEPANIEAPIETANAPEINAQEESLFGELSDVDKETAPNPFAQNLGGDN

SEQ ID NO: 400 - protein, 267 aa, Peptoniphilus sp.
TNIQKQENRALSPVNQMKNLLANQGMQNLFADALKENKDRFIASIIDLYNGDNYLQNCDPKEVAMEALKAATLNLPINKSLGYAYIVPFKNKGKLTPQFQIGYKGYIQMAQRSGQYKALNAGIMYEGMEIKRDFLRGTFEIVGEPKSDKAIGYFAYFQLLNGYEKALYMSKEDITDHAKRYSQSFGSDFSPWKNQFDEMAQKTVLRRLLTKYGVLTTEFQEAAKREEDEEVLKATEENAMIEMNSQEETIAVDPKTGEIIEETEAPF

SEQ ID NO: 401 - protein, 257 aa, Lactococcus fujiensis
KSAPVQARFQEVLGKKSSGFVSSLLTVVNNNNLLKRATPDSIMTAAMKAATLDLPIEPSLGFAYIIPYGQEAQFQIGYKGLIQLALRSGQITGLNSGIVYKSQFISYDPLFEELEIDFMQPEDEVVGYFASMKLSNGFMKVVYWTKARVENHKKRFSKAGAKSPWATDFDAMAQKTVLKAMISKFAPLSQEMQIAVIADNESETLEPKDVTPEQPLISIDEPKENENSQSQISIPEDQAPQQENEEFVEELFPVGQA

SEQ ID NO: 402 - protein, 269 aa, Actinoplanes italicus
PETIANAVAQRDQSPTALVADYRNDFAAVLPSHLPPATFVRLAQGVLRRDQNLMRTAMNNPGSLMTAMLDCARLGHEPGTPAYYFVPIKGAIEGWEGYRGVIERIYRAGAVQSVRAEVVRENDFYEYEEGMPHPIHRYERFASPEQRGPLLGVWAYAVMLDGGMSRPVEMGREEVLAHRDMNPSNNRSDSPWKKWERSMWLKCAVHELEKWVPSSTEYRREIARMSAPQPAAAAAPVTYVPPQVGQRDAIEGEVAEDWPEPAEVPGGAQ

SEQ ID NO: 403 - protein, 263 aa, Cellulosilyticum lentocellum; identical to SEQ ID NO: 190

SEQ ID NO: 404 - protein, 264 aa, Streptomyces sp.
SQIGNEIARQSHSPAAIIEQHKADLAVVAASHVRVDTFARLAVGVLRQNEKLAAAAANNPGSLMSALMTAARLGLEPGTEQFYLRPIKRKGQLEVQGIVGYQGIVELIYNAGAAQSVVVEVVRARDEFAWTPGALDEHRPPRWPGAMKQPHHKVDWFGDRGPLVGAYAYAVMQGGAISKVVVLNRDHIARAKAKSDGADTDYSPWRTDEEAMWLKTAARRLGKWVPTSAEKRTGVIERLDTPPAPLNEIDPDEDDEPIDGELVD

SEQ ID NO: 405 - protein, 267 aa, Clostridium kluyveri
PDKKMMVLSESHKALNKLLETKKEALPKDFNKSRFLQNCMTVLQDTKDIDKCQPISVARTMLKGAFLGLDFFNRECYAIPYNGNLQFQTDYKGEIKLAKKYSINPIKDIYAKVVRKGDEFQESIVNGHQTVNFKPLPFNNDEIIGAFAVCLFQDGSMIYETMTKQEIEDIRNNFSKAKNSPAWVKTPGEMYKKTVLRRLCKFIELDFNSIESKKTYDEASDFQFEHEPNKEVSNFDKGSIDEDKTVEADTETEAKEDNREYAFKESE

SEQ ID NO: 406 - protein, 258 aa, Paenibacillus popilliae
STSHLLTIHNNLEKLIDSKREAMPKSFNKTRFLQNCMTVLQDTKDVGKCDPQSVARTLLKGAFLGLDFFNKECYAITYGGSVQFQTDYKGEKKLAKKYSVRPVKDIYAKLVREGDEFIEEIKDGQPTVQFKPLPFNDSEIKGAFAVSLFEDDGLAYEVMSVAEIELTRKNYSKQPNGQAWVKSKGEMYKKTVLRRLCKNIELDFDTIEQAQAFEDSSDFEFNKEPKQAQQSPLNPQATVIDAEYEEVKEESDNETNQE

SEQ ID NO: 407 - protein, 249 aa, Clostridium ragsdalei
LDKQANGFITSLLNLKQDKLKGCNDMTVLGSALKAAPLKLPIDPNLGFAWIIPFKNHGKLEAQFQVGYRGFIQMAQRSAQYKKLNVTEIYEGQLKSFNPLTEELELDLDNKQSDEVVGYAAYFRRLNGFEKMVYWSKEKVTAHARRFSKSFGNGPWKTDFDAMARKTVLKNMLSTWGILSIDMQEAITSDSKIIKTTEDDYELLEEGTEDESNANVTDVEYTESDESGKEEDGKDPYEGTPFSENNTES

SEQ ID NO: 408 - protein, 269 aa, Paenibacillus polymyxa
PDKLLVIHDNLNKMLDEKSEAMPTSFNKTRFLQNCMAVLQDTKDIEQCDAKSVARTMLKGAFLGLDFFNKECYAIPYNENIGTKNKPRWIKSLQFQTDYKGEKKLAKKYSTRRVKDIYAKLVRDGDDFREEIESGQPTINFRPLPFNDGIIRGAFAVALFEDGGMIYETMSLKEIEKTRDDYSKQSTGKAWTKSPGEMYKKTVLRRLCKNIELDFDTIEQAQAFEDSSDFDMNKELKPQQQSPLNPNTTIIDAEYEEIKEEPADGPEQE

SEQ ID NO: 409 - protein, 267 aa, unidentified
  source 1..267: note = Candidatus Collierbacteria bacterium GW2011_GWB1_44_6
SNQIQIKSEVDLKMILANQYMKQINNFFGNEKQAMKFLSSVMSAVQRIPELLNCEPKSLINSFMTMAQLGLMPSEVSGEAYVLPYNNKNGKVAQFQLGYQGLVTLFFRAGGQKIRAEIVRKNDEVSYVNGEIKHTIDIFKSNEERGEAVGAYAVATINGQEVCKYMNATDILAFGSRFSKSWTTSFTPWKEANDPELNMWKKTVLKQLGKMLPKNESINLAIAEDNKDSIISDRLLPAVEESKNLTMGSIVKTEEPVIEVEPEEIKQ

SEQ ID NO: 410 - protein, 254 aa, Antribacter gilvus
SADVVIRQHATELTSVLPSHLAEKGDGWLNAAVAAVRKDRNLWNAANSDPGAVMNALAEAARLGLQPGSKEYYLTVRGGKVLGIVGYQGEIELMYRAGAVSSVIVEPVFERDGFEYTPGVDDRPKHRIDWDADDRGPIRLAYAYAVMKDGAVSKVVVVNKTRIRRAKDASATAGKSHSPWTSDEVAMWMKTAAHDLAKWVPTSAEYIREQLRAVKEVEAEPARASDPRPEPVHIVEAQILDEDPFPNAPEDGAA

SEQ ID NO: 411 - protein, 267 aa, Lactococcus sp.
SNQITKTQQTLKSPEVKAKFEEVLGKKADGFVASLLSVVGNSNLKTVEANSVMTAAMKAATLDLPIEPSLGFAYVIPYGREAQFQIGYKGFIQLALRSGQLTGLNCGIVYESQFVSYDPLFEELELDFTQQASGDAVGYFASMKLANGFKKVTYWSKEQVLAHKKKFVKSANGPWRDHFDAMAQKTVLKAMLTKYAPASIESKMIQTAITEDDSERFENAKDVTPDEPVISIDEPVTSEVSQNESSAESQEQFPEDEVEELFPIGKS

SEQ ID NO: 412 - protein, 256 aa, Nocardia puris
AESISSEVARQASPLAVVARYRSELAGSLPAAVRHDVDRWLMVAEMAVRRSPDLMEIVRRDQGASLMRALIECARLGHEPGSPEFYLIPRGGIVSGEESYRGIIKRILNSGEYQRVVARVVHERDRFSFDPRIDEIPDHRPAEGERGAPARAYAFAVRWDGTPSTVGEATPERIIAAKAKARGVDRKDSPWNSPTGVMYRKTAIRELASYVHTSAEPRPRPAAPTEPPAVDEVSTVYDAEVIDEVDVLDITAEPTA

SEQ ID NO: 413 - protein, 258 aa, Nicoletella semolina
LKNADPQSVFNAACMAATLNLPIQNGLGFAYIVPYQNKKEKKTEAQFQLGYKGLIQLAQRSGQFKRLVAVPVYEKQLIAEDPINGFEFDWKQKPENGEKPIGYYAYFKLLNDFTAELYMTTHEVDEHAQRYSQTYRTYLDKKSKGQWASSVWADNFEAMALKTVMKLLLSKQAPLSVEMQQAVLADQAVVKNVETNEFSYVDNQIEEAEYTELKVSTDIFEKCKQSILNKETTLQELCDSGYEFSQEQYAELEKLEVE

SEQ ID NO: 414 - protein, 255 aa, Paenibacillus macquariensis
SDKLLVIHKNLENLLDSKREAMPSNFNKTRFLQNCMTVLQDTRDIDKCDATSVARTMLKGAFLGLDFFNKECYAITYAGAVQFQTDYKGEKKLAKKYSVRPVRDIYAKLVKEGDDFKEEVKDGQQTIQFAPKPFNDGEVLGAFAVALFEDGGLVYEVMSKVDIETTRKNYSKQANGQAWTKSPGEMYKKTVLRRLCKNIELDFDTIEQAQAFEDSGDMDLNKEVKPPQVSPLNATVIDAEYTEIREGDPNATNQE

SEQ ID NO: 415 - protein, 272 aa, Anoxybacillus sp.
ATTQSLKNQIAKKQNSNIQQGVTLKQLLNSESMKKRFEEVLGKRAQQFATSILNLYNSEKMLQKCEPMSIISSAMVAASLDLPVDKNLGYMYIVPYGTTATPIMGYRGYIQLALRTGQYKHINVIEVYEGELQKWDRLTEEFEMDSKQKKSDVVVGYAAYFELINGFRKTVYWTREQIEAHRKKYSKSDFGWKNDFDAMAKKTVLKSLLSKWGILSIEMQNAFNEDEKEVDTKEVKDITSEVQEAEYIEAEAFEVPIETETPQQEEIVFDAQ

SEQ ID NO: 416 - protein, 268 aa, Ruminococcus sp.; identical to SEQ ID NO: 207

SEQ ID NO: 417 - protein, 266 aa, Peptoniphilus senegalensis
TNQIARKPVNEIKNVLSVPSVRNLFDNALADNAGAFVSSLIDLYGGDSYLQNCEPKDVVMEALKAATLKLPINKNLGFGYVVPFKNKNGKLVPTFIIGYKGLIQLAMRTGQYKAINSGIIYEGMEIKEDVLRGTLEIKGSKQSEKIKGYFAYFQLINGFEKALYMDVEEAADWGRKYSKSFAKGPWTTEFDAQAQKTCLRRLLSKYGVLSTEMQRLEKTEEDVDIAVGTIENNAVEELNIPSSQADYIVDEETGEILDDEEIVAPF

SEQ ID NO: 418 - protein, 257 aa, environmental Halophage eHP-30
TEQNQTPAKTESKSPIKAQLYKDNVQQRFQELLGERASAFMTSVMSVVKDNDQLSQAEPSSVLNAAMTAATLDMPIDNNLGMAYIVPYKDGKSGKTYAQFQLGYKGFIQLAQRSGQFKTISATPVRQGQIVTADPLRGYEFDFTQGQDKEVVGYAAYFALLNGFEKTLYMSKAEMEQHAASYAAGYKKGYSNWNRKFDEMALKTVIKQLLSKYAPLSVDMQKAQQTDQTVSVEEPNAIEQQEAAPEIDASSNNNQNQ

SEQ ID NO: 419 - protein, 272 aa, Streptococcus pseudoporcinus
ANQLTVVNTLQSDAVKEKFEAVMGEKANGFVSSVLSVVTNNNLLAKADFNSVYTSAMKAAVLDLPVEPSLGMAYIVPYKGKAQFQIGYKGLIQLAQRSGKVTKLNSGKIYKGQFKSYNALSEELDIDDIYTPKEDEEVVGYFGYMKLSNGFEKITYWTKERVEKHGKKYSQSYDSKFSPWQTNFDAMAEKTVLKSILSTYAPLTIEMQNANDFDNGKNTGIEPLEVKDVTPENDNESLLSDLLEDEPSVDAETGEIMENTELDLDYGSINAK

SEQ ID NO: 420 - protein, 264 aa, Hathewaya proteolytica
ADSKKELILKESYSVLDRLIETKISAMPKDFNRTRFLQNCMTVLQDTKDIEKCQPISVARTLLKGAFLGLDFFQKECYAIPYGGTLQFQTDYKGETKMAKKYSIRPIKDIYAKVVREDDLFEEEIKEGQQFVNFKPIPFSDKPIIGAFAVVLYQDGGMEYETMSKTQIEGIRDNFSKMKNGLMWTKTPEEAYKKTVLRRLTKKIEKDFDTIEQAKTYEETSDSEFKKEEKCNEKSVFDVEYSEVESEELEQQTMLENSPFGGEQ

SEQ ID NO: 421 - protein, 221 aa, Bacteroides pyogenes
QVADPQSVLNSAVIAATLDLPINPNLGFAAIVPYNDRKSGKCIAQFQLMYKGLVELCLRSGQFASLIDEVVYEGQIVKKNKFTGEYIFDEDAKTSNKVIGYMAYFRLVNGFEKTFYMTSEEVTAHAKAYSQSFKSGYGVWKDNFDIMARKTVLKLLLSKYAPKSIEMQRAITFDQAAVKGDLTETNVDEAEIEYIDNESGSDKIKQAAEDAVIQSQQKTLL

SEQ ID NO: 422 - protein, 268 aa, Eubacterium sp.
AERKQITTKEYLAEVKGGLENELNLNAKALPENFNQSRFVLNCISLIKSNLSNYNNITPESVYLALAKGAYLGLDFFNGECYAIPYSGEVNFQTDYKGEIKLAKTYSRNPIKDIYAKNVRDGDFFEEIIESGKQSVNFRPVPFSDKKIIGTFAVVLFKDGSMMYDTMSVKEIEEVRNNFSKAKNSKAWAATPGEMYKKTVLRRLCKLIDLDFNSQQRLAYEDAGDFDKEKADEPVADDTVNVFDAEFKEVEPENKDAAIIEEMGLEEA

SEQ ID NO: 423 - protein, 262 aa, Pediococcus pentosaceus
MNDISKVPMKVLVQQDKVQRMLENTLKGKTRQFTTSLINVVNSNQSLADVDQMSVIKSAMVAASLDLPIDQNLGFMWLVPYKGMATPQIGYKGYIQLALRTGQYKKLNTIVVHEGEMKYWNPLTEDFEYDPKGKESDEVIGYLGYLRMINGFEKTVYWTKQNIEDHRMKFSKMSGKAKPSGVWASNYDAMALKTVMRNLLSKWGIMSIEMQQAVVQDEKAPETDVRDVTPTETNSIDSLLAPEPKGEPINDSNEATVPTNAE

SEQ ID NO: 424 - protein, 270 aa, Bacteroides eggerthii
GTVTTVPQLKSMLANENVKSRFKEILGKKAPGFISSIVAVANSNTLLQKAEPQSIMNAAVIAATLDLPINPNLGFAYIIPYGNQASFQIGYKGMTQLAMRSGQYKTINVTEVYEGEIKSENRFTGEYTFGERKSDKIVGYMAYFSLTNGFEKYMYMSREECEKHGKKFSQTYKRGGGLWATDFDSMSKKTVLKMLISKYGILSIDMQRAQTFDQAVVKDDLVEKNIDEAEVSYEDNPTNADVRRNAMKEALEEAEVVDETTGEIFNQPAQ

SEQ ID NO: 425 - protein, 270 aa, Criibacterium bergeronii
EVNNMNNQMQQTATQVTPINQMKNLLANKGINQMFEQALKMNAGAFISSLIDLYNSDGYLQKCEPKDVAMEALKAATLNLPINKGLGFAYIVPYGKAPQFQIGYKGYIQLAMRTGQYKHINAGAVYEGEEVKENRLAGTVEILGDKKNDNETGYFAYFKLTNGFEKCLYMSKQEMTTHAQRFSKAFKNGPWQSDFSAMATKTVLRLLLSKYGVLSTQMQEAIAKENDDELQQQINQNANKEVIDIEKIDNKNVIDIEAIDAADDDIEAPF

SEQ ID NO: 426 - protein, 270 aa, Listeria sp.
ATNDELKNQLANKQNGGQVASAQSLDLKGLLEAPTMRKKFEKVLDKKAPQFLTSLLNLYNGDDYLQKTDPMTVVTSAMVAATLDLPIDKNLGYAWIVPYKGRAQFQLGYKGYIQLALRSGQYKSINVIEVREGELLKWNRLTEEIELDLDNNTSEKVVGYCGYFQLINGFEKTVYWTRKEIEAHKQKFSKSDFGWKKDYDAMAKKTVLRNMLSKWGILSIDMQTAVTEDEAEPRERKDVTEDESIPDIIDAPITPSDTLEAGSEVQGSMI

SEQ ID NO: 427 - protein, 271 aa, Clostridium thermopalmarium
ATVNELKNEIATKKETGVGSAGNTIKGLINSPAIKKRFEDVLNKKAPQYMSSIVNLVNGDTNLKKCDQMSVIASCMVAATLDLPIDKNLGYAWIVPYGNRAQFQLGYKGYVQLALRTGQYKAINVIEVHEGELIEWNPLTEELKIDFSQKKSDAIIGYAGYFELLNGFKKSTYWTKEQIIRHKNKFSKSDFGWKKDFDAMAKKTVLRNMLSKWGILSIEMQNAYTADQATIRPEAVETGDIKGNVDYVEADFEENYEGTPFEEVEEGGVNE

SEQ ID NO: 428 - protein, 269 aa, Virgibacillus chiguensis
ATNSSLKNQIANKGNGNQNTPQGYTVKQLMSASSVKNRFEETLGKKAPQFMASVINLVNGDTNLQKCDQMSVVSSAMVAAALDLPIDKNLGYAWVVPYGNKATFQMGYKGYIQLALRTGQYKNINVIEVYEGEVKSFNRLTEEIELEFEGKESDKVIGYVGYFELINGFRKTVYWSKDEIERHKKRFSKTGFAWKDNYDAMAKKTVIRNMLNKWGILSIDMQTAVTTDGNAVTQDFEQEDSGLVIDAEFSEVNEASEGQQEIKFENADA

SEQ ID NO: 429 - protein, 267 aa, Clostridium cochlearium
ATNESLKNQLATKKETGIGSAGNTIKSLINSPVIKKRFEEVLDKRAPQYMSSIVNLVNSDTNLKKCDQMSVIASCMVAATMDLPVDKNLGYAWIVPYGNKAQFQMGYKGYVQLALRTGQYKSINVIEVHEGELEEWNPLTEELKIDFSKKESDAVIGYAGYFELLNGFKKSTYWTKEQITKHKNKFSKSDFGWKKDFDAMAKKTVLRNMLSKWGILSIEMQNAYTADHGVIKNEIMETGEVKENVEYIEADFESYEGTSIEEGGSNE

SEQ ID NO: 430 - protein, 271 aa, Peptoniphilus grossensis
TNIQKQENRALSPVNQMKNLLKNQGMQNLFADALKENKDRFLASIIDLYNGDTSLQDCNPKEVVMEALKAATLNLPINKNLGYAYIVPYNSKGTTRPQFQIGYKGYIQMAQRSGQYKALNAGILYEGMEVKRDFLRGTFEIIGEPKSDKVMGYFAYFQLLNGYEKAIYMTKDEVTEHAERYSQSYGSKYSPWKKQFDEMGQKTVIKKLLSKYGVLTTEFQDAVKEEEDREVLRATENNAMLEMTNPDEEEETIEVNPETGEIIEDDVKAPF

SEQ ID NO: 431 - protein, 260 aa, Schleiferilactobacillus shenzhenensis
SAVSESKDLQHVDQLSVLNSAMTAASLNLPINQNLGFFYLVPYKGIAQAQMGYKGYIQLAQRSGQYQRLNAIPVYADEFGSWNPLTEELDYTPHFEDRKASDKPVGYVGFFKLANGFEKTVYWSRKQIEAHRDRFSKSSKSSASPWNTDFDAMALKTVLRNLITKWGPMTTDIQRANDADEGDYKNDLSTDTSEPKDVTPGASLEQFLGETDQQQKPATKPAPKKKAEEAKPNDLKPDVTHDPNEHTEQTSLSDDDLPFD

SEQ ID NO: 432 - protein, 261 aa, Gulosibacter molinativorax
TDLTEKIATKAVAVKKDPKIADLMKSYEPQFARSLGKSMDAAKFGQDALTAIKQTPKLLEADQRSLFGAIFLAAQLKLPVGGPLAQFHLTPRKVAGEMTVVPIIGYNGYIQLAMNTGLYSKVGAFTVHANDHFRTGANSERGEFYDYERATGDRGELTGVIGYAKVKGFDESSFVYLDAATVRERHRPKFWDKTPWASDEGEMFRKTAIRVLQKYLPKSIEAAPLALAAQADQATVRRVDGVDDLQIDHEDIAIAEVIEDD

SEQ ID NO: 433 - protein, 266 aa, Cereibacter johrii
TENTAQAPAAARQLTPIQAISQTLESDAFAPKISASLDGTGISPARFKRAALACLSRPEASYLVEKCDRGSIFTAVMNAAAAELELHPALGQAYIVPRGGQAVLQVGYKGFIALASRAGLAVEADVIYAGDRFSIRKGTNPDVSVEPELDPAKRGEWVAVYVITHYASGAKTLTFMTRAEVEAIRNRYSDAYKRGGAGAKTWNESPEEMAKKTCIRRASKLWPISVPGGGDDDGGEVIEADPAPVPAPRMRDVTPGGGLDRLAASL

SEQ ID NO: 434 - protein, 264 aa, Clostridium coskatii
SDKKMVVLNESHTMLNKLLETKQEALPKDFNKARFLQNCMTVLQDTKGIEQCQPITVARTMLKGAFLGLDFFNKECYAIPYKDNLQFQTDYKGEIKLAKKYSFNPIKDIYAKIVRQGDDFQEAIINGQQTINFTPVPFNNGEIIGAFAVCLFQDGSMLYETMAKQEIENTRKNFSKAPNSPAWTKTPGEMYKKTVLRRLCKLIELDFDSVECKKVYNETSDFEFENQQHEVSNFDKKDIDEDKIVEADVEVQDDNENNVPEDGE

SEQ ID NO: 435 - protein, 270 aa, Bacteroides stercoris
STITTIPQLKSMLANDNVKARFKEILGKKAPGFISSIVAVANSNTLLQKAEPQSIMNAAVVAATLDLPINPNLGFAYVVPYGNQAQFQMGWRGFVQLAMRSGQYKTINVNEIYEGEIKKSNRFTGEYEFGERASDKIVGYMAYFSLINGFEKFLYMSKEDCEKHGRKFSQTYKRGTGIWSTDFDSMAKKTVLKMLLSKFGILSIEMQRAQTFDQAIIKDNLAETDIDEAEVSYNDNPDNEEARRNAMKEALQEAEVVDENTGELFNTETK

SEQ ID NO: 436 - protein, 266 aa, Clostridium combesii
ANTKAIVLQETANNLNTLLKAKVKALPKGFNETRFLQNCMTVLQDTRNIEKCNSVSVARTMLKGAFLGLDFFSKECYAIPYNDYKTGKCHLEFQTDYKGERKLMKQYSVRPIKDIYAKVVREGDKFEEIIEKGIPTINFRPKPFSNEKIIGVFAVVLFEDGGLLYETMSVEDVEKIKVGFAKRDKEGNYSKAWTATPEEMYKKTVIRRLRKSVELEFDSVEQQKTYEEASEFDVKRDEEVKEEASPFENVDFEEAEEGNTIEAKQE

SEQ ID NO: 437 - protein, 262 aa, Oceanobacillus sp.
ATNDSLKNQLSSKQGNQNTPSGYTIKQLMGAESVKKRFEEMLDSKASQFMASVINLVNGDTNLQKCDQMSVVSSAMVAATLDLPIDRNLGYAWVIPYGNQATFQLGYKGYIQLALRSGQYRNINVIEVYEGELQSFNRLTEEIELDFEKRTSDKVIGYTGFFELINGFRKTVYWSKAEIEKHKNKFSKSGFGWKNDWDAMAKKTVVRNMLNKWGILSIDMQKAYVEETKDPSEPNGEVIDLNLTEDELTAAQEQFSDENANE

SEQ ID NO: 438 - protein, 270 aa, unidentified
  source 1..270: note = Candidatus Parcubacteria bacterium
TEYKRPQQPEQTKMLSAKLNQAGAPNKVSSFDVQLRDWFKKHSRKMQTLAGSKEEANKIITSLIFVAQRNPKLMTCTMESIGECLMQSAQLKLYPGPLQECAYVPFGNRATFMPQYQGLCKLAYNSGVVRSIATEVVYANDLFEFELGTNAYLRHVPTLSDNRGERIAAWCVVKTTHGEVIIVKPISFIEGIRKRSPAGNKKDSPWNTSDDDYDAMARKTVLKQALKTIPKSSDLAAAIQVDNAVESGSVDNVVTHIEPVTDPTPEETKE

SEQ ID NO: 439 - protein, 267 aa, Lactococcus sp.
ANLTPTQTVLKSDAAKRKFEEVLGKKTNGFVGSLLSLVGSTNLKNVDSNSVMTAAMKAATLDLPIEPSLGFAYVIPYGREAQFQIGYKGLIQLAIRSGQVTKLNAGPVYENQFIKYDSLFEELEIDFSMPQGVEIAGYFASMELANGFRKIIYWDKEKVTAHGKRFSKSFNRSSSPWQTDFDAMATKTVLKAMLSTYAPLSTEMQQAIVADNESATPKDATPVTDDLVLEAVEDSKQIEENEIINDQVASENYQEPQGEPEVLDLEL

SEQ ID NO: 440 - protein, 263 aa, Nocardia ninae
AESISKEVARQANPLAVVAKYQNELGKSMPAAIRGDVGRWMMVAEMAVRKNPKLLSIVQADQGASLMRALIECARLGHEPGTKYFYLVPRGNQISGEEGYHGIIKRVLNSGHYQKVLARTVFERDEYSFDPLTDQLPTHVPASGERGKPVSAYAFALHWDGTPSTVAEASPERIAAAKAKSYGTDRKDSPWQSVTGVMYRKTAIRELEPYVHTSAEPQPRQDNAGSRGAVMDPSTYDDAEPLDADVLDITADQIAEHDGEGAL

SEQ ID NO: 441 - protein, 270 aa, Clostridium cadaveris; identical to SEQ ID NO: 241

SEQ ID NO: 442 - protein, 265 aa, Virgibacillus sp.
ATNDSVKNQIANKNQGSNQVNPNNLGLKQLLSTPTMRKKFDEVLDKKAPQFMSSLLNLYSNDSYLQKAEPMSVVTSALVAATLDLPIDKNLGYAWIVPYGGKAQFQLGYKGYIQLALRTGQYRNINVIEVYEGELKSFNRLTEEMELDFEQKQSDKVIGYTGYFELINGFRKTVYWSKEEIEKHKKRFSKSDFGWKKDWDAMAKKTVIRNMLNKWGILSIDMQKGIVEDNKDPIEKANEFDEQDIIEADFSEVNDDQEIDFSDAQ

SEQ ID NO: 443 - protein, 268 aa, Clostridium liquoris
TTASELKNQLATRKETGVGSAGNTVKGLLESPAIKKRFEEVLKQRAPQYMSSIVNLVNGDANLKKCDQMSVIASCMVAATLDLPVDKNLGYAWVVPYGNKAQFQLGYKGYVQLALRTGQYKSINVIEIHEGELIEWNPLTEELRIDFEKKKSDAIIGYAGYFELINGFRKSTYWTKEQITKHKNKFSKSDFGWKKDFDAMAKKTVLRNMLSKWGILSIEMQNAYTADQETIKSEVLETGNIKENVEYVEADFDVDFEGTPFEEGVTNE

SEQ ID NO: 444 - protein, 259 aa, Paenibacillus pinihumi
ADANKLLVINEKLIKLIESKQDAMPKSFNKTRFIQNCMAVLQDTDEIDKCDATSVARTLLKGAFLGLDFMNKECYPIIYGGKCTFQTDYKGEIKLAQKYSVRPVLNIYAELVREGDFFLKEVKDGQRTIQHKPPEGFNDGKVIGAFAIVLYKDGGMDCESMSVAEIETTRKNYSKQANGPSWTKSPGEMQKKTVLRRLCKTIQLDFDTIEAKEAFEDGGDFDFKQDPKPQQQSPFDKNATVVDAEYEEVEEEDQSESAT

SEQ ID NO: 445 - protein, 267 aa, Oceanobacillus sp.
ATNSTLKNQISNKKQGNNQVGKTQGTTMKQLLASPAVMNRFEEVLGKRANQFTASILGLYNSEKMLQKAEPMSVISSAMIAATLDLPVDKNLGYAWIVPYGGKAQFQMGYKGYIQLALRTGQYRNINVIEVYEGELKKWDRLTEEIELDFESRTSDKVIGYTGYFELINGFRKTVYWSKEDVEKHKKRFSKSDFGWKNDWDAMARKTVIRNMLNKWGILSIDMQKGMVEDSKDPVEVNEEFSSDVIDADYEVVGENEQQDFTVEENA

SEQ ID NO: 446 - protein, 242 aa, Nocardia terpenica; identical to SEQ ID NO: 205

SEQ ID NO: 447 - protein, 260 aa, Paraliobacillus ryukyuensis
ATNDTLKNQISNKKNNQVAEGKQGTTMKGLLNSPAVMKRFEEVLGKRANQFTASILSLYNNEKTLQKSEPMSVISSAMIAATLDLPIDKNLGYAWIVPYGNKAQFQLGYKGYIQLALRTGQYRNINVIEVYEGELVKWNRLTEELELDFEQKKSDKVIGYTGYFELINGFRKTVYWSKADIEKHKQKFSKSNFGWSNDWDAMAKKTVIRNMLNKWGILSIDMQKAYSTDEIEQEQESNDFIDGEWAEVSEDDITEAMNEV

SEQ ID NO: 448 - protein, 204 aa, Dorea sp.
AVNNSLAKRDQSMKLSVYLQNDAVKKQINQVVGGKNGTRFISSIVSAVQSTPALQECTSPSIVNAALLGEALNLSPSPQLGQFYMVPFDNRKKGCKEAQFQLGYKGYIQLAERSGYYKKLNVLAIKEGELIRYDPLDEEIEVELIDDDVIREETPAMGYYAMFEYENGFCIQQKWRSEDFGTFRAGQNSGKGSLEVFFFLVQRF

SEQ ID NO: 449 - protein, 270 aa, Lactiplantibacillus plantarum
SNELAHMPMKQLVKQDAIQQMLSRTLADKASQFSTSLINLVNGNQSLAKVDQMSVIQSAMVAATLNLPIDQNLGYMWLVPYKGRATPQIGYKGYIQLAQRTGQYLAMNAIAIHSGELKGWNPLTEDFQFDPMGRTSDEVIGYVGYFKLTNGFEKTVFWTKASMEEHRMSFSKMSGGKTPQGVWASNYDAMAIKTVLRNMLSKWGPMSIEMEQALANDETAPQTPLNVEAEESASETTDNMLDKFRQQQGEVNTSDQEHNTEDQGDPRDQS

SEQ ID NO: 450 - protein, 259 aa, Leucobacter luti
SDLSQAAVAVKKSKTVEDYLTEYEPQFQRALGKSMDAAKFSQDALTAIKQTPQLGQADLQTLFGSLFLAAQLKLPVGGPLAQFHLTPRKRGDKLEVLPIIGFGGYVQLIMNTGLYSKVGAFLIYEKDYFDEGANSERGEFYDFKKSRGDRGPVVGVIAYVKLKGFDESQYVFLDADTIRSRHRPRYWEKTPWGSDEGEMFKKTGVRVLQKLLPKSVEAAPLALAADADQATVRKVDGIEDLTIQHDVVDAEVVPDGVPV

SEQ ID NO: 451 - protein, 261 aa, Paenibacillus donghaensis
SNTQLATIHNNLERLIDSKRDAMPSSFNKTRFLQNCMTVLQDTYGIEKADPVSIARTMLKGAFLGLDFFNKECYAIIYGGKVEFMTDYKGEVKLAKKYSIKRIKDIYAKVVRAGDEFEETIEGGNQSINFKPLPFNDGEVLGAFAVVVYEDGSMNYDTMSVKEIESIKENFSKKSKDTGQFSKAWVVTTSEMYRKTVLRRLCKNIELDFDTIEAKQAFEDGGDFEFNKDKKPAQESPLNPKSTVIDGEFTAVGEGAADGTE

SEQ ID NO: 452 - protein, 270 aa, Capnocytophaga sp.
ETQVLQKQSLANFLNKSDKFLEQNLGAKKSEFVSNLLALSDSNKELSQCEPADLMKCAMNATALNLPLNKNLGYAYVIPYFDGKTNRTIPQFQMGYKGFVQLAIRSGQYKTINTCEIREGEIKRNKVTGHIDFLGENPSGAVIGYLAYIELLNGFQQSLFMTIEEVQAHARKYSKIYAKTNRGLWKDEFDLMAKKTVLKLLLNRYGVLSVEMQKAIEKDQADNEGNYIDNPQGRYIQDAEVIEQNEPTENAQPVQPVTSEEPNKVDFKDV

SEQ ID NO: 453 - protein, 270 aa, Paenibacillus sp.
AKALLENKLQERAAGASTPSTQGTSLKALLNSPAIKKRFDELLDKRSAQYMTSIVNLYNSDAMLQKAEPMSVISSCIVAATLDLPVDKNLGYAWIVPYSGKAQFQLGYKGYIQLALRTGQYKAINVIEVYEGELVKWNPLTEALELDFEKRKSDAVIGYAGYFELINGFRKSVYWTREQIESHRKKFSKSDFGWKKDYDAMAKKTIIRNMLSKWGILSIEMQDAYSKEIEAIPPLNNENEEDPPIDLTPEDYRVGDEPQDGKEQGEMNFE

SEQ ID NO: 454 - protein, 267 aa, Chitinophaga lutea
SNVNAPAAPVKSKIEVLKDIMNAPSVQEQFQNALRENSGVFVASVIDLFNSDTYLQNCEPKQVVMECLKAATLKLPINKNLGFAYVVPYKSNGKQIPQFQIGYKGYIQLAMRTGQYRIINADKVYEGEYRTKNKLTGEFDLSGTATSETVVGYFAHIEMLNGFAKTLYMTKEKVAAHAKKYSKSFGKETSPWHTEFDAMALKTVLRNLLSHYGYLSVEMMGAMNADIESDQVGSEVSQTINDKANKQEMTFDDAEVVDDDEKEQNPI

SEQ ID NO: 455 - protein, 271 aa, Priestia abyssalis
ATNQSLKNQLQSRQSAGTPAQQSNSLKALLSSPTVKKRFEEVLDKRSAQFMTSIVNLYNSEKMLQKCEPMSVISSAMVAATLDLPVDKNLGYAWIVPYKNTASFQLGYKGYIQLALRTSQYRFINVTPVHEGELMKWNPLTEEIEIDFDARQSDVIIGYAAYFELLNGFRKTVYWTKNQVEKHRKKFAKSDFGWKNDYDAMAMKTVLKAMLSKWGILSIEMQKAYSEDEEPRELKDITEEAQEVDYIEAEVIDVPAEEKASAFDQENFHIE

SEQ ID NO: 456 - protein, 260 aa, Leifsonia xyli
AVKKNPTIEDYLIKYEPEFQRALGASMDAAKFAQDALTAIKQNPKIGHSDPRSLFGALFLAAQLKLPVGGPLAQFHLTTRTVKGNLTVVPIVGYGGYVQLIMNTGLYSRVSAFLIHAGDYFVTGANSERGEFYDFRRADSDRGEVKGVIAYAKVKGHNESSWVYIDAETMRAKHRPKYWESTPWADDAGEMFKKTGIRVLQKYLPKSVESLNVALAASADQAIVRKVDGVPDLDIQHDRDTETVAVPEQPVSVPQPGDET

SEQ ID NO: 457 - protein, 261 aa, Clostridium thermobutyricum
QSTGDIVFPQNYNYSNALKSAQLILAETVDRNKVPVLQSCSKPSICNALLDMVIQGLSPAKKQCYFVPYGGKLQLMKSYLGNIAATKRLKGVKDVFANVIYEGDVFEYKLNLNTGLIEIEKHEQKFENISKKILGAYAVVVRENQNNYVEVMNIEQIKNAWNQGAAKGNSQAHKNFAEEMAKKTVINRACKRFVNTSDDSDTLIESINRTNEYKEEDIIETTKSEVGEEIKENANTENLGLEDTEVVEAEVIENIEFEGDK

SEQ ID NO: 458 - protein, 249 aa, Clostridium jeddahense
LGERTPQFISSIVSLVNADANLQRAFYDAPVTVIQSALKVATFNLPIDPNLGYAYIVPFNNTVKNPDGSIRKRIEASFIMGYKGMNQLALRTGVYKTINVVDVREGELKSYNRLTEDIELDFVEDDEEREKLPIIGWVGYYRLINGTEKTIYMTRKQIETHEKKNHKGQYMGKGWREDFDSMAMETVFRRLIGKWCLMSIDYQRANPGTLAAADALAHGQFDDEDPLPDAVPLQAEAQEVNPETGEVQS

SEQ ID NO: 459 - protein, 269 aa, Anaerovirgula multivorans
DAKHLTVVHQNLNTLLKAKADALPKGFNQTRFLQNCMTVLQDTKDIESVEPKSVARTMLKGAFLGLDFFNKECYAIVYNKKAGNSWIKTLEFQTDYKGEIKLAKKYSINTIKDIYAKLVREGDEFEEGVKDGKQVINFKPKPFNNNKILGAFAVAYYENGSMIYDTMSVEEIESVKKAYAKADKEGKYSKAWIESTGEMYKKTVLRRLCKLIELDFDTIEQKQAFDEGSGMEFKQEGKTDKPKSSLEAEFVEAEYEEVEESETSEVVEE

SEQ ID NO: 460 - protein, 241 aa, Nocardia pseudobrasiliensis
SSIANAANASELTPASIVNRYRDDIAAVLPPKLQARIDRWLRLAIGAVNSNADLVDRVRADQGASMMQTLMKCAALGHEPGSGLFHLVPKGPRIEGWEDYKGVLQRIDRSGVYARVVVEVVHANDDYAYDPNLDDRPQHKRAAADRGEPVSAYAYAVYPNGAVTAVAEATPELIAASKAKARGADNASSPWRAPGAPMHRKVAIRQLEKFVATSAEDMREVAVRNAAPDVEDAPADYYQEP

SEQ ID NO: 461 - protein, 260 aa, Paenibacillus rhizosphaerae
SSKLVEINSKLDSFLDAQHKAMPKGFNKTRFLQNSMSVLRDIEGLEQCDPKSVALVMLKGAFLGLDFFNKECYPVVYAGKVEFQTDYKGEVKLVKKYSTKPVREIYAKLVRQGDDFSEEIVAGSQTINFKPLPFNNGEIVGAFAVVNYVDGTMQYDTMSTEEIEKIKVNFSRKSKKTNEYSKAWVVTPGEMYKKTVLRRLCKTIDLDFDTIEQAQAFEDAADMDFNQDSKPQQQSPLNPMVIDVEYEEVKEEQADAAEQE

SEQ ID NO: 462 - protein, 262 aa, Brevibacillus porter
ADQNKLVVIYNNLEKLLDSKREAMPTSFNKTRFLQNCMTVLQETKDIELCNPTTVARTMLKGAFLGLDFFNKECYAVVYKGSVEFQTDYKGEVKLAKKYSTKPVREVYAKLVREGDEFAEEINSGNQTINFRPKPFNNEEILGAFAVVNYMDGTMAYDIMSKEEIEKIKENFSRKSKQTGEYSKAWVVTPGEMYKKTVLRRLCKNIDLDFDTIEQRQAFEDAGDVDFNQEVKPAQQSPLNSTVIEAEFEEVSEEQTNAAEQE

SEQ ID NO: 463 - protein, 261 aa, Parageobacillus thermoglucosidasius
AKQADLKNKLANKNSTNPTAYLKNLVYAPTVQQKFKEVLKEKAAHFLTSLISLVDSSPDLQKCNPMTIIASAMKAATLELPIDKNLGYAWIVPYKNVATFQIGYKGYIQLALRTGLYRSINVIEVYEGELRKWNRLTEELDIDEGARKSDHVIGYAGYFELTNGFIKRVYWSKEDIERHRKKFAKSDFGWENNYDAMAKKTVLRNMLSKWGILSIDMQRAYVNDIDDPEQTKEVIDVEWSEIIEEANVANSPEQQEIVFEQ

SEQ ID NO: 464 - protein, 260 aa, Pseudoalteromonas sp.
SLSLQEYQNLLYGKLTACKGQFDACLSENGYKLDFNTELNYVYQIVMSGLNVEYSFPYTPVESVITSFLKAAKIGLSLCPTEQLCFLKTEYSESSGQYVTQLGLGYKGILKLAYRSGKVKQINANVFYEKDNFQYNGVNSKVTHTTTVLSKAMRGQLAGGYCQTELIDGSFKTTVMPPEEILAIEEQGKAMGNEAWLSVHVDQMREKTLIKRHWKTLCPCIYRDSVMNDPMLFDDQDCQHSSNQQAYEEQFESAYSREAY

SEQ ID NO: 465 - protein, 159 aa, unidentified
  source 1..159: note = Peptostreptococcaceae bacterium VA2
QPFLVQRYPHLDVVLNDQVHVLKSFFFQNHIILYLYKYIECLQIFHKPLLKGDRGKVIGYYAVYHLEPNGYNFVFMTYDEVKNHGKKYSKNFEGGIWEKEFDSMAKKTVIKKLLKYAPLSIEMQKAVTFDESVKGSIDNDMLLVESIEDVEEIQLDTNI

SEQ ID NO: 466 - protein, 262 aa, Dysgonomonas capnocytophagoides
STQQVQQQTKPLSLANFLNAPSTANFLKETLAEKKSEFVSNLIALCDADPKLAQCDPAQLMKCAMNATSLNLPLNKNLGYAYVIAYKGVPSFQIGYKGLIQLAIRTGQYKFINATEIREGEIRHNKITGEVIFNGEKPDAPIVGYMSYLELVNGFTASLYMTEEQIEQHALRFSQTYKNDKQYRSSTSKWSDPLARPTMCKKTVLKLLLGTYGLMTTEFAKALDSDSDDEVSTSGHRFEEAEIVQQGEPNEEQSDEPKRMEI

SEQ ID NO: 467 - protein, 231 aa, Nocardia aurea
SESISAAADAQKVTPRIVLDRHRDAFAQVLPPTINLDRWLRLAESAINASAGLLDIFRRDRGASALKALMKCAQLGHEPGSGLFHLVPKGQAIEGWEDYKGILQRILRSALYAKVVVAPVYANDEYAFDVNVDERPRHKQAAGDRGEPVRAYAYAVHRDGSTSTIAEATPAMIAGAKAKGHKTDASTSPWQNPRAPMHQKVAVRELERFVSTSAVDLRVTGDVTDLIIEEP

SEQ ID NO: 468 - protein, 252 aa, Paenibacillus sp.
TMDYVTKIQDALDRELDAKHDALPSGFKKTRFSENCRAYVKDYKDLQKYDAEEVASVLFKGAVLGLDFLAKECHVITEGSALRFQTDYKGEMTLVKKYSVRPILDIYAKNVREGDDFREEISGGKPLIHFNPRAFNNSKITGSFAVALFTDGGMVYETMPAEEIESIREHYGKNPGSDTWEKSQGEMYKRTVLRRLCKTIEIDFDAEQSLAYEAGSSFEFDREQQPKKRSPFNPPEVEESEVLSNDGITETQ

SEQ ID NO: 469 - protein, 269 aa, Lactobacillus sp.
NSLSGALNSRNQAGSPTSMIKNLMRSDSIKNRFDEVMGAKAPQFMASITNLVNSNQDLQHVDAMSVVASAMVAATLDLPIDPNLGYMYIVPYRGQAQPQMGYKGYIQLALRTGQYKHINALPVYDDEVKSWNPLTEELEYESSGTSHDNQTPAGYVGYFQLINGFEKTTYWTYDQINSHRQKFSKMSSKTDPTGVWKSNFDAMALKTVLRNLISKWGIMSIEMQQAFVKDERPQEFDHETGEIQDVQEVEAEEENVAPETQGSTDKKEE

SEQ ID NO: 470 - protein, 255 aa, Clostridium diolis
ATNSSLKNQLIEKEQSTVNVQETIFKNLINSDEIKSKFTEVLKDKAFEYINSIINLVKEIPVPNALGASDSHQSADLGSLLIECEPRSIIDACMIAASLDLSIDKNLEYVWIIPYKKKSNFQLGYKGYIQLLLRTGEYKAINVIEVYEGQLKSWNPLTEEFDIDVSAKKSDAVIGYAGYFEMVNGFRKYVYWSKDNMDAFRNNSFKGDPRWNNDYKAMAKRTVMRNMLSKWGRLSAEMQRAYLEDINTDKFINGN

SEQ ID NO: 471 - protein, 237 aa, Clostridium saccharobutylicum
ATNSSLKNQLIEKEQTTVNVQETMFKNLINSDDVKSKFTEVLKDKAIQYINSIINLVNSDKDLIECEPKSIIDACMSAVSLDLSVDKNLEYVEIIPYKKKANFQLGYRGYIQLLLRTGEYKSVNIIEVYEGQLKSWNQLAEEFDIDFTYKKSDAVIGYAGYFEMLNGFRKSVYWSKENMDALRENSFKSDTRWNNDYKAMAKRAVIRNMISKWGSLSIEMEKAYCEDLNTDKFVNGN

SEQ ID NO: 472 - protein, 260 aa, Paenibacillus sp.
AANTQLITIHNNLEKLIEAKKDAMPQGFNKTRFIQNCMTVLQDTYGIEKCEPTTVARTLLKGAFLGLDFFNKECYAIPYGASMNFQTDYKGERKLAKKYSVRKVKDIYAKLVRAGDVFEENITDGQQTIQFAPVPFNNGDIVGAFAVVLFHDGGMLYETMSIAEMEHIKENYSKKSKDTGKFSKAWEVSTGEMYKKTVLRRLCKNIELDFDTIEQARAFEDAADVDFNKKTAPQQTSPLNVVEAEYEVVNDGSATEAQSE

SEQ ID NO: 473 - protein, 268 aa, unidentified
  source 1..268: note = Desulfobacteraceae bacterium
ATPNTPTTTDAGDFLKKSEKSLKNYAVRKYDFTSFLKSAMIAINDNTTLSECLRTEAGKKSLFNAMRYAATTGLSLNPQEGKAALIGYKNKAGEMVLNYQIMKNGLIDLALSSGKVEFVTADLVRANDEFSIKKSASGDDYSFSPAIRDRGEVIGFVAALKLKGSATYVKWMSTEEVAEFRDKYSSMYKNRPDASPWTHSFNGMGIKTVMKALLRSVSISPDVDAAVKSDDYIEAEFTVHGTTADDAVTQLQTPSKPVKAEEGQGELL

SEQ ID NO: 474 - protein, 179 aa, Clostridium akagii
ATSESLKNQLVNKETRPPKDPFKALVYSAGIKKRFEDMLDKQANGFITSLLNLKQDKLKSCDDFTVLGSALKAAALKLPIDPNLGFAWIIPFKNHGKLEAQFQIGYKGFIQMAQRSGQYKKLNVTEIYEGQLKSFNPLTEEIVLDLDNIKSDLRKINKRYLIVMRMNLLALHLRKISKG

SEQ ID NO: 475 - protein, 231 aa, Paenibacillus gorillae
LEAKHDALPSGFNAVRFVQNCKAYLPEVRNFERFNPDEIALQFLKGAILGLDFLAKECHVITEGSAARFQTDYKGEMKLAMKHSVRPLLNIYAKNVREGDVFRESVVEGRPVVSFDPLPFNNSKIIGSFAVAQFNDGGMDYESMSSTEIESIRTHYGKNPGSDTWEKSQGEMYKRTALRRLCKTIEIDFDAEQRLAFDAGSSFEFNREPRPQQQSPLNLESEVLTDEVEQG

SEQ ID NO: 476 - protein, 165 aa, Acetobacterium dehalogenans
CLRSWTRSFSNSVPLKIRFRLLYTAFLSQGSPLSSVNTTQSADKGSPIFFYAMFKTKDGGYGFEVMSVEDVRAHAKKYSQSFSSAYSPWSKNFEEMAKKTVLKKALKYAPLKSDFVRGIVVDETIKREISEDMYAAPSIEIEYEVDEDGVIQDEPTSNELTEAEK

SEQ ID NO: 477 - protein, 261 aa, unidentified
  source 1..261: note = uncultured organism
SNELQNIKPEVFGEVEDKLGSLADNNGIDLPENYSARNALKQAYLKLQSKDEPVFDKYKDETIYNALLDTLTQGLNPGKDQVYYIGYGNHLTAQKSYFGNIALAKRMAGVQEVSSNVILEGDEVDISIERGQQVIESHDRNFDSMDGQVKGAYAVISFEDERKDKYEIMTLKELKQAWAQGKSFGGNGKSPHHKFTKEMAKKTVINRALKPLIKASDDSGLIKEKPKLEKLKDGQQERTEGEKIEEVDVDKEEVVEVDYDV

SEQ ID NO: 478 - protein, 252 aa, Paenibacillus sp.
TVAIELQVQETLDRILDSKHDALPSDFNKKRFSENCKAYVADEKDLHKYSPEEIAANLFKGAVLGLDFLAKECHLISGGVELKFQTDYKGEMKLTKKYSVRPLLDVYAKNVREGDEFREEVIEGRPVIHFAPLPFNASSIIGSFAVALFQDGGMVYESIPAGEIEEIRKNYGKSLGDAWDKSQGEMYKRTVLRRLCKTIETDFDAEQRLIYDAGGAFEFTKQPARSRQQSPFNPPEESEVTQDDRVAETDQG

SEQ ID NO: 479 - protein, 242 aa, Paenibacillus sp.
ATEQIISSLEALLEAKHDALPSGFNPTRFVQNCIAYLPEIRNWDRFNAEDLAIQFFKGAVLGLDFLAKECHIIAEGSGVRFQTDYKGEMKLAMKHSVRPLLTIYAKNVREGDCIEEAVIEGRPVINFNPLPFNNSSISGSFAVAQYTDGGMVYETMSAEEIEAVRTNYGKNPGSDTWDKSKGEMYKRTVLRRLCKTIEIDFDAEQRLAFEAGSEFDFSKQPRPQQRSPFEEKEVGPDEVEQG

SEQ ID NO: 480 - protein, 252 aa, Paenibacillus graminis
TVAIETQVQETLDRILDSKHDALPSDFKKKRFSENCKVYVAEEKDLHKYTIDDIVANLFKGAVLGLDFLAKECHLITGGVDLKFQTDYKGEMKLTKKYSVRPMLDVYAKNVREGDIFREQIIEGRPAIHFDPLPFNASKIIGSFAIALFQDGGMVYESIPAGEIEEIRKNYGKSLGDAWEKSQGEMYKRTVLRRLCKTIETDFDAEQRLIYEMGGAFEFTKQPTRSRQQSPFNPPEESEVIQNDRAAETDQG

SEQ ID NO: 481 - protein, 163 aa, unidentified
  source 1..163: note = Gallionellaceae bacterium CG1_02_56_997
GRKEVERIRDGSRGYQAAKKYKKESTWDTDFVAMGLKTAIRRICKFLPKSPELATALAMDEQAGRQNLNLDDVINGSYTPVVDKDTGEIVDVADGGKTGNSNAATSTKLEKLEKIVAALRDANSVEALDEIYIRAEGDLDDANLEIAMREYRKCKDAISNSLI

SEQ ID NO: 482 - protein, 169 aa, Pedobacter nototheniae
STEQSQQQTAARVPAKFQEGTVDSILKRVSDFQNTGELVLPANYIPENAVRAAWLMLMETTDRNDKPAIEVCTKESIANAFLEMVTKGLSVVKKQCYFVVYGNKLSLEDSYIGKIAIAKREAGVKEVNAVTIYEGDIFKYENDIETGRKRILEHKQELKNINPDKIVGA

SEQ ID NO: 483 - protein, 241 aa, Ferrimonas kyonanensis
NQMINEPDFVTALKDSRETYIDLTQNGGFNLNYGLEAGWAHQQIEASRYQNLDLTCSEPGSIMQAFCEAARLGLSFDPRKKHIYLMGQKDVQSGRTITILYVGYKGMIALACRTGFMIGGHADLVFEEDTFTYRSGTQLPVHEHDGRPNHERGRLKCGYVVAHQPGGMVKTLLVPKEVLLEAASNGLNAGGSNNTWCGPYMEMMYQKTCWRYAFNAWYSELEAVGMTQAQLESATTAVSYQ

SEQ ID NO: 484 - protein, 243 aa, Pseudoalteromonas spongiae
NKFQHLQTELSSQLLSTKERFNELNNKNNLKVNFEEEYNFFYHLVTSSFYNINGIATCTFSSLKEAFLNIAKYGLSINPKLNLCYIRTEQSCAQANVNIAVYDFGYKGLLKLITRTGKVKIVTADVFYENDNFEFRGTREPVKHSTKTLSAAARGAMAGGYCSSELVAGGVVTTIMTPEELREIESICQSTGNEAWNSVFIDELRRKTLIKRHWKTLMQVIEEQNLSVPIEETYQCDFANGGY

SEQ ID NO: 485 - protein, 180 aa, Companilactobacillus kedongensis
MKDLARIPVKELVRSDTIKSKFNDVLGKRAPQFISSIVNIVNSNQDLKNVDQTSVISSALVAASLDLPINQSFGYMYLVPYSGKAQPQMGYKGYIQLAQRSGQYKRLNAISVSKEKVPDKMVIFIPDYRMEEAETQIDMYQDHIEDVKAGRVEPTRCGKCDYCKSTAKLGKIVSMDDLID

SEQ ID NO: 486 - protein, 262 aa, Peptostreptococcus anaerobius
SNQVTESKKGYVAEKNITDSALNAINKYMNDGVLHLPKTYSVENAMKSAYLTLSQAKDKNGKSVLESCTKESIYQSLLDMAVQGLTPAKNQCYFIPYGSKLTMSRSYLGTIAVTKSAVPEVKDVKGYAIYDKDVFETEFDYNTGCIKIKKFERNFDSIDTNSIKGAFALIIGEHGVLHTEVMNMAQIRNAWSMGATNGKSKAHNQFTDQMAIRTVINRACKFYINTSDDTSVLFADSYANSDEDTSSEREVEIVDENVREKK

SEQ ID NO: 487 - protein, 237 aa, Shewanella morhuae
QTAQVKLSVPHQQVYQDNFNYLSSQVVGHLVDLNEEIGYLNQIVFNSLSTASPLDVAAPWSVYGLLLNVCRLGLSLNPEKKLAYVMPSWSETGEIIMKLYPGYRGEIAIASNFNVIKNANAVLVYENDHFRIQAATGEIEHFVTSLSIDPRVRGACSGGYCRSVLMDNTIQISYLSIEEMNAIAQNQIEANMGNTPWNSIWRTEMNRVALYRRAAKDWRQLIKATPEIQSALSDTEY

SEQ ID NO: 488 - protein, 241 aa, Helicobacter trogontum
SNITTIQRKNEALALLENKEIQERLCALCGNEASKDKFKASLLNIALDSNLSACSMQSIVKASLDIAGLKLSLNKNLGKAYIVPRKVKIGNDYITEARIDIGYKGWLELAKRSKLSVKAHSVFDCDDFVYSVDGVDEYMKLTPNFELRQEHDSAWVKEHLKGIVVGIKDLKSGDSEVKFVSKGTLLKIMQKNDSVKNGKYSAYTDWLHEMLLAKAIKSCLSKTAMSEDTFYLIISNNKLFI

SEQ ID NO: 489 - protein, 258 aa, Pseudoalteromonas sp.
SISQQDYENLLYSKLYECESQYQAYLAEHNEKLNFNAELNYMYKAVMSGVGIEGGFPYTPLESIVESFLKAAKLGLSLDPSEQFCFLRSQYDHSTGLYHTELGLGYKGVLHLAYRSGKVKQIVSNVFYNKDNFQFNGPNSKVTHTMTVLSTSARGNLAGGYCQTELVDGSFIVTVMPPEEILAIEEQGKSVGNPAWLSAHVNQMREKTLILRHWKTLYPAIYSSSLLDSAQIFDDECEEFPFSSPSQGFSESQTIGSY

SEQ ID NO: 490 - protein, 237 aa, Shewanella xiamenensis
QTAQVKLSVPHQQVFQDNFNYLSSQIVGHQVDLNEEIGYLNQIVFNSLATTSPLDVAASWSVYRLLLNVCRLGLSLDPEKKLAYVIPSLSETGEKIMKLYPGYRGEIAIASNANVLKNANAVLVYENDHFRIQAATGEIEHFVTSLSIDPRVRGACSGGYCRSVLMDGSVLMSYLSIEEMDSIAQHQIEANMGNTPWNSIWRTEMNRVALYRRAAKDWRQLIKATPEMQSSLLDTEF

SEQ ID NO: 491 - protein, 201 aa, unidentified
  source 1..201: note = Candidatus Dependentiae bacterium
CHVLNFQTDYKGEIKLAHKYSVRKIIDIYAKVVRDGDVLEIRVENGSQIVNFNPKVFNDGKIIGAFAVVKFVDGSLLYETMSKSEIDHTRVTFSKMPNGMAWKDSEGEMCRKTVLRRICKLIDLHFDSVEQEQAWNDGSDADLTKNEPVKPEIQNPFPTKAVEAVIVTEEEKLRKQLKDKDPTLQDWQIDALVREHKEANQ

SEQ ID NO: 492 - DNA, 20 bp, Synthetic construct
  source 1..20: mol_type = other DNA
ccaccgcaaatgcttctagg

SEQ ID NO: 493 - DNA, 20 bp, Synthetic construct
  source 1..20: mol_type = other DNA
gcttgctgatccacatctgc

SEQ ID NO: 494 - DNA, 20 bp, Synthetic construct
  source 1..20: mol_type = other DNA
agtagactaatctctggctg

SEQ ID NO: 495 - DNA, 21 bp, Synthetic construct
  source 1..21: mol_type = other DNA
gatgtgtcgtcatctccttca

SEQ ID NO: 496 - DNA, 20 bp, Synthetic construct
  source 1..20: mol_type = other DNA
cacagtggggccactaggga

SEQ ID NO: 497 - DNA, 20 bp, Synthetic construct
  source 1..20: mol_type = other DNA
gagccacattaaccggccct

SEQ ID NO: 498 - DNA, 20 bp, Synthetic construct
  source 1..20: mol_type = other DNA
cggatccagctcagccatgg

SEQ ID NO: 499 - DNA, 20 bp, Synthetic construct
  source 1..20: mol_type = other DNA
ggcggtcccgcgctggggaa

SEQ ID NO: 500 - DNA, 20 bp, Synthetic construct
  source 1..20: mol_type = other DNA
ggtagtcgtactcgtcgtcg

SEQ ID NO: 501 - DNA, 20 bp, Synthetic construct
  source 1..20: mol_type = other DNA
tgggctttaagacgcttact

SEQ ID NO: 502 - DNA, 20 bp, Synthetic construct
  source 1..20: mol_type = other DNA
taaacacgtgttcctaatcc

SEQ ID NO: 503 - DNA, 20 bp, Synthetic construct
  source 1..20: mol_type = other DNA
tggacaagaaggaagagtaa

SEQ ID NO: 504 - DNA, 20 bp, Synthetic construct
  source 1..20: mol_type = other DNA
gacagaaatcccacagcagt

SEQ ID NO: 505 - DNA, 24 bp, Synthetic construct
  source 1..24: mol_type = other DNA
aatagagcccctgctgctgtgaag

SEQ ID NO: 506 - DNA, 19 bp, Synthetic construct
  source 1..19: mol_type = other DNA
gccctgcctcgccacatga

SEQ ID NO: 507 - DNA, 24 bp, Synthetic construct
  source 1..24: mol_type = other DNA
gataccatgtctttcgtgagcgcc

SEQ ID NO: 508 - DNA, 17 bp, Synthetic construct
  source 1..17: mol_type = other DNA
agtgccctgggccacgc

SEQ ID NO: 509 - DNA, 19 bp, Synthetic construct
  source 1..19: mol_type = other DNA
agggtcgatggtcaggggc

SEQ ID NO: 510 - DNA, 24 bp, Synthetic construct
  source 1..24: mol_type = other DNA
aatcacagagtactcgccggtcag

SEQ ID NO: 511 - DNA, 31 bp, Synthetic construct; identical to SEQ ID NO: 44
  source 1..31: mol_type = other DNA

SEQ ID NO: 512 - DNA, 22 bp, Synthetic construct
  source 1..22: mol_type = other DNA
ggagaataagaccatgtactgc

SEQ ID NO: 513 - DNA, 23 bp, Synthetic construct
  source 1..23: mol_type = other DNA
gaggatgaactagagacaaaagg

SEQ ID NO: 514 - DNA, 17 bp, Synthetic construct
  source 1..17: mol_type = other DNA
tcagtgccttcggactg

SEQ ID NO: 515 - DNA, 18 bp, Synthetic construct
  source 1..18: mol_type = other DNA
aaaggccataggcaggac

SEQ ID NO: 516 - DNA, 19 bp, Synthetic construct
  source 1..19: mol_type = other DNA
actgtctattggaagcgga

SEQ ID NO: 517 - DNA, 22 bp, Synthetic construct
  source 1..22: mol_type = other DNA
ggacagctggttaggaattaag

SEQ ID NO: 518 - DNA, 20 bp, Synthetic construct
  source 1..20: mol_type = other DNA
ggaagcggagctactaactt

SEQ ID NO: 519 - DNA, 22 bp, Synthetic construct
  source 1..22: mol_type = other DNA
ttaggaattaagtttgtgcccc

SEQ ID NO: 520 - DNA, 19 bp, Synthetic construct
  source 1..19: mol_type = other DNA
aaatgccaccccttgaagg

SEQ ID NO: 521 - DNA, 24 bp, Synthetic construct
  source 1..24: mol_type = other DNA
atcagaggaattgtagagtactga

SEQ ID NO: 522 - DNA, 19 bp, Synthetic construct
  source 1..19: mol_type = other DNA
acacatcacgcatggaaga

SEQ ID NO: 523 - DNA, 19 bp, Synthetic construct
  source 1..19: mol_type = other DNA
ggtaagtcatccctcagcc

SEQ ID NO: 524 - DNA, 18 bp, Synthetic construct
  source 1..18: mol_type = other DNA
agaagtagacggaagcgg

SEQ ID NO: 525 - DNA, 25 bp, Synthetic construct
  source 1..25: mol_type = other DNA
agccacagatttaggaattaagttt

SEQ ID NO: 526 - DNA, 18 bp, Synthetic construct
  source 1..18: mol_type = other DNA
tgtggtgtgtggggagct

SEQ ID NO: 527 - DNA, 26 bp, Synthetic construct
  source 1..26: mol_type = other DNA
ttacacgaaagcaatgctatcacctc

SEQ ID NO: 528 - DNA, 43 bp, Synthetic construct; identical to SEQ ID NO: 63
  source 1..43: mol_type = other DNA

SEQ ID NO: 529 - DNA, 43 bp, Synthetic construct; identical to SEQ ID NO: 64
  source 1..43: mol_type = other DNA

SEQ ID NO: 530 - DNA, 44 bp, Synthetic construct
  source 1..44: mol_type = other DNA
cctctctatgggcagtcggtgatggtacagcttcatgtgcatgt

SEQ ID NO: 531 - DNA, 41 bp, Synthetic construct
  source 1..41: mol_type = other DNA
ccatctcatccctgcgtgtctccgaggccgacaaagagaca

SEQ ID NO: 532 - DNA, 43 bp, Synthetic construct
  source 1..43: mol_type = other DNA
ccatctcatccctgcgtgtctccgtaaatgctgctctcttccc

SEQ ID NO: 533 - DNA, 45 bp, Synthetic construct
  source 1..45: mol_type = other DNA
cctctctatgggcagtcggtgatgttgtgaaagtgccacaaaaca

SEQ ID NO: 534 - DNA, 44 bp, Synthetic construct
  source 1..44: mol_type = other DNA
ccatctcatccctgcgtgtctccctactgctgatgataccagtg

SEQ ID NO: 535 - DNA, 44 bp, Synthetic construct
  source 1..44: mol_type = other DNA
cctctctatgggcagtcggtgatggttgtcatgccatacagact

SEQ ID NO: 536 - DNA, 20 bp, Synthetic construct
  source 1..20: mol_type = other DNA
agtcctgtggcatccacgaa

SEQ ID NO: 537 - DNA, 22 bp, Synthetic construct
  source 1..22: mol_type = other DNA
ggcacgaaggctcatcattcaa

SEQ ID NO: 538 - DNA, 22 bp, Synthetic construct
  source 1..22: mol_type = other DNA
agacacatgctaacaggatcta

SEQ ID NO: 539 - DNA, 22 bp, Synthetic construct
  source 1..22: mol_type = other DNA
ggcacgaaggctcatcattcaa

SEQ ID NO: 540 - DNA, 20 bp, Synthetic construct
  source 1..20: mol_type = other DNA
aggccatgggaatcatgaac

SEQ ID NO: 541 - DNA, 22 bp, Synthetic construct
  source 1..22: mol_type = other DNA
ctcacgttagaacgccattaca

SEQ ID NO: 542 - DNA, 19 bp, Synthetic construct
  source 1..19: mol_type = other DNA
aggcctttgggtgcagctg

SEQ ID NO: 543 - DNA, 19 bp, Synthetic construct
  source 1..19: mol_type = other DNA
cagtccacctgctccaccc

SEQ ID NO: 544 - DNA, 21 bp, Synthetic construct
  source 1..21: mol_type = other DNA
gggtagctcctgaaccattgt

SEQ ID NO: 545 - DNA, 22 bp, Synthetic construct
  source 1..22: mol_type = other DNA
ccagaagcactcaaacatgctg

SEQ ID NO: 546 - DNA, 18 bp, Synthetic construct
  source 1..18: mol_type = other DNA
ctgccggaaatggcgcag

SEQ ID NO: 547 - DNA, 21 bp, Synthetic construct
  source 1..21: mol_type = other DNA
gtaggagacggaaccgcacaa

SEQ ID NO: 548 - DNA, 1548 bp, Synthetic construct
  source 1..1548: mol_type = other DNA
agtgacctgtgtaattatgcagaagaatggagctggattacacacagcaagttcctgcttctgggacagctctactgacggtatgattttcattcatgtttgtgaagttttgttgtgtgaaatatatgactggaagtttcctatctttgaatgcaatgcatgtttatcaccttttaaaacatttaataatagacttgccaaggttctttgtgtagcatagagatgggtacttgaatgttggccttattgtgagtaaaacgtcgtcccccagctttccctgccgtaaatgctgctctcttccctcccgcagggagctgcactgtgcgatgggagaataagaccatgtactgcatcgtcagtgccttcggactgtctattggaagcggagctactaacttcagcctgctgaagcaggctggagacgtggaggagaaccctggacctgccaccgtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattcctaaccagctgtcctgcctatggcctttctccttttgtctctagttcatcctctaaccaccagccatgaattcagtgaactcttttctcattctctttgttttgtggcactttcacaatgtagaggaaaaaaccaaatgaccgcactgtgatgtgaatggcaccgaagtcagatgagtatccctgtaggtcacctgcagcctgcgttgccacttgtcttaactctgaatatttcatttcaaaggtgctaaaatctgaaatctgctagtgtgaaacttgctctactctctgaaatgattcaaatacactaattttccatactttatacttttgttagaataaattattcaaatctaaagtctgttgtgttcttcatagtctgcatagtatcataaacg

SEQ ID NO: 549 - DNA, 1973 bp, Synthetic construct; identical to SEQ ID NO: 82
  source 1..1973: mol_type = other DNA

SEQ ID NO: 550 - DNA, 2193 bp, Synthetic construct; identical to SEQ ID NO: 83
  source 1..2193: mol_type = other DNA

SEQ ID NO: 551 - DNA, 1245 bp, Synthetic construct; identical to SEQ ID NO: 84
  source 1..1245: mol_type = other DNA

SEQ ID NO: 552 - DNA, 1357 bp, Synthetic construct
  source 1..1357: mol_type = other DNA
tgtggtgtgtggggagctgtcacatccagggtcctcactgcctgtccccttccctcctcagatcattgctcctcctgagcgcaagtactccgtgtggatcggcggctccatcctggcctcgctgtccaccttccagcagatgtggatcagcaagcaggagtatgacgagtccggcccctccatcgtccaccgcaagtgtttcggaagcggagctactaacttcagcctgctgaagcaggctggagacgtggaggagaaccctggacctgtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattcctaataggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaa

SEQ ID NO: 553 - DNA, 1400 bp, Synthetic construct; identical to SEQ ID NO: 79
  source 1..1400: mol_type = other DNA

SEQ ID NO: 554 - DNA, 1986 bp, Synthetic construct
  source 1..1986: mol_type = other DNA
tgccgtaaatgctgctctcttccctcccgcagggagctgcactgtgcgatgggagaataagaccatgtactgcatcgtcagtgccttcggactgtctattggaagcggagctactaacttcagcctgctgaagcaggctggagacgtggaggagaaccctggacctgccaccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattccgctagcggcagtggagagggcagaggaagtctgctaacatgcggtgacgtcgaggagaatcctggcccaggtggttctgccggtggctccggttctggctccagcggtggcagctctggtgcgtccggcacgggtactgcgggtggcactggcagcggttccggtactggctctggcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaccagctgtcctgcctatggcctttctccttttgtctctagttcatcctctaaccaccagccatgaattcagtgaactcttttctcattctctttgttttgtggcactttcacaatgtagaggaaaaaaccaaatgaccgcactgtgatgtgaatggcaccgaagtcagatgagtatccctgtaggtcacctgcagcctgcgttgccacttgtctt

SEQ ID NO: 555 - DNA, 2069 bp, Synthetic construct
  source 1..2069: mol_type = other DNA
tactgtcttgaaagcagatagaaaccaagagtattaccctaatagctggctttaagaaatctttgtaatatgaggattttattttggaaacaggtattgatgaagatgaccctactgctgatgataccagtgctgctgtaactgaagaaatgccaccccttgaaggagatgacgacacatcacgcatggaagaagtagacggaagcggagctactaacttcagcctgctgaagcaggctggagacgtggaggagaaccctggacctgccaccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattccgctagcggcagtggagagggcagaggaagtctgctaacatgcggtgacgtcgaggagaatcctggcccaggtggttctgccggtggctccggttctggctccagcggtggcagctctggtgcgtccggcacgggtactgcgggtggcactggcagcggttccggtactggctctggcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaatctgtggctgagggatgacttacctgttcagtactctacaattcctctgataatatattttcaaggatgtttttctttatttttgttaatattaaaaagtctgtatggcatgacaactactttaaggggaagataagatttctgtctactaagtgatgctgtgataccttaggcactaaagcagagctagtaatgct

SEQ ID NO: 556 - DNA, 1396 bp, Synthetic construct
  source 1..1396: mol_type = other DNA
aggccatgggaatcatgaactccttcgtcaacgacatcttcgaacgcatcgcgggtgaggcttcccgcctggcgcattacaacaagcgctcgaccatcacctccagggagatccagacggccgtgcgcctgctgctgcccggggagttggccaagcacgccgtgtccgagggcaccaaggccgtcaccaagtacaccagcgctaagagatctggaagcggagctactaacttcagcctgctgaagcaggctggagacgtggaggagaaccctggacctatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattcctaataaacttggaaagtaagcgtcttaaagcccaaccccaaaggctcttttaagagccacttaaattatcgatattagagctgtaaacacgtgttcctaatccaggcttaaattcgggcgatctttttaaagggattatcatgatcttatcacactgagtaatgcatgcagtgcttgtaatcgagtctggctaaggagaagccacatacactttgttagtactgagtgaggaatatggcgcctaaaaaattagggatgtgcaggaaacttgtgttaacagaaagtgcttcctggctcttggcgcgaaaatggatacttccgttgtgtcgagtgctgtgacttcctgttagaacttgttgaaagcctattgtgtcacgtgtactttccaccatgtaatggcgttctaacgtgag

SEQ ID NO: 557 - DNA, 1381 bp, Synthetic construct
  source 1..1381: mol_type = other DNA
aggcctttgggtgcagctggggagggggccccttgttcacttgaatagctgttgttaggagagaggggaaccgaggtggacctctggggcatggggctggaggtggcaggggaggagtggacccggccaacctactgctgtgggatttctgtccctttccaggctgcagtagatggtcccatggacaagaaggaagagagatctggaagcggagctactaacttcagcctgctgaagcaggctggagacgtggaggagaaccctggacctatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattcctaataagggcctccttcctcccctgcctgcagctggcttccacctggcacgtgcctgctgcttcctgagagcccggcctctccctccagtacttctgtttgtgcccttctgcttcccccattcccttccacagctcatagctcgtcatctcggcccttgtccacactctccaagcacattacaggggacctgattgctacacgttcagaatgcgtttgctgtcatcctgcttggcctggccaggcctggcacagccttggcttccacgcctgagcgtggagagcacgagttagttgtagtccggcttgcggtggggctgacttcctgttggtttgagcccctttttgttttgccctctgggtgttttctttggtcccgcaggagggtgggtggagcaggtggactg

SEQ ID NO: 558 - DNA, 1436 bp, Synthetic construct
  source 1..1436: mol_type = other DNA
gggtagctcctgaaccattgttgtcctctgattggttgttcccttttcggctctgcaacaccgcctagaccgaccggatacacgggtagggcttccgctttacccgtctccctcctggcgcttgtcctcctctcccagtcggcaccacagcggtggctgccgggcgtggtgtcggtgggtcggttggtttttgtctcaccgttggtgtccgtgccgttcagttgcccgcccatatgatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattccagatctggaagcggagctactaacttcagcctgctgaagcaggctggagacgtggaggagaaccctggacctatggctgagctggatccgttcggcgcccctgccggcgcccctggcggtcccgcgctggggaacggagtggccggcgccggcgaagaagacccggctgcggccttcttggcgcagcaagagagcgagattgcgggcatcgagaacgacgaggccttcgccatcctggacggcggcgcccccgggccccagccgcacggcgagccgccggggggtccgggtgagagtgcgggcgcgtttggggcgagaggacttgtctggaaactcggtccacagtgggtccgagagcttctgtgtgactcgtgctccttgctgaattaggaggttagggagcagtgcaaacaggaaacgagaccctggcccggtctttcagaaacctaggctcgagaagcctgttcggttctcagcatgtttgagtgcttctgg

SEQ ID NO: 559 - DNA, 1025 bp, Synthetic construct
  source 1..1025: mol_type = other DNA
ctgccggaaatggcgcagcggcagggaggggctcttcacccagtccggcagttgaagctcggcgctcgggttacccctgcagcgacgccccctggtcccacagataccactgctgctcccgccctttcgctcctcggccgcgcaatgggcgtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattccacacgtgacgacgagtacgactacctctttaaaggtgaggccatgggctctcgcactctacacagtcctcgttcggggacccgggccactcccggtggaccctcgtgccggccacccctgcactgatataggcctccctcagcccttcctttttgtgcggttccgtctcctac

SEQ ID NO: 560 - DNA, 16 bp, Synthetic construct
  source 1..16: mol_type = other DNA
gtaaaacgacggccag

SEQ ID NO: 561 - DNA, 17 bp, Synthetic construct
  source 1..17: mol_type = other DNA
caggaaacagctatgac

SEQ ID NO: 562 - DNA, 200 bp, Synthetic construct
  source 1..200: mol_type = other DNA
caccttcagattttcctgtaacgatcgggaactggcatcttcagggagtagctgacctcttctccttctcccacaggatcctggagccacccgcagttcgaaaagctcagtgaagagaagaacaaaaagcagcatattacagttagttgtcttcatcaatctttaaatatgttgtgtggtttttctctccctgtttccac

SEQ ID NO: 563 - DNA, 80 bp, Synthetic construct
  source 1..80: mol_type = other DNA
atgcaatgcatgtttatcaccttttaaaacatttaataatagacttgccaaggttctttgtgtagcatagagatgggtac

SEQ ID NO: 564 - DNA, 80 bp, Synthetic construct
  source 1..80: mol_type = other DNA
gaccatgtactgcatcgtcagtgccttcggactgtctattccatggggaagcggagctactaacttcagcctgctgaagc

SEQ ID NO: 565 - DNA, 80 bp, Synthetic construct
  source 1..80: mol_type = other DNA
cgacctccctagcaaactggggcacaaacttaattcctaaccatggccagctgtcctgcctatggcctttctccttttgt

SEQ ID NO: 566 - DNA, 80 bp, Synthetic construct
  source 1..80: mol_type = other DNA
agatgagtatccctgtaggtcacctgcagcctgcgttgccacttgtcttaactctgaatatttcatttcaaaggtgctaa

SEQ ID NO: 567 - DNA, 60 bp, Synthetic construct
  source 1..60: mol_type = other DNA
catcgtcagtgccttcggactgtctattccatggggaagcggagctactaacttcagcct

SEQ ID NO: 568 - DNA, 60 bp, Synthetic construct
  source 1..60: mol_type = other DNA
gacgacacatcacgcatggaagaagtagacggaagcggagctactaacttcagcctgctg

SEQ ID NO: 569 - DNA, 62 bp, Synthetic construct
  source 1..62: mol_type = other DNA
actggggcacaaacttaattcctaaatctgtggctgagggatgacttacctgttcagtactc

SEQ ID NO: 570 - DNA, 20 bp, Synthetic construct
  source 1..20: mol_type = other DNA
caaagtaccccgtctccctg

SEQ ID NO: 571 - DNA, 107 bp, Synthetic construct
  source 1..107: mol_type = other DNA
gcctccccttcttgtaggcctgcatcatcaccgtttttctggacaaccccaaagtaccccgtctccctggcttagccacctctccatcctcttgctttctttgcctg

SEQ ID NO: 572 - DNA, 108 bp, Synthetic construct
  source 1..108: mol_type = other DNA
caggcaaagaaagcaagaggatggagaggtggctaaagccagggagacggggtactttggggttgtccagaaaaacggtgatgatgcaggcctacaagaaggggaggc

SEQ ID NO: 573 - DNA, 109 bp, Synthetic construct
  source 1..109: mol_type = other DNA
cgcctccccttcttgtaggcctgcatcatcaccgtttttctggacaaccccaaagtaccccgtctccctggctttagccacctctccatcctcttgctttctttgcctg

SEQ ID NO: 574 - DNA, 96 bp, Synthetic construct
  source 1..96: mol_type = other DNA
gctctggttctgggtacttttatctgtcccctccaccccacagtggggcaagcttctgacctcttctcttcctcccacagggcctcgagagatctg

SEQ ID NO: 575 - DNA, 96 bp, Synthetic construct
  source 1..96: mol_type = other DNA
cagatctctcgaggccctgtgggaggaagagaagaggtcagaagcttgccccactgtggggtggaggggacagataaaagtacccagaaccagagc

SEQ ID NO: 576 - DNA, 100 bp, Synthetic construct
  source 1..100: mol_type = other DNA
tacttgaatgttggccttattgtgagtaaaacgtcgtcccccagctttccctgccgtaaatgctgctctcttccctcccgcagggagctgcactgtgcga

SEQ ID NO: 577 - DNA, 100 bp, Synthetic construct
  source 1..100: mol_type = other DNA
tcgcacagtgcagctccctgcgggagggaagagagcagcatttacggcagggaaagctgggggacgacgttttactcacaataaggccaacattcaagta

SEQ ID NO: 578 - DNA, 99 bp, Synthetic construct
  source 1..99: mol_type = other DNA
ggagaataagaccatgtattgtattgtcagtgccttcggactgtctattccatggggaagcggagctactaacttcagcctgctgaagcaggctggaga

SEQ ID NO: 579 - DNA, 102 bp, Synthetic construct
  source 1..102: mol_type = other DNA
tcctatctttgaatgcaatgcatgtttatcaccttttaaaacatttaataatagacttgccaaggttctttgtgtagcatagagatgggtacttgaatgttg

SEQ ID NO: 580 - DNA, 102 bp, Synthetic construct
  source 1..102: mol_type = other DNA
caacattcaagtacccatctctatgctacacaaagaaccttggcaagtctattattaaatgttttaaaaggtgataaacatgcattgcattcaaagatagga

SEQ ID NO: 581 - DNA, 100 bp, Synthetic construct
  source 1..100: mol_type = other DNA
gggagaataagacgatgtattgtattgtcagtgccttcggactgtctattccatggggaagcggagctactaacttcagcctgctgaagcaggctggaga

SEQ ID NO: 582 - DNA, 100 bp, Synthetic construct
  source 1..100: mol_type = other DNA
tctccagcctgcttcagcaggctgaagttagtagctccgcttccccatggaatagacagtccgaaggcactgacaatacaatacatcgtcttattctccc

SEQ ID NO: 583 - DNA, 100 bp, Synthetic construct
  source 1..100: mol_type = other DNA
gggagaataagaccatgtattgtattgtcagtgccttcggactgtctattccatggggaagcggagctactaacttcagcctgctgaagcaggctggaga

SEQ ID NO: 584 - DNA, 60 bp, Synthetic construct
  source 1..60: mol_type = other DNA
actttgagtgtagcagagaggaaccattgccaccttcagattttcctgtaacgatcggga

SEQ ID NO: 585 - DNA, 54 bp, Synthetic construct
  source 1..54: mol_type = other DNA
tccgggacaccctccttctcttctccagtcttcgaacggggtgacaccccacct

(19) United States
(12) Patent Application Publication
(10) Pub. No.: US 2025/0034594 A1
(43) Pub. Date: Jan. 30, 2025

(54) RNA-GUIDED GENOME RECOMBINEERING AT KILOBASE SCALE

(71) Applicant: THE BOARD OF TRUSTEES OF THE LELAND STANFORD JUNIOR UNIVERSITY, Stanford, CA (US)

(72) Inventor: Le Cong, Stanford, CA (US)

(21) Appl. No.: 18/687,090

(22) PCT Filed: Sep. 1, 2022
(86) PCT No.: PCT/US22/75850

§ 371 Date: Feb. 27, 2024

Related U.S. Application Data
(60) Provisional application No. 63/239,732, filed on Sep. 1, 2021.

(51) Int. Cl. C12N 15/90; C12N 9/22; C12N 15/11; C12N 15/115

(52) U.S. Cl. CPC C12N 15/907; C12N 9/22; C12N 15/11; C12N 15/115; C12N 2310/16; C12N 2310/20

(57) ABSTRACT

The present disclosure provides recombineering-editing systems using CRISPR and recombination enzymes as well as methods, vectors, nucleic acid compositions, and kits thereof. The methods and systems provide means for altering target DNA, including genomic DNA in a host cell.

### RELATED APPLICATIONS AND INCORPORATION BY REFERENCE

This application is a national phase application under 35 U.S.C. § 371 of PCT International Application No.: PCT/US2022/075850, filed on Sep. 1, 2022, which claims the benefit of priority from U.S. Patent Application Ser. No. 63/239,732 filed Sep. 1, 2021, the contents of which are incorporated herein by reference in their entireties.

Reference is made to U.S. Patent Application Ser. No. 62/984,618, filed Mar. 3, 2020, U.S. Patent Application Ser. No. 63/146,447, filed Feb. 5, 2021, and PCT/US2021/020513, filed Mar. 2, 2021.

### SEQUENCE LISTING

The text of the computer readable sequence listing filed herewith, titled “STDU2_41203_601_SequenceListing_Updated_20240716”, created Jul. 16, 2024, having a file size of 668,877 bytes, is hereby incorporated by reference in its entirety.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to RNA-guided recombineering-editing systems using phage recombination enzymes as well as methods, vectors, nucleic acid compositions, and kits thereof.

### BACKGROUND OF THE INVENTION

The Clustered Regularly Interspaced Short Palindromic Repeats (CRISPR) system, originally found in bacteria and archaea as part of the immune system to defend against invading viruses, forms the basis for genome editing technologies that can be programmed to target specific stretches of a genome or other DNA for editing at precise locations. While various CRISPR-based tools are available, the majority are geared towards editing short sequences. Long-sequence editing is highly sought after in the engineering of model systems, therapeutic cell production and gene therapy. Prior studies have developed technologies to improve Cas9-mediated homology-5 directed repair (HDR) (K. S. Pawelczak, et al., ACS Chem. Biol. 13, 389-396 (2018)), and tools leveraging nucleic acid modification enzymes with Cas9, e.g., prime-editing (A. V. Anzalone, et al., Nature. 576, 149-157 (2019)) that demonstrated editing up to 80 base-pairs (bp) in length. Despite these progresses, there are continued demands for large-scale mammalian genome engineering with high efficiency and fidelity.

### SUMMARY OF THE INVENTION

Provided herein are systems and methods that facilitate nucleic acid editing in a manner that allows large-scale nucleic acid editing with high accuracy and low off-target errors. These systems and methods employ a combination of recombination components with CRISPR recombination components.

For example, disclosed herein are systems comprising a protein, a nucleic acid molecule comprising a guide RNA sequence that is complementary to a target DNA sequence, and a recombination protein. A recombination protein may comprise an exonuclease, a single stranded DNA binding protein (SSB), a single stranded DNA annealing protein (SSAP), or functional fragment or activity thereof. A recombination protein may comprise or be engineered to comprise a two or more of the activities. In certain embodiments, recombination proteins are cooperative. In certain embodiments the recombination protein comprises a microbial recombination protein, for example a bacterial or bacteriophage protein, including but not limited to, RecE, RecT, lambda exonuclease (Exo), Bet protein (betA, redB), exonuclease gp6, single-stranded DNA-binding protein gp2.5, or a derivative or variant thereof. In certain embodiments the recombination protein comprises a eukaryotic or mammalian recombination protein. A certain non-limiting embodiments, a eukaryotic recombination protein or system comprises RAD52 or a homolog thereof which binds ssDNA, and mediates annealing of complementary ssDNA, including RPA-bound complementary ssDNA. In some embodiments, the system further comprises donor DNA. In some embodiments, the target DNA sequence is a genomic DNA sequence in a host cell.

In certain embodiments, the invention provides a system comprising one, two, three, or more recombination proteins of SEQ ID NO:166 to SEQ ID NO:491 or a recombination protein at least 85%, at least 90%, at least 95% identical, or higher thereto. In certain embodiments, the recombination protein has at least 85% identity to a recombination protein of Table 9. In certain embodiments, the recombination protein has at least 85% identity to SEQ ID NO:179, SEQ ID NO: 185, SEQ ID NO:205, SEQ ID NO:321, SEQ ID NO: 353, SEQ ID NO:359, SEQ ID NO:366, SEQ ID NO:424, or SEQ ID NO:479.

In certain embodiments, the recombination protein has at least 95% identity to SEQ ID NO: 166, SEQ ID NO: 168, SEQ ID NO: 169, SEQ ID NO: 170, SEQ ID NO: 171, SEQ ID NO:241, SEQ ID NO:253, SEQ ID NO:290, SEQ ID NO:408, SEQ ID NO:411, or SEQ ID NO:442.

In some embodiments, the system further comprises a recruitment system comprising at least one aptamer sequence and an aptamer binding protein functionally linked to the recombination protein as part of a fusion protein. In some embodiments, the aptamer sequence is an RNA aptamer sequence or a peptide aptamer sequence. In some embodiments, the RNA aptamer sequence is part of the nucleic acid molecule. In some embodiments, the nucleic acid molecule comprises two RNA aptamer sequences. In some embodiments, the recombination protein is functionally linked to the aptamer binding protein as a fusion protein. In some embodiments, the binding protein comprises a MS2 coat protein, a lambda N22 peptide, or a functional derivative, fragment, or variant thereof. In some embodiments, the fusion protein further comprises a linker and/or a nuclear localization sequence.

Without being bound by theory, the recruitment system serves to localize one or more recombination proteins to the location of a Cas protein/gRNA complex and via interaction between recombinase and a template nucleic acid promote HDR at a selected target while not promoting off-target Cas protein function.

The recruitment system is adaptable to a multitude of combinations and configurations of recombination proteins. For example, by selecting and incorporating multiple nucleic acid aptamers, the system can comprise multiple recombination proteins, which may be the same or different and in various ratios. In certain embodiments, the system comprises an exonuclease. In certain embodiments, the system comprises an SSAP. In certain embodiments, the system comprises an SSB. In certain embodiments, the system comprises an exonuclease and an SSAP.

In certain embodiments, the system comprises an exonuclease and an SSB. In certain embodiments, the system comprises an SSAP and an SSB. In certain embodiments, the system comprises an exonuclease and an SSAP and does not comprise an SSB. In certain embodiments, the system comprises an exonuclease and an SSB and does not comprise an SSAP. In certain embodiments, the system comprises an SSAP and an SSB and does not comprise an exonuclease. In certain embodiments, the system comprises an exonuclease, an SSAP, and an SSB.

Disclosed herein are compositions comprising a nucleic acid sequence encoding a fusion protein comprising a recombination protein functionally linked to an aptamer binding protein. The recombination protein may be a microbial recombination protein, including but not limited to RecE, RecT, lambda exonuclease (Exo), Bet protein (betA, redB), exonuclease gp6, single-stranded DNA-binding protein gp2.5, or a derivative or variant thereof. The compositions may further comprise one or both of a polynucleotide comprising a nucleic acid sequence encoding a Cas protein and a nucleic acid molecule comprising a guide RNA sequence that is complementary to a target DNA sequence. In some embodiments, the nucleic acid molecule further comprises at least one RNA aptamer sequence. In some embodiments, the polynucleotide comprising a nucleic acid sequence encoding a Cas protein further comprises a sequence encoding at least one peptide aptamer sequence.

Also disclosed herein are vectors comprising a nucleic acid sequence encoding a fusion protein comprising a recombination protein functionally linked to an aptamer binding protein. A microbial recombination protein may comprise RecE, RecT, lambda exonuclease (Exo), Bet protein (betA, redB), exonuclease gp6, single-stranded DNA-binding protein gp2.5, or a derivative or variant thereof. The vectors may further comprise one or both of a polynucleotide comprising a nucleic acid sequence encoding a Cas protein and a nucleic acid molecule comprising a guide RNA sequence that is complementary to a target DNA sequence. In some embodiments, the nucleic acid molecule further comprises at least one RNA aptamer sequence. In some embodiments, the polynucleotide comprising a nucleic acid sequence encoding a Cas protein further comprises a sequence encoding at least one peptide aptamer sequence.

In certain embodiments, the fusion protein comprises a recombination protein comprising an amino acid sequence at least 75% similar, or at least 75% identical to a recombination protein of SEQ ID NO:166 to SEQ ID NO:491. In certain embodiments the fusion protein comprises a recombination protein comprising a sequence having at least 80%, at least 85%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% similarity or identity to a recombination protein of SEQ ID NO:166 to SEQ ID NO: 491.

In certain embodiments, systems comprising a recombination protein of the invention are capable of editing efficiency equal to or greater than systems comprising EcRecT, for example, without limitation, 1.2×, 1.5×, 1.7×, 2×, 2.5×, 3×, or more compared to EcRecT.

In certain embodiments, systems comprising a recombination protein of the invention provide cell viability equal to or greater than systems comprising EcRecT, for example, without limitation, 1.1×, 1.2×, 1.3×, 1.5×, 1.7×, 2×, 2.5×, 3×, or more compared to EcRecT.

In some embodiments, the Cas protein is Cas9 or Cas12a. In some embodiments, the Cas protein is a catalytically dead. In some embodiments, the Cas9 protein is wild-type Streptococcus pyogenes Cas9 or a wild type Staphylococcus aureus Cas9. In some embodiments, the Cas9 protein is a Cas9 nickase (e.g., wild-type Streptococcus pyogenes Cas9 with an amino acid substation at position 10 of D10A).

Also disclosed is a eukaryotic cell comprising the systems or vectors disclosed herein.

Further disclosed herein are methods of altering a target genomic DNA sequence in a host cell. The methods comprise contacting the systems, compositions, or vectors described herein with a target DNA sequence (e.g., introducing the systems, compositions, or vectors described herein into a host cell comprising a target genomic DNA sequence). Kits containing one or more reagents or other components useful, necessary, or sufficient for practicing any of the methods are also disclosed herein.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by the following Detailed Description.

Other aspects and embodiments of the disclosure will be apparent in light of the following detailed description and accompanying figures.

### BRIEF DESCRIPTION OF THE DRAWINGS

FIG. 1A and FIG. 1B are the reconstructed RecE (FIG. 1A) and RecT (FIG. 1B) phylogenetic trees with eukaryotic recombination enzymes from yeast and human.

FIG. 2A is a phylogenetic tree and length distribution of RecE/RecT homologs. FIG. 2B is the metagenomics distribution of RecE/T. FIG. 2C is a schematic showing central models disclosed herein. FIG. 2D are graphs of the genome knock-in efficiency of RecE/T homologs.

FIGS. 3A and 3B are graphs of the high-throughput sequencing (HTS) reads of homology directed repair (HDR) at the EMX1 (FIG. 3A) locus and the VEGFA (FIG. 3B) locus.

FIGS. 3C-3D are graphs of the mKate knock-in efficiency at HSP90AA1 (FIG. 3C), DYNLT1 (FIG. 3D), and AAVS1 (FIG. 3E) loci in HEK293T cells. FIG. 3F is images of mKate knock-in efficiency in HEK293T cells with RecT. FIG. 3G is a schematic of an exemplary AAVS1 knock-in strategy and chromatogram trace from RecT knock-in group. FIG. 3H is schematics and graphs of the recruitment control experiment and corresponding knock-in efficiency. All results are normalized to NR. (NC, no cutting; NR, no recombinator).

FIGS. 4A-4C are graphs of the relative mKate knock-in efficiencies to the NE group at HSP90AA1 (FIG. 4A), DYNLT1 (FIG. 4B), and AAVS1 (FIG. 4C) loci in HEK293T cells. (NC, no cutting control group. NR, no recombinator control group.) FIG. 4D is an image of an exemplary agarose gel of junction PCR that validates mKate knock-in at AAVS1 locus. FIGS. 4E and 4F arc graphs of the absolute and (FIG. 4E) and relative (FIG. 4F) LOV knock-in efficiencies at AAVS1 locus. FIG. 4G are the Sanger sequencing results of the junction PCR product of an exemplary mKate knock-in at AAVS1 locus.

FIGS. 5A-5D are graphs of the genomic knock-in efficiencies at different loci across cell lines A549 (FIG. 5A), HepG2 (FIG. 5B), HeLa (FIG. 5C), and hESCs (H9) (FIG. 5D). FIG. 5E is images of mKate knock-ins in hESCs. FIGS. 5F and 5G are genomic-wide off-target site (OTS) counts (FIG. 5F) and OTS chromosomal distribution (FIG. 5G) of REDITv1 tools.

FIGS. 6A-6D are graphs of the relative mKate knock-in efficiency at the AAVS1 locus and the DYNT1 locus in A549 cell line (FIG. 6A), the DYNLT1 locus and the HSP90AA1 locus in HepG2 cell line (FIG. 6B), the DYNLT1 locus and the HSP90AA1 locus in Hela cell line (FIG. 6C), and the HSP90AA/locus and the OCT4 locus in hES-H9 cell line (FIG. 6D). (NC, no cutting control group. NR, no recombinator control group. All data normalized to NR group.) FIG. 6E is representative FACS results of HSP90AA1 mKate knock-in in hES-H9 cells.

FIGS. 7A-7D are graphs of the absolute mKate knock-in efficiencies of different homology arm lengths at the DYNLT1 (FIG. 7A) and HSP90AA1 (FIG. 7B) loci and the no recombinator controls for DYNLT1 (FIG. 7C) and HSP90AA1 (FIG. 7D).

FIGS. 8A-8E are graphs of the indel rates of the top 3 predicted off-target loci associated with sgEMX1 (FIGS. 8A-8C) or sgVEGFA (FIGS. 8D-8E) in the REDITv1 system.

FIG. 9A is a schematic of select embodiments of REDITv2N and corresponding knock-in efficiencies in HEK293T cells. FIGS. 9B and 9C are graphs of genomic-wide off-target site (OTS) counts (FIG. 9B) and OTS chromosomal distribution (FIG. 9C) comparing REDITv2N against REDITv1. FIG. 9D is a schematic of select embodiments of REDITv2D and corresponding knock-in efficiencies. FIG. 9E is a graph of editing efficiency of REDITv1, REDITv2N, and REDITv2D under serum starvation conditions. FIG. 9F is the knock-in efficiencies of REDITv3 in hESCs. FIG. 9G is images of mKate knock in using REDITv3 in hESCs.

FIGS. 10A and 10B are schematics and graphs of the relative mKate knock-in efficiencies of select embodiments of REDITv2N (FIG. 10A) and REDITv2D (FIG. 10B) at the DYNLT1 locus and the HSP90AA1 locus.

FIGS. 11A-11D are images of agarose gels showing junction PCR of mKate knock-in at the DYNLT1 locus and the HSP90AA1 locus for a select REDITv2N system. FIG. 11E is the chromatogram sequence of junction PCR products at the DYNLT1 locus.

FIGS. 12A and 12B are graphs of the genomic distribution of detected off-target cleavages of select embodiments of REDITv2 (FIG. 12A) and REDITv2N (FIG. 12B). A pileup includes alignments that have two or more reads overlapping with each other. Flanking pairs include alignments that show up on opposite strands within 200 bp upstream of each other. Target matched includes alignments that match to a treated target in the upstream sequence (up to 6 mismatches, including 1 mismatch in the PAM, are allowed in the target sequence). FIG. 12C is a graph of the HTS HDR and indel reads at EMX1 locus for REDITv2N system.

FIG. 13A is an image of an agarose gel showing junction PCR of mKate knock-ins at the DYNLT1 locus for REDITv2D system. FIG. 13B is the chromatogram sequence of junction PCR products at the DYNLT1 locus.

FIGS. 14A-14C are graphs of the mKate knock-in efficiencies at the HSP90AA1 locus in REDITv2 (FIG. 14A), REDITv2N (FIG. 14B) and REVITv2D (FIG. 14C) when treated with different FBS concentrations. FIGS. 14A-14C are graphs of the mKate knock-in efficiencies at the HSP90AA1 locus in REDITv2 (FIG. 14D), REDITv2N (FIG. 14E) and REVITv2D (FIG. 14F) when treated with different serum FBS concentrations.

FIG. 15 is images of the nuclear localization of RecE_587 and RecT following EGFP fusion to the REDITv1 systems. Nuclei were stained with NucBlue Live Ready Probes Reagent.

FIGS. 16A and 16B are the relative mKate knock-in efficiencies at HSP90AA1 and DYNLT1 loci following fusion of different nuclear localization sequences to either the N- or C-terminus of RecT and RecE_587. FIGS. 16C and 16D are graphs of the absolute mKate knock-in efficiencies of the constructs from FIGS. 16A and 16B for the DYNLT1 locus (FIG. 16C) and the HSP90AA1 locus (FIG. 16D).

FIGS. 17A-17D are graphs of the relative (FIGS. 17A and 17B) and absolute (FIGS. 17C and 17D) mKate knock-in efficiencies for the DYNLT1 locus (FIGS. 17A and 17C) and the HSP90AA1 locus (FIGS. 17B and 17D) following fusion new NLS sequences as well as optimal linkers to REDITv2 and REDITv3 variants. The REDITv2 versions using REDITv2N (D10A or H840A) and REDITv2D (dCas9) are indicated in the horizonal axis, along with the number of guides used. The different colors represent the different control groups and REDIT versions.

FIG. 18 is a graph of the relative editing efficiency of REDITv3N system at HSP90AA1 locus in hES-H9 cells.

FIG. 19A is a diagram of an exemplary saCas9 expression vector. FIGS. 19B-19E are graphs of the relative mKate knock-in efficiencies at the AAVS1 locus (FIG. 19B) and HSP90AA1 locus (FIG. 19C) of different effectors in saCas9 system and the respective absolute efficiencies (FIGS. 19D and 19E, respectively). (NC, no cutting control group. NR, no recombinator control group.

FIG. 20A is a schematic of RecT truncations. FIGS. 20B and 20C are graphs of the relative mKate knock-in efficiencies at the DYNLT1 locus for wild-type Streptococcus pyogenes Cas9 and Streptococcus pyogenes Cas9n (D10A) with single- and double-nicking.

FIG. 21A is a schematic of RecE_587 truncations. FIGS. 21B and 21C are graphs of the relative mKate knock-in efficiencies at the DYNLT1 locus for wild-type Streptococcus pyogenes Cas9 and Streptococcus pyogenes Cas9n (D10A) with single- and double-nicking.

FIGS. 22A and 22B are graphs of comparison of efficiency to perform recombineering-based editing with various exonucleases (FIG. 22A) and single-strand DNA annealing protein (SSAP) (FIG. 22B) from naturally occurring recombineering systems, including NR (no recombinator) as negative control. The gene-editing activity was measured using mKate knock-in assay at genomic loci (DYNLT1 and HSP90AA1). The data shown are percentage of successful mKate knock-in using human HEK293 cells, each experiments were performed in triplicate (n=3).

FIGS. 23A-23E show a compact recruitment system using boxB and N22. The REDIT recombinator proteins were fused to N22 peptide and within the sgRNA was boxB, the short cognizant sequence of N22 peptide (FIG. 23A). FIGS. 23B-23E are graphs of the gene-editing efficiency using mKate knock-in assay, with wildtype SpCas9, with side-by-side comparisons to the MS2-MCP recruitment system. FIGS. 23B and 23D are absolute mKate knock-in efficiency at DYNLT1, HSP90AA1 loci and FIGS. 23C and 23E are relative efficiencies. The data shown are percentage of successful mKate knock-in using HEK293 human cells, each experiments were performed in triplicate (n=3).

FIGS. 24A-24C show a SunTag recruitment system. The REDIT recombinator proteins were fused to scFV antibody and the GCN4 peptide in tandem fashion (10 copies of GCN4 peptide separated by linkers) was fused to the Cas9 protein (FIG. 24A). An mKate knock-in experiment (FIG. 24B) with the DYNLT1 locus was used to measure the gene-editing knock-in efficiency (FIG. 24C). All data are measurements of gene-editing efficiency using mKate knock-in assay, with wildtype SpCas9. Absolute mKate knock-in efficiency at DYNLT1 are shown in the bottom right corner of each flow cytometry plot, where the control is without recombinator (NR), which included scFV fused to GFP protein as negative control, all experiments done in HEK293 human cells.

FIGS. 25A and 25B exemplify REDIT with a Cas12A system. A Cpf1/Cas12a based REDIT system via the SunTag recruitment design was created (FIG. 25A) for two different Cpf1/Cas12a proteins. Using the mKate knock-in assay, the efficiencies at two endogenous loci (DYNLT1 and AAS1) were measured. (FIG. 25B). Shown are absolute mKate knock-in efficiency as measured by mKate+ cell percentage using HEK293 human cells, each experiment was performed in triplicate (n=3), where the negative control is without recombinator (NR).

FIGS. 26A and 26B are the measurements of precision recombineering activity via mKate knock-in gene-editing assay using RecE and RecT homologs at the DYNLT1 locus (A) and the HSP90AA1 locus (B). Shown are absolute mKate knock-in efficiency as measured by mKate+ cell percentage using HEK293 human cells, each experiments were performed in triplicate (n=3), where the negative control is without recombinator (NR) and no cutting (NC). The original RecE and RecT from E. coli were also included as positive controls.

FIGS. 27A and 27B is a schematic showing the SunTag-based recruitment of SSAP RecT to Cas9-gRNA complex for gene-editing (FIG. 27A) and a graph quantifying the editing efficiencies of SunTag compared to MS2-based strategies (FIG. 27B).

FIGS. 28A-28C show comparisons of REDIT with alternative HDR-enhancing gene-editing approaches. FIG. 28A is schematics showing alternative HDR-enhancing approaches via fusing functional domains, CtIP or Geminin (Gem), to Cas9 protein (left) and when combined with REDIT (right). FIG. 28B is an alternative small-molecule HDR-enhancing approach through cell cycle control. Nocodazole was used to synchronize cells at the G2/M boundary (left) according to the timeline shown (right). FIG. 28C is comparisons of gene-editing efficiencies using REDIT and alternative HDR-enhancing tools, Cas9-HE (CtIP fusion), Cas9-Gem (Geminin fusion), and Nocodazole (noc), along with combination of REDIT with these methods (Cas9-HE/Cas9-Gem/noc+REDIT). Donor DNAs have 200+400 bp (DYNLT1) or 200+200 bp (HSP90AA1) of HAs. All assays performed with no donor, NTC and Cas9 (no enhancement) controls. #P<0.05, compared to REDIT; ## P<0.01, compared to REDIT.

FIGS. 29A-29D show template design guideline, junction precision, and capacity of REDIT gene-editing methods. FIG. 29A is graphs of a homology arm (HA) length test comparing different template designs of HDR donors (longer HAs) or NHEJ/MMEJ donors (zero/shorter HAs) using REDIT and Cas9 references. Top and bottom are two genomic loci tested using mKate knock-in assay. FIG. 29B is a design of an exemplary junction profiling assay through isolation of knock-in clones, followed by genomic PCR using primers (fwd, rev) binding outside donor to avoid template amplification. Paired Sanger sequencing of the PCR products reveal homologous and non-homologous edits at 5′- and 3′-junctions. FIG. 29C is a graph of the percentage of colonies with indicated junction profiles from the Sanger sequencing of knock-in clones as in FIG. 29B. Editing methods and donor DNA are listed at the bottom (HA lengths indicated in bracket). FIG. 29D is a graph of knock-in efficiencies using a 2-kb cassette to insert dual-GFP/mKate tags to validate REDIT methods with Cas9. HA lengths of donor DNAs indicated at the bottom.

FIGS. 30A-30C show GISseq results (FIG. 6C-E) indicating that REDIT is an efficient method with the ability to insert kilobase-length sequences with less unwanted editing events. FIG. 30A is a schematic showing the design, procedures, and analysis steps for GIS-seq to measure genome-wide insertion sites of the knock-in cassettes. High-molecular-weight (HMW) genomic DNA purification was needed to remove potential contamination from donor DNAs. Donor DNAs had 200 bp HAs each side. FIG. 30B is representative GIS-seq results showing plus/minus reads at on-target locus DYNLT1. The expected 2A-mKate knock-in site before the stop codon of the last exon are the center of the trimmed reads (reads clipped to remove 2A-mKate cassette). The template mutations help to avoid gRNA targeting and distinguish genomic and edited reads are labeled. FIG. 30C is a summary of top GIS-seq insertion sites comparing Cas9dn and REDITdn groups, showing the expected on-target insertion site (highlighted) and reduced number of identified off-target insertion sites when using REDITdn. (Left) DYNLT1 and (Right) ACTB loci with MLE calculated from the distribution of filtered and trimmed GIS-seq reads.

FIGS. 31A-31F show the dependence of REDIT gene-editing on endogenous DNA repair and applying REDIT methods for human stem cell engineering. FIG. 31A is a model showing the editing process and major repair pathways involved when using REDIT or Cas9 for gene-editing, the HDR pathway are highlighted for chemical perturbation (inhibition of RAD51). Donor DNAs with 200+200 bp HAs are used for all inhibitor experiments. FIGS. 31B and 31C are graphs showing the relative knock-inefficiency of REDIT tools compared with Cas9 reference treated with RAD51 inhibitor B02 and RI-1, or vehicle-treated, for the wtCas9-based REDIT and Cas9 (FIG. 31B) and for Cas9 nickase-based REDITdn and Cas9dn (FIG. 31C). All conditions were measured with 1-kb knock-in assay at two genomic loci (DYNLT1 and HSP90AA1). FIG. 31D are graphs of knock-in efficiencies in hESCs (H9) using REDIT and REDITdn tested across three genomic loci, compared with corresponding Cas9 and Cas9dn references. FIGS. 31E and 31F are flow cytometry plots of mKate knock-in results in hESCs using REDIT, REDITdn with Cas9, Cas9dn, and NTC controls. Donor DNAs in the hESC experiments have 200+200 bp HAs across all loci tested.

FIGS. 32A-32B show chemical perturbations to dCas9 REDIT. Gene editing efficiencies were determined when treated with mammalian DNA repair pathway inhibitors (Mirin, RI-1, and B02) with (FIG. 32A) and without (FIG. 32B) cell cycle inhibitor (Thy, doubly Thymidine) blocking. Statistical analyses are from t-test results with 1% FDR via a two-stage step-up method.

FIGS. 33A and 33B are schematics of the DNA components (gene-editing vectors and template DNA) and tail vein injection of mice, respectively.

FIGS. 34A-34C are results from the tail vein injection of mice with gene-editing vectors. FIG. 34A is a schematic and gel electrophoresis of PCR analysis of liver hepatocytes from the injected mice. FIG. 34B is the Sanger sequencing results of the PCR amplicon. FIG. 34C is a schematic of next-generation sequencing and a graph of the quantification of knock-in junction errors.

FIGS. 35A and 35B are schematics of the DNA components (gene-editing and control vector) and adeno-associated virus (AAV) treatment, respectively. FIG. 35C are fluorescent images of lungs from AAV treated mice and graphs of corresponding quantitation of tumor number.

FIGS. 36A-36C show the predicted structure of E. coli RecT (EcRecT) alone (FIG. 36A) and with bound single-strand DNA (FIG. 36B, 36C).

FIGS. 37A-37B show predicted interactions of EcRecT SSAP amino acids with SSDNA.

FIGS. 38A-38F show development of the dCas9 gene-editor through mining microbial SSAPs. (FIG. 38A) Schematic model of dCas9 editor with single-strand annealing proteins (SSAP). (FIG. 38B) Design of the genomic knock-in assay to measure gene-editing efficiencies (left); workflow of the SSAP screening experiments (right). (FIG. 38C) Construct designs for screening gene-editing efficiency of SSAPs using the 2A-mKate knock-in assay, with an 800 bp transgene. (FIG. 38D) Results of initial screen of three SSAPs: Bet protein from Lambda phage (LBet), RecT protein from Rac prophage (RacRecT), and gp2.5 from T7 phage (T7gp2.5). (FIG. 38E) Screening RecT-like SSAP candidates via metagenomic homolog mining and knock-in assay. The most active candidate is labeled as dCas9-SSAP. NTC: non-target control. Donor templates were added in all groups except the no-donor controls, with the homology arm (HA) lengths: DYNLT1, 200+200 bp; HSP90AA1, 200+400 bp; ACTB, 200+400 bp. (FIG. 38F) Measure gene-editing efficiencies using three types of donor designs with different HA lengths at DYNLT1 (left) and HSP90AA1 (right) loci in HEK293T cells. All results in this and following figures are from replicate experiments with error bars representing standard error of the mean (S.E.M.), n=3, unless otherwise noted.

FIGS. 39A-39H show on-target and off-target editing errors of dCas9-SSAP. (FIG. 39A) Deep sequencing to measure the levels of indel formation when using dCas9-SSAP and Cas9 references at endogenous targets. The donor templates used are 200 bp-HA HDR templates. Details of the assay described in Methods. (FIG. 39B) Clonal Sanger sequencing to analyze the accuracy of knock-in editing using dCas9-SSAP and Cas9 references with different HDR and MMEJ donors. The donor templates used are the 200 bp-HA HDR templates and 25 bp-HA MMEJ templates (Methods and Supplementary Notes). (FIG. 39C-FIG. 39E) Genome-wide detection of insertion sites of knock-in cassette using unbiased sequencing, showing (FIG. 39C) workflow, (FIG. 39D) representative reads aligned at knock-in genomic site, and (e) summary of detected on-target and off-target insertion sites. (FIG. 39F-FIG. 39G) workflow and results for measuring cell fitness effect as defined by percentage of live cells after editing (normalized to mock controls). (FIG. 39H) Summary analysis of knock-in accuracy of dCas9-SSAP editor, in comparison with Cas9 HDR and Cas9 MMEJ methods. Accuracy is defined as the overall yield (%) of correct knock-in within all edited outcomes (correct knock-in, knock-in with indels, and NHEJ indels).

FIGS. 40A-40G show validation of dCas9-SSAP editor and comparison with Cas9 reference and other HDR-enhancing methods. (FIG. 40A) Comparison of efficiencies using dCas9-SSAP and other alternative Cas9, nCas9, and HDR-enhancing tools. Cas9-HE (CtIP-fusion Cas9), and Cas9-Gem (Geminin-fusion Cas9), nCas9 (Cas9-D10A nickase reference), and nCas9-hRAD51 (am improved Cas9 nickase editor). Donor templates are same as in FIG. 1. (FIG. 40B) Imaging verification of mKate knock-in at endogenous genome locus using dCas9-SSAP editor. (FIG. 40C) Design of knock-in donor with different lengths of transgenes. (FIG. 40D) knock-in efficiencies for different transgene lengths using dCas9-SSAP editors. Donor HA lengths are 200 bp+200 bp for DYNLT1, 200 bp+400 bp for HSP90AA1. (FIG. 40E) performance of dCas9-SSAP editor compared with Cas9 references across 7 endogenous loci in HEK293T cells. ND, no-donor controls; NT, non-target controls. (FIG. 40F-FIG. 40G) knock-in gene-editing in human embryonic stem cells (hESC, H9) using dCas9-SSAP editor, with quantified HDR efficiencies (FIG. 40F) and flow cytometry analysis (FIG. 40G). All statistical analysis are performed using multiple t-test to compare across all genomic targets, with 1% false-discovery rate (FDR) via a two-stage step-up method of Benjamini, Krieger and Yekutieli.

FIGS. 41A-41D show chemical perturbations to probe the editing mechanism of dCas9-SSAP editor. Gene-editing efficiency of dCas9-SSAP editor when treated with DNA repair pathway inhibitors (Mirin, RI1 and B02) without FIGS. 41A, 41B) or with (FIGS. 41C, 41D) cell cycle synchronization (DTB, double Thymidine blocking). All donor templates are the same as in FIG. 38. Statistical analysis are from t-test results with 1% FDR via a two-stage step-up method of Benjamini, Krieger and Yekutieli.

FIGS. 42A-42D show minimization of dCas9-SSAP editor as a compact CRISPR knock-in tool for convenient delivery. (FIG. 42A) Schematic showing the EcRecT predicted secondary structure and priming sites for constructing truncated EcRecT proteins based on the structural prediction. (FIG. 42B) Relative knock-in efficiencies of various truncated designs. All groups were normalized to Cas9 references (individually for each target). (FIG. 42C) Schematic of dSaCas9-mSSAP system in AAV construct using the compact SaCas9 (left, sizes of elements not shown to scale) and (FIG. 42D) knock-in efficiencies at AAVS1 and HSP90AA1 endogenous targets via in vitro delivery of AAV2 vectors carrying the original and minimized dSaCas9-SSAP editors in HEK293T cells.

FIGS. 43A-43E show gel electrophoresis and sequencing verification of knock-in-specific PCR products using dCas9-SSAP. (FIG. 43A) Agarose gel results of knock-in-specific junction PCR at DYNLT1 locus. (FIG. 43B-FIG. 43E) Sanger sequencing chromatogram of genomic junctions from knock-in experiments at DYNLT1 locus. For all samples, we amplified the 5′ (FIG. 43B, FIG. 43C) and 3′ (FIG. 43D, FIG. 43E) end of genomic DNA using junction-spanning primers outside of the donor DNAs to confirm knock-in.

FIG. 44 shows a phylogenetic tree and amino acid alignment of representative RecT homologs along with the protein conserved domain annotated.

FIGS. 45A-45B show deep sequencing of short-sequence editing comparing dCas9-SSAP and Cas9 editors. (FIG. 45A) Donor design of 16-bp replacement at EMX1. (FIG. 45B) Analysis of precision HDR and indel editing outcomes using deep sequencing at EMX1 genomic locus. The first round of PCR used sequencing primers completely outside of the donor to ensure the sequencing results will be free from the donor template contamination, validated by the non-target control (where the donor DNAs are delivered into the cells).

FIGS. 46A-46B are schematics showing the workflows used in Sanger sequencing of knock-in products (FIG. 46A) and the sequencing method used in deep on-target indel assay (FIG. 46B). Assays described here correspond to FIG. 41. gPCR, genomic PCR. Seq-F/seq-R are primers for Sanger sequencing binding upstream/downstream of the knock-in templates.

FIGS. 47A-47B show Sanger sequencing chromatograms of genomic junctions from dCas9-SSAP experiments at DYNLT1 locus. For all samples, 5′ (FIG. 47A) and 3′ (FIG. 47B) ends of genomic DNA were amplified using junction-spanning primers to confirm knock-in precision. The genomic-binding primers used are completely outside of the donor DNAs to avoid contamination.

FIGS. 48A-48B show Sanger sequencing chromatograms of genomic junctions from dCas9-SSAP experiments at HSP90AA1 locus. For all samples, 5′ (FIG. 48A) and 3′ (FIG. 48B) end of genomic DNA were amplified using junction-spanning primers to confirm knock-in precision. The genomic-binding primers used are completely outside of the donor DNAs to avoid contamination.

FIGS. 49A-49B show genome-wide insertion site mapping and quantification. (FIG. 49A) Overall workflow for unbiased genome-wide insertion site mapping process. On-target and off-target insertions sites are recovered from reads that align to the reference genome (hg38). Full protocol and data analysis pipeline are detailed in Methods. (FIG. 49B) Quantification of genome-wide insertion sites counting all aligned reads (with valid UMI) showed decreased insertion site abundance using Cas9-SSAP compared with Cas9 HDR, across two genomic loci (DYNLT1 and HSP90AA1). The abundance of insertion sites are measured as RPKU, or Reads Per Thousand UMIs.

FIGS. 50A-50B show testing of dCas9-SSAP editor tool using single-guide (FIG. 50A) and dual-guide (FIG. 50B) designs across three genomic targets (shown on the top). The donor DNAs used are the same as shown in FIG. 3a with 800-bp knock-in design.

FIGS. 51A-51C show validation of dCas9-SSAP knock-in efficiencies in three additional cell lines in HepG2 (FIG. 51A), HeLa (FIG. 51B), and U2OS (FIG. 51C) cell lines. The knock-in experiments used similar donor DNA with ˜800-bp cassettes encoding 2A-mKate transgene for all cell lines tested.

FIGS. 52A-52C show the full set of flow cytometry analysis data using dCas9-SSAP editor for human stem cell engineering. Flow cytometry analysis of knock-in gene-editing at HSP90AA1 (FIG. 52A), ACTB (FIG. 52B), OCT4 (FIG. 52C) endogenous loci in human embryonic stem cells (hESC, H9) using dCas9-SSAP compared with non-target controls and Cas9 (Cas9 HDR) references.

FIG. 53 is a schematic showing the RecT protein secondary structure predicted using an online tool (CFSSP, see Methods). The prediction results (secondary structure visualized at top, alignment at bottom) formed the basis for developing a truncated functional RecT variant.

FIG. 54 depicts SSAP array screening, showing cell viability vs. editing efficiency (fold over negative control (A, C) or percent of mKate knock-in (B, D)) for the ACTB target (A, B) and the HSP90AA1 target (C. D). The positive control is EcRecT.

FIG. 55 depicts normalized (A) and absolute (B) editing efficiency, comparing activity at two targets, HSP90AA and ACTB. FIG. 55C shows cell viability, comparing SSAP use for HSP90AA1 knock-ins with ACTB knock-ins. The positive control is EcRecT.

FIG. 56 depicts by scatter plot a comparison of cell viability vs. normalized (A) or absolute (B) editing efficiency for all targets combined. Bar graphs compare editing efficiency at two targets, HSP90 and QCTB, normalized (C) or absolute (D) for each of the candidates. The positive control is EcRecT.

FIG. 57 depicts a tree and sequence alignment for SSAP_16 (1, SEQ ID NO:185), SSAP_10 (2, SEQ ID NO: 179), SSAP_36 (3, SEQ ID NO:205), SSAP_152 (4, SEQ ID NO:321), and SSAP_184 (5, SEQ ID NO:353) compared with EcRecT (SEQ ID NO:171). See Table 9.

FIG. 58 depicts a tree and sequence alignment for SSAP_16 (1, SEQ ID NO:185), SSAP_10 (2, SEQ ID NO:179), SSAP_36 (3, SEQ ID NO:205), SSAP_152 (4, SEQ ID NO:321), SSAP_184 (5, SEQ ID NO:353), SSAP_197 (6, SEQ ID NO:366), SSAP_305 (7, SEQ ID NO: 424), SSAP_210 (8; SEQ ID NO:379), and SSAP_190 (9, SEQ ID NO:359) compared with EcRecT (SEQ ID NO:171). See Table 9.

FIG. 58 depicts a tree and sequence alignment for SSAP_16 (1, SEQ ID NO:185), SSAP_10 (2, SEQ ID NO: 179), SSAP_36 (3, SEQ ID NO:205), SSAP_197 (6, SEQ ID NO:366), and SSAP_210 (8; SEQ ID NO:379) compared with EcRecT (SEQ ID NO: 171). See Table 9.

### DETAILED DESCRIPTION OF THE INVENTION

Current genome editing technology is limited by the low efficiency and accuracy for precision editing leading to very unreliable ability for using current tools such as CRISPR systems to introduce accurate replacement, deletion, or insertion in mammalian cells. The usual process involves delivery of gene editing tool (like CRISPR) and DNA repair template for introducing desirable changes to genome sequence. However, the DNA delivered into the cell can insert non-specifically into off-target genomic loci or unintended targets, a major challenge for ensuring safe, accurate gene editing for therapeutic purposes.

The present disclosure is directed to a system and the components for DNA editing. In particular, the disclosed system based on CRISPR targeting and homology directed repair by phage recombination enzymes. The system results in superior recombination efficiency and accuracy at a kilobase scale.

The invention features RNA as a molecular entity to mediate gene editing, and includes designed and validated components of systems and methods to apply RNA as template (donor) to insert, delete, replace, or control genomic DNA sequences, mediated through the activity of a recombination protein such as a SSAP (single-strand annealing protein, exemplified by RccT, lambda Red, T7gp2.5).

In certain embodiments, the invention provides efficient gene editing through the process of delivering three components into a cell: (1) local DNA cleavage, nicking, or R-loop-formation using the CRISPR system comprising a CRISPR enzyme (including but not limited to Cas9/Cas9n/dCas9 or Cas12a/nCas12a/dCas12a respectively for cleavage/nick/R-loop-formation), and a guide RNA, where the guide RNA contains an aptamer (such as MS2, or PP7, or BoxB) to recruit SSAP protein; (2) an RNA sequence bearing the desirable DNA changes with one or more homology arm (HA) region(s) that is either fused/linked to the guide RNA in (1), or fused/linked to a second guide RNA. The HA region is at least 20 bp and provides a homology region next to the editing site for SSAP-mediated editing. If using a second guide RNA, this second guide RNA will bind to a nearby genomic site, located between 0 bp to 150 bp away from the guide RNA in (1). This second guide RNA forms a complex with a CRISPR enzyme (such as Cas9/nCas9/dCas9 and Cas12a/nCas12a/dCas12a), is recruited to the target genomic loci, and serves to provide RNA template/donor for the editing. The enzymes can be either fully active CRISPR enzymes, nickases, or deactivated CRISPR enzymes (dCas9, dCas12a, etc.) that only bind to target loci. The guide may be regular guide RNA or shorter guide RNA (typically 2˜6 bp shorter than the regular guide RNA, so 14 bp to 18 bp) to allow efficient binding but not cleavage of targets. (3) SSAP protein fused to an RNA-aptamer-binding protein (RBP) via linker. The RBP can be, without limitation, MS2 coat protein (MCP), PP7 coat protein (PCP), or BoxB binding peptide from lambda phage (lambda N22 peptide). For this component, we also identified an additional factor that could enhance this RNA-templated SSAP gene-editing: if we fuse a reverse transcriptase (RT) to the SSAP protein via a long peptide linker, making this third component RBP-SSAP-RT, or RBP-RT-SSAP (-represent linkers), this further enhance editing efficiencies.

In other embodiments, the Cas9/nCas9/dCas9 or Cas12a/nCas12a/dCas12a protein is fused via linker to a reverse transcriptase (RT), this design is comparable to the prime-editing. The guide RNA in this design optionally comprises a primer-binding-site (PBS) of at least 14-bp or more, which is complementary to a region at the editing site. This PBS promotes initiation of RT activity. Alternatively, another design is to use the same guide RNA as in the first embodiment, and to initiate RT activity by supplying to the cell a short oligo DNA (length is 14 bp or more) that is complementary to a region at the editing site. This oligo DNA can initiate RT activity and allow SSAP-mediated gene-editing.

In other embodiments, the Cas9/nCas9/dCas9 or Cas12a/nCas12a/dCas12a protein is fused via linker to a reverse transcriptase (RT) from a retron system. The guide RNA in this design has a msr/msd sequence from retron, and also one or more homology arm (HA) region(s), which is complementary to a region at the editing site. The msr/msd sequence helps to initiate RT activity. While the HA region help to mediate SSAP gene-editing.

The compositions and methods of the invention herein provide novel RNA-mediated/RNA-templated gene editing in eukaryotic/mammalian cells. By designing cleavable RNA template using endogenous tRNA, ribozyme, or the direct repeat from Cas 12a system, we also achieve multiple-target gene editing using RNA as template.

The invention provides at least the following 5 advantages of our RNA-templated SSAP gene editing system: (1) reduced off-target or toxicity due to RNA being less immunogenic compared with DNA used in existing gene editing process, and that RNA cannot integrated directly into unintended genomic DNA sites or off-target DNA sites; (2) case of multiplexing the precision gene editing methods by using cleavable RNA template in our methods; (3) simplicity of RNA delivery into cells, it is easier to manufacture, potentially cheaper to scale up for clinical usage (4) RNA has a lot of engineering potential by combining other regulatory or combinatorial payload/components via chemical linkage or biochemical coupling, to enable more efficiency delivery, editing, or synergistic action of RNA-templated gene editing with other type of gene editing or therapeutic modalities; and (5) the efficiency of RNA-templated gene editing can be enhanced via RNA and protein factors and is orthogonal to regular DNA-repair pathways that may be critical for health of target cells.

### 1. Definitions

To facilitate an understanding of the present technology, a number of terms and phrases are defined below. Additional definitions are set forth throughout the detailed description.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The terms “complementary” and “complementarity” refer to the ability of a nucleic acid to form hydrogen bond(s) with another nucleic acid sequence by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., 50%, 60%, 70%, 80%, 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., 65%, 70%, 75%, 80%, 85%, 90%, 95%. 97%, 98%, 99%, or 100%) over a region of at least 8 nucleotides (e.g., 9, 10, 11, 12, 13, 14, 15, 16, 17, 18, 19, 20, 21, 22, 23, 24, 25, 30, 35, 40, 45, 50, or more nucleotides), or if the two nucleic acid sequences hybridize under at least moderate, preferably high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., infra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (preferably in combination with EDTA). Additional details and an explanation of stringency of hybridization reactions are provided in, e.g., Sambrook et al., Molecular Cloning: A Laboratory Manual, 3rd ed., Cold Spring Harbor Press, Cold Spring Harbor, N.Y. (2001); and Ausubel et al., Current Protocols in Molecular Biology, Greene Publishing Associates and John Wiley & Sons, New York (1994).

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

As used herein, a “nucleic acid” or a “nucleic acid sequence” refers to a polymer or oligomer of pyrimidine and/or purine bases, preferably cytosine, thymine, and uracil, and adenine and guanine, respectively. The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (scc, e.g., Braasch and Corey, Biochemistry, 41 (14): 4503-4510 (2002)) and U.S. Pat. No. 5,034,506, incorporated herein by reference), locked nucleic acid (LNA; see Wahlestedt et al., Proc. Natl. Acad. Sci. U.S.A., 97:5633-5638 (2000), incorporated herein by reference), cyclohexenyl nucleic acids (scc Wang, J. Am. Chem. Soc., 122:8595-8602 (2000), incorporated herein by reference), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein,” are used interchangeably herein.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

The term “percent sequence similarity” takes into account conservative amino acid substitutions. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions, or additions which alter, add, or delete a single amino acid or a small percentage of amino acids in an encoded sequence are also “conservatively modified variations.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

It will be understood that sequences identified as having greater than a given percent similarity to a reference sequence include as a subset the sequences having greater than the given percent identity to the reference sequence. Thus, recitations herein to sequences having greater than a given percent similarity include the subset of sequences having greater than a given percent identity.

A “vector” or “expression vector” is a replicon, such as plasmid, phage, virus, or cosmid, to which another DNA segment, e.g., an “insert,” may be attached or incorporated so as to bring about the replication of the attached segment in a cell.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (e.g., altered characteristics) when compared to the wild-type gene or gene product. It is noted that naturally occurring mutants can be isolated; these are identified by the fact that they have altered characteristics when compared to the wild-type gene or gene product.

### 2. RNA-Guided CRISPR Recombineering System

In bacteria and archaea, CRISPR/Cas systems provide immunity by incorporating fragments of invading phage, virus, and plasmid DNA into CRISPR loci and using corresponding CRISPR RNAs (“crRNAs”) to guide the degradation of homologous sequences. Each CRISPR locus encodes acquired “spacers” that are separated by repeat sequences. Transcription of a CRISPR locus produces a “pre-crRNA,” which is processed to yield crRNAs containing spacer-repeat fragments that guide effector nuclease complexes to cleave dsDNA sequences complementary to the spacer. Three different types of CRISPR systems are known, type I, type II, or type III, and classified based on the Cas protein type and the use of a proto-spacer-adjacent motif (PAM) for selection of proto-spacers in invading DNA. The endogenous type II systems comprise the Cas9 protein and two noncoding crRNAs: trans-activating crRNA (tracrRNA) and a precursor crRNA (pre-crRNA) array containing nuclease guide sequences (also referred to as “spacers”) interspaced by identical direct repeats (DRs). tracrRNA is important for processing the pre-crRNA and formation of the Cas9 complex. First, tracrRNAs hybridize to repeat regions of the pre-crRNA. Second, endogenous RNaseIII cleaves the hybridized crRNA-tracrRNAs, and a second event removes 5′ end of each spacer, yielding mature crRNAs that remain associated with both the tracrRNA and Cas9. Third, each mature complex locates a target double stranded DNA (dsDNA) sequence and cleaves both strands using the nuclease activity of Cas9.

CRISPR/Cas gene editing systems have been developed to enable targeted modifications to a specific gene of interest in eukaryotic cells. CRISPR/Cas gene editing systems are commonly based on the RNA-guided Cas9 nuclease from the type II prokaryotic clustered regularly interspaced short palindromic repeats (CRISPR) adaptive immune system. Engineering CRISPR/Cas systems for use in eukaryotic cells typically involves reconstitution of the crRNA-tracrRNA-Cas9 complex. In human cells, for example, the Cas9 amino acid sequence may be codon-optimized and modified to include an appropriate nuclear localization signal, and the crRNA and tracrRNA sequences may be expressed individually or as a single chimeric molecule via an RNA polymerase II promoter. Typically, the crRNA and tracrRNA sequences are expressed as a chimera and are referred to collectively as “guide RNA” (gRNA) or single guide RNA (sgRNA). Thus, the terms “guide RNA,” “single guide RNA,” and “synthetic guide RNA,” are used interchangeably herein and refer to a nucleic acid sequence comprising a tracrRNA and a pre-crRNA array containing a guide sequence. The terms “guide sequence,” “guide,” and “spacer,” arc used interchangeably herein and refer to the about 20 nucleotide sequence within a guide RNA that specifies the target site. In CRISPR/Cas9 systems, the guide RNA contains an approximate 20-nucleotide guide sequence followed by a protospacer adjacent motif (PAM) that directs Cas9 via Watson-Crick base pairing to a target sequence.

In some embodiments, the disclosure provides a system for RNA-guided recombineering utilizing tools from CRISPR gene editing systems. The system comprises: a Cas protein, a nucleic acid molecule comprising a guide RNA sequence that is complementary to a target DNA sequence and a recombination protein. In certain embodiments, the recombination protein comprises a microbial recombination protein. In certain embodiments, the recombination protein comprises a viral recombination protein. In certain embodiments, the recombination protein comprises a eukaryotic recombination protein. In certain embodiments, the recombination protein comprises a mitochondrial recombination protein.

Cas protein families are described in further detail in, e.g., Haft et al., PLOS Comput. Biol., 1 (6): c60 (2005), incorporated herein by reference. The Cas protein may be any Cas endonucleases. In some embodiments, the Cas protein is Cas9 or Cas 12a, otherwise referred to as Cpf1. In one embodiment, the Cas9 protein is a wild-type Cas9 protein. The Cas9 protein can be obtained from any suitable microorganism, and a number of bacteria express Cas9 protein orthologs or variants. In some embodiments, the Cas9 is from Streptococcus pyogenes or Staphylococcus aureus. Cas9 proteins of other species are known in the art (see, e.g., U.S. Patent Application Publication 2017/0051312, incorporated herein by reference) and may be used in connection with the present disclosure. The amino acid sequences of Cas proteins from a variety of species are publicly available through the GenBank and UniProt databases.

In some embodiments, the Cas9 protein is a Cas9 nickase (Cas9n). Wild-type Cas9 has two catalytic nuclease domains facilitating double-stranded DNA breaks. A Cas9 nickase protein is typically engineered through inactivating point mutation(s) in one of the catalytic nuclease domains causing Cas9 to nick or enzymatically break only one of the two DNA strands using the remaining active nuclease domain. Cas9 nickases are known in the art (see, e.g., U.S. Patent Application Publication 2017/0051312, incorporated herein by reference) and include, for example, Streptococcus pyogenes with point mutations at D10 or H840. In select embodiments, the Cas9 nickase is Streptococcus pyogenes Cas9n (D10A).

In some embodiments, the Cas protein is a catalytically dead Cas. For example, catalytically dead Cas9 is essentially a DNA-binding protein due to, typically, two or more mutations within its catalytic nuclease domains which renders the protein with very little or no catalytic nuclease activity. Streptococcus pyogenes Cas9 may be rendered catalytically dead by mutations of D10 and at least one of E762, H840, N854, N863, or D986, typically H840 and/or N863 (see, e.g., U.S. Patent Application Publication 2017/0051312, incorporated herein by reference). Mutations in corresponding orthologs are known, such as N580 in Staphylococcus aureus Cas9. Oftentimes, such mutations cause catalytically dead Cas proteins to possess no more than 3% of the normal nuclease activity.

In some embodiments, the system comprises a nucleic acid molecule comprising a guide RNA sequence complementary to a target DNA sequence. The guide RNA sequence, as described above, specifies the target site with an approximate 20-nucleotide guide sequence followed by a protospacer adjacent motif (PAM) that directs Cas9 via Watson-Crick base pairing to a target sequence.

The terms “target DNA sequence,” “target nucleic acid,” “target sequence,” and “target site” are used interchangeably herein to refer to a polynucleotide (nucleic acid, gene, chromosome, genome, etc.) to which a guide sequence (e.g., a guide RNA) is designed to have complementarity, wherein hybridization between the target sequence and a guide sequence promotes the formation of a Cas9/CRISPR complex, provided sufficient conditions for binding exist. In some embodiments, the target sequence is a genomic DNA sequence. The term “genomic,” as used herein, refers to a nucleic acid sequence (e.g., a gene or locus) that is located on a chromosome in a cell. The target sequence and guide sequence need not exhibit complete complementarity, provided that there is sufficient complementarity to cause hybridization and promote formation of a CRISPR complex. A target sequence may comprise any polynucleotide, such as DNA or RNA. Suitable DNA/RNA binding conditions include physiological conditions normally present in a cell. Other suitable DNA/RNA binding conditions (e.g., conditions in a cell-free system) are known in the art; see, e.g., Sambrook, referenced herein and incorporated by reference. The strand of the target DNA that is complementary to and hybridizes with the DNA-targeting RNA is referred to as the “complementary strand” and the strand of the target DNA that is complementary to the “complementary strand” (and is therefore not complementary to the DNA-targeting RNA) is referred to as the “noncomplementary strand” or “non-complementary strand.”

The target genomic DNA sequence may encode a gene product. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target genomic DNA sequence encodes a protein or polypeptide.

In some embodiments, for instance, when the system includes a Cas9 nickase or a catalytically dead Cas 9, two nucleic acid molecules comprising a guide RNA sequence may be utilized. The two nucleic acid molecules may have the same or different guide RNA sequences, thus complementary to the same or different target DNA sequence. In some embodiments, the guide RNA sequences of the two nucleic acid molecules are complementary to a target DNA sequences at opposite ends (e.g., 3′ or 5′) and/or on opposite strands of the insert location.

In some embodiments, the system further comprises a recruitment system comprising at least one aptamer sequence and an aptamer binding protein functionally linked to the recombination protein as part of a fusion protein.

In some embodiments, the aptamer sequence is an RNA aptamer sequence. In some embodiments, the nucleic acid molecule comprising the guide RNA also comprises one or more RNA aptamers, or distinct RNA secondary structures or sequences that can recruit and bind another molecular species, an adaptor molecule, such as a nucleic acid or protein. Several CRISPR systems are compatible with guide RNA insertions and extensions, including but not limited to SpCas9, SaCas9, and LbCas12a (aka Cpf1). The RNA aptamers can be naturally occurring or synthetic oligonucleotides that have been engineered through repeated rounds of in vitro selection or SELEX (systematic evolution of ligands by exponential enrichment) to bind to a specific target molecular species. In some embodiments, the nucleic acid comprises two or more aptamer sequences. The aptamer sequences may be the same or different and may target the same or different adaptor proteins. In select embodiments, the nucleic acid comprises two aptamer sequences.

Any RNA aptamer/aptamer binding protein pair known may be selected and used in connection with the present disclosure (see, e.g., Jayasena, S. D., Clinical Chemistry, 1999. 45 (9): p. 1628-1650; Gelinas, et al., Current Opinion in Structural Biology, 2016. 36: p. 122-132; and Hasegawa, H., Molecules, 2016; 21 (4): p. 421, incorporated herein by reference).

A number of RNA aptamer binding, or adaptor, proteins exist, including a diverse array of bacteriophage coat proteins. Examples of such coat proteins include but are not limited to: MS2, QB, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. In some embodiments, the RNA aptamer binds MS2 bacteriophage coat protein or a functional derivative, fragment, or variant thereof. MS2 binding RNA aptamers commonly have a simple stem-loop structure, classically defined by a 19 nucleotide RNA molecule with a single bulged adenine on 5′ leg of the stem (Witherall G. W., et al., (1991) Prog. Nucleic Acid Res. Mol. Biol., 40, 185-220, incorporated herein by reference). However, a number of vastly different primary sequences were found to be able to bind the MS2 coat protein (Parrott A M, et al., Nucleic Acids Res. 2000; 28 (2): 489-497, Buenrostro J D, et al. Natura Biotechnology 2014; 32, 562-568, and incorporated herein by reference). Any of the RNA aptamer sequence known to bind the MS2 bacteriophage coat protein may be utilized in connection with the present disclosure to bind to fusion proteins comprising MS2. In select embodiments, the MS2 RNA aptamer sequence comprises: AACAUGAGGAUCACCCAUGUCUGCAG (SEQ ID NO:145), AGCAUGAGGAUCACCCAUGUCUGCAG (SEQ ID NO:146), or AGCGUGAGGAUCACCCAUGCCUGCAG (SEQ ID NO:147).

N-proteins (Nut-utilization site proteins) of bacteriophages contain arginine-rich conserved RNA recognition motifs of ˜20 amino acids, referred to as N peptides. The RNA aptamer may bind a phage N peptide or a functional derivative, fragment, or variant thereof. In some embodiments, the phage N peptide is the lambda or P22 phage N peptide or a functional derivative, fragment, or variant thereof.

In select embodiments, the N peptide is lambda phage N22 peptide, or a functional derivative, fragment, or variant thereof. In some embodiments, the N22 peptide comprises an amino acid sequence with at least 70% similarity to the amino acid sequence GNARTRRRERRAEKQAQWKAAN (SEQ ID NO:149). N22 peptide, the 22 amino acid RNA-binding domain of the 2 bacteriophage antiterminator protein N (AN-(1-22) or AN peptide), is capable of specifically binding to specific stem-loop structures, including but not limited to the BoxB stem-loop. See, for example Cilley and Williamson, RNA 1997; 3 (1): 57-67, incorporated herein by reference. A number of different BoxB stem-loop primary sequences are known to bind the N22 peptide and any of those may be utilized in connection with the present disclosure. In some embodiments, the N22 peptide RNA aptamer sequence comprises a nucleotide sequence with at least 70% similarity to an RNA sequence selected from the group consisting of GCCCUGAAAAAGGGC (SEQ ID NO:150), GCCCUGAAGAAGGGC (SEQ ID NO:151), GCGCUGAAAAAGCGC (SEQ ID NO:152), GCCCUGACAAAGGGC (SEQ ID NO:153), and GCGCUGACAAAGCGC (SEQ ID NO:154). In some embodiments, the N22 peptide RNA aptamer sequence is selected from the group consisting of SEQ ID NOs: 150-154.

In select embodiments, the N peptide is the P22 phage N peptide, or a functional derivative, fragment, or variant thereof. A number of different BoxB stem-loop primary sequences are known to bind the P22 phage N peptide and variants thereof and any of those may be utilized in connection with the present disclosure. See, for example Cocozaki, Ghattas, and Smith, Journal of Bacteriology 2008; 190 (23): 7699-7708, incorporated herein by reference. In some embodiments, the P22 phage N peptide comprises an amino acid sequence with at least 70% similarity to the amino acid sequence GNAKTRRHERRRKLAIERDTI (SEQ ID NO:155). In some embodiments, the P22 phage N peptide RNA aptamer sequence comprises a sequence with at least 70% similarity to an RNA sequence selected from the group consisting of GCGCUGACAAAGCGC (SEQ ID NO:156) and CCGCCGACAACGCGG (SEQ ID NO:157).

In some embodiments, the P22 phage N peptide RNA aptamer sequence is selected from the group consisting of SEQ ID NOs: 156-157, UGCGCUGACAAAGCGCG (SEQ ID NO:158) or ACCGCCGACAACGCGGU (SEQ ID NO:159).

In certain embodiments, different aptamer/aptamer binding protein pairs can be selected to bring together a combination of recombination proteins and functions.

In some embodiments, the aptamer sequence is a peptide aptamer sequence. The peptide aptamers can be naturally occurring or synthetic peptides that are specifically recognized by an affinity agent. Such aptamers include, but are not limited to, a c-Myc affinity tag, an HA affinity tag, a His affinity tag, an S affinity tag, a methionine-His affinity tag, an RGD-His affinity tag, a 7×His tag, a FLAG octapeptide, a strep tag or strep tag II, a V5 tag, or a VSV-G epitopc. Corresponding aptamer binding proteins are well-known in the art and include, for example, primary antibodies, biotin, affimers, single domain antibodies, and antibody mimetics.

An exemplary peptide aptamer includes a GCN4 peptide (Tanenbaum et al., Cell 2014; 159 (3): 635-646, incorporated herein by reference). Antibodies, or GCN4 binding protein can be used as the aptamer binding proteins.

In some embodiments, the peptide aptamer sequence is conjugated to the Cas protein. The peptide aptamer sequence may be fused to the Cas in any orientation (e.g., N-terminus to C-terminus, C-terminus to N-terminus, N-terminus to N-terminus). In select embodiments, the peptide aptamer is fused to the C-terminus of the Cas protein.

In some embodiments, between 1 and 24 peptide aptamer sequences may be conjugated to the Cas protein. The aptamer sequences may be the same or different and may target the same or different aptamer binding proteins. In select embodiments, 1 to 24 tandem repeats of the same peptide aptamer sequence are conjugated to the Cas protein. In preferred embodiments between 4 and 18 tandem repeats are conjugated to the Cas protein. The individual aptamers may be separated by a linker region. Suitable linker regions are known in the art. The linker may be flexible or configured to allow the binding of affinity agents to adjacent aptamers without or with decreased steric hindrance. The linker sequences may provide an unstructured or linear region of the polypeptide, for example, with the inclusion of one or more glycine and/or serine residues. The linker sequences can be at least about 2, 3, 4, 5, 6, 7, 8, 9, 10 or more amino acids in length.

In some embodiments, the fusion protein comprises a recombination protein functionally linked to an aptamer binding protein. In some embodiments, the recombination protein comprises a microbial recombination protein. In some embodiments, the recombination protein comprises a recombinase. In certain embodiments, the recombination protein comprises 5′-3′ exonuclease activity. In certain embodiments, the recombination protein comprises 3′-5′ exonuclease activity. In certain embodiments, the recombination protein comprises ssDNA binding activity. In certain embodiments, the recombination protein comprises ssDNA annealing activity.

The bacteriophage A-encoded genetic recombination machinery, named the λ red system, comprises the exo and bet genes, assisted by the gam gene, together designated λ red genes. Exo is a 5′-3′ exonuclease which targets dsDNA and Bet is a ssDNA-binding protein. Bet functions include protecting ssDNA from degradation and promoting annealing of complementary ssDNA strands. Another bacteriophage system found in E. coli is the Rac prophage system, comprising recE and recT genes which are functionally similar to exo and bet. In some embodiments, the microbial recombination protein may be RecE, RecT, lambda exonuclease (Exo), Bet protein (betA, redB), exonuclease gp6, single-stranded DNA-binding protein gp2.5, or a derivative or variant thereof.

Recombination proteins and functional fragments thereof useful in the invention include nucleases, ssDNA-binding proteins (SSBs), and ssDNA annealing proteins (SSABs). Among microbial proteins, these include, without limitation, E. coli proteins such as ExoI (xonA; sbcB), ExoIII (xthA), ExoIV (orn), ExoVII (xseA, xseB), ExoIX (ygdG), ExoX (exoX), DNA poll 5′ Exo (ExoVI) (polA), DNA Pol I 3′ Exo (ExoII) (polA), DNA Pol II 3′ Exo (polB), DNA Pol III 3′ Exo (dnaQ, mutD), RecBCD (recB, recC, recD), and RecJ (recJ) and their functional fragments.

While double-stranded DNA contains genetic information, use of the information involves single-stranded intermediates. Whereas the single-stranded intermediates form secondary structures and are sensitive to chemical and nucleolytic degradation, cells encode ssDNA binding proteins (SSBs) that bind to and stabilize ssDNA. Useful SSBs include, without limitation, SSBs of prokaryotes, bacteriophage, eukaryotes, mammals, mitochondria, and viruses. While SSBs are found in every organism, the proteins themselves share surprisingly little sequence similarity, and may differ in subunit composition and oligomerization states. SSB proteins may comprise certain structural features. One is use of oligonucleotide/oligosaccharide-binding (OB) domains to bind ssDNA through a combination of electrostatic and base-stacking interactions with the phosphodiester backbone and nucleotide bases. Another feature is oligomerization that brings together DNA-binding OB folds. Eukaryotic SSBs are regulated by phosphorylation on serine and threonine residues. Tyrosine phosphorylation of microbial SSBs is observed in taxonomically distant bacteria and substantially increases affinity for ssDNA. The human mitochondrial ssDNA-binding protein is structurally similar to SSB from Escherichia coli (EcoSSB), but lacks the C-terminal disordered domain. Eukaryotic replication protein A (RPA) shares function, but not sequence homology with bacterial SSB. The herpes simplex virus (HSV-1) SSB, ICP8, is a nuclear protein that, along other replication proteins is required for viral DNA replication.

Without being bound by theory, it is thought that exonuclease activities and ssDNA binding activities of the recombination proteins of the invention uncover and protect single stranded regions of template and target DNAs, thereby facilitating recombination. Also, targeting can be cooperative, involving target directed CRISPR-mediated nicking of chromosomal DNA coordinated with recombination directed by homology arms designed into template DNAs. In certain embodiments of the invention, off-target effects are minimized. For example, whereas targeted recombination involves coordinated CRISPR and recombination functions, at off-target sites, homology with the HR template DNA is absent and nick repair may be favored.

Single stranded DNA annealing proteins (SSAPs) also are ubiquitous among organisms with diverse sequences and have been classified into families and superfamilies by bioinformatics and experimental analysis. Moreover, phage encoded SSAPs are recognized to encode their own SSAP recombinases which substitute for classic RecA proteins while functioning with host proteins to control DNA metabolism. Steczkiewiz classified SSAPs into seven families (RecA, Gp2.5, RecT/RedB, Erf, Rad52/22, Sak3, and Sak4) organized into three superfamilies including prokaryotes, eukaryotes, and phage (Steczkiewicz et al., 2021, Front. Microbiol 12:644622). Non-limiting examples of SSAPs that can be used according to the invention are provided in Table 5. Any one or more of the SSAPs can be employed in the invention.

In certain embodiments, a microbial recombination protein is RecE or RecT, or a derivative or variant thereof. Derivatives or variants of RecE and RecT are functionally equivalent proteins or polypeptides which possess substantially similar function to wild type RecE and RecT. RecE and RecT derivatives or variants include biologically active amino acid sequences similar to the wild-type sequences but differing due to amino acid substitutions, additions, deletions, truncations, post-translational modifications, or other modifications. In some embodiments, the derivatives may improve translation, purification, biological half-life, activity, or eliminate or lessen any undesirable side effects or reactions. The derivatives or variants may be naturally occurring polypeptides, synthetic or chemically synthesized polypeptides or genetically engineered peptide polypeptides. RecE and RecT bioactivities are known to, and easily assayed by, those of ordinary skill in the art, and include, for example exonuclease and single-stranded nucleic acid binding, respectively.

The RecE or RecT may be from a number of microbial organisms, including Escherichia coli, Pantoea breeneri, Type-F symbiont of Plautia stali, Providencia sp. MGF014, Shigella sonnei, Pseudobacteriovorax antillogorgiicola, among others. Other non-limiting sources include Desulfotalea psychrophila, Lactococcus lactis, Flavobacterium psychrophilum, Mycobacterium smegmatis, Lactobacillus rhamnosus, Psychrobacter arcticus, Psychrobacter cryohalolentis, Psychromonas ingrahamii, Photobacterium profundum, Psychroflexus torquis, and Caulobacter crescentus. In certain embodiments, the RecE and RecT protein is derived from Escherichia coli.

In some embodiments, the fusion protein comprises RecE, or a derivative or variant thereof. The RecE, or derivative or variant thereof, may comprise an amino acid sequence selected from the group consisting of SEQ ID NOs: 1-8. The RecE, or derivative or variant thereof, may comprise an amino acid sequences with at least 70% (e.g., 75%, 80%, 85%, 90%, 91%, 92%, 93%, 94%, 95%, 96%, 97%, 98%, 99%, or 100%) similarity to amino acid sequences selected from the group consisting of SEQ ID NOs: 1-8. In select embodiments, the RecE, or derivative or variant thereof, comprises an amino acid sequences with at least 90% similarity to amino acid sequences selected from the group consisting of SEQ ID NOs: 1-8. In exemplary embodiments, the RecE, or derivative or variant thereof, comprises an amino acid sequences with at least 90% similarity to amino acid sequences selected from the group consisting of SEQ ID NOs: 1-3.

In some embodiments, the fusion protein comprises RecT, or a derivative or variant thereof. The RecT, or derivative or variant thereof, may comprise an amino acid sequence selected from the group consisting of SEQ ID NOs: 9-14. The RecT, or derivative or variant thereof, may comprise an amino acid sequences with at least 70% (e.g., 75%, 80%, 85%, 90%, 91%, 92%, 93%, 94%, 95%, 96%, 97%, 98%, 99%, or 100%) similarity to amino acid sequences selected from the group consisting of SEQ ID NOs: 9-14. In select embodiments, the RecT, or derivative or variant thereof, comprises an amino acid sequences with at least 90% similarity to amino acid sequences selected from the group consisting of SEQ ID NOs: 9-14. In exemplary embodiments, the RecT, or derivative or variant thereof, comprises an amino acid sequences with at least 90% similarity to amino acid sequences selected from the group consisting of SEQ ID NO:9.

In certain embodiments, the fusion protein comprises a recombination protein comprising an amino acid sequence at least 75% similar, or at least 75% identical to a recombination protein of SEQ ID NO: 166 to SEQ ID NO:491, a recombination protein of Table 9, a recombination protein of SEQ ID NO: 179, SEQ ID NO: 185, SEQ ID NO:205, SEQ ID NO:321, SEQ ID NO:353, SEQ ID NO:359, SEQ ID NO: 366, SEQ ID NO:424, or SEQ ID NO:479, or a recombination protein of SEQ ID NO:166, SEQ ID NO:168, SEQ ID NO:169, SEQ ID NO:170, SEQ ID NO: 171, SEQ ID NO:241, SEQ ID NO:253, SEQ ID NO:290, SEQ ID NO:408, SEQ ID NO: 411, or SEQ ID NO:442. In certain embodiments the fusion protein comprises a recombination protein comprising a sequence having at least 80%, at least 85%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% similarity or identity to the above referenced recombination proteins.

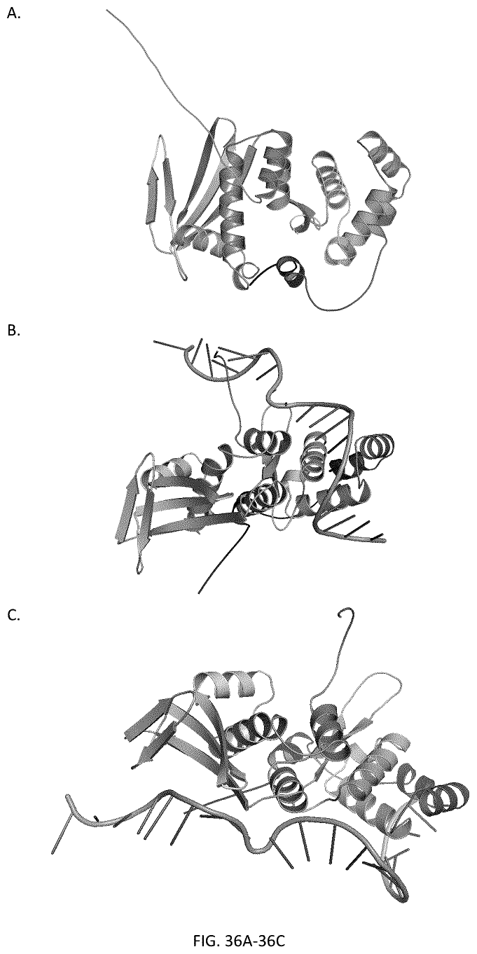

In certain embodiments, the fusion protein comprises a truncated recombination protein of SEQ ID NO:166 to SEQ ID NO:491. Truncations may be from either the C-terminal or N-terminal ends, or both. For example, as demonstrated in Example 6 below, a diverse set of truncations from either end or both provided a functional product. In some embodiments, one or more (2, 3, 4, 5, 6, 7, 8, 9, 10, 12, 14, 16, 18, 20, 25, 30, 40, 50, 60, 70, 80, 90, 100, 110, 120 or more) amino acids may be truncated from the C-terminal, N-terminal ends as compared to the wild-type sequence. The invention includes guidance as to suitability of truncations, substitutions, deletions, and insertions, for example with reference to FIGS. 36, 37, 44, and by comparison of recombination protein sequences herein.

The invention provides recombination proteins capable of improved gene editing activity. In certain embodiments, systems comprising a recombination protein of the invention are capable of editing efficiency equal to or greater than systems comprising EcRecT, for example, without limitation, 1.2×, 1.5×, 1.7×, 2×, 2.5×, 3×, or more compared to EcRecT. In certain embodiments, systems comprising a recombination protein of the invention provide cell viability equal to or greater than systems comprising EcRecT, for example, without limitation, 1.1×, 1.2×, 1.3×, 1.5×, 1.7×, 2×, 2.5×, 3×, or more compared to EcRecT.

In some embodiments, the recombination protein comprises a tyrosine recombinase or functional fragment thereof. In some embodiments, the recombination protein comprises a serine recombinase or functional fragment thereof. In some embodiments, the recombination protein comprises an integrase, resolvase, or invertase, or functional fragment thereof. In some embodiments, the recombinase protein comprises a site-specific recombinase protein or functional fragment thereof. In some embodiments, the recombination protein comprises an exonuclease or functional fragment thereof. In some embodiments, the recombination protein comprises an ssDNA-binding protein or functional fragment thereof. In certain embodiments, the fusion protein comprises without limitation, Hin, Gin, Tn3, β/six, CinH, Min, ParA, γδ, Bxb1, ϕC31, TP901-1, TGI, Wβ, ϕ370.1, ϕK38, ϕBT1, R4, ϕRV1, ϕFC1, MR11, A118, U153, Bxz2, gp29, Cre, Dre, Vika, Flp, Kw, SprA, HK022, P22, L1, or L5 or a homolog of any of such proteins or functional fragment thereof. Such recombinases, which may be classified in the art as integrases, resolvases, or invertases, may share substructures and activities with exonucleases and SSBs and be used according to the invention.

In the fusion protein, the microbial recombination protein may be linked to cither terminus of the aptamer binding protein in any orientation (e.g., N-terminus to C-terminus, C-terminus to N-terminus, N-terminus to N-terminus). In select embodiments, the microbial recombination protein N-terminus is linked to the aptamer binding protein C-terminus. Thus, the overall fusion protein from N- to C-terminus comprises the aptamer binding protein (N- to C-terminus) linked to the microbial recombination protein (N- to C-terminus).

In some embodiments, the fusion protein further comprises a linker between the microbial recombination protein and the aptamer binding protein. The linkers may comprise any amino acid sequence of any length. The linkers may be flexible such that they do not constrain either of the two components they link together in any particular orientation. The linkers may essentially act as a spacer. In select embodiments, the linker links the C-terminus of the microbial recombination protein to the N-terminus of the aptamer binding protein. In select embodiments, the linker comprises the amino acid sequence of the 16-residue XTEN linker, SGSETPGTSESATPES (SEQ ID NO:15) or the 37-residue EXTEN linker, SASGGSSGGSSGSETPGTSESATPESSGGSSGGSGGS (SEQ ID NO:148).

In some embodiments, the fusion protein further comprises a nuclear localization sequence (NLS). The nuclear localization sequence may be at any location within the fusion protein (e.g., C-terminal of the aptamer binding protein, N-terminal of the aptamer binding protein, C-terminal of the microbial recombination protein). In select embodiments, the nuclear localization sequence is linked to the C-terminus of the microbial recombination protein. A number of nuclear localization sequences are known in the art (see, e.g., Lange, A., et al., J Biol Chem. 2007; 282 (8): 5101-5105, incorporated herein by reference) and may be used in connection with the present disclosure. The nuclear localization sequence may be the SV40 NLS, PKKKRKV (SEQ ID NO:16); the Ty1 NLS, NSKKRSLEDNETEIKVSRDTWNTKNMRSLEPPRSKKRIH (SEQ ID NO:17); the c-Myc NLS, PAAKRVKLD (SEQ ID NO:18); the biSV40 NLS, KRTADGSEFESPKKKRKV (SEQ ID NO:19); and the Mut NLS, PEKKRRRPSGSVPVLARPSPPKAGKSSCI (SEQ ID NO:20). In select embodiments, the nuclear localization sequence is the SV40 NLS, PKKKRKV (SEQ ID NO:16).

The Cas protein and the fusion protein are desirably included in a single composition alone, in combination with each other, and/or the polynucleotide(s) (e.g., a vector) comprising the guide RNA sequence and the aptamer sequence. The Cas protein and/or the fusion protein may or may not be physically or chemically bound to the polynucleotide. The Cas protein and/or the microbial recombination protein can be associated with a polynucleotide using any suitable method for protein-protein linking or protein-virus linking known in the art.

The disclosure further provides compositions and vectors comprising a polynucleotide comprising a nucleic acid sequence encoding a fusion protein comprising a microbial recombination protein functionally linked to an RNA aptamer binding protein.

The compositions or vectors may further comprise at least one or both of a polynucleotide comprising a nucleic acid sequence encoding a Cas protein and a nucleic acid molecule comprising a guide RNA sequence that is complementary to a target DNA sequence. In some embodiments, the nucleic acid molecule comprising a guide RNA sequence further comprises at least one RNA aptamer sequence. In some embodiments, the polynucleotide comprising a nucleic acid sequence encoding a Cas protein further comprises a sequence encoding at least one peptide aptamer sequence.

Descriptions of the nucleic acid molecule comprising a guide RNA sequence, the aptamer sequences, the Cas proteins, the microbial recombination proteins, and the aptamer binding proteins set forth above in connection with the inventive system also are applicable to the polynucleotides of the recited compositions and vectors.

The nucleic acid sequence encoding the Cas protein and/or the nucleic acid sequence encoding a fusion protein comprising a microbial recombination protein functionally linked to an aptamer binding protein can be provided to a cell on the same vector (e.g., in cis) as the nucleic acid molecule comprising the guide RNA sequence and/or the RNA aptamer sequence. In such embodiments, a unidirectional promoter can be used to control expression of each nucleic acid sequence. In another embodiment, a combination of bidirectional and unidirectional promoters can be used to control expression of multiple nucleic acid sequences.

In other embodiments, a nucleic acid sequence encoding the Cas protein, the nucleic acid sequence encoding a fusion protein comprising a microbial recombination protein functionally linked to an aptamer binding protein, and the nucleic acid molecule comprising the guide RNA sequence and/or the RNA aptamer sequence can be provided to a cell on separate vectors (e.g., in trans). Each of the nucleic acid sequences in each of the separate vectors can comprise the same or different expression control sequences. The separate vectors can be provided to cells simultaneously or sequentially.

The vector(s) comprising the nucleic acid sequences encoding the Cas protein and encoding a fusion protein comprising a microbial recombination protein functionally linked to an aptamer binding protein can be introduced into a host cell that is capable of expressing the polypeptide encoded thereby, including any suitable prokaryotic or eukaryotic cell. As such, the disclosure provides an isolated cell comprising the vector or nucleic acid sequences disclosed herein. Preferred host cells are those that can be easily and reliably grown, have reasonably fast growth rates, have well characterized expression systems, and can be transformed or transfected easily and efficiently. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Endivia. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Exemplary insect cells include Sf-9 and HIS (Invitrogen, Carlsbad, Calif.) and are described in, for example, Kitts et al., Biotechniques, 14:810-817 (1993); Lucklow, Curr. Opin. Biotechnol., 4:564-572 (1993); and Lucklow et al., J. Virol., 67:4566-4579 (1993), incorporated herein by reference. Desirably, the host cell is a mammalian cell, and in some embodiments, the host cell is a human cell. A number of suitable mammalian and human host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97:4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate, rodent, and human cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, HEK, A549, HepG2, mouse L-929 cells, and BHK or HaK hamster cell lines. Methods for selecting suitable mammalian host cells and methods for transformation, culture, amplification, screening, and purification of cells are known in the art.

### 3. Methods of Altering Target DNA

The disclosure also provides a method of altering a target DNA. In some embodiments, the method alters genomic DNA sequence in a cell, although any desired nucleic acid may be modified. When applied to DNA contained in cells, the method comprises introducing the systems, compositions, or vectors described herein into a cell comprising a target genomic DNA sequence. Descriptions of the nucleic acid molecule comprising a guide RNA sequence, the Cas proteins, the microbial recombination proteins, the recruitment systems, and polynucleotides encoding thereof, the cell, the target genomic DNA sequence, and components thereof, set forth above in connection with the inventive system are also applicable to the method of altering a target genomic DNA sequence in a cell. The systems, composition or vectors may be introduced in any manner known in the art including, but not limited to, chemical transfection, electroporation, microinjection, biolistic delivery via gene guns, or magnetic-assisted transfection, depending on the cell type.

Upon introducing the systems described herein into a cell comprising a target genomic DNA sequence, the guide RNA sequence binds to the target genomic DNA sequence in the cell genome, the Cas protein associates with the guide RNA and may induce a double strand break or single strand nick in the target genomic DNA sequence and the aptamer recruits the microbial recombination proteins to the target genomic DNA sequence through the aptamer binding protein of the fusion protein, thereby altering the target genomic DNA sequence in the cell. When introducing the compositions, or vectors described herein into the cell, the nucleic acid molecule comprising a guide RNA sequence, the Cas9 protein, and the fusion protein are first expressed in the cell.

In some embodiments, the cell is in an organism or host, such that introducing the disclosed systems, compositions, vectors into the cell comprises administration to a subject. The method may comprise providing or administering to the subject, in vivo, or by transplantation of ex vivo treated cells, systems, compositions, vectors of the present system.

A “subject” may be human or non-human and may include, for example, plants or animal strains or species used as “model systems” for research purposes, such a mouse model as described herein. Likewise, subject may include either adults or juveniles (e.g., children). Moreover, subject may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice, and guinea pigs, and the like. Examples of non-mammals include, but are not limited to, birds, fish and the like. In one embodiment of the methods and compositions provided herein, the mammal is a human. Plants include without limitation sugar cane, corn, wheat, rice, oil palm fruit, potatoes, soybeans, vegetables, cassava, sugar beets, tomatoes, barley, bananas, watermelon, onions, sweet potatoes, cucumbers, apples, seed cotton, oranges, and the like.

As used herein, the terms “providing”, “administering,” “introducing,” are used interchangeably herein and refer to the placement of the systems of the disclosure into a subject by a method or route which results in at least partial localization of the system to a desired site. The systems can be administered by any appropriate route which results in delivery to a desired location in the subject.

The phrase “altering a DNA sequence,” as used herein, refers to modifying at least one physical feature of a DNA sequence of interest. DNA alterations include, for example, single or double strand DNA breaks, deletion, or insertion of one or more nucleotides, and other modifications that affect the structural integrity or nucleotide sequence of the DNA sequence. The modifications of a target sequence in genomic DNA may lead to, for example, gene correction, gene replacement, gene tagging, transgene insertion, nucleotide deletion, gene disruption, gene mutation, gene knock-down, and the like.

In some embodiments, the systems and methods described herein may be used to correct one or more defects or mutations in a gene (referred to as “gene correction”). In such cases, the target genomic DNA sequence encodes a defective version of a gene, and the system further comprises a donor nucleic acid molecule which encodes a wild-type or corrected version of the gene. Thus, in other words, the target genomic DNA sequence is a “disease-associated” gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. Examples of genes responsible for such “single gene” or “monogenic” diseases include, but are not limited to, adenosine deaminase, α-1 antitrypsin, cystic fibrosis transmembrane conductance regulator (CFTR), β-hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). Other single gene or monogenic diseases are known in the art and described in, e.g., Chial, H. Rare Genetic Disorders: Learning About Genetic Disease Through Gene Mapping, SNPs, and Microarray Data, Nature Education 1 (1): 192 (2008), incorporated herein by reference; Online Mendelian Inheritance in Man (OMIM); and the Human Gene Mutation Database (HGMD).

In another embodiment, the target genomic DNA sequence can comprise a gene, the mutation of which contributes to a particular disease in combination with mutations in other genes. Diseases caused by the contribution of multiple genes which lack simple (e.g., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects.

In another embodiment, the method of altering a target genomic DNA sequence can be used to delete nucleic acids from a target sequence in a cell by cleaving the target sequence and allowing the cell to repair the cleaved sequence in the absence of an exogenously provided donor nucleic acid molecule. Deletion of a nucleic acid sequence in this manner can be used in a variety of applications, such as, for example, to remove disease-causing trinucleotide repeat sequences in neurons, to create gene knock-outs or knock-downs, and to generate mutations for disease models in research.

The term “donor nucleic acid molecule” refers to a nucleotide sequence that is inserted into the target DNA (e.g., genomic DNA). As described above the donor DNA may include, for example, a gene or part of a gene, a sequence encoding a tag or localization sequence, or a regulating element. The donor nucleic acid molecule may be of any length. In some embodiments, the donor nucleic acid molecule is between 10 and 10,000 nucleotides in length. For example, between about 100 and 5,000 nucleotides in length, between about 200 and 2,000 nucleotides in length, between about 500 and 1,000 nucleotides in length, between about 500 and 5,000 nucleotides in length, between about 1,000 and 5,000 nucleotides in length, or between about 1,000 and 10,000 nucleotides in length,

The disclosed systems and methods overcome challenges encountered during conventional gene editing, including low efficiency and off-target events, particularly with kilobase-scale nucleic acids. In some embodiments, the disclosed systems and methods improve the efficiency of gene editing. For example, the disclosed systems and methods can have a 2- to 10-fold increase in efficiency over conventional CRISPR-Cas9 systems and methods, as shown in Examples 2, 3, and 5. In some embodiments, the improvement in efficiency is accompanied by a reduction in off-target events. The off-target events may be reduced by greater than 50% compared to conventional CRISPR-Cas9 systems and methods, for example, a reduction of off-target events by about 90% is shown in Example 3. Another aspect of increasing the overall accuracy of a gene editing system is reducing the on-target insertion-deletions (indels), a byproduct of HDR editing. In some embodiments, the disclosed systems and methods reduce the on-target indels by greater than 90% compared to conventional CRISPR-Cas9 systems and methods, as shown in Example 3.

The disclosure further provides kits containing one or more reagents or other components useful, necessary, or sufficient for practicing any of the methods described herein. For example, kits may include CRISPR reagents (Cas protein, guide RNA, vectors, compositions, etc.), recombineering reagents (recombination protein-aptamer binding protein fusion protein, the aptamer sequence, vectors, compositions, etc.) transfection or administration reagents, negative and positive control samples (e.g., cells, template DNA), cells, containers housing one or more components (e.g., microcentrifuge tubes, boxes), detectable labels, detection and analysis instruments, software, instructions, and the like.

The RNAs may be delivered using adeno associated virus (AAV), lentivirus, adenovirus or other viral vector types, or combinations thereof. The RNAs can be packaged into one or more viral vectors. In some embodiments, the viral vector is delivered to the tissue of interest by, for example, an intramuscular injection, while other times the viral delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector chose, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered salinc), a pharmaceutically-acceptable excipient, and/or other compounds known in the art. Such a dosage formulation is readily ascertainable by one skilled in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin, and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹² particles), more preferably at least about 1×10¹⁰ particles, more preferably at least about 1×10⁸ particles (e.g., about 1×10⁸-1×10¹¹ particles or about 1×10⁸-1×10¹² particles), and most preferably at least about 1×10⁰ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ articles). Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10¹⁰ functional AAV/ml solution. The dosage may be adjusted to balance the therapeutic benefit against any side effects. In an embodiment herein, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In an embodiment herein the delivery is via a plasmid. In such plasmid compositions, the dosage should be a sufficient amount of plasmid to elicit a response. For instance, suitable quantities of plasmid DNA in plasmid compositions can be from about 0.1 to about 2 mg, or from about 1 μg to about 10 μg.

The doses herein are based on an average 70 kg individual. The frequency of administration is within the ambit of the medical or veterinary practitioner (e.g., physician, veterinarian), or scientist skilled in the art. Mice used in experiments are about 20 g. From that which is administered to a 20 g mouse, one can extrapolate to a 70 kg individual.

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses may be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media was changed to OptiMEM (serum-free) media and transfection was done 4 hours later. Cells were transfected with 10 μg of lentiviral transfer plasmid (pCasES10) and the following packaging plasmids: 5 μg of pMD2. G (VSV-g pseudotype), and 7.5 ug of psPAX2 (gag/pol/rev/tat). Transfection was done in 4 mL OptiMEM with a cationic lipid delivery agent (50 uL Lipofectamine 2000 and 100 uL Plus reagent). After 6 hours, the media was changed to antibiotic-free DMEM with 10% fetal bovine serum.

Lentivirus may be purified as follows. Viral supernatants were harvested after 48 hours. Supernatants were first cleared of debris and filtered through a 0.45 um low protein binding (PVDF) filter. They were then spun in an ultracentrifuge for 2 hours at 24,000 rpm. Viral pellets were resuspended in 50 ul of DMEM overnight at 4 C. They were then aliquotted and immediately frozen at −80 C.

In another embodiment, minimal non-primate lentiviral vectors based on the equine infectious anemia virus (EIAV) are also contemplated, especially for ocular gene therapy (see, e.g., Balagaan, J Genc Med 2006; 8:275-285, Published online 21 Nov. 2005 in Wiley Interscience; available at the website: interscience.wiley.com. DOI: 10.1002/jgm.845). In another embodiment, RetinoStat®, an equine infectious anemia virus-based lentiviral gene therapy vector that expresses angiostatic proteins endostain and angiostatin that is delivered via a subretinal injection for the treatment of the web form of age-related macular degeneration is also contemplated (see, e.g., Binley et al., HUMAN GENE THERAPY 23:980-991 (September 2012)) may be modified for the system of the present invention.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, scc, e.g., US Patent Publication Nos. US20110293571; US20110293571, US20040013648, US20070025970, US20090111106 and U.S. Pat. No. 7,259,015.

Several types of particle delivery systems and/or formulations are known to be useful in a diverse spectrum of biomedical applications. In general, a particle is defined as a small object that behaves as a whole unit with respect to its transport and properties. Particles are further classified according to diameter Coarse particles cover a range between 2,500 and 10,000 nanometers. Fine particles are sized between 100 and 2,500 nanometers. Ultrafine particles, or nanoparticles, are generally between 1 and 100 nanometers in size. The basis of the 100-nm limit is the fact that novel properties that differentiate particles from the bulk material typically develop at a critical length scale of under 100 nm.

As used herein, a particle delivery system/formulation is defined as any biological delivery system/formulation which includes a particle in accordance with the present invention. A particle in accordance with the present invention is any entity having a greatest dimension (e.g., diameter) of less than 100 microns (μm). In some embodiments, inventive particles have a greatest dimension of less than 10 In some embodiments, inventive particles have a greatest dimension of less than 2000 nanometers (nm). In some embodiments, inventive particles have a greatest dimension of less than 1000 nanometers (nm). In some embodiments, inventive particles have a greatest dimension of less than 900 nm, 800 nm, 700 nm, 600 nm, 500 nm, 400 nm, 300 nm, 200 nm, or 100 nm. Typically, inventive particles have a greatest dimension (e.g., diameter) of 500 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less. In some embodiments, inventive particles have a greatest dimension (e.g., diameter) of 100 nm or less. Smaller particles, e.g., having a greatest dimension of 50 nm or less are used in some embodiments of the invention. In some embodiments, inventive particles have a greatest dimension ranging between 25 nm and 200 nm.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to one or more RNAs and/or vectors encoding the same, and may include additional components, carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS).

Particles delivery systems within the scope of the present invention may be provided in any form, including but not limited to solid, semi-solid, emulsion, or colloidal particles. As such any of the delivery systems described herein, including but not limited to, e.g., lipid-based systems, liposomes, micelles, microvesicles, exosomes, or gene gun may be provided as particle delivery systems within the scope of the present invention.

In terms of this invention, it is preferred to have one or more components of the system delivered using nanoparticles or lipid envelopes. CRISPR enzyme mRNA and guide RNA may be delivered simultaneously using nanoparticles or lipid envelopes. Other delivery systems or vectors may be used in conjunction with the nanoparticle aspects of the invention.

In general, a “nanoparticle” refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, nanoparticles of the invention have a greatest dimension ranging between 35 nm and 60 nm.

Nanoparticles encompassed in the present invention may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention.

Semi-solid and soft nanoparticles have been manufactured, and are within the scope of the present invention. A prototype nanoparticle of semi-solid nature is the liposome. Various types of liposome nanoparticles are currently used clinically as delivery systems for anticancer drugs and vaccines. Nanoparticles with one half hydrophilic and the other half hydrophobic are termed Janus particles and are particularly effective for stabilizing emulsions. They can self-assemble at water/oil interfaces and act as solid surfactants.

For example, Su X, Fricke J, Kavanagh D G, Irvine D J (“In vitro and in vivo mRNA delivery using lipid-enveloped pH-responsive polymer nanoparticles” Mol Pharm. 2011 Jun. 6; 8 (3): 774-87. doi:10.1021/mp100390w. Epub 2011 Apr. 1) describes biodegradable core-shell structured nanoparticles with a poly(β-amino ester) (PBAE) core enveloped by a phospholipid bilayer shell. These were developed for in vivo mRNA delivery. The pH-responsive PBAE component was chosen to promote endosome disruption, while the lipid surface layer was selected to minimize toxicity of the polycation core. Such are, therefore, preferred for delivering RNA of the present invention.

In one embodiment, nanoparticles based on self-assembling bioadhesive polymers are contemplated, which may be applied to oral delivery of peptides, intravenous delivery of peptides and nasal delivery of peptides, all to the brain. Other embodiments, such as oral absorption and ocular deliver of hydrophobic drugs are also contemplated. The molecular envelope technology involves an engineered polymer envelope which is protected and delivered to the site of the disease (see, e.g., Mazza, M. et al. ACSNano, 2013. 7 (2): 1016-1026; Siew, A., et al. Mol Pharm, 2012. 9 (1): 14-28; Lalatsa, A., et al. J Contr Rel, 2012. 161 (2): 523-36; Lalatsa, A., et al., Mol Pharm, 2012. 9 (6): 1665-80; Lalatsa, A., et al. Mol Pharm, 2012. 9 (6): 1764-74; Garrett, N. L., et al. J Biophotonics, 2012. 5 (5-6): 458-68; Garrett, N. L., et al. J Raman Spect, 2012. 43 (5): 681-688; Ahmad, S., et al. J Royal Soc Interface 2010. 7: S423-33; Uchegbu, I. F. Expert Opin Drug Deliv, 2006. 3 (5): 629-40; Qu, X., et al. Biomacromolecules, 2006. 7 (12): 3452-9 and Uchegbu, I. F., et al. Int J Pharm, 2001. 224:185-199). Doses of about 5 mg/kg are contemplated, with single or multiple doses, depending on the target tissue.

In one embodiment, nanoparticles that can deliver RNA to a cancer cell to stop tumor growth developed by Dan Anderson's lab at MIT may be used/and or adapted to the CRISPR Cas system of the present invention. In particular, the Anderson lab developed fully automated, combinatorial systems for the synthesis, purification, characterization, and formulation of new biomaterials and nanoformulations. Sec, e.g., Alabi et al., Proc Natl Acad Sci USA. 2013 Aug. 6; 110 (32): 12881-6; Zhang et al., Adv Mater. 2013 Sep. 6; 25 (33): 4641-5; Jiang et al., Nano Lett. 2013 Mar. 13; 13 (3): 1059-64; Karagiannis et al., ACS Nano. 2012 Oct. 23; 6 (10): 8484-7; Whitehead et al., ACS Nano. 2012 Aug. 28; 6 (8): 6922-9 and Lee et al., Nat Nanotechnol. 2012 Jun. 3; 7 (6): 389-93.

U.S. patent application No. 20110293703 relates to lipidoid compounds are also particularly useful in the administration of polynucleotides, which may be applied to deliver the CRISPR Cas system of the present invention. In one aspect, the aminoalcohol lipidoid compounds are combined with an agent to be delivered to a cell or a subject to form microparticles, nanoparticles, liposomes, or micelles. The agent to be delivered by the particles, liposomes, or micelles may be in the form of a gas, liquid, or solid, and the agent may be a polynucleotide, protein, peptide, or small molecule. The minoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

US Patent Publication No. 0110293703 also provides methods of preparing the aminoalcohol lipidoid compounds. One or more equivalents of an amine are allowed to react with one or more equivalents of an epoxide-terminated compound under suitable conditions to form an aminoalcohol lipidoid compound of the present invention. In certain embodiments, all the amino groups of the amine are fully reacted with the epoxide-terminated compound to form tertiary amines. In other embodiments, all the amino groups of the amine are not fully reacted with the epoxide-terminated compound to form tertiary amines thereby resulting in primary or secondary amines in the aminoalcohol lipidoid compound. These primary or secondary amines are left as is or may be reacted with another electrophile such as a different epoxide-terminated compound. As will be appreciated by one skilled in the art, reacting an amine with less than excess of epoxide-terminated compound will result in a plurality of different aminoalcohol lipidoid compounds with various numbers of tails. Certain amines may be fully functionalized with two epoxide-derived compound tails while other molecules will not be completely functionalized with epoxide-derived compound tails. For example, a diamine or polyamine may include one, two, three, or four epoxide-derived compound tails off the various amino moieties of the molecule resulting in primary, secondary, and tertiary amines. In certain embodiments, all the amino groups are not fully functionalized. In certain embodiments, two of the same types of epoxide-terminated compounds are used. In other embodiments, two or more different epoxide-terminated compounds are used. The synthesis of the aminoalcohol lipidoid compounds is performed with or without solvent, and the synthesis may be performed at higher temperatures ranging from 30-100 C., preferably at approximately 50-90 C. The prepared aminoalcohol lipidoid compounds may be optionally purified. For example, the mixture of aminoalcohol lipidoid compounds may be purified to yield an aminoalcohol lipidoid compound with a particular number of epoxide-derived compound tails. Or the mixture may be purified to yield a particular stereo- or regioisomer. The aminoalcohol lipidoid compounds may also be alkylated using an alkyl halide (e.g., methyl iodide) or another alkylating agent, and/or they may be acylated.

US Patent Publication No. 0110293703 also provides libraries of aminoalcohol lipidoid compounds prepared by the inventive methods. These aminoalcohol lipidoid compounds may be prepared and/or screened using high-throughput techniques involving liquid handlers, robots, microtiter plates, computers, etc. In certain embodiments, the aminoalcohol lipidoid compounds are screened for their ability to transfect polynucleotides or other agents (e.g., proteins, peptides, small molecules) into the cell.

US Patent Publication No. 20130302401 relates to a class of poly(beta-amino alcohols) (PBAAs) has been prepared using combinatorial polymerization. The inventive PBAAs may be used in biotechnology and biomedical applications as coatings (such as coatings of films or multilayer films for medical devices or implants), additives, materials, excipients, non-biofouling agents, micropatterning agents, and cellular encapsulation agents. When used as surface coatings, these PBAAs elicited different levels of inflammation, both in vitro and in vivo, depending on their chemical structures. The large chemical diversity of this class of materials allowed us to identify polymer coatings that inhibit macrophage activation in vitro. Furthermore, these coatings reduce the recruitment of inflammatory cells, and reduce fibrosis, following the subcutaneous implantation of carboxylated polystyrene microparticles. These polymers may be used to form polyelectrolyte complex capsules for cell encapsulation. The invention may also have many other biological applications such as antimicrobial coatings, DNA or siRNA delivery, and stem cell tissue engineering. The teachings of US Patent Publication No. 20130302401 may be applied to the system of the present invention.

In another embodiment, lipid nanoparticles (LNPs) are contemplated. In particular, an antitransthyretin small interfering RNA encapsulated in lipid nanoparticles (see, e.g., Coelho et al., N Engl J Med 2013; 369:819-29) may be applied to the system of the present invention. Doses of about 0.01 to about 1 mg per kg of body weight administered intravenously are contemplated. Medications to reduce the risk of infusion-related reactions are contemplated, such as dexamethasone, acetaminophen, diphenhydramine or cetirizine, and ranitidine are contemplated. Multiple doses of about 0.3 mg per kilogram every 4 weeks for five doses are also contemplated. Lipids include, but are not limited to, DLin-KC2-DMA4, C12-200 and colipids disteroylphosphatidyl choline, cholesterol, and PEG-DMG may be formulated RNA instead of siRNA (see, e.g., Novobrantseva, Molecular Therapy—Nucleic Acids (2012) 1, e4; doi:10.1038/mtna.2011.3) using a spontaneous vesicle formation procedure. The component molar ratio may be about 50/10/38.5/1.5 (DLin-KC2-DMA or C12-200/disteroylphosphatidyl choline/cholesterol/PEG-DMG). The final lipid: siRNA weight ratio may be ˜12:1 and 9:1 in the case of DLin-KC2-DMA and C12-200 lipid nanoparticles (LNPs), respectively. The formulations may have mean particle diameters of ^{˜}80 nm with >90% entrapment efficiency. A 3 mg/kg dose may be contemplated.

LNPs have been shown to be highly effective in delivering siRNAs to the liver (see, e.g., Tabernero et al., Cancer Discovery, April 2013, Vol. 3, No. 4, pages 363-470) and are therefore contemplated for delivering CRISPR Cas to the liver. A dosage of about four doses of 6 mg/kg of the LNP (or RNA of the CRISPR-Cas) every two weeks may be contemplated. Tabernero et al. demonstrated that tumor regression was observed after the first 2 cycles of LNPs dosed at 0.7 mg/kg, and by the end of 6 cycles the patient had achieved a partial response with complete regression of the lymph node metastasis and substantial shrinkage of the liver tumors. A complete response was obtained after 40 doses in this patient, who has remained in remission and completed treatment after receiving doses over 26 months. Two patients with RCC and extrahepatic sites of disease including kidney, lung, and lymph nodes that were progressing following prior therapy with VEGF pathway inhibitors had stable disease at all sites for approximately 8 to 12 months, and a patient with PNET and liver metastases continued on the extension study for 18 months (36 doses) with stable disease.

However, the charge of the LNP must be taken into consideration. As cationic lipids combined with negatively charged lipids to induce nonbilayer structures that facilitate intracellular delivery. Because charged LNPs are rapidly cleared from circulation following intravenous injection, ionizable cationic lipids with pKa values below 7 were developed (see, e.g., Rosin et al, Molecular Therapy, vol. 19, no. 12, pages 1286-220 December 2011). Negatively charged polymers such as siRNA oligonucleotides may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinolcyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminocthyl)-[1,3]-dioxolane (DLinKC2-DMA). It has been shown that LNP siRNA systems containing these lipids exhibit remarkably different gene silencing properties in hepatocytes in vivo, with potencies varying according to the series DLinKC2-DMA>DLinKDMA>DLinDMA>>DLinDAP employing a Factor VII gene silencing model (see, e.g., Rosin et al, Molecular Therapy, vol. 19, no. 12, pages 1286-220 December 2011). A dosage of 1 μg/ml levels may be contemplated, especially for a formulation containing DLinKC2-DMA. Preparation of LNPs and CRISPR Cas encapsulation may be used/and or adapted from Rosin et al, Molecular Therapy, vol. 19, no. 12, pages 1286-220 December 2011). The cationic lipids 1,2-dilincoyl-3-dimethylammonium-propane (DLinDAP), 1,2-dilinoleyloxy-3-N,N-dimethylaminopropane (DLinDMA), 1,2-dilinolcyloxykcto-N,N-dimethyl-3-aminopropanc (DLinK-DMA), 1,2-dilinoleyl-4-(2-dimethylaminocthyl)-[1,3]-dioxolane (DLinKC2-DMA), (3-0-[2″-(methoxypolyethyleneglycol 2000) succinoyl]-1,2-dimyristoyl-sn-glycol (PEG-S-DMG), and R-3-[(w-methoxy-poly(ethylene glycol) 2000) carbamoyl]-1,2-dimyristyloxlpropyl-3-amine (PEG-C-DOMG) may be provided by Tekmira Pharmaceuticals (Vancouver, Canada) or synthesized. Cholesterol may be purchased from Sigma (St Louis, Mo.). The specific CRISPR Cas RNA may be encapsulated in LNPs containing DLinDAP, DLinDMA, DLinK-DMA, and DLinKC2-DMA (cationic lipid: DSPC: CHOL: PEGS-DMG or PEG-C-DOMG at 40:10:40:10 molar ratios). When required, 0.2% SP-DiOC18 (Invitrogen, Burlington, Canada) may be incorporated to assess cellular uptake, intracellular delivery, and biodistribution. Encapsulation may be performed by dissolving lipid mixtures comprised of cationic lipid: DSPC: cholesterol: PEG-c-DOMG (40:10:40:10 molar ratio) in ethanol to a final lipid concentration of 10 mmol/1. This ethanol solution of lipid may be added dropwise to 50 mmol/1 citrate, pH 4.0 to form multilamellar vesicles to produce a final concentration of 30% ethanol vol/vol. Large unilamellar vesicles may be formed following extrusion of multilamellar vesicles through two stacked 80 nm Nuclepore polycarbonate filters using the Extruder (Northern Lipids, Vancouver, Canada). Encapsulation may be achieved by adding RNA dissolved at 2 mg/ml in 50 mmol/l citrate, pH 4.0 containing 30% ethanol vol/vol drop-wise to extruded preformed large unilamellar vesicles and incubation at 31° C. for 30 minutes with constant mixing to a final RNA/lipid weight ratio of 0.06/1 wt/wt. Removal of ethanol and neutralization of formulation buffer were performed by dialysis against phosphate-buffered saline (PBS), pH 7.4 for 16 hours using Spectra/Por 2 regenerated cellulose dialysis membranes. Nanoparticle size distribution may be determined by dynamic light scattering using a NICOMP 370 particle sizer, the vesicle/intensity modes, and Gaussian fitting (Nicomp Particle Sizing, Santa Barbara, Calif.). The particle size for all three LNP systems may be ^{˜}70 nm in diameter. siRNA encapsulation efficiency may be determined by removal of free siRNA using VivaPureD MiniH columns (Sartorius Stedim Biotech) from samples collected before and after dialysis. The encapsulated RNA may be extracted from the eluted nanoparticles and quantified at 260 nm. siRNA to lipid ratio was determined by measurement of cholesterol content in vesicles using the Cholesterol E enzymatic assay from Wako Chemicals USA (Richmond, Va.). PEGylated liposomes (or LNPs) can also be used for delivery.

Preparation of large LNPs may be used/and or adapted from Rosin et al, Molecular Therapy, vol. 19, no. 12, pages 1286-220 December 2011. A lipid premix solution (20.4 mg/ml total lipid concentration) may be prepared in ethanol containing DLinKC2-DMA, DSPC, and cholesterol at 50:10:38.5 molar ratios. Sodium acetate may be added to the lipid premix at a molar ratio of 0.75:1 (sodium acetate: DLinKC2-DMA). The lipids may be subsequently hydrated by combining the mixture with 1.85 volumes of citrate buffer (10 mmol/1, pH 3.0) with vigorous stirring, resulting in spontaneous liposome formation in aqueous buffer containing 35% ethanol. The liposome solution may be incubated at 37° C. to allow for time-dependent increase in particle size. Aliquots may be removed at various times during incubation to investigate changes in liposome size by dynamic light scattering (Zetasizer Nano ZS, Malvern Instruments, Worcestershire, UK). Once the desired particle size is achieved, an aqueous PEG lipid solution (stock=10 mg/ml PEG-DMG in 35% (vol/vol) ethanol) may be added to the liposome mixture to yield a final PEG molar concentration of 3.5% of total lipid. Upon addition of PEG-lipids, the liposomes should their size, effectively quenching further growth. RNA may then be added to the empty liposomes at an siRNA to total lipid ratio of approximately 1:10 (wt:wt), followed by incubation for 30 minutes at 37° C. to form loaded LNPs. The mixture may be subsequently dialyzed overnight in PBS and filtered with a 0.45-μm syringe filter.

Spherical Nucleic Acid (SNA™) constructs and other nanoparticles (particularly gold nanoparticles) are also contemplated as a means to delivery CRISPR/Cas system to intended targets. Significant data show that AuraSense Therapeutics' Spherical Nucleic Acid (SNA™) constructs, based upon nucleic acid-functionalized gold nanoparticles, are superior to alternative platforms based on multiple key success factors, such as:

High in vivo stability. Due to their dense loading, a majority of cargo (DNA or siRNA) remains bound to the constructs inside cells, conferring nucleic acid stability and resistance to enzymatic degradation.

Deliverability. For all cell types studied (e.g., neurons, tumor cell lines, etc.) the constructs demonstrate a transfection efficiency of 99% with no need for carriers or transfection agents.

Therapeutic targeting. The unique target binding affinity and specificity of the constructs allow exquisite specificity for matched target sequences (i.e., limited off-target effects).

Superior efficacy. The constructs significantly outperform leading conventional transfection reagents (Lipofectamine 2000 and Cytofectin).

Low toxicity. The constructs can enter a variety of cultured cells, primary cells, and tissues with no apparent toxicity.

No significant immune response. The constructs elicit minimal changes in global gene expression as measured by whole-genome microarray studies and cytokine-specific protein assays.

Chemical tailorability. Any number of single or combinatorial agents (e.g., proteins, peptides, small molecules) can be used to tailor the surface of the constructs.

This platform for nucleic acid-based therapeutics may be applicable to numerous disease states, including inflammation and infectious disease, cancer, skin disorders and cardiovascular disease.

Citable literature includes: Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495: S14-S16, Choi et al., Proc. Natl. Acad. Sci. USA. 2013 110 (19): 7625-7630, Jensen et al., Sci. Transl. Med. 5, 209ra152 (2013) and Mirkin, et al., Small, doi.org/10.1002/smll.201302143.

Self-assembling nanoparticles with siRNA may be constructed with polyethyleneimine (PEI) that is PEGylated with an Arg-Gly-Asp (RGD) peptide ligand attached at the distal end of the polyethylene glycol (PEG), for example, as a means to target tumor neovasculature expressing integrins and used to deliver siRNA inhibiting vascular endothelial growth factor receptor-2 (VEGF R2) expression and thereby tumor angiogenesis (see, e.g., Schiffelers et al., Nucleic Acids Research, 2004, Vol. 32, No. 19). Nanoplexes may be prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes. A dosage of about 100 to 200 mg of CRISPR Cas is envisioned for delivery in the self-assembling nanoparticles of Schiffelers et al.

The nanoplexes of Bartlett et al. (PNAS, Sep. 25, 2007, vol. 104, no. 39) may also be applied to the present invention. The nanoplexes of Bartlett et al. are prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes. The DOTA-siRNA of Bartlett et al. was synthesized as follows: 1,4,7,10-tetraazacyclododecane-1,4,7,10-tetraacetic acid mono(N-hydroxysuccinimide ester) (DOTA-NHSester) was ordered from Macrocyclics (Dallas, Tex.). The amine modified RNA sense strand with a 100-fold molar excess of DOTA-NHS-ester in carbonate buffer (pH 9) was added to a microcentrifuge tube. The contents were reacted by stirring for 4 h at room temperature. The DOTA-RNAsense conjugate was ethanol-precipitated, resuspended in water, and annealed to the unmodified antisense strand to yield DOTA-siRNA. All liquids were pretreated with Chelex-100 (Bio-Rad, Hercules, Calif.) to remove trace metal contaminants. Tf-targeted and nontargeted siRNA nanoparticles may be formed by using cyclodextrin-containing polycations. Typically, nanoparticles were formed in water at a charge ratio of 3 (+/−) and an siRNA concentration of 0.5 g/liter. One percent of the adamantane-PEG molecules on the surface of the targeted nanoparticles were modified with Tf (adamantane-PEG-Tf). The nanoparticles were suspended in a 5% (wt/vol) glucose carrier solution for injection.

Davis et al. (Nature, Vol 464, 15 Apr. 2010) conducts a siRNA clinical trial that uses a targeted nanoparticle-delivery system (clinical trial registration number NCT00689065). Patients with solid cancers refractory to standard-of-care therapies are administered doses of targeted nanoparticles on days 1, 3, 8 and 10 of a 21-day cycle by a 30-min intravenous infusion. The nanoparticles consist of a synthetic delivery system containing: (1) a linear, cyclodextrin-based polymer (CDP), (2) a human transferrin protein (TF) targeting ligand displayed on the exterior of the nanoparticle to engage TF receptors (TFR) on the surface of the cancer cells, (3) a hydrophilic polymer (polyethylene glycol (PEG) used to promote nanoparticle stability in biological fluids), and (4) siRNA designed to reduce the expression of the RRM2 (sequence used in the clinic was previously denoted siR2B+5). The TFR has long been known to be upregulated in malignant cells, and RRM2 is an established anti-cancer target. These nanoparticles (clinical version denoted as CALAA-01) have been shown to be well tolerated in multi-dosing studies in non-human primates. Although a single patient with chronic myeloid leukemia has been administered siRNA by liposomal delivery, Davis et al.'s clinical trial is the initial human trial to systemically deliver siRNA with a targeted delivery system and to treat patients with solid cancer. To ascertain whether the targeted delivery system can provide effective delivery of functional siRNA to human tumors, Davis et al. investigated biopsies from three patients from three different dosing cohorts; patients A, B and C, all of whom had metastatic melanoma and received CALAA-01 doses of 18, 24 and 30 mg m⁻² siRNA, respectively. Similar doses may also be contemplated for the CRISPR Cas system of the present invention. The delivery of the invention may be achieved with nanoparticles containing a linear, cyclodextrin-based polymer (CDP), a human transferrin protein (TF) targeting ligand displayed on the exterior of the nanoparticle to engage TF receptors (TFR) on the surface of the cancer cells and/or a hydrophilic polymer (for example, polyethylene glycol (PEG) used to promote nanoparticle stability in biological fluids).

Delivery or administration according to the invention can be performed with liposomes. Liposomes are spherical vesicle structures composed of a uni- or multilamellar lipid bilayer surrounding internal aqueous compartments and a relatively impermeable outer lipophilic phospholipid bilayer. Liposomes have gained considerable attention as drug delivery carriers because they are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Liposomes can be made from several different types of lipids; however, phospholipids are most commonly used to generate liposomes as drug carriers. Although liposome formation is spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review).

Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. Further, liposomes are prepared from hydrogenated egg phosphatidylcholine or egg phosphatidylcholine, cholesterol, and dicetyl phosphate, and their mean vesicle sizes were adjusted to about 50 and 100 nm. (see, e.g., Spuch and Navarro, Journal of Drug Delivery, vol. 2011, Article ID 469679, 12 pages, 2011. doi:10.1155/2011/469679 for review).

Conventional liposome formulation is mainly comprised of natural phospholipids and lipids such as 1,2-distearoryl-sn-glycero-3-phosphatidyl choline (DSPC), sphingomyelin, egg phosphatidylcholines and monosialoganglioside. Since this formulation is made up of phospholipids only, liposomal formulations have encountered many challenges, one of the ones being the instability in plasma. Several attempts to overcome these challenges have been made, specifically in the manipulation of the lipid membrane. One of these attempts focused on the manipulation of cholesterol. Addition of cholesterol to conventional formulations reduces rapid release of the encapsulated bioactive compound into the plasma or 1,2-diolcoyl-sn-glycero-3-phosphoethanolamine (DOPE) increases the stability (see, e.g., Spuch and Navarro, Journal of Drug Delivery, vol. 2011, Article ID 469679, 12 pages, 2011. doi:10.1155/2011/469679 for review).

In a particularly advantageous embodiment, Trojan Horse liposomes (also known as Molecular Trojan Horses) are desirable and protocols may be found at cshprotocols.cshlp.org/content/2010/4/pdb.prot5407.long. These particles allow delivery of a transgene to the entire brain after an intravascular injection. Without being bound by limitation, it is believed that neutral lipid particles with specific antibodies conjugated to surface allow crossing of the blood brain barrier via endocytosis. Applicant postulates utilizing Trojan Horse Liposomes to deliver the CRISPR family of nucleases to the brain via an intravascular injection, which would allow whole brain transgenic animals without the need for embryonic manipulation. About 1-5 g of nucleic acid molecule, e.g., DNA, RNA, may be contemplated for in vivo administration in liposomes.

In another embodiment, the system may be administered in liposomes, such as a stable nucleic-acid-lipid particle (SNALP) (see, e.g., Morrissey et al., Nature Biotechnology, Vol. 23, No. 8, August 2005). Daily intravenous injections of about 1, 3 or 5 mg/kg/day of a specific CRISPR Cas targeted in a SNALP are contemplated. The daily treatment may be over about three days and then weekly for about five weeks. In another embodiment, a specific CRISPR Cas encapsulated SNALP) administered by intravenous injection to at doses of abpit 1 or 2.5 mg/kg are also contemplated (see, e.g., Zimmerman et al., Nature Letters, Vol. 441, 4 May 2006). The SNALP formulation may contain the lipids 3-N-[(wmethoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio (see, e.g., Zimmerman et al., Nature Letters, Vol. 441, 4 May 2006).

In another embodiment, stable nucleic-acid-lipid particles (SNALPs) have proven to be effective delivery molecules to highly vascularized HepG2-derived liver tumors but not in poorly vascularized HCT-116 derived liver tumors (see, e.g., Li, Gene Therapy (2012) 19, 775-780). The SNALP liposomes may be prepared by formulating D-Lin-DMA and PEG-C-DMA with distcaroylphosphatidylcholine (DSPC), Cholesterol and siRNA using a 25:1 lipid/siRNA ratio and a 48/40/10/2 molar ratio of Cholesterol/D-Lin-DMA/DSPC/PEG-C-DMA. The resulted SNALP liposomes are about 80-100 nm in size.

In yet another embodiment, a SNALP may comprise synthetic cholesterol (Sigma-Aldrich, St Louis, Mo., USA), dipalmitoylphosphatidylcholine (Avanti Polar Lipids, Alabaster,

Ala., USA), 3-N-[(w-methoxy poly(ethylene glycol) 2000) carbamoyl]-1,2-dimyrestyloxypropylamine, and cationic 1,2-dilinoleyloxy-3-N,Ndimethylaminopropane (scc, e.g., Geisbert et al., Lancet 2010; 375:1896-905). A dosage of about 2 mg/kg total CRISPR Cas per dose administered as, for example, a bolus intravenous infusion may be contemplated.

In yet another embodiment, a SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinolcyloxy-3-(N;N-dimethyl)aminopropane (DLinDMA) (scc, e.g., Judge, J. Clin. Invest. 119:661-673 (2009)). Formulations used for in vivo studies may comprise a final lipid/RNA mass ratio of about 9:1.

Other cationic lipids, such as amino lipid 2,2-dilinoleyl-4-dimethylaminoethyl-[1,3]-dioxolane (DLin-KC2-DMA) may be utilized to encapsulate CRISPR Cas similar to SiRNA (scc, e.g., Jayaraman, Angew. Chem. Int. Ed. 2012, 51, 8529-8533). A preformed vesicle with the following lipid composition may be contemplated: amino lipid, di stearoylphosphatidylcholine (DSPC), cholesterol and (R)-2,3-bis(octadecyloxy) propyl-1-(methoxy poly(ethylene glycol) 2000) propylcarbamate (PEG-lipid) in the molar ratio 40/10/40/10, respectively, and a FVII siRNA/total lipid ratio of approximately 0.05 (w/w). To ensure a narrow particle size distribution in the range of 70-90 nm and a low polydispersity index of 0.11_0.04 (n=56), the particles may be extruded up to three times through 80 nm membranes prior to adding the CRISPR Cas RNA. Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

Any element of any suitable CRISPR/Cas gene editing system known in the art can be employed in the systems and methods described herein, as appropriate. CRISPR/Cas gene editing technology is described in detail in, for example, U.S. Patent Application Publication 2014/0068797; U.S. Pat. Nos. 8,697,359; 8,771,945; and 8,945,839; US2010/0076057; US2011/0189776; US2011/0223638; US2013/0130248; WO/2008/108989; WO/2010/054108; WO/2012/164565; WO/2013/098244; WO/2013/176772; US20150050699; US20150045546; US20150031134; US20150024500; US20140377868; US20140357530; US20140349400; US20140335620; US20140335063; US20140315985; US20140310830; US20140310828; US20140309487; US20140304853; US20140298547; US20140295556; US20140294773; US20140287938; US20140273234; US20140273232; US20140273231; US20140273230; US20140271987; US20140256046; US20140248702; US20140242702; US20140242700; US20140242699; US20140242664; US20140234972; US20140227787; US20140212869; US20140201857; US20140199767; US20140189896; US20140186958; US20140186919; US20140186843; US20140179770; US20140179006; and US20140170753, incorporated herein by reference.

Although the present invention and its advantages have been described in detail, it should be understood that various changes, substitutions, and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1-Materials and Methods

RecE/T Homolog Screening RefSeq non-redundant protein database was downloaded from NCBI on Oct. 29, 2019. The database was searched with E. coli Rac prophage RecT (NP_415865.1) and RecE (NP_415866.1) as queries using position-specific iterated (PSI)-BLAST¹ to retrieve protein homologs. Hits were clustered with CD-HIT2 and representative sequences were selected from each cluster for multiple alignment with MUSCLE³. Then, FastTree4 was used for maximum likelihood tree reconstruction with default parameters. A diverse set of RecET homologs were selected, synthesized by GenScript, and cloned into pMPH_MCP vectors for testing.

Plasmids construction pX330, pMPH and pU6-(BbsI)_CBh-Cas9-T2A-BFP plasmids were obtained from Addgene. Tested effector DNA fragments were ordered from IDT, Genewiz, and GenScript. The fragments were Gibson assembled into the backbones using NEBuilder HiFi DNA Assembly Master Mix (New England BioLabs). All sgRNAs (Table 1) were inserted into backbones using Golden Gate cloning. All constructs were sequence-verified with Sanger sequencing of prepped plasmids.

TABLE 1
Sequence for sgRNAs

| | Genomic | |
|---|---|---|
| Primer Name | Target | Sequence |
| sp-EMX1 | EMX1 | GTCACCTCCAATGACTAGGG (SEQ ID NO: 21) |
| sp-VEGFA | VEGFA | GGTGAGTGAGTGTGTGCGTG (SEQ ID NO: 22) |
| sp-DYNLT1 | DYNLT1 | AAGGCCATAGGCTGGACTGC (SEQ ID NO: 23) |
| sp-HSP90AA1 | HSP90AA1 | GTAGACTAATCTCTGGCTGA (SEQ ID NO: 24) |
| sp-OCT4 | OCT4 | TCTCCCATGCATTCAAACTG (SEQ ID NO: 25) |
| sp-AAVS1 | AAVS1 | ACCCCACAGTGGGGCCACTA (SEQ ID NO: 26) |
| nsp-EMX1-guide1 | EMX1 | GTCACCTCCAATGACTAGGG (SEQ ID NO: 27) |
| nsp-EMX1-guide2 | EMX1 | GTCACCTCCAATGACTAGGG (SEQ ID NO: 28) |
| nsp-DYNLT1-guide1 | DYNLT1 | AAGGCCATAGGCTGGACTGC (SEQ ID NO: 29) |
| nsp-DYNLT1-guide2 | DYNLT1 | GGCACTGACGATGCAGTACA (SEQ ID NO: 30) |
| nsp-HSP90AA1- | HSP90AA1 | GTAGACTAATCTCTGGCTGA (SEQ ID NO: 31) |
| guide1 | | |
| nsp-HSP90AA1- | HSP90AA1 | TCGTCATCTCCTTCAAGGGG (SEQ ID NO: 32) |
| guide2 | | |
| nsp-OCT4-guide1 | OCT4 | ATGCATGGGAGAGCCCAGAG (SEQ ID NO: 33) |
| nsp-OCT4-guide2 | OCT4 | GCCTGCCCTTCTAGGAATGG (SEQ ID NO: 34) |

Cell culture Human Embryonic Kidney (HEK) 293T, HeLa and HepG2 were maintained in Dulbecco's Modified Eagle's Medium (DMEM, Life Technologies), with 10% fetal bovine serum (FBS, HyClone), 100 U/mL penicillin, and 100 μg/mL streptomycin (Life Technologies) at 37° C. with 5% CO₂.

hES-H9 cells were maintained in mTeSR1 medium (StemCell Technologies) at 37° C. with 5% CO₂. Culture plates were pre-coated with Matrigel (Corning) 12 hours prior to use, and cells were supplemented with 10 μM Y27632 (Sigma) for the first 24 hours after passaging. Culture media was changed every 24 hours.

Transfection HEK293T cells were seeded into 96-well plates (Corning) 12-24 hours prior to transfection at a density of 30,000 cells/well, and 250 ng of total DNA was transfected per well. HeLa and HepG2 cells were seeded into 48-well plates (Corning) one day prior to transfection at a density of 50,000 and 30,000 cells/well respectively, and 400 ng of total DNA was transfected per well. Transfections were performed with Lipofectamine 3000 (Life Technologies) following the manufacturer's instructions.

Electroporation For hES-H9 related transfection experiments, P3 Primary Cell 4D-Nucleofector™ X Kit S (Lonza) was used following the manufacturer's protocol. For each reaction, 300,000 cells were nucleofected with 4 μg total DNA using the DC100 Nucleofector Program.

Fluorescence-activated cell sorting (FACS) mKate knock-in efficiency was analyzed on a CytoFLEX flow cytometer (Beckman Coulter; Stanford Stem Cell FACS Core). 72 hours after transfection, cells were washed once with PBS and dissociated with TrypLE Express Enzyme (Thermo Fisher Scientific). Cell suspension was then transferred to a 96-well U-bottom plate (Thermo Fisher Scientific) and centrifuged at 300×G for 5 minutes. After removing the supernatant, pelleted cells were resuspended with 50 μl 4% FBS in PBS, and cells were sorted within 30 minutes of preparation.

RFLP HEK293T cells were transfected with plasmid DNA and PCR templates and harvested after 72 hours for genomic DNA using the QuickExtract DNA Extraction Solution (Biosearch Technologies) following the manufacturer's protocol. The target genomic region was amplified using specific primers outside of the homology arms of the PCR template. PCR products were purified with Monarch PCR & DNA Cleanup Kit (New England BioLabs). 300 ng of purified product was digested with BsrGI (EMX1, New England BioLabs) or XbaI (VEGFA, NEB), and the digested products were analyzed on a 5% Mini-PROTEAN TBE gel (Bio-Rad).

Next-Generation Sequencing Library Preparation 72 hours after transfection, genomic DNA was extracted using QuickExtract DNA Extraction Solution (Biosearch Technologies). 200 ng total DNA was used for NGS library preparation. Genes of interest were amplified using specific primers (Table 2) for the first round PCR reaction. Illumina adapters and index barcodes were added to the fragments with a second round PCR using the primers listed in Table 2. Round 2 PCR products were purified by gel electrophoresis on a 2% agarose gel using the Monarch DNA Gel Extraction Kit (NEB). The purified product was quantified with Qubit dsDNA HS Assay Kit (Thermo Fisher) and sequenced on an Illumina MiSeq according to the manufacturer's instructions.

TABLE 2
Sequence for primers used for PCR template, RFLP and NGS

| | | Genomic | |
|---|---|---|---|
| Primer Name | Usage | Target | Sequence |
| EMX1-PCR-F | PCR | EMX1 | CATTCTGCCTCTCTGTATGGAAAAGAGC |
| | template | | (SEQ ID NO: 35) |
| EMX1-PCR-R | PCR | EMX1 | CCCATTGAACTACCTGGGCCTGATTC (SEQ |
| | template | | ID NO: 36) |
| VEGFA-PCR-F | PCR | VEGFA | AGGTTTGAATCATCACGCAGGC (SEQ ID |
| | template | | NO: 37) |
| VEGFA-PCR-R | PCR | VEGFA | ATTCAAGTGGGGAATGGCAAGC (SEQ ID |
| | template | | NO: 38) |
| DYNLT1- | PCR | DYNLT1 | TGCCGTAAATGCTGCTCTCT (SEQ ID NO: 39) |
| PCR-100 bp-F | template | | |
| DYNLT1- | PCR | DYNLT1 | AGACTTGCCAAGGTTCTTTGTG (SEQ ID |
| PCR-200 bp-F | template | | NO: 40) |
| DYNLT1- | PCR | DYNLT1 | AGTGACCTGTGTAATTATGCAGAAG (SEQ |
| PCR-400 bp-F | template | | ID NO: 41) |
| DYNLT1- | PCR | DYNLT1 | TGAAAGTGCCACAAAACAAAGAGA (SEQ |
| PCR-100 bp-R | template | | ID NO: 42) |
| DYNLT1- | PCR | DYNLT1 | AAGACAAGTGGCAACGCAG (SEQ ID |
| PCR-200 bp-R | template | | NO: 43) |
| DYNLT1- | PCR | DYNLT1 | CGTTTATGATACTATGCAGACTATGAAGAA |
| PCR-400 bp-R | template | | C (SEQ ID NO: 44) |
| HSP90AA1- | PCR | HSP90AA1 | ATGAAGATGACCCTACTGCTGAT (SEQ ID |
| PCR-100 bp-F | template | | NO: 45) |
| HSP90AA1- | PCR | HSP90AA1 | TACTGTCTTGAAAGCAGATAGAAACC (SEQ |
| PCR-200 bp-F | template | | ID NO: 46) |
| HSP90AA1- | PCR | HSP90AA1 | GCAGCAAAGAAACACCTGGA (SEQ ID |
| PCR-600 bp-R | template | | NO: 47) |
| HSP90AA1- | PCR | HSP90AA1 | GTTGTCATGCCATACAGACTTTTT (SEQ ID |
| PCR-100 bp-R | template | | NO: 48) |
| HSP90AA1- | PCR | HSP90AA1 | AGCATTACTAGCTCTGCTTTAGTG (SEQ ID |
| PCR-200 bp-R | template | | NO: 49) |
| HSP90AA1- | PCR | HSP90AA1 | TCCACAAGACTGGGTCTGAG (SEQ ID |
| PCR-600 bp-R | template | | NO: 50) |
| OCT4-PCR-F | PCR | OCT4 | GCGACTATGCACAACGAGAGG (SEQ ID |
| | template | | NO: 51) |
| OCT4-PCR-R | PCR | OCT4 | AAGTGTGTCTATCTACTGTGTCCCAG (SEQ |
| | template | | ID NO: 52) |
| AAVS1-PCR-F | PCR | AAVS1 | GATGCTCTTTCCGGAGCACT (SEQ ID |
| | template | | NO: 53) |
| AAVS1-PCR-R | PCR | AAVS1 | GCCAAGGACTCAAACCCAGAA (SEQ ID |
| | template | | NO: 54) |
| EMX1-RFLP-F | RFLP | EMX1 | TGGTGGATTTCGGACTACCCT (SEQ ID |
| | | | NO: 55) |
| EMX1-RFLP-R | RFLP | EMX1 | TTCGGACTGGAACCGTCAGC (SEQ ID |
| | | | NO: 56) |
| VEGFA- | RFLP | VEGFA | AGACGTTCCTTAGTGCTGGC (SEQ ID |
| RFLP-F | | | NO: 57) |
| VEGFA- | RFLP | VEGFA | AAAAGTTTCAGTGCGACGCC (SEQ ID |
| RFLP-R | | | NO: 58) |
| DYNLT1 KI | Junction | DYNLT1 | AGGAGGTCCCATCAGATGCT (SEQ ID |
| PCR-F | PCR | | NO: 59) |
| HSP90AA1 | Junction | HSP90AA1 | GGCTGGACAGCAAACATGGA (SEQ ID |
| KI PCR-F | PCR | | NO: 60) |
| AAVS1 KI | Junction | AAVS1 | GATGCTCTTTCCGGAGCACT (SEQ ID |
| PCR-F | PCR | | NO: 61) |
| Junction PCR | Junction | mKate | TTGCTGCCGTACATGAAGCTG (SEQ ID |
| universal-R | PCR | | NO: 62) |
| EMX1-NGS-F | NGS | EMX1 | CCATCTCATCCCTGCGTGTCTCCAGAAGA |
| | | | AGGGCTCCCATCAC (SEQ ID NO: 63) |
| EMX1-NGS-R | NGS | EMX1 | CCTCTCTATGGGCAGTCGGTGATgAGCAG |
| | | | CAAGCAGCACTCTG (SEQ ID NO: 64) |
| VEGFA-NGS- | NGS | VEGFA | CCATCTCATCCCTGCGTGTCTCCCAGCGT |
| F | | | CTTCGAGAGTGAGG (SEQ ID NO: 65) |
| VEGFA-NGS- | NGS | VEGFA | CCTCTCTATGGGCAGTCGGTGATgTTGGA |
| R | | | ATCCTGGAGTGACCC (SEQ ID NO: 66) |
| EMX-OT1-F | Off | EMX1 OT- | CCATCTCATCCCTGCGTGTCTCCACAAAA |
| | Target | 1 | GCTCCACATGCTAGGA (SEQ ID NO: 67) |
| EMX-OT1-R | Off | EMX1 OT- | CCTCTCTATGGGCAGTCGGTGATgGCTGA |
| | Target | 1 | CTTTGGGCTCCTTCT (SEQ ID NO: 68) |
| EMX-OT2-F | Off | EMX1 OT- | CCATCTCATCCCTGCGTGTCTCCACACAC |
| | Target | 2 | TCCCCAGGATCTCA (SEQ ID NO: 69) |
| EMX-OT2-R | Off | EMX1 OT- | CCTCTCTATGGGCAGTCGGTGATgAATGT |
| | Target | 2 | CAGCTGAAGCAGGCT (SEQ ID NO: 70) |
| EMX-OT3-F | Off | EMX1 OT- | CCATCTCATCCCTGCGTGTCTCCGGCTAC |
| | Target | 3 | CCTGACAACTGCTT (SEQ ID NO: 71) |
| EMX-OT3-R | Off | EMX1 OT- | CCTCTCTATGGGCAGTCGGTGATgAGGAC |
| | Target | 3 | AGACATGACAAGGCA (SEQ ID NO: 72) |
| VEGFA-OT1- | Off | VEGFA OT- | CCATCTCATCCCTGCGTGTCTCCGCAGGC |
| F | Target | 1 | AAGCTGTCAAGGGT (SEQ ID NO: 73) |
| VEGFA-OT1- | Off | VEGFA OT- | CCTCTCTATGGGCAGTCGGTGATgCCCTC |
| R | Target | 1 | ACACCCACACCCTCA (SEQ ID NO: 74) |
| VEGFA-OT2- | Off | VEGFA OT- | CCATCTCATCCCTGCGTGTCTCCGGAGG |
| F | Target | 2 | GGTGTCATCGTTCTG (SEQ ID NO: 75) |
| VEGFA-OT2- | Off | VEGFA OT- | CCTCTCTATGGGCAGTCGGTGATgCAAAT |
| R | Target | 2 | TGCGCCATAGCTGGG (SEQ ID NO: 76) |
| VEGFA-OT3- | Off | VEGFA OT- | CCATCTCATCCCTGCGTGTCTCCTGAGCG |
| F | Target | 3 | CTCTTCGTCTTTCC (SEQ ID NO: 77) |
| VEGFA-OT3- | Off | VEGFA OT- | CCTCTCTATGGGCAGTCGGTGATgGCCAG |
| R | Target | 3 | GAACACAGGAATGCTA (SEQ ID NO: 78) |
| | Junction | | TCCACCCCACAGTGGGGCAAGCTTCTGACC |
| | PCR | | TCTTCTCTTCCTCCCACAGGGCCT (SEQ ID |
| | | | NO: 163) |
| | PCR | | TTGACCTGCAGTCCAGCCTANGG (SEQ ID |
| | | | NO: 164) |
| | PCR | | CCACCGCAAATGCTTCTAGGNGG (SEQ ID |
| | | | NO: 165 |

High-throughput Sequencing Data Analysis Processed (demultiplexed, trimmed, and merged) sequencing reads were analyzed to determine editing outcomes using CRISPPResso2⁵ by aligning sequenced amplicons to reference and expected HDR amplicons. The quantification window was increased to 10 bp surrounding the expected cut site to better capture diverse editing outcomes, but substitutions were ignored to avoid inclusion of sequencing errors. Only reads containing no mismatches to the expected amplicon were considered for HDR quantification; reads containing indels that partially matched the expected amplicons were included in the overall reported indel frequency.

Statistical Analysis Unless otherwise stated, all statistical analysis and comparison were performed using t-test, with 1% false-discovery-rate (FDR) using two-stage step-up method of Benjamini, Krieger and Yekutieli (Benjamini, Y., et. al, Biometrika 93, 491-507 (2006), incorporated herein by reference). All experiments were performed in triplicates unless otherwise noted to ensure sufficient statistical power in the analysis.

Determination of editing at predicted Cas9 off-target sites To evaluate RecT/RecE off-target editing activity at known Cas9 off-target sites, same genomic DNA extracts for knock-in analysis were used as template for PCR amplification of top predicted off-targets sites (high scored as predicted CRISPOR, a web-based analysis tool) for the EMX1, VEGFA guides, primer sequences are listed in Table 2.

iGUIDE Off-target Analysis Genome-wide, unbiased off-target analysis was performed following the iGUIDE pipeline (Nobles, C. L., et al. Genome Biol 20, 14 (2019), incorporated herein by reference) based on Guide-seq invented previously (Tsai, S., et al. Nat Biotechnol 33, 187-197 (2015), incorporated herein by reference). HEK293T cells were transfected in 20 uL Lonza SF Cell Line Nucleofector Solution on a Lonza Nucleofector 4-D with program DS-150 according to the manufacturer's instructions. 300 ng of gRNA-Cas9 plasmids (or 150 ng of each gRNACas9n plasmid for the double nickase), 150 ng of the effector plasmids, and 5 pmol of double stranded oligonucleotides (dsODN) were transfected. Cells were harvested after 72 hrs for genomic DNA using Agencourt DNAdvance reagent kit. 400 ng of purified gDNA which was then fragmented to an average of 500 bp and ligated with adaptors using NEBNext Ultra II FS DNA Library Prep kit following manufacturer's instructions. Two rounds of nested anchored PCR from the oligo tag to the ligated adaptor sequence were performed to amplify targeted DNA, and the amplified library was purified, size-selected, and sequenced using Illumina Miseq V2 PE300. Sequencing data was analyzed using the published iGUIDE pipeline, with the addition of a downsampling step which ensures an unbiased comparison across samples.

### Example 2

In contrast to mammals, convenient recombineering-edit tools are available for bacteria, e.g., the phage lambda Red and RecE/T. Microbial recombineering has two major steps: template DNA is chewed back by exonucleases (Exo), then the single-strand annealing protein (SSAP) supports homology directed repair by the template, optionally facilitated by nuclease inhibitor. A system for RNA-guided targeting of RecE/T recombineering activities was developed and achieved kilobase (kb) human gene-editing without DNA cutting.

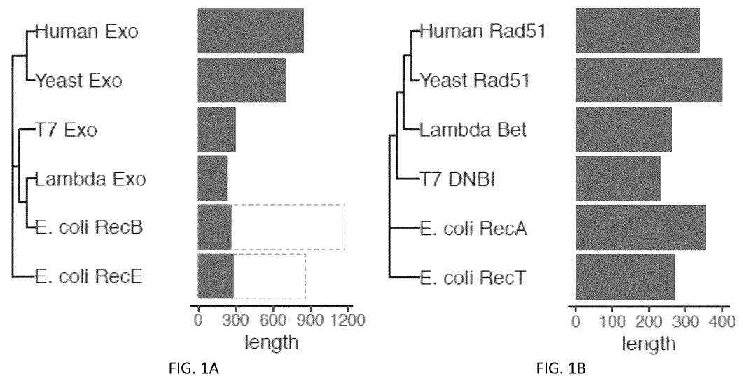

Candidate microbial systems with recombineering activities were surveyed. Two lines of reasoning guided the search: 1) Orthogonality: prioritizing proteins with minimal resemblance to mammalian repair enzymes; 2) Parsimony: focusing on systems with fewest interdependent components. Three protein families were identified: lambda Red, RecE/T, and phage T7 gp6 (Exo) and gp2.5 (SSAP) recombination machinery. Based on phylogenetic reconstruction, RecE/T proteins were determined to be the most distant from eukaryotic recombination proteins and among the most compact (FIG. 1). Thus, RecE/T systems were utilized for downstream analysis.

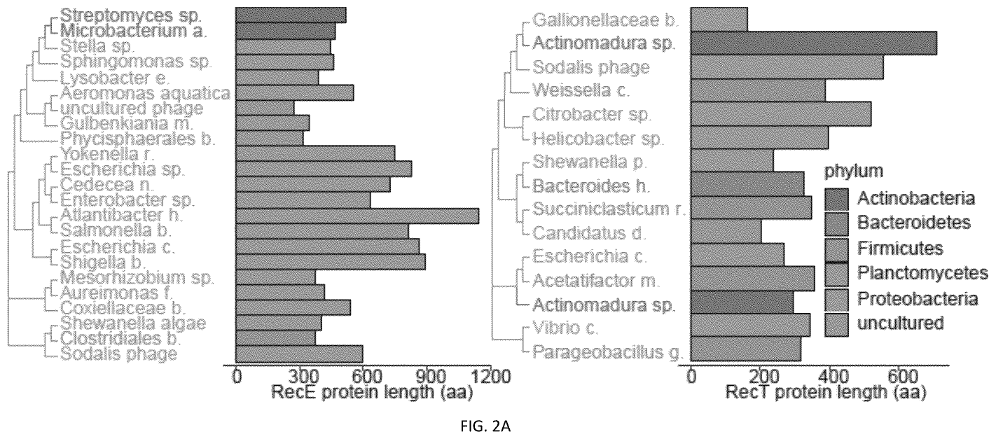

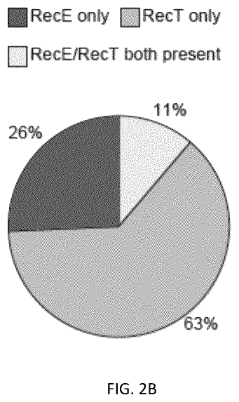

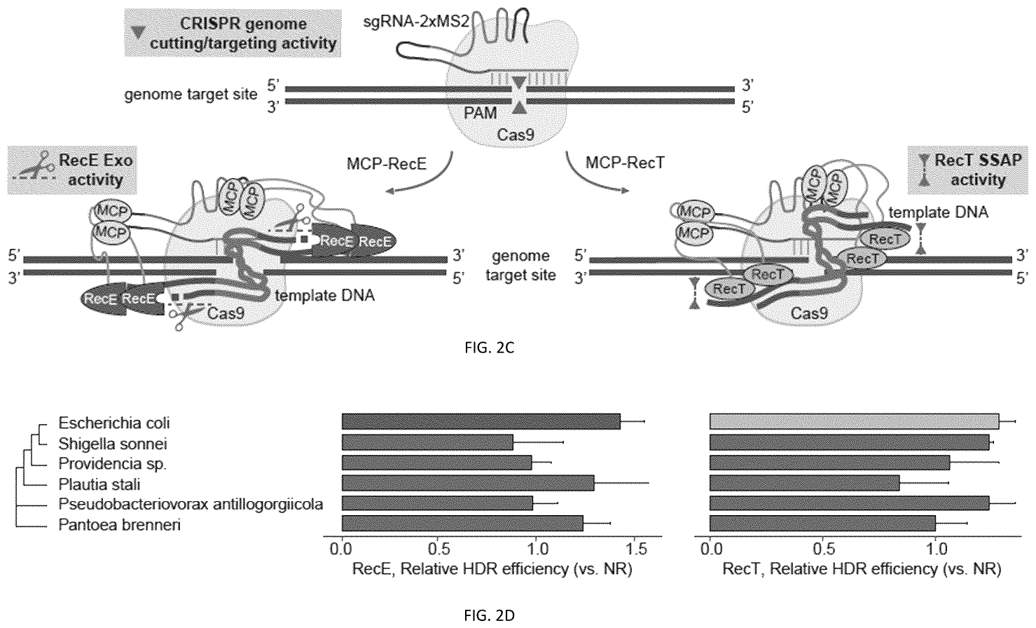

The NCBI protein database was systematically searched for RecE/T homologs. To develop a portable tool, evolutionary relationships and lengths were examined (FIG. 2A). Co-occurrence analysis revealed that most RecE/T systems have only one of the two proteins (FIG. 2B). As prophage integration could be imprecise, the 11% of species harboring both homologs were prioritized as evidence for intact functionality.

The top 12 candidates were codon-optimized and MS2 coat protein (MCP) fusions were constructed to recruit these RecE/T homologs, hereafter termed “recombinator”, to wild-type Streptococcus pyogenes Cas9 (wtCas9) via MS2 RNA aptamers. To understand their respective molecular effects as Exo and SSAP, each was tested independently (FIG. 2C). Initial results revealed Escherichia coli RecE/T proteins (simplified as RecE and RecT) as promising candidates, as determined by genome knock-in assays (FIG. 2D). While RecT is only 269 amino acid (AA) long, RecE was truncated from AA587 (RecE_587) and the carboxy terminus domain (RecE_CTD) based on functional studies (Muyrers, J. P., Genes Dev. (2000); 14, 1971-1982, incorporated herein by reference).

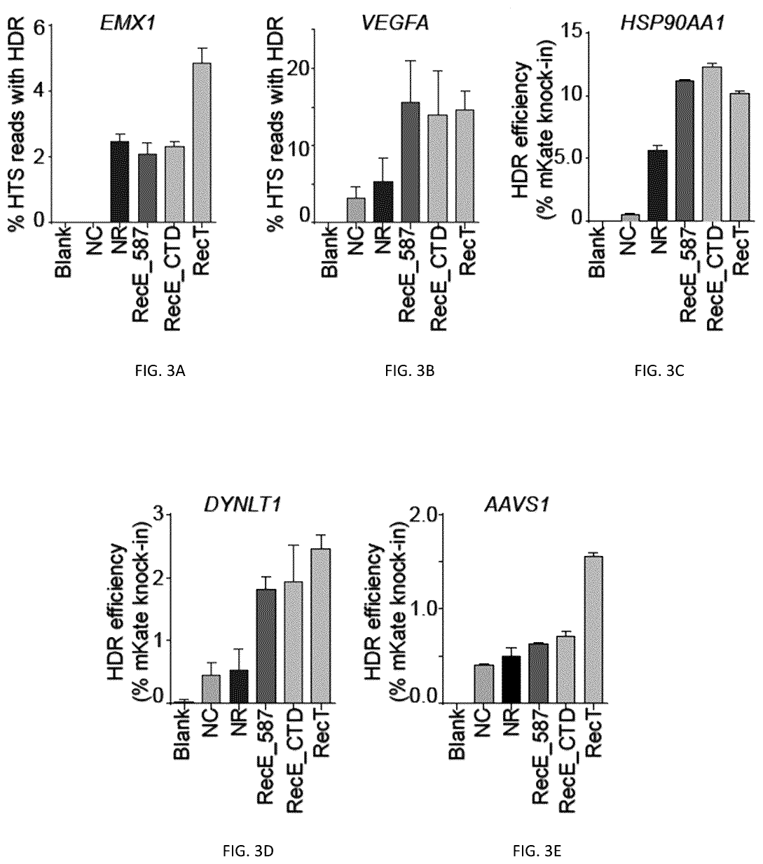

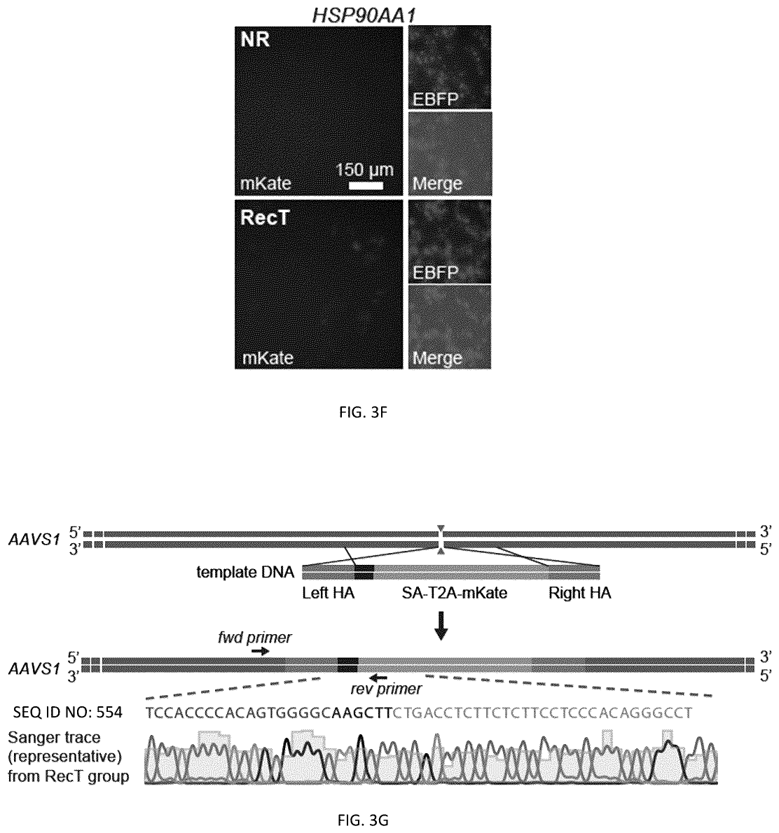

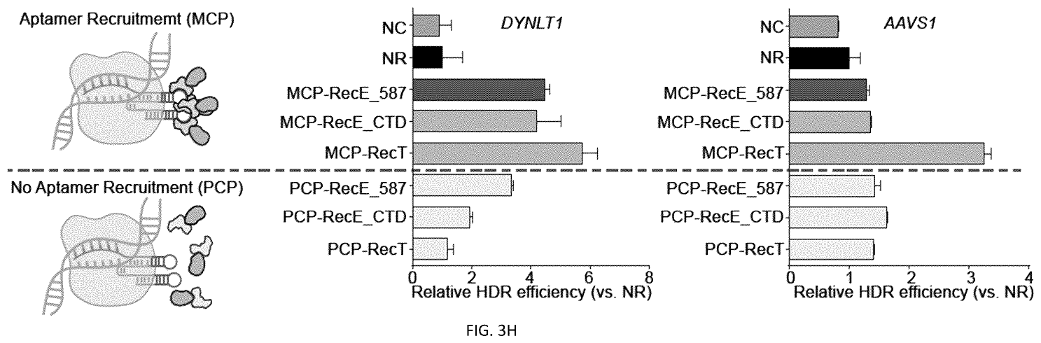

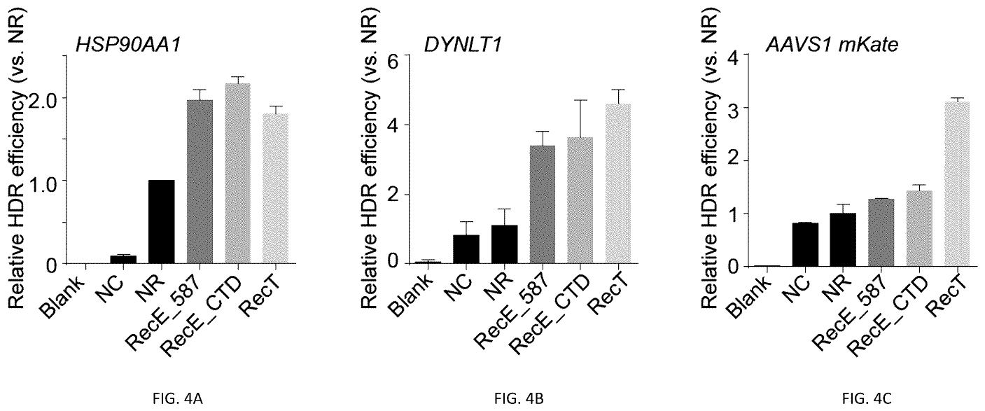

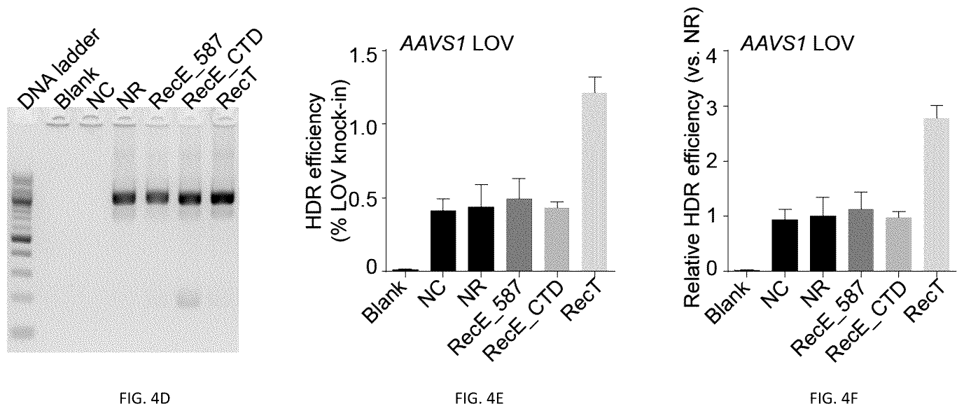

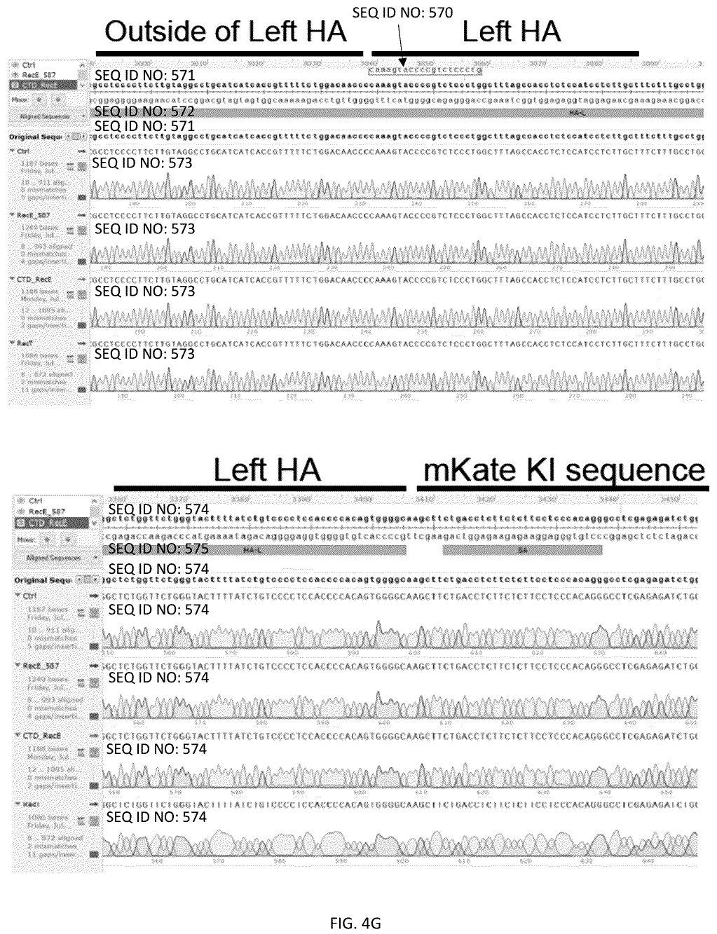

To validate RecE/T recombineering in human cells, homology directed repair (HDR) was measured at five genomic sites with two templates. While the RecE variants (RecE_587, RecE_CTD) demonstrated variable increases in knock-in efficiency, RecT significantly enhanced HDR in all cases, replacing ˜16 bp sequences at EMX1 and VEGFA, and knocking-in ˜1 kb cassette at HSP90AA1, DYNLT1, AAVS1 (FIGS. 3A-E, FIG. 4). These results were verified using imaging (FIG. 3F) and junction sites were sequenced using Sanger sequencing to confirm precise insertion (FIG. 3G, FIG. 4G). To test if these activities are truly sequence-specific, a no-recruitment control with the PP7 coat protein (PCP) that recognizes PP7 aptamers not MS2 aptamers was employed. RecE had activities without recruitment, whereas RecT showed efficiency increases in a recruitment-dependent manner (FIG. 3H). Without being bound by theory, this may be explained by RecE exonuclease activity acting promiscuously (FIG. 2C). The RecE/T recombineering-edit (REDIT) tools was termed as REDITv1, with REDITv1_RecT as the preferred variant.

### Example 3

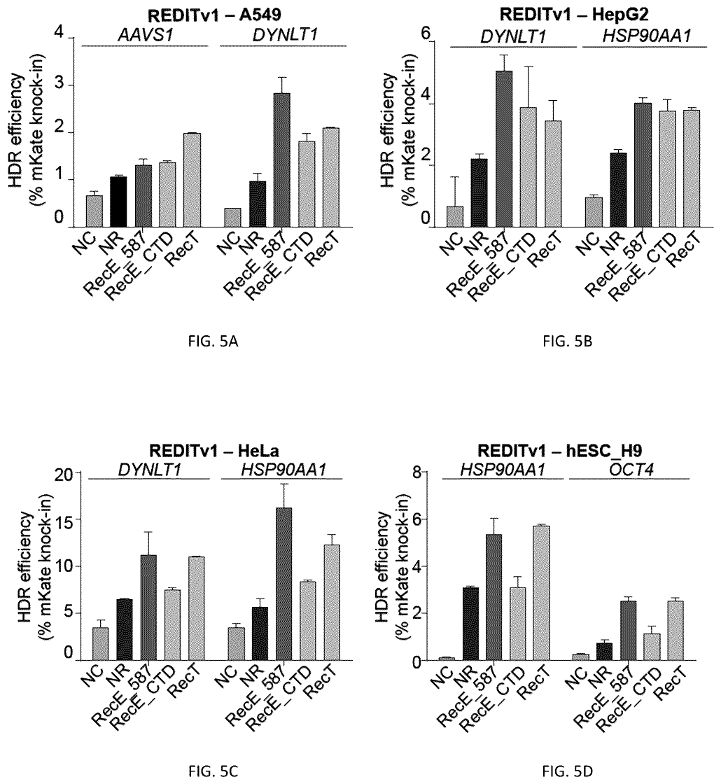

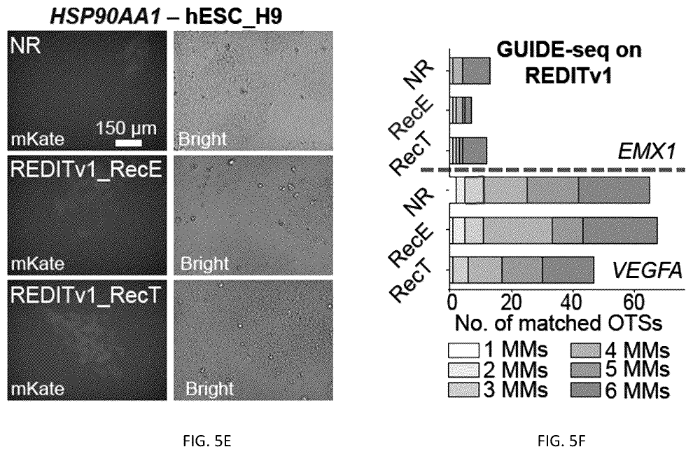

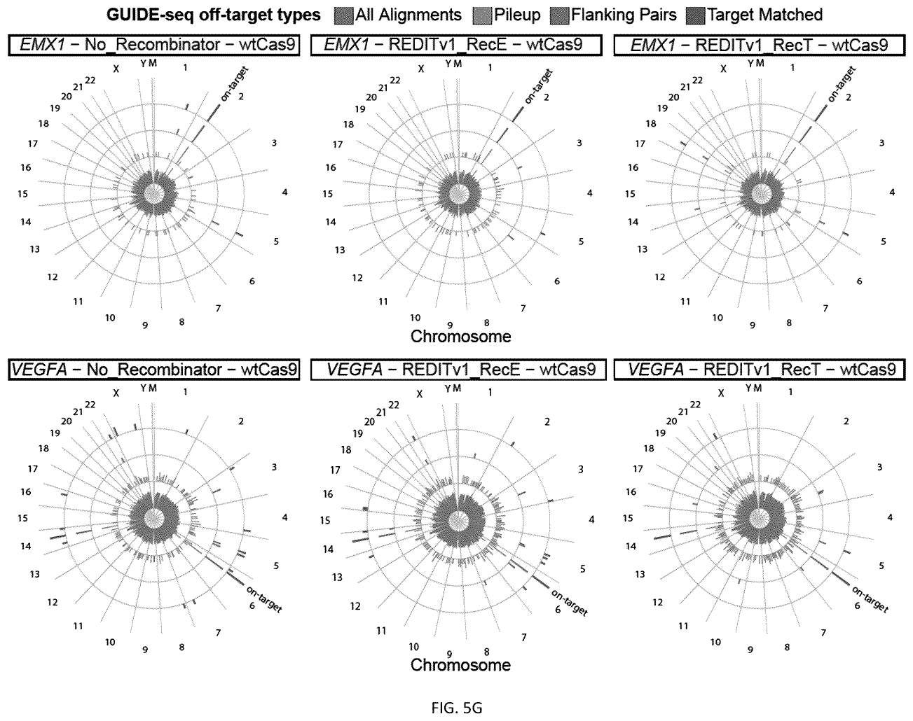

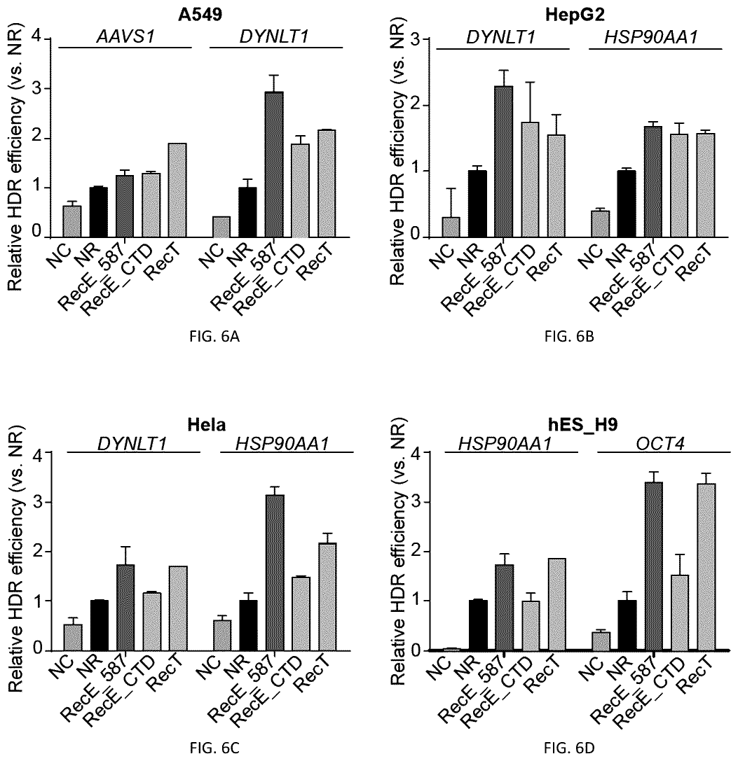

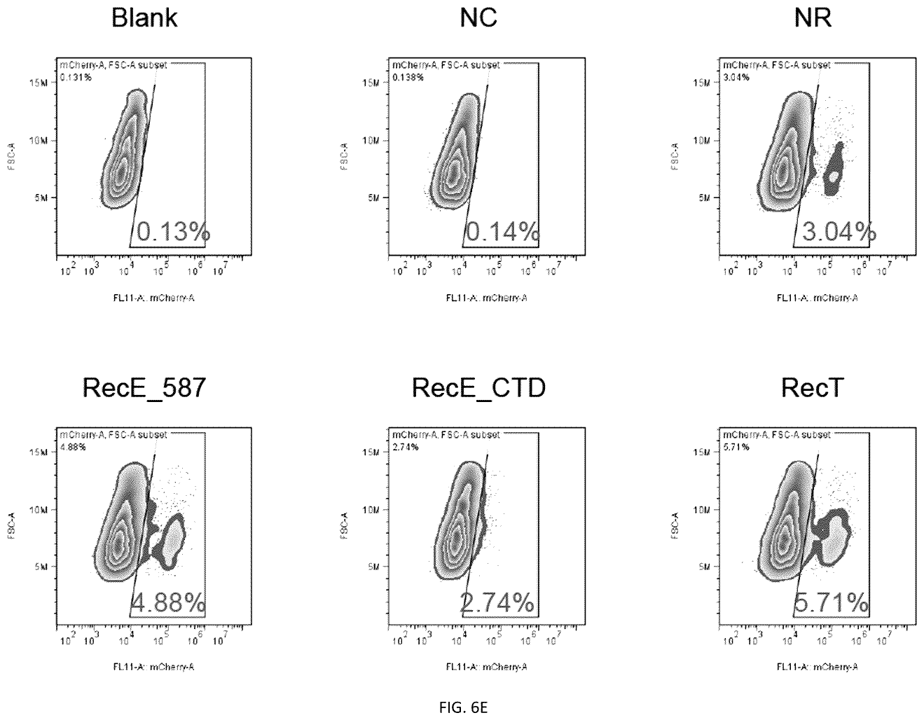

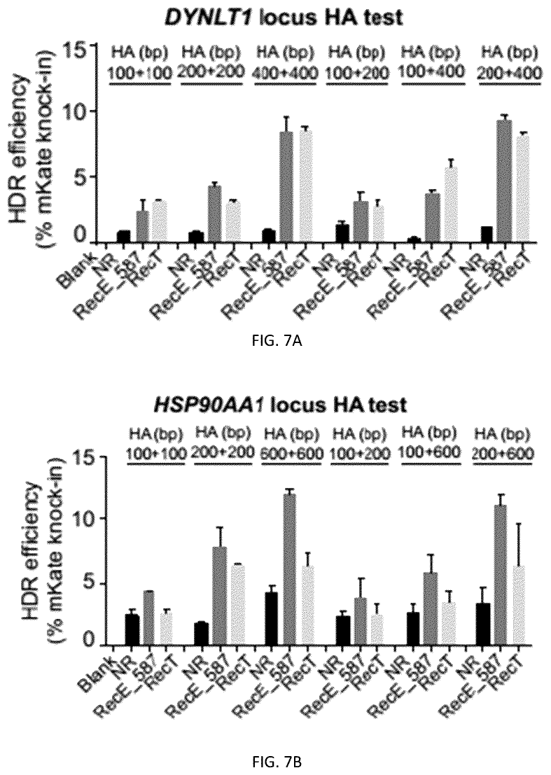

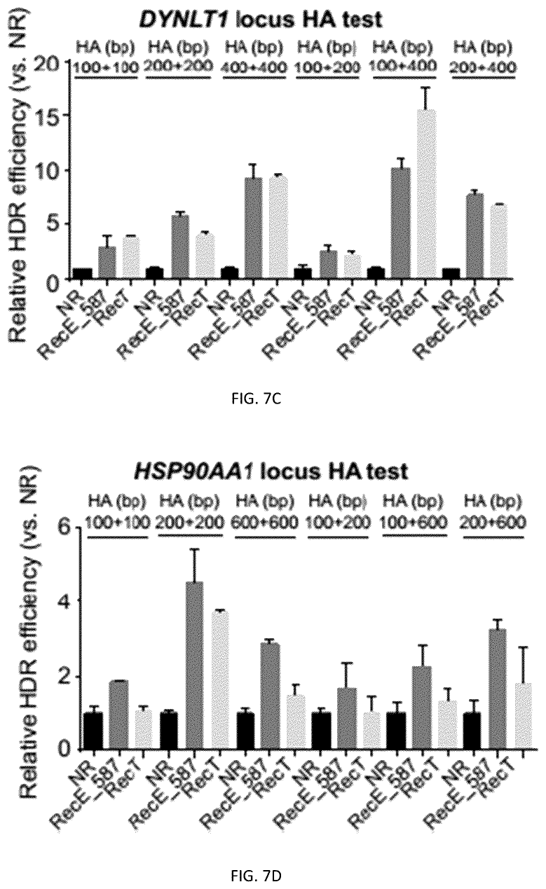

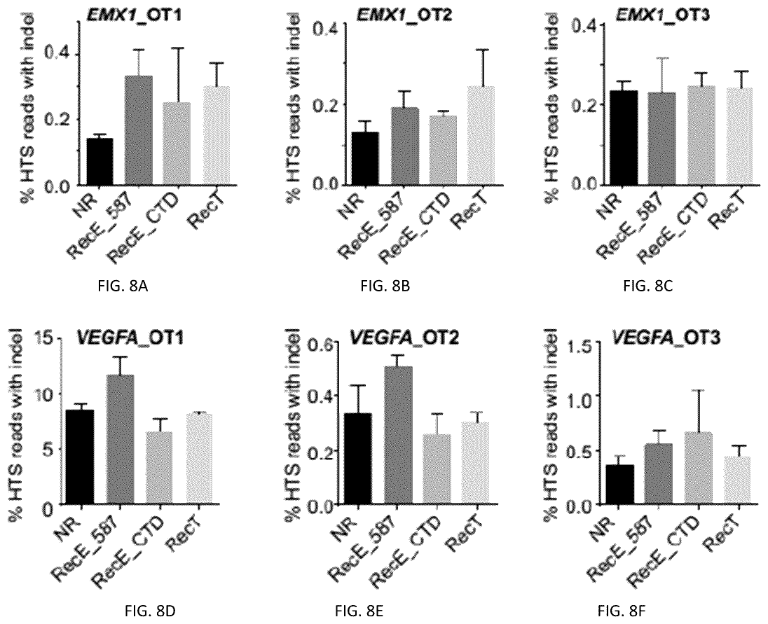

Three tests on REDITv1 were performed to explore: 1) activity across cell types, 2) optimal designs of HDR template, and 3) specificity. REDITv1 activity was robust across multiple genomic sites in HEK, A549, HepG2, and HeLa cells (FIGS. 5A-C, FIGS. 6A-C). Noticeably, in human embryonic stem cells (hESCs), REDITv1 exhibited consistent increases of kilobase knock-in efficiency at HSP90AA1 and OCT4, with up to 3.5-fold improvement relative to Cas9-HDR (FIGS. 5D-E, FIGS. 6D-E). Different template designs were also tested. REDITv1 performed efficient kilobase editing using HA length as short as 200 bp total, with longer HA supporting higher efficiency. It achieved up to 10% efficiency (without selection) for kb-scale knock-in, a 5-fold increase over Cas9-HDR and significantly higher than the 1˜2% typical efficiency (FIG. 7). Lastly, the accuracy of REDITv1 accuracy was determined using deep sequencing of predicted off-target sites (OTSs) and GUIDE-seq. Although REDITv1 did not increase off-target effects, detectable OTSs remained at previously reported sites for EMX1 and VEGFA (FIGS. 5F-G, FIG. 8). In short, REDITv showcased kilobase-scale genome recombineering but retained the off-target issues, with REDITv1_RecT having the highest efficiency.

### Example 4

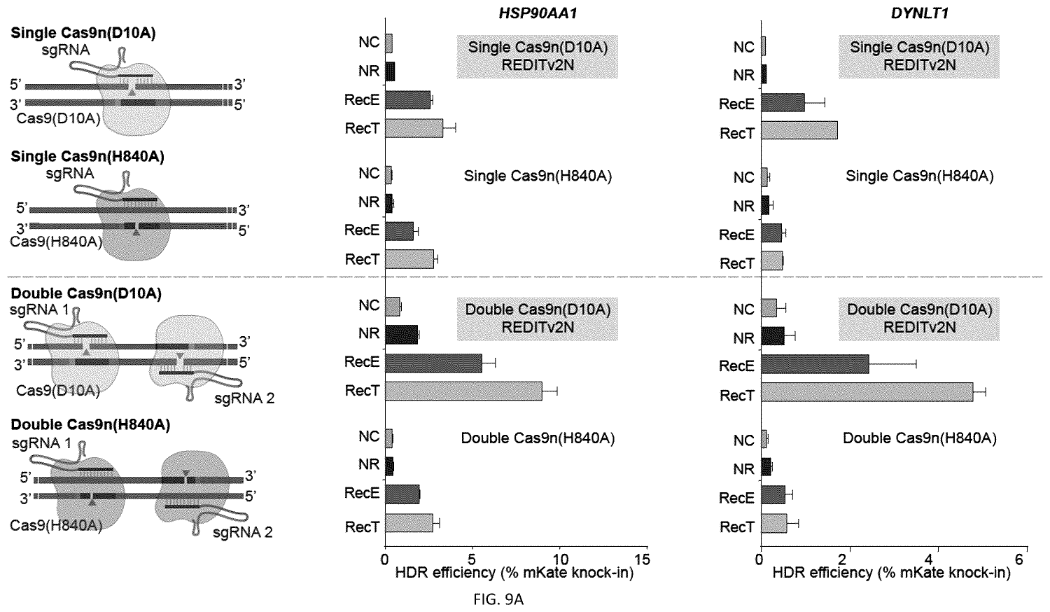

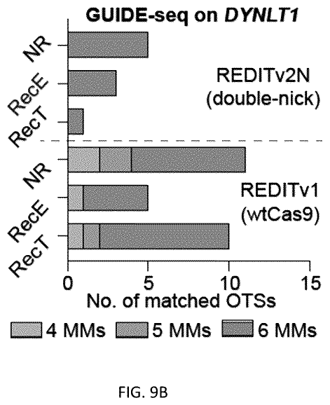

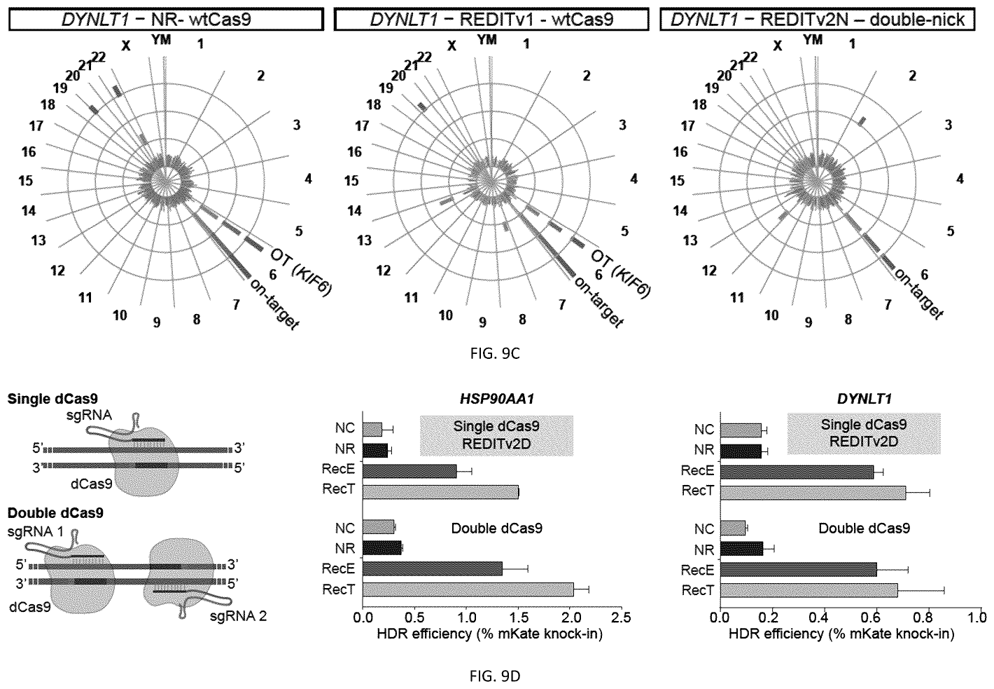

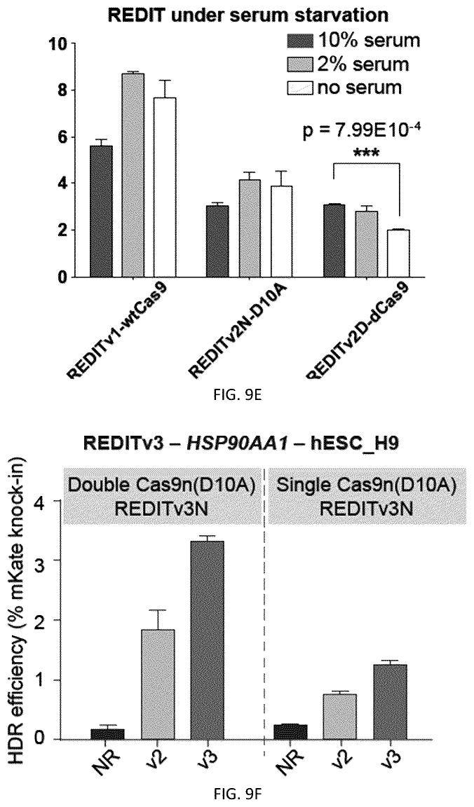

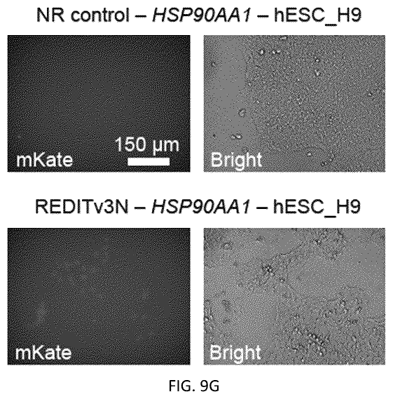

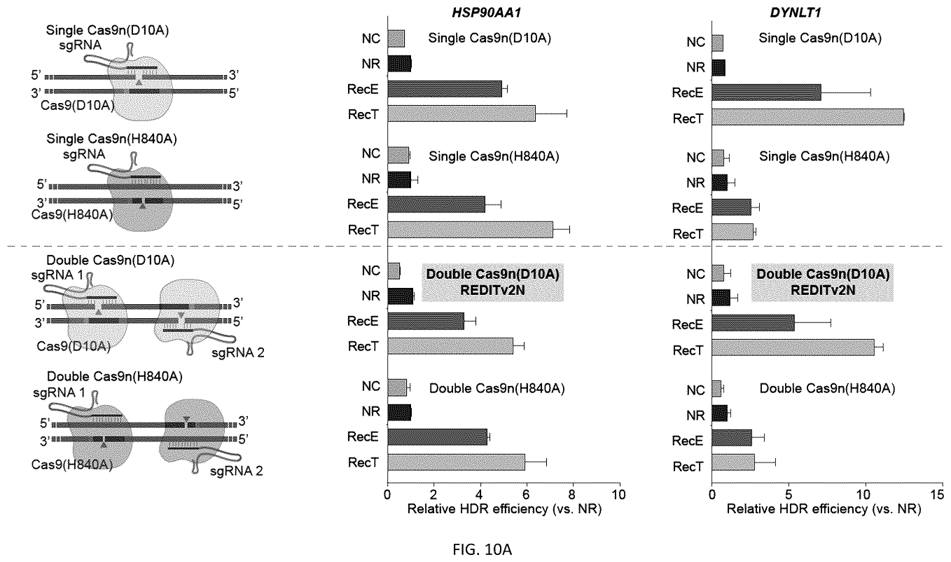

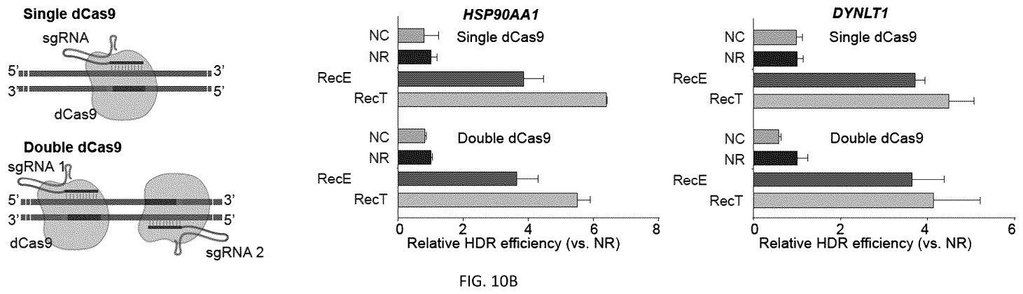

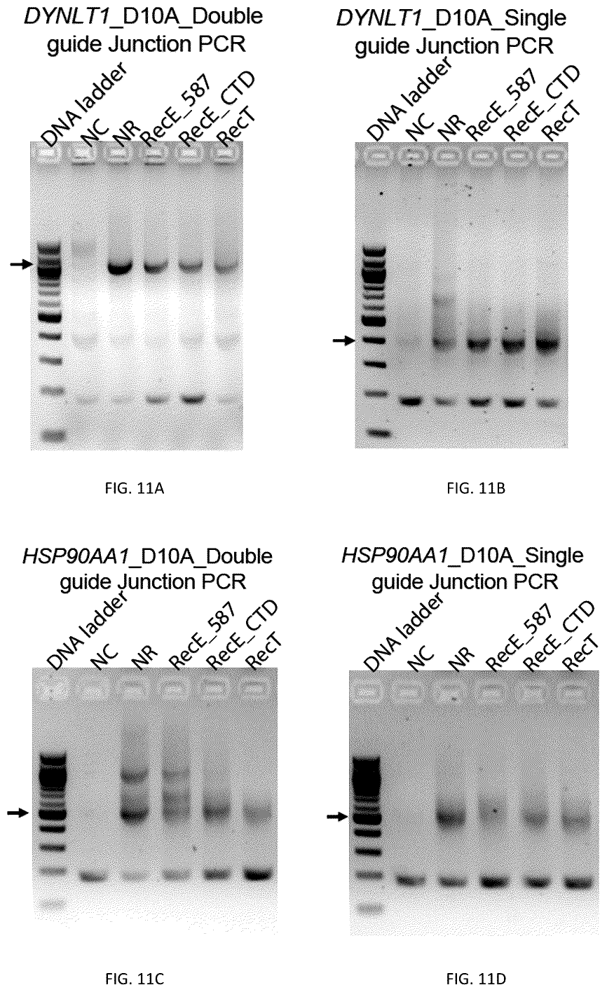

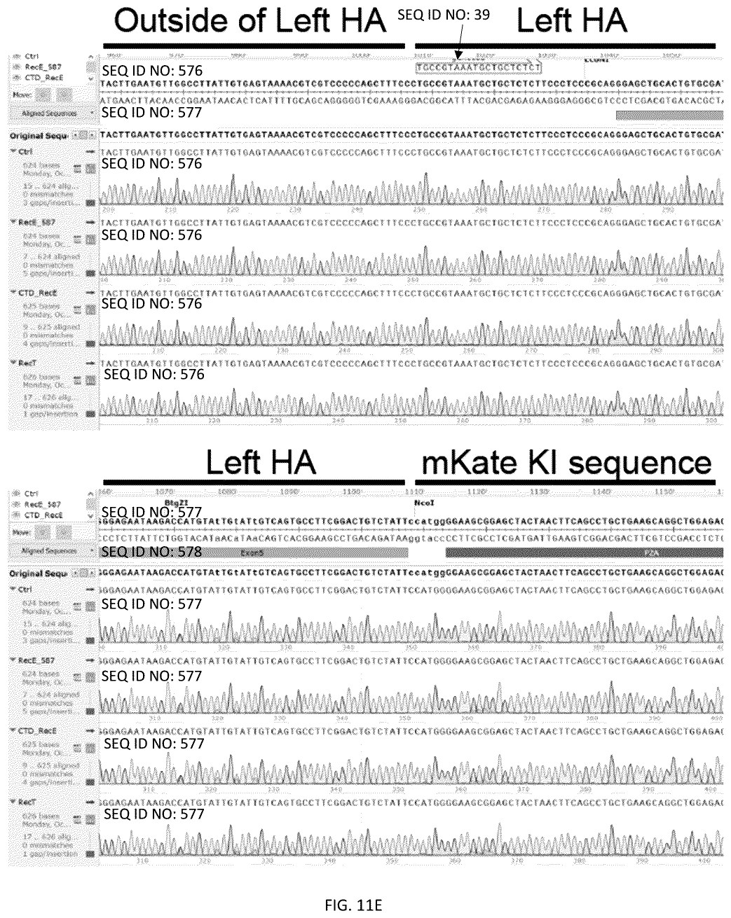

To alleviate unwanted edits, a version of REDIT with non-cutting Cas9 nickases (Cas9n) was assessed. A similar strategy was previously employed (Ran, F. A., et al., Cell (2013), 154:1380-1389, incorporated herein by reference) to address off-target issues but had low HDR efficiency. REDIT was tested to determine if this system could overcome the limitation of endogenous repair and promote nicking-mediated recombination. Indeed, the nickase version demonstrated higher efficiencies, with the best results from Cas9n (D10A) with single- and double-nicking. This Cas9n (D10A) variant was designated REDITv2N (FIG. 9A). A 5%˜10% knock-in without selection was observed using REDITv2N double-nicking, comparable to REDITv1 using wtCas9 (FIG. 9A, FIG. 10A). Junction sequencing confirmed the precision of knock-in for all targets (FIG. 11). This result represented 6- to 10-fold improvement over Cas9n-HDR. Even with single-nicking REDITv2N, a ˜2% efficiency for 1 kb knock-in was observed, a level considerably higher than the 0.46% HDR efficiency in previous report (Cong, L. et al., Science. 339, 819-823, incorporated herein by reference) using regular single-nicking Cas9n and a less-challenging 12-bp knock-in template (FIG. 9A).

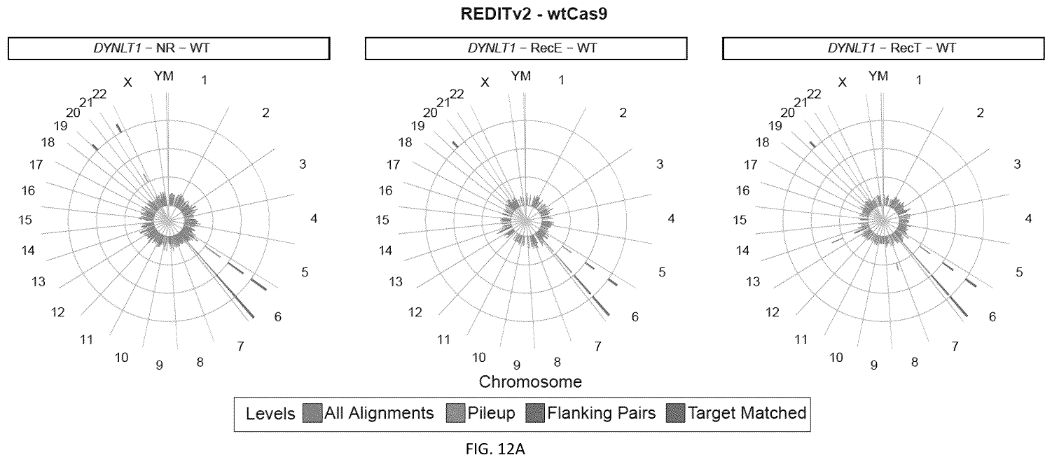

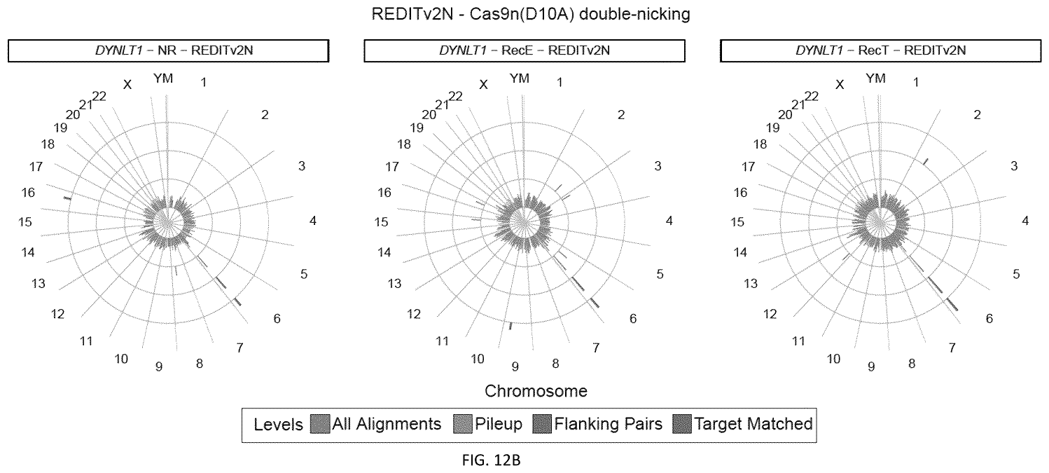

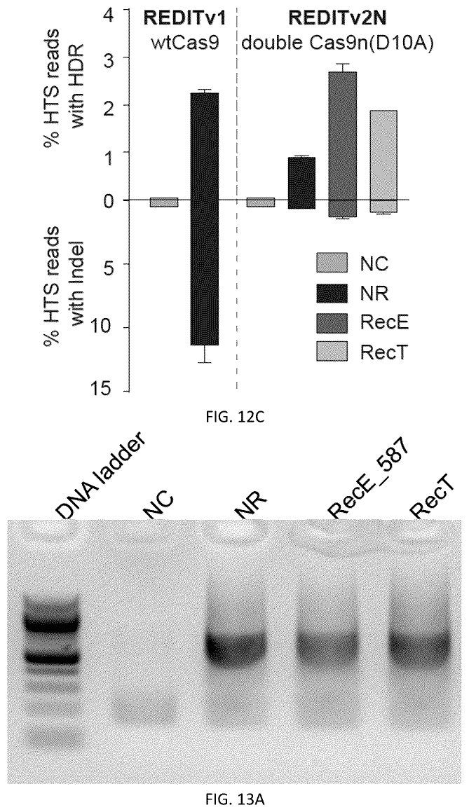

The off-target activity of REDITv2N was investigated using GUIDE-seq. Results showed minimal off-target cleavage and a reduction of OTSs by ˜90% compared to REDITv1 (FIG. 9B). Specifically, for DYNLT1-targeting guides, the most abundant KIF6 OTS was significantly enriched in REDITv1 group but disappeared when using REDITv2N (FIG. 9C). REDITv2N was highly accurate (FIGS. 9B-C, FIG. 12).

Another byproduct of HDR editing is on-target insertion-deletions (indels). They could drastically lower yields of gene-editing, especially for long sequences. Indel formation was measured in an EMX1 knock-in experiment using deep sequencing. REDITv2N increased HDR to the same efficiency as its counterpart using wtCas9 (FIG. 12C, top), with a reduction of unwanted on-target indels by 92% (FIG. 12C, bottom).

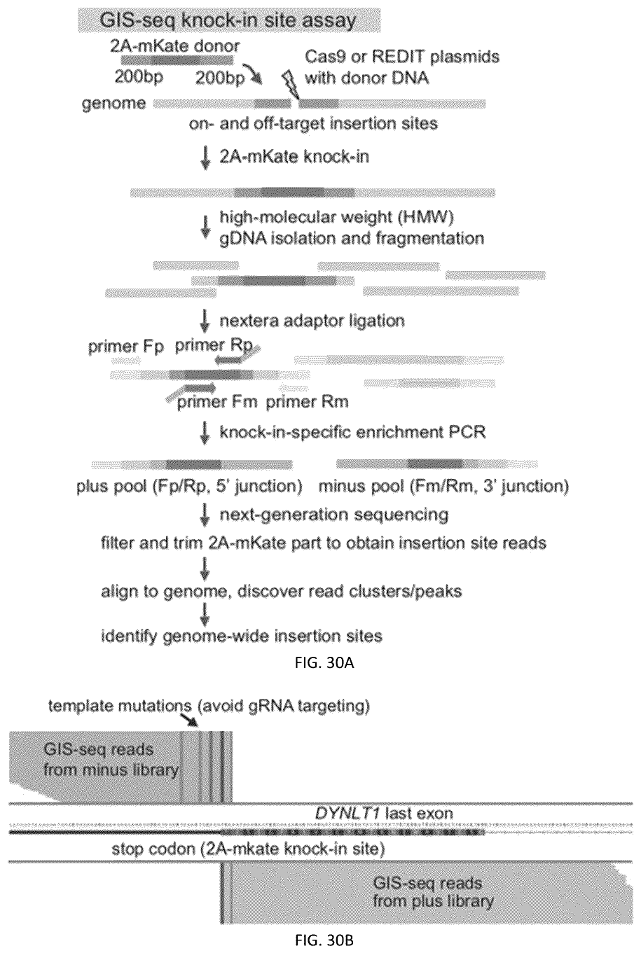

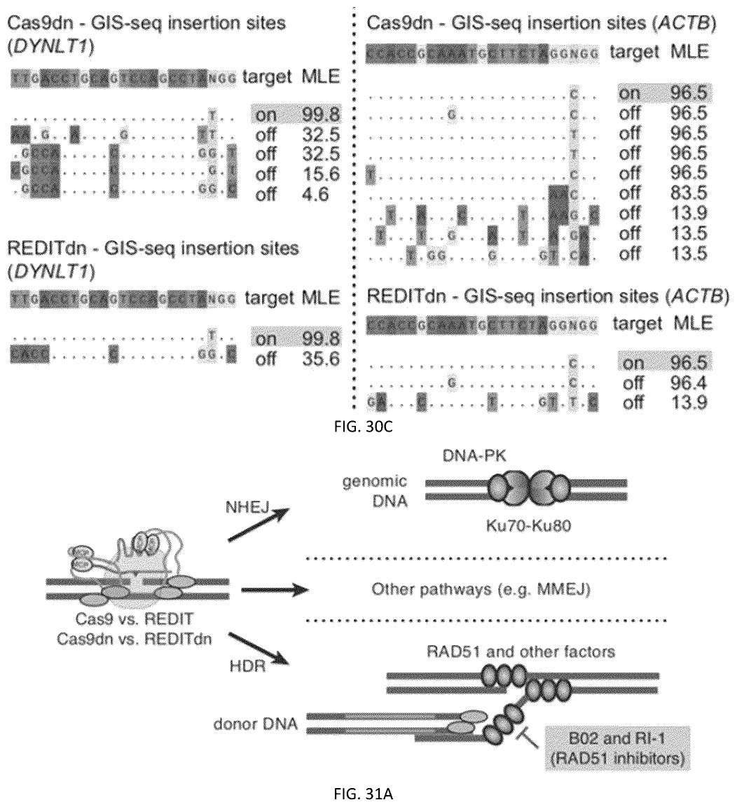

Concepts from GUIDE-seq, LAM-PCR, and TLA were used to develop an NGS-based assay to identify genome-wide insertion sites (GIS), or GIS-seq (FIG. 30A). Using GIS-seq, NGS read clusters/peaks representing knock-in insertion sites were obtained (FIG. 30B), showing representative reads from the on-target site). GIS-seq was applied to DYNLT1 and ACTB loci to measure the knock-in accuracy. Sequencing results indicated that, when considering sites with high confidence based on maximum likelihood estimation, REDIT had less off-target insertion sites identified compared with Cas9 (FIG. 30C). Together, the clonal Sanger sequencing of knock-in junctions (FIGS. 9C and 12), GUIDE-seq analysis (FIG. 9B), and GIS seq results (FIGS. 30A-30C) indicated that REDIT can be an efficient method with the ability to insert kilobase-length sequences with less unwanted editing events.

### Example 5

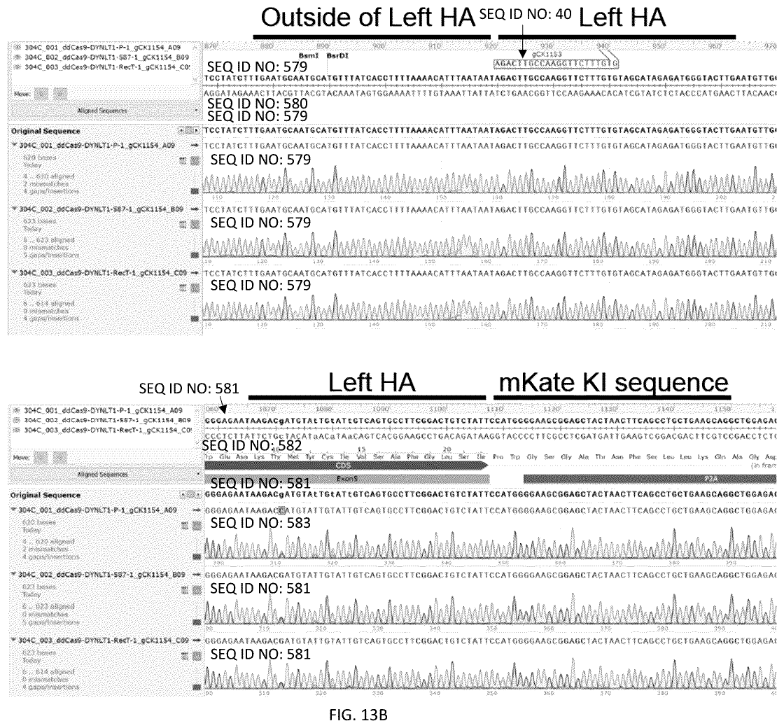

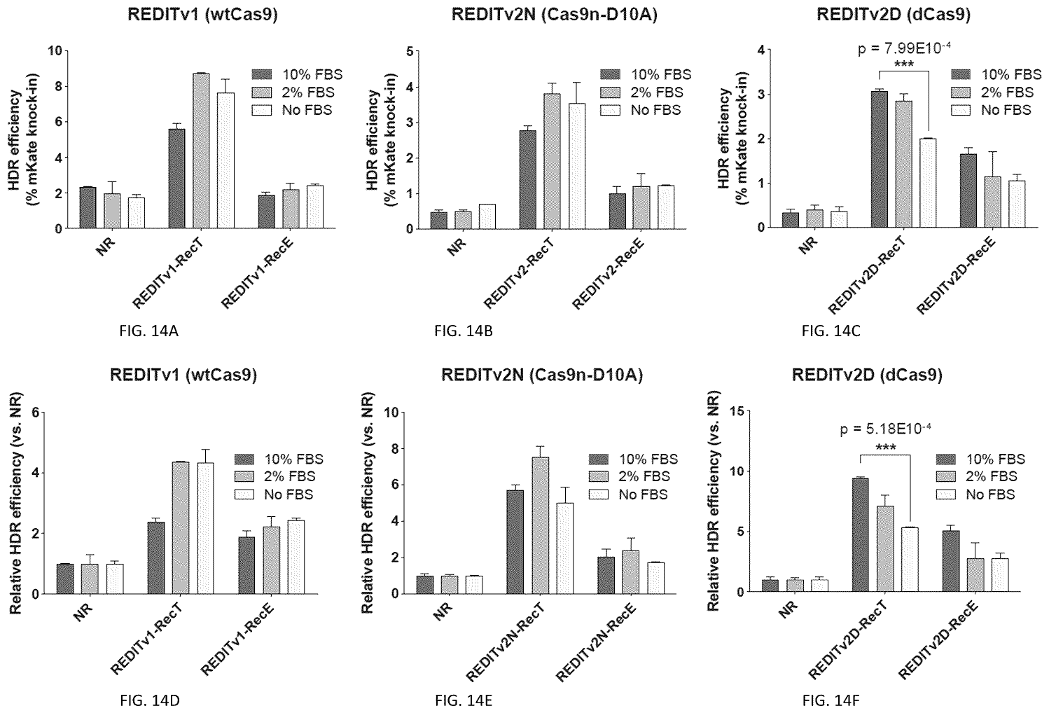

REDIT was examined for long sequence editing ability in the absence of any nicking/cutting of the target DNA. Remarkably, when using catalytically dead Cas9 (dCas9) to construct REDITv2D, an exact genomic knock-in of a kilobase cassette was observed in human cells (FIG. 9D, top, FIG. 13). While REDITv2D has lower efficiency than REDITv2N, it achieved programmable DNA-damage-free editing at kilobase-scale with 1˜2% efficiency and no selection (FIG. 9D, FIG. 10B). It was hypothesized that two processes could be contributing to the REDITv2D recombineering. One possibility was via dCas9 unwinding. If dCas9 could unwind DNA as it induces sequence-specific formation of loop, a double-binding with two dCas9s would be expected to promote genome accessibility to RecE/T. However, a significant increase upon delivering two guide RNAs was not observed (FIG. 9D, bottom). Another possibility was that the unwinding of DNA during cell cycle permitted RecE/T to access the target region mediated by dCas9 binding. A 1 kb knock-in was performed with different REDIT tools at varying serum levels (10% regular, 2% reduced, and no serum). As serum starvation arrests cell proliferation, the results indicated that the cell cycle correlated positively with REDITv2D recombineering (FIG. 9E). Upon no-serum treatment, HDR efficiency only dropped in REDITv2D (dCas9) group, whereas REDITv1 (wtCas9) and REDITv2N (D10A) were not affected (FIG. 9E, FIG. 14), supporting that DNA unwinding permitted RecE/T to access the target region.

### Example 6

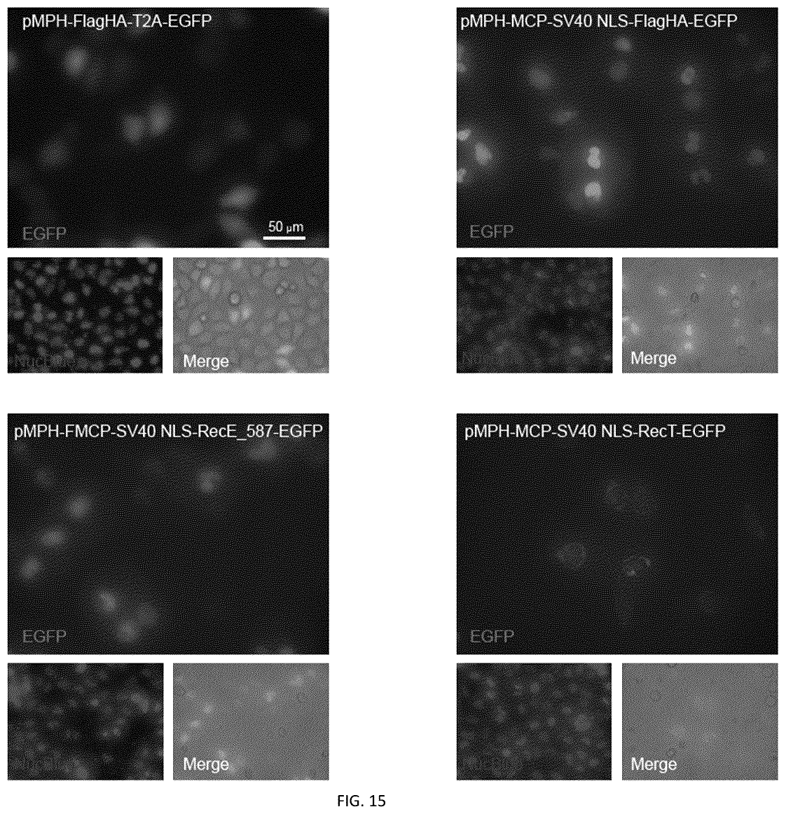

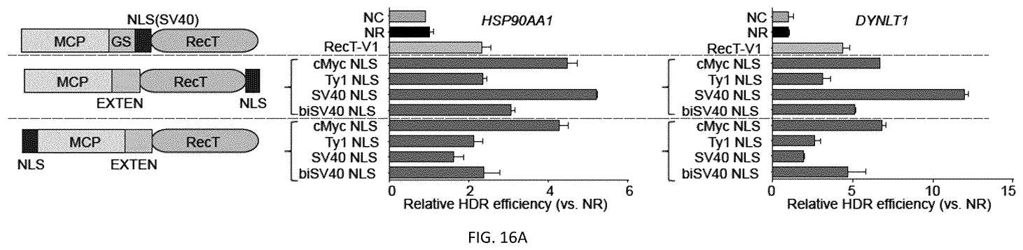

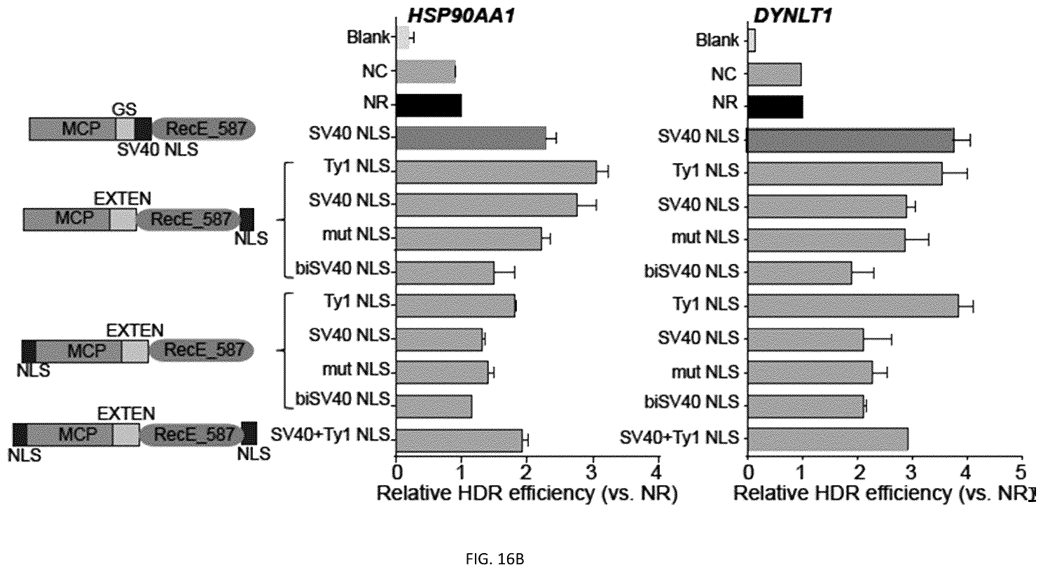

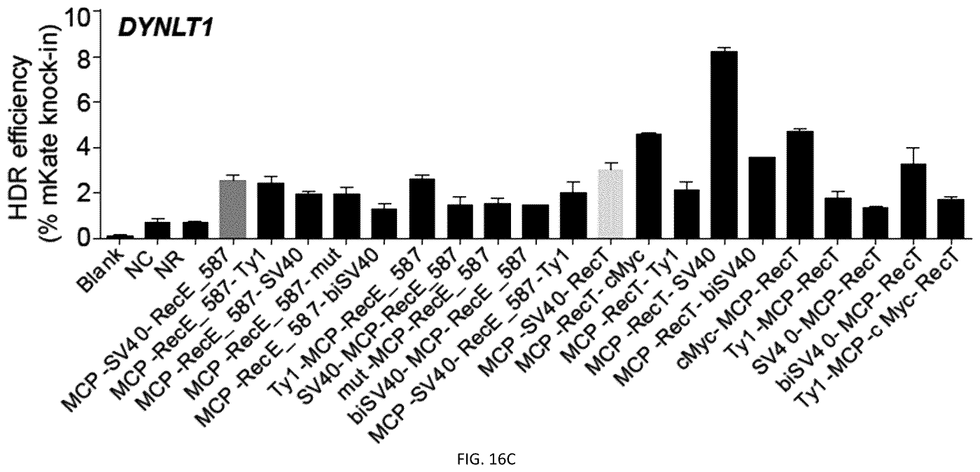

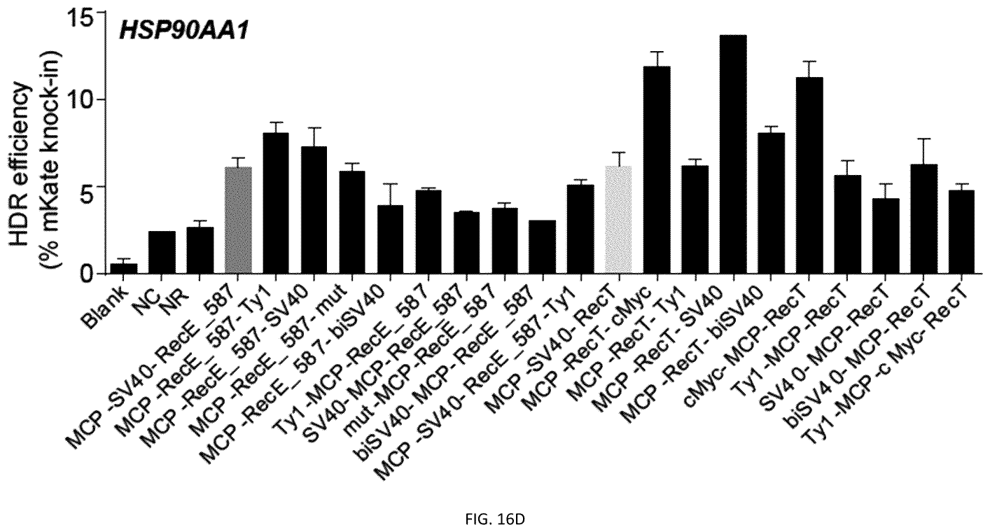

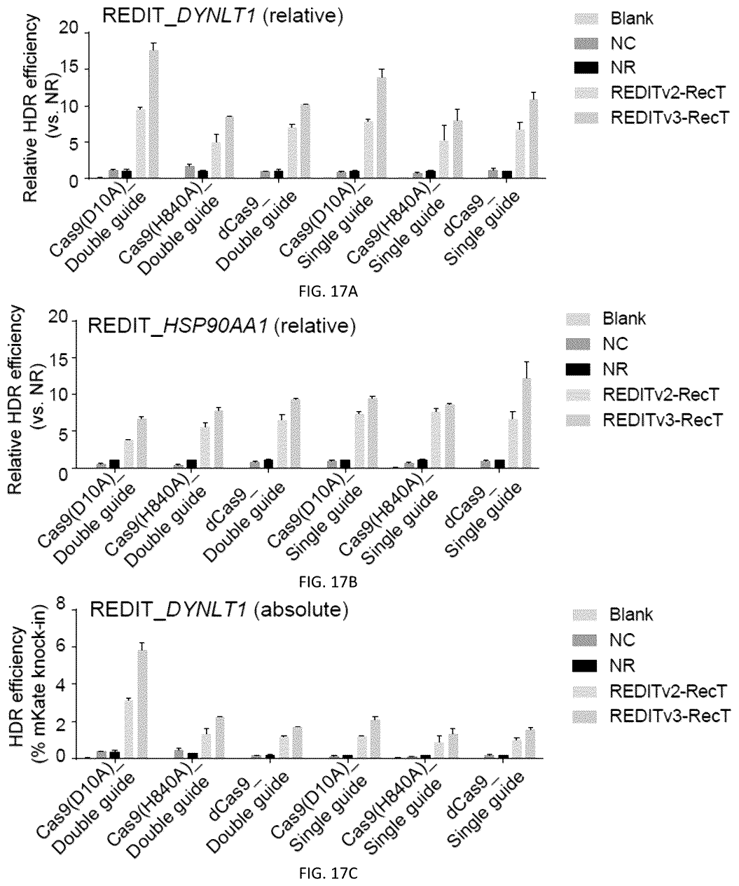

Microscopy analysis revealed incomplete nuclei-targeting of REDITv1, particularly REDITv1_RecT (FIG. 15). Hence, different designs of protein linkers and nuclear localization signals (NLSs) were tested (FIG. 15A). The extended XTEN-linker with C-terminal SV40-NLS was identified as a preferred configuration, termed REDITv3 (FIG. 16). REDITv3 further achieved a 2- to 3-fold increase of HDR efficiencies over REDITv2 across genome targets and Cas9 variants (wtCas9, Cas9n, dCas9) (FIG. 17).

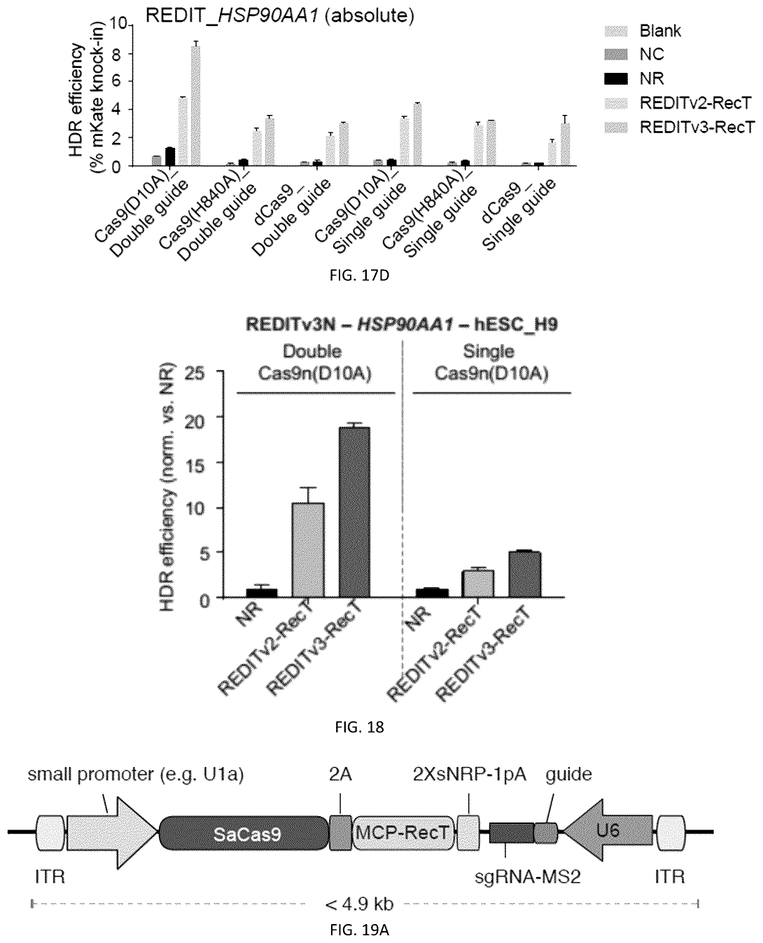

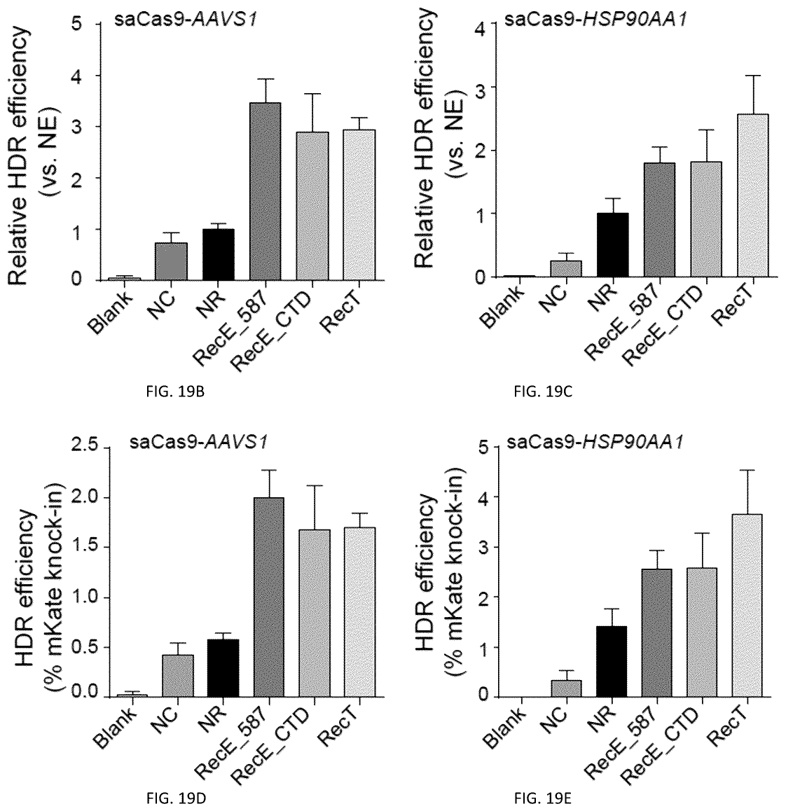

Finally, REDITv3 was utilized in hESCs to engineer kilobase knock-in alleles in human stem cells. REDITv3N single- and double-nicking designs resulted in 5-fold and 20-fold increased HDR efficiencies over no-recombinator controls, respectively (FIG. 9F). The efficacy and fidelity were confirmed via a combination of assays described for previous REDIT versions (FIGS. 9F-G, FIG. 18). Additionally, REDITv3 works effectively with Staphylococcus aureus Cas9 (SaCas9), a compact CRISPR system suitable for in vivo delivery (FIG. 19).

### Example 7

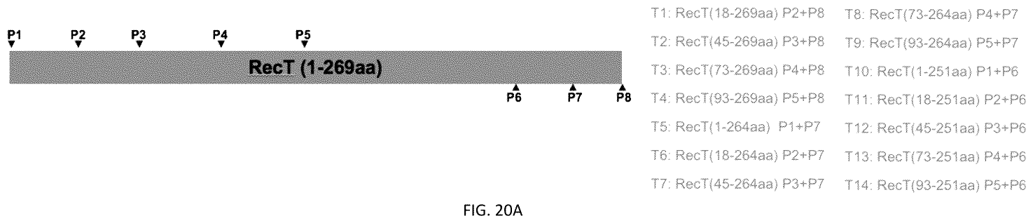

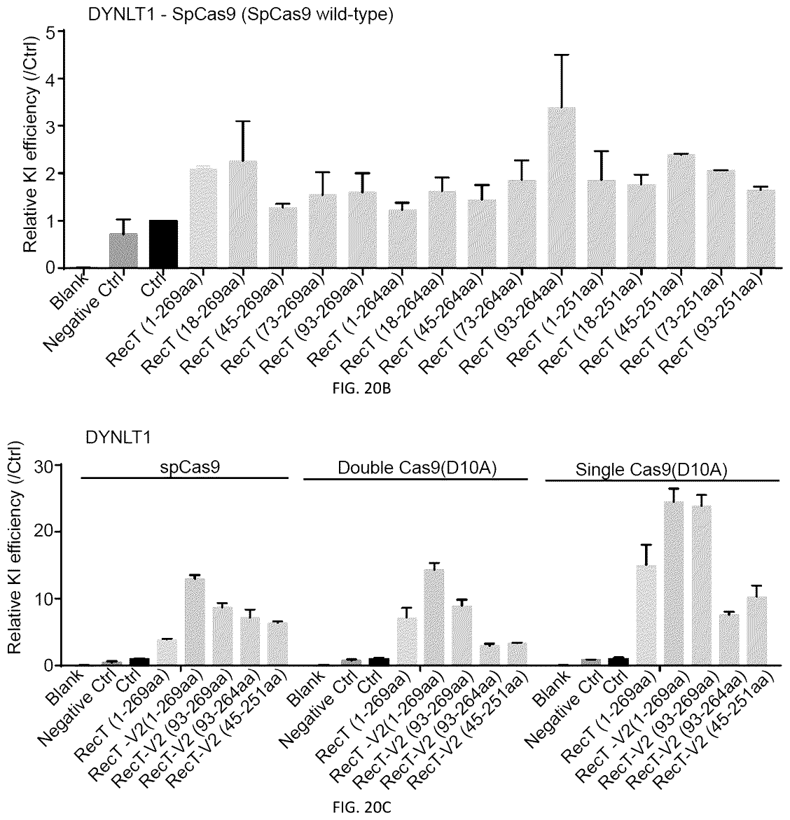

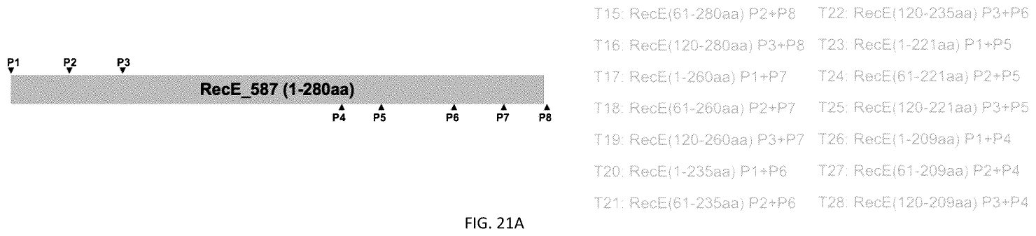

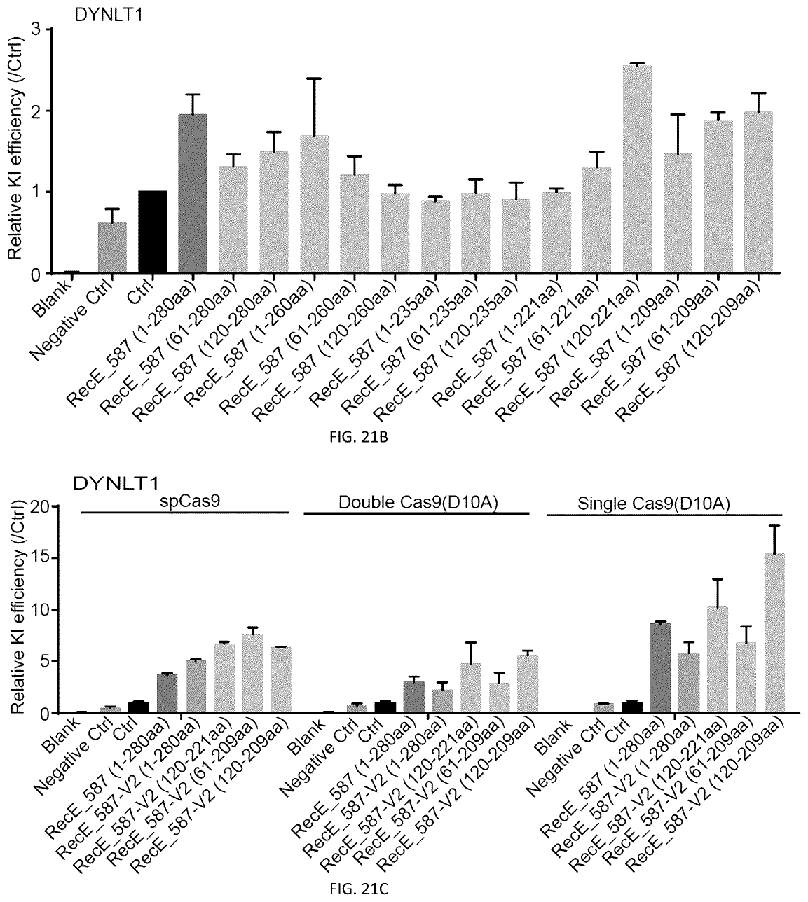

To further investigate RecT and RecE_587 variants, both RecT and RecE_587 were truncated at various lengths as shown in FIG. 20A and FIG. 21A, respectively. The resulting efficiencies were measured using an mKate knock-in assay, with both wildtype SpCas9 and Cas9n (D10A) with single- and double-nicking at the DYNLT1 locus (FIGS. 20B-C and FIGS. 21B-C, respectively). Efficiencies of the no recombination group are shown as the control.

The truncated versions of both RecT and RecE_587 retained significant recombineering activity when used with different Cas9s. In particular, compared with the full-length RecT (1-269aa), the new truncated versions such as RecT (93-264aa) are over 30% smaller yet they preserved essentially the full activities of RecT in stimulating recombination in eukaryotic cells. Similarly, compared with the full-length RecE (1-280aa), truncated versions such as RecE_587 (120-221aa) and RecE_587 (120-209aa) are over 60% smaller but still retained high recombination activities in human cells. These truncated versions demonstrated the potential to further engineer minimal-functional recombineering enzymes using RecE and RecT protein variants, but also provide valuable compact recombineering tools for human genome editing that is ideal for in vitro, ex vivo, and in vivo delivery given their small size.

Overall, REDIT harnessed the specificity of CRISPR genome-targeting with the efficiency of RecE/RecT recombineering. The disclosed high-efficiency, low-error system makes a powerful addition to existing CRISPR toolkits. The balanced efficiency and accuracy of REDITv3N makes it an attractive therapeutic option for knock-in of large cassette in immune and stem cells.

### Example 8

The reconstructed RecE and RecT phylogenetic trees with eukaryotic recombination enzymes from yeast and human (FIGS. 1A and 1B) show the evolutionary distance of the proteins based on sequence homology. The dotted boxes indicate the full-length E. coli RecB and E. coli RecE protein. The catalytic core domain of E. coli RecB and E. coli RecE protein (solid boxes) was used for the comparison. The gene-editing activities of these families of recombineering proteins were measured using the MS2-MCP recruitment system, where sgRNA bearing MS2 stem-loop is used with recombineering proteins fused to the MCP protein via peptide linker and with nuclear-localization signals.

Three exonuclease proteins were used: the exonuclease from phage Lambda, the RecE587 core domain of E. coli RecE protein, and the exonuclease (gene name gp6) from phage T7 (FIG. 22A). The gene-editing activity was measured using mKate knock-in assay at genomic loci (DYNLT1 and HSP90AA1).

Similar measurements were made testing the genome editing efficiencies of three single-strand DNA annealing proteins (SSAPs) from the same three species of microbes as the exonucleases, namely Bet protein from phage Lambda, RecT protein from E. coli, and SSAP (gene name gp2.5) from phage T7 (FIG. 22B).

From these results, the genome recombineering activities of all three major family of phage/microbial recombination systems was systematically measured and validated in eukaryotic cells (lambda phage exonuclease and beta proteins; E. coli prophase RecE and RecT proteins, T7 phage exonuclease gp6 and single-strand binding gp2.5 proteins). All six proteins from three systems achieved efficient gene editing to knock-in kilobase-long sequences into mammalian genome across two genomic loci. Overall, the exonucleases showed ˜3-fold higher recombination efficiency (up to 4% mKate genome knock-in) when compared with no-recombinator controls. The single-strand annealing proteins (SSAP) showed higher activities, with 4-fold to 8-fold higher gene-editing activities over the control groups. This demonstrated the general applicability and validity that microbial recombination proteins in the exonuclease and SSAP families could be engineered via the Cas9-based fusion protein system to achieve highly efficient genome recombination in mammalian cells.

### Example 9

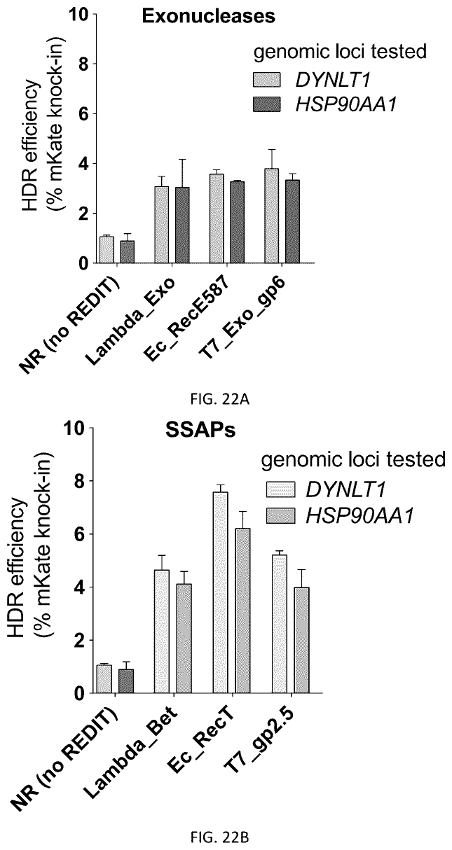

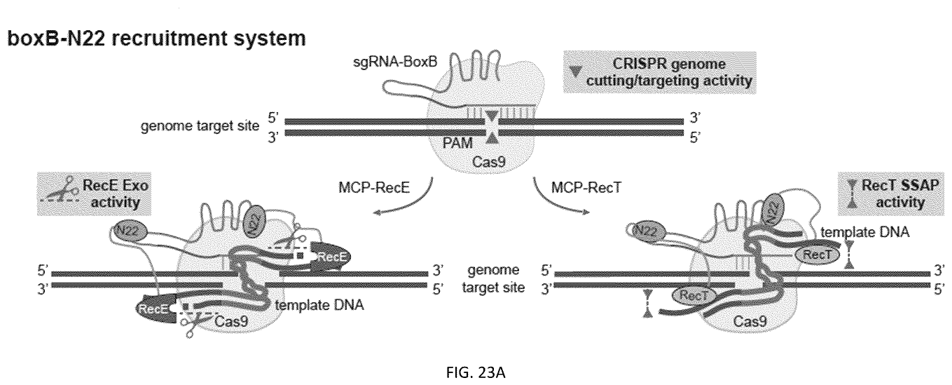

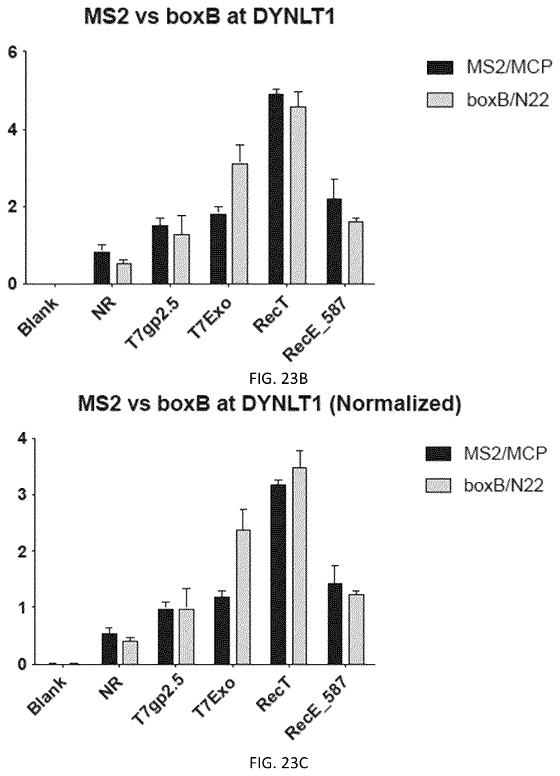

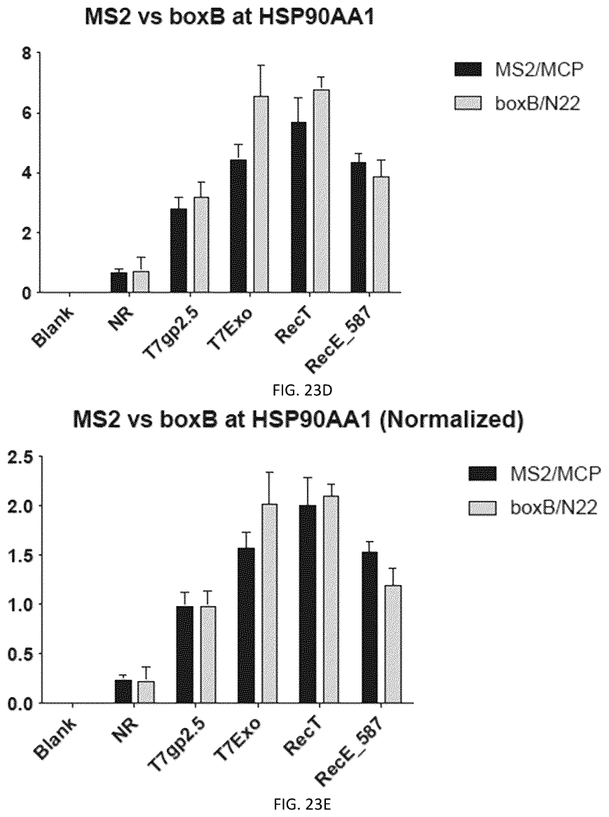

In order to demonstrate the generalizability of REDIT protein design, alternative recruitment systems were developed and tested. For a more compact REDIT system, the REDIT recombinator proteins were fused to N22 peptide and at the same time the sgRNA included boxB, the short cognizant sequence of N22 peptide, replacing MCP within the sgRNA (FIG. 23A). This boxB-N22 system demonstrated comparable editing efficiencies at the two genomic sites tested as shown in FIGS. 23B-23E with side-by-side comparisons of the MS2-MCP recruitment system.

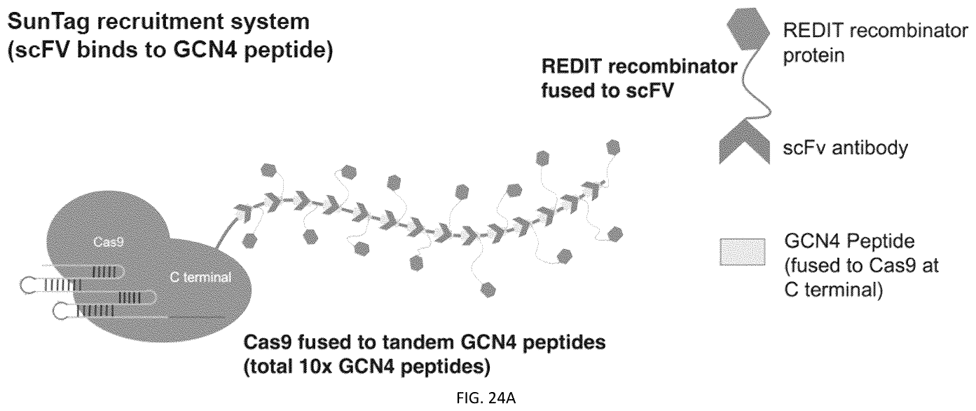

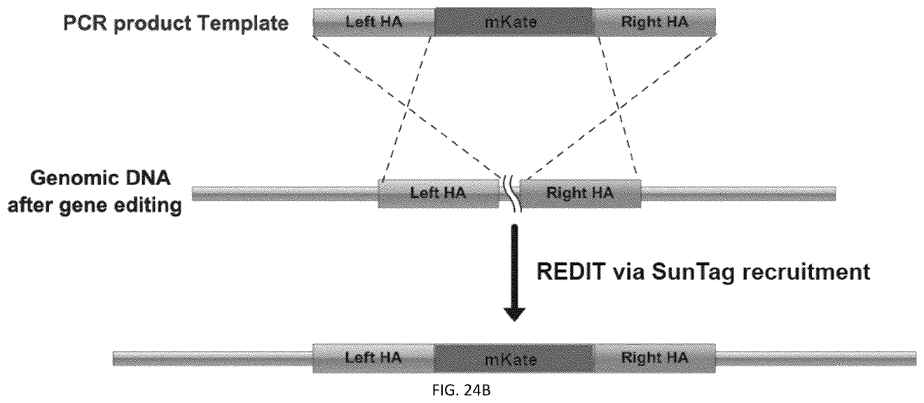

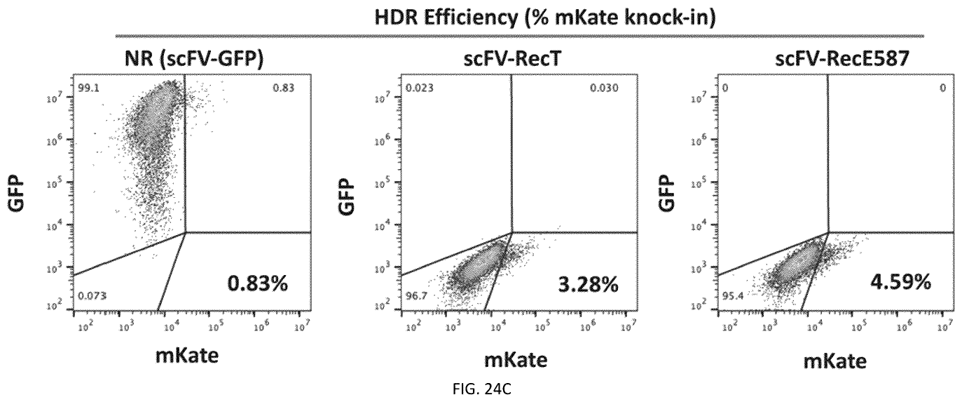

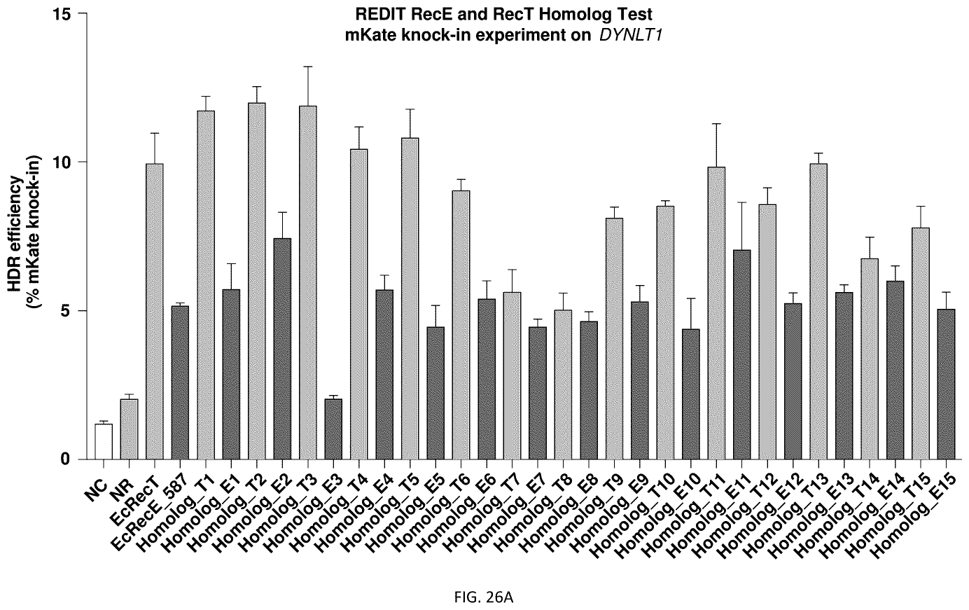

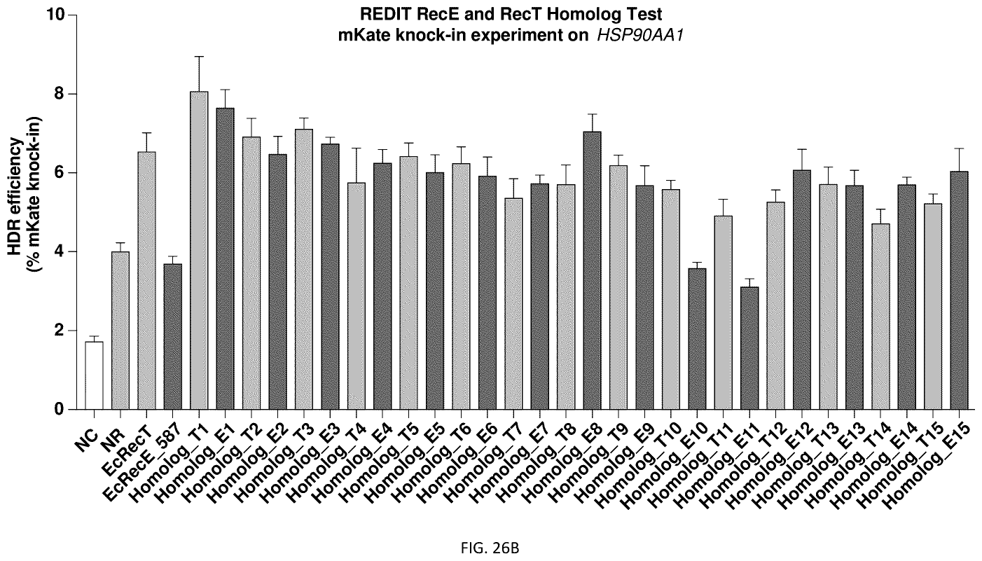

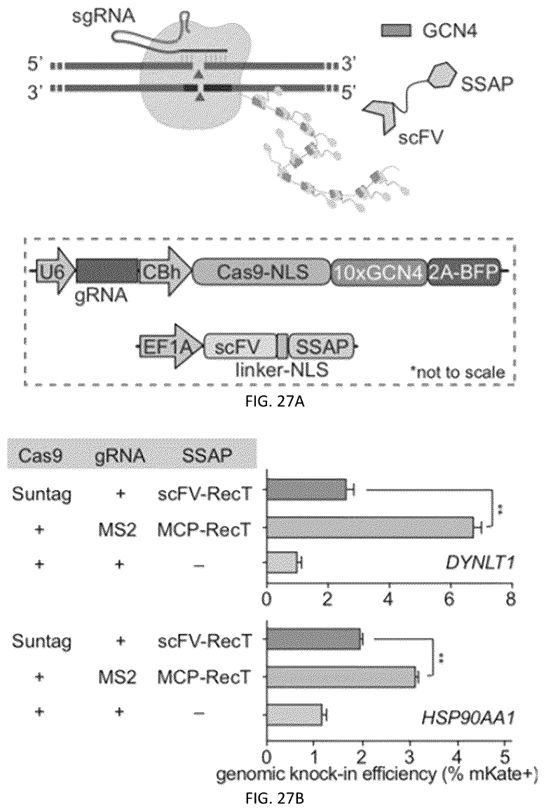

A REDIT system using SunTag recruitment, a protein-based recruitment system, was developed (FIGS. 24A and 27A). Because SunTag is based on fusion protein design, the sgRNA or guideRNAs are the same as wild-type CRISPR system. Specifically, the REDIT recombinator proteins were fused to scFV antibody peptide (replacing MCP), and the GCN4 peptide was fused in tandem fashion (10 copies of GCN4 peptide separated by linkers) to the Cas9 protein. Thus, the scFV-REDIT could be recruited to the Cas9 complex via GCN4's affinity to scFV.

mKate knock-in experiments (FIGS. 24B and 27B) were used to measure the editing efficiencies at the DYNLT1 locus and the HSP90AA1 locus, respectively. This SunTag-based REDIT system demonstrated significant increase of gene-editing knock-in efficiency at the DYNLT1 genomic sites tested. In addition, the SunTag design significantly increased HRD efficiencies to ˜2-fold better than Cas9 but did not achieve increases as high as the MS2-aptamer.

### Example 10

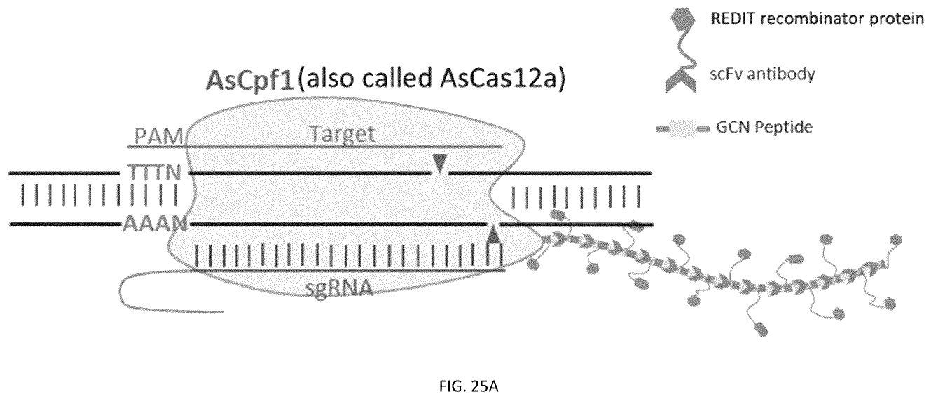

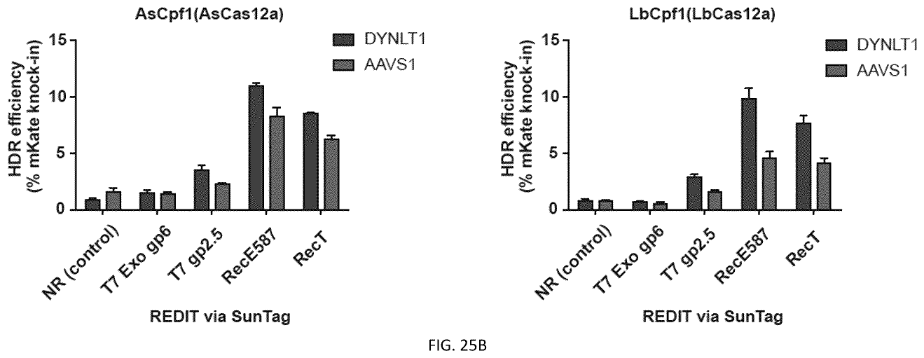

In order to demonstrate the generalizability of REDIT protein design and develop versatile REDIT system applicable to a range of CRISPR enzymes, Cpf1/Cas12a based REDIT system using the SunTag recruitment design was developed (FIG. 25A). Two different Cpf1/Cas12a proteins were tested (Lachnospiraceae bacterium ND2006, LbCpf1 and Acidaminococcus sp. BV3L6) using the mKate knock-in assay as previously shown (FIG. 25B).

These results showed that the microbial recombination proteins (exonuclease and single-strand annealing proteins) could be engineered using alternative designs such as the SunTag recruitment system to perform genome editing in eukaryotic cells. These protein-based recruitment system does not require the usage of RNA aptamers or RNA-binding proteins, instead, they took advantage of fusion protein domains directly connecting to the CRISPR enzymes to recruit REDIT proteins.

In addition to the flexibility in recruitment system design, these results using Cpf1/Cas 12a-type CRISPR enzymes also demonstrated the general adaptability of REDIT proteins to various CRISPR systems for genome recombination. Cpf1/Cas12a enzymes have different catalytic residues and DNA-recognition mechanisms from the Cas9 enzymes. Hence, the REDIT recombination proteins (exonucleases and single-strand annealing proteins) could function independent from the specific choices of the CRISPR enzyme components (Cas9, Cpf1/Cas12a, and others). This proved the generalizability of the REDIT system and open up possibility to use additional CRISPR enzymes (known and unknown) as components of REDIT system to achieve accurate genome editing in eukaryotic cells.

### Example 11

15 different species of microbes having RecE/RecT proteins were selected for a screen of various RecE and RecT proteins across the microbial kingdom (Table 3). Each protein was codon-optimized and synthesized. As previously described for E. coli RecE/RecT based REDIT systems, each protein was fused via E-XTEN linker to the MCP protein with additional nuclear localization signal. mKate knock-in gene-editing assay was used to measure efficiencies at DYNLT1 locus (FIG. 26A, Table 4) and HSP90AA1 locus (FIG. 26B, Table 4). The homologs demonstrated the ability to enable and enhance precision gene-editing.

TABLE 3
RecE and RecT protein homologs

| Homolog | Source | Protein |
|---|---|---|
| T1 | Pantoea stewartii | RecT |
| E1 | Pantoea stewartii | RecE |
| T2 | Pantoea brenneri | RecT |
| E2 | Pantoea brenneri | RecE |
| T3 | Pantoea dispersa | RecT |
| E3 | Pantoea dispersa | RecE |
| T4 | Type-F symbiont of Plautia stali | RecT |
| E4 | Type-F symbiont of Plautia stali | RecE |
| T5 | Providencia stuartii | RecT |
| E5 | Providencia stuartii | RecE |
| T6 | Providencia sp. MGF014 | RecT |
| E6 | Providencia sp. MGF014 | RecE |
| T7 | Providencia alcalifaciens DSM 30120 | RecT |
| E7 | Providencia alcalifaciens DSM 30120 | RecE |
| T8 | Shewanella putrefaciens | RecT |
| E8 | Shewanella putrefaciens | RecE |
| T9 | Bacillus sp. MUM 116 | RecT |
| E9 | Bacillus sp. MUM 116 | RecE |
| T10 | Shigella sonnei | RecT |
| E10 | Shigella sonnei | RecE |
| T11 | Salmonella enterica | RecT |
| E11 | Salmonella enterica | RecE |
| T12 | Acetobacter | RecT |
| E12 | Acetobacter | RecE |
| T13 | Salmonella enterica subsp. enterica | RecT |
| | serovar Javiana str. 10721 | |
| E13 | Salmonella enterica subsp. enterica | RecE |
| | serovar Javiana str. 10721 | |
| T14 | Pseudobacteriovorax antillogorgiicola | RecT |
| E14 | Pseudobacteriovorax antillogorgiicola | RecE |
| T15 | Photobacterium sp. JCM 19050 | RecT |
| E15 | Photobacterium sp. JCM 19050 | RecE |

TABLE 4
mKate Knock-In Gene-Editing Efficiencies

| | DYNLT1 | | HSP90AA1 | |
|---|---|---|---|---|
| | Mean | | Mean | |
| | mKate+ (%) | SEM | mKate+ (%) | SEM |
| NC | 1.2100 | 0.0802 | 1.7333 | 0.1245 |
| NR | 2.0500 | 0.1442 | 4.0100 | 0.2166 |
| EcRecE_587 | 5.1767 | 0.0897 | 3.7067 | 0.1784 |
| EcRecT | 9.9467 | 1.0143 | 6.5467 | 0.4646 |
| Homolog_T1 | 11.7333 | 0.4667 | 8.0733 | 0.8752 |
| Homolog_E1 | 5.7333 | 0.8503 | 7.6567 | 0.4556 |
| Homolog_T2 | 12.0000 | 0.5292 | 6.9233 | 0.4594 |
| Homolog_E2 | 7.4533 | 0.8553 | 6.4867 | 0.4359 |
| Homolog_T3 | 11.9000 | 1.3013 | 7.1200 | 0.2730 |
| Homolog_E3 | 2.0533 | 0.1020 | 6.7467 | 0.1565 |
| Homolog_T4 | 10.4433 | 0.7331 | 5.7567 | 0.8704 |
| Homolog_E4 | 5.7200 | 0.4744 | 6.2567 | 0.3339 |
| Homolog_T5 | 10.8267 | 0.9445 | 6.4300 | 0.3262 |
| Homolog_E5 | 4.4667 | 0.7116 | 6.0233 | 0.4366 |
| Homolog_T6 | 9.0533 | 0.3548 | 6.2500 | 0.4100 |
| Homolog_E6 | 5.4100 | 0.5981 | 5.9300 | 0.4708 |
| Homolog_T7 | 5.6467 | 0.7383 | 5.3700 | 0.4795 |
| Homolog_E7 | 4.4733 | 0.2444 | 5.7367 | 0.2105 |
| Homolog_T8 | 5.0400 | 0.5599 | 5.7133 | 0.4886 |
| Homolog_E8 | 4.6567 | 0.3088 | 7.0533 | 0.4388 |
| Homolog_T9 | 8.1300 | 0.3523 | 6.2000 | 0.2511 |
| Homolog_E9 | 5.3233 | 0.5233 | 5.6900 | 0.4903 |
| Homolog_T10 | 8.5333 | 0.1601 | 5.5900 | 0.2237 |
| Homolog_E10 | 4.4000 | 1.0149 | 3.5900 | 0.1442 |
| Homolog_T11 | 9.8467 | 1.4374 | 4.9233 | 0.4074 |
| Homolog_E11 | 7.0567 | 1.5872 | 3.1167 | 0.2010 |
| Homolog_T12 | 8.5900 | 0.5401 | 5.2733 | 0.2935 |
| Homolog_E12 | 5.2633 | 0.3374 | 6.0800 | 0.5164 |
| Homolog_T13 | 9.9567 | 0.3324 | 5.7200 | 0.4267 |
| Homolog_E13 | 5.6333 | 0.2360 | 5.6900 | 0.3729 |
| Homolog_T14 | 6.7700 | 0.7022 | 4.7200 | 0.3612 |
| Homolog_E14 | 6.0167 | 0.4890 | 5.7100 | 0.1793 |
| Homolog_T15 | 7.8033 | 0.7075 | 5.2333 | 0.2302 |
| Homolog_E15 | 5.0700 | 0.5543 | 6.0500 | 0.5696 |

### Example 12

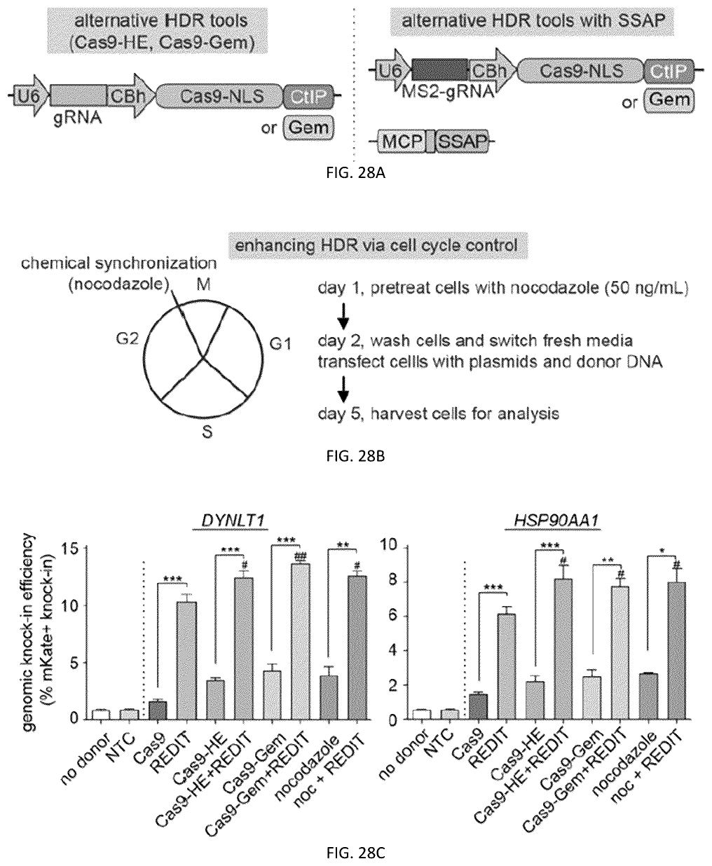

Next, to benchmark the RecT-based REDIT design, it was compared with three categories of existing HDR-enhancing tools (FIGS. 28A and 28B): DNA repair enzyme CtIP fusion with the Cas9 (Cas9-HE), a fusion of the functional domain (amino acids 1 to 110) of human Geminin protein with the Cas9 (Cas9-Gem), and a small-molecule enhancers of HDR via cell cycle control, Nocodazole. Across endogenous targets tested, the RecT-based REDIT design had favorable performance compared with three alternative strategies (FIG. 3C). Furthermore, the RecT-based REDIT design, which putatively acted through activity independently from the other approaches, may synergize with existing methods. To test this hypothesis, RecT-based REDIT design was combined with three different approaches (conveniently through the MS2-aptamer) (FIG. 28A, right). The RecT-based REDIT design could indeed further enhance the HDR-promoting activities of the tested tools (FIG. 28C).

### Example 13

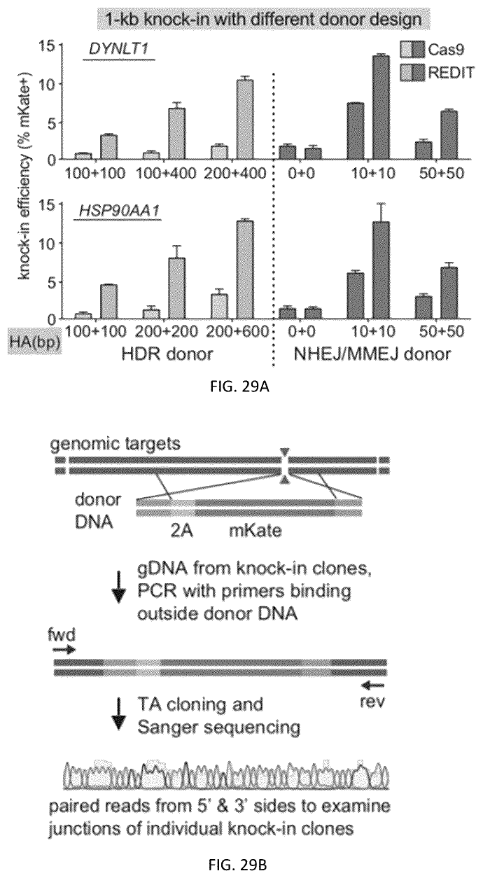

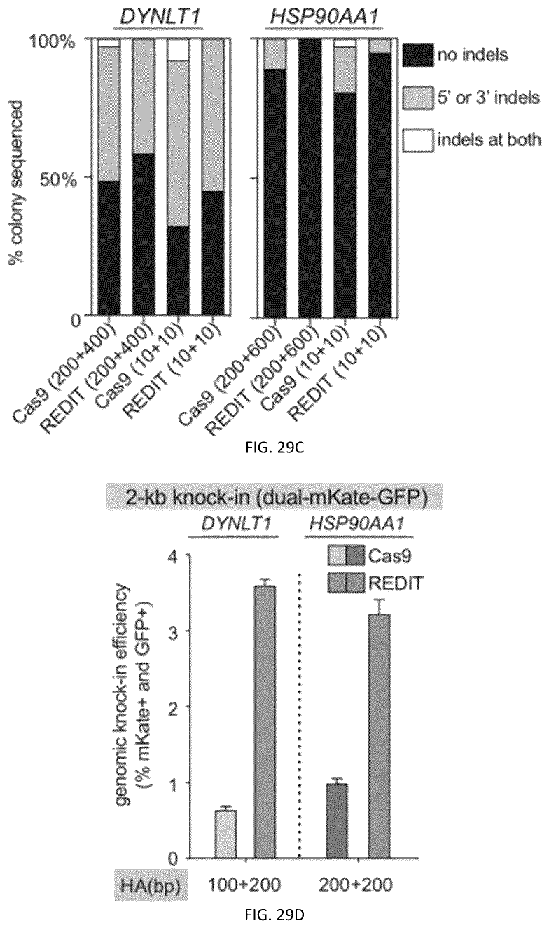

The effect of template HA lengths on the editing efficiency of REDIT was quantified when using the canonical HDR donor bearing HAs of at least 100 bp on each side (FIG. 29A, left). Higher HDR rates were observed for both Cas9 and RecT groups with increasing HA lengths, and REDIT effectively stimulated HDR over Cas9 using HA lengths as short as ˜100 bp each side. When supplied with a longer template bearing 600-800 bp total HA, RecT achieved over 10% HDR efficiencies for kb-scale knock-in without selection, significantly higher than the 2-3% efficiency when only using Cas9. Recent reports identified that using donor DNAs with shorter HAs (usually between 10 and 50 bp) could significantly stimulate knock-in efficiencies thanks to the high repair activities from the Microhomology-mediated end joining (MMEJ) pathway. Knock-in efficiencies of the REDIT-based method were compared with Cas9, using donor DNA with Obp (NHEJ-based), 10 bp or 50 bp (MMEJ-based) HAs. The results demonstrated that short-HA donors leveraging MMEJ mechanisms yielded higher editing efficiencies compared with HDR donors (FIG. 29A, right). At the same time, REDIT was able to enhance the knock-in efficiencies as long as there is HA present (no effect for the Obp NHEJ donor). This effect is particularly significant with The 10 bp donors in which there was a significant effect, were chosen for further characterization and comparison with the HDR donors.

The knock-in cells were clonally isolated and the target genomic region was amplified using primers binding completely outside of the donor DNAs for colony Sanger sequencing (FIG. 29B. Junction sequencing analysis (˜48 colonies per gene per condition) revealed varying degrees of indels at 5′- and 3′-knock-in junctions, including at single or both junctions (FIG. 29C). Overall, HDR donors had better precision than MMEJ donors, and REDIT modestly improved the knock-in yield compared with Cas9, though junction indels were still observed.

Furthermore, the efficiencies of REDIT and Cas9 were compared when making different lengths of editing. For longer edits, 2-kb knock-in cassettes were used (FIG. 29D), and for shorter edits single-stranded oligo donors (ssODN) were used. When the knock-in sequence length was increased to ˜2-kb using a dual-mKate/GFP template, REDIT maintained its HDR-promoting activity compared with Cas9 across endogenous targets tested (FIG. 29D). For ssODN tests, at two well-established loci EMX1 and VEGFA, REDIT and Cas9 were used to introduce 12-16-bp exogenous sequences. As ssODN templates are short (<100 bp HAs on each side), next-generation sequencing (NGS) was used to quantify the editing events. Comparable levels of indels were observed between Cas9 and REDIT with improved HDR efficiencies using REDIT.

### Example 14

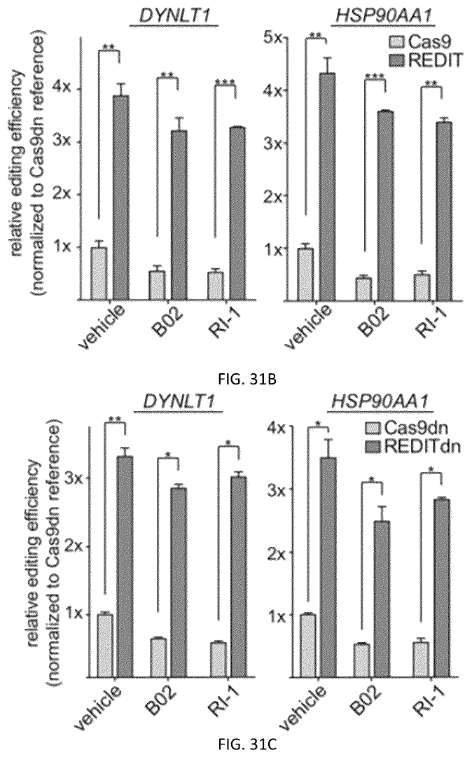

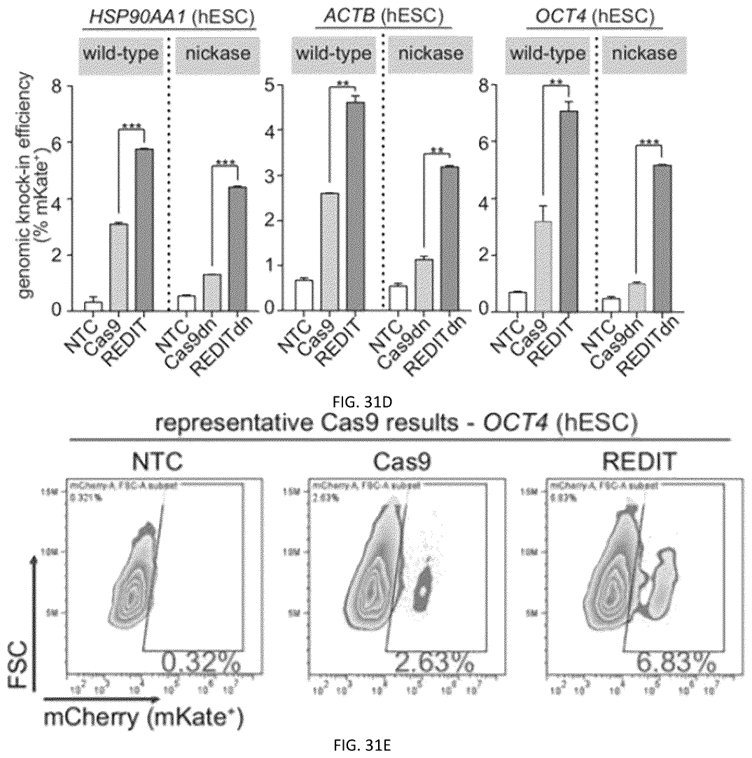

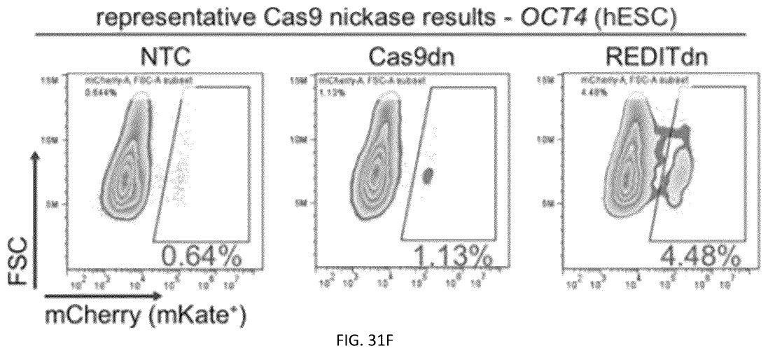

The sensitivity of REDIT's ability to promote HDR in the presence or absence of two distinctive pharmacological inhibitors of RAD51, B02 and RI-1 (FIG. 31A). As expected, for Cas9-based editing, RAD51 inhibition significantly lowered HDR efficiencies (FIGS. 31B, 31C, and 32A). Intriguingly, RAD51 inhibition decreased REDIT and REDITdn efficiencies only moderately, as both REDIT/REDITdn methods maintained significantly higher knock-in efficiencies compared with Cas9/Cas9dn under RAD51 inhibition.

Mirin, a potent chemical inhibitor of DSB repair, which has also been shown to prevent MRN complex formation, MRN-dependent ATM activation, and inhibit Mre11 exonuclease activity was also used. When treating cells with Mrining, only the editing efficiencies of Cas9 reference experiments were affected by the Miring treatment, whereas the REDIT versions were essentially the same as vehicle-treated groups across all genomic targets (FIG. 32A).

To test if cell cycle inhibition affected recombination, cells were chemically synchronized at the G1/S boundary using double Thymidine blockage (DTB). REDIT versions had reduced editing efficiencies under DTB treatment, though it maintained higher editing efficiencies under DNA repair pathway inhibition, compared with Cas9 reference experiments, when Miring RI-1, or B02 were combined with DTB treatment (FIG. 32B).

To validate REDIT in different contexts, REDIT was applied in human embryonic stem cells (hESCs) to test their ability to engineer long sequences in non-transformed human cells. Robust stimulation of HDR was observed across all three genomic sites (HSP90AA1, ACTB, OCT4/POU5F1) using REDIT and REDITdn (FIGS. 31D and 31E). Of note, REDIT and REDITdn editing used donor DNAs with 200-bp HAs on each side and achieved up to over 5% efficiency for kb-scale gene-editing without selection compared with ˜1% efficiency using non-REDIT methods. Additionally, REDIT improved knock-in efficiencies in A549 (lung-derived), HepG2 (liver-derived), and HeLa (cervix-derived) cells, demonstrating up to ˜15% kb-scale genomic knock-in without selection. This improvement was up to 4-fold higher than the Cas9 groups, supporting the potential of using REDIT methods in different cell types.

### Example 15

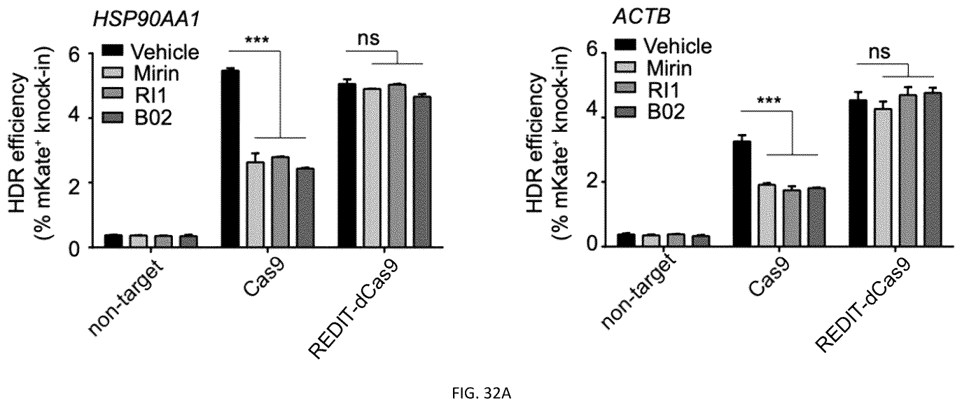

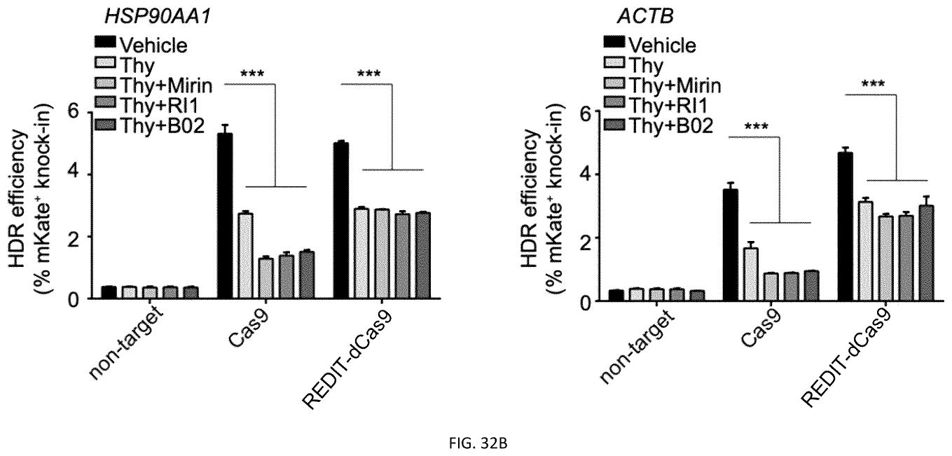

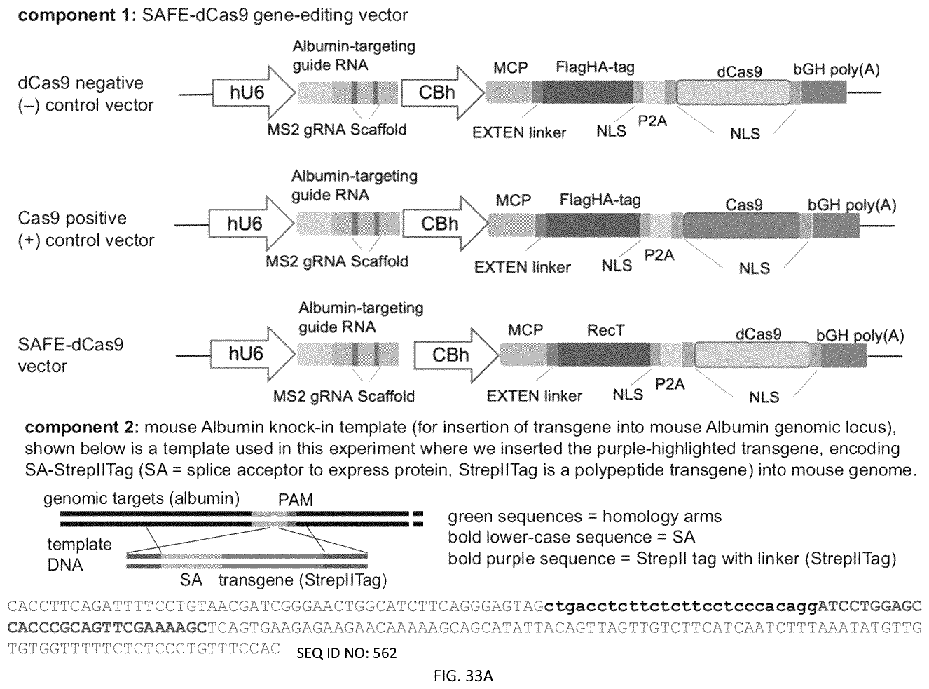

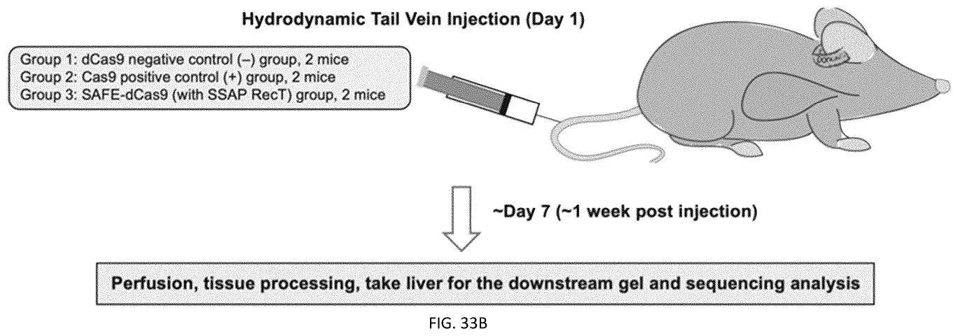

In vivo use of dCas9-EcRecT (SAFE-dCas9) was tested using cleavage free dCas9 editor via hydrodynamic tail vein injection. The gene editing vectors and template DNA used are shown in FIG. 33A. A gene editing vector (60 μg) and template DNA (60 μg) were injected via hydrodynamic tail vein injection to deliver the components to the mouse. Successful gene editing of liver hepatocytes was monitored by transgene-encoded protein expression from the albumin locus. A schematic of the experimental procedure is shown in FIG. 33B

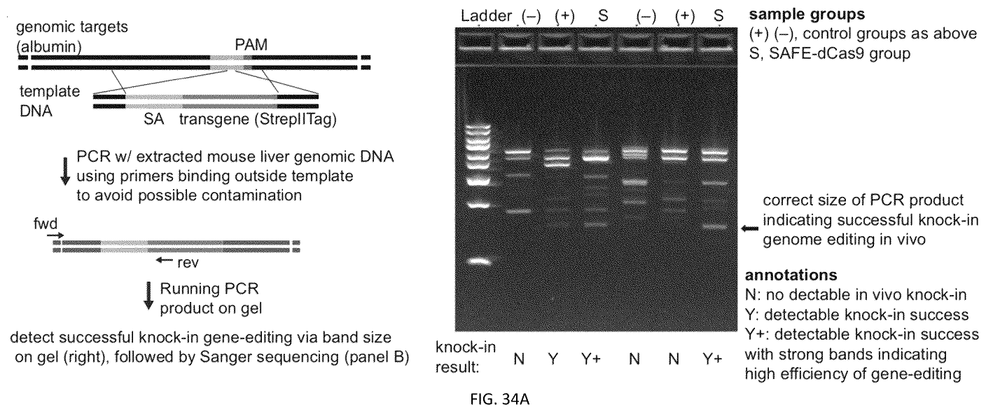

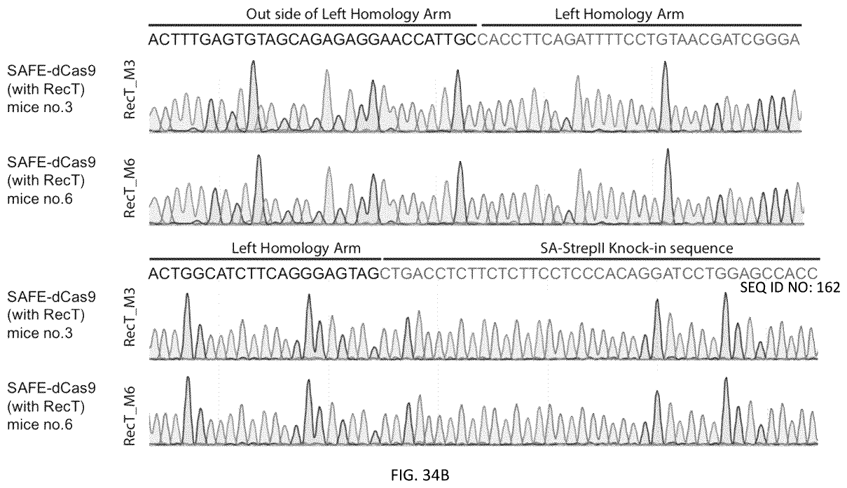

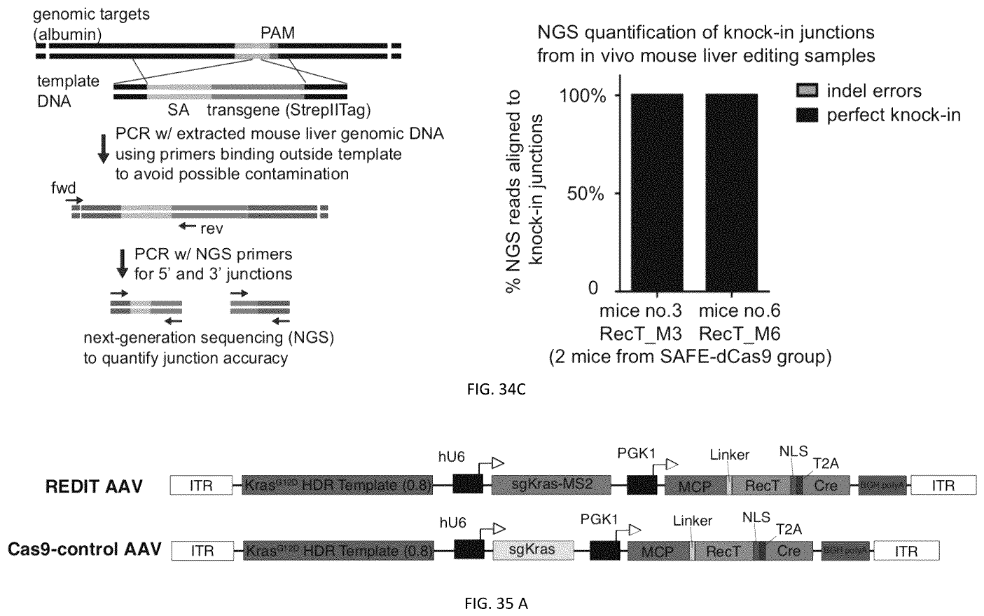

At approximately seven days after injection, the perfused mice livers were dissected. The lobes of the liver were homogenized and processed to extract liver genomic DNA from the primary hepatocytes. The extracted genomic DNA was used for three different downstream analyses: 1) PCR using knock-in-specific primers and agarose gel electrophoresis (FIG. 34A); 2) Sanger sequencing of the knock-in PCR product (FIG. 34B); 3) high-throughput deep sequencing of the knock-in junction to confirm and quantify the accuracy of gene-editing using SAFE-dCas9 in vivo (FIG. 34C). Each downstream analysis confirmed knock-in success with.

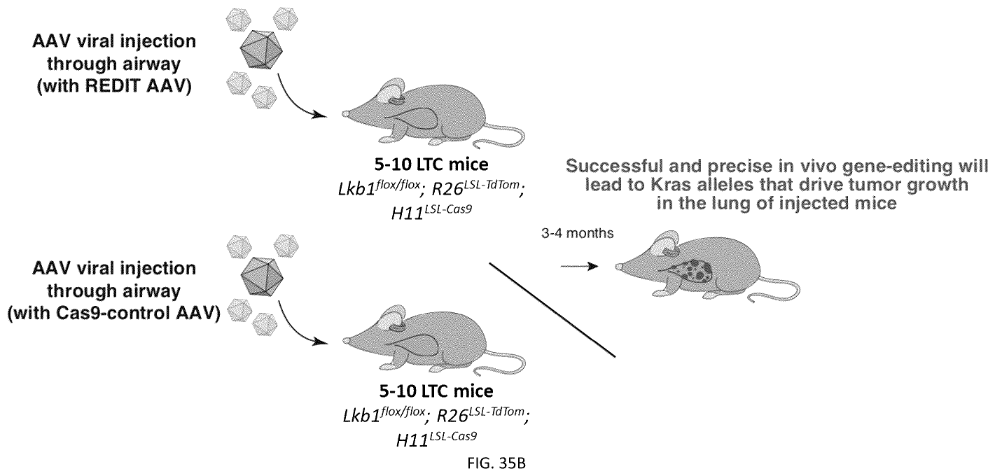

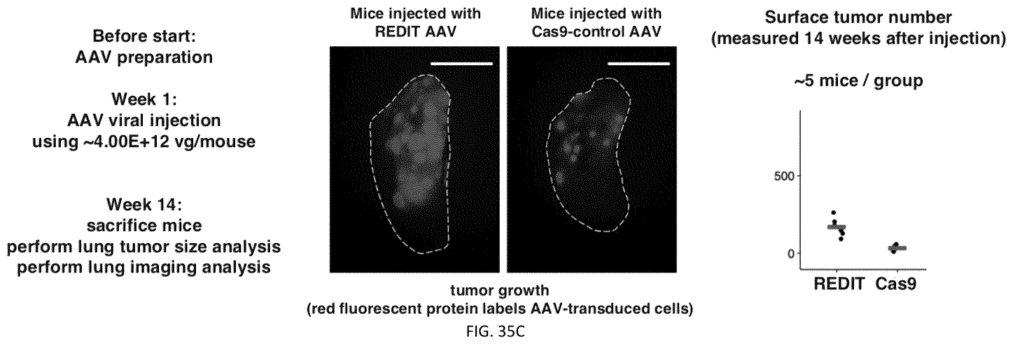

In addition, in vivo use was tested using adeno-associated virus (AAV) delivery into LTC mice lungs. LTC mice include three genome alleles: 1) Lkb1 (flox/flox) allele allows Lkb1-KO when expressing Cre; 2) R26 (LSL-TdTom) allele allows detection of AAV-transduced cells via TdTom red fluorescent protein; and 3) H11 (LSL-Cas9) allele allows expression of Cas9 in AAV-transduced cells. Schematics of the REDI gene editing vector and Cas9 control vectors are shown in FIG. 35A. As shown in FIG. 35B, successful gene editing using the gene editing vector leads to Kras alleles that drive tumor growth in the lung of the treated mice.

Approximately fourteen weeks after the AAV injection, perfused mice lungs were dissected. Fixed lung tissue was used for imaging analysis to identify tumor formation from successful gene-editing (FIG. 35C). Quantification of the surface tumor number via imagining analysis showed increased gene-editing efficiencies and total number of tumors in the REDIT treated mice (FIG. 35C).

Escherichia coli RecE amino acid sequence
(SEQ ID NO: 1):
MSTKPLFLLRKAKKSSGEPDVVLWASNDFESTCATLDYLIVKSGK
KLSSYFKAVATNFPVVNDLPAEGEIDFTWSERYQLSKDSMTWELK
PGAAPDNAHYQGNTNVNGEDMTEIEENMLLPISGQELPIRWLAQH
GSEKPVTHVSRDGLQALHIARAEELPAVTALAVSHKTSLLDPLEI
RELHKLVRDTDKVFPNPGNSNLGLITAFFEAYLNADYTDRGLLTK
EWMKGNRVSHITRTASGANAGGGNLTDRGEGFVHDLTSLARDVAT
GVLARSMDLDIYNLHPAHAKRIEEIIAENKPPFSVFRDKFITMPG
GLDYSRAIVVASVKEAPIGIEVIPAHVTEYLNKVLTETDHANPDP
EIVDIACGRSSAPMPQRVTEEGKQDDEEKPQPSGTTAVEQGEAET
MEPDATEHHQDTQPLDAQSQVNSVDAKYQELRAELHEARKNIPSK
NPVDDDKLLAASRGEFVDGISDPNDPKWVKGIQTRDCVYQNQPET
EKTSPDMNQPEPVVQQEPEIACNACGQTGGDNCPDCGAVMGDATY
QETFDEESQVEAKENDPEEMEGAEHPHNENAGSDPHRDCSDETGE
VADPVIVEDIEPGIYYGISNENYHAGPGISKSQLDDIADTPALYL
WRKNAPVDTTKTKTLDLGTAFHCRVLEPEEFSNRFIVAPEFNRRT
NAGKEEEKAFLMECASTGKTVITAEEGRKIELMYQSVMALPLGQW
LVESAGHAESSIYWEDPETGILCRCRPDKIIPEFHWIMDVKTTAD
IQRFKTAYYDYRYHVQDAFYSDGYEAQFGVQPTFVFLVASTTIEC
GRYPVEIFMMGEEAKLAGQQEYHRNLRTLADCLNTDEWPAIKTLS
LPRWAKEYAND
Escherichia coli RecE_587 amino acid sequence
(SEQ ID NO: 2):
ADPVIVEDIEPGIYYGISNENYHAGPGVSKSQLDDIADTPALYLW
RKNAPVDTTKTKTLDLGTAFHCRVLEPEEFSNRFIVAPEFNRRTN
SGKEEEKAFLRECASTGKTVITAEEGRKIELMYQSVMALPLGQWL
VESAGHAESSIYWEDPETAILCRCRPDKIIPEFHWIMDVKTTADI
QRFKTAYYDYRYHVQDAFYSDGYEAQFGVQPTFVFLVASTTIECG
RYPVEIFMMGEEAKLAGQLEYHRNLRTLADCLNTDEWPAIKTLSL
PRWAKEYAND*
Escherichia coli CTD_RecE amino acid sequence
(SEQ ID NO: 3):
GISNENYHAGPGVSKSQLDDIADTPALYLWRKNAPVDTTKTKTLD
LGTAFHCRVLEPEEFSNRFIVAPEFNRRTNSGKEEEKAFLRECAS
TGKTVITAEEGRKIELMYQSVMALPLGQWLVESAGHAESSIYWED
PETAILCRCRPDKIIPEFHWIMDVKTTADIQRFKTAYYDYRYHVQ
DAFYSDGYEAQFGVQPTFVFLVASTTIECGRYPVEIFMMGEEAKL
AGQLEYHRNLRTLADCLNTDEWPAIKTLSLPRWAKEYAND*
Pantoea brenneri RecE amino acid sequence
(SEQ ID NO: 4):
MQPGIYYDISNEDYHRGAGISKSQLDDIAISPAIYQWRKHAPVDE
EKTAALDLGTALHCLLLEPDEFSKRFQIGPEVNRRTTAGKEKEKE
FIERCEAEGITPITHDDNRKLKLMRDSALAHPIARWMLEAQGNAE
ASIYWNDRDAGVLSRCRPDKIITEFNWCVDVKSTADIMKFQKDFY
SYRYHVQDAFYSDGYESHFHETPTFAFLAVSTSIDCGRYPVQVFI
MDQQAKDAGRAEYKRNIHTFAECLSRNEWPGIATLSLPFWAKELR
NE
Type-F symbiont of Plautia stali RecE amino
acid sequence
(SEQ ID NO: 5):
MQPGIYYDISNEDYHGGPGISKSQLDDIAISPAIYQWRKHAPVDE
EKTAALDLGTALHCLLLEPDEFSKRFEIGPEVNRRTTAGKEKEKE
FMERCEAEGVTPITHDDNRKLRLMRDSAMAHPIARWMLEAQGNAE
ASIYWNDRDTGVLSRCRPDKIITDFNWCVDVKSTADIIKFQKDFY
SYRYHVQDAFYSDGYESHFDETPTFAFLAVSTSIDCGRYPVQVFI
MDQQAKDAGRAEYKRNIHTFAECLSRNEWPGIATLSLPYWAKELR
NE
Providencia sp. MGF014 RecE amino acid
sequence
(SEQ ID NO: 6):
MKEGIYYNISNEDYHNGLGISKSQLDLINEMPAEYIWSKEAPVDE
EKIKPLEIGTALHCLLLEPDEYHKRYKIGPDVNRRTNVGKEKEKE
FFDMCEKEGITPITHDDNRKLMIMRDSALAHPIAKWCLEADGVSE
SSIYWTDKETDVLCRCRPDRIITAHNYIIDVKSSGDIEKFDYEYY
NYRYHVQDAFYSDGYKEVTGITPTFLFLVVSTKIDCGKYPVRTYV
MSEEAKSAGRTAYKHNLLTYAECLKTDEWAGIRTLSLPRWAKELR
NE
Shigella sonnei RecE amino acid sequence
(SEQ ID NO: 7):
DRGLLTKEWRKGNRVSRITRTASGANAGGGNLTDRGEGFVHDLTS
LARDIATGVLARSMDVDIYNLHPAHAKRIEEIIAENKPPFSVFRD
KFITMPGGLDYSRAIVVASVKEAPIGIEVIPAHVTAYLNKVLTET
DHANPDPEIVDIACGRSSAPMPQRVTEEGKQDDEEKLQPSGTTAD
EQGEAETMEPDATKHHQDTQPLDAQSQVNSVDAKYQELRAELHEA
RKNIPSKNPVDADKLLAASRGEFVDGISDPNDPKWVKGIQTRDSV
YQNQPETEKTSPDMKQPEPVVQQEPEIAFNACGQTGGDNCPDCGA
VMGDATYQETFDEENQVEAKENDPEEMEGAEHPHNENAGSDPHRD
CSDETGEVADPVIVEDIEPGIYYGISNENYHAGPGVSKSQLDDIA
DTPALYLWRKNAPVDTTKTKTLDLGTAFHCRVLEPEEFSNRFIVA
PEFNRRTNAGKEEEKAFLMECASTGKMVITAEEGRKIELMYQSVM
ALPLGQWLVESAGHAESSIYWEDPETGILCRCRPDKIIPEFHWIM
DVKTTADIQRFKTAYYDYRYHVQDAFYSDGYEAQFGVQPTFVFLV
ASTTIECGRYPVEIFMMGEEAKLAGQLEYHRNLRTLADCLNTDEW
PAIKTLSLPRWAKEYAND
Pseudobacteriovorax antillogorgiicola RecE
amino acid sequence
(SEQ ID NO: 8):
MSKLSNLKVSNSDVDTLSRIRMKEGVYRDLPIESYHQSPGYSKTS
LCQIDKAPIYLKTKVPQKSTKSLNIGTAFHEAMEGVFKDKYVVHP
DPGVNKTTKSWKDFVKRYPKHMPLKRSEYDQVLAMYDAARSYRPF
QKYHLSRGFYESSFYWHDAVTNSLIKCRPDYITPDGMSVIDFKTT
VDPSPKGFQYQAYKYHYYVSAALTLEGIEAVTGIRPKEYLFLAVS
NSAPYLTALYRASEKEIALGDHFIRRSLLTLKTCLESGKWPGLQE
EILELGLPFSGLKELREEQEVEDEFMELVG
Escherichia coli RecT amino acid sequence
(SEQ ID NO: 9):
MTKQPPIAKADLQKTQGNRAPAAVKNSDVISFINQPSMKEQLAAA
LPRHMTAERMIRIATTEIRKVPALGNCDTMSFVSAIVQCSQLGLE
PGSALGHAYLLPFGNKNEKSGKKNVQLIIGYRGMIDLARRSGQIA
SLSARVVREGDEFSFEFGLDEKLIHRPGENEDAPVTHVYAVARLK
DGGTQFEVMTRKQIELVRSLSKAGNNGPWVTHWEEMAKKTAIRRL
FKYLPVSIEIQRAVSMDEKEPLTIDPADSSVLTGEYSVIDNSEE*
Pantoea brenneri RecT amino acid sequence
(SEQ ID NO: 10):
MSNQPPIASADLQKTQQSKQVANKTPEQTLVGFMNQPAMKSQLAA
ALPRHMTADRMIRIVTTEIRKTPQLAQCDQSSFIGAVVQCSQLGL
EPGSALGHAYLLPFGNGRSKSGQSNVQLIIGYRGMIDLARRSGQI
VSLSARVVRADDEFSFEYGLDENLVHRPGENEDAPITHVYAVARL
KDGGTQFEVMTVKQVEKVKAQSKASSNGPWVTHWEEMAKKTVIRR
LFKYLPVSIEMQKAVVLDEKAESDVDQDNASVLSAEYSVLESGDE
ATN
Type-F symbiont of Plautia stali RecT amino
acid sequence
(SEQ ID NO: 11):
MSNQPPIASADLQKTQQSKQVANKTPEQTLVGFMNQPAMKSQLAA
ALPRHMTADRMIRIVTTEIRKTPALATCDQSSFIGAVVQCSQLGL
EPGSALGHAYLLPFGNGRSKSGQSNVQLIIGYRGMIDLARRSGQI
VSLSARVVRADDEFSFEYGLDENLIHRPGDNEDAPITHVYAVARL
KDGGTQFEVMTAKQVEKVKAQSKASSNGPWVTHWEEMAKKTVIRR
LFKYLPVSIEMQKAVVLDEKAESDVDQDNASVLSAEYSVLEGDGG
E
Providencia sp. MGF014 RecT amino acid
sequence
(SEQ ID NO: 12):
MSNPPLAQSDLQKTQGTEVKVKTKDQQLIQFINQPSMKAQLAAAL
PRHMTPDRMIRIVTTEIRKTPALATCDMQSFVGAVVQCSQLGLEP
GNALGHAYLLPFGNGKAKSGQSNVQLIIGYRGMIDLARRSNQIIS
ISARTVRQGDNFHFEYGLNEDLTHTPSENEDSPITHVYAVARLKD
GGVQFEVMTYNQVEKVRASSKAGQNGPWVSHWEEMAKKTVIRRLF
KYLPVSIEMQKAVVLDEKAEANVDQENATIFEGEYEEVGTDGN
Shigella sonnei RecT amino acid sequence
(SEQ ID NO: 13):
MTKQPPIAKADLQKTQENRAPAAIKNNDVISFINQPSMKEQLAAA
LPRHMTAERMIRIATTEIRKVPALGNCDTMSFVSAIVQCSQLGLE
PGSALGHAYLLPFGNKNEKSGKKNVQLIIGYRGMIDLARRSGQIA
SLSARVVREGDEFNFEFGLDEKLIHRPGENEDAPVTHVYAVARLK
DGGTQFEVMTRRQIELVRSQSKAGNNGPWVTHWEEMAKKTAIRRL
FKYLPVSIEIQRAVSMDEKEPLTIDPADSSVLTGEYSVIDNSEE
Pseudobacteriovorax antillogorgiicola RecT
amino acid sequence
(SEQ ID NO: 14):
MGHLVSKTEQDYIKQHYAKGATDQEFEHFIGVCRARGLNPAANQI
YFVKYRSKDGPAKPAFILSIDSLRLIAHRTGDYAGCSEPIFTDGG
KACTVTVRRNLKSGETGNFSGMAFYDEQVQQKNGRPTSFWQSKPR
TMLEKCAEAKALRKAFPQDLGQFYIREEMPPQYDEPIQVHKPKAL
EEPRESKSDLSRRKGLNRKLSALGVDPSRFDEVATFLDGTPDREL
GQKLKLWLKEAGYGVNQ
SV40 NLS amino acid sequence
(SEQ ID NO: 16):
PKKKRKV
Ty1 NLS amino acid sequence
(SEQ ID NO: 17):
NSKKRSLEDNETEIKVSRDTWNTKNMRSLEPPRSKKRIH
c-Myc NLS amino acid sequence
(SEQ ID NO: 18):
PAAKRVKLD
biSV40 NLS amino acid sequence
(SEQ ID NO: 19):
KRTADGSEFESPKKKRKV
Mut NLS amino acid sequence
(SEQ ID NO: 20):
PEKKRRRPSGSVPVLARPSPPKAGKSSCI
Template DNA sequences (underlining marks
the replaced or inserter editing sequences)
EMX1 HDR template sequence
(SEQ ID NO: 79):
CATTCTGCCTCTCTGTATGGAAAAGAGCATGGGGCTGGCCCGTGG
GGTGGTGTCCACTTTAGGCCCTGTGGGAGATCATGGGAACCCACG
CAGTGGGTcataggctctctcatttactactcacatccactctgt
gaagaagcgattatgatctctcctctagaaaCTCGTAGAGTCCCA
TGTCTGCCGGCTTCCAGAGCCTGCACTCCTCCACCTTGGCTTGGC
TTTGCTGGGGCTAGAGGAGCTAGGATGCACAGCAGCTCTGTGACC
CTTTGTTTGAGAGGAACAGGAAAACCACCCTTCTCTCTGGCCCAC
TGTGTCCTCTTCCTGCCCTGCCATCCCCTTCTGTGAATGTTAGAC
CCATGGGAGCAGCTGGTCAGAGGGGACCCCGGCCTGGGGCCCCTA
ACCCTATGTAGCCTCAGTCTTCCCATCAGGCTCTCAGCTCAGCCT
GAGTGTTGAGGCCCCAGTGGCTGCTCTGGGGGCCTCCTGAGTTTC
TCATCTGTGCCCCTCCCTCCCTGGCCCAGGTGAAGGTGTGGTTCC
AGAACCGGAGGACAAAGTACAAACGGCAGAAGCTGGAGGAGGAAG
GGCCTGAGTCCGAGCAGAAGAAGAAGGGCTCCCATCACATCAACC
GGTGGCGCATTGCCACGAAGCAGGCCAATGGGGAGGACATCGATG
TCACCTCCAATGACTCGGATGTACACGGTCTGCAACCACAAACCC
ACGAGGGCAGAGTGCTGCTTGCTGCTGGCCAGGCCCCTGCGTGGG
CCCAAGCTGGACTCTGGCCACTCCCTGGCCAGGCTTTGGGGAGGC
CTGGAGTCATGGCCCCACAGGGCTTGAAGCCCGGGGCCGCCATTG
ACAGAGGGACAAGCAATGGGCTGGCTGAGGCCTGGGACCACTTGG
CCTTCTCCTCGGAGAGCCTGCCTGCCTGGGCGGGCCCGCCCGCCA
CCGCAGCCTCCCAGCTGCTCTCCGTGTCTCCAATCTCCCTTTTGT
TTTGATGCATTTCTGTTTTAATTTATTTTCCAGGCACCACTGTAG
TTTAGTGATCCCCAGTGTCCCCCTTCCCTATGGGAATAATAAAAG
TCTCTCTCTTAATGACACGGGCATCCAGCTCCAGCCCCAGAGCCT
GGGGTGGTAGATTCCGGCTCTGAGGGCCAGTGGGGGCTGGTAGAG
CAAACGCGTTCAGGGCCTGGGAGCCTGGGGTGGGGTACTGGTGGA
GGGGGTCAAGGGTAATTCATTAACTCCTCTCTTTTGTTGGGGGAC
CCTGGTCTCTACCTCCAGCTCCACAGCAGGAGAAACAGGCTAGAC
ATAGGGAAGGGCCATCCTGTATCTTGAGGGAGGACAGGCCCAGGT
CTTTCTTAACGTATTGAGAGGTGGGAATCAGGCCCAGGTAGTTCA
ATGGG
VEGFA HDR template sequence
(SEQ ID NO: 80):
AGGTTTGAATCATCACGCAGGCCCTGGCCTCCACCCGCCCCCACC
AGCCCCCTGGCCTCAGTTCCCTGGCAACATCTGGGGTTGGGGGGG
CAGCAGGAACAAGGGCCTCTGTCTGCCCAGCTGCCTCCCCCTTTG
GGTTTTGCCAGACTCCACAGTGCATACGTGGGCTCCAACAGGTCC
TCTTCCCTCCCAGTCACTGACTAACCCCGGAACCACACAGCTTCC
CGTTctcagctccacaaacttggtgccaaattcttctcccctggg
aagcatccctggacacttcccaaaggaccccagtcactccagcct
gttggctgccgctcactttgatgtctgcaggccagatgagggctc
cagatggcacattgtcagagggacacactgtggcccctgtgccca
gccctgggctctctgtacatgaagcaactccagtcccaaatatgt
agctgtttgggaggtcagaaatagggggtccaggagcaaactccc
cccaccccctttccaaagcccattccctctttagccagagccggg
gtgtgcagacggcagtcactagggggcgctcggccaccacaggga
agctgggtgaatggagcgagcagcgtcttcgagagtgaggacgtg
tgtgtctgtgtgggtgagtgagtgtgCgcACTCTAGAGgtgtCgT
gttgagggcgttggagcggggagaaggccaggggtcactccagga
ttccaatagatctgtgtgtccctctccccacccgtccctgtccgg
ctctccgccttcccctgcccccttcaatattcctagcaaagaggg
aacggctctcaggccctgtccgcacgtaacctcactttcctgctc
cctcctcgccaatgccccgcgggcgcgtgtctctggacagagttt
ccgggggcggatgggtaattttcaggctgtgaaccttggtggggg
tcgagcttccccttcattgcgggggctGCGGGCCAGGCTTCACTG
AGCGTCCGCAGAGCCCGGGCCCGAGCCGCGTGTGGAAGGGCTGAG
GCTCGCCTGTccccgccccccggggcgggccgggggggggtcccg
gcggggcggAGCCATGCGCCCCCCCCttttttttttAAAAGTCGG
CTGGTAGCGGGGAGGATCGCGGAGGCTTGGGGCAGCCGGGTAGCT
CGGAGGTCGTGGCGCTGGGGGCTAGCACCAGCGCTCTGTCGGGAG
GCGCAGCGGTTAGGTGGACCGGTCAGCGGACTCACCGGCCAGGGC
GCTCGGTGCTGGAATTTGATATTCATTGATCCGGGttttatccct
cttcttttttcttaaacatttttttttAAAACTGTATTGTTTCTC
GTTTTAATTTATTTTTGCTTGCCATTCCCCACTTGAAT
DYNLT1 HDR template sequence
(SEQ ID NO: 81):
AGTGACCTGTGTAATTATGCAGAAGAATGGAGCTGGATTACACAC
AGCAAGTTCCTGCTTCTGGGACAGCTCTACTGACGGTATGATTTT
CATTCATGTTTGTGAAGTTTTGTTGTGTGAAATATATGACTGGAA
GTTTCCTATCTTTGAATGCAATGCATGTTTATCACCTTTTAAAAC
ATTTAATAATAGACTTGCCAAGGTTCTTTGTGTAGCATAGAGATG
GGTACTTGAATGTTGGCCTTATTGTGAGTAAAACGTCGTCCCCCA
GCTTTCCCTGCCGTAAATGCTGCTCTCTTCCCTCCCGCAGGGAGC
TGCACTGTGCGATGGGAGAATAAGACCATGTACTGCATCGTCAGT
GCCTTCGGACTGTCTATTGGAAGCGGAGCTACTAACTTCAGCCTG
CTGAAGCAGGCTGGAGACGTGGAGGAGAACCCTGGACCTgccacc
atggtgagcgagctgattaaggagaacatgcacatgaagctgtac
atggagggcaccgtgaacaaccaccacttcaagtgcacatccgag
ggcgaaggcaagccctacgagggcacccagaccatgagaatcaag
gcggtcgagggcggccctctccccttcgccttcgacatcctggct
accagcttcatgtacggcagcaaaaccttcatcaaccacacccag
ggcatccccgacttctttaagcagtccttccccgagggcttcaca
tgggagagagtcaccacatacgaagatgggggcgtgctgaccgct
acccaggacaccagcctccaggacggctgcctcatctacaacgtc
aagatcagaggggtgaacttcccatccaacggccctgtgatgcag
aagaaaacactcggctgggaggcctccaccgagacactgtacccc
gctgacggcggcctggaaggcagagccgacatggccctgaagctc
gtggggggggccacctgatctgcaaccttaagaccacatacagat
ccaagaaacccgctaagaacctcaagatgcccggcgtctactatg
tggacaggagactggaaagaatcaaggaggccgacaaagagacat
acgtcgagcagcacgaggtggctgtggccagatactgcgacctcc
ctagcaaactggggcacaaacttaattccTAACCaGCtGTCCtGC
CTATGGCCTTTCTCCTTTTGTCTCTAGTTCATCCTCTAACCACCA
GCCATGAATTCAGTGAACTCTTTTCTCATTCTCTTTGTTTTGTGG
CACTTTCACAATGTAGAGGAAAAAACCAAATGACCGCACTGTGAT
GTGAATGGCACCGAAGTCAGATGAGTATCCCTGTAGGTCACCTGC
AGCCTGCGTTGCCACTTGTCTTAACTCTGAATATTTCATTTCAAA
GGTGCTAAAATCTGAAATCTGCTAGTGTGAAACTTGCTCTACTCT
CTGAAATGATTCAAATACACTAATTTTCCATACTTTATACTTTTG
TTAGAATAAATTATTCAAATCTAAAGTCTGTTGTGTTCTTCATAG
TCTGCATAGTATCATAAACG
HSP90AA1 HDR template sequence
(SEQ ID NO: 82):
GCAGCAAAGAAACACCTGGAGATAAACCCTGACCATTCCATTATT
GAGACCTTAAGGCAAAAGGCAGAGGCTGATAAGAACGACAAGTCT
GTGAAGGATCTGGTCATCTTGCTTTATGAAACTGCGCTCCTGTCT
TCTGGCTTCAGTCTGGAAGATCCCCAGACACATGCTAACAGGATC
TACAGGATGATCAAACTTGGTCTGGGTAAGCCTTATACTATGTAA
TGTTAAAAAGAAAATAAACACACGTGACATTGAAGAAAATGGTGA
ACTTTCAGTTATCCAAACTTGGAGCACCTTGTCCTGCTTGCTGCT
TGGAGGTATTAAAGTATGttttttttAGGGATAAGTAAGGTCTTA
CAAGAGCAAAGAAATGAAATTGAGACTCATATGTCCTGTAATACT
GTCTTGAAAGCAGATAGAAACCAAGAGTATTACCCTAATAGCTGG
CTTTAAGAAATCTTTGTAATATGAGGATTTTATTTTGGAAACAGG
TATTGATGAAGATGACCCTACTGCTGATGATACCAGTGCTGCTGT
AACTGAAGAAATGCCACCCCTTGAAGGAGATGACGACACATCACG
CATGGAAGAAGTAGACGGAAGCGGAGCTACTAACTTCAGCCTGCT
GAAGCAGGCTGGAGACGTGGAGGAGAACCCTGGACCTgtgagcga
gctgattaaggagaacatgcacatgaagctgtacatggagggcac
cgtgaacaaccaccacttcaagtgcacatccgagggcgaaggcaa
gccctacgagggcacccagaccatgagaatcaaggcggtcgaggg
ggccctctccccttcgccttcgacatcctggctaccagcttcatg
tacggcagcaaaaccttcatcaaccacacccagggcatccccgac
ttctttaagcagtccttccccgagggcttcacatgggagagagtc
accacatacgaagatgggggcgtgctgaccgctacccaggacacc
agcctccaggacggctgcctcatctacaacgtcaagatcagaggg
gtgaacttcccatccaacggccctgtgatgcagaagaaaacactc
ggctgggaggcctccaccgagacactgtaccccgctgacggcggc
ctggaaggcagagccgacatggccctgaagctcgtgggcgggggc
cacctgatctgcaaccttaagaccacatacagatccaagaaaccc
gctaagaacctcaagatgcccggcgtctactatgtggacaggaga
ctggaaagaatcaaggaggccgacaaagagacatacgtcgagcag
cacgaggtggctgtggccagatactgcgacctccctagcaaactg
gggcacaaacttaattccTAaATCTgTGGCTGAGGGATGACTTAC
CTGTTCAGTACTCTACAATTCCTCTGATAATATATTTTCAAGGAT
GTTTTTCTTTATTTTTGTTAATATTAAAAAGTCTGTATGGCATGA
CAACTACTTTAAGGGGAAGATAAGATTTCTGTCTACTAAGTGATG
CTGTGATACCTTAGGCACTAAAGCAGAGCTAGTAATGCTTTTTGA
GTTTCATGTTGGTTTATTTTCACAGATTGGGGTAACGTGCACTGT
AAGACGTATGTAACATGATGTTAACTTTGTGGTCTAAAGTGTTTA
GCTGTCAAGCCGGATGCCTAAGTAGACCAAATCTTGTTATTGAAG
TGTTCTGAGCTGTATCTTGATGTTTAGAAAAGTATTCGTTACATC
TTGTAGGATCTACTTTTTGAACTTTTCATTCCCTGTAGTTGACAA
TTCTGCATGTACTAGTCCTCTAGAAATAGGTTAAACTGAAGCAAC
TTGATGGAAGGATCTCTCCACAGGGCTTGTTTTCCAAAGAAAAGT
ATTGTTTGGAGGAGCAAAGTTAAAAGCCTACCTAAGCATATCGTA
AAGCTGTTCAAAAATAACTCAGACCCAGTCTTGTGGA
AAVS1 HDR template sequence
(SEQ ID NO: 83):
gatgctctttccggagcacttccttctcggcgctgcaccacgtga
tgtcctctgagcggatcctccccgtgtctgggtcctctccgggca
tctctcctccctcacccaaccccatgccgtcttcactcgctgggt
tcccttttccttctccttctggggcctgtgccatctctcgtttct
taggatggccttctccgacggatgtctcccttgcgtcccgcctcc
ccttcttgtaggcctgcatcatcaccgtttttctggacaacccca
aagtaccccgtctccctggctttagccacctctccatcctcttgc
tttctttgcctggacaccccgttctcctgtggattcgggtcacct
ctcactcctttcatttgggcagctcccctaccccccttacctctc
tagtctgtgctagctcttccagccccctgtcatggcatcttccag
gggtccgagagctcagctagtcttcttcctccaacccgggcccct
atgtccacttcaggacagcatgtttgctgcctccagggatcctgt
gtccccgagctgggaccaccttatattcccagggccggttaatgt
ggctctggttctgggtacttttatctgtcccctccaccccacagt
ggggcaagcttctgacctcttctcttcctcccacagggcctcgag
agatctggcagcggaGGAAGCGGAGCTACTAACTTCAGCCTGCTG
AAGCAGGCTGGAGACGTGGAGGAGAACCCTGGACCTgtgagcgag
ctgattaaggagaacatgcacatgaagctgtacatggagggcacc
gtgaacaaccaccacttcaagtgcacatccgagggcgaaggcaag
ccctacgagggcacccagaccatgagaatcaaggcggtcgagggc
ggccctctccccttcgccttcgacatcctggctaccagcttcatg
tacggcagcaaaaccttcatcaaccacacccagggcatccccgac
ttctttaagcagtccttccccgagggcttcacatgggagagagtc
accacatacgaagatgggggcgtgctgaccgctacccaggacacc
agcctccaggacggctgcctcatctacaacgtcaagatcagaggg
gtgaacttcccatccaacggccctgtgatgcagaagaaaacactc
ggctgggaggcctccaccgagacactgtaccccgctgacggcggc
ctggaaggcagagccgacatggccctgaagctcgtgggcgggggc
cacctgatctgcaaccttaagaccacatacagatccaagaaaccc
gctaagaacctcaagatgcccggcgtctactatgtggacaggaga
ctggaaagaatcaaggaggccgacaaagagacatacgtcgagcag
cacgaggtggctgtggccagatactgcgacctccctagcaaactg
gggcacaaacttaattccTAaactaggacaggattggtgacaga
aaagccccatccttaggcctcctccttcctagtctcctgatattg
ggtctaacccccacctcctgttaggcagattccttatctggtgac
acacccccatttcctggagccatctctctccttgccagaacctct
aaggtttgcttacgatggagccagagaggatcctgggagggagag
cttggcaggggggggagggaagggggggatgcgtgacctgcccgg
ttctcagtggccaccctgcgctaccctctcccagaacctgagctg
ctctgacgcggctgtctggtgcgtttcactgatcctggtgctgca
gcttccttacacttcccaagaggagaagcagtttggaaaaacaaa
atcagaataagttggtcctgagttctaactttggctcttcacctt
tctagtccccaatttatattgttcctccgtgcgtcagttttacct
gtgagataaggccagtagccagccccgtcctggcagggctgtggt
gaggaggggggtgtccgtgtggaaaactccctttgtgagaatggt
gcgtcctaggtgttcaccaggtcgtggccgcctctactccctttc
tctttctccatccttctttccttaaagagtccccagtgctatctg
ggacatattcctccgcccagagcagggtcccgcttccctaaggcc
ctgctctgggcttctgggtttgagtccttggc
OCT4 HDR template sequence
(SEQ ID NO: 84):
GCGACTATGCACAACGAGAGGATTTTGAGGCTGCTGGGTCTCCTT
TCTCAGGGGGACCAGTGTCCTTTCCTCTGGCCCCAGGGCCCCATT
TTGGTACCCCAGGCTATGGGAGCCCTCACTTCACTGCACTGTACT
CCTCGGTCCCTTTCCCTGAGGGGGAAGCCTTTCCCCCTGTCTCCG
TCACCACTCTGGGCTCTCCCATGCATTCAAAtGGAAGCGGAGCTA
CTAACTTCAGCCTGCTGAAGCAGGCTGGAGACGTGGAGGAGAACC
CTGGACCTgccaccatggtgagcgagctgattaaggagaacatgc
acatgaagctgtacatggagggcaccgtgaacaaccaccacttca
agtgcacatccgagggcgaaggcaagccctacgagggcacccaga
ccatgagaatcaaggcggtcgagggcggccctctccccttcgcct
tcgacatcctggctaccagcttcatgtacggcagcaaaaccttca
tcaaccacacccagggcatccccgacttctttaagcagtccttcc
ccgagggcttcacatgggagagagtcaccacatacgaagatgggg
gcgtgctgaccgctacccaggacaccagcctccaggacggctgcc
tcatctacaacgtcaagatcagaggggtgaacttcccatccaac
ggccctgtgatgcagaagaaaacactcggctgggaggcctccac
cgagacactgtaccccgctgacggcggcctggaaggcagagccg
acatggccctgaagctcgtggggggggccacctgatctgcaacc
ttaagaccacatacagatccaagaaacccgctaagaacctcaag
atgcccggcgtctactatgtggacaggagactggaaagaatcaag
gaggccgacaaagagacatacgtcgagcagcacgaggtggctgtg
gccagatactgcgacctccctagcaaactggggcacaaacttaat
tccTAaTGACTAGGAATGGGGGACAGGGGGAGGGGAGGAGCTAGG
GAAAGAAAACCTGGAGTTTGTGCCAGGGTTTTTGGGATTAAGTTC
TTCATTCACTAAGGAAGGAATTGGGAACACAAAGGGTGGGGGCAG
GGGAGTTTGGGGCAACTGGTTGGAGGGAAGGTGAAGTTCAATGAT
GCTCTTGATTTTAATCCCACATCATGTATCACTTTTTTCTTAAAT
AAAGAAGCCTGGGACACAGTAGATAGACACACTT
Pantoea stewartii RecT DNA
(SEQ ID NO: 85):
AGCAACCAGCCCCCTATCGCCTCCGCCGATCTGCAGAAGGCCAAC
ACCGGCAAGCAGGTGGCCAATAAGACCCCTGAGCAGACACTGGTG
GGCTTCATGAATCAGCCAGCAATGAAGAGCCAGCTGGCCGCCGCC
CTGCCAAGGCACATGACAGCCGATCGGATGATCAGAATCGTGACC
ACAGAGATCCGCAAGACCCCCGCCCTGGCCACATGCGACCAGAGC
TCCTTCATCGGCGCCGTGGTGCAGTGTTCTCAGCTGGGCCTGGAG
CCTGGCAGCGCCCTGGGCCACGCCTACCTGCTGCCATTTGGCAAC
GGCCGGAGCAAGTCCGGACAGTCCAATGTGCAGCTGATCATCGGC
TATAGAGGCATGATCGATCTGGCCCGGAGATCTGGCCAGATCGTG
TCTCTGAGCGCCAGGGTGGTGCGCGCAGACGATGAGTTCTCCTTT
GAGTACGGCCTGGATGAGAACCTGATCCACCGGCCAGGCGAGAAT
GAGGACGCACCCATCACCCACGTGTATGCAGTGGCAAGACTGAAG
GACGGAGGCACCCAGTTCGAAGTGATGACAGTGAAGCAGATCGAG
AAGGTGAAGGCCCAGTCCAAGGCCTCTAGCAACGGACCCTGGGTG
ACCCACTGGGAGGAGATGGCCAAGAAAACCGTGATCAGGCGCCTG
TTTAAGTACCTGCCCGTGAGCATCGAGATGCAGAAGGCCGTGATC
CTGGATGAGAAGGCCGAGTCTGACGTGGATCAGGACAATGCCTCC
GTGCTGTCTGCCGAGTATAGCGTGCTGGACGGCTCCTCTGAGGAG
Pantoea stewartii RecE DNA
(SEQ ID NO: 86):
CAGCCCGGCGTGTACTATGACATCTCCAACGAGGAGTATCACGCC
GGCCCTGGCATCAGCAAGTCCCAGCTGGACGACATCGCCGTGTCC
CCAGCCATCTTCCAGTGGAGAAAGTCTGCCCCCGTGGACGATGAG
AAAACCGCCGCCCTGGACCTGGGCACAGCCCTGCACTGCCTGCTG
CTGGAGCCTGATGAGTTCTCCAAGAGGTTTATGATCGGCCCAGAG
GTGAACCGGAGAACCAATGCCGGCAAGCAGAAGGAGCAGGACTTC
CTGGATATGTGCGAGCAGCAGGGCATCACCCCTATCACACACGAC
GATAACCGGAAGCTGAGACTGATGAGGGACTCTGCCTTTGCCCAC
CCAGTGGCCAGATGGATGCTGGAGACAGAGGGCAAGGCCGAGGCC
TCTATCTACTGGAATGACAGGGATACACAGATCCTGAGCAGGTGC
CGCCCCGACAAGCTGATCACCGAGTTCTCTTGGTGCGTGGACGTG
AAGAGCACAGCCGACATCGGCAAGTTCCAGAAGGACTTCTACAGC
TATCGCTACCACGTGCAGGACGCCTTCTATTCCGATGGCTACGAG
GCCCAGTTTTGCGAGGTGCCAACCTTCGCCTTTCTGGTGGTGAGC
TCCTCTATCGATTGTGGCCGGTATCCCGTGCAGGTGTTTATCATG
GACCAGCAGGCAAAGGATGCAGGAAGGGCCGAGTATAAGCGGAAC
CTGACCACATACGCCGAGTGCCAGGCAAGGAATGAGTGGCCTGGC
ATCGCCACACTGAGCCTGCCTTACTGGGCCAAGGAGATCCGGAAT
GTG
Pantoea brenneri RecT DNA
(SEQ ID NO: 87):
AGCAACCAGCCCCCTATCGCCTCCGCCGATCTGCAGAAAACCCAG
CAGTCCAAGCAGGTGGCCAACAAGACCCCTGAGCAGACACTGGTG
GGCTTCATGAATCAGCCAGCAATGAAGAGCCAGCTGGCCGCCGCC
CTGCCAAGGCACATGACCGCCGATCGGATGATCAGAATCGTGACC
ACAGAGATCCGCAAGACACCACAGCTGGCCCAGTGCGACCAGAGC
TCCTTCATCGGCGCCGTGGTGCAGTGTTCTCAGCTGGGCCTGGAG
CCTGGCAGCGCCCTGGGCCACGCCTACCTGCTGCCATTTGGCAAC
GGCCGGTCCAAGTCTGGCCAGAGCAATGTGCAGCTGATCATCGGC
TATAGAGGCATGATCGATCTGGCCCGGAGATCCGGACAGATCGTG
AGCCTGTCCGCCAGGGTGGTGCGCGCAGACGATGAGTTCTCTTTT
GAGTACGGCCTGGATGAGAACCTGGTGCACCGGCCAGGCGAGAAT
GAGGACGCACCCATCACCCACGTGTATGCAGTGGCAAGACTGAAG
GACGGAGGCACCCAGTTCGAAGTGATGACAGTGAAGCAGGTGGAG
AAGGTGAAGGCCCAGTCCAAGGCCTCTAGCAATGGCCCCTGGGTG
ACCCACTGGGAGGAGATGGCCAAGAAAACCGTGATCAGGCGCCTG
TTTAAGTACCTGCCCGTGAGCATCGAGATGCAGAAGGCCGTGGTG
CTGGATGAGAAGGCCGAGTCTGACGTGGATCAGGACAACGCCTCT
GTGCTGAGCGCCGAGTATTCCGTGCTGGAGTCTGGCGACGAGGCC
ACAAAT
Pantoea brenneri RecE DNA
(SEQ ID NO: 88):
CAGCCTGGCATCTACTATGACATCAGCAACGAGGATTATCACAGG
GGAGCAGGCATCAGCAAGTCCCAGCTGGACGACATCGCCATCTCC
CCAGCCATCTACCAGTGGAGAAAGCACGCCCCCGTGGACGAGGAG
AAAACCGCCGCCCTGGATCTGGGCACAGCCCTGCACTGCCTGCTG
CTGGAGCCTGACGAGTTCTCTAAGAGGTTTCAGATCGGCCCAGAG
GTGAACCGGAGAACCACAGCCGGCAAGGAGAAGGAGAAGGAGTTC
ATCGAGCGGTGCGAGGCAGAGGGAATCACCCCAATCACACACGAC
GATAATAGGAAGCTGAAGCTGATGAGGGATTCCGCCCTGGCCCAC
CCAATCGCAAGGTGGATGCTGGAGGCACAGGGAAACGCAGAGGCC
TCTATCTATTGGAATGACAGAGATGCCGGCGTGCTGAGCAGGTGC
CGCCCCGACAAGATCATCACCGAGTTCAACTGGTGCGTGGACGTG
AAGTCCACAGCCGACATCATGAAGTTCCAGAAGGACTTCTACTCT
TACAGATACCACGTGCAGGACGCCTTCTATTCCGATGGCTACGAG
TCTCACTTTCACGAGACACCCACATTCGCCTTTCTGGCCGTGTCT
ACCAGCATCGACTGCGGCAGGTATCCTGTGCAGGTGTTTATCATG
GACCAGCAGGCAAAGGATGCAGGAAGGGCCGAGTACAAGAGAAAC
ATCCACACCTTCGCCGAGTGTCTGAGCAGGAATGAGTGGCCTGGC
ATCGCCACACTGTCCCTGCCTTTTTGGGCCAAGGAGCTGCGCAAT
GAG
Pantoea dispersa RecT DNA
(SEQ ID NO: 89):
TCCAACCAGCCACCTCTGGCCACCGCAGATCTGCAGAAAACCCAG
CAGTCTAACCAGGTGGCCAAGACCCCTGAGCAGACACTGGTGGGC
TTCATGAATCAGCCAGCAATGAAGAGCCAGCTGGCCGCCGCCCTG
CCAAGGCACATGACCGCCGATCGGATGATCAGAATCGTGACCACA
GAGATCCGCAAGACACCCGCCCTGGCCCAGTGCGACCAGAGCTCC
TTCATCGGAGCAGTGGTGCAGTGTAGCCAGCTGGGCCTGGAGCCT
GGCTCCGCCCTGGGCCACGCCTACCTGCTGCCATTTGGCAACGGC
CGGTCCAAGTCTGGCCAGAGCAATGTGCAGCTGATCATCGGCTAT
AGAGGCATGATCGATCTGGCCCGGAGATCCGGACAGATCGTGAGC
CTGTCCGCCAGGGTGGTGCGCGCAGACGATGAGTTCTCTTTTGAG
TACGGCCTGGATGAGAACCTGATCCACCGGCCAGGCGACAATGAG
TCCGCCCCCATCACCCACGTGTATGCAGTGGCAAGACTGAAGGAC
GGAGGCACCCAGTTCGAAGTGATGACAGCCAAGCAGGTGGAGAAG
GTGAAGGCCCAGTCCAAGGCCTCTAGCAACGGACCCTGGGTGACC
CACTGGGAGGAGATGGCCAAGAAAACCGTGATCAGGCGCCTGTTT
AAGTACCTGCCCGTGAGCATCGAGATGCAGAAGGCCGTGGTGCTG
GACGAGAAGGCCGAGAGCGACGTGGATCAGGACAATGCCTCTGTG
CTGAGCGCCGAGTATTCCGTGCTGGAGTCTGGCACAGGCGAG
Pantoea dispersa RecE DNA
(SEQ ID NO: 90):
GAGCCAGGCATCTACTATGACATCAGCAACGAGGCCTACCACTCC
GGCCCCGGCATCAGCAAGTCCCAGCTGGACGACATCGCCAGGAGC
CCTGCCATCTTCCAGTGGCGCAAGGACGCCCCAGTGGATACCGAG
AAAACCAAGGCCCTGGACCTGGGCACCGATTTCCACTGCGCCGTG
CTGGAGCCAGAGAGGTTTGCAGACATGTATCGCGTGGGCCCTGAA
GTGAATCGGAGAACCACAGCCGGCAAGGCCGAGGAGAAGGAGTTC
TTTGAGAAGTGTGAGAAGGATGGAGCCGTGCCCATCACCCACGAC
GATGCACGGAAGGTGGAGCTGATGAGAGGCTCCGTGATGGCCCAC
CCTATCGCCAAGCAGATGATCGCAGCACAGGGACACGCAGAGGCC
TCTATCTACTGGCACGACGAGAGCACAGGCAACCTGTGCCGGTGT
AGACCCGACAAGTTTATCCCTGATTGGAATTGGATCGTGGACGTG
AAAACCACAGCCGATATGAAGAAGTTCAGGCGCGAGTTTTACGAT
CTGCGGTATCACGTGCAGGACGCCTTCTACACCGATGGCTATGCC
GCCCAGTTTGGCGAGCGGCCTACCTTCGTGTTTGTGGTGACATCC
ACCACAATCGACTGCGGCAGATACCCCACCGAGGTGTTCTTTCTG
GATGAGGAGACAAAGGCCGCCGGCAGGTCTGAGTACCAGAGCAAC
CTGGTGACCTATTCCGAGTGTCTGTCTCGCAATGAGTGGCCAGGC
ATCGCCACACTGTCTCTGCCCCACTGGGCCAAGGAGCTGAGGAAC
GTG
Type-F symbiont of Plautia stali RecT DNA
(SEQ ID NO: 91):
TCCAACCAGCCCCCTATCGCCTCTGCCGATCTGCAGAAAACCCAG
CAGTCTAAGCAGGTGGCCAACAAGACCCCTGAGCAGACACTGGTG
GGCTTCATGAATCAGCCAGCAATGAAGTCCCAGCTGGCCGCCGCC
CTGCCAAGGCACATGACAGCCGATCGGATGATCAGAATCGTGACC
ACAGAGATCCGCAAGACCCCCGCCCTGGCCACATGCGACCAGAGC
TCCTTCATCGGAGCAGTGGTGCAGTGTAGCCAGCTGGGCCTGGAG
CCTGGCTCCGCCCTGGGCCACGCCTACCTGCTGCCATTTGGCAAC
GGCCGGTCCAAGTCTGGCCAGTCTAATGTGCAGCTGATCATCGGC
TATAGAGGCATGATCGACCTGGCCCGGAGAAGCGGACAGATCGTG
AGCCTGTCCGCCAGGGTGGTGCGCGCAGACGATGAGTTCTCCTTT
GAGTACGGCCTGGATGAGAACCTGATCCACCGGCCAGGCGATAAT
GAGGACGCCCCCATCACCCACGTGTATGCAGTGGCAAGACTGAAG
GACGGAGGCACCCAGTTCGAAGTGATGACAGCCAAGCAGGTGGAG
AAGGTGAAGGCCCAGAGCAAGGCCTCTAGCAACGGACCCTGGGTG
ACCCACTGGGAGGAGATGGCCAAGAAAACCGTGATCAGGCGCCTG
TTTAAGTACCTGCCCGTGAGCATCGAGATGCAGAAGGCCGTGGTG
CTGGATGAGAAGGCCGAGAGCGACGTGGATCAGGACAATGCCTCT
GTGCTGAGCGCCGAGTATTCCGTGCTGGAGGGCGACGGCGGCGAG
Type-F symbiont of Plautia stali RecE DNA
(SEQ ID NO: 92):
CAGCCTGGCATCTACTATGACATCAGCAACGAGGATTATCACGGC
GGCCCTGGCATCAGCAAGTCCCAGCTGGACGACATCGCCATCTCC
CCAGCCATCTACCAGTGGAGGAAGCACGCCCCCGTGGACGAGGAG
AAAACCGCCGCCCTGGATCTGGGCACAGCCCTGCACTGCCTGCTG
CTGGAGCCTGACGAGTTCTCTAAGAGATTTGAGATCGGCCCAGAG
GTGAACCGGAGAACCACAGCCGGCAAGGAGAAGGAGAAGGAGTTC
ATGGAGAGGTGTGAGGCAGAGGGAGTGACCCCTATCACACACGAC
GATAATCGGAAGCTGAGACTGATGAGGGATAGCGCAATGGCCCAC
CCAATCGCCAGATGGATGCTGGAGGCACAGGGAAACGCAGAGGCC
TCTATCTATTGGAATGACAGGGATACCGGCGTGCTGAGCAGGTGC
CGCCCCGACAAGATCATCACCGACTTCAACTGGTGCGTGGACGTG
AAGTCCACAGCCGACATCATCAAGTTCCAGAAGGACTTTTACTCT
TATCGCTACCACGTGCAGGACGCCTTCTATTCCGATGGCTACGAG
TCTCACTTTGACGAGACACCAACATTCGCCTTTCTGGCCGTGTCT
ACAAGCATCGATTGCGGCCGGTATCCCGTGCAGGTGTTCATCATG
GACCAGCAGGCAAAGGATGCAGGAAGGGCCGAGTACAAGCGGAAC
ATCCACACCTTTGCCGAGTGTCTGAGCCGCAATGAGTGGCCTGGC
ATCGCCACACTGTCCCTGCCTTACTGGGCCAAGGAGCTGCGGAAT
GAG
Providencia stuartii RecT DNA
(SEQ ID NO: 93):
AGCAACCCACCTCTGGCCCAGGCAGACCTGCAGAAAACCCAGGGC
ACAGAGGTGAAGGAGAAAACCAAGGATCAGATGCTGGTGGAGCTG
ATCAATAAGCCTTCCATGAAGGCACAGCTGGCCGCCGCCCTGCCA
AGGCACATGACACCCGACCGGATGATCAGAATCGTGACCACAGAG
ATCAGAAAGACCCCCGCCCTGGCCACATGCGATATGCAGAGCTTC
GTGGGAGCAGTGGTGCAGTGTTCCCAGCTGGGCCTGGAGCCTGGC
AACGCCCTGGGACACGCCTACCTGCTGCCTTTTGGCAACGGCAAG
TCTAAGAGCGGCCAGTCTAATGTGCAGCTGATCATCGGCTATCGG
GGCATGATCGACCTGGCCCGGAGAAGCGGCCAGATCGTGTCCATC
TCTGCCAGGACCGTGCGCCAGGGCGATAACTTCCACTTTGAGTAC
GGCCTGAACGAGAATCTGACCCACGTGCCTGGCGAGAATGAGGAC
TCTCCAATCACACACGTGTACGCAGTGGCAAGGCTGAAGGATGGA
GGCGTGCAGTTCGAAGTGATGACCTATAACCAGATCGAGAAGGTG
CGCGCCAGCTCCAAGGCAGGACAGAATGGACCCTGGGTGAGCCAC
TGGGAGGAGATGGCCAAGAAAACCGTGATCAGGCGCCTGTTCAAG
TACCTGCCCGTGTCTATCGAGATGCAGAAGGCCGTGATCCTGGAC
GAGAAGGCCGAGGCCAACATCGATCAGGAGAATGCCACCATCTTT
GAGGGCGAGTATGAGGAAGTGGGCACAGACGGCAAG
Providencia stuartii RecE DNA
(SEQ ID NO: 94):
GAGGGCATCTACTATAACATCAGCAATGAGGACTACCACAACGGC
CTGGGCATCTCCAAGTCTCAGCTGGATCTGATCAATGAGATGCCT
GCCGAGTATATCTGGTCCAAGGAGGCCCCCGTGGACGAGGAGAAG
ATCAAGCCTCTGGAGATCGGCACCGCCCTGCACTGCCTGCTGCTG
GAGCCAGACGAGTACCACAAGAGATATAAGATCGGCCCCGATGTG
AACCGGAGAACAAATGCCGGCAAGGAGAAGGAGAAGGAGTTCTTT
GATATGTGCGAGAAGGAGGGCATCACCCCCATCACACACGACGAT
AACCGGAAGCTGATGATCATGAGAGACTCTGCCCTGGCCCACCCT
ATCGCCAAGTGGTGTCTGGAGGCCGATGGCGTGAGCGAGAGCTCC
ATCTACTGGACCGACAAGGAGACAGATGTGCTGTGCAGGTGTCGC
CCAGACCGCATCATCACCGCCCACAACTACATCGTGGATGTGAAG
TCTAGCGGCGACATCGAGAAGTTCGATTACGAGTACTACAACTAC
AGATACCACGTGCAGGACGCCTTTTACTCCGATGGCTATAAGGAG
GTGACCGGCATCACCCCTACATTCCTGTTTCTGGTGGTGTCTACC
AAGATCGACTGCGGCAAGTACCCCGTGCGGACCTACGTGATGAGC
GAGGAGGCAAAGTCCGCCGGAAGGACCGCCTACAAGCACAACCTG
CTGACCTATGCCGAGTGTCTGAAAACCGATGAGTGGGCCGGCATC
AGGACACTGTCTCTGCCCAGATGGGCAAAGGAGCTGCGGAATGAG
Providencia sp. MGF014 RecT DNA
(SEQ ID NO: 95):
TCTAACCCCCCTCTGGCCCAGAGCGACCTGCAGAAAACCCAGGGC
ACAGAGGTGAAGGTGAAAACCAAGGATCAGCAGCTGATCCAGTTC
ATCAATCAGCCTTCTATGAAGGCACAGCTGGCCGCCGCCCTGCCA
AGGCACATGACACCCGACCGGATGATCAGAATCGTGACCACAGAG
ATCAGAAAGACCCCCGCCCTGGCCACATGCGATATGCAGTCCTTC
GTGGGCGCCGTGGTGCAGTGTTCTCAGCTGGGCCTGGAGCCTGGC
AACGCCCTGGGACACGCCTACCTGCTGCCTTTTGGCAACGGCAAG
GCCAAGTCCGGCCAGTCTAATGTGCAGCTGATCATCGGCTATCGG
GGCATGATCGACCTGGCCCGGAGATCCAACCAGATCATCTCTATC
AGCGCCAGGACCGTGCGCCAGGGCGATAACTTCCACTTTGAGTAC
GGCCTGAATGAGGACCTGACCCACACACCTAGCGAGAATGAGGAT
TCCCCAATCACCCACGTGTACGCAGTGGCAAGGCTGAAGGACGGA
GGCGTGCAGTTTGAAGTGATGACATATAACCAGGTGGAGAAGGTG
CGCGCCAGCTCCAAGGCAGGACAGAATGGACCCTGGGTGAGCCAC
TGGGAGGAGATGGCCAAGAAAACCGTGATCAGGCGCCTGTTCAAG
TACCTGCCCGTGTCCATCGAGATGCAGAAGGCAGTGGTGCTGGAC
GAGAAGGCAGAGGCCAACGTGGATCAGGAGAATGCCACCATCTTT
GAGGGCGAGTATGAGGAAGTGGGCACAGATGGCAAT
Providencia sp. MGF014 RecE DNA
(SEQ ID NO: 96):
AAGGAGGGCATCTACTATAACATCAGCAATGAGGACTACCACAAC
GGCCTGGGCATCTCCAAGTCTCAGCTGGATCTGATCAATGAGATG
CCTGCCGAGTATATCTGGTCCAAGGAGGCCCCCGTGGACGAGGAG
AAGATCAAGCCTCTGGAGATCGGCACCGCCCTGCACTGCCTGCTG
CTGGAGCCAGACGAGTACCACAAGAGATATAAGATCGGCCCCGAT
GTGAACCGGAGAACAAATGTGGGCAAGGAGAAGGAGAAGGAGTTC
TTTGATATGTGCGAGAAGGAGGGCATCACCCCCATCACACACGAC
GATAACCGGAAGCTGATGATCATGAGAGACTCTGCCCTGGCCCAC
CCTATCGCCAAGTGGTGTCTGGAGGCCGATGGCGTGAGCGAGAGC
TCCATCTACTGGACCGACAAGGAGACAGATGTGCTGTGCAGGTGT
CGCCCAGACCGCATCATCACCGCCCACAACTACATCATCGATGTG
AAGTCTAGCGGCGACATCGAGAAGTTCGATTACGAGTACTACAAC
TACAGATACCACGTGCAGGACGCCTTTTACTCCGATGGCTATAAG
GAGGTGACCGGCATCACCCCTACATTCCTGTTTCTGGTGGTGTCT
ACCAAGATCGACTGCGGCAAGTACCCCGTGCGGACCTACGTGATG
AGCGAGGAGGCAAAGTCCGCCGGAAGGACCGCCTACAAGCACAAC
CTGCTGACCTATGCCGAGTGTCTGAAAACCGATGAGTGGGCCGGC
ATCAGGACACTGTCTCTGCCCAGATGGGCAAAGGAGCTGCGGAAT
GAG
Shewanella putrefaciens RecT DNA
(SEQ ID NO: 97):
CAGACCGCACAGGTGAAGCTGAGCGTGCCCCACCAGCAGGTGTAC
CAGGACAACTTCAATTATCTGAGCTCCCAGGTGGTGGGCCACCTG
GTGGATCTGAACGAGGAGATCGGCTACCTGAACCAGATCGTGTTT
AATTCTCTGAGCACCGCCTCTCCCCTGGACGTGGCAGCACCTTGG
AGCGTGTACGGCCTGCTGCTGAACGTGTGCCGGCTGGGCCTGTCC
CTGAATCCAGAGAAGAAGCTGGCCTATGTGATGCCCTCCTGGTCT
GAGACAGGCGAGATCATCATGAAGCTGTACCCCGGCTATAGGGGC
GAGATCGCCATCGCCTCTAACTTCAATGTGATCAAGAACGCCAAT
GCCGTGCTGGTGTATGAGAACGATCACTTCCGCATCCAGGCAGCA
ACCGGCGAGATCGAGCACTTTGTGACAAGCCTGTCCATCGACCCT
AGGGTGCGCGGAGCATGCAGCGGAGGCTACTGTCGGTCCGTGCTG
ATGGATAATACAATCCAGATCTCTTATCTGAGCATCGAGGAGATG
AACGCCATCGCCCAGAATCAGATCGAGGCCAACATGGGCAATACC
CCTTGGAACTCCATCTGGCGGACAGAGATGAATAGAGTGGCCCTG
TACCGGAGAGCAGCAAAGGACTGGAGGCAGCTGATCAAGGCCACC
CCAGAGATCCAGTCCGCCCTGTCTGATACAGAGTAT
Shewanella putrefaciens RecE DNA
(SEQ ID NO: 98):
GGCACCGCCCTGGCCCAGACAATCAGCCTGGACTGGCAGGATACC
ATCCAGCCAGCATACACAGCCTCCGGCAAGCCTAACTTCCTGAAT
GCCCAGGGCGAGATCGTGGAGGGCATCTACACCGATCTGCCTAAT
TCCGTGTATCACGCCCTGGACGCACACAGCTCCACCGGCATCAAG
ACATTCGCCAAGGGCCGCCACCACTACTTTCGGCAGTATCTGTCT
GACGTGTGCCGGCAGAGAACAAAGCAGCAGGAGTACACCTTCGAC
GCCGGCACCTACGGCCACATGCTGGTGCTGGAGCCAGAGAACTTC
CACGGCAACTTCATGAGGAACCCCGTGCCTGACGATTTTCCAGAC
ATCGAGCTGATCGAGAGCATCCCACAGCTGAAGGCCGCCCTGGCC
AAGAGCAACCTGCCCGTGTCCGGAGCAAAGGCCGCCCTGATCGAG
AGACTGTACGCCTTCGACCCATCCCTGCCCCTGTTTGAGAAGATG
AGGGAGAAGGCCATCACCGACTATCTGGATCTGCGCTACGCCAAG
TATCTGCGGACCGACGTGGAGCTGGATGAGATGGCCACATTCTAC
GGCATCGATACCTCTCAGACACGGGAGAAGAAGATCGAGGAGATC
CTGGCCATCTCTCCTAGCCAGCCAATCTGGGAGAAGCTGATCAGC
CAGCACGTGATCGACCACATCGTGTGGGACGATGCCATGAGGGTG
GAGAGATCCACCAGGGCCCACCCTAAGGCAGACTGGCTGATCTCT
GATGGCTATGCCGAGCTGACAATCATCGCAAGGTGCCCAACCACC
GGCCTGCTGCTGAAGGTGCGGTTTGACTGGCTGAGGAATGATGCC
ATCGGCGTGGACTTCAAGACCACACTGTCTACCAACCCCACAAAG
TTTGGCTACCAGATCAAGGACCTGCGGTATGATCTGCAGCAGGTG
TTCTACTGTTATGTGGCCAATCTGGCCGGCATCCCTGTGAAGCAC
TTCTGCTTTGTGGCCACCGAGTACAAGGACGCCGATAACTGTGAG
ACATTTGAGCTGTCTCACAAGAAAGTGATCGAGAGCACCGAGGAG
ATGTTCGACCTGCTGGATGAGTTTAAGGAGGCCCTGACCTCCGGC
AATTGGTATGGCCACGACAGGTCCCGCTCTACATGGGTCATCGAG
GTG
Bacillus sp. MUM 116 RecT DNA
(SEQ ID NO: 99):
AGCAAGCAGCTGACCACAGTGAATACCCAGGCCGTGGTGGGCACA
TTCTCCCAGGCCGAGCTGGATACCCTGAAGCAGACAATCGCCAAG
GGCACCACAAACGAGCAGTTCGCCCTGTTTGTGCAGACCTGCGCC
AACTCTAGGCTGAATCCATTTCTGAACCACATCCACTGTATCGTG
TATAACGGCAAGGAGGGCGCCACCATGAGCCTGCAGATCGCAGTG
GAGGGCATCCTGTACCTGGCACGCAAGACAGACGGCTATAAGGGC
ATCGAGTGCCAGCTGATCCACGAGAATGACGAGTTCAAGTTTGAT
GCCAAGTCCAAGGAGGTGGATCACCAGATCGGATTCCCCAGGGGC
AACGTGATCGGAGGATATGCAATCGCAAAGAGGGAGGGCTTTGAC
GATGTGGTGGTGCTGATGGAGTCTAACGAGGTGGACCACATGCTG
AAGGGCCGGAATGGCCACATGTGGAGAGACTGGTTCAACGATATG
TTTAAGAAGCACATCATGAAGCGGGCCGCCAAGCTGCAGTACGGC
ATCGAGATCGCAGAGGACGAGACAGTGAGCAGCGGACCTAGCGTG
GATAATATCCCAGAGTATAAGCCACAGCCCCGGAAGGACATCACA
CCCAACCAGGACGTGATCGATGCCCCCCCTCAGCAGCCTAAGCAG
GACGATGAGGCCGCCAAGCTGAAGGCCGCCAGATCTGAGGTGAGC
AAGAAGTTCAAGAAGCTGGGCATCGTGAAGGAGGATCAGACCGAG
TACGTGGAGAAGCACGTGCCTGGCTTCAAGGGCACACTGTCCGAC
TTTATCGGCCTGTCTCAGCTGCTGGATCTGAATATCGAGGCCCAG
GAGGCCCAGTCCGCCGACGGCGATCTGCTGGAC
Bacillus sp. MUM 116 RecE DNA
(SEQ ID NO: 100):
ACCTACGCCGCCGACGAGACACTGGTGCAGCTGCTGCTGTCCGTG
GATGGCAAGCAGCTGCTGCTGGGAAGGGGCCTGAAGAAGGGCAAG
GCCCAGTACTATATCAATGAGGTGCCATCTAAGGCCAAGGAGTTC
GAGGAGATCCGGGACCAGCTGTTTGACAAGGATCTGTTCATGTCC
CTGTTTAACCCCTCTTACTTCTTTACCCTGCACTGGGAGAAGCAG
AGGGCCATGATGCTGAAGTATGTGACAGCCCCCGTGTCTAAGGAG
GTGCTGAAGAATCTGCCTGAGGCCCAGTCCGAGGTGCTGGAGAGA
TACCTGAAGAAGCACTCTCTGGTGGATCTGGAGAAGATCCACAAG
GACAACAAGAATAAGCAGGATAAGGCCTATATCTCTGCCCAGAGC
AGGACCAACACACTGAAGGAGCAGCTGATGCAGCTGACCGAGGAG
AAGCTGGACATCGATTCCATCAAGGCCGAGCTGGCCCACATCGAC
ATGCAGGTCATCGAGCTGGAGAAGCAGATGGATACAGCCTTCGAG
AAGAACCAGGCCTTTAATCTGCAGGCCCAGATCAGGAATCTGCAG
GACAAGATCGAGATGAGCAAGGAGCGGTGGCCCTCCCTGAAGAAC
GAAGTGATCGAGGATACCTGCCGGACATGCAAGCGGCCCCTGGAC
GAGGATAGCGTGGAGGCCGTGAAGGCCGACAAGGATAATCGGATC
GCCGAGTACAAGGCCAAGCACAACTCCCTGGTGTCTCAGAGAAAT
GAGCTGAAGGAGCAGCTGAACACCATCGAGTATATCGACGTGACA
GAGCTGAGAGAGCAGATCAAGGAGCTGGATGAGTCCGGACAGCCT
CTGAGGGAGCAGGTGCGCATCTACAGCCAGTATCAGAATCTGGAC
ACCCAGGTGAAGTCCGCCGAGGCAGACGAGAACGGCATCCTGCAG
GATCTGAAGGCCTCTATCTTCATCCTGGATAGCATCAAGGCCTTT
AGGGGCAAGGAGGCCGAGATGCAGGCCGAGAAGGTGCAGGCCCTG
TTCACCACACTGAGCGTGCGCCTGTTTAAGCAGAATAAGGGCGAC
GGCGAGATCAAGCCAGATTTCGAGATCGAGATGAACGACAAGCCC
TATCGGACCCTGAGCCTGTCCGAGGGCATCCGGGCAGGCCTGGAG
CTGCGGGACGTGCTGAGCCAGCAGTCCGAGCTGGTGACCCCTACA
TTCGTGGATAATGCCGAGTCTATCACCAGCTTCAAGCAGCCAAAC
GGCCAGCTGATCATCAGCCGGGTGGTGGCAGGACAGGAGCTGAAG
ATCGAGGCCGTGAGCGAG
Shigella sonnei RecT DNA
(SEQ ID NO: 101):
ACCAAGCAGCCCCCTATCGCCAAGGCCGACCTGCAGAAAACCCAG
GAGAACAGGGCACCAGCAGCCATCAAGAACAATGATGTGATCTCC
TTTATCAATCAGCCCTCTATGAAGGAGCAGCTGGCCGCCGCCCTG
CCTAGGCACATGACCGCCGAGAGGATGATCCGCATCGCCACCACA
GAGATCCGCAAGGTGCCTGCCCTGGGCAACTGCGACACAATGAGC
TTCGTGAGCGCCATCGTGCAGTGTAGCCAGCTGGGCCTGGAGCCA
GGCTCCGCCCTGGGCCACGCCTACCTGCTGCCCTTCGGCAACAAG
AATGAGAAGTCCGGCAAGAAGAATGTGCAGCTGATCATCGGCTAT
AGGGGCATGATCGATCTGGCCCGGAGATCTGGCCAGATCGCCTCT
CTGAGCGCCAGAGTGGTGCGGGAGGGCGACGAGTTCAACTTTGAG
TTCGGCCTGGATGAGAAGCTGATCCACCGGCCTGGCGAGAATGAG
GACGCCCCAGTGACCCACGTGTACGCAGTGGCCAGACTGAAGGAT
GGCGGCACCCAGTTTGAAGTGATGACAAGGCGCCAGATCGAGCTG
GTGAGGTCCCAGTCTAAGGCCGGCAACAATGGCCCTTGGGTGACC
CACTGGGAGGAGATGGCCAAGAAAACCGCCATCCGGAGACTGTTC
AAGTACCTGCCAGTGTCTATCGAGATCCAGCGCGCCGTGAGCATG
GACGAGAAGGAGCCACTGACCATCGACCCCGCCGATAGCTCCGTG
CTGACAGGCGAGTATTCTGTGATCGATAACAGCGAGGAG
ShtgcllascnnctRccEDNA(SEQIDNQ:102):GATCGCGGCC
TGCTGACAAAGGAGTGGAGGAAGGGAAACCGGGTGAGCCGGATCA
CCAGGACAGCCAGCGGAGCAAACGCAGGAGGAGGAAATCTGACCG
ACAGAGGCGAGGGCTTCGTGCACGATCTGACAAGCCTGGCCCGCG
ACATCGCAACCGGCGTGCTGGCCCGGAGCATGGACGTGGACATCT
ACAACCTGCACCCTGCCCACGCCAAGAGGATCGAGGAGATCATCG
CCGAGAATAAGCCCCCTTTCAGCGTGTTTAGAGACAAGTTTATCA
CAATGCCAGGCGGCCTGGACTACTCCAGGGCCATCGTGGTGGCCT
CTGTGAAGGAGGCCCCAATCGGCATCGAAGTGATCCCCGCCCACG
TGACCGCCTATCTGAACAAGGTGCTGACCGAGACAGACCACGCCA
ATCCAGATCCCGAGATCGTGGACATCGCATGCGGCAGAAGCTCCG
CCCCTATGCCACAGAGGGTGACCGAGGAGGGCAAGCAGGACGATG
AGGAGAAGCTGCAGCCTTCTGGCACCACAGCAGATGAGCAGGGAG
AGGCAGAGACAATGGAGCCAGACGCCACAAAGCACCACCAGGATA
CCCAGCCTCTGGACGCCCAGAGCCAGGTGAACAGCGTGGATGCCA
AGTATCAGGAGCTGAGAGCCGAGCTGCACGAGGCCAGGAAGAACA
TCCCTTCCAAGAATCCAGTGGACGCAGATAAGCTGCTGGCCGCCT
CTCGCGGCGAGTTCGTGGACGGCATCAGCGACCCAAACGATCCCA
AGTGGGTGAAGGGCATCCAGACACGGGATTCCGTGTACCAGAATC
AGCCTGAGACAGAGAAAACCAGCCCCGACATGAAGCAGCCAGAGC
CTGTGGTGCAGCAGGAGCCTGAGATCGCCTTCAACGCCTGCGGAC
AGACCGGCGGCGACAATTGCCCAGATTGTGGCGCCGTGATGGGCG
ATGCCACCTATCAGGAGACATTTGACGAGGAGAACCAGGTGGAGG
CCAAGGAGAATGATCCTGAGGAGATGGAGGGCGCCGAGCACCCAC
ACAACGAGAATGCCGGCAGCGACCCCCACAGAGACTGTTCCGATG
AGACAGGCGAGGTGGCCGATCCCGTGATCGTGGAGGACATCGAGC
CTGGCATCTACTATGGCATCAGCAACGAGAATTACCACGCAGGCC
CCGGCGTGTCCAAGTCTCAGCTGGACGACATCGCCGACACACCTG
CCCTGTATCTGTGGAGGAAGAACGCCCCAGTGGATACCACAAAGA
CCAAGACACTGGACCTGGGCACCGCATTCCACTGCCGCGTGCTGG
AGCCAGAGGAGTTCAGCAATCGGTTTATCGTGGCCCCCGAGTTCA
ACCGGAGAACAAATGCCGGCAAGGAGGAGGAGAAGGCCTTTCTGA
TGGAGTGTGCCTCCACAGGCAAGATGGTCATCACCGCCGAGGAGG
GCAGAAAGATCGAGCTGATGTACCAGTCTGTGATGGCACTGCCAC
TGGGACAGTGGCTGGTGGAGAGCGCCGGACACGCAGAGTCTAGCA
TCTATTGGGAGGACCCCGAGACAGGCATCCTGTGCAGGTGTCGCC
CCGACAAGATCATCCCTGAGTTCCACTGGATCATGGACGTGAAAA
CCACAGCCGACATCCAGCGGTTCAAGACAGCCTACTATGATTACA
GGTATCACGTGCAGGATGCCTTCTACTCCGACGGCTATGAGGCCC
AGTTTGGCGTGCAGCCCACCTTCGTGTTTCTGGTGGCCTCTACCA
CAATCGAGTGCGGCAGATACCCCGTGGAGATCTTTATGATGGGAG
AGGAGGCAAAGCTGGCCGGACAGCTGGAGTATCACCGCAACCTGC
GGACACTGGCCGATTGTCTGAATACCGACGAGTGGCCAGCCATCA
AGACCCTGTCCCTGCCCAGATGGGCAAAGGAGTACGCCAACGAC
Salmonella enterica RecT DNA
(SEQ ID NO: 103):
ACCAAGCAGCCCCCTATCGCCAAGGCCGACCTGCAGAAAACCCAG
GGAAACAGGGCACCTGCAGCAGTGAATGACAAGGATGTGCTGTGC
GTGATCAACAGCCCTGCCATGAAGGCACAGCTGGCCGCCGCCCTG
CCAAGGCACATGACCGCCGAGAGGATGATCCGCATCGCCACCACA
GAGATCAGGAAGGTGCCAGAGCTGCGCAACTGCGACAGCACCAGC
TTCATCGGCGCCATCGTGCAGTGTTCTCAGCTGGGCCTGGAGCCC
GGCAGCGCCCTGGGCCACGCCTACCTGCTGCCTTTTGGCAATGGC
AAGGCCAAGAACGGCAAGAAGAATGTGCAGCTGATCATCGGCTAT
CGGGGCATGATCGATCTGGCCCGGAGATCTGGCCAGATCATCTCC
CTGAGCGCCAGAGTGGTGCGGGAGTGTGACGAGTTCTCCTACGAG
CTGGGCCTGGATGAGAAGCTGGTGCACCGGCCAGGCGAGAACGAG
GACGCACCCATCACCCACGTGTATGCCGTGGCCAAGCTGAAGGAT
GGCGGCGTGCAGTTTGAAGTGATGACCAAGAAGCAGGTGGAGAAG
GTGAGAGATACACACTCCAAGGCCGCCAAGAATGCCGCCTCTAAG
GGCGCCAGCTCCATCTGGGACGAGCACTTCGAGGATATGGCCAAG
AAAACCGTGATCCGGAAGCTGTTTAAGTACCTGCCCGTGAGCATC
GAGATCCAGAGAGCCGTGAGCATGGACGGCAAGGAGGTGGAGACA
ATCAACCCAGACGACATCAGCGTGATCGCCGGCGAGTATTCCGTG
ATCGATAATCCCGAGGAG
Salmonella enterica RecE DNA
(SEQ ID NO: 104):
GATCGCGGCCTGCTGACAAAGGAGTGGAGGAAGGGAAACCGGGTG
AGCCGGATCACCAGGACAGCCAGCGGAGCAAACGCAGGAGGAGGA
AATCTGACCGACAGAGGCGAGGGCTTCGTGCACGATCTGACAAGC
CTGGCCCGCGACGTGGCAACCGGCGTGCTGGCCCGGAGCATGGAC
GTGGACATCTACAACCTGCACCCTGCCCACGCCAAGAGGGTGGAG
GAGATCATCGCCGAGAATAAGCCCCCTTTCAGCGTGTTTAGAGAC
AAGTTTATCACAATGCCTGGCGGCCTGGACTACTCCAGGGCCATC
GTGGTGGCCTCTGTGAAGGAGGCCCCTATCGGCATCGAAGTGATC
CCAGCCCACGTGACCGAGTATCTGAACAAGGTGCTGACCGAGACA
GACCACGCCAATCCAGATCCCGAGATCGTGGACATCGCATGCGGC
AGAAGCTCCGCCCCTATGCCACAGAGGGTGACCGAGGAGGGCAAG
CAGGACGATGAGGAGAAGCCCCAGCCTTCTGGAGCTATGGCCGAC
GAGCAGGCAACCGCAGAGACAGTGGAGCCAAACGCCACAGAGCAC
CACCAGAATACCCAGCCCCTGGATGCCCAGAGCCAGGTGAACTCC
GTGGACGCCAAGTATCAGGAGCTGAGAGCCGAGCTGCAGGAGGCC
AGGAAGAACATCCCCTCCAAGAATCCTGTGGACGCAGATAAGCTG
CTGGCCGCCTCTCGCGGCGAGTTCGTGGATGGCATCAGCGACCCT
AACGATCCAAAGTGGGTGAAGGGCATCCAGACACGGGATTCCGTG
TACCAGAATCAGCCCGAGACAGAGAAGATCTCTCCTGACGCCAAG
CAGCCAGAGCCCGTGGTGCAGCAGGAGCCCGAGACAGTGTGCAAC
GCCTGTGGACAGACCGGCGGCGACAATTGCCCTGATTGTGGCGCC
GTGATGGGCGACGCCACATATCAGGAGACATTCGGCGAGGAGAAT
CAGGTGGAGGCCAAGGAGAAGGACCCCGAGGAGATGGAGGGAGCA
GAGCACCCTCACAACGAGAATGCCGGCAGCGACCCACACAGAGAC
TGTTCCGATGAGACAGGCGAGGTGGCCGATCCAGTGATCGTGGAG
GACATCGAGCCTGGCATCTACTATGGCATCAGCAACGAGAATTAC
CACGCAGGCCCCGGCGTGTCCAAGTCTCAGCTGGACGACATCGCC
GACACACCCGCCCTGTATCTGTGGAGGAAGAACGCCCCTGTGGAT
ACCACAAAGACCAAGACACTGGACCTGGGCACCGCATTCCACTGC
CGCGTGCTGGAGCCTGAGGAGTTCAGCAATCGGTTTATCGTGGCC
CCAGAGTTCAACCGGAGAACAAATGCCGGCAAGGAGGAGGAGAAG
GCCTTTCTGATGGAGTGTGCCTCCACCGGCAAGACAGTGATCACC
GCCGAGGAGGGCAGAAAGATCGAGCTGATGTACCAGTCTGTGATG
GCACTGCCTCTGGGACAGTGGCTGGTGGAGAGCGCCGGACACGCA
GAGTCTAGCATCTATTGGGAGGACCCCGAGACAGGCATCCTGTGC
AGGTGTCGCCCAGACAAGATCATCCCCGAGTTCCACTGGATCATG
GACGTGAAAACCACAGCCGACATCCAGCGGTTCAAGACAGCCTAC
TATGATTACAGGTATCACGTGCAGGATGCCTTCTACTCCGACGGC
TATGAGGCCCAGTTTGGCGTGCAGCCAACCTTCGTGTTTCTGGTG
GCCTCTACCACAGTGGAGTGCGGCAGATACCCCGTGGAGATCTTT
ATGATGGGAGAGGAGGCAAAGCTGGCCGGACAGCAGGAGTATCAC
CGCAACCTGCGGACACTGGCCGATTGTCTGAATACCGACGAGTGG
CCTGCCATCAAGACCCTGTCCCTGCCACGGTGGGCCAAGGAGTAC
GCCAACGAC
Acetobacter RecT DNA
(SEQ ID NO: 105):
AACGCCCCCCAGAAGCAGAATACCAGAGCCGCCGTGAAGAAGATC
AGCCCTCAGGAGTTCGCCGAGCAGTTTGCCGCCATCATCCCACAG
GTGAAGTCCGTGCTGCCCGCCCACGTGACCTTCGAGAAGTTTGAG
CGGGTGGTGAGACTGGCCGTGCGGAAGAACCCTGACCTGCTGACA
TGCTCCCCAGCCTCTCTGTTCATGGCATGTATCCAGGCAGCCTCC
GACGGCCTGCTGCCTGATGGAAGGGAGGGAGCAATCGTGAGCCGG
TGGAGCTCCAAGAAGAGCTGCAACGAGGCCTCCTGGATGCCAATG
GTGGCCGGCCTGATGAAGCTGGCCCGGAACAGCGGCGACATCGCC
AGCATCTCTAGCCAGGTGGTGTTCGAGGGCGAGCACTTTAGAGTG
GTGCTGGGCGACGAGGAGAGGATCGAGCACGAGCGCGATCTGGGC
AAGACCGGCGGCAAGATCGTGGCAGCCTACGCCGTGGCAAGGCTG
AAGGACGGCAGCGATCCAATCCGCGAGATCATGTCCTGGGGCCAG
ATCGAGAAGATCAGAAACACAAATAAGAAGTGGGAGTGGGGACCC
TGGAAGGCCTGGGAGGACGAGATGGCCAGAAAGACCGTGATCCGG
AGACTGGCCAAGAGACTGCCCATGTCTACAGATAAGGAGGGAGAG
AGGCTGCGCAGCGCCATCGAGAGGATCGACTCCCTGGTGGACATC
TCTGCCAACGTGGACGCACCTCAGATCGCAGCAGACGATGAGTTT
GCCGCCGCCGCCCACGGCGTGGAGCCACAGCAGATCGCAGCACCT
GACCTGATCGGCCGCCTGGCCCAGATGCAGTCCCTGGAGCAGGTG
CAGGACATCGAGCCCCAGGTGTCTCACGCCATCCAGGAGGCCGAC
AAGAGGGGCGACAGCGATACAGCCAATGCCCTGGATGCCGCCCTG
CAGAGCGCCCTGTCCCGCACCTCTACAGCCAAGGAGGAGGTGCCT
GCC
Acetobacter RecE DNA
(SEQ ID NO: 106):
GTGATCTCTAAGAGCGGCATCTACGACCTGACCAACGAGCAGTAT
CACGCCGATCCTTGCCCAGAGATGTCCCTGAGCTCCTCTGGAGCC
AGGGACCTGCTGAGCTCCTGTCCTGCCAAGTTCATCGCCGCCAAG
CAGCTGCCACAGCAGAATAAGAGGTGCTTTGACATCGGCTCTGCC
GGACACCTGATGGTGCTGGAGCCACACCTGTTCGACCAGAAGGTG
TGCGAGATCAAGCACCCTGATTGGCGCACAAAGGCAGCAAAGGAG
GAGCGGGACGCCGCCTACGCCGAGGGAAGAATCCCCCTGCTGAGC
CGCGAGGTGGAGGACATCAGGGCAATGCACTCCGTGGTGTGGAGA
GATTCTCTGGGAGCCAGGGCCTTCAGCGGAGGCAAGGCAGAGCAG
TCCCTGGTGTGGCGCGACGAGGAGTTTGGCATCTGGTGCCGGCTG
CGGCCCGATTACGTGCCTAACAATGCCGTGCGGATCTTCGACTAT
AAGACCGCCACAAACGGCTCCCCCGATGCCTTTATGAAGGAGATC
TACAATCGGGGCTATCACCAGCAGGCCGCCTGGTATCTGGACGGA
TATGAGGCAGTGACCGGCCACAGGCCACGCGAGTTCTGGTTTGTG
GTGCAGGAGAAAACCGCCCCCTTCCTGCTGTCTTTCTTTCAGATG
GATGAGATGAGCCTGGAGATCGGCCGGACCCTGAACAGACAGGCC
AAGGGCATCTTTGCCTGGTGCCTGCGCAACAATTGTTGGCCAGGC
TATCAGCCCGAGGTGGATGGCAAGGTGAGATTCTTTACCACATCT
CCCCCTGCCTGGCTGGTGAGGGAGTACGAGTTTAAGAATGAGCAC
GGCGCCTATGAGCCACCCGAGATCAAGCGGAAGGAGGTGGCC
Salmonella enterica subsp. enterica serovar
Javiana str. 10721 RecT DNA
(SEQ ID NO: 107):
CCAAAGCAGCCCCCTATCGCCAAGGCAGACCTGCAGAAAACCCAG
GGAGCACGGACCCCAACAGCAGTGAAGAACAATAACGATGTGATC
TCCTTTATCAATCAGCCTTCTATGAAGGAGCAGCTGGCCGCCGCC
CTGCCAAGGCACATGACCGCCGAGCGGATGATCAGAATCGCCACC
ACAGAGATCAGGAAGGTGCCCGCCCTGGGCGACTGCGATACAATG
TCTTTTGTGAGCGCCATCGTGCAGTGTAGCCAGCTGGGCCTGGAG
CCTGGCGGCGCCCTGGGCCACGCCTACCTGCTGCCTTTCGGCAAT
CGGAACGAGAAGTCCGGCAAGAAGAATGTGCAGCTGATCATCGGC
TATAGAGGCATGATCGACCTGGCCCGGAGATCCGGACAGATCGCC
AGCCTGTCCGCCAGGGTGGTGCGCGAGGGCGACGATTTCTCTTTT
GAGTTCGGCCTGGAGGAGAAGCTGGTGCACAGGCCAGGCGAGAAC
GAGGACGCCCCCGTGACCCACGTGTACGCAGTGGCACGCCTGAAG
GATGGAGGCACCCAGTTTGAAGTGATGACACGGAAGCAGATCGAG
CTGGTGAGAGCCCAGTCTAAGGCCGGCAATAACGGCCCTTGGGTG
ACCCACTGGGAGGAGATGGCCAAGAAAACCGCCATCAGGCGCCTG
TTCAAGTACCTGCCCGTGAGCATCGAGATCCAGAGGGCCGTGAGC
ATGGATGAGAAGGAGACACTGACAATCGACCCAGCCGATGCCAGC
GTGATCACCGGCGAGTATTCCGTGGTGGAGAATGCCGGCGTGGAG
GAGAACGTGACAGCC
Salmonella enterica subsp. enterica serovar
Javiana str. 10721 RecE DNA
(SEQ ID NO: 108):
TACTATGACATCCCAAACGAGGCCTACCACGCAGGCCCCGGCGTG
TCTAAGAGCCAGCTGGACGACATCGCCGATACCCCCGCCATCTAT
CTGTGGCGGAAGAATGCCCCTGTGGACACCGAGAAAACCAAGTCC
CTGGATACCGGCACAGCCTTCCACTGCAGGGTGCTGGAGCCAGAG
GAGTTCAGCAAGCGGTTCATCATCGCCCCCGAGTTCAACCGGAGA
ACCTCCGCCGGCAAGGAGGAGGAGAAAACCTTCCTGGAGGAGTGT
ACCCGGACAGGCAGAACCGTGCTGACAGCCGAGGAGGGCAGGAAG
ATCGAGCTGATGTACCAGTCCGTGATGGCACTGCCACTGGGACAG
TGGCTGGTGGAGTCTGCCGGCTACGCCGAGAGCTCCGTGTATTGG
GAGGACCCTGAGACAGGCATCCTGTGCCGGTGTAGACCCGATAAG
ATCATCCCTGAGTTCCACTGGATCATGGACGTGAAAACCACAGCC
GACATCCAGAGGTTTCGCACCGCCTACTATGACTACAGATACCAC
GTGCAGGACGCCTTCTACTCTGATGGCTATAGAGCCCAGTTTGGC
GAGATCCCTACATTCGTGTTTCTGGTGGCCAGCACCACAGCAGAG
TGCGGCAGATACCCCGTGGAGATCTTTATGATGGGAGAGGACGCA
AAGCTGGCCGGACAGCGCGAGTATAGGCGCAATCTGCAGACCCTG
GCCGAGTGTCTGAACAATGATGAGTGGCCTGCCATCAAGACACTG
TCTCTGCCACGGTGGGCCAAGGAGAACGCCAATGCC
Pseudobacteriovorax antillogorgiicola RecT DNA
(SEQ ID NO: 109):
GGCCACCTGGTGAGCAAGACCGAGCAGGATTACATCAAGCAGCAC
TATGCCAAGGGCGCCACAGACCAGGAGTTCGAGCACTTTATCGGC
GTGTGCAGGGCCAGAGGCCTGAACCCAGCCGCCAATCAGATCTAC
TTCGTGAAGTATCGGTCCAAGGATGGACCAGCAAAGCCAGCCTTT
ATCCTGTCTATCGACAGCCTGAGGCTGATCGCACACCGCACCGGC
GATTACGCAGGATGCTCTGAGCCCATCTTCACAGACGGCGGCAAG
GCCTGTACCGTGACAGTGCGGAGAAACCTGAAGAGCGGCGAGACA
GGCAATTTCTCCGGCATGGCCTTTTATGACGAGCAGGTGCAGCAG
AAGAACGGCCGGCCTACCTCCTTTTGGCAGTCTAAGCCAAGAACA
ATGCTGGAGAAGTGTGCAGAGGCAAAGGCCCTGAGGAAGGCCTTC
CCTCAGGATCTGGGCCAGTTTTACATCAGAGAGGAGATGCCCCCT
CAGTATGACGAGCCTATCCAGGTGCACAAGCCAAAGGCCCTGGAG
GAGCCCAGGTTCAGCAAGTCCGATCTGTCCAGGCGCAAGGGCCTG
AACAGGAAGCTGTCTGCCCTGGGAGTGGACCCCAGCCGCTTCGAT
GAGGTGGCCACCTTTCTGGACGGCACACCTGATCGCGAGCTGGGC
CAGAAGCTGAAGCTGTGGCTGAAGGAGGCCGGCTACGGCGTGAAT
CAG
Pseudobacteriovorax antillogorgiicola RecE DNA
(SEQ ID NO: 110):
AGCAAGCTGTCCAACCTGAAGGTGTCTAATAGCGACGTGGATACA
CTGAGCCGGATCAGAATGAAGGAGGGCGTGTATCGGGACCTGCCA
ATCGAGAGCTACCACCAGTCCCCCGGCTATTCTAAGACCAGCCTG
TGCCAGATCGATAAGGCCCCTATCTACCTGAAAACCAAGGTGCCA
CAGAAGTCCACAAAGTCTCTGAACATCGGCACCGCCTTCCACGAG
GCTATGGAGGGCGTGTTTAAGGACAAGTATGTGGTGCACCCCGAT
CCTGGCGTGAATAAGACCACAAAGTCTTGGAAGGACTTCGTGAAG
AGGTATCCTAAGCACATGCCACTGAAGCGCAGCGAGTACGACCAG
GTGCTGGCCATGTACGATGCCGCCCGGTCTTATAGACCTTTTCAG
AAGTACCACCTGAGCCGGGGCTTCTACGAGAGCTCCTTTTATTGG
CACGATGCCGTGACAAACAGCCTGATCAAGTGCAGACCCGACTAT
ATCACCCCTGATGGCATGAGCGTGATCGACTTCAAGACCACAGTG
GACCCCAGCCCCAAGGGCTTTCAGTACCAGGCCTACAAGTATCAC
TACTACGTGAGCGCCGCCCTGACCCTGGAGGGAATCGAGGCAGTG
ACCGGCATCAGGCCAAAGGAGTACCTGTTCCTGGCCGTGTCCAAT
TCTGCCCCATACCTGACCGCCCTGTATCGCGCCTCTGAGAAGGAG
ATCGCCCTGGGCGACCACTTTATCCGGCGGAGCCTGCTGACCCTG
AAAACCTGTCTGGAGTCTGGCAAGTGGCCCGGCCTGCAGGAGGAG
ATCCTGGAGCTGGGCCTGCCTTTCTCCGGCCTGAAGGAGCTGAGA
GAGGAGCAGGAGGTGGAGGATGAGTTTATGGAGCTGGTGGGC
Photobacterium sp. JCM 19050 RecT DNA
(SEQ ID NO: 111):
AACACCGACATGATCGCCATGCCCCCTTCTCCAGCCATCAGCATG
CTGGACACAAGCAAGCTGGATGTGATGGTGCGGGCAGCAGAGCTG
ATGTCCCAGGCCGTGGTCATGGTGCCCGACCACTTCAAGGGCAAG
CCAGCCGATTGCCTGGCAGTGGTCATGCAGGCAGACCAGTGGGGC
ATGAACCCCTTTACCGTGGCCCAGAAAACCCACCTGGTGAGCGGC
ACCCTGGGATACGAGTCCCAGCTGGTGAATGCCGTGATCAGCTCC
TCTAAGGCCATCAAGGGCCGGTTCCACTATGAGTGGTCTGATGGC
TGGGAGAGACTGGCCGGCAAGGTGCAGTACGTGAAGGAGTCTCGG
CAGAGAAAGGGCCAGCAGGGCAGCTATCAGGTGACCGTGGCCAAG
CCAACATGGAAGCCAGAGGACGAGCAGGGCCTGTGGGTGCGGTGT
GGAGCCGTGCTGGCCGGAGAGAAGGACATCACATGGGGCCCTAAG
CTGTACCTGGCCAGCGTGCTGGTGCGGAACAGCGAGCTGTGGACC
ACAAAGCCCTACCAGCAGGCCGCCTATACCGCCCTGAAGGATTGG
TCCCGCCTGTATACACCTGCCGTGATGCAGGGCTCTATGACCGGC
AAGAGCTGGTCCCTGACAGGCAGGCTGATCAGCCCCCGC
Photobacterium sp. JCM 19050 RecE DNA
(SEQ ID NO: 112):
GCCGAGCGGGTGAGAACCTATCAGCGGGACGCCGTGTTCGCACAC
GAGCTGAAGGCCGAGTTTGATGAGGCCGTGGAGAACGGCAAGACC
GGCGTGACACTGGAGGACCAGGCCAGGGCCAAGAGGATGGTGCAC
GAGGCCACCACAAACCCCGCCTCTCGGAATTGGTTCAGATACGAC
GGAGAGCTGGCCGCATGCGAGAGGAGCTATTTTTGGCGCGATGAG
GAGGCAGGCCTGGTGCTGAAGGCCAGGCCTGACAAGGAGATCGGC
AACAATCTGATCGATGTGAAGTCCATCGAGGTGCCAACCGACGTG
TGCGCCTGTGATCTGAACGCCTATATCAATCGGCAGATCGAGAAG
AGAGGCTACCACATCTCCGCCGCCCACTATCTGTCTGGCACAGGC
AAGGACCGCTTCTTTTGGATCTTCATCAATAAGGTGAAGGGCTAC
GAGTGGGTGGCAATCGTGGAGGCCTCTCCCCTGCACATCGAGCTG
GGCACCTATGAGGTGCTGGAGGGCCTGCGGAGCATCGCCAGCTCC
ACAAAGGAGGCAGATTACCCAGCACCTCTGTCCCACCCTGTGAAC
GAGAGAGGCATCCCACAGCCCCTGATGTCTAATCTGAGCACATAC
GCCATGAAGAGGCTGGAGCAGTTTCGCGAGCTG
Providencia alcalifaciens DSM 30120 RecT DNA
(SEQ ID NO: 113):
AAGGCACAGCTGGCCGCCGCCCTGCCTAAGCACATCACCAGCGAC
CGGATGATCAGAATCGTGTCCACCGAGATCAGAAAGACCCCATCT
CTGGCCAACTGCGACATCCAGAGCTTCATCGGCGCCGTGGTGCAG
TGTTCTCAGCTGGGCCTGGAGCCAGGCAACGCCCTGGGACACGCC
TACCTGCTGCCCTTTGGCAATGGCAAGTCCGACAACGGCAAGTCT
AATGTGCAGCTGATCATCGGCTATCGGGGCATGATCGATCTGGCC
CGGAGAAGCGGCCAGATCATCTCTATCAGCGCCAGGACCGTGCGC
CAGGGCGACAACTTCCACTTTGAGTACGGCCTGAACGAGAATCTG
ACCCACATCCCCGAGGGCAATGAGGACTCCCCTATCACACACGTG
TACGCAGTGGCACGGCTGAAGGATGAGGGCGTGCAGTTCGAAGTG
ATGACATATAACCAGATCGAGAAGGTGAGAGATAGCTCCAAGGCC
GGCAAGAATGGCCCCTGGGTGACCCACTGGGAGGAGATGGCCAAG
AAAACCGTGATCAGGCGCCTGTTTAAGTACCTGCCCGTGAGCATC
GAGATGCAGAAGGCCGTGATCCTGGACGAGAAGGCCGAGGCCAAT
ATCGAGCAGGATCACTCCGCCATCTTCGAGGCCGAGTTTGAGGAG
GTGGACTCTAACGGCAAT
Providencia alcalifaciens DSM 30120 RecE DNA
(SEQ ID NO: 114):
AACGAGGGCATCTACTATGACATCTCTAATGAGGACTATCACCAC
GGCCTGGGCATCTCTAAGAGCCAGCTGGATCTGATCGACGAGAGC
CCCGCCGATTTCATCTGGCACCGGGATGCCCCTGTGGACAACGAG
AAAACCAAGGCCCTGGATTTTGGCACAGCCCTGCACTGCCTGCTG
CTGGAGCCAGACGAGTTCCAGAAGAGGTTTCGCATCGCCCCCGAG
GTGAACCGGAGAACAAATGCCGGCAAGGAGCAGGAGAAGGAGTTC
CTGGAGATGTGCGAGAAGGAGAATATCACCCCCATCACAAACGAG
GATAATAGGAAGCTGTCTCTGATGAAGGACAGCGCAATGGCCCAC
CCTATCGCCCGCTGGTGTCTGGAGGCCAAGGGCATCGCCGAGAGC
TCCATCTATTGGAAGGACAAGGATACAGACATCCTGTGCCGGTGT
AGACCAGACAAGCTGATCGAGGAGCACCACTGGCTGGTGGATGTG
AAGTCCACCGCCGACATCCAGAAGTTCGAGCGGTCTATGTACGAG
TATAGATACCACGTGCAGGATTCCTTTTATTCTGACGGCTACAAG
AGCCTGACAGGCGAGATGCCCGTGTTCGTGTTCCTGGCCGTGTCC
ACCGTGATCAACTGCGGCAGATACCCCGTGCGGGTGTTCGTGCTG
GACGAGCAGGCAAAGTCCGTGGGACGGATCACCTATAAGCAGAAT
CTGTTTACATACGCCGAGTGTCTGAAAACCGACGAGTGGGCCGGC
ATCAGAACCCTGAGCCTGCCCTCCTGGGCAAAGGAGCTGAAGCAC
GAGCACACCACAGCCTCT
Pantoea stewartii RecT Protein
(SEQ ID NO: 115):
MSNQPPIASADLQKANTGKQVANKTPEQTLVGFMNQPAMKSQLAA
ALPRHMTADRMIRIVTTEIRKTPALATCDQSSFIGAVVQCSQLGL
EPGSALGHAYLLPFGNGRSKSGQSNVQLIIGYRGMIDLARRSGQI
VSLSARVVRADDEFSFEYGLDENLIHRPGENEDAPITHVYAVARL
KDGGTQFEVMTVKQIEKVKAQSKASSNGPWVTHWEEMAKKTVIRR
LFKYLPVSIEMQKAVILDEKAESDVDQDNASVLSAEYSVLDGSSE
E
Pantoea stewartii RecE Protein
(SEQ ID NO: 116):
MQPGVYYDISNEEYHAGPGISKSQLDDIAVSPAIFQWRKSAPVDD
EKTAALDLGTALHCLLLEPDEFSKRFMIGPEVNRRTNAGKQKEQD
FLDMCEQQGITPITHDDNRKLRLMRDSAFAHPVARWMLETEGKAE
ASIYWNDRDTQILSRCRPDKLITEFSWCVDVKSTADIGKFQKDFY
SYRYHVQDAFYSDGYEAQFCEVPTFAFLVVSSSIDCGRYPVQVFI
MDQQAKDAGRAEYKRNLTTYAECQARNEWPGIATLSLPYWAKEIR
NV
Pantoea brenneri RecT Protein
(SEQ ID NO: 117):
MSNQPPIASADLQKTQQSKQVANKTPEQTLVGFMNQPAMKSQLAA
ALPRHMTADRMIRIVTTEIRKTPQLAQCDQSSFIGAVVQCSQLGL
EPGSALGHAYLLPFGNGRSKSGQSNVQLIIGYRGMIDLARRSGQI
VSLSARVVRADDEFSFEYGLDENLVHRPGENEDAPITHVYAVARL
KDGGTQFEVMTVKQVEKVKAQSKASSNGPWVTHWEEMAKKTVIRR
LFKYLPVSIEMQKAVVLDEKAESDVDQDNASVLSAEYSVLESGDE
ATN
Pantoea brenneri RecE Protein
(SEQ ID NO: 118):
MQPGIYYDISNEDYHRGAGISKSQLDDIAISPAIYQWRKHAPVDE
EKTAALDLGTALHCLLLEPDEFSKRFQIGPEVNRRTTAGKEKEKE
FIERCEAEGITPITHDDNRKLKLMRDSALAHPIARWMLEAQGNAE
ASIYWNDRDAGVLSRCRPDKIITEFNWCVDVKSTADIMKFQKDFY
SYRYHVQDAFYSDGYESHFHETPTFAFLAVSTSIDCGRYPVQVFI
MDQQAKDAGRAEYKRNIHTFAECLSRNEWPGIATLSLPFWAKELR
NE
Pantoea dispersa RecT Protein
(SEQ ID NO: 119):
MSNQPPLATADLQKTQQSNQVAKTPEQTLVGFMNQPAMKSQLAAA
LPRHMTADRMIRIVTTEIRKTPALAQCDQSSFIGAVVQCSQLGLE
PGSALGHAYLLPFGNGRSKSGQSNVQLIIGYRGMIDLARRSGQIV
SLSARVVRADDEFSFEYGLDENLIHRPGDNESAPITHVYAVARLK
DGGTQFEVMTAKQVEKVKAQSKASSNGPWVTHWEEMAKKTVIRRL
FKYLPVSIEMQKAVVLDEKAESDVDQDNASVLSAEYSVLESGTGE
Pantoea dispersa RecE Protein
(SEQ ID NO: 120):
MEPGIYYDISNEAYHSGPGISKSQLDDIARSPAIFQWRKDAPVDT
EKTKALDLGTDFHCAVLEPERFADMYRVGPEVNRRTTAGKAEEKE
FFEKCEKDGAVPITHDDARKVELMRGSVMAHPIAKQMIAAQGHAE
ASIYWHDESTGNLCRCRPDKFIPDWNWIVDVKTTADMKKFRREFY
DLRYHVQDAFYTDGYAAQFGERPTFVFVVTSTTIDCGRYPTEVFF
LDEETKAAGRSEYQSNLVTYSECLSRNEWPGIATLSLPHWAKELR
NV
Type-F symbiont of Plautia stali RecT Protein
(SEQ ID NO: 121):
MSNQPPIASADLQKTQQSKQVANKTPEQTLVGFMNQPAMKSQLAA
ALPRHMTADRMIRIVTTEIRKTPALATCDQSSFIGAVVQCSQLGL
EPGSALGHAYLLPFGNGRSKSGQSNVQLIIGYRGMIDLARRSGQI
VSLSARVVRADDEFSFEYGLDENLIHRPGDNEDAPITHVYAVARL
KDGGTQFEVMTAKQVEKVKAQSKASSNGPWVTHWEEMAKKTVIRR
LFKYLPVSIEMQKAVVLDEKAESDVDQDNASVLSAEYSVLEGDGG
E
Type-F symbiont of Plautia stali RecE Protein
(SEQ ID NO: 122):
MQPGIYYDISNEDYHGGPGISKSQLDDIAISPAIYQWRKHAPVDE
EKTAALDLGTALHCLLLEPDEFSKRFEIGPEVNRRTTAGKEKEKE
FMERCEAEGVTPITHDDNRKLRLMRDSAMAHPIARWMLEAQGNAE
ASIYWNDRDTGVLSRCRPDKIITDFNWCVDVKSTADIIKFQKDFY
SYRYHVQDAFYSDGYESHFDETPTFAFLAVSTSIDCGRYPVQVFI
MDQQAKDAGRAEYKRNIHTFAECLSRNEWPGIATLSLPYWAKELR
NE
Providencia stuartii RecT Protein
(SEQ ID NO: 123):
MSNPPLAQADLQKTQGTEVKEKTKDQMLVELINKPSMKAQLAAAL
PRHMTPDRMIRIVTTEIRKTPALATCDMQSFVGAVVQCSQLGLEP
GNALGHAYLLPFGNGKSKSGQSNVQLIIGYRGMIDLARRSGQIVS
ISARTVRQGDNFHFEYGLNENLTHVPGENEDSPITHVYAVARLKD
GGVQFEVMTYNQIEKVRASSKAGQNGPWVSHWEEMAKKTVIRRLF
KYLPVSIEMQKAVILDEKAEANIDQENATIFEGEYEEVGTDGK
Providencia stuartii RecE Protein
(SEQ ID NO: 124):
EGIYYNISNEDYHNGLGISKSQLDLINEMPAEYIWSKEAPVDEEK
IKPLEIGTALHCLLLEPDEYHKRYKIGPDVNRRTNAGKEKEKEFF
DMCEKEGITPITHDDNRKLMIMRDSALAHPIAKWCLEADGVSESS
IYWTDKETDVLCRCRPDRIITAHNYIVDVKSSGDIEKFDYEYYNY
RYHVQDAFYSDGYKEVTGITPTFLFLVVSTKIDCGKYPVRTYVMS
EEAKSAGRTAYKHNLLTYAECLKTDEWAGIRTLSLPRWAKELRNE
Providencia sp. MGF014 RecT Protein
(SEQ ID NO: 125):
MSNPPLAQSDLQKTQGTEVKVKTKDQQLIQFINQPSMKAQLAAAL
PRHMTPDRMIRIVTTEIRKTPALATCDMQSFVGAVVQCSQLGLEP
GNALGHAYLLPFGNGKAKSGQSNVQLIIGYRGMIDLARRSNQIIS
ISARTVRQGDNFHFEYGLNEDLTHTPSENEDSPITHVYAVARLKD
GGVQFEVMTYNQVEKVRASSKAGQNGPWVSHWEEMAKKTVIRRLF
KYLPVSIEMQKAVVLDEKAEANVDQENATIFEGEYEEVGTDGN
Providencia sp. MGF014 RecE Protein
(SEQ ID NO: 126):
MKEGIYYNISNEDYHNGLGISKSQLDLINEMPAEYIWSKEAPVDE
EKIKPLEIGTALHCLLLEPDEYHKRYKIGPDVNRRTNVGKEKEKE
FFDMCEKEGITPITHDDNRKLMIMRDSALAHPIAKWCLEADGVSE
SSIYWTDKETDVLCRCRPDRIITAHNYIIDVKSSGDIEKFDYEYY
NYRYHVQDAFYSDGYKEVTGITPTFLFLVVSTKIDCGKYPVRTYV
MSEEAKSAGRTAYKHNLLTYAECLKTDEWAGIRTLSLPRWAKELR
NE
Shewanella putrefaciens RecT Protein
(SEQ ID NO: 127):
MQTAQVKLSVPHQQVYQDNFNYLSSQVVGHLVDLNEEIGYLNQIV
ENSLSTASPLDVAAPWSVYGLLLNVCRLGLSLNPEKKLAYVMPSW
SETGEIIMKLYPGYRGEIAIASNENVIKNANAVLVYENDHFRIQA
ATGEIEHFVTSLSIDPRVRGACSGGYCRSVLMDNTIQISYLSIEE
MNAIAQNQIEANMGNTPWNSIWRTEMNRVALYRRAAKDWRQLIKA
TPEIQSALSDTEY
Shewanella putrefaciens RecE Protein
(SEQ ID NO: 128):
MGTALAQTISLDWQDTIQPAYTASGKPNFLNAQGEIVEGIYTDLP
NSVYHALDAHSSTGIKTFAKGRHHYFRQYLSDVCRQRTKQQEYTF
DAGTYGHMLVLEPENFHGNFMRNPVPDDFPDIELIESIPQLKAAL
AKSNLPVSGAKAALIERLYAFDPSLPLFEKMREKAITDYLDLRYA
KYLRTDVELDEMATFYGIDTSQTREKKIEEILAISPSQPIWEKLI
SQHVIDHIVWDDAMRVERSTRAHPKADWLISDGYAELTIIARCPT
TGLLLKVRFDWLRNDAIGVDFKTTLSTNPTKFGYQIKDLRYDLQQ
VFYCYVANLAGIPVKHFCFVATEYKDADNCETFELSHKKVIESTE
EMFDLLDEFKEALTSGNWYGHDRSRSTWVIEV
Bacillus sp. MUM 116 RecT Protein
(SEQ ID NO: 129):
MSKQLTTVNTQAVVGTFSQAELDTLKQTIAKGTTNEQFALFVQTC
ANSRLNPFLNHIHCIVYNGKEGATMSLQIAVEGILYLARKTDGYK
GIECQLIHENDEFKFDAKSKEVDHQIGFPRGNVIGGYAIAKREGF
DDVVVLMESNEVDHMLKGRNGHMWRDWFNDMFKKHIMKRAAKLQY
GIEIAEDETVSSGPSVDNIPEYKPQPRKDITPNQDVIDAPPQQPK
QDDEAAKLKAARSEVSKKFKKLGIVKEDQTEYVEKHVPGFKGTLS
DFIGLSQLLDLNIEAQEAQSADGDLLD
Bacillus sp. MUM 116 RecE Protein
(SEQ ID NO: 130):
MTYAADETLVQLLLSVDGKQLLLGRGLKKGKAQYYINEVPSKAKE
FEEIRDQLFDKDLFMSLFNPSYFFTLHWEKQRAMMLKYVTAPVSK
EVLKNLPEAQSEVLERYLKKHSLVDLEKIHKDNKNKQDKAYISAQ
SRTNTLKEQLMQLTEEKLDIDSIKAELAHIDMQVIELEKQMDTAF
EKNQAFNLQAQIRNLQDKIEMSKERWPSLKNEVIEDTCRTCKRPL
DEDSVEAVKADKDNRIAEYKAKHNSLVSQRNELKEQLNTIEYIDV
TELREQIKELDESGQPLREQVRIYSQYQNLDTQVKSAEADENGIL
QDLKASIFILDSIKAFRGKEAEMQAEKVQALFTTLSVRLFKQNKG
DGEIKPDFEIEMNDKPYRTLSLSEGIRAGLELRDVLSQQSELVTP
TFVDNAESITSFKQPNGQLIISRVVAGQELKIEAVSE
Shigella sonnei RecT Protein
(SEQ ID NO: 131):
MTKQPPIAKADLQKTQENRAPAAIKNNDVISFINQPSMKEQLAAA
LPRHMTAERMIRIATTEIRKVPALGNCDTMSFVSAIVQCSQLGLE
PGSALGHAYLLPFGNKNEKSGKKNVQLIIGYRGMIDLARRSGQIA
SLSARVVREGDEFNFEFGLDEKLIHRPGENEDAPVTHVYAVARLK
DGGTQFEVMTRRQIELVRSQSKAGNNGPWVTHWEEMAKKTAIRRL
FKYLPVSIEIQRAVSMDEKEPLTIDPADSSVLTGEYSVIDNSEE
Shigella sonnei RecE Protein
(SEQ ID NO: 132):
DRGLLTKEWRKGNRVSRITRTASGANAGGGNLTDRGEGFVHDLTS
LARDIATGVLARSMDVDIYNLHPAHAKRIEEIIAENKPPFSVFRD
KFITMPGGLDYSRAIVVASVKEAPIGIEVIPAHVTAYLNKVLTET
DHANPDPEIVDIACGRSSAPMPQRVTEEGKQDDEEKLQPSGTTAD
EQGEAETMEPDATKHHQDTQPLDAQSQVNSVDAKYQELRAELHEA
RKNIPSKNPVDADKLLAASRGEFVDGISDPNDPKWVKGIQTRDSV
YQNQPETEKTSPDMKQPEPVVQQEPEIAFNACGQTGGDNCPDCGA
VMGDATYQETFDEENQVEAKENDPEEMEGAEHPHNENAGSDPHRD
CSDETGEVADPVIVEDIEPGIYYGISNENYHAGPGVSKSQLDDIA
DTPALYLWRKNAPVDTTKTKTLDLGTAFHCRVLEPEEFSNRFIVA
PEFNRRTNAGKEEEKAFLMECASTGKMVITAEEGRKIELMYQSVM
ALPLGQWLVESAGHAESSIYWEDPETGILCRCRPDKIIPEFHWIM
DVKTTADIQRFKTAYYDYRYHVQDAFYSDGYEAQFGVQPTFVFLV
ASTTIECGRYPVEIFMMGEEAKLAGQLEYHRNLRTLADCLNTDEW
PAIKTLSLPRWAKEYAND
Salmonella enterica RecT Protein
(SEQ ID NO: 133):
MTKQPPIAKADLQKTQGNRAPAAVNDKDVLCVINSPAMKAQLAAA
LPRHMTAERMIRIATTEIRKVPELRNCDSTSFIGAIVQCSQLGLE
PGSALGHAYLLPFGNGKAKNGKKNVQLIIGYRGMIDLARRSGQII
SLSARVVRECDEFSYELGLDEKLVHRPGENEDAPITHVYAVAKLK
DGGVQFEVMTKKQVEKVRDTHSKAAKNAASKGASSIWDEHFEDMA
KKTVIRKLFKYLPVSIEIQRAVSMDGKEVETINPDDISVIAGEYS
VIDNPEE
Salmonella enterica RecE Protein
(SEQ ID NO: 134):
DRGLLTKEWRKGNRVSRITRTASGANAGGGNLTDRGEGFVHDLTS
LARDVATGVLARSMDVDIYNLHPAHAKRVEEIIAENKPPFSVFRD
KFITMPGGLDYSRAIVVASVKEAPIGIEVIPAHVTEYLNKVLTET
DHANPDPEIVDIACGRSSAPMPQRVTEEGKQDDEEKPQPSGAMAD
EQATAETVEPNATEHHQNTQPLDAQSQVNSVDAKYQELRAELQEA
RKNIPSKNPVDADKLLAASRGEFVDGISDPNDPKWVKGIQTRDSV
YQNQPETEKISPDAKQPEPVVQQEPETVCNACGQTGGDNCPDCGA
VMGDATYQETFGEENQVEAKEKDPEEMEGAEHPHNENAGSDPHRD
CSDETGEVADPVIVEDIEPGIYYGISNENYHAGPGVSKSQLDDIA
DTPALYLWRKNAPVDTTKTKTLDLGTAFHCRVLEPEEFSNRFIVA
PEFNRRTNAGKEEEKAFLMECASTGKTVITAEEGRKIELMYQSVM
ALPLGQWLVESAGHAESSIYWEDPETGILCRCRPDKIIPEFHWIM
DVKTTADIQRFKTAYYDYRYHVQDAFYSDGYEAQFGVQPTFVFLV
ASTTVECGRYPVEIFMMGEEAKLAGQQEYHRNLRTLADCLNTDEW
PAIKTLSLPRWAKEYAND
Acetobacter RecT Protein
(SEQ ID NO: 135):
MNAPQKQNTRAAVKKISPQEFAEQFAAIIPQVKSVLPAHVTFEKF
ERVVRLAVRKNPDLLTCSPASLFMACIQAASDGLLPDGREGAIVS
RWSSKKSCNEASWMPMVAGLMKLARNSGDIASISSQVVFEGEHFR
VVLGDEERIEHERDLGKTGGKIVAAYAVARLKDGSDPIREIMSWG
QIEKIRNTNKKWEWGPWKAWEDEMARKTVIRRLAKRLPMSTDKEG
ERLRSAIERIDSLVDISANVDAPQIAADDEFAAAAHGVEPQQIAA
PDLIGRLAQMQSLEQVQDIEPQVSHAIQEADKRGDSDTANALDAA
LQSALSRTSTAKEEVPA
Acetobacter RecE Protein
(SEQ ID NO: 136):
MVISKSGIYDLTNEQYHADPCPEMSLSSSGARDLLSSCPAKFIAA
KQLPQQNKRCFDIGSAGHLMVLEPHLFDQKVCEIKHPDWRTKAAK
EERDAAYAEGRIPLLSREVEDIRAMHSVVWRDSLGARAFSGGKAE
QSLVWRDEEFGIWCRLRPDYVPNNAVRIFDYKTATNGSPDAFMKE
IYNRGYHQQAAWYLDGYEAVTGHRPREFWFVVQEKTAPFLLSFFQ
MDEMSLEIGRTLNRQAKGIFAWCLRNNCWPGYQPEVDGKVRFFTT
SPPAWLVREYEFKNEHGAYEPPEIKRKEVA
Salmonella enterica subsp. enterica serovar
Javiana str. 10721 RecT Protein
(SEQ ID NO: 137):
MPKQPPIAKADLQKTQGARTPTAVKNNNDVISFINQPSMKEQLAA
ALPRHMTAERMIRIATTEIRKVPALGDCDTMSFVSAIVQCSQLGL
EPGGALGHAYLLPFGNRNEKSGKKNVQLIIGYRGMIDLARRSGQI
ASLSARVVREGDDFSFEFGLEEKLVHRPGENEDAPVTHVYAVARL
KDGGTQFEVMTRKQIELVRAQSKAGNNGPWVTHWEEMAKKTAIRR
LFKYLPVSIEIQRAVSMDEKETLTIDPADASVITGEYSVVENAGV
EENVTA
Salmonella enterica subsp. enterica serovar
Javiana str. 10721 RecE Protein
(SEQ ID NO: 138):
MYYDIPNEAYHAGPGVSKSQLDDIADTPAIYLWRKNAPVDTEKTK
SLDTGTAFHCRVLEPEEFSKRFIIAPEFNRRTSAGKEEEKTFLEE
CTRTGRTVLTAEEGRKIELMYQSVMALPLGQWLVESAGYAESSVY
WEDPETGILCRCRPDKIIPEFHWIMDVKTTADIQRFRTAYYDYRY
HVQDAFYSDGYRAQFGEIPTFVFLVASTTAECGRYPVEIFMMGED
AKLAGQREYRRNLQTLAECLNNDEWPAIKTLSLPRWAKENANA
Pseudobacteriovorax antillogorgiicola RecT
Protein
(SEQ ID NO: 139):
MGHLVSKTEQDYIKQHYAKGATDQEFEHFIGVCRARGLNPAANQI
YFVKYRSKDGPAKPAFILSIDSLRLIAHRTGDYAGCSEPIFTDGG
KACTVTVRRNLKSGETGNFSGMAFYDEQVQQKNGRPTSFWQSKPR
TMLEKCAEAKALRKAFPQDLGQFYIREEMPPQYDEPIQVHKPKAL
EEPRFSKSDLSRRKGLNRKLSALGVDPSRFDEVATFLDGTPDREL
GQKLKLWLKEAGYGVNQ
Pseudobacteriovorax antillogorgiicola RecE
Protein
(SEQ ID NO: 140):
MSKLSNLKVSNSDVDTLSRIRMKEGVYRDLPIESYHQSPGYSKTS
LCQIDKAPIYLKTKVPQKSTKSLNIGTAFHEAMEGVFKDKYVVHP
DPGVNKTTKSWKDFVKRYPKHMPLKRSEYDQVLAMYDAARSYRPF
QKYHLSRGFYESSFYWHDAVTNSLIKCRPDYITPDGMSVIDFKTT
VDPSPKGFQYQAYKYHYYVSAALTLEGIEAVTGIRPKEYLFLAVS
NSAPYLTALYRASEKEIALGDHFIRRSLLTLKTCLESGKWPGLQE
EILELGLPFSGLKELREEQEVEDEFMELVG
Photobacterium sp. JCM 19050 RecT Protein
(SEQ ID NO: 141):
MNTDMIAMPPSPAISMLDTSKLDVMVRAAELMSQAVVMVPDHFKG
KPADCLAVVMQADQWGMNPFTVAQKTHLVSGTLGYESQLVNAVIS
SSKAIKGRFHYEWSDGWERLAGKVQYVKESRQRKGQQGSYQVTVA
KPTWKPEDEQGLWVRCGAVLAGEKDITWGPKLYLASVLVRNSELW
TTKPYQQAAYTALKDWSRLYTPAVMQGSMTGKSWSLTGRLISPR
Photobacterium sp. JCM 19050 RecE Protein
(SEQ ID NO: 142):
MAERVRTYQRDAVFAHELKAEFDEAVENGKTGVTLEDQARAKRMV
HEATTNPASRNWFRYDGELAACERSYFWRDEEAGLVLKARPDKEI
GNNLIDVKSIEVPTDVCACDLNAYINRQIEKRGYHISAAHYLSGT
GKDRFFWIFINKVKGYEWVAIVEASPLHIELGTYEVLEGLRSIAS
STKEADYPAPLSHPVNERGIPQPLMSNLSTYAMKRLEQFREL
Providencia alcalifaciens DSM 30120 RecT Protein
(SEQ ID NO: 143):
MKAQLAAALPKHITSDRMIRIVSTEIRKTPSLANCDIQSFIGAVV
QCSQLGLEPGNALGHAYLLPFGNGKSDNGKSNVQLIIGYRGMIDL
ARRSGQIISISARTVRQGDNFHFEYGLNENLTHIPEGNEDSPITH
VYAVARLKDEGVQFEVMTYNQIEKVRDSSKAGKNGPWVTHWEEMA
KKTVIRRLFKYLPVSIEMQKAVILDEKAEANIEQDHSAIFEAEFE
EVDSNGN
Providencia alcalifaciens DSM 30120 RecE Protein
(SEQ ID NO: 144):
MNEGIYYDISNEDYHHGLGISKSQLDLIDESPADFIWHRDAPVDN
EKTKALDFGTALHCLLLEPDEFQKRFRIAPEVNRRTNAGKEQEKE
FLEMCEKENITPITNEDNRKLSLMKDSAMAHPIARWCLEAKGIAE
SSIYWKDKDTDILCRCRPDKLIEEHHWLVDVKSTADIQKFERSMY
EYRYHVQDSFYSDGYKSLTGEMPVFVFLAVSTVINCGRYPVRVFV
LDEQAKSVGRITYKQNLFTYAECLKTDEWAGIRTLSLPSWAKELK
HEHTTAS
Mouse Albumin knock-in sense template
(SEQ ID NO: 160)
CACCTTCAGATTTTCCTGTAACGATCGGGAACTGGCATCTTCAGG
GAGTAGctgacctcttctcttcctcccacaggATCCTGGAGCCAC
CCGCAGTTCGAAAAGCTCAGTGAAGAGAAGAACAAAAAGCAGCAT
ATTACAGTTAGTTGTCTTCATCAATCTTTAAATATGTTGTGTGGT
TTTTCTCTCCCTGTTTCCAC
Mouse Albumin knock-in anti-sense template
(SEQ ID NO: 161)
GTGGAAACAGGGAGAGAAAAACCACACAACATATTTAAAGATTGA
TGAAGACAACTAACTGTAATATGCTGCTTTTTGTTCTTCTCTTCA
CTGAGCTTTTCGAACTGCGGGTGGCTCCAGGATcctgtgggagga
agagaagaggtcagCTACTCCCTGAAGATGCCAGTTCCCGATCGT
TACAGGAAAATCTGAAGGTG
(SEQ ID NO: 162)
ACTTTGAGTGTAGCAGAGAGGAACCATTGCCACCTTCAGATTTTC
CTGTAACGATCGGGAACTGGCATCTTCAGGGAGTAGCTGACCTCT
TCTCTTCCTCCCACAGGATCCTGGAGCCACC

### Example 16

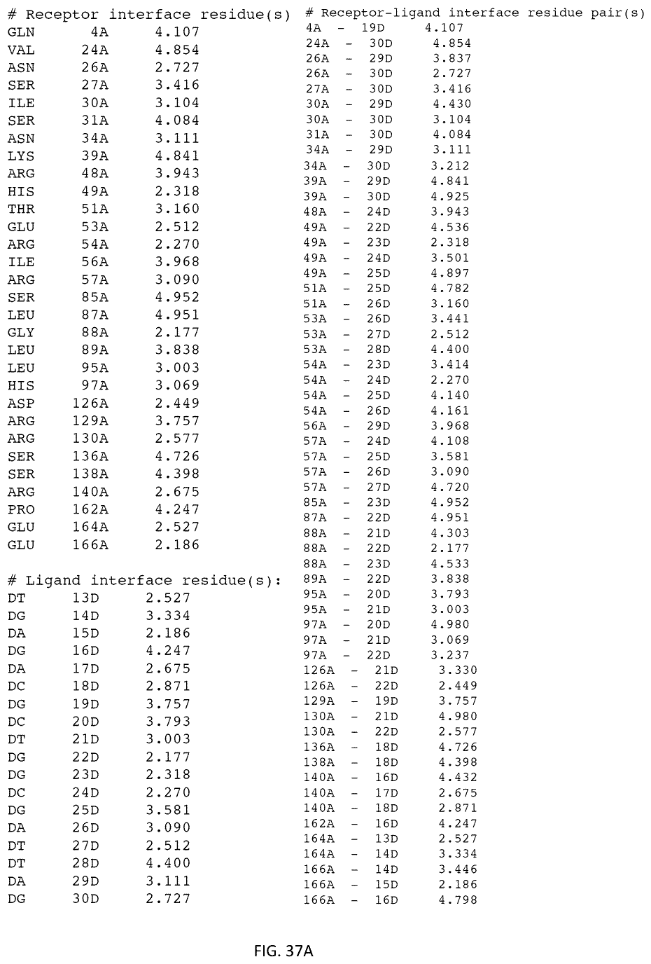

The structure of E. coli RecT (EcRecT) alone (FIG. 36A) and with bound single-strand DNA (FIGS. 36B and 36C) was predicted. The contact interface is consistent with truncation data (Example 7, FIG. 20A). Predicted interactions of EcRecT SSAP amino acids with DNA are shown in FIGS. 37A and 37B.

### Example 17

322 new SSAP proteins were identified from sequence data, synthesized, and screened for activity with Cas9 and dCas9. Gene editing activities are shown below in Table 5, followed by amino acid sequences of the proteins.

TABLE 5
Cas9 Activity with SSAP

| | | | Cas9 with | | dCas9 with |
|---|---|---|---|---|---|
| SSAP | Activity | Cas9 with | SSAP | dCas9 with | SSAP |
| Protein ID | across all | SSAP | activity | SSAP | activity |
| (Uniprot) | experiments | activity | (normalized) | activity | (normalized) |
| WP_130123223.1 | 2.999 | 2.358 | 1.579 | 3.640 | 2.062 |
| SEI77195.1 | 1.811 | 1.340 | 0.516 | 2.282 | 1.278 |
| WP_038246219.1 | 0.121 | −1.654 | −0.724 | 1.896 | 2.101 |
| WP_149216302.1 | 0.692 | −0.279 | −0.275 | 1.664 | 1.531 |
| UPI000B4BEFE6 | −0.284 | −1.881 | −0.590 | 1.313 | 1.563 |
| UPI0000030D3A | 0.058 | −0.942 | −0.788 | 1.058 | 0.695 |
| WP_074846740.1 | 0.558 | 0.254 | 0.626 | 0.863 | 0.407 |
| UPI0008EA8633 | 0.551 | 0.397 | 0.895 | 0.705 | 1.131 |
| UPI000958E115 | 0.781 | 0.893 | 0.751 | 0.668 | 0.984 |
| CDA71469.1 | −1.027 | −2.602 | −3.388 | 0.549 | 0.332 |
| UPI000795D815 | 0.534 | 0.757 | 0.560 | 0.311 | 0.109 |
| WP_069728515.1 | 0.393 | 0.562 | 0.533 | 0.225 | 0.240 |
| UPI00064B44C1 | −0.190 | −0.544 | −0.573 | 0.163 | 0.148 |
| UPI00064D5E13 | −0.778 | −1.644 | −1.327 | 0.088 | 0.036 |
| RTL04618.1 | 0.184 | 0.287 | 0.247 | 0.082 | 0.048 |
| WP_037404193.1 | −0.857 | −1.623 | −0.850 | −0.092 | −0.051 |
| OIO76374.1 | −0.501 | −0.905 | −0.436 | −0.097 | −0.073 |
| GAK01483.1 | −0.236 | −0.293 | −0.508 | −0.179 | −0.069 |
| UPI0007F13B78 | 0.082 | 0.370 | 0.407 | −0.205 | −0.040 |
| WP_106316803.1 | −1.424 | −2.628 | −0.424 | −0.220 | −0.063 |
| WP_102086779.1 | 0.114 | 0.635 | 0.826 | −0.407 | −0.170 |
| WP_114599505.1 | −0.120 | 0.257 | 0.239 | −0.498 | −0.164 |
| UPI000034E66D | −1.223 | −1.791 | −0.870 | −0.655 | −0.866 |
| UPI0008E12231 | −1.249 | −1.797 | −2.283 | −0.701 | −0.555 |
| UPI00015968D7 | −1.288 | −1.876 | −0.850 | −0.701 | −2.250 |
| UPI0008D18539 | −3.014 | −5.286 | −0.810 | −0.742 | −0.240 |
| WP_125711747.1 | −0.150 | 0.465 | 0.410 | −0.764 | −0.279 |
| WP_129141488.1 | −0.451 | −0.118 | −0.065 | −0.784 | −0.325 |
| UPI0001E0C499 | 0.498 | 1.789 | 4.337 | −0.792 | −0.867 |
| WP_135329961.1 | −0.932 | −1.057 | −0.929 | −0.807 | −0.626 |
| UPI000009AF52 | −0.440 | −0.026 | −0.032 | −0.854 | −0.392 |
| GAE17732.1 | −0.575 | −0.275 | −0.560 | −0.876 | −0.635 |
| WP_061413958.1 | −0.371 | 0.154 | 0.174 | −0.896 | −0.293 |
| UPI0005E4CB74 | −0.751 | −0.515 | −0.176 | −0.987 | −0.289 |
| WP_118016648.1 | −1.214 | −1.429 | −0.887 | −1.000 | −0.320 |
| WP_071062796.1 | −1.168 | −1.284 | −1.104 | −1.053 | −0.424 |
| UPI00015C02E0 | −0.687 | −0.287 | −0.195 | −1.088 | −0.491 |
| WP_017415747.1 | −0.758 | −0.428 | −0.545 | −1.088 | −1.120 |
| WP_084505057.1 | −1.095 | −1.090 | −2.185 | −1.100 | −0.907 |
| WP_079708113.1 | −0.030 | 1.070 | 2.231 | −1.129 | −1.000 |
| WP_125141636.1 | 0.144 | 1.429 | 0.558 | −1.142 | −0.148 |
| WP_069686512.1 | −0.656 | −0.160 | −0.383 | −1.151 | −0.400 |
| CCZ61365.1 | 0.233 | 1.622 | 2.756 | −1.156 | −0.358 |
| WP_117624242.1 | −1.613 | −2.047 | −0.694 | −1.179 | −0.244 |
| WP_130067396.1 | −1.050 | −0.911 | −3.635 | −1.189 | −0.361 |
| UPI00078ED021 | −0.491 | 0.218 | 0.538 | −1.201 | −0.336 |
| WP_016998679.1 | −2.985 | −4.713 | −0.683 | −1.257 | −0.422 |
| WP_002566991.1 | −2.868 | −4.474 | −1.192 | −1.263 | −0.179 |
| AGF93134.1 | −0.985 | −0.693 | −1.286 | −1.277 | −1.011 |
| UPI0001BEF484 | −0.847 | −0.298 | −0.329 | −1.396 | −0.389 |
| WP_018705791.1 | −0.622 | 0.173 | 0.129 | −1.417 | −0.419 |
| WP_109615067.1 | 0.412 | 2.341 | 2.795 | −1.516 | −0.177 |
| WP_019168122.1 | −1.867 | −2.201 | −0.296 | −1.534 | −0.179 |
| UPI0009805C1D | −0.687 | 0.284 | 0.225 | −1.658 | −0.897 |
| WP_082209600.1 | −0.753 | 0.244 | 0.617 | −1.749 | −0.598 |
| WP_052399147.1 | −1.321 | −0.840 | −0.758 | −1.801 | −0.508 |
| SFO83314.1 | −1.554 | −1.283 | −0.722 | −1.826 | −2.544 |
| WP_107378794.1 | −1.198 | −0.553 | −1.314 | −1.842 | −1.900 |
| WP_141925904.1 | −0.846 | 0.236 | 0.173 | −1.928 | −0.948 |
| WP_077867213.1 | −1.394 | −0.842 | −0.289 | −1.946 | −0.772 |
| WP_124537594.1 | −0.650 | 0.714 | 1.299 | −2.015 | −0.557 |
| WP_138600901.1 | −2.188 | −2.337 | −0.343 | −2.038 | −0.267 |
| AAT90028.1 | −0.980 | 0.085 | 0.091 | −2.044 | −1.493 |
| WP_068672306.1 | −2.301 | −2.551 | −0.452 | −2.052 | −0.337 |
| WP_051267408.1 | −1.635 | −1.201 | −0.666 | −2.069 | −0.685 |
| WP_027347470.1 | −0.900 | 0.273 | 0.278 | −2.073 | −1.380 |
| WP_096823857.1 | −2.122 | −2.139 | −0.908 | −2.105 | −0.826 |
| WP_136710836.1 | 0.449 | 3.010 | 2.232 | −2.113 | −0.351 |
| SYW13692.1 | −2.400 | −2.670 | −0.476 | −2.129 | −0.311 |
| WP_118771779.1 | −0.974 | 0.197 | 0.073 | −2.145 | −1.216 |
| WP_027295741.1 | −1.538 | −0.918 | −0.925 | −2.157 | −0.568 |
| UPI0002B78771 | −1.308 | −0.435 | −1.203 | −2.180 | −0.281 |
| UPI000865FB15 | −0.560 | 1.110 | 4.696 | −2.230 | −0.296 |
| WP_106478153.1 | −2.036 | −1.798 | −0.307 | −2.273 | −0.413 |
| UPI000B36BD3F | −2.308 | −2.257 | −0.341 | −2.360 | −0.367 |
| WP_106064284.1 | −1.072 | 0.280 | 0.259 | −2.423 | −0.328 |
| WP_024292388.1 | −1.200 | 0.031 | 0.019 | −2.431 | −0.313 |
| CDF42377.1 | −1.325 | −0.211 | −0.214 | −2.440 | −0.309 |
| BAQ93806.1 | −4.877 | −7.274 | −1.400 | −2.480 | −0.364 |
| WP_081955873.1 | −0.883 | 0.742 | 1.773 | −2.508 | −0.276 |
| UPI00065C2D47 | −1.264 | −0.011 | −0.061 | −2.517 | −0.565 |
| WP_147981944.1 | −3.320 | −4.061 | −0.571 | −2.578 | −0.849 |
| WP_028113352.1 | −1.240 | 0.114 | 0.106 | −2.593 | −0.657 |
| WP_045553720.1 | −1.217 | 0.216 | 0.821 | −2.649 | −1.189 |
| UPI00025CF49A | −0.927 | 0.813 | 0.564 | −2.667 | −0.795 |
| WP_020007369.1 | −3.455 | −4.226 | −0.744 | −2.685 | −0.421 |
| WP_016979878.1 | −0.971 | 0.754 | 0.207 | −2.696 | −0.444 |
| WP_148820236.1 | −1.758 | −0.806 | −0.733 | −2.710 | −0.380 |
| LambdaBet | −3.613 | −4.490 | −0.856 | −2.737 | −0.416 |
| UPI0007B64693 | −0.583 | 1.588 | 1.090 | −2.755 | −1.023 |
| WP_143887802.1 | −4.836 | −6.903 | −1.680 | −2.770 | −0.443 |
| UPI0001E2AFC1 | −0.998 | 0.839 | 0.359 | −2.835 | −0.450 |
| WP_072526012.1 | −0.742 | 1.362 | 1.914 | −2.847 | −0.374 |
| UPI00062002D2 | −1.512 | −0.124 | −0.406 | −2.900 | −0.907 |
| WP_060905391.1 | −1.873 | −0.836 | −0.308 | −2.910 | −0.482 |
| WP_013655830.1 | −1.177 | 0.596 | 1.140 | −2.950 | −0.446 |
| WP_131521405.1 | −1.882 | −0.802 | −0.382 | −2.961 | −0.450 |
| WP_100916003.1 | −2.321 | −1.578 | −0.629 | −3.063 | −2.608 |
| RDC50983.1 | −3.431 | −3.728 | −3.241 | −3.134 | −3.818 |
| WP_063601171.1 | −2.057 | −0.932 | −0.510 | −3.182 | −0.478 |
| AFH22576.1 | −3.156 | −3.033 | −0.543 | −3.279 | −0.575 |
| WP_064664300.1 | −2.305 | −1.322 | −0.932 | −3.288 | −0.449 |
| WP_002595146.1 | −5.320 | −7.323 | −1.345 | −3.318 | −0.536 |
| WP_109401438.1 | −1.672 | −0.020 | −0.011 | −3.325 | −0.539 |
| UPI000212F382 | −1.354 | 0.692 | 1.723 | −3.399 | −0.852 |
| UPI00078E90BE | −3.391 | −3.376 | −0.549 | −3.405 | −0.557 |
| WP_115149784.1 | −1.869 | −0.322 | −1.581 | −3.416 | −1.995 |
| KKZ74881.1 | −2.669 | −1.842 | −1.968 | −3.497 | −0.528 |
| WP_089281299.1 | −2.336 | −1.166 | −0.249 | −3.506 | −0.681 |
| GAC42786.1 | −5.493 | −7.473 | | −3.513 | −0.512 |
| WP_099840029.1 | −1.897 | −0.224 | −0.145 | −3.571 | −0.553 |
| WP_132110073.1 | −2.204 | −0.830 | −0.252 | −3.577 | −0.602 |
| WP_100306418.1 | −4.276 | −4.946 | −1.095 | −3.605 | −0.658 |
| UPI0001D2DF22 | −2.192 | −0.774 | −0.320 | −3.610 | −0.537 |
| UPI0000010203 | −4.270 | −4.919 | −0.814 | −3.622 | −0.496 |
| WP_066790810.1 | −1.807 | 0.069 | 0.068 | −3.682 | −0.539 |
| WP_068720576.1 | −0.901 | 1.888 | 0.942 | −3.690 | −0.763 |
| WP_123849158.1 | −2.441 | −1.178 | −0.366 | −3.704 | −0.608 |
| WP_115407185.1 | −1.849 | 0.045 | 0.060 | −3.743 | −0.597 |
| WP_083048409.1 | −3.591 | −3.406 | −0.596 | −3.777 | −0.714 |
| WP_109523733.1 | −1.640 | 0.510 | 0.610 | −3.790 | −0.578 |
| WP_106833617.1 | −4.559 | −5.271 | −0.961 | −3.846 | −0.563 |
| WP_019417330.1 | −1.537 | 0.824 | 0.919 | −3.897 | −0.611 |
| OAB27843.1 | −1.809 | 0.282 | 0.572 | −3.899 | −0.575 |
| WP_045958294.1 | −2.219 | −0.504 | −0.093 | −3.935 | −0.892 |
| WP_147129628.1 | −1.788 | 0.372 | 0.623 | −3.947 | −0.622 |
| UPI000B94B1D1 | −1.700 | 0.575 | 0.280 | −3.974 | −0.714 |
| WP_092601202.1 | −0.786 | 2.444 | 1.806 | −4.016 | −0.671 |
| WP_115856892.1 | −1.672 | 0.679 | 1.062 | −4.023 | −0.498 |
| UPI0008D990CB | −2.666 | −1.263 | −0.349 | −4.070 | −0.652 |
| UPI0002AD92E7 | −4.468 | −4.826 | −0.842 | −4.110 | −0.643 |
| UPI00020BA2E0 | −1.758 | 0.597 | 0.880 | −4.113 | −0.660 |
| SDL28883.1 | −2.528 | −0.924 | −0.410 | −4.132 | −0.827 |
| CDE68291.1 | −2.313 | −0.476 | −0.351 | −4.151 | −0.615 |
| WP_120191052.1 | −1.782 | 0.596 | 1.792 | −4.159 | −0.615 |
| WP_073793143.1 | −1.792 | 0.580 | 0.924 | −4.163 | −0.726 |
| WP_098408280.1 | −2.932 | −1.697 | −0.475 | −4.168 | −0.640 |
| WP_099299656.1 | −2.669 | −1.160 | −0.694 | −4.178 | −0.675 |
| UPI0001CE597A | −2.181 | −0.183 | −0.399 | −4.179 | −0.615 |
| WP_125769509.1 | −3.017 | −1.843 | −1.377 | −4.191 | −0.610 |
| GAE09585.1 | −4.441 | −4.631 | −0.809 | −4.250 | −0.686 |
| UPI0009805F63 | −2.282 | −0.308 | −0.267 | −4.256 | −0.591 |
| WP_131535536.1 | −2.345 | −0.432 | −0.643 | −4.259 | −1.301 |
| UPI0001594E53 | −2.297 | −0.331 | −0.351 | −4.263 | −0.702 |
| WP_138067957.1 | −4.402 | −4.531 | −1.341 | −4.273 | −1.117 |
| UPI0005FEB4B0 | −1.644 | 0.998 | 0.451 | −4.286 | −0.712 |
| WP_092453396.1 | −3.266 | −2.221 | −0.998 | −4.310 | −0.557 |
| UPI00078EBE91 | −3.018 | −1.710 | −0.437 | −4.326 | −0.647 |
| UPI00091F1EB0 | −2.055 | 0.274 | 0.640 | −4.383 | −0.616 |
| WP_067592792.1 | −2.958 | −1.480 | −0.437 | −4.435 | −0.734 |
| PCR98661.1 | −2.757 | −1.056 | −0.259 | −4.457 | −0.721 |
| RDI65706.1 | −4.494 | −4.531 | −1.341 | −4.458 | −1.272 |
| UPI000B38B374 | −4.391 | −4.284 | −0.776 | −4.498 | −0.873 |
| WP_084261900.1 | −2.611 | −0.682 | −0.190 | −4.540 | −0.705 |
| UPI0007BCAEAB | −2.185 | 0.198 | 0.190 | −4.569 | −0.746 |
| WP_132769795.1 | −5.073 | −5.574 | −1.211 | −4.572 | −0.829 |
| WP_028562280.1 | −2.900 | −1.206 | −0.494 | −4.593 | −0.870 |
| WP_034910107.1 | −5.290 | −5.948 | −1.267 | −4.633 | −0.842 |
| WP_009524931.1 | −2.714 | −0.778 | −0.800 | −4.650 | −0.774 |
| T7gp2.5 | −2.001 | 0.660 | 1.440 | −4.662 | −0.557 |
| WP_118206945.1 | −5.345 | −6.013 | −1.700 | −4.677 | −0.799 |
| UPI00022F8B4D | −3.661 | −2.642 | −0.956 | −4.680 | −0.675 |
| WP_055284109.1 | −1.184 | 2.314 | 1.852 | −4.682 | −0.968 |
| WP_128520904.1 | −1.789 | 1.105 | 2.418 | −4.682 | −0.680 |
| WP_106024518.1 | −4.014 | −3.280 | −0.553 | −4.748 | −0.956 |
| WP_004234437.1 | −2.575 | −0.330 | −0.310 | −4.819 | −0.774 |
| WP_073010654.1 | −3.807 | −2.773 | −0.529 | −4.841 | −1.414 |
| UPI0007B45EC7 | −3.538 | −2.135 | −1.289 | −4.940 | −0.604 |
| UPI0002314B74 | −2.227 | 0.503 | 0.506 | −4.957 | −0.886 |
| TCP18101.1 | −6.718 | −8.469 | | −4.966 | −0.819 |
| WP_107514794.1 | −3.187 | −1.374 | −0.848 | −5.000 | −0.780 |
| WP_072904346.1 | −3.012 | −0.983 | −1.345 | −5.041 | −0.760 |
| UPI000B5661AA | −2.702 | −0.312 | −0.125 | −5.091 | −1.071 |
| WP_047150996.1 | −3.889 | −2.674 | −0.477 | −5.104 | −1.491 |
| WP_092724975.1 | −2.237 | 0.735 | 1.158 | −5.210 | −0.836 |
| UPI00079B135B | −3.303 | −1.348 | −0.539 | −5.258 | −0.804 |
| WP_019108121.1 | −3.063 | −0.854 | −1.210 | −5.272 | −0.814 |
| WP_145458209.1 | −3.822 | −2.356 | −0.702 | −5.287 | −0.724 |
| WP_076065282.1 | −2.230 | 0.839 | 0.272 | −5.299 | −0.965 |
| UPI0001E35ACE | −4.480 | −3.642 | −0.681 | −5.319 | −0.711 |
| WP_080977968.1 | −4.570 | −3.807 | −0.873 | −5.333 | −0.891 |
| WP_093587584.1 | −1.297 | 2.771 | 4.679 | −5.365 | −0.904 |
| WP_098170605.1 | −2.272 | 0.844 | 2.230 | −5.389 | −0.877 |
| WP_148001988.1 | −5.237 | −5.016 | −0.970 | −5.457 | −1.152 |
| UPI000150D6AC | −3.004 | −0.463 | −0.403 | −5.545 | −0.758 |
| WP_073112630.1 | −4.727 | −3.891 | −0.617 | −5.563 | −1.144 |
| WP_067349107.1 | −3.758 | −1.938 | −1.023 | −5.577 | −0.839 |
| WP_127100780.1 | −3.046 | −0.440 | −2.025 | −5.652 | −0.952 |
| SUY49750.1 | −3.606 | −1.549 | −1.689 | −5.663 | −0.742 |
| UPI00015C01AE | −3.262 | −0.858 | −0.452 | −5.666 | −1.047 |
| UPI000009B019 | −6.879 | −8.056 | | −5.703 | −1.399 |
| WP_097006457.1 | −4.992 | −4.259 | −0.733 | −5.725 | −1.307 |
| WP_087290962.1 | −4.633 | −3.533 | −1.181 | −5.734 | −0.898 |
| UPI00000B3F97 | −3.138 | −0.503 | −1.380 | −5.773 | −1.126 |
| WP_112330076.1 | −2.625 | 0.565 | 0.375 | −5.815 | −0.919 |
| RPI78794.1 | −4.262 | −2.690 | −0.447 | −5.835 | −0.886 |
| WP_117768035.1 | −3.593 | −1.338 | −0.432 | −5.849 | −0.934 |
| WP_081735325.1 | −4.408 | −2.932 | −2.350 | −5.884 | −0.905 |
| RRG08833.1 | −5.191 | −4.466 | −0.701 | −5.916 | −1.175 |
| RDE19343.1 | −6.515 | −7.102 | −2.102 | −5.929 | −1.096 |
| WP_110990907.1 | −3.196 | −0.442 | −0.140 | −5.951 | −1.083 |
| SCQ72869.1 | −6.118 | −6.279 | −0.994 | −5.957 | −0.867 |
| WP_006845711.1 | −5.195 | −4.425 | −0.688 | −5.964 | −1.008 |
| WP_147265819.1 | −3.650 | −1.310 | −0.511 | −5.989 | −1.036 |
| WP_035430909.1 | −3.533 | −1.038 | −0.628 | −6.027 | −0.986 |
| WP_051624047.1 | −2.644 | 0.745 | 0.408 | −6.032 | −1.721 |
| AKT73182.1 | −2.584 | 0.868 | 0.712 | −6.036 | −0.929 |
| SCJ42694.1 | −3.703 | −1.337 | −0.698 | −6.070 | −0.834 |
| WP_025114396.1 | −4.043 | −1.981 | −0.329 | −6.106 | −1.313 |
| UPI00000105D3 | −6.946 | −7.779 | −1.250 | −6.114 | −1.204 |
| RZT66774.1 | −3.655 | −1.121 | −0.672 | −6.188 | −0.973 |
| WP_110092637.1 | −3.495 | −0.594 | −0.262 | −6.395 | −0.952 |
| WP_068202759.1 | −4.084 | −1.761 | −1.896 | −6.406 | −0.956 |
| WP_087225255.1 | −3.394 | −0.307 | −0.056 | −6.481 | |
| WP_051200279.1 | −6.481 | −6.481 | | −6.481 | |
| WP_061405262.1 | −4.491 | −2.468 | −0.973 | −6.514 | −1.107 |
| WP_099424140.1 | −3.048 | 0.427 | 0.447 | −6.524 | −1.210 |
| WP_030975214.1 | −3.064 | 0.448 | 0.210 | −6.577 | −1.182 |
| ERL63827.1 | −4.769 | −2.947 | −0.511 | −6.590 | −1.417 |
| PAV10712.1 | −4.703 | −2.785 | −1.134 | −6.622 | −1.370 |
| WP_108404827.1 | −5.193 | −3.731 | −0.574 | −6.656 | −1.610 |
| WP_079588582.1 | −5.563 | −4.285 | −0.748 | −6.840 | −1.230 |
| WP_051264703.1 | −4.070 | −1.229 | −0.520 | −6.910 | −1.386 |
| WP_032686941.1 | −4.539 | −2.144 | −1.524 | −6.933 | −1.193 |
| UPI0000030D3E | −4.940 | −2.882 | −0.474 | −6.997 | −1.447 |
| WP_021747387.1 | −4.416 | −1.833 | −2.092 | −6.999 | −1.192 |
| WP_006657622.1 | −3.670 | −0.340 | −0.087 | −7.000 | −1.112 |
| WP_015358111.1 | −4.137 | −1.273 | −0.894 | −7.001 | −0.946 |
| WP_026627303.1 | −6.274 | −5.364 | −1.014 | −7.184 | −1.057 |
| UPI000A08A794 | −5.479 | −3.757 | −0.629 | −7.200 | −1.353 |
| WP_094369469.1 | −4.771 | −2.249 | −0.663 | −7.293 | −1.765 |
| UPI0009F8F604 | −4.489 | −1.629 | −2.086 | −7.349 | −2.203 |
| WP_025706233.1 | −6.749 | −6.123 | −0.874 | −7.375 | −1.526 |
| UPI0007B642FE | −3.427 | 0.570 | 2.381 | −7.424 | −1.335 |
| UPI000009B628 | −7.537 | −7.628 | −1.701 | −7.447 | −1.443 |
| WP_118227047.1 | −3.873 | −0.273 | −0.044 | −7.473 | |
| UPI00005F0A78 | −7.690 | −7.814 | −1.324 | −7.567 | −1.196 |
| WP_111921306.1 | −5.739 | −3.745 | −0.581 | −7.734 | −1.301 |
| WP_019125538.1 | −4.137 | −0.535 | −0.174 | −7.740 | −1.199 |
| UPI0009F5E532 | −4.307 | −0.849 | −0.295 | −7.766 | −2.216 |
| WP_076170610.1 | −6.169 | −4.432 | −0.752 | −7.905 | −1.546 |
| WP_142511229.1 | −3.700 | 0.548 | 0.321 | −7.949 | −1.164 |
| WP_114014965.1 | −4.249 | −0.461 | −0.195 | −8.036 | −1.510 |
| WP_067024969.1 | −6.496 | −4.938 | −0.798 | −8.053 | −1.345 |
| WP_020135111.1 | −6.275 | −4.494 | −0.728 | −8.056 | |
| WP_116232802.1 | −4.873 | −1.673 | −1.388 | −8.073 | −1.172 |
| WP_146678271.1 | −5.013 | −1.825 | −0.708 | −8.201 | −2.012 |
| UPI0002B78B34 | −3.528 | 1.307 | 0.578 | −8.362 | −1.043 |
| WP_109196224.1 | −3.031 | 2.727 | 3.516 | −8.790 | |
| WP_118246619.1 | −7.244 | −5.698 | −1.064 | −8.790 | |
| UPI0002E4C0BF | −6.514 | −4.118 | −0.619 | −8.909 | −1.787 |
| WP_132305216.1 | −3.683 | 1.686 | 2.052 | −9.053 | |
| WP_073846185.1 | −6.122 | −2.969 | −0.509 | −9.275 | |
| WP_076079849.1 | −6.697 | −3.975 | −1.807 | −9.420 | −2.412 |
| WP_123609006.1 | −5.040 | −0.626 | −2.428 | −9.454 | −1.210 |
| UPI000B49B5D9 | −5.182 | −0.905 | −1.189 | −9.459 | −1.391 |
| WP_132412730.1 | −9.467 | −9.467 | | −9.467 | |
| WP_125777163.1 | −5.085 | −0.640 | −3.561 | −9.530 | −2.720 |
| RMD50745.1 | −8.768 | −7.746 | −2.190 | −9.789 | |
| UPI0009880690 | −10.167 | −10.167 | | −10.167 | |
| OLA20462.1 | −6.593 | −2.825 | −3.398 | −10.362 | −2.349 |
| WP_150051132.1 | −9.340 | −7.755 | −1.412 | −10.925 | |

UPI0000010203
(SEQ ID NO: 172)
ATNESLKNQLSTKKETGLGSAGNTIKGLMNSPAIKKRFEEVLKQR
APQYMSSIVNLVNSDINLKKCDQMSVVASCMVAATLDLPVDKNLG
YAWVVPYGNKAQFQLGYKGYVQLALRTGQYKSINVIEIHEGELID
WNPLTEELKIDFSKKESDAVIGYAGYFELLNGFKKSTYWTKEQIT
KHKNKFSKSDFGWKKDFDAMARKTVLRNMLSKWGILSIEMQNAYT
ADQGIIKNEIIETGEVKENIEYIEADFESYEDNSIEEGGANE
UPI00000105D3
(SEQ ID NO: 173)
ATNESLKNQLTTKKETGLGSAGNTIKGLMNSPAIKKRFEEVLKQR
APQYMSSIVNLVNSDINLKKCDQMSVVASCMVAATLDLPVDKNLG
YAWVVPYGNKAQFQLGYKGYVQLALRTGQYKSINVIEIHEGELID
WNPLTEELKIDFSKKESDAVIGYAGYFELLNGFKKSTYWTKEQIT
KHKNKFSKSDFGWKKDFDAMARKTVLRNMLSKWGILSIEMQNAYT
ADQGIIKNEIMETGEVKENIEYIEADFESYEDNSIEEGGANE
UPI0000030D3A/HAW2682705.1 RecT
[Escherichia coli]
(SEQ ID NO: 167)
TKQPPIAKADLQKTQGNRAPAAVKNSDVISFINQPSMKEQLAAAL
PRHMTAERMIRIATTEIRKVPALGNCDTMSFVSAIVQCSQLGLEP
GSALGHAYLLPFGNKNEKSGKKNVQLIIGYRGMIDLARRSGQIAS
LSARVVREGDEFSFEFGLDEKLIHRPGENEDAPVTHVYAVARLKD
GGTQFEVMTRKQIELVRSLSKAGNNGPWVTHWEEMAKKTAIRRLF
KYLPVSIEIQRAVSMDEKEPLTIDPADSSVLTGEYSVIDNSEE
UPI0000030D3E
(SEQ ID NO: 166)
STALATLAGKLAERVGMDSVDPQELITTLRQTAFKGDASDAQFIA
LLIVANQYGLNPWTKEIYAFPDKQNGIVPVVGVDGWSRIINENQQ
FDGMDFEQDNESCTCRIYRKDRNHPICVTEWMDECRREPFKTREG
REITGPWQSHPKRMLRHKAMIQCARLAFGFAGIYDKDEAERIVEN
TAYTAERQPERDITPVNDETMQEINTLLIALDKTWDDDLLPLCSQ
IFRRDIRASSELTQAEAVKALGFLKQKAAEQKVAA
UPI000009AF52
(SEQ ID NO: 174)
ANELGIFSVDNLNMTTIKQYLDGGGKASDAELVLLINLCKQNNMN
PFMKEVYFIKYGNQPAQIVVSRDFYRKRAFQNPNFVGIEVGVIVL
NKDGVLEHNEGTFKTHEQELVGAWARVHLKNTEIPVYVAVSYDEY
VQMKDGHPNKMWTNKPCTMLGKVAESQALRMAFPAEFSGTYGEEE
YPEPEKEPREVNGVKEPDRAQIESFDKEDYAAKKIEELKEKAQPQ
KEVVEETGEVIDEEPLEGF
UPI000009B019
(SEQ ID NO: 175)
STALATLAGKLAERVGMDSVDPQELITTLRQTAFKGDASDAQFIA
LLIVANQYGLNPWTKEIYAFPDKQNGIVPVVGVDGWSRIINENQQ
FDGMDFEQDNESCTCRIYRKDRNHPICVTEWMDECRREPFKTREG
REITGPWQSHPKRMLRHKAMIQCARLAFGFAGIYDKDEAERIVEN
TAYTAERQPERDITPVNDETMQEINTLLIALDKTWDDDLLPLCSQ
IFRRDIRASSELTQAEAVKALGFLKQKATEQKVAA
UPI000009B628
(SEQ ID NO: 176)
STNDELKNKLANKQNGGQVASAQSLGLKGLLEAPTMRKKFESVLD
KKAPQFLTSLLNLYNGDDYLQKTDPMTVVTSAMVAATLDLPIDKN
LGYAWIVPYKGRAQFQLGYKGYIQLALRTGQYKSINVIEVRDGEL
LKWNRLTEEIELDLDNNTSEKVIGYCGYFQLINGFEKTVYWTRKE
IEAHKKKFSKSDFGWKKDYDAMAKKTVLRNMLSKWGILSIDMQTA
VTEDEAEPRERKDVTEDESIPDIIDAPITPSDTLEAGSEVQGSMI
UPI000009BC15
(SEQ ID NO: 177)
STALATLAGKLAERVGMDSVDPQELITTLRQTAFKGDASDAQFIA
LLIVANQYGLNPWTKEIYAFPDKQNGIVPVVGVDGWSRIINENQQ
FDGMDFEQDNESCTCRIYRKDRNHPICVTEWMDECRREPFKTREG
REITGPWQSHPKRMLRHKAMIQCARLAFGFAGIYDKDEAERIVEN
TAYTAERQPERDITPVNDETMQEINTLLIALDKTWDDDLLPLCSQ
IFRRDIRASSELTQAEAVKVLGFLKQKASEQKVAA
UPI00000B3F97 Bet
[Gammaproteobacteria]
(SEQ ID NO: 178)
EKPKLIQRFAERFSVDPNKLFDTLKATAFKQRDGSAPTNEQMMAL
LVVADQYGLNPFTKEIFAFPDKQAGIIPVVGVDGWSRIINQHDQF
DGMEFKTSENKVSLDGAKECPEWMECIIYRRDRSHPVKITEYLDE
VYRPPFEGNGKNGPYRVDGPWQTHTKRMLRHKSMIQCSRIAFGFV
GIFDQDEAERIIEGQATHVVEPSVIPPEQVDDRTRGLVYKLIERA
EASNAWNSALEYANEHFQGVELTFAKQEIFNAQQQAAKALTQPLA
S
UPI000019AB49 Bet
[Escherichia coli]
(SEQ ID NO: 179)
STALATLAGKLAERVGMDSVDPQELITTLRQTAFKGDASDAQFIA
LLIVANQYGLNPWTKEIYAFPDKQNGIVPVVGVDGWSRIINENQQ
FDGMDFEQDNESCTCRIYRKDRNHPICVTEWMDECRREPFKTREG
REITGPWQSHPKRMLRHKAMIQCARLAFGFAGIYDKDEAERIVEN
TAYTAERQPERDITPVNNETMQEINTLLIALDKTWDDDLLPLCSQ
IFRRDIRASSELTQAEAVKALGFLKQKATEQKVAA
UPI000034E66D Bet
[Lactococcus phage phiLC3]
(SEQ ID NO: 180)
ANEIDIYDAKNLNTATVKKFLKGGGQASDEELAMLLAISRNQNMN
PFMKEVYFIKYGSAAAQIVVSRDFYRKRAFQNPNFAGIEVGVIVL
NKDGVLEHNEGTFKTKDQELVGAWARVHLKNTEIPVYVAVSYDEY
VQMKNGQPNSMWTNKPCTMLGKVAESQALRMAFPAEFSGTYGEEE
YPEPEKEPREVNGVKEPDRAQIESFDKENYAARKIEELKEKAQPQ
KEFVEEIGEAIDEITAEDF
UPI00005FOA78
(SEQ ID NO: 181)
STALATLAGKLAERVGMDSVDPQELITTLRQTAFKGDASDAQFIA
LLIVANQYGLNPWTKEIYAFPDKQNGIVPVVGVDGWSRIINENQQ
FDGMDFEQDNESCTCRIYRKDRNHPICVTEWMDECRREPFKTREG
REITGPWQSHPKRMLRHKAMIQCARLAFGFAGIYDKDEAERIVEN
TAYTAERQPERDITPVNDETMQEINTLLIALDKTWDDDLLPLCSQ
IFRRDIRASSELTQAEAVKALGFLKQKASEQKVAA
UPI000150D6AC
(SEQ ID NO: 182)
ANELGIFSVDNLNMTTIKQYLDGGGKASDAELVLLINLCKQNNMN
PFMKEVYFIKYGNQPAQIVVSRDFYRKRAFQNPNFVGIEVGVIVL
NKDGVLEHNEGTFKTHEQELVGAWARVHLKNTEIPVYVAVSYDEY
VQMKDGHPNKMWTNKPCTMLGKVAESQALRMAFPAEFSGTYGEEE
FPEPEKEPREVNGVKEPDRAQIESFDKEDYAAKKIEELKEKAQPQ
KEVVEETGEVIDEEPLEGF
UPI0001594E53
(SEQ ID NO: 183)
TTALQTLTNKLAERFEMGDGSGLVETLKSSAFAGATVSDAQMIAL
LVIANQYQLNPWTKEIYAFPGGNGGLTPIVGVDGWVRIINREPQY
DGMEFHFTDDYSACTCTIYRKDRSKPIVVTEFMGECKKSSPAWNS
HPKRMLRHKAMIQCARLAFGFTGIYDQDEAERIAENEKPPKNITP
QNNVVETTAVELISEEQLSQIRQLMQVTGTEEAKILAYIGVQALN
QIPKSQAEAVIKKLNLTLDKQNAEKADNGESVGEEIPL
UPI00015968D7
(SEQ ID NO: 184)
AKNELAKGSYLTDLQKLDGNTLRDFVDPKHQASPQELQALLAIVK
GRNLNPFTKEVYFIKYGSAPAQIVVSKEAIMKRAEENPDFDGFEA
GIVVETKDGAIERLTGTIVPKSATLRGGWCKVYRKDRSHAIEADA
DFAYYTTSKNLWQKMPALMIRKVAIVSAFREAFSESVGGLYTADE
MQRETQAEVRARKMKEAYEEKLYLLTQMEAKSYKKTKSKNENEAK
KTKEAEAIETVEEPTQDGNLEW
UPI00015C01AE
(SEQ ID NO: 185)
MAKENYSDPNGKLLNSITTFEVNGEEVKLSGNIIRDYLVSGNAEV
TDQEIIMFLQLCKYQKLNPFLNEAYLVKFKNTKGPDKPAQIIVSK
EAFMKRAETHEQYDGFEAGVIVERGGEIIELEGAVSLASDKLLGG
WAKVFRKDRNRPVSVRISEKEFNKRQSTWNTMPLTMMRKTAVVNA
MREAFPDNLGAMYTEEEQGSLQNTETSVQQEIKQNANAEVLDIPS
QQNEVPDFKEVREPEHVEMPPIYGEQQSTPPARPY
UPI00015C02E0
(SEQ ID NO: 186)
ATNDELKNQLANKQNGGQVASAQSLDLKGLLEAPTMRKKFEKVLD
KKAPQFLTSLLNLYNGDDYLQKTDPMTVVTSAMVAATLDLPIDKN
LGYAWIVPYKGRAQFQLGYKGYIQLALRTGQYKSINVIEVRDGEL
LKWNRLTEEIELDLDNNTSEKVIGYCGYFQLINGFEKTVYWTRKE
IEAHKKKFSKSDFGWKKDYDAMAKKTVLRNMLSKWGILSIDMQTA
VTEDEAEPRERKDVTEDESIPDIIDAPITPSDTLEAGSEVQGSMI
UPI00019E1F9A
(SEQ ID NO: 187)
PQEIAKVEYTAADGQEVRLTPGVIAKYIVSGNGLASEKDIYSFMA
RCQARGLNPLAGDAYMTVYQGKDGNTSSSVIVSKDYFVRTATAQD
SFDGMEAGVTVLNGQGQIQKREGCEFFPSLGEKLLGGWAKVHVKD
REHPSKAAVTMDEYDQHRSLWKSKPATMIRKVAIVQALREAYPGQ
FGGVYDRDEMPPSQEPQQVPVEVYEAPEAYETPDNQNRATEEF
UPI0001BEF484
(SEQ ID NO: 188)
NTPTMKRKFEEVLHENANAFMSNVMTLVSNDSYLAESEPMSILSG
ALTAATLNLGLDKNLGYAYLVPFNTKNKQTGKWERKAQFILGYKG
YIQLAQRSGKYKALNVIEVYEGELLSWNRLTEEFEFDPNGRQSDD
VIGYVGYFELLNGFKKTVYWTKQEIEAHRIANSKDKEKTKLSGVW
ATDYNAMARKTVLRNMLSKWGILSIEMQEATTSDEKVQQMQEDGN
IISETEVEENTTMKTAEVINEADSDSLNQTDLFDTKNPPLE
UPI0001CE597A CK3_26380
[butyrate-producing bacterium SS3/4]
(SEQ ID NO: 189)
ENATAVQQAESQGTQDFSAPVKHNTDFSLGIFGSSDNFLMATQMA
KAFASSTIVPKEYQGNFANGLVAMDIANRLKTSPFMVMQNLDVIQ
GRPAWRATFLIAMINRSKKYDIELQFEEKRDKNGKPYSCTCWTTK
DGRKVTGIEVTMDMAEAEGWTKKNGSKWITMPQVMLRYRAASFFS
RMNCPELSNGLYTTDEVYEMADSEYKVYNLEDEVKRDLAQNANKE
EFVAPPNETAPESESKGSEPLDPAVENQKSGDTPDWMKPETM
UPI0001D2DF22 RecT
[Cellulosilyticum lentocellum]
(SEQ ID NO: 190)
SDKKELVLKETHSRLNQLLATKMEAMPKDFNQTRFLQNCMTVLQD
TKGIENCHPVSIARTLLKGAFLGLDFFQRECYAIPYGGELQFQTD
YKGETKMAKKYSIRDIKDIYAKVVRKGDEFKEEIVAGQQVVDFKP
LPFNDAEIIGAFAVVLYQDGGMEYETMSTKQIEGIRDNFSKMKNG
LMWTKTPEEAYKKTVLRRLTKKIEKDFASIDQAKAYEESSDMQFK
QDEQKQDAKDPFADAVDVEFTEETEGQVRLDGEADGAK
UPI0001E0C499
(SEQ ID NO: 191)
SNELMTKAVTYEVNNEEVKLSGQIVKQYLTSGQAVTDQEVTMFIQ
LCRYQHLNPFLNEAYLVKFNGKPAQIITSKEAFMKRAESNPNYAG
LKAGCIVERNGELIYTEGAFTLKTDNILGAWADVIRKDRREPTHV
EISMDEFSKSQATWKSMPATMIRKTAIVNALREAFPQDLGALYTE
DDKNPNEATQTTYKQEPEVNTTKTADVLAKKFSGAPQIKSVENVQ
ESEEESNNASNHGEATEPVNNVEEPTATAEVEQGQLL
UPI0001E2AFC1
(SEQ ID NO: 192)
TNNQLATQIKRDITTDPSLLTGADIKKYFDPQNLLTEKQVGQALA
LCKGRNLNPFANEVYIVAYQNNSGTEFSLIVSKEAFMKRAERCEG
YDGFEAGITVMRNGEMIEIEGSLKLPEDILIGGWAVVYRKDRSHR
YKVTVDFNEYVKTDRNGNPRSTWKSMPATMIRKTALVQTLREAFP
DELGNMYTDIDGGDTFDAIKDVTPQESREDVVARKMAQIDQFNKE
QEANHADPEPTQNEDPIQGELLDGELEY
UPI0001E35ACE
(SEQ ID NO: 193)
TNNQIVEAKGDFLTNPQLLNSGIIRKYLDPQGKASDEELAYFIAQ
AKAQNLNPFTKEIYFIKYGTQPAQIVTAKSAFEKKADSHPQFDGK
EAGVIYLMDGEIKYSKGAFIPKGAEILGGWAKVYRKDRTYPTETE
VSFEEYDNSKIRARVKELTQQGKDVTYPVMNSYGKPIGENNWDTM
PCVMIRKVALVSAYREAFPAELGASYEADEIQLDNTPKDVTPQES
REDVVARKMAEIEQFNKEQEANHADPEPTQNEDPIQGELLDGELE
Y
UPI00020BA2E0
(SEQ ID NO: 194)
NNEVMEKSVEYEVNGNSVKLTPNMIKQFITKGNADVTDQEAIMFM
KLAEQQQLNPFLNEVYLIKFKGKPAQNIVAKEAFMKRAEKHSEYD
GLEAGIIVQRGEEIKELPGAVCLPTDNLLGGWARVYRKDRKNPFY
VQLDFKEFSKGQATWNQMPKNMIRKTAIVNALREAFPEALGAMYT
EDDARLEEVKTAEPIKEKAETTQILENKFKELSENGQTEVGDEQT
NESTEPEPTAKQEQLL
UPI000212F382
(SEQ ID NO: 195)
TVQLVQPRNSDEYDFDQTKLDLIKRTICKGATNDELQLFIHACKR
TGLDPFMRQIFAVKRWDSSTKKEIMTIQTGIDGYRLIADRTGKYA
PGKDTEFGYDNKGNIRWAKAYIKKMTPDGQWHEISAIAFWEEYVQ
TTREGKSTLFWLKKSHIMLSKCTEALALRKTFPAERSGIYTKEEM
AQEFSPLEEHLVERIAASRNDQGRS
UPI00022F8B4D
(SEQ ID NO: 196)
SNNQLSTQQAKRDIAIDTSVWTFQDVKRYFDPQNLLTEKQVGQAL
SLIKGRNLNPLANEVYIVAYKKKTGGTEFSLIVSKEAFLKRAAQN
PNYEGFEAGVVTVDTDGVMHERKGALMLPGDTLVGGWARVYRKNF
KVPVEIFVSREEYDKKQSTWNAMPATMIRKTALVNALREAFPEDL
GNMYTEDDGGETFDRIKQAEPVESREDVMARKMAQIEQMKQEQAQ
RQIDTSYPTDDVIDPDDEPAQGELLEDLEY
UPI0002314B74
(SEQ ID NO: 197)
AITPNPIPAQDGSPIPSPDDIVGELARRKIYAGIPDDDVALALAL
CQKYGFDPLLKHLVLLATKDRDETTGQGQKHYNAYVTRDGLLHVA
HTSGMLDGLETIQGKDDLGEWAEAVVYRKDMSRPFRYRVYLSEYV
REAKGVWKTHPQAMLTKTAEVFALRRAFDVALTPFEEMGFDNQNI
AGDTGPSPKTGFTEKAGFTGNTDFSAEASLPGKARFSTEAGLTDM
TVIPPNRVTGSIPETSRLNTSAGSTGRQRRQLF
UPI00025CAD2E
(SEQ ID NO: 198)
STALATLAGKLAERVGMDSVDPQELITTLRQTAFKGDASDAQFIA
LLIVANQYGLNPWTKEIYAFPDKQNGIVPVVGVDGWSRIINENQQ
FDGMDFEQDDESCTCRIYRKDRNHPICVTEWMDECRRAPFKTREG
REITGPWQSHPKRMLRHKAMIQCARLAFGFAGIYDKDEAERIVEN
TAYTTERQPERDITPVNEETMSEINALLTSMEKTWDDDLLPLCSQ
IFRRYIRASSELSQAEAEKVLGFLKQKATEQKVAA
UPI00025CF49A
(SEQ ID NO: 199)
EKPKLIQRFAERFSVDPNKLFDTLKATAFKQRDGSAPTNEQMMAL
LVVADQYGLNPFTKEIFAFPDKQAGIIPVVGVDGWSRIINQHDQF
DGMEFKTSETKVSLDGAKECPEWMECIIYRRDRSHPVKITEYLDE
VYRPPFEGNGKNGPYRVDGPWQTHTKRMLRHKSMIQCSRIAFGFV
GIFDQDEAERIIEGQATHVVEPSVIPPEQVDDRTRGLVYKLIERA
EASNAWNSALEYANEHFQGVELTFAKQEIFNAQQQAAKALTQPLA
S
UPI0002AD92E7
(SEQ ID NO: 200)
TTVNQTELKNKLAEKAKTPAKTGNTVFDLIRKMEPEIKRALPKQI
SPERFARIAMTAVRNTPKLQACEPISFIAALMQSAQLGLEPNTPL
GQAYLIPYGKEVQFQLGYQGMLTLAYRTGEYQSIYAMPVYANDEF
EYEYGLNEKLVHKPAPDPEGEPIYYYAVYKLKNGGHGFVVMSRQQ
IERHRDKYSPSAKQGKFSPWNTDFDSMAKKTVLKQLLKYAPKSVE
FATQIAQDETIKTEIAEDMTEVQGIEVEYEATDDQENQENQEQED
UPI0002B78771
(SEQ ID NO: 201)
EFETDEEEKEMSNNQLSTQQAKRDIAIDTSVWTFQDVKRYFDPQN
LLTEKQVGQALSLIKGRNLNPLANEVYIVAYKKKTGGTEFSLIVS
KEAFLKRAAQNPNYEGFEAGVVTVDTDGVMHERKGALMLPGDTLV
GGWARVYRKNFKVPVEIFVSREEYDKKQSTWNAMPATMIRKTALV
NALREAFPEDLGNMYTEDDGGETFDRIKQAEPVESREDVMARKMA
QIEQMKQEQAQRQIDTSYPTDDVIDPDDEPAQGELLEDLEY
UPI0002B78B34
(SEQ ID NO: 202)
TTNQVVTHKNFFNAPNVQKSFDDVWKGAGVQFATSILSVIQGNAS
LKSASNESIMTSAMKAAVLNLPIEPSLGRAYLVPYKGQVQFQLGY
KGLIELAQRSGKYKSINAGPVYKSQFVSYDPLFEELTLDFTQPQD
EVIGYFASFSLLNGFRKLTYWTKAEVEAHGKKFSKTFGNGPWKTD
FDAMARKTVLKHILSIYGPLSVEMQTGMQNDESENDNATRDIKTA
EPVNADQQLLEDLMNVDTETGEILEEVSELKDNGELDLKYEDPNA
R
UPI0002B884F0/WP_003158887.1 Bet
[Pseudomonas aeruginosa]
(SEQ ID NO: 203)
GTALTPLLTKFATRYEMGTTPEEVANTLKQTCFKGQVNDSQMVAL
LIVADQYKLNPFTKELYAFPDKNNGIVPVVGVDGWARIINENPQF
DGMEFSMDQQGTECTCKIYRKDRSHAISATEYMAECKRNTQPWQS
HPRRMLRHKAMIQCARLAFGFAGIYDQDEAERIVERDVTPAEQYE
DVSEAICLIKDSPTMEDLQSAFSNAWKAYKTKGARDQLTAAKDQR
KKELLDAPIDVEFEETGDDRAA
UPI0002CB4A67/WP_010792303.1 Bet
[Pseudomonas aeruginosa]
(SEQ ID NO: 204)
GTALTPLLTKFATRYEMGTTPEEVANTLKQTCFKGQVNDSQMVAL
LIVADQYKLNPFTKELYAFPDKNNGIVPVVGVDGWARIINENPQF
DGMEFSMDQQGTECTCKIYRKDRSHAISATEYMAECKRNTQPWQS
HPRRMLRHKAMIQCARLAFGFAGIYDQDEAERIVERDVTPAEQYE
DVSEAVCLIKDSPTMEDLQSAFSNAWKAYKTKGARDQLTAAKDQR
KKELLDAPIDVEFEETGDDRAA
UPI0002E4COBF
(SEQ ID NO: 205)
SSIAAAAESAEVTPASIINKYRDDIATVLPPKLRERIDRWIRLAI
GAVNSNPELISRVRADQGASMMQALMKCAALGHEPGSGLFHLVPK
GSRIEGWEDYKGILQRIDRSGVYARTVIGVVYANDEYSYDQNVDE
RPRHVRATGDRGEPISSYAYAVYPSGAITTVAEATPEQIASSKSK
ARGADNAASPWRAPGAPMHRKVAVRLLEKHVATSAEDRREPISRS
AANDVVIDATADYYQEP
UPI0003282677
(SEQ ID NO: 206)
TNELTQTKGAYLTDLQKLDGATLRNFVDPKHQASPQELQTLLAIV
KNRNLNPFTKEVYFIKYGNNPAQIVVSKDAFMKRAEQNQNYDGFE
SGIIYEDASGELKNKKGVILPKNCTLIGGWCEVYRKDRTRPVYRE
VELSAYNTGKNWWQKAPGQMIEKVAIVAAVRDSFSEDVGGLYTSE
EMEQAAPIDVTPQESQEEVRTRKMAQIEEMKREQEKHQSSAYPED
EIPNFEDEPLQGELLEEMEY
UPI00033853AF
(SEQ ID NO: 207)
NERTNLQYAPAPVERFKECLNSHEIKARLKNSLKNNWTQFQTSML
DLYSGDAYLQKCDPMAVALECVKAATLDLPISKSLGFAYVVPYNN
VPTFTLGYKGLIQLAQRTGQYRTINADVVYEGEIRGADKLSGMVD
LSGERTGDEVVGYFAYFKLINGFEKMIYMTRAEAEKWRDDYSPSA
KSKYSPWRTDFDKMALKTCIRRLISKYGIMSVEMQGVMTEEAEPR
AAAAAKRAEETVQANANSKVIDIDAAPPAANESPAEAAPQPDF
UPI0003427695
(SEQ ID NO: 208)
DYVTKIQEVLNRLLDAKHDALPSGFKKTRFSENCRAYVKEYTDLQ
KYDEEEVALVLFKGAVLGLDFLAKECHVITEGSALRFQTDYKGEM
ALVKKYSVRPILDIYAKNVREGDVFREEISEGKPLIHFNPLAFNN
SQIIGSFAVALFSDGGMVYETMPAEEIESIRRNYGKNPGSDTWEK
SQGEMYKRTVLRRLCKTIEIDFDAEQSLAYEAGSSFEFNREPQPK
KRSPFNPPEVEESEVLSDDGTSEAE
UPI000353091F
(SEQ ID NO: 209)
SNALTITQDQTEFTPKQLSVLENLGVQGAAPQEVAMFFDYCQRTG
LSPWARQIYMIGRWDRNLGRKKYAVQVSIDGQRLVAERSGVYEGQ
TAPQWCGPDGQWVDVWLANEPPQAARVGVWRKSFREPAYGVARLS
SYMPVTRDGKPQGLWGTMPDVMLAKCAESLALRKAFPLELSGLYT
SEEMQQADAPRTEPAPVDEDVVDAEIVDDEERMQWVEAIQAAETT
DVLRKMWADIKTCPDALQAELRELIPARAKELAA
UPI000386D631
(SEQ ID NO: 210)
IECAKLGLEPNNILGQAYLVPVCVDGVNKVEFQLGYKGLIELAYR
SGKIKSLYANEVFEKDEFHIDYGLDQKLIHKPFLGGDRGEVIGYY
AVYQMDNRGASFVFMTRDEILGHSRKYSRSFGCDLWESEFDAMAK
KTVIKKLLKYAPLSIELQKSVSVDESVKGIGCIGVI
UPI0003E3D237
(SEQ ID NO: 211)
GTALTPLLTKFATRYEMGTTPEEVANTLKQTCFKGQVNDSQMVAL
LIVADQYKLNPFTKELYAFPDKNNGIVPVVGVDGWARIINENPQF
DGMEFSMDQQGTECTCKIYRKDRSHAISATEYMAECKRNTQPWQS
HPRRMLRHKAMIQCARLAFGFAGIYDQDEAERIVERDVTPGEPVE
DVTEALSLINSAPTMDDLQAAFSDAWKAYKSKGARDQLTVAKDQR
KKELLEAPIDVEFEETGDDRAA
UPI00044F7143
(SEQ ID NO: 212)
STALATLAGKLAERVGMDSVDPQELITTLRQTAFKGDASDAQFIA
LLIVANQYGLNPWTKEIYAFPDKQNGIVPVVGVDGWSRIINENQQ
FDGMDFEQDNESCTCRIYRKDRNHPICVTEWMDECRREPFKTREG
KEIIGPWQSHPKRMLRHKAMIQCARLAFGFAGIYDKDEAERIVEN
TACTAEHQPERDITPVNDETMQEINTLLIALDKTWDDDLLPLCSQ
IFRRDIRASSELTQAEAVKALGFLKQKATEQKVAA
UPI0004995B90
(SEQ ID NO: 213)
TNNQLATQTKRNITTDPSLLTGADIKKYFDPQNLLTEKQVGQALA
LCKGRNLNPFANEVYIVAYTNRNGGKEFSLIVSKEAFLKRAAQCK
DYEGFEAGVVVVDSEGVMHERKGAIMLPEDTLIGGWARVHRKNFK
VPVEIFVSREEYDKKQSTWNTMPATMIRKVALVNALREAFPEDLG
NMYTEDDGGETFDRIKDVTPQESREDVVARKMAQIEQFNKEQEAN
HADPEPAQTEETIQGELLDGELEY
UPI00051F5876
(SEQ ID NO: 214)
SNREIEVIRACSKAGNNGGSSPWDSFPDEMARKAIVKRASKYWPR
RDRLDTAIDYLNTQGGEGIILNADHIPERDVTPASDEIINEITQA
ITEINKTWDDLLPLCSKTFRRTIASHEYLSQEEAVKTLDFVKKKA
ARNKATAEAKIHATTENNSEAVS
UPI000588C848
(SEQ ID NO: 215)
ATNDELKNKLANKQNGGQVASAQSLDLKGLLEAPTMRKKFEKVLD
KKAPQFLTSLLNLYNGDDYLQKTDPMTVVTSAMVAATLDLPIDKN
LGYAWIVPYKGRAQFQLGYKGYIQLALRTGQYKSINVIEVREGEL
LKWNRLTEEIELDLDNNTSEKVVGYCGYFQLINGFEKTVYWTRKE
IEAHKKKFSKSDFGWKKDYDAMAKKTVLRNMLSKWGILSIDMQTA
VTEDEAEPRERKDVTDDESIPDIIEAPITPSDTLEAGSVVQGSMI
UPI000598CD40
(SEQ ID NO: 216)
TNNQLATQTKRNITTDPSLLTGADIKKYFDPQNLLTEKQVGQALA
LCKGRNLNPFANEVYIVAYTNRNGGKEFSLIVSKEAFLKRAAQCK
DYEGFEAGVVVVDSEGVMHERKGAIMLPEDTLIGGWARVHRKNFK
VPVEIFVSREEYDKKQSTWNTMPATMIRKVALVNALREAFPEDLG
NMYTEDDGGETFDRIKDVTPQESREDVVARKMAQIEQFNKEQEAN
HADPEPAQTEEPIQGELLDGELEY
UPI0005DCEBAD
(SEQ ID NO: 217)
TNNQLATQTKRNITTDPSLLTGADIKKYFDPQNLLTEKQVGQALA
LCKGRNLNPFANEVYIVAYTNRNGGKEFSLIVSKEAFLKRAAQCK
DYEGFEAGVVVVDSEGVMHERKGAIMLPEDTLIGGWARVHRKNFK
VPVEIFVSREEYDKKQSTWNTMPATMIRKVALVNALREAFPEDLG
NMYTEDDGGETFDRIKDVTPQESREDVVARKMAEIEQFNKEQEAN
HADPEPAQTEESIQGELLDGELEY
UPI0005E4CB74
(SEQ ID NO: 218)
TNNQLATQTKRNITTDPSLLTGADIKKYFDPQNLLTEKQVGQALA
LCKGRNLNPFANEVYIVAYTNRNGGKEFSLIVSKEAFLKRAAQCK
DYEGFEAGVVVVDSEGVMHERKGAIMLPEDTLIGGWARVHRKNFK
VPVEIVVSREEYDKKQSTWNTMPATMIRKVALVNALREAFPEDLG
NMYTEDDGGETFDRIKDVTPQESREDVVARKMAEIEQFNKEQETN
HADPEPAQTEETIQGELLDGELEY
UPI0005FEB4BO
(SEQ ID NO: 219)
NEIQAYDKINDRDGMEMLGAAIQRSGMFGAETKEQGIILALQCMV
EKKPPLEMAKNYHIIQGKLSKRADAMLADFRKAGGKFIFADLKNP
TVQKAKVTFEDYKDFDVEYSIDDAKTAGVYNAKGAWVKYPGAMLR
ARLVSETLRAIAPEIVTGVYTPEELETPINAKPELKCAQPVKAKP
EPKKAQPDVIEATVCESELDAKLVELIGDREQIVNLYWEKKGLID
GLDTTWRDLNDDTKRKMIDQFDQFMDAAQRKAAQ
UPI00062002D2
(SEQ ID NO: 220)
AENEKQALLQEENKSENVVSTVKRTALATNPFSDTDQFNNIFKMA
QLISQSDMIPATYKGKPMNCVIALEQANRMGVSPLMVMQNLYVVK
GVPSWSGQGCMMIIQGCGKFRDVDYVYSGEKGTDSRSCKVVATRI
SDGKRIEGTEITMQMVKSEGWISNTKWKNMPEQMLGYRAATFFAR
MYCPNELNGFATEGEAEDMNHKPQRIEAINVLGDTAHE
UPI00064B44C1
(SEQ ID NO: 221)
TIMDLLNDPKMKSQIQRALPNGMSAERIARIALTALRMNPQLQEC
SPQSFAAALMTSAQLGLEPNTPLGHAWLIPRKNHGKMEVQFELGY
KGMLDLVRRSGMITAIFAEEVREKDEFEFEYGTNPYLKHKPYLGG
DRGKVLFYYAVATFKDGGYAFKVMSIPEIEEARKLSQSANSPYSP
WNRFYDEMAKKTVLKRLCKYLPLSIEVQRNLAQDETIRTQIEADD
ILDLPNENEFEVVEVEEIPGEEEKEEAKEGPFPNKALRESPTPLT
UPI00064D5E13
(SEQ ID NO: 222)
STALTTLTSQLSQRFKLDGGEELLTTLKQTAFKGQVTDAQMTALL
IVANQFGLNPWTKEIYAFPDKNGGIVPVVGVDGWARIINEHPQFD
GMDFEMDGEQSCTCVIYRKDRTRPIRITEYMAECKKTGGGPWQSH
PRRMLRHKAMIQCARMAFGFGGISDEDDAERIREKDITPQAEVVP
KALEPYPADKFEENFEQWKSLIESGRRSADDVIAKIKSRNTMTDE
QETRLRACGGEEGKTYENA
UPI00065C2D47 Bet
[Pseudomonas phage] PS-1
(SEQ ID NO: 223)
SNVATIKPSSLSARMAERFGVDPNEMMATLKATAFKGQVSDAQMQ
ALLIVADQYGLNPWTKEIYAFPDKGGIVPVVGVDGWSRIINENGA
FDGMDFQQDDESCTCIIYRKDRNHPIKVTEWMAECKRNTQPWQSH
PKRMLRHKAMIQCARLAFGYTGIFDEDEAQRIVEKDVTPAVNEPD
ITPALEAIKNASSMEELHAAFKAAWNQHPSARARLTAVKDERKKA
LSEPIEGELVENEDGPAQQ
UPI00067A7349 RecT
[Streptococcus phage] APCM01
(SEQ ID NO: 224)
AKNELVKGEYLTDLQKLDGNTLRNFVDPKHRASPQELQALLAIVK
NRNLNPFTKEVYFIKYGSAPAQIVVSKEAIMKRAEENPNFDGFEA
GIVIETKSGSIERLTGTIAPKRAELRGGWCKVYRKDRSHAIEADA
DFAYYTTGKNLWQKMPALMIRKVAIVSAFREAFSESVGGLYTADE
MEQNNTQETQEEVRARRMKQAYEEKLRLLTEMEAKSYKKVEDESA
SKEIEAAKTTKNTKEVEVIEETEVTEEPTQEDSLEW
UPI0006CE3F5D
(SEQ ID NO: 225)
STALATLAGKLAERVGMDSVDPQELITTLRQTAFKGDASDAQFIA
LLIVANQYGLNPWTKEIYAFPDKQNGIVPVVGVDGWSRIINENQQ
FDGMDFEQDNESCTCRIYRKDRNHPICVTEWMDECRREPFKTRDG
REITGPWQSHPKRMLRHKAMIQCARLAFGFAGIYDKDEAERIVEN
TTYTTDRQPERDITPVSDETMREINDLLITMNKTWDDDLLPLCSQ
IFRRDIGASSDLTQIEAVKALGFLKQKAAEQKVEA
UPI00078E90BE RecT
[Pirellula] sp. SH-Sr6A
(SEQ ID NO: 226)
SEATKEAKPETAIAKKPNGIKDWLKSDALKTQIASVAPKHMAPER
VMRIALNAVSRTPKLADCTPESFMRCLLDLSSWGLVPDGRHAHLI
PYGTECTLVLDYKGLVTLAYRSGWVKKIHADVVFEGDIFVYSLGT
VCQHIPWEFRDDANKPEHKGHFRAAYCVVTMADGIEKHEVMTASE
IDAIKAKSRSGNSGPWKDHYTEMAKKTVYRRASKWLPLSPEQADA
MERDDDRIIDAVSVAVTQRLSKAAMPLIGANETGDTE
UPI00078EBE91 RecT
[Pirellula +9sp. SH-Sr6A
(SEQ ID NO: 227)
SEATKEAKPETAIAKKPNGIKDWLKSDALKTQIASVAPKHMAPER
VMRIALNAVSRTPKLADCTPESFMRCLLDLSSWGLEPDGRHAHLI
PYGTECTLVLDYKGLVTLAYRSGWVKKIHADVVFEGDIFVYSLGT
VCQHIPWEFRDDANKPEHKGHFRAAYCVVTMADGIEKHEVMTASE
IDAIKAKSRSGNSGPWKDHYTEMAKKTVYRRASKWLPLSPEQADA
MERDDDRIIDAVSVAVTQRLSKAAMPLIGANETGDTE
UPI00078ED021
(SEQ ID NO: 228)
SEIQQQAEAQTQAHPTAVLDDYRGAIASVAPPGTNIDLFIRMTKS
NVNRSDEIVAAVKRNPGLFMQAVMDSAALGHIPGSEYYYLTPRRD
GISGIESWKGVAKRIFNTGRYQRIVCEVVYEGEQWEFQPGEDLKP
KHVIDWDARQVGSKVRFTYAYAVDFEGNPSTVAVCTKLDLDKAQK
QSRGKVWDQWYEQMAKKTAIKRLEDFVDTSAVDLRADGSSRRHSA
EVAE
UPI000795D815
(SEQ ID NO: 229)
ASKNEAIEVSPAEIASVKEKPASIVKAEKAKKEPCALVKYEDAEG
REVVLTREDIINTISSNPRITDKEIKLFIELARAQKLNPFTREIF
ITKYGDYPATFIVGKDVFTKRAQSNPLFKGMQAGIIVQRGNAVDQ
REGSATFGDEMLIGGWCKVYVQGYDVPIYDSVSFNEYAARKTDGT
LNAMWASKPATMIRKVAIVHALREAFPSDFQGLYDQSEMGLSGQG
GE
UPI00079B135B
(SEQ ID NO: 230)
ATSLKRAVTGDGKPATVQQLLTNPKIKSQIALALPQHLTPERLTR
IVLTEIRRTPALAKCKPESLLAAVMQCAQLGLEPGGSLGHAWLMP
FKNEVQFIIGYRGMIDLARRSGQVLSIEARGVYESDTFHVSFGLE
PDLTHQPDWDPADRGKLAFVYAVARLKDGGFQFDVMSRAEVEKIR
AQSPAGKSGPWVTHFEEMAKKTVIRRLFKYLPVSVEMARAVGLDE
AAERGEQSDAIDADCVIESEEEATPEEKGDSAA
UPI0007B45EC7
(SEQ ID NO: 231)
SEISKAVATQQNPLAVVARYKRELGTVLPTVLRQDPDRWLMAAEN
AARKNPDIMAVTKADQGASYMRALVECARLGHEPGSKDFHFIKRG
NAISGEESYRGIIKRVLNSGFYRSVVARTVFSNDTYSFDPLTDIV
PNHVPAQGDRGKPLSAYAFAVHWDGTPSTVAEATPERIATAKAKS
FASDKPTSPWQLPTGVMYRKTAIRELEPYVHVAPEPQPRRHLDGT
VGGIPATDFDVDDGDVLDITADQLAEAGEIV
UPI0007B642FE
(SEQ ID NO: 232)
SELQQAAQGQADAGPVQVIYSHAKEIQNVLAKGTDMDRWLQMARL
AVMRDPNLVNAAKRDPGSLMQAMLDCAEKGHIPGTEDYYLVPRKG
GIQGMESWKGIAKRIMRSGRYQSIVAEVVYEGEDFDFNPNTMDRP
VHQIKYMARTSGQPVLSYAYAVDHEGKPSTIAVADPRYIAKVKAN
SKGTVWADWDEAMYKKTAVKMLVDYVDTSSTDRRGVSTVQVDGPV
GTFIDGVLEIEGGDQ
UPI0007B64693
(SEQ ID NO: 233)
SELQQAAQGQQSNNPVSFIYSHAKDIQNVLTKGTDMDRWLQMARL
AVMRDQNLVASAKRDPGSLMQALLDCAEKGHVPGTEDYYLVPRKG
GIQGMESWKGIAKRIMRSGRYQSIVNEVVYEGETFEFNPNTMDRP
VHNINYMTRTSGKPVMSYAYALDHDGKPSSVAIADPRYIAKVKKN
SRGSVWEDWDEQMYRKTAVKMLQDYVDTSSVDRRDVSTVQVDDAN
VIDVDDTFTAREAGE
UPI0007BCAEAB
(SEQ ID NO: 234)
TQDLATAIADQQPAQRRTAFDLVESMRGELHKALPEHASIDNFLR
LALTELKMNPQLGNCSGESLLGALMTAARVGLEVGGPLGQFYLTP
RRLKRDGWAVVPIVGYRGLITLARRAGVGQVNAVVVHEGDTFREG
ASSERGFFFDWEPAVERGKPVGALAAARLAGGDVQHRYLSLAEVH
ERRDRGGFKDGSNSPWATDYDAMVRKTALRALVPLLPQSTALSFA
VQADEQVQRYDAGDIDIPALDETDTEDTK
UPI0007F13B78
(SEQ ID NO: 235)
TNQLAHKDFFNTPAVKQKFQEVLNGNERQFTASLLSIVNNNKLLA
RASNTSIMTAAMKAAVLNLPIEPSLGFAYIVPYGQDAQFQLGYKG
LIQLAIRSGQFKAINSGKVYKAQFKSYDPLFETLDIDFTQPEDEV
YGYFATFELVNGFKKLTFWTKEQAESHGKRFSKTYAKGPWSTDFD
AMAQKTVLKSILSKYAPLSTEMQEGLISDNQTEEVETDPIDVTPK
NEDTQTLLSDLMSDEAESETEKV
UPI000865F43D
(SEQ ID NO: 236)
TSQQLDTTHTINQQVTTFRHTLVQMKNEIAAALPAHMTGDRFLRL
ILTEVRKNPELAECSTESIFGGILTAAALGLEPGLNGECWLIPRK
VGKGPGSRKEATFQVGYKGIIKLFWQNPLASYLDTGVVYANDAWK
FRKGLDPILEHTPATGDRGAVRGYYAVVGLTTGARIFDFFTPKQI
SALRGTAGPNGGISDPEHWMERKTALLQVMKMAPKSTDLASAASV
DGTVQTVEAAAQVAAASTGPVNPTTGEVLEAEPVEGGAA
UPI000865FB15
(SEQ ID NO: 237)
TQQMPIEAQGEPTKELQQKAAVDRFNATLHQMQNEIARALPKHMT
GDRFVRIVLTEVRKDPTLALCDPLTMFGSLLTAAALGLEPGLNGE
CWLVPRKNHGTLEAQLQVGYRGVVKLFWQNPAAAYLDTGYVCERD
HFRFAKGLNPILEHTPAEGDRGKVVRYYAVAGLNTGARVFDVFTP
AQIKTLRGGKVGSNGDIPDPEHWMERKTALLQVLKLMPKSTQLAA
VPAADGRAHTISDAQQIFGGVDTSTGEVLEAEPVEGDAA
UPI0008D18539
(SEQ ID NO: 238)
ETIDIKQELASQAQTDSKKEVKLTKAMSIAEMIKAMMPEIKRALP
SMITPERFTRIALSALNNTPELQACTPMSFISALLNAAQLGLEIN
SPLGHAYLIPYKNKGVLECQFQIGYLGLIALAYRNELMQTIQAQC
VYENDEFLYEYGLNPKLVHRPATSDRGEPVFFYGLFKMINSGFGF
CVMSKQEMDEFARTYSKGLASSFSPWKTSYNEMAKKTVIKQALKY
APIKTDFQKALSTDESIKYAISEDMTEAVNEIVSQNTEVA
UPI0008D990CB
(SEQ ID NO: 239)
SNLKNQLANKAGGTATKKQPQTMQDWIKVMEPQIKKALPSVITAE
RFTRMALTAISTNPKLAECTPESFMGALMNAAQLGLEPNTPLGQA
YLIPYGKSVQFQVGYKGLMELAQRSGQFKSIYAHTVYENDEFEVE
YGLTQNIVHKPNFDDRGKPIGFYAVYKLTNGGENFVFMTQREVEE
FGKAKSKTFNNGPWKTDFEAMAKKTVLKQLLKYAPIKVEFQREIA
QDATIKTEIAEDMTEVPEEMVEAEYEVVEQNTMAEDADLKGTPFE
TK
UPI0008E12231
(SEQ ID NO: 240)
SNNELLAKPVEFEVNGEAVKLTGKTVKNFLVSGNGEVSDQEVVMF
INLCKYQKLNPFLNEAYLVKFKSKSGPDKPAQVIVSKEAFMKRAE
KHPNYEGFEAGIIVERDGQLVDIEGAIKLTNDKLVGGWARVYRSD
RQKPITTRISLSEFSKGQSTWNSMPLTMIRKSAIVNAQREAFPET
LGALYTEDDAKLDTTSSHDQEQVIEQEIKTKANQEVIDVEYTEES
EQKSPQQEQTETTQAGPGF
UPI0008EA8633
(SEQ ID NO: 241)
ATNSSLKNQLSKKENVTIGNTMQGLLNNPKMKKRFEEILDKKAPQ
YMSSILNLYNGDTSLQKCEPMSVLSSSMIAATMDLPVDKNLGYAW
IVPYKNKAQFQMGYKGYIQLALRTGQYKHINAIEIHEGELVNWNP
LTEELEIDFTKKESDKIIGYAGYFELLNGFKKSTYWTKTQIENHR
KKFSKSDYGWNKDFDAMAIKTVIRNMLSKWGILSIEMQNAYTADE
NIIKDSFIDDSENVSANIEDLVEADYTVNQDSLESKEEFEGTPLE
UPI00091F1EBO
(SEQ ID NO: 242)
KKMTVMKTSAPLCYADVAEVKCEEFYEDQYKAGAEELFDNTSYDR
LKVYLEKHGGLEGVHADVVRAGDTFVYRPGVIRRHGYVPGEQRGQ
VYAVYAKAHIKGGATRCVILARHEVEIDMDAKHGGNPDGDWENLA
KVVALRSLAEALPLPSAVLQSCRTWSAK
UPI000958E115
(SEQ ID NO: 243)
SNPPLAQADLQKTQGTEVKTKTKDQQLIHFINQPSMKAQLAAALP
RHMTPDRMIRIVTTEIRKTPALANCDMQSFIGAVVQCSQLGLEPG
NALGHAYLLPFGNGKAKSGQSNVQLIIGYRGMIDLARRSNQIISI
SARTVRQGDSFHFEYGLNENLTHVPGENEDSPITHVYAVARLKDG
GVQFEVMTHNQIEKVRASSKAGQNGPWVSHWEEMAKKTVIRRLFK
YLPVSIEMQKAVILDEKAEANVDQENASVFEGEFEEVSQSA
UPI0009805C1D
(SEQ ID NO: 244)
ANELGIFSVDNLNMTTIKQYLDGGGKASDAELVLLINLCKQNNMN
PFMKEVYFIKYGNQPAQIVVSRDFYRKRAFQNPNFAGIEVGVIVL
NKDGVLEHNEGTFKTHEQELVGAWARVHLKNTEIPVYVAVSYDEY
VQMKDGHPNKMWTNKPCTMLGKVAESQALRMAFPAEFSGTYGEEE
LPEPEKEPREVNGVKEPDRAQIESFDKEDYAAKKIEELKEKAQPQ
KEVVKETGEVIDKITAEDF
UPI0009805F63
(SEQ ID NO: 245)
ANELGIFSVDNLNMTTIKQYLDGGGKASDAELVLLINLCKQNNMN
PFMKEVYFIKYGNQPAQIVVSRDFYRKRAFQNPNFAGIEVGVIVL
NKDGVLEHNEGTFKTHEQELVGAWARVHLKNTEIPVYVAVSYDEY
VQMKDGHPNKMWTNKPCTMLGKVAESQALRMAFPAEFSGTYGEEE
LPEPEKEPREVNGVKEPDRAQIESFDKEDYAARKIEELKEKAQPQ
KEVVKETGEVIDEITAEDF
UPI0009880690
(SEQ ID NO: 246)
TNNQLVEAKGDFLTNPQLLNSGIIRKYLDPQGKASDEELAYFIAQ
AKAQNLNPFTKEIYFIKYGTQPAQIVTAKSAFEKKADSHPQFDGK
EAGVIYLLDGEIKYSKGAFIPKGAEILGGWAKVYRKDRTYPTETE
VSFEEYDNSKIRARVKELTQQGKDVTYPVMNSYGKPIGENNWDTM
PCVMIRKVALVSAYREAFPAELGASYEADEIQLDNTPKDITPQEN
REDVIARKMAQIEQFNKEQAHTDPEPTQTEEPIQGELLDGELEY
UPI0009F5E532
(SEQ ID NO: 247)
RTDGTKEAGAAATAPTEGKAPAKAHKPADTIGAMIEKLKPQIERA
LPKHVTPDRMARMALTAIRNNPKLGQAEAVSLMGSIIQASQLGLE
PNTPLGQCYIIPYNSKNGMQAQFQMGYKGIVDLAHRSGQYRQLTA
HPVDEADEFRYSYGLNPDLVHVPAEKPSGKITHYYAVYHLTNGGF
DFRVWSREKVEAHAKQYSKSFSSGPWQTNFDQMACKTVMIDLLRY
APKSVEIAKATSADNRTHTINPEDPDLNIDTIDGDFELEGEER
UPI0009F8F604
(SEQ ID NO: 248)
EALLLRRWQMGNLTKTTGFALAPQNLEQAMQLATMICNSQLAPNN
YKGKPEDTLVAMMMGHELGLNPLQSIQNIAVINGRPSIYGDALLA
LVQNSPAFGGIQESFDEDTMTATCTVWRKGGEKHTQHYSKDDADT
AGLWGKQGPWKQHPKRMLAMRARGFAVRNQFADALAGLVTREEAE
DMEKEINPTPAPQAQSKRIGQKQSRTQYSESDFNENFPKWKAAVE
SGKKTSEQIISMVSTKGDLTQGMIEAIESIEAGEPA
UPI000A08A794
(SEQ ID NO: 249)
GHLVSKTEQDYIKQHYAKGATDQEFEHFIGVCRARGLNPAANQIY
FVKYRSKDGPAKPAFILSIDSLRLIAHRTGDYAGCSEPIFTDGGK
ACTVTVRRNLKSGETGNFSGMAFYDEQVQQKNGRPTSFWQSKPRT
MLEKCAEAKALRKAFPQDLGQFYIREEMPPQYDEPIQVHKPKALE
EPRFSKSDLSRRKGLNRKLSALGVDPSRFDEVATFLDGTPDRELG
QKLKLWLKEAGYGVNQ
UPI000B36BD3F
(SEQ ID NO: 250)
TDVKQELERKVGKQDSTAVRLTKNMSIPDMIKALEPEIRRALPAV
LTPERFLRMALSAVNNTPKLAECTPMSFIAAMMNAAQLGLEPNTP
LGQAYLIPYKNKNQLECQFQIGYKGIIDLAYRTGQMQMIQAHAVH
EFDDFEYEYGLNPKLIHRPGDGNRGEITYFYGLFKLVNGGFGFEV
MNREAMEAFAQQYSQSYGSQYSPWVKNFEDMAKKTVIKKALKYGP
VKAEFQKAISMDETIKTEIAVDMTEVQNEESE
UPI000B38B374
(SEQ ID NO: 251)
ENEVMTQDQAYEVASPFGSSENFQKLFDIGKMFASSSLVPDRYRG
KPMDCTIAVDMANRMGVSPMMVMQQLYVVKGNPQWSGQACMSLIR
GSSEYKNVRPVYTGKKGEDSWGCYIEAEKKKTGEIVKGTEVTIAM
AKAEGWYSKKDKYGNETSKWQTMPELMLAYRAAAFFARVYIPNAL
MGCAVEGEAEDIMKRAITAEDPFKEDAK
UPI000B49B5D9
(SEQ ID NO: 252)
TLQAVCPTQDKAVESQLDQTKFELIKRTICKGTTDDEFQLFIHAC
KRTGLDPFMRQIFAVKRWDSAERREVMTIQTGIDGYRLIADRTGR
YAPGRDAEFGYDAHGGLRWAKAYVKKMTPDGHWHEISATAFWTEY
VQTTKDGRPTVFWMKKGHVMLSKCAEALALRKTFPAELSGIYTQE
EMAQTMSLPDTKGDSQTIGSDKAYEIERSIDNDPEFKTQLLTRLQ
RAFGCKSFSDLPQDQFKNVKKVIENHQIKEKIA
UPI000B4BEFE6/WP_088258624.1 Bet
[Fimbriiglobus ruber]
(SEQ ID NO: 253)
TDIAHRSYSAPQLSLIRRTVAKDTNQDEFDLFIEICKQQGLDPFK
KQIFAQVYNKDKADKRQIVIVTSIDGYRAKAQRCGDYRPAEEETR
FEADAALKIRRQPMGSFVRSCGLQVRPGQGVVSRVGEARWDEFAP
LDDAEFDWVDTGETWPDTGKPKKKKVAKSAKKTLKEGNWKNMPHV
MLGKCAEAQALGGGGRKRSAACTSKRRWTRSTWT
UPI000B5661AA
(SEQ ID NO: 254)
TASKQTDIFSFVSGGEDITITLADIKNYFCANATDQECVLFGQLC
KANGLNPWLKEAYLIKYDKNAPAAMVTGKDAYMKRANEHPAFDGY
EAGVKVYLPDVGQVEYREGTAYYEDLGEQLIGGYAKVYRKDRSRP
YYEEVPLKEYDTKQSKWKTSPATMIRKVALVHALREAFPTNIQGM
YDADETPYAADYEGSFREMDDPTPAPSMRGRIAPAPVADPLEDLE
ADVIEAGDVE
UPI000B94B1D1
(SEQ ID NO: 255)
ADLTKTANGADLAAAIGGKQAETGRATAFDLVKSMEAEFAKALPR
HVPVEQFMRTAVTELRQNADLQRSTSESLLGAFLTAARLGLEVGG
PMGEFYLTPRFAKLPGQDQKAWQVVPIVGYRGLVKLARNAGVGAV
KAWVVYEGDHFVEGANSERGPFFDFHPVPGDPAGRKEVGVLAVAR
LSGGDVQHTYLTIEQVEKRKARGSAGDKGPWATDRAAMIRKSGIR
ALAGELPQSTLLALARVVDEEVQTYVPGSLVDVGTGELEA
UPI000BD04ECE
(SEQ ID NO: 256)
NTELETMNNVYDNLQSVIMQQGIAALLPAQVTPEQFTRTAATALI
ENVDLQNADKQSLVLALTRCAKDGLMPDGREAALVVRSTKVNKQF
VKKAVYMPMVDGVIKRARQSGQVANIIAKVVYSQDEFEYVIDENG
EHLTHRPAFVDGDDIVKVYAFAKLNSGELVVEVMSRAGVEKIRDT
VQSAKYDSSPWVKWFDRMALKTVIHRLARRLPCASELFSLFEVYE
DANSTEKTLRMAPASFKRLSIN
WP_032686941.1 RecT
[Raoultella planticola]
(SEQ ID NO: 257)
TKQPPIAKADLQKTQGTRVSSPKGNNDVISFINQPSMKEQLAAAL
PRHMTAERMIRIATTEIRKVPALASCDTMSFVSAIVQCSQLGLEP
GGALGHAYLLPFGNKNEKSGKKNVQLIIGYRGMIDLARRSGQIAS
LSARVVREGDEFSYEFGLEEKLTHRPGENEDAPVTHVYAVARLKD
GGTQFEVLTSKQIELVRSQSKAANSGPWVTHWEEMAKKTAIRRLF
KYLPVSIEIQRAVSIDEKEALTIDPADTSVLTGEYSVINSESEE
WP_069728515.1 RecT
[Pantoea brenneri]
(SEQ ID NO: 258)
SNQPPIASADLQKTQQSKQVANKTPEQTLVGFMNQPAMKSQLAAA
LPRHMTADRMIRIVTTEIRKTPQLAQCDQSSFIGAVVQCSQLGLE
PGSALGHAYLLPFGNGRSKSGQSNVQLIIGYRGMIDLARRSGQIV
SLSARVVRADDEFSFEYGLDENLVHRPGENEDAPITHVYAVARLK
DGGTQFEVMTVKQVEKVKAQSKASSNGPWVTHWEEMAKKTVIRRL
FKYLPVSIEMQKAVVLDEKAESDVDQDNASVLSAEYSVLESGDEA
TN
WP_045958294.1 RecT
[Xenorhabdus poinarii]
(SEQ ID NO: 259)
TNTPPLAQADLQKAQPQTKVAATKDQALIQFINKPSMKAQLAAAL
PRHMAPDRMIRIVTTEIRKTPALANCDMQSFVGAVVQCSQLGLEP
GSALGHAYLLPFGNGKSKTGQSNVQLIIGYRGMIDLARRSGQIVS
ISARTVRDGDQFHYEYGLNENLTHIPGENEDAPITHVYAVARLQD
GGVQFEVMTRKQVEKVREKSSAGNNGPWVTHWEEMAKKTVIRRLF
KYLPVSIEMQKAVILDEKADANIDQDNAAIFEGEFEEVGNDG
WP_102086779.1 RecT
[Proteus mirabilis]
(SEQ ID NO: 260)
SNPPLAQADLQKTQGTEVKTKTKDQQLIHFINQPSMKTQLAAALP
RHMTPDRMIRIVTTEIRKTPALANCDMQSFIGAVVQCSQLGLEPG
NALGHAYLLPFGNGKAKSGQSNVQLIIGYRGMIDLARRSNQIISI
SARTVRQGDSFHFEYGLNENLTHVPGENEDSPITHVYAVARLKDG
GVQFEVMTHNQIEKVRASSKAGQNGPWVSHWEEMAKKTVIRRLFK
YLPVSIEMQKAVILDEKAEANVDQENASVFEGEFEEVGSNGN
WP_109615067.1 RecT
[Edwardsiella piscicida]
(SEQ ID NO: 261)
TNNQQPPIATADLQKAQSQAPAVKPDQKLINFINQPSMKGQIAAA
LPRHMAPDRMIRIITTEIRKTPALATCDMQSFIGSVVQCSQLGLE
PGGALGHAYLLPFGNGKAKSGQSNVQLIIGYRGMIDLARRSGQIV
SISARTVRDGDQFHYEYGLDETLKHVPGDNESSPITHVYAVAKLK
DGGVQFEVMTFNQIEKVRGQSKAGNNGPWQTHWEEMAKKTVIRRL
FKYLPVSIEMQKAVILDEKAEANIDQENASVISAEFSVVED
WP_124537594.1 RecT
[Morganella morganii]
(SEQ ID NO: 262)
SNPPIAQADLQKAQGTAVKEKTKDQQLIQFINQPGMKAQLAAALP
RHITPDRMIRIVTTEIRKTPSLATCDMQSFIGAVVQCSQLGLEPG
NALGHAYLLPFGNGKAASGQSNVQLIIGYRGMIDLARRSGQIISI
SARTVREGDSFHFEYGLNEDLTHVPGENDSGPITHVYAVARLKEG
GVQFEVMSFSQIEKVRDSSKAGKNGPWVSHWEEMAKKTVIRRLFK
YLPVSIEMQRAVILDEKAEANVDQEHASIFEGEYETVSPE
WP_006657622.1 RecT
[Providencia alcalifaciens]
(SEQ ID NO: 263)
STPPLAKSDLQKTQGTEVKIKTNEQKLVEFINQPGMKAQLAAALP
KHITSDRMIRIVSTEIRKTPSLANCDIQSFIGAVVQCSQLGLEPG
NALGHAYLLPFGNGKSDNGQQNVQLIIGYRGMIDLARRSGQIISI
SARTVRQGDNFHFEYGLNENLTHIPEGNEDSPITHVYAVARLKDG
GVQFEVMTYNQIEKVRNLSKAGKNGPWVTHWEEMAKKTVIRRLFK
YLPVSIEMQKAVILDEKAEANIEQEHSAIFEAEFEEVDSNGN
WP_109401438.1 RecT
[Proteus terrae]
(SEQ ID NO: 264)
SNPPLAQADLQKTQGTEVREKTKDQMLVEFINKPNMKAQLAAALP
RHMAPDRMIRIVTTEIRKTPELANCDMQSFVGAVVQCSQLGLEPG
NALGHAYILPFEKKRKQGNQWVTVRTDAQLIIGYRGMIDLARRSG
QIVSISARTVRQGDSFHFEYGLNENLTHVPGENEDSPITHVYAVA
RLKDGGVQFEVMTHNQIEKVRTSSKAGQNGPWVSHWEEMAKKTVI
RRLFKYLPVSIEMQKAVILDEKAEANVDQENSSVFEGEFEEVGQG
A
WP_115149784.1 RecT
[Plesiomonas shigelloides]
(SEQ ID NO: 265)
SNQRPPIATADLQKAQSQPPAAKPEQNLINFINQPSMKSQIAAAL
PRHMAPERMIRIITTEIRKTPKLATCDVQSFIGAVVQCSQLGLEP
GGGLGHAYLLPFGNGKAESGKPNVQLIIGYRGMIDLARRSGQIVS
ISSRIVREGDQFHYEYGLNETLKHVPGDNESAPITHVYAVAKLKD
GGTQFEVMSFNEIEKIRGQSKAGNDGPWIKHWEEMAKKTVIRRLF
KYLPVSIEMQRAVILDEKAEADIEQDNASIIGAEYSVVENAA
WP_034910107.1 RecT
[Gilliamella apicola]
(SEQ ID NO: 266)
SEQNQPPIAKSDLEKTQLTNQDKKPATLAELVNSPKIKNQLAMAL
PKHMNPDRMARIVTTEIRKTPALADSNIQSFLGAVVQCSQLGLEP
GGALGHAYLLPFGNGKAKDGKSNVQLIIGYRGMIDLARRSGQIIS
ISARTVREGDDFHYEYGLNEDLKHTPKADESAPITYVYAVARLKD
GGSQFEVMTFNQIESVRKQSKAGDKGPWITHWEEMAKKTVIRRLF
KYLPVSIEIQQAVILDEKAEAGISQDNEMILDADFSVVEA
WP_016979878.1 RecT
[Pseudomonas fluorescens]
(SEQ ID NO: 267)
NSTAETATPFSSQDLEKTQPTKAQSKTGSLASLLASPKMKSQFAA
ALPKHMTPERMARIVTTEIRKNPELVKCEQHSFLGAVIQCAQLGL
EPGNTLGHAYILPYGKQAQLIIGYRGMIDLARRSGQIISISARTV
REGDYFEYEFGLDENLIHRPVETTQPGAVTHVYAVARLKDGGRQF
EVMSRAQIEEVRVQSKAAKSGPWVTHWEEMAKKTVIRRVFKYLPV
SVEIQRAVMLDEKAEAGVCQENECVFDGDFEVITDTEE
WP_080977968.1 RecT
[Pseudomonas stutzeri]
(SEQ ID NO: 268)
STENVAPFSQKDMQQATGQQVKPRSPADSLAAMLASPKMKAQFAA
ALPKHMTAERMARIVTTEIRKTPALVKCDQHSFLGSVIQCAQLGL
EPGNSLGHAYLLPYGNQVQLIIGYRGMIDLARRSGQIVSLSARTV
REHDEFDYQLGLHEDLTHKPFEGEHAGEITHVYAVARLQGGGVQF
EVMSKAQVEAVRAQSKAGKSGPWVSHWEEMAKKTVIRRLFKYLPV
SVEIQRAVTLDEAAEAGLPQGNEYVFDGDFEVVNDASGAQQ
KXJ39364.1 AXA67_02205
[Methylothermaceae bacteria B42]
(SEQ ID NO: 269)
ATSLKRAVTGDGKPATVQQLLTNPKIKSQIALALPQHLTPERLTR
IVLTEIRRTPALAKCKPESLLAAVMQCAQLGLEPGGSLGHAWLMP
FKNEVQFIIGYRGMIDLARRSGQVLSIEARGVYESDTFHVSFGLE
PDLTHQPDWDPADRGKLAFVYAVARLKDGGFQFDVMSRAEVEKIR
AQSPAGKSGPWVTHFEEMAKKTVIRRLFKYLPVSVEMARAVGLDE
AAERGEQSDAIDADCVIESEEEATPEEKGDSAA
WP_106478153.1 RecT
[Halomonadaceae bacterium R4HLG17]
(SEQ ID NO: 270)
SEVATQDTLGKELQQHSGQQKKPMPTTIQGMLKDPRFTSQIARAL
PKHITPDRITRIALTEVNKTPALGKCDPVTLFGSIIQSAQLGLEL
GGALGHAYLVPYGNQAQFIIGYRGMIDLARRSGQMVSLQAHTVHD
NDEFDFEYGLDEKLRHVPARGDRGPMVAVYSVAKLVGGGHQIEVM
WKEDVDAIRSKSKAGNSGPWRDHYEEMAKKTAIRRLFKYLPVSVE
MQKAVALDEQAEAGVQDNNVFDGEFSYGEAE
WP_129141488.1 RecT
[Halomonas coralii]
(SEQ ID NO: 271)
TDQATAEPQEDLGKQLQQHSQRKPMPTTIQGMLKDDRFTGQIARA
LPKHITPDRISRIALTEVNKTPALGKCDPMSLFGSIIQSAQLGLE
LGGALGHAYLVPYKDQAQFIIGYRGMIDLARRSGQMVSLQAHTVH
ENDDFEFEYGLDEKLRHVPARGQRGPMIAVYAVAKLTGGGHQIEV
MWKEDVDAIRQQSKAGNSGPWRDHYEEMAKKTAIRRLFKYLPVSV
EMQKAVSLDEQAEAGVQDNNVEDGEFSYQEPE
WP_084261900.1 RecT
[Zymobacter palmae]
(SEQ ID NO: 272)
TNTVQQQAPQQDQLAQQLQQASGNTPQKKPMPSTIQGMLKDDRFK
TQIARALPKHVTPERIMRIALTEINKTPKLKECDPIGLFGSIVQS
AQLGLELGGALGHAYLVPYGKQAQFIIGYRGMIDLARRSGQMVSL
QAHTVHENDEFNFEYGLNENLRHVPARGERGPMIAVYAVAKLVGG
GHQIEVMWKEDVDAVRKSSKAGGSGPWRDHYEEMAKKTAIRRLFK
YLPVSVEMQRAVSLDEQAEEGVQDNNVFDGDYTVAEH
WP_020007369.1 RecT
[Salinicoccus albus]
(SEQ ID NO: 273)
STNESLKNQVATNQKNEVSNGNKPKTIGDYIDQMAPAMAQALPKH
MSVERMTRMATTVIRTTPQLKEADVASLLGAVMQSAQLGLEPGPM
GHCYFLPFKNNKKGTTEVTFIIGYKGMIDLARRSGHISTIYAHAV
YENDEFEYELGLHADLKHKPSEDERGAFKGAYAVAHFKDGGYQFE
YMPKSDIDKRRSRSKAGNSNYSPWATDYEEMAKKTVIRHMWKYLP
VSVEMQQAVAHDEGTGKDIKDVTPDEDSFVDMPEYIADVPAEGEGE
WP_131521405.1 RecT
[unclassified Lysinibacillus]
(SEQ ID NO: 274)
ATTTDLKAQMQQAPATQQKPKTIDDYLKQMAPAMAQALPKHMDVD
RLMRLAMTTIRTTPALKDADVSSLLGAVMQAAQLGLEPGLMGHCY
LLPFKNNKKGITEVQFIIGYKGMIDLARRSGHIQSIYAHAVYQKD
EFEYELGLDPKLKHKPCMDEDKGNFVGAYAVAHFKDGGYQFEFMS
KAEIEKRKGRSKAANSTYSPWATDYEEMAKKTVVRHMWKYLPISV
EMQQQVAYDEGTAPKREMKDITPETEFFVDAPEIEVEVVNE
WP_132769795.1 RecT
[Tepidibacillus fermentans]
(SEQ ID NO: 275)
ATNEKVKTQLANRANGQAPTPTPEQTIAAYMKKMAPRFAEVLPKH
MDIDRMTRIALTTIRTNPKLLEASVPSLLGAIMQAAQLGLEPGLV
GHCYLVPFKNGKTGQTEVQFIIGYKGMIDLARRSGNIESIYAHAV
YENDTFEYEYGLHPKLVHKPAMTDRGEFIGAYAVAHFKDGGYQFE
FMPKEEIEKRRNRSKTANGGPWVTDYEEMAKKTVVRHMWKYLPIS
IEIQQAAAQDEVIRKDVTSEPEFVDDVIDISTEIEEQSVEVEGEE
AQ
WP_120191052.1 RecT
[Ammoniphilus oxalaticus]
(SEQ ID NO: 276)
STKATSNELKNQLANRQGNNAATNNNPANTIAAYLKRMAPEIEKA
LPAHMDADRLARIALTTIRTTPKLLECTIPSLMGAVMQSAQLGLE
PGLIGHCYIIPYGKEATFIIGYKGMIDLARRSGNIESIYAHAVYK
NDEFEYEYGLKPNLVHKPAMSDQGDFIGAYAVAHFKDGGYQFEFM
PKEEIDKRRNRSAASKGGPWVTDYEEMAKKTVVRHMWKYLPISIE
IQQAATQDEVVRKDITEDPMPVDVLDIPFEASDAEETSEEGEINF
D
WP_066790810.1 RecT
[Rummeliibacillus stabekisii]
(SEQ ID NO: 277)
ATTTELKEQMKQQAPAQTKKPKTIEDYMKQMAPAMAEALPKHMSV
DRLTRLAMTTIRTTPALRQADVSSLLGAVMQAAQLGLEPGLLGQC
YLLPFKNKKKAITEVQFIIGYKGMIDLARRSGHIQSIYSHAVFEN
DVFEYELGLEPKLKHTPTMSTDKGAFIGAYAVAHFKDGGHQFEFM
SKADIEKRKGRSKAANSDYSPWLTDYEEMAKKTVIRHMWKYLPIS
VEMQEQVAYDEGVGRSIKDVTPEEDVFVQAPDEILEAEATEA
WP_098408280.1 RecT
[Bacillus]
(multispecies)
(SEQ ID NO: 278)
TQAEKLKNDIAKQEQKNEVAQDDKPKTILDVMMQHKESFEMALPK
HLDADRLIRLAVTEFRKNPMLKECTPESLLGAVMQAAQVGLEPDA
LGSAYLVPYYNKNKNVKEVQLQIGYKGLIELVRRSGQVTSIVANE
VYENDEFDFEYGINEKLYHKPTMDADRGKLKCFYAYARFKDGGHA
FTVMSVEQINQIRDKFSKSQKNGKHFGPWADHYESMAKKTVIKQL
VKYMPISVEIQNQITRDETVHSSFKEEPKPIYAFEESPDIIDAPI
EN
WP_047150996.1 RecT
[Aneurinibacillus tyrosinisolvens]
(SEQ ID NO: 279)
SDLKEKLEKRANETEAAPPSPAQTIAAYLKRMEPEIARALPKHMD
VERLTRIALTTIRTNPRLLECTVPSLLGAVMQAAQLGLEPGLLGQ
CYIIPHGREATFIIGYKGMIDLARRSGNIKSIYAHDVRENDEFEY
EYGLHPFLKHRPAMTDRGKFIGVYAVAHENDGGYQFEFMPYEEIE
RRKLRSRSYKNGPWVTDYEEMAKKTVIRHMFKYLPLSVEIMRSAA
QDETVRPDLTSDPVSIYERPIEGKIITAEDVQPEEIPNVPDAEQG
DV
WP_018705791.1 RecT
[Siminovitchia fordii]
(SEQ ID NO: 280)
ATNQDIKNQLANKANGNKPASPANTIAAYLKKMGPEIEKALPKHM
DADRLARIALTTIRTTPKLLECNISSLMGAVMQSAQLGLEPGLIG
HCYIIPYGKEATFIIGYKGMIDLARRSGQIQNIYAHAVFENDEFD
YALGLHPKLEHKPAGSNRGEIIGAYAVAHFKDGGYQFEYMAKEDI
EKRKSRSAAARSKHSPWATDYEEMAKKTVIRHMWKYLPISVEIQQ
QAIQDEVVRKDVTSEPEFIDMEDMPEVEEGQSEESEQVEAPFD
WP_035430909.1 RecT
[Bacillus] sp. UNC322MFChir4.1
(SEQ ID NO: 281)
ATNKDVKNQLANRKENKPATPEQKVEAYMTAMAPRFAEVLPKHMS
MDRMSRIALTTIRTNPKLLECSVPSLMGAVMQAVQLGLEPGLLGH
CYILPYKSEATFIIGYKGMIDLARRSGHIQSIYAHAVYENDEFDY
ELGLHPKLTHKPSFGERGEFIGAYAVAHFKDGGHQMEFMPKSEIE
KRRSRSASGNSSYSPWKSDYEEMAKKTVVRYMFKYLPISIEVQSQ
AQHDEVVRKDITEEPEFIEMDSIEVAEASEGDGQKEFVIEE
RDC50983.1 RecT
[Acinetobacter] sp. RIT592
(SEQ ID NO: 282)
ADLKNKLANKAAGTVTKTSPNAGMKQLMKSMSKEIEAALPSHMSS
ERFQRVALTAFGNNPKLMNCDPMSFIAAMMDSAQLGLEPNTPLGQ
AYLIPYGTKVQFQVGYKGLLELALRSGKIKTLYAHEVRENDTFEV
KYGLHQDLIHEPVLKGNRGEVIGYYAVYHLDTGGHSFVFMTKDEV
LEHAKGKSKTFNNGPWQTDFDAMAKKTVIKQLLKYAPLSIEMQKA
VSSDETVKSKIDEDMSLVVDESDSIEANFEIKEDEDGQLDVYVK
WP_150051132.1 RecT
[Methylomonas rhizoryzae]
(SEQ ID NO: 283)
SELLSALNAPETQKPQTLPAMLKQHQPRFKAIAPRDVDVTRFSAA
LMADVRSNQKLAECNPMTVLGAFIRSTQLGLEPGSQLGQAYFVPF
KGECQLVIGYRGMIELAYRSGKVASISARTVYENDVFEWELGTDE
RITHKPATGDRGALVAVYAMAKLTTGGIHFEVLDLAEIEKAKRAS
KSSSFGPWKDHFEEMAKKTAIRRLFKYLPVGTDLTRAVALDEKAE
SGSQQNDIEAETVLDGEFYPAGGGNDG
WP_097006457.1
[Lacrimispora amygdalina]
(SEQ ID NO: 284)
AVDVKNELERKASGQNSQVKLTKSMTIADMVKALEPEIKRALPAV
LTPERFTRMALSAINNTPELAGCTPMSFIAAMMNAAQLGLEPNTP
LGQAYLIPYKNKGVLECQFQIGYKGMIDLAYRTGQIQVIQGQAVR
EFDYFEYQYGLDPKLVHRPGEEERGEITFIYGLFRLSNGGYGFEV
SNKADMDAFAAKYSKSFGSKYSPWTENYEDMAKKTVIKRALKYAP
VSVDFQKAMSMDETIKTEISVDMSEIRNECPEISENGEAA
WP_087225255.1 RecT
[Lachnoclostridium] sp. An14
(SEQ ID NO: 285)
TDVKQELERKVGKQDSTAVRLTKNMSIPDMIKALEPEIRRALPAV
LTPERFLRMALSAVNNTPKLAECTPMSFIAAMMNAAQLGLEPNTP
LGQAYLIPYKNKNQLECQFQIGYKGIIDLAYRTGQMQMIQAHAVH
EFDDFEYEYGLNPKLIHRPGDGNRGEITYFYGLFKLVNGGFGFEV
MNREAMEAFAQQYSQSYGSQYSPWVKNFEDMAKKTVIKKALKYGP
VKAEFQKAISMDETIKTEIAVDMTEVQNEESE
WP_002566991.1 RecT
[Enterocloster bolteae]
(SEQ ID NO: 286)
GVNVKHELEQRAAGQGASVRLTKNMTIVDMVKALEPEIRRALPAV
LTPERFTRMALSSINNTPELAECTPMSFIAALLNAAQLGLEPNTP
LGQAYLIPYKNKGKLECQFQLGYKGLIDLAYRTGQVQIIQAQVVR
EFDSFEYQYGLDSKLVHKPGEGARGEITYVYGLFKLSNGGYGFEV
SNKTEMDTFAARYSKSFGSKYSPWTEDYESMAKKTVIKRVLKYAP
ISSDFQKALSMDETIKTGIAVDMSEIRNECLPEEAGSEAA
WP_132412730.1 RecT
[Kribbella albertanoniae]
(SEQ ID NO: 287)
ATADSVREELARSKEVERTQPKASNADNVIGLINRSLPEIAKALP
GHVKPERIARIATTAVRVTPKLADCTQASFLGALLTAAQLGLEPN
TPTGEAYLLPFGRNVQLIIGYRGYIKLANQSGQVRNIMAMTVYEN
DHFDYKYGSNPFLEHTPTLGQDPGPVKCWYACATFTNGGTNFVVL
DKFKVEGYRARARSKDDGPWVTDYDAMARKTCIRRLAPYLPMSVE
LAQAMQVDEEVTAFTPGVSDPEVLATLAGVDTGTGEVQQ
WP_130067396.1 RecT
[Bacillus albus]
(SEQ ID NO: 288)
ATNEKLKNQLANRKESAPATPEQTVEAYMKKMAPKMAEVLPKHMD
MGRMSRMALTTMRTSPKLLNCTVSSLMGAVMQAVQLGLEPGLLGH
CYILPYKGEATFIIGYKGMIDLARRSGHIQSIYAHAVHENDEFDY
ELGLHPKLEHKPVHGDRGAFVGAYAVAHFKDGGYQMEFMPKSEIE
KRRKRSASANSSFSPWKSDYEEMAKKTVIRYIFKYLPISIEVQLL
AAQDEVVRKDITEEPEFIEADPIDVEQPTTEGDGQQEFSIEE
WP_087099033.1 RecT
[Bacillus cytotoxicus]
(SEQ ID NO: 289)
ATNEKIKNQLANRKANASLSPEQTVEAYMKKMAPRFAEVLPKHMD
MDRMSRIALTTIRTNPKLLECNVPSLMGAVMQAVQLGLEPGLLGH
CYILPYKGEATFIIGYKGMIDLARRSGHIQSIYAHAVYENDEFEY
ELGLNPQLKHKPSFGDRGEFIGAYAVAHFKDGGHQMEFMPKSEIE
KRRKRSASANSNYSPWKSDYEEMAKKTVVRYMFKYLPISIEVQSQ
AQHDEVVRKDITEEPQFIEADSVEVEETPTEGTNQEEFVIEE
WP_149216302.1 RecT
[Bacillus] sp. JAS24-2
(SEQ ID NO: 290)
ATNKDVKNQLANRKASAPVTTEQTVEAYMKKMGPKMAEVLPKHMD
MDRMSRIALTTIRTNPKLLECSVPSLMGAVMSAVQLGLEPGLLGH
CYILPYKSEATFIIGYKGMIDLARRSGHIQSIYAHAVYENDEFEY
ELGLHPQLKHKPSFGDRGEFIGAYAVAHFKDGGHQMEFMPKSEIE
KRRGRSASANSNYSPWKTDYEEMAKKTVVRYMFKYLPISIEVQSQ
AQQDEVVRKDITEEPEFIEVEQQTEGDGQGDFVIEGE
WP_125141636.1 RecT
[Clostridium transplantifaecale]
(SEQ ID NO: 291)
TDVKEELARKAGNTGKQEIRLNKNMSIPDMVKVLEPEIKRALPSV
LTPERFTRMALSAINNTPKLAECSPMSFIAALMNAAQLGLEPNTP
LGQAYLIPYKNKNQLECQFQIGYKGYIDLAYRTGQVQMIQAQAVH
EFDYFEYEYGLTPKLVHRPGEGERGEITYFYGLFKMINGGFGFEV
MNRAAMDAFAKQYSQSINSKYSPWNSQYEEMAKKTIIKKALKYGP
VKSDFQKAISMDESIKTELSIDMSEVRNEDLIDGEFEEAA
WP_120055566.1 RecT
[Lachnoclostridium pacaense]
(SEQ ID NO: 292)
TDVKQELEKRAGSSNQAIKLTKSMTIVDMVKALEPEIKRALPAVL
TPERFTRMALSAINSTPKLAECTPMSFIAALMNAAQLGLEPNTPL
GQAYLLPYKNKGVLECQFQIGYKGVIDLAYRTGQIQMIQAQAVRE
SDYFEYQYGLEPKLVHRPGDGARGEVTFIYGMFRLTNGGYGFEVS
NKADMDAFAEKYSKSYGSRYSPWTENYEDMAKKTVIKRALKYAPI
SSDLQKALSSDETIKTVLSVDMSEINNECQIDEVIQEDAA
WP_118246619.1 RecT
[Clostridium] sp. AM58-1XD
(SEQ ID NO: 293)
SVDVKNELEKRAAGTVNPAVKLTKNMTIVDMVRALEPEIKRALPT
ILTPERFMRMALSAINNTPELADCTPMSFIAALMNAAQLGMEPNT
PLGQAYLIPYKNKGTLECQFQIGYKGLIDLAYRTGLIQVIQAQTV
REFDSFEYQYGLDSRLTHRPGDGERGEITYIYGLFKLTNGGYGFE
VSNKADMDAFAEKYSKSFGSRFSPWKENYEDMAKKTVIKRALKYA
PVSSDFQKALSMDETIKSELSIDMSEIRNECQVEASGQEGAA
WP_025114396.1 RecT
[Lysinibacillus fusiformis]
(SEQ ID NO: 294)
ATTNELKAKSQNQVQQNVTPEQSLNTLLKRMGPQIQRALPKHMDA
DRIARIALTAVRATPKLLECDQMSFVAALMQSAQLGVEPNTGLGQ
AYLIPYGKQVQFQLGYKGLIDLAVRSGQYKAIYAHEVYKEDEFSF
AYGLHKDLVHVPSTNPEGEPIGYYAVYHLKNGGYDFVYWTRERID
KHAHEFSQAVKKGWTSPWKTNYDAMAKKTVLKEVLKYAPKSIELQ
KVVEADETIKTEVSEDMSDVIDVTDYSVIEDESAQEELIIEQ
WP_083048409.1 RecT
[Marispirochaeta aestuarii]
(SEQ ID NO: 295)
RTDGTKEAGAAATAPTEGKAPAKAHKPADTIGAMIEKLKPQIERA
LPKHVTPDRMARMALTAIRNNPKLGQAEAVSLMGSIIQASQLGLE
PNTPLGQCYIIPYNSKNGMQAQFQMGYKGIVDLAHRSGQYRQLTA
HPVDEADEFRYSYGLNPDLVHVPAEKPSGKITHYYAVYHLTNGGF
DFRVWSREKVEAHAKQYSKSFSSGPWQTNFDQMACKTVMIDLLRY
APKSVEIAKATSADNRTHTINPEDPDLNIDTIDGDFELEGEER
WP_099424140.1 RecT
[Solibacillus] sp. R5-41
(SEQ ID NO: 296)
ATSNELKKQAQGQVTAKPTTPEGSLNALLKKMGPEIQRALPKHMD
ADRIARIALTAVRTTPKLLECDQLSFVAALMQSAQLGVEPNTGLG
QAYLIPYGGKVQFQLGYKGLIDLAVRSGQYKAIYAHEVYADDEFS
FAYGLHKDLVHVPSANPSGDPIGYYAVYHLKNGGYDFVYWTRERI
DIHSKAFSQAVQKGWTSPWKTNYDAMAKKTVLKEVLKYAPKSIEM
QKVVEADETIKNEVAPDMSNVIDVTDYSILEDPQDVTDAQ
WP_076065282.1 RecT
[Viridibacillus] sp. FSL H8-0123
(SEQ ID NO: 297)
ATNNALKEQMKQAPSKEVKPEQSLNTLLKRMGPEIQRALPKHMDA
DRIARIALTAVRNTPKLLDCDQMSFVAALMQSAQLGVEPNTGLGQ
AYLIPYGKQVQFQLGYKGLIDLAVRSGQYKAIYAHEVYEDDEFSF
AYGLHKDLVHVPAPNPTGEPIGYYAVYHLQNGGYDFVYWTRERID
QHAHKFSMAVQKGWTSPWKTNFDAMAKKTVLKEVLKYAPKSIEMQ
KVVDADETVKTDVSDDMSNVIDVTDYTVMDQEQETIQEPTK
WP_024292388.1 RecT
[Lacrimispora indolis]
(SEQ ID NO: 298)
SDVKQELEKRAAGGGGQSQSVRLTKNMTIVDMVKALEPEIKRALP
SILTPERFTRMALSAINNTPKLGECTPMSFIAALMNAAQLGLEPN
TPLGQAYLIPYKNKGVLECQFQLGYRGLIDLAYRNERMQSVEAQV
VYENDEFSYELGLHPSLIHRPSFDEPGEIRAFYAIFRLDNGGFRF
EVMSKSYVDAYAARYSKAFTSDFSPWKSNYEGMAKKTVIKQLLKY
APMKSEFQKAVTMDETIKTELSVDMSEVSNQEVIDRELTEQVA
WP_009524931.1 RecT
[Peptoanaerobacter stomatis]
(SEQ ID NO: 299)
GAKELIQKKQENKQISPTSNMNMLLQSMAGAIKKALPAQINSERF
QRVALTAFSSNQKLQQCDPISFLAAMMQSAQLGLEPNTPLGQAYL
IPYGKQVQFQVGYKGLLELAQRSGQFKSIYSHEVRENDEFEMEYG
LNQKLVHKPNLKQERGEVIGYYACYHLTNGGESMFFMTKDEIINF
GKSKSKTFNNGPWQTDFDAMAKKTVLKQLLKYAPLSIESQKFMSM
DETVKSDISANMDEINNDTVDFEVDIQTGEVINDIVVENTNEDEA
N
WP_015358111.1 RecT
[Thermoclostridium stercorarium]
(SEQ ID NO: 300)
TTVNQTELKNKLAEKAKTPAKTGNTVFDLIRKMEPEIKRALPKQI
SPERFARIAMTAVRNTPKLQACEPISFIAALMQSAQLGLEPNTPL
GQAYLIPYGKEVQFQLGYQGMLTLAYRTGEYQSIYAMPVYANDEF
EYEYGLNEKLVHKPAPDPEGEPIYYYAVYKLKNGGHGFVVMSRQQ
IERHRDKYSPSAKQGKFSPWNTDFDSMAKKTVLKQLLKYAPKSVE
FATQIAQDETIKTEIAEDMTEVQGIEVEYEATDDQENQENQEQED
WP_002595146.1 RecT
[Enterocloster clostridioformis]
(SEQ ID NO: 301)
GIDVKHELEKRAAGQDKPVKLTRNMTIADMVKALEPEIKRALPAI
LTPERFTRMALSAVNNTPELANCTPMSFIAALMNAAQLGLEPNTP
LGQAYLIPYKNKGTLECQFQLGYKGLIDLAYRTGQIQIIQAQAVR
EFDYFEYQYGLDSRLVHKPGNEERGQITFIYGLFKLSNGGYGFEV
SNKAEMDAFAAKYSKSFGSKYSPWTEDYESMAKKTVIKRALKYAP
VSSDFQKALSLDETVKSEIAVDMSEIRNDCIPADMGTEAA
WP_100306418.1 RecT
[Lacrimispora celerecrescens]
(SEQ ID NO: 302)
SDVKQELEKRAAGGGSQSQSVKLTKNMTIVDMVKALEPEIKRALP
SILTPERFTRMALSAINNTPKLGECTPMSFIAALMNAAQLGLEPN
TPLGQAYLIPYKNKGVLECQFQLGYRGLIDLAYRNDRMQSIEAQV
VYENDEFSYELGLHPSLTHRPSFDEPGEIRAFYAIFRLDNGGFRF
EVMSKSYVDAYATKYSKAFTSDFSPWKNNYEGMAKKTVIKQLLKY
APIKSDFQKAITLDETVKTQLSIDMSEIRNECLPDTSENSEVA
WP_071062796.1 RecT
[Andreesenia angusta]
(SEQ ID NO: 303)
SNLKNQLANKAGGTATKKQPQTMQDWIKVMEPQIKKALPSVITAE
RFTRMALTAISTNPKLAECTPESFMGALMNAAQLGLEPNTPLGQA
YLIPYGKSVQFQVGYKGLMELAQRSGQFKSIYAHTVYENDEFEVE
YGLTQNIVHKPNFDDRGKPIGFYAVYKLTNGGENFVFMTQREVEE
FGKAKSKTFNNGPWKTDFEAMAKKTVLKQLLKYAPIKVEFQREIA
QDATIKTEIAEDMTEVPEEMVEAEYEVVEQNTMAEDADLKGTPFE
TK
SFO83314.1 RecT
[Amycolatopsis arida]
(SEQ ID NO: 304)
HGTALNPERFTRVALTVIRQSADLQRCRPESLLGALMTSAQLGLE
PGPLGEAYLVPYGDQVTFIPGYRGLIKLAWQSGQLRHISARVVHE
GDRESYSYGLHPDLIHQPTRGDRGPITDVYAAATLIDGGVEFEVL
DVATVETIRARSRAGRKGPWVTDWEAMARKTAIRQLAKWLPMATV
MSRAIAAEGTVRTDLDADALDDLTADPGPEVLDADPAWDGPEPPG
DQARNQEPTTQGDA
WP_110092637.1 RecT
[Corynebacterium striatum]
(SEQ ID NO: 305)
GTNLEQRMAANNAPAKQNRPVTLADQIRSMESQFQLAMPKGMEAQ
QLVRDALTCLRQTPKLAECTPQSVLGGLMTCSQLGLRPGVLGHAY
LLPFWDRKQGGMVAQLVVGYRGLVELAHRSGQIQSLIARTVYEND
HFDVDYGLDDKLVHKPCMNGPKGNPIAYYAVAKFTTGGHSFIVMS
KDEMLAYRDEFAKAKNKQGEVFGPWADNFDAMAHKTCVRQLAKWM
PSSTDLDRGIAADETVRVDLSESALDYPQHVDGEVVDSKPAEDEA
A
WP_129692339.1 RecT
[Gottfriedia acidiceleris]
(SEQ ID NO: 306)
ATPAELKNLLAAKPKGEVKLTPDQQVSSYLKAYEGTFRQIAPKHE
NTERFQRIALSEIRKNPKLLDCNLPSLMSAVLQSVKLGLEPGLFG
QAYLIPYGKEVQFQIGYKGLIELAQRSGRIAKIQAREVYEHDEFE
VSYGIDDTIIHKPKLDGDRGDVRLYYAVAWFKDGAAQFEIMSKSD
VENHRDKFSKTKNYGPWKENFDAMARKTVLKKLVNQLPMDVEFHE
AVQEDETVRKTINDEPEVIAAEYEIIDAPEVVEGNE
WP_118016648.1 RecT
[unclassified Coprococcus]
(multispecies)
(SEQ ID NO: 307)
ANNIDLKQELAEQASKVPAKKDEEVKLTKSMTIPDMVKAMMPEIK
KALPAVMTPERFTRIALSALNTTPALNQCTPMSFLAALMNAAQLG
LEPNTPLGQAYLIPYKNHGTLECQFQIGYKGLIELAYRSGQMQTI
QAQTVYENDEFAYQYGLEPVLVHRPAYSDRGEVKYFYGIFKTVNG
GYGMAVMSRAEMDLYAKTYSKAYDSSYSPWKSNYEDMAKKTVIKQ
ALKYAPIKTDFQRALSFDETIKKEISLDMSTVKNELLDVA
WP_051200279.1 RecT
[Butyrivibrio] sp. FCS006
(SEQ ID NO: 308)
PYLFGGQMKEQEIKNQLAAKAVETTNPKLSKNMNIADLIKAIEPE
IKKALPTVITPERFTRIALSALNTTPKLAECSQMSFLAALMNAAQ
LGLEVNSPLGQAYLIPYNNKGKLECQFQIGYKGMLGLAYRNPEIQ
TIQAQVVYENDDFKYELGLDSKLYHKPSLSDRGKVRCYYALYKLR
NGGYGFEVMSRRDVEEYAKRYSKVTDSLYSPWANNFDSMAKKTVI
KQLLKYAPLRTDLEKAMSMDESIKTRVSVDMSEVENEETFDAEVE
V
WP_107514794.1 RecT
[Staphylococcus equorum]
(SEQ ID NO: 309)
ATNETLKQKVVERKPNGVKEQSPKTQLNHLLKKMAPEIQRALPKH
MDSDRMARIAMTAVSNTPKLLECDQMSFIAALMQASQLGVEPNTG
LGQAYLIPYAGKVQFQLSYKGLIDLATRSGQYKSIYAHEVYTNDE
FEYRYGLFKDLIHIPSQEPEGNPIGYYAVYHLKNGGYDFVYWTRE
RVDKHAKEFSQAVQKGWTSPWITNYDAMAKKTVLKEVLKYAPKSI
EMNKAVENDSTIKEEIDKDMSTVIDVTDYSEVEEQESLETGGQTS
K
WP_117624242.1 RecT
[Hungatella hathewayi]
(SEQ ID NO: 310)
RRDRNVTAVKQELEKKAAGTSQAVKLTKNMTIVDMVKALEPEIKR
ALPSILTPERFTRMALSAINNTPKLAECTPMSFIAALMNAAQLGL
EPNTPLGQAYLIPYKNKGVLECQFQLGYRGMIDLAYRNERMQSIE
AQTVYEHDEFFYELGLHPALVHRPTFEDRGEIRAFYAIFRLDNGG
YRFEVMSKSYVDAYAMRYSKAFTSEFSPWKSNYEGMAKKTVIKQL
LKYAPVKSEFQKAITLDETVKTELSVDMSEVQNEDLSETLTAESA
A
WP_118771779.1 RecT
[Roseburia intestinalis]
(SEQ ID NO: 311)
GDIRSELAKKAEQTQGNTKLTKSMSIADLIKAMEPEIQKALPSVI
TPERFTRMALSALNTTPKLQECTPMSFLAALMNAAQLGLEPNTPL
GQAYLIPYKNKNVLECQFQLGYRGMIDLAYRNGHMQSIEAQAVYE
NDVFSYALGLHPELVHKPTLEEKGALKAFYAIFRLDNGGFRFEVM
GKTYIDWYANRYSKAFTSEFSPWKSNYEGMAKKTVIKQLLKYAPL
KTEFQRALSTDETIKNSLNVDMGEVLSEDIIDMPCEEVA
WP_107378794.1 RecT
[Staphylococcus chromogenes]
(SEQ ID NO: 312)
ANAKEFKKQMNSKNEVAETNNAPQKAKGPRQQVSDLLDRMAPEIQ
KALPNNMSAERMARIAMTAVSSNPKLLECDPKSFIGALMQASQIG
LEPNTALGQAYLIPYGNQVQLQLSYLGLIELATRTGQYKAIYAHE
VYKDDEFSYEYGLYKNLIHKPVDDPNGEPIGYYAVYHLMNGGYDF
AYWTRKKVEAHAQQYSKAVQQGWNSPWKSDFNAMAKKTVLKDLLK
YAPKAIEVSQAIGSDSKVSEINDEGEIIDVTDYSQEEEK
WP_094369469.1 RecT
[Romboutsia weinsteinii]
(SEQ ID NO: 313)
TNLKNTLKNKEAKGNNLAINPSYAMKQLMIKMKGEITSALPKELC
SERFQRVALTAFNSNPKLQNCAPMTFIAAMMQSDQLGLEPNTPLG
QAYLIPYKVKGIDKVQFQIGYKGLLELAHRSGRLKTLYAHEVREN
DEFDIDYGLEQRLIHKPLLKGNRGEVIGYYAVYHLEHNGYSFVFM
TYDEVLEHGKKYSKSFEGGIWEKEFDSMAKKTVIKKLLKYAPLSI
EIQKAINFDESVKGSIDSDMLLVDKADESIDVEGNVLNQRGIKYG
CI
CDF42377.1
[Roseburia] sp. CAG: 182
(SEQ ID NO: 314)
DVKEELAKMAEEKPTKKLTKSMSIQDMIKVIEPEIKKALPSVLTP
ERFTRMALSAINNTPKLAECSQISFLAALMNAAQLGLEPNTPLGQ
AYLIPFQNKGKLECQFQIGYKGIIELVYRNPLIQTIQAQVVYEND
EFEYELGLNSRLFHRPALYDRGETVLFYALFKMSNGGYGFEVLSK
QDMDAYAKRYSKGISSEYSPWKSNYEEMAKKTMIKKVLKYAPIRT
DFQKAVSMDESIKKELSVDMSEVSNENIIDMEEITQEEE
WP_123609006.1 RecT
[Mobilisporobacter senegalensis]
(SEQ ID NO: 315)
KDIKSALEKKVDKQDVKLTKSMSITDMIKALEPEIKKALPSVITP
ERFTRMALSAVNNTPKLAECSQMSFLAALMNAAQLGLEPNTSLGQ
AYLIPYQNKGKLECQFQLGFKGMIDLVYRNEKVQTIQAHCVYEED
YFEYELGLDSKLAHKPALANRGKMILVYAFFKLENGGFGFEVMSK
EDIDIHALKYSKGYSSQYSPWKSNYEDMAKKTVIKKVLKYAPLKI
DFQRAISVDETVKAEISIDMSEVQNEEIIDGQCTDVGEIEEK
WP_115856892.1 RecT
[Staphylococcus felis]
(SEQ ID NO: 316)
ANANSFKEQVSNKNEVSENNNTPQQKTKGPRQQVSDLLERMAPEI
QKALPSHMSAERMARIAMTAISSNTQLLECNPRSLIGALLQASQI
GLEPNTALGQAYLIPYYNRNKGEFEAQLQLSYLGLIELATRTGQY
KAIYAHEVYKEDEFYYEYGLHKNLVHKPVDDPKSEPIGYYAVYHL
QNGGYDFSFWTRNKVELHSGQYSKAVQKGWNSPWKTDFNAMAKKT
VLKDLLKYAPKSVEVSRAVGTDSKVSEISQNGEIIDVTDYSKEEE
WP_108404827.1 RecT
[Corynebacterium liangguodongii]
(SEQ ID NO: 317)
KDLETRMAANQQPAQQRPTTLADQIRGMEQQFALAMPKGAEASQL
VRDALTALRQAPKLAQCTPQSVLGSLMTCAQLGLRPGVLGHAYLI
PFYDRRAGGLVAQLVIGYQGLVELAHRSGQIKSLIARTVYENDVF
DVDYGLEDKLVHKPYMGGDKGQPIAYYAVAKFTTGGHAFYVMSHP
EMLDYRARFAKSAERGPWVDNFEAMALKTCVRQLSKWMPKSTELA
TAIAADESVRVDLTPDAINYPEHVDGEVVDAQGTTEDTAGEGEQS
A
WP_021747387.1 RecT
[unclassified Oscillibacter]
(multispecies)
(SEQ ID NO: 318)
KEGLIQGTQSAQAAKKGPATMQDYIKKMQGEIAKALPSVLTPERF
TRITLSALSTNPKLAQTTPKSFLGAMMTAAQLGMEPNTPLGQAYL
IPFKNHGVLECQFQLGYKGLIDLAYRSGEVSTIQAQTVYENDEFE
YELGLEPKLHHVPAKGERGEPVYFYAVFRTKDGGYGFEVMSVDDV
RTHAKKYSKAYSNGPWQTNFEEMAKKTVLKKALKYAPLKTEFMRG
LTSDETIKTEISEDMYSVPDETVIEAEGYEVDGDTGEVIERPADG
Q
WP_103110615.1 RecT
[Brevibacillus reuszeri]
(SEQ ID NO: 319)
SNKLAQRAGQQTQPVKPDQQISALLKRMEPEIARALPKHLTSDRL
ARIAMTSIRQNPKLLACDQMSLLAGVMQSAQLGLEPNTPLGEAYL
IPYGKEAQFQVGYKGIISLAHRTGEYQAIYAHEVFKNDEFSYSYG
LDKTLNHKPADEPEGDPIYFYAVYRLKNGGFDFVVWSTKKIDAHA
KKYSQAYQKGWTTPWKTDFVAMAKKTVLKEVLKYAPKSAEMAKAL
VMDETVKNEISEDMSEVPGMVIDIEADAANVEETAGGGASE
WP_016998679.1 RecT
[Mammaliicoccus]
(SEQ ID NO: 320)
ATNESIKNQVASRKKNEVQNKSPKTQLNDLLIKMGPEIQRALPKH
MDADRMARIAMTAVSTTPKLLECDQMSFIGALMQASQLGVEPNTG
LGQAYLIPYGGKVQFQLSYKGLIDLATRSGQYKAIYAHEVFPNDE
FNYQYGLFKNLEHIPSQEPEGEPIGYYAVYHLKNGGYDFVYWTRE
RVDKHAKDFSQAVQKGWTSPWKTNYDAMAKKTVLKEVLKYAPKSI
EMNKAVNSDSTIKDEINEDMSSVIDITDYEEVNDQQEEKKEESK
WP_147540090.1 RecT
[Clostridiaceae bacterium]
(SEQ ID NO: 321)
SNLKKALKTNETKGNSVTVSKAYAMKQLMIKMKGEITSALPTNLS
SERFEKVALTAFNSNPKLQKCDPRTFIAAMMQSAQLGLEPNTALG
LAYLIPYEVKGINKVQFQIGYKGLLELANRSGKLKTLYAHEVREN
DEFDIDYGLEQKLIHKPLLKGNRGNVIGYYAVYHLEPSGYNFVFM
TYDEVLEHGKKYSKSFEGGVWEKEFDSMAKKTVIKKLLKYAPLSI
EMQKAIVFDESVKGSIDSDMLLVDKEDESIEGSELN
WP_019168122.1 RecT
[Staphylococcus intermedius]
(SEQ ID NO: 322)
ANANSFKEQVSKNEVQETNNEKPKGPRQQVSDLLERMAPEIQKAL
PSHMSAERMARIAMTAISSNPQLLECNPRSLIGALLQASQIGLEP
NTALGQAYLIPYYNHKKKEFEAQLQLSYLGLIELATRTGQYKAIY
AHEVYKEDEFYYEYGLHKNLVHKPVDDPNGEPVGYYAVYHLQNGG
FDFAYWTKNKIELHAGNYSKAVQKGWNSPWKTDFNAMAKKTVLKD
LLKYAPKSIEISQAVGSDSKVTEINKQGEIIDITEYGQEALEG
WP_148820236.1 RecT
[Corynebacterium urealyticum]
(SEQ ID NO: 323)
AKNLEARMQQSTNAPARADKPLSLPDQIRQMEDQFRLAMPKGAEA
TQLVRDALTCLRQTPQLAQCTPASVLGGLMTCAQLGLRPGVLGHA
YLIPFNDRRSGNSVAQLVIGYQGLVELAHRSGQIKALIARTVYEN
DHFDVDYGLEDKLVHKPHMGADKGNPVAYYAVVKFTTGGHAFYVM
SHPEMLQYRDKNAKSPKRGPWVDNFEAMAHKTCVRQLAKWMPKST
EFSQALATDESIRLDVTPDAINYPDHPAEGEVIDGEVEQDGGQQ
WP_096823857.1 RecT
[Staphylococcus nepalensis]
(SEQ ID NO: 324)
ATQNQFKNQLTQKKENNNQPQQKAVGPKQEISNLLDRMAPQIQKA
LPQHMSAERMARIAMTAVSSTPKLLECDPKSLIGALMQSSQIGLE
PNTNLGQAYLIPYGKEVQLQVSYLGMIELANRSKQYKAIYAHEVY
PEDYFEYQYGLQKDLIHKPADNPQSEPIGYYAVYHLLNGGYDFVY
WSKAKIDDHARQFSKAVQKGWQSPWKTNFNAMAKKTVLKDLLKFA
PKSIEMNNAVSSDSKAQQIDDDGNIIDVTDYSQVNDEPEQLQEGQ
WP_098170605.1 RecT
[Bacillus] sp. AFS017336
(SEQ ID NO: 325)
ATNESLKNQITNKKTGEVPLTPAQQVSSYLKAYEGTFQQIAPKHE
NTERFQRIALSEIRKNPKLLECSVPSLMSAVLQSVKLGLEPGLFG
QAYLIPYGKEVQFQIGYRGLIELSQRSGRILKIQAREVYENDEFE
VSYGIDDNIIHKPALDVDRGKVRLYYAVAWFKDGGAQFELMSISD
VEKHRDKFSKTAKFGPWKDHFDEMAKKTVLKKLVKQLPMDVEFQE
AVQEDETVRKTITDEPEILQAEFEIVDQPEISVE
WP_087290962.1 RecT
[Pseudoflavonifractor] sp. An184
(SEQ ID NO: 326)
ATEKAIQRATGRAPALENRPALQQYIKQMSGEIKKALPSVMTPER
FTRIVLSALSTNPKLAETTPQSFLGAMMTAAQLGLEPNTPLGQAY
LLPYWNSKANAYECQFQLGYKGLLDLAYRSGEISVIQAHVVYSED
QFSYSFGLKPELKHIPAGEERGEPVYVYAIFHTKDGGYGFEVCSI
DDIRAHAQRYSKSFQNGPWQTNFEEMAKKTVLKRVLKYAPLKSEF
LRGLAQDETIKQEISEDMYMVEAAYAEPDVSSAEND
WP_051264703.1 RecT
[Nakamurella lactea]
(SEQ ID NO: 327)
ASNLAARAAEQVEQQTAPNRPPTIKEQIGRMESQFALAMPRGSEA
AQLVRDAITAINTNPQLAECTPASVLGALMTCAQLGLRPGVLGHA
WVLPFRSKGVMQAQLVIGYQGLVELAHRTGQVASLIAREVHERDH
FDVDYGLADSLIHKPLLNGDRGPVTGYYAIVKFKGGGHSFIYASK
ADVEAHRDKFSKMKSFGPWVDNFDSMALKTVVRMLAKWMPKSTEF
ANAISADEGVRVDYSPTADVAQATEYVQPQLEEAPVEGVVVSEGG
ES
CCZ61365.1
[Clostridium hathewayi CAG: 224]
(SEQ ID NO: 328)
ANDIRGELARRASGTETQAVKLTKNMSIPDMIKALEPEIKRALPT
ILTPERFTRIALSAINNTPKLAECSPMSFIAALMNAAQLGLEPNT
PLGQAFLIPYKVKGSLECQFQIGYRGMIDLAYRNERVQSIEAHTV
YENDVFEYELGLNPRLVHIPTMEEPGDPIAFYGIFRLDNGGFRFE
VMNKNAIDAYAARYSKAYDSASSPWKNNYESMACKTVLKQLLKYS
PMKSEFQKAVSMDESVKTELSVDMSEVQNVNLIEETQEDAA
WP_068720576.1 RecT
[Veillonellaceae bacterium DNF00626]
(SEQ ID NO: 329)
KTTGGLQQQQQQQAQALQNGGTTLKGYLQAMMPEIKKALPTVMTP
ERFTRIVMTTISTNPALQNCTPQSFLGAVMQAAQLGVEPNTPLGQ
AYLIPYGNQVQFQLGYKGLIDLAYRSGEVQSLQAHEVYQNDTFEY
ELGLNPKLKHIPALTNRGDVILYYAVIKFKNGGEGFEVMSKEDVE
AFAKSKSKTYGRGPWQTDFDEMAKKTVLKKVLKYAPMKTDFIRAV
ATDETVKSSVAEQMADLPDETVTIDTEAQVVVDKETGEVKS
WP_037404193.1 RecT
[Solobacterium moorei]
(SEQ ID NO: 330)
TEIKAAKAPATVAKAGVSTQNKTIKDYITIMKPEIEKALPSTITP
ERFTRITLSAVSNNPKLQACSPSTFLSAMMQSAQLGLEPNTPLGQ
AYLIPYGNSCQFQLGYKGLLQLAYNSGQIKTIRTETVYENDEFKY
ELGLHSDLVHVPAMSNRGNPTAYYAVIEYTNGGYGFEVMSHDDVL
EHAKKFSKTFNNGPWQSDFESMAKKTVLKQALKYAPLSTELVSKI
NTDETVKSSISDHMEEVKNDIDLSQIIDAETGEIHE
WP_027347470.1 RecT
[Helcococcus sueciensis]
(SEQ ID NO: 331)
AQAKELLENKTNNTVKKSEKQTMENLLTLMADEIKKALPENVKSE
RFRRIALTAFNGNKDLQQCEPTSFLAAMMQSAQLGLEPNTPLGQA
YLIPYNNSKKNIKEVQFQVGYKGMLDLAHRTNQYKNIQANIVYEK
DEFDIEYGLNPKLKHIPNMKEDRGQAIGYYAVYNLINGGQGFEYM
TRAEVEKHAQKFSKTYRNGPWQTDFDEMAKKTVLKKVLKYAPMST
ELQEATAIDERVVNEENIKSKNEDKFVDVDWSYVDDVEEDVIE
WP_072526012.1 RecT
[Clostridium] sp. Marseille-P3244
(SEQ ID NO: 332)
AARATNSVKEELAKKAETKAVGEKKLTRSMSIADLIKAMAPEIKK
ALPEVITPERFTRMALSALNTTPKLQECTQMSFLAALMNAAQLGL
EPNTPLGQAYLIPFNNKGTMECQFQIGYKGLIDLGYRNPQMQIIS
AQAVYENDEFEYELGLNPKLEHRPALHDRGELRLFYGLFKLVNGG
FGFEVMSKEAVDAYAKEYSKSFDSSFSPWKTNYEAMAKKTVIKQA
LKYAPIKADFRKALSTDETIKNEIAEDMSEIHGEDIFDAEYTEQT
A
WP_092453396.1 RecT
[Clostridium fimetarium]
(SEQ ID NO: 333)
ETIDIKQELASQAQTDSKKEVKLTKAMSIAEMIKAMMPEIKRALP
SMITPERFTRIALSALNNTPELQACTPMSFISALLNAAQLGLEIN
SPLGHAYLIPYKNKGVLECQFQIGYLGLIALAYRNELMQTIQAQC
VYENDEFLYEYGLNPKLVHRPATSDRGEPVFFYGLFKMINSGFGF
CVMSKQEMDEFARTYSKGLASSFSPWKTSYNEMAKKTVIKQALKY
APIKTDFQKALSTDESIKYAISEDMTEAVNEIVSQNTEVA
WP_027295741.1 RecT
[Robinsoniella] sp. KNHs210
(SEQ ID NO: 334)
TTRTGNIKEELAKKAEGTNGDTRLTKAMSIADLIKAMEPEIKKAL
PEVITPERFTRMALSALNTTPKLRECTQISFLAAMMNAAQLGLEP
NTPLGQAYLIPFNNKGTMECQFQIGYKGMIDLSYRNPQMQMISAQ
AVYENDEFKYELGLNPTLIHRPVLRGRGEVILFYGLFKLINGGYG
FEVMSKEEMDAYAKAYSKAIDSSFSPWKSNYNGMAKKTVIKQVLK
YAPIKADFRKALSSDETIKNEISENMSEIHGEIIFDTDYMEESA
WP_117768035.1 RecT
[Blautia] sp. OF03-15BH
(SEQ ID NO: 335)
NVKEELAQKAEITQKEVKLKKSMSISDMIRALQPEIKKALPSVVT
PERFIRMALSALNTTPKLAECSQISVLAALMNAAQLGLEPNTPMG
QAYLIPFNNKGKMECQFQIGYKGLLELVYRNPAIQIIQAQTVYEN
DYFEYELGLNSRLIHRPELEDRGEIRLFYGLFKMVNGGYGFEVMS
RQEMDQYAARYSKSFASGFSPWENNYEDMAKKTMIKRVLKYAPVK
IETARALINDESIKLHLSEDMSEVENETVVDGQAEEKAA
SCJ42694.1
[Ruminococcus] sp.
(SEQ ID NO: 336)
GTSIQKNVENNALQKEKMPTMQAYIKKMEGEIKKALPSVMTPERF
TRITLSALSTNPKLAATTPGSFLGAMMTAAQLGLEPNTPLGQAYL
IPYSNKGKLECQFQIGYKGLIDLAYRSGSISVIQAHTVYENDDFE
YELGLDPKLKHIPSKSADKGNPAWFYAVFKTKDGGYGFEVMSIED
IRSHAAKYSQSYNSAYSPWKTNFEEMAKKTVLKKALKYAPLKSDF
VRQISTDETIKTKLSDDMFSVPAETIEVEGIEVDTETGEITEVDH
A
WP_092724975.1 RecT
[Romboutsia lituseburensis]
(SEQ ID NO: 337)
SNLKNVLKNQEDKGQGITVNPTYAMKQLMIKMKNDIDLALPKNLS
SERFQKVSMSAFNNNEKLQNCEPTTFIAAMMQSAQLGLEPNTPLG
QVYLIPHNLNGVDKVQFQVGYKGLLQLAHRSGKLKTLYAHEVKEN
DEFEIDYGLEQKLIHKPLLKGNRGDVIGYYAVYHLEPSGYSFEFM
TYDEVAKHGKKYSKDFEGGIWEKDFDSMAKKTVIKKLLKYAPLSI
EMQKAVAFDESVKSSIDSDMLLVESIGE
KKZ74881.1 VO63_05385
[Streptomyces showdoensis]
(SEQ ID NO: 338)
TSDARNAVARRAANVGQVEQAGEQPKPTMAQQIERMKPEIARALP
KHMDADRIARIALTLIRKNPDLANCTTESFLGALMTCSQLGFEPG
SPTQEAYIIPRKGQAEFQLGYQGMVTLFYQHPMASSVKVETVREN
DYFEHEEGLEERLIHRPFADGPRGKAIAYYSVARLINGGRTFKVM
YPAEIEERRQKLPSKNSPAWRDNYDEMAKKTVLRNHFKALPKSAE
LARALAHDGTVRTDWQPDAIDVPPEYLSEPQRPELEAGAQ
WP_055284109.1 RecT
[Dorea longicatena]
(SEQ ID NO: 339)
TVGKTDEIKQELARKVENTKAGTKLKKSMSIADMIKVMEPQIKKA
LPEVITPERFTRMALSALNTTPKLNECTPMSFLAALMNAAQLGLE
PNTPLGQAFLIPYNNKGKMECQFQLGYKGLIDLSYRNPNMQIITA
HTVYENDEFEYELGLNPCLDHRPTLGERGEIRLFYGLFKLTNGGF
GFEVMSKTAMDDFAKEYSKAFDSSFSPWRTNYESMALKTIIKKAL
KYAPLKSEFRNALSTDETIKNEIGADMSEINSENIFDTVYQEECA
SDL28883.1 RecT
[Streptomyces indicus]
(SEQ ID NO: 340)
STDARNAVARRAETVGQVEQQAQQQPTLAQQIERMKPEMERALPK
HMSADRMARIALTLIRKNPDLATCNTQSFLGALMTCSQLGFEPGS
PTQEAYIIPRKGNAEFQLGYQGMVTLFYQHPMASSIKVETVREND
YFEHEEGLEERLVHRPCATGPRGRAIAYYSVARLINGGRTFKVMY
PDEIEERRQKLPSKNSPAWRDNYDEMAKKTVLRNHFKALPKSAQL
ARALAHDGTVRTDATADVIDVAPEYPQRPELEAGPTA
WP_145458209.1 RecT
[Staphylococcus pettenkoferi]
(SEQ ID NO: 341)
ATQKDFKNQISQKETQQKQEVQKKKKGPRQQVSDLLDRMAPEIEK
ALPNHLSADRMARVAMTAVSSNPKLLECDPKSFIGAVMQSAQLGL
EPNTALGEAYLVPYAGKVNFQLSYLGLINLATRSGQYKAIYAHEV
YAEDEFRYQYGLHKDLIHKPVDNPKGKPIGYYAVYHLLNGGYDFV
YWTTERIQKHAKKYSFAVQKGYQSPWNDEFDAMAKKTVLKDLLKY
APKSIEMNNAVRSDDKQSELSDEGVVIDVTNYDEENGEEK
WP_117787252.1 RecT
[Tyzzerella nexilis]
(SEQ ID NO: 342)
AGVKEELAKKAESTKGETKLTKSMSIADLIKAMEPEIKKALPEVI
TPERFTRMALSALNTTPKLRECTQMSFLAALMNAAQLGLEPNTPL
GQAYLIPYNNKGVMECQFQIGYKGLIDLSYRNPQMQIISAQAVYE
NDDFSYELGLNPKLEHCPTLGERGEVRLFYGFFKLVNGGFGFEVM
SKTAMDEYAKEYSKAFDSSFSPWKSNYIGMAKKTVIKQALKYAPL
KTDFRKALSNDETIKTELSDDMSDIHGEEIWDVEYQEKTA
WP_073112630.1 RecT
[Hespellia stercorisuis]
(SEQ ID NO: 343)
ADIKEELAKKVAEGTEDKKKLTKSMSIADLIKAMEPEIKKALPEV
ITPERFTRMALSALNTTPKLKECTQTSFLTALMNAAQLGLEPNTP
LGQAYLIPYKNKGNLECQFQIGYKGLIDLSYRNRQMQIIQAQAVY
ENDEFEYELGLNPVLVHRPALQNRGAVKLFYGIFKLINGGFGFEV
MSKADMDAYAKEYSKAFDSSFSPWKSNYIGMAKKTVIKQAIKYAP
LKTDFRKALSTDETIKTEFCEDMSEVQCKDIWDTEYKERSA
CDD36322.1
[Roseburia sp. CAG: 309]
(SEQ ID NO: 344)
DVKNELAKKAENTGKVKLTKSMSIADMIKTLEPEIARALPSVITP
ERFTRMALNALNNTPKLAECTQMSFLAALMNAAQLGLEPNTPLGQ
AYLIPYKNKGVLECQFQIGYKGMLDLVYRNEMVQTVQAQVVYQND
EFHYALGLTGRLEHIPTLRDRGEPYAFYALFKLENGGYGFEVMSK
TDMDAFALQYSKGISSEYSPWKTNYIDMAKKTVIKKVLKYAPLKT
EFQRALSNDETIKTHFAVDMSEVEPETVIDMEEGELLESAS
WP_128520904.1 RecT
[Absicoccus porci]
(SEQ ID NO: 345)
TTTNQQGMITKKANNSVAKKTNRTMKDYITMYQGEIAKALPSVMT
PERFVRIATTAVTNTPKLASCTPQSFIGALLNAAQLGLEPNTPLG
QAYLIPYGNQCQFQIGYLGMVELAQRAGTNVDAHVVYANDEFDYS
LGLHPDIKHVPAMKDRGEAIAYYAVWHNGENFGFEVMSREDVEKH
MKKYSKTYSNGPWKTEFDEMAKKTVLKRALKYAPKKTDLARAVMQ
DETIKQFNPKADNDMADAKNDFFDVEYDEVDENTDPVTGEVK
GAK01483.1 RecT
[Geomicrobium sp. JCM 19055]
(SEQ ID NO: 346)
GYKGMIDLARRSGHIKSIYAHTVHANDEFEYELGLEPKLVHKPAT
GDRGNMEYAYAVAHFVDGGYQFEVFSHHDIEQVKKRSKAGNFGPW
KTDYEEMAKKTVVRRMFKYLPISIEIQQHASQDETVRRDITEEAE
KVDNIIDLPNYEDPNNIDVPDEEQDEQKDEKQKQQGSAEEIALDF
K
WP_135329961.1 RecT
[Streptomyces] sp. MZ04
(SEQ ID NO: 347)
STNLAARVEARRQNPTTKQPARRGKAAQQPTLVQFVQSMRGEIAR
ALPSHVASPERIARIALTELRRVDHLAECTQESFGGALMTCAALG
LEPGGVGGEAYLLPFWNKKVRAYEVTLVIGYQGMVRLFWQHPAAA
GLAAHTVHEGDEFDFEYGLEPFLRHKPARTGRGKPTDYYAVAKMA
NGGSAFVVMNVEDIEAIRHRSKARDAGPWSTDYGLRRHGAQDLHS
AVVQVAAEVC
WP_079588582.1 RecT
[Acetoanaerobium noterae]
(SEQ ID NO: 348)
SNLKNELAKKANNSVTDGNKEPQTIKDWIKVMEPAIKKALPSVIT
PERFTRMALTAISVNPKLAECTPKSFMGSLMNAAQLGLEPNTPLG
QAYLIPYKNKGNMEVQFQIGYKGLIELAYRSGEFANIYAKEVFEN
DEFEYEFGLEPVLKHKPASGNRGEVIAYYAVFKLTNGGFGFEVMS
KEDITNHAKTYSQAYSSSYSPWSKNFDEMAKKTVLKKVLKYAPIK
VEFVKQIVQDSTIKTEINSDMTEVESQNVFEAEETDYEVIDQEETK
WP_107635892.1 RecT
[Staphylococcus haemolyticus]
(SEQ ID NO: 349)
ATQNEFKNQLAKKEDKGNTNAPTQTKSTNPRTIAQNYLAKMKPEI
EKALPAHMSHERMTRIALSAVNSNPELTEVILNNPTSFLGALMQS
AQLGLEPNTNLGHAYLIPYYDKNSGKKIVNLQLGYMGLLDLAHRS
GMYQKIFAMPVYKDDYFEYQYGTNEKLNHVPAQVSKGEPIGYYAF
YKLINGGVHFVYWSRQKMQMHKDRYTRRGSVWNNNFDAMALKTVI
KDVLKYAPKSVEMGEAVQSDENNFEFNEDSKVIDVTDYETEENK
WP_107638953.1 RecT
[Staphylococcus hominis]
(SEQ ID NO: 350)
ATANDFKNQVTKKESDNTKESSNKKTELATTSPRQVAQNYLEKMK
PEIAKALPAHMSHERMTRIALSAVNSNPQLTEVILNNPTSFLGAL
MQSAQLGLEPNTSLGHAYLIPYNFKGKKIVNLQLGYMGLLELAHR
SGLYKKIFAMPVFKDDFFEYQYGTNEKLNHIPAQVQNGDAVGYYA
FYQLTNGGVHFVYWSRQKMERHKDLYTRKGSVWNTNFDAMALKTV
IKDVLKYAPKSVEMSSAVQSDNSNFEFSEDSSTVIDVTDYETEDN
K
SUY49750.1 RecT
[Lacrimispora sphenoides]
(SEQ ID NO: 351)
ADVKQELEKRAAGSGGQSVKLTKNMTIVDMVKALEPEIKRALPCI
LTPERFSRMALSAINNTPKLGECTPMSFIAALMNAAQLGLEPNTP
LGQAYLIPYKNKGVLECQFQLGYRGLIDLAYRNERMQSIEAQVVY
DNDEFSYELGLHPSLIHRPTFDEPGEIQAFYAIFRLDNGGFRFEV
MSKNYVDSLCHALFKSIYFRFQSLEK
CDE68291.1
[Clostridium] sp. CAG: 277
(SEQ ID NO: 352)
DFKEELAAKAEVAATTKKSDGVKLTKNMSIVDMIKALEPEIKRAL
PSVLTPERFVRMALTAVNNTPALAQCTPMSFIAALMNAAQLGLEP
NTPLGQAYLIPYKNKKKGVVECQFQIGYKGMIDLVYRNDNVQTIQ
AHIVRENDHFEYELGLESKLRHIPAMEGRGEMMYVYALFKLTNGG
YGFEVLNKEAVIAHAERYSPSYDGFSPWKTDFESKGLELFLILDL
SSKQSGK
WP_060905391.1 RecT
[Streptomyces scabiei]
(SEQ ID NO: 353)
DADRMARIALTLIRKNPDLATCSGESFLGALMTCSQLGFEPGSPT
QEAFIVPYKGEATFQLGYQGMVTLFYQHPMASSVKVETVRENDYF
EHEEGLEEKLVHRPCKTGPRGKAIAYYSVARLINGGRTFKVMYPA
EIEERREKLPSKNSPAWRNSYDEMAKKTVLRNHFKALPKSAELAR
AMAHDGTVRTDWQPDAIDVPPEYLSEPQRPELGTGSTQ
WP_146678271.1 RecT
[Pirellula] sp. SH-Sr6A
(SEQ ID NO: 354)
SEATKEAKPETAIAKKPNGIKDWLKSDALKTQIASVAPKHMAPER
VMRIALNAVSRTPKLADCTPESFMRCLLDLSSWGLEPDGRHAHLI
PYGTECTLVLDYKGLVTLAYRSGWVKKIHADVVFEGDIFVYSLGT
VCQHIPWEFRDDANKPEHKGHFRAAYCVVTMADGIEKHEVMTASE
IDAIKAKSRSGNSGPWKDHYTEMAKKTVYRRASKWLPLSPEQADA
MERDDDRIIDAVSVAVTQRLSKAAMPLIGANETGDTE
WP_126032909.1 RecT
[Bifidobacterium castoris]
(SEQ ID NO: 355)
GALATTAKNNELTTMNTMGDIHALIRGRRAQIESVMSGVLTPERL
YSLLQSAVSHEPKLLQCTPESIVACCMKCAVLGLEPSNVDGLGKA
YILPYGNKNYQTGQVEATFILGYKGMIELARRSGEIKSLNVTPVF
EDDGIKLFMDEAGQPYIKAGEVNPLANHTPDKLMFVFLNAEFTNG
GHYRTYMTRAEIDAAKKRSSAGDRGPWKTDYVAMARKTVVRRAFP
YLPVSTEAQSAAVEDETTPHFDFLDRNTTPVGEPSDVMQEATA
WP_114599505.1 RecT
[Staphylococcus warneri]
(SEQ ID NO: 356)
ATQNDFKNQITDKKENKPQQSTNPRQVASDLLERMKPEIAKALPA
HMSQDRMTRIALSAVNSNPKLSEVILNNPTSFLGALMQSAQLGLE
PNTNLGHAYLIPYGNIVQLQLGYLGLLELAYRSGKYQKIMAMPVY
KDDFFEYQYGTDEKLNHIPAQQQTGDAVGYYAFYKLINGGTHFVY
WSRQKMNMHQQQYSKGGNVWRNNFDAMALKTVIKDVLKYAPKSIE
MGEAVTSDNNNFDFKDGGDIIDVTDYETEEN
SCQ72869.1 RecT protein
[Propionibacterium freudenreichii]
(SEQ ID NO: 357)
TQQMPIKAQGEPTKELQQKAAVDRFNATLHQMQNEIARALPKHMT
GDRFVRIVLTEVRKNPTLALCDPLTMFGSLLTAAALGLEPGLNGE
CWLVPRKNHGTLEAQLQVGYRGVVKLFWQNPAATYLDTGYVCERD
EFRFAKGLNPILEHTPAEGDRGKVVRYYAVAGLNTGARVFDVFTP
AQIKTLRGGKVGSNGDIPDPEHWMERKTALLQVLKLMPKSTQLAA
VPAADGRAHTISDAQQIFGGVDPTTGEVLDAEPVEDGAA
WP_127100780.1 RecT
[Asaia] sp. W19
(SEQ ID NO: 358)
SNALATPTEKLRTQITSMTGEFRNALPSHIKPEKFQRVVMTVVQQ
NQGLMNADRKSLLASCLKCAADGLIPDGREAALVMFGQQVQYMPM
LAGIQKRIRNSGEIASIQAHVIYENDHFIWHQGIDASIEHRPLFP
GDRGKAIGAYAVAKFKDGSDPQFEVMDVAAIEKVRAVSRAGKSGP
WVQWWDEMARKTVFRRLSKWLPMDTEAEDLMRRDDENDAQDVAAP
TIRVEAEAPSKLDALEHDDDGVVLEETRELEGSAA
EIC09117.1 RecT protein
[Microbacterium laevaniformans OR221]
(SEQ ID NO: 359)
TDLSTVAAAAKQNPTMKDLVEAQLPAIERQLGGTMNSDAFVRAVL
SEITKSPDLMQADPKTLLGGVMLAAQLRLEIGSGLGEFYLTPRKD
HGRMICLPIVGFQGMVKLALRSEFVTNVQAFIVREGDDFTYGANA
ERGMFYDWTPKDFEEKRPMVGVVATARMKQGGTTWAYLTREQVED
RRPSYWQKTPWGSHPDEMAKKTAVRALAKYLPKATDLGRAIEADE
QKVQHVKGLDEVTVTRLDDEPETVVVQETTDAWAATPVAEVQP
WP_136046271.1 RecT
[Microbacterium] sp. K41
(SEQ ID NO: 360)
SKDLSTAAAAAKSQPTMKDLVEAQLPAIERQLGGAMNSDAFVRAV
LSEIGKSPDLMNADPKTLLGGVMLAAQLRLEIGSGLGEFYLTPRK
DRGRQICLPIIGYQGMIKLALRSEYVLNVQAFLVREGDDFTYGGN
SERGMFYDWTPKDFEESRPWIGVVATAKMRGGGTTWVYLTRTQVI
DRRPSYWASTPWKTNEDEMVKKTAVRALAKFLPKSTDLGRALEAD
EAKVQHLKGVDEVQVTRLDDDAETFVVQEQDPMSRTPEEQAEDEA
NR
WP_136309287.1 RecT
[Streptococcus pyogenes]
(SEQ ID NO: 361)
SDLSVAAAAAKTQPTMKDLVEAQLPAIERQLGGAMNSAAFVRAVL
SEIGKSPDLMAADPKTLLGGVMLAAQLRLEIGSGLGEFYLTPRKE
RGRQICLPIIGYQGLIKLALRSEFVMNVQAFLVRQGDQFSYGANA
ERGMFYDWVPQDFEETRDWIGVVATARMRSGGTTWVYLTRTQVID
RRPSYWNSTPWKTNEDEMVKKTAVRALAKFLPKSTDLGRALEADE
AKVQTLRGLDEVEVTRLDDEADTVVVQEQNPMSRTPEEQAEDAEA
QR
WP_110990907.1 RecT
[Mesotoga] sp. TolDC
(SEQ ID NO: 362)
KASEIASMVKKEDERRNHKPDPLAGIVKNLTSIKGEIANALPDAG
ITPERMIRIVVTLLRQNKSLAEAAMQNPASLLGAVMMAAQLGLDP
TNGLDQCALVPRKGKVCFDIMYEGLVELGYRSDRMESIVARTVYE
KDTFSLKYGLNEELVHIPYLDGDPGESKGYYMVGKLKGGGNIIVY
MTKEQVHKIRDRYSVAYKAGLSGSRKDSPWFTSEDRMGEKTVVKA
GFRWIPKSPIIRTALALDETAREASRLPMRN
WP_109196224.1 RecT
[Streptomyces] sp. CS014
(SEQ ID NO: 363)
TENTVTAAVAVRDTGPAAQIEAYRDEYAALVPSHINADQWVRLAV
GAIRGNEDLTNAARTDIGVFLRELKTAARLGLEPGTEQFYLTPRK
SKAHRGQKIIKGIVGYQGIIELIYRAGAVSSVIVESVRANDTFRY
VIGRDERPVHEIDWFGGDRGDLVGVYAYATMKDGATSKVVVLNHA
QVMQIRAKSDSKHSEYSPWNTNPESMWLKSAVRQLMKWVPTSAEY
MREQLRAQAEVAAEQPPAADLPPMPSVELNDEDEAVDAELVDEEA
WP_068202759.1 RecT
[Isoptericola dokdonensis]
(SEQ ID NO: 364)
TQDLATAIADQQPAQRRTAFDLVESMRGELHKALPEHASIDNFLR
LALTELKMNPQLGNCSGESLLGALMTAARVGLEVGGPLGQFYLTP
RRLKRDGWAVVPIVGYRGLITLARRAGVGQVNAVVVHEGDTFREG
ASSERGFFFDWEPAVERGKPVGALAAARLAGGDVQHRYLSLAEVH
ERRDRGGFKDGSNSPWATDYDAMVRKTALRALVPLLPQSTALSFA
VQADEQVQRYDAGDIDIPALDETDTEDTK
WP_114797327.1 RecT
[Gaiella occulta]
(SEQ ID NO: 365)
STAVARRDPVAEVCTTIASKEFEAKIVQALPDGVTPARFVRTTLT
AIQQNPDVVKGTRQSLYNAVIRCAQDGLLPDGREAALVVFRAKGT
DVVQYLPMIGGLRKIAAEYGIKIETAVVYERDKFEWELGFEPRVL
HVPPALGEDRGEPIGAYAVATDKLGRKYVEVMSRQEIEEVRKVSR
AATSEYGPWVKWWAEMARKTVGRRLFKQLPLHDLDERGERVISAS
DAEISFSPSGLDSLPHVDPSEPEEVLTGDVMDDDDDDGIPFGEPA
A
PAV10712.1 CBG25_01455
[Arsenophonus sp. ENCA]
(SEQ ID NO: 366)
NTELETMNNVYDNLQSVIMQQGIAALLPAQVTPEQFTRTAATALI
ENVDLQNADKQSLVLALTRCAKDGLMPDGREAALVVRSTKVNKQF
VKKAVYMPMVDGVIKRARQSGQVANIIAKVVYSQDEFEYVIDENG
EHLTHRPAFVDGDDIVKVYAFAKLNSGELVVEVMSRAGVEKIRDT
VQSAKYDSSPWVKWFDRMALKTVIHRLARRLPCASELFSLFEVYE
DANSTEKTLRMAPASFKRLSIN
WP_147981944.1 RecT
[Streptomyces] sp. ms191
(SEQ ID NO: 367)
VEHYKADLAQVMPSHVKPDTFIRLAVGVLRRDRNLAQAAQNNPAA
LMGALMDAAQLGLTPGTEQFYLVPRKKAGRLEVQGIRGYQGEIEL
IYRAGAVSSVIVEVVRQADTFRYSPGRDERPEHEIDWDAEDRGPL
RLVYAYAVMKDGATSKVVVLNRAQVMKAKAMSQGSDSAYSPWQKH
EEAMWMKTAAHRLTKWVPTSAEYMREQLRAAAEVAAEHRPTPVAA
APGMPSVPGEDEAIEAEFVDEDEVA
BAQ93806.1 phage RecT family (TIGR00616)
[uncultured Mediterranean phage uvMED]
(SEQ ID NO: 368)
TSSITPLVAMQGTLEKMADKFTEALPRQMDVNKFISVAKLTLNKN
PRLLQADKTSLMQTFMKAAQDGLYLDGKEAAAVQYGQSVQYIPMV
EGIIKVLHNSGLIKTISAEVVYENDFFDYELGTAPKITHKPLIVG
DRGKPMCVYAVAITTNDGEYYEVMNMDQINQCRQVSKASSSPHSP
WVKWFDQMAKKTVIHRIAKRLPKNDAINSVVTVDDEPNFQQAVNV
TPSEPKDSLSRLRDSIGMEGKDVEQAANDLLEKYNKEAREE
WP_061405262.1 RecT
[Streptomyces]
(multispecies)
(SEQ ID NO: 369)
SQISNALATRDQGPAAQIEQYRDEYAALVPSHVNADQWVRLAVGA
VRGDEKLMEAAQNDIGLFLREMKTAARLGLEPGTEQFYLTPRKSK
PHGGRKVIKGIVGYQGIVELIYRAGAASTVIVEAVRENDTFRYVP
GRDDRPVHEIDWFANDRGPLVGVYAYAVMKDGAVSKVVVLNRSRV
MEFKAKSDSKHSEYSPWNTNEEAMWLKSAVRQLAKWVPTSAEYRR
DQLLAHTETADSVVASVSTAPLPPQPSALDDADPDDDGPIDAELV
D
WP_114014965.1 RecT
[Streptomyces reniochalinae]
(SEQ ID NO: 370)
SQISNAVAKRDNSPGAMVQQYKADFSTVLPDHVKPDTWVRLAQGV
LRRDKNLAQAAERNPGSLMTALLDCARLGHEPGTESFYLVPFGGE
VQGIEGYRGVVERMYRAGAIASVKAEVVCQGDDFDYQPDMDKPRH
RVDWFGDRGPIVGAYAYAIFKDGSTSRVAVINRAYIDKVKKESKG
SDRATSPWMKWEEQMVLKTVAKRLEPWVPTSNEWRREQLRAAREV
ANEPTPPTTPAPPAPEQVDPDTGEVIDGELVDDTPTQ
WP_027699748.1 RecT
[Weissella oryzae]
(SEQ ID NO: 371)
SNNLTSAQYFNAPNIKGKFEEVLGKNANGYVTSLLSVINGSQQLQ
RAEPSSIMVAAMKAATLNLPIESSLGFAYIVPYGNNAQFQIGYKG
LIQLALRSGQIKGLNSGVVYETQFISYDPLFEELEIDFKKPAEGK
IAGYFASMKLINGFSKVVYWTKEQVEQHRDRFSKGKNNGPWKSDE
DAMAQKTVMKAMISKYAPLNQEMQQAIVEDSESELTVPRDVTTSN
EAAELNSLLTTPKVQEGANTDLSEPFPNAEETQLFDDLASVTGD
SYW13692.1 Phage RecT family protein
[Oenococcus oeni]
(SEQ ID NO: 372)
SNELKTILNAPTTKEKFDEVLGRNAQGYINSVLNAVGNSKLLQNA
SPNSILSGAMKAATLNLSIDPNLGYAYFVPYGHEAQLQIGYQGLI
QLAQRSGQIKILNAAPIYDEQFKSLDPVTGKLTLNKKIVPDTNKK
PTGYVAYLKTVTGFEHTEFMSYADIEKFAKRFSKSFNSSTSPWKT
DFNAMAKKTLIKQVLKYAPMSIDLQTAVSADNDDIEPKDITPDED
KETVDKISNLISDNKQDDTLSQLEEVANANN
WP_141158250.1 RecT
[Pseudarthrobacter] sp. NIBRBAC000502771
(SEQ ID NO: 373)
TSQLAEATAAKAVEQRKNPTARDLIQAQQAAIETQLAGAMNSAAF
VRAAISSVSASPQLQQATPASLLGGIMLAAQLKLEIGPALGHFHL
TPRMVSKKDGDNWVEVWTCLPIIGYQGYIELAYRSGRIEKIESLL
VRKGDKFDHGANSERGRFFDWAPADYEETREWTGVIALAKIKGAG
TVWAYLPKEKVIARRPDRWEKTPWATNEEEMARKSGIRALAPYLP
KSTELGKALEADEHKVEHIAGVHDLVVSKAEDEPLEEPTA
TAK04183.1 EPO34_03495
[Patescibacteria group bacterium]
(SEQ ID NO: 374)
TNQPTTHVATTPNQRPATTLEQFRHQLVGDYQKQVLNYFNGQKEK
AMKFMSAVVYSAQKNPALLECDRTTLLHAFMACAEYQLYPSSVSG
EAYVIPYKGKAQFQLGYQGIITLLYRAGVEAVNAQIICENDAFEY
EEGLEPNLVHKPNVLKDRGKPIGVYAIAAINGHKLFKVLSEAEVM
KFKGFSQSKNSEYTPWNPDNDPELWMWRKTAIKQLSKLLPKNDAL
QKAISEDNQDSVIEARRSTLDAGGPAVGRALHDPNASNEPEGK
WP_092601202.1 RecT
[Actinopolyspora xinjiangensis]
(SEQ ID NO: 375)
TGQTIGTAVAKKDDENPSAIIATNRADLARVMPSHVRTDSWVRIA
QGIVRRDKNLAHAARQSPGTLMVALMEAARLGLEPGTEQYYLTPR
KNKGKPEVLGIPGYQGLIELMYRSGAVSSVVVETVRENDTFQWAP
GRMERPEHEADWFAINGERGQLRGVYAYAIMSNGATSKVVVLNRN
DIARARDSAQGADSEHSPWKNHEEAMWLKTAARRLAKWVPTSTED
RRIVQGVAERSDQPTEAPLDLTDEPDTDQPIEGELVDEEATQ
WP_067024969.1 RecT
[Mycobacterium] sp. 1245499.0
(SEQ ID NO: 376)
TTQPEYKPVAQGADKQMTTGKLLKMLEPEIGRALPKGMDPDRICR
LVMTEVRKNPMLTQCTQESFAGALLTASALGLEPGVNGEAWLVPY
RDRKRGIVECQFIMGYNGVAKLFWQSPHADRLDAQLVCANDHFRY
VKGLSPILEHVESDGDRGDPIAYYAIVGVKGAQPMWDVFKPEAIA
QLRGGRVGTKGDIDDPQRWMERKTALKQVLKLAPKSTRLDLAIRA
DERSGSDLYKSQGMEVHAIEPGFIETEAEPETQEQ
WP_075737485.1 RecT
[Streptomyces acidiscabies]
(SEQ ID NO: 377)
TDNAISNAIATRDNGPEAIVQQHRDDLTLVLPAHHKGETWMRLAT
GALRRDANLRQTAARNPGSLMNALLECARLGHEPGTESFYLVPFG
NEVQGIEGYRGIVERIYRAGAVKAVKAEVVYENDHFRYHPGMDRP
EHEPDYFADRGRIIGAYAYGVFQDGSTSRVVVINRAYIDKVKKES
KGSDRASSPWVKWEEGMVLKTVARRLEPWVPTAVEWRTEPTPASA
AEATAPVGDGVKAIAAPAPTSPYDDEGPIEGEFVDEYDGGAA
AKT73182.1 RecT (prophage associated)
[Yersinia pestis]
(SEQ ID NO: 378)
NQVATLESIHADLSSALTRQGIQSLLPSHVSPEQFTRTAATALVA
DPELQNADRQSLVMSLIRCAQDGLVPDGREAAMVVYNTKQGDQWV
KKAQYLPMVDGVLKRARQSGQVANITGKVVHMADKFDYWVDENGE
HIEHRPAFENHGEIRLVYAFAKLTSGEIVVEVMSRSEVEKVRDAT
AKKDRDGKPKVPAVWQKWFDRMALKTVLHRLARRLPCASELYSLL
DVNQIADEAEKPAECGAQRESSTTAA
WP_123127078.1 RecT
[Rufibacter latericius]
(SEQ ID NO: 379)
SNQLQVAREQVISAQKSFKNVPNNKLDFEREAGFAMQMIQSNPFL
ASMDANSIRNCIVNVALTGLTLNPVLKLAYLVPRKGKLILDPSYM
GLINVLVTSGAAKKIEADVVCENDFFDYEKGTNGFIKHKPSLSSR
GEIIAAYAIAHLPNGEVQFEIMNREELEKVRKSSEAAKKGSSPYD
GWASEMMRKAPIRRLFKYLPKHNIPDQVINTLSLDEQNNGVDFSA
QKQEAFKGKAADFFEDEPANTVDADYTDMSHEEADNELAA
WP_093587584.1 RecT
[unclassified Streptomyces]
(multispecies)
(SEQ ID NO: 380)
SQIGNAIATRDEGPAAQIEVYRDEYAALVPSHVNADQWVRLAVGA
IRGNDDLLKAAGNDIGLFLREMKTAARLGLEPGTEQFYLTPRKSK
AHGGRPVIKGIVGYQGIVELIYRAGAASTVIVEAVRQNDVFRYVP
GRDDRPVHEIDWFGQDRGPLVGVYAYAVMKDGAVSKVVVLNKARV
MELKAKSDSKNSPYSPWNTNEEAMWLKSGVRQLAKWVPTSAEFRR
DQLLAHTDTADGVIASVSAPPLPPQSAALEDLDPDDEGPIDGELL
DD
WP_030975214.1 RecT
[Streptomyces] sp. NRRL S-1824
(SEQ ID NO: 381)
SEISNAIATRDQGPAAQIEAYRDEYAALVPSHINVDQWVRLAAGA
IRGNEDLMEAARNDIGVFLRELKTAARLGLEPGTEQFYLTARKSK
AHGYALIIKGIVGYQGIVELIYRAGAVSSVIVEAVRANDTFSYVP
GRDDRPIHEIDWFGGDRGPLVGVYAYAVMKDGAVSKVVVMNHKRV
MEIKARSDSKNSQYSPWNTDEESMWLKSAIRQLAKWVPTSAEYKS
EQLRAHAEAIGELASVASAPLPPQPSVLDDVDPDDEGPIEGELVD
RKT60104.1 RecT
[Agromyces] sp. OV415
(SEQ ID NO: 382)
STTVALPAQKAEAVIQQVTGAANGFAAALADRIGPDRFVRAAVTS
IRTSPQLAQCEPLSILGGLFVAAQLALEVGGPRGLAYLVPYGREA
QLIVGYRGYVELFYRAGARKVEWFIVRDGDTFRQWSTGRGGRDYE
WTPLDDDSNRRPIGAVAQIQGAHGEFQFEHMTVDQINERRPKRAT
SGPWVDWYEEMALKTVMRQLAKTARQSTDDLAFAAANDGAVITQV
EGGQARVVHPATSEPEQPLSLDALERTPGELAEETNP
WP_017415747.1 RecT
[Clostridium tunisiense]
(SEQ ID NO: 383)
TTKANVTSVKNALKEQIQVQQVAAQTDTSFQGVLTKQLQHQFKAI
QSLVPKHVTPERLCRIGINAASRNPQLMNCTPETIVGAIVNCATL
GLEPNLLGHAYIVPFYNNKTGKMEAQFQVGYKGALDLIRRTGAVS
TLSAHEVYGPRSIFWTQYFY
RYE05836.1 EOP33_01060
[Rickettsiaceae bacterium]
(SEQ ID NO: 384)
TNSIETNIEDLSPGNQTKTQISETNAPIVSEIRTIKRDGVYDLCS
SRREKVLPFLGNNSQKFERLARSFAFEINTNPKLASCDQLSILQA
FYKCCEYGLDPASSLQQIWMIPYNGKIDFQIGYKGWLQLLWGSKL
ITNAYSCAVYQGDQFEYELGLNPNIKHIPQHKSINDVNELIATYG
VIKLKSNEVQLRVCWRDELEKSKKSSKSNGREDSPWNRHFEAMAL
IVPMRKMAKNLALALRAEDFDDEDYVNENNNQGMA
WP_052399147.1 RecT
[Francisella] sp. FSC1006
(SEQ ID NO: 385)
SNLVVAKQCLASAEKSFIGISGDEKKYKRECNFAVQSLQANSYML
QQANANPNSLRNAIINLASMGLTLNPAEKKAYLVPYGGKNPRVDL
QISYMGLIDLAISDGAIMWAQAKVVRQNDLFNITGVDTPPEHKYN
PFDSEQTRGDVIGVYCVAKFPNSDYITEIMSITDINSIKSRSSGV
KSGNTTPWDTDFDEMAKKTVIKRASKYWKGSSKLSKAIDFLNNEN
NEGINFNKQEEKPKQNINDLMNDDVVDIDSEVGDE
WP_067349107.1 RecT
[Streptomyces noursei]
(SEQ ID NO: 386)
TSPIRAAVARRAGDPAALISQYTADFAAVLPSHIKPATFVRLAQG
ILRRDEKLAQAAANDPGQFMSVLLDAARLGLEPGTEAYYLVPFKG
RVQGIVGWQGEIELMYRAGAISSVIVETVREDDVFVWTPGLVDRE
TPPRWEGPMSYPFHEVEWAGDRGPLRLVYAYAVMKGGATSKVVVL
NAQDIERAKKTSQGADSPSSPWRQHEAAMWSKTAVHRLAKYVPTS
AEYITAQVRAVRQADALSAPPVEEVVDVELVGDGQEQEARR
WP_143887802.1 RecT
[Streptococcus lutetiensis]
(SEQ ID NO: 387)
ANQLQMSHKDFFNRPAVKNKFSEVVSGKSDQFITSLLSVVNNNKL
LSKADNNSILNAAMKAATLNLPIEPSLGSAYIVPFKGQAQFQLGY
KGLIELAQRSGQYKSINAGVVYKAQFKSYDPLFETLDLDFNQPQD
EVIGYFACFELLNGFRKITYWTREEVYNHGKRFSKSFNNGPWKTD
FDAMAKKTLLKSIIGTYGPKSVDMQEAITDDNKTEYEKAEPIDVT
PQEENLTDLIGETPQEELPIANPETGEIQEEQTALFNQLGDLTDD
WP_073793143.1 RecT
[Streptomyces uncialis]
(SEQ ID NO: 388)
SQISTAIATRDNGPAAVVEQYRESLALVMPSHLQQRVGAWIRNTQ
GLLRRDSKLMEAAQNDVGQFVAVLMDAARLGLEPGTEHYYLVPRW
NNKKRATEVTGVRGYQGEIELMYRAGAVSSVIVEVVHTQDQFRFR
PGRDARPVHDIDWDLEDRGSLRLVYAYAVMKDGATSKVVVLNRQH
IAAARAKSDSAAKDWSPWNTDEEAMWLKTAAHRLTKWVPTSAEYL
REQIRAQVAVESEQRPEPLPVAPPPAPGTVDADPDDEGPIDGELV
D
WP_116200709.1 RecT
[Amycolatopsis circi]
(SEQ ID NO: 389)
ISQTVTTAVAQQKDSSPAALVRKYRTDFATVLPSHIKPETWLRIA
TGALRRSPQLANAAKRNPSSLLVALLEAARKGLEPGTEQFYLVPR
KGKNGPEVLGITGYQGEVELMYRAGAVSSVKVEVVREHDTFAYNP
GEHDRPVHEIDWRADRGDLVLTYAYAVMRDGATSNVVVLSADDIA
VILKKADGADSPFSPWQWNPKAMWLKSAARQLAKWVPTSAEYVRL
PDVPLESLPPAKPLDLPRVDDVVDAEIVEDWPTAPDDTADGAR
WP_020135111.1 RecT
[Streptomyces] sp. 351MFTsu5.1
(SEQ ID NO: 390)
SQISNAIEKRDQGPGAVIEQYKQELALVAASHVKVDTFARLAVGA
LRQNPKLAAAAQSNPGSLMSALMTAARLGLEPGTEQFYLRPIKRK
GVAEVQGIVGYQGIVELIYNAGAASSVVVEVVRANDQFNYVPGLH
ERPVHNVDWFGDRGDLVGVYAYAVMAGGATSKVVVLSRTHINRAK
AKSDGADSDYSPWRTDEEAMWLKTAARRLGKWVPTSAERLTMPAE
RTDTVLPVGSAAPALDAADPDEDEGPVDGELEPAGGWPETAQPPQ
WP_099421180.1 RecT
[Streptococcus macedonicus]
(SEQ ID NO: 391)
ANQMQVSHKDFFNSPAVKNKLSEVVGGKSDRFIASLLSILNNNKL
LSSADNNSILTAAMKAATLNLPIEPSLGFAYIVPYKRQAQFQLGY
KGLIQLAIRSGQIKSINSGVIYKAQFKSYDPLFETLEVDFSQPED
EVAGYFATIELLNGFKKLIYWTKERAYNHGKRFSKSFGNSPWQTD
EDAMAQKTLLKQIISKYAPLSVELQEAITADNENEDEKAAPIDVT
PQEESLSDLIGEAAQEELPAADPETGEIQEEQTALFEQLGDLTDD
WP_141925904.1 RecT
[Haloactinospora alba]
(SEQ ID NO: 392)
GQSVTNAVAQRDTSPSGMVGKYRDDFAQVMPSHVNGAGWVRIAQG
ILRRDAKLAEAARNAPQSLMSALMDAAQQGLTPGTTEFYLVPRKR
KGSLEVQGITGYQGEIELIYRAGAVASVVAEIVHEHDTFEWIPGK
HERPIHEADWFGNRGTMVGAYAYAVMNSGSTSKVVILNQHDIEKA
RAMSDGADSSYSPWQKWPESMWLKTAAHRLAKWVPTSAEYRHEQE
RARARSEDTEIPASPDSDVVHAEIVEENDDEQAT
WP_136710836.1 RecT
[Clostridium tyrobutyricum]
(SEQ ID NO: 393)
SDKKMVVLGESHKALSKLLETKQEALPKDFNKARFLQNCMTVLQD
TKDIDKCQPISVARTMLKGAFLGLDFFNRECYAIPYGGNLQFQTD
YKGEIKLAKKYSFNSIKDIYAKIVREGDDFQESIEDGRQTINFKP
LPFNNGEIIGAFAVCLFQDGSMLYETMTKQEIEDIRNNFSKAKNS
PAWVKTPGEMYKKTVLRRLCKLIELDFDSVETKKTYDETSEFEFG
SANHEVSNFDKDDSNIIEADAEIQDDVQEGDGEDE
WP_132110073.1 RecT
[Actinocrispum wychmicini]
(SEQ ID NO: 394)
SQTVTAAVAQRDNGAQALIAKYRTDFAQVLPSHLRPTTFVRLSQG
LLRRNVKLAEAAERNPASFLAALLECARLGHDPGTDQFALVPFND
RKRNTVEVVGIEQYQGVIERMYRAGAVRSVKAEVVRAADPFEYAP
DVMDRPGHKPNWFADRGELIGVYAYAEFFDGSTSRVVMMNRETVM
AHKAKSRGATSEDSPWQAWEESMWLKTAVHELEKWVPTSSEDRRA
ARDGTADPAPVEVPRVADEVLDADLVEDDHADHPTATPTGDVR
WP_125769509.1 RecT
[Companilactobacillus furfuricola]
(SEQ ID NO: 395)
VNNLAKLPIQTLVKEPKIVEKFESVLGNKSAQFVTSLINVVNSNQ
SLKNVDQMSVVASAMVAASLDLPINQDLGYMWLVPYGGKAQPQMG
YKGYIQLAQRTGQYKHLNAVAVYEDEFQSYNPLTEQLDYEPHFKD
RDSSEKPVGYVGYFELTSGFEKTVYWTRKQIDDHRQSFSKMSGKS
KPSGVWATNFDAMALKTVLRNLISKWGPMSVEMQKAYESDEHATT
ISANDIKDIEVQEQEPATDVSQLINGSATEVNVNDSTTNSKDSE
WP_004234437.1 RecT
[Streptococcus parauberis]
(SEQ ID NO: 396)
ANQLTVVNTLQSDAVKEKFEAVMGEKANGFVSSVLSVVTNNNILS
KADENSVYTSAMKAAVLDLPVEPSLGMAHIVPYKGKAQFQIGYKG
LIQLALRSGQVVGLNAGKVYEGQFKSFNALTEKLDIVDIYNPKKD
EPIVGYFAYMKLSNGFEKTTYWTKEQVEEHGKKYSQSYDSKFSPW
QTNFDAMARKTVLKSILSTYAPLTIEMQNANDFDNGKNTGIEPLE
VKDVTPETDNESLLTDLLEDEPSVNTETGEIIEDTELDLDYGQIN
AK
WP_006845711.1 RecT
[Weissella koreensis]
(SEQ ID NO: 397)
ANELVKQLKSEKVAAQFETTAGKNAAAFASEVAISVMGNKALENA
SLSSVVVEATKASALGLSLLPTVGEAYLVPYKGQAQFQLGYKGLV
QLAMRSGQMKSFGTVKVYEGEHPRWDKYSQELHTDGDETGEVVGY
YAQFTLINGFKKADYWTKSAVEEHRSRFSKSKSGPWSTDEDAMAQ
KTVLKSILQYAPKSSEMTRAMASEDMNGDISEGTAKPIDITPETE
TPKVEEANQNQQIDTNEMVDEIKEYAKETNEAPKEQTVSAADEFF
K
WP_073846185.1 RecT
[Amycolatopsis] sp. CB00013
(SEQ ID NO: 398)
TTQTVTSAVAQQDSSPAALVRKYRTDFATVLPSHIKPETWLRIAT
GALRRSSQLAHAAEKNPTSLLVALLDAARKGLEPGTEQYYLVPRK
TKRGPEVLGITGYQGEVELMYRAGAVSSVKVEGVREHDTFAYNPG
EHDRPVHEINWRANRGDLVLAYAYAYAKMRDGATSNVSVLSADDI
AVILSKAEGADSPFSPWQWNPKAMWLKSAAHQLAKWVPTSAERVW
QPDGPPLEAPPATPVTLPTVEDVVDAEVVEDWPTTPADTADSEQ
WP_142511229.1 RecT
[Leuconostoc pseudomesenteroides]
(SEQ ID NO: 399)
ANEITLAKQLSSDKVVEQFAATAGESAKSFAKEVALTISGNPALQ
HAKLGTVIVEATKASALGLSLLPTVGEAYLVPYKGDAQFQLGYKG
IVQLAMRSGQMKSFGAESVYEGENPKWDKYNQELVTDGEETGKII
GYYAFFTLVNGFKMAAYWPKEKVEAHRDRFSKSKKGPWSTDFDAM
AKKTVLKSILQYAPKSSEMKRALAEDTQAEYVQAGIQDVTPEPAN
IEAPIETANAPEINAQEESLFGELSDVDKETAPNPFAQNLGGDN
WP_023055804.1 RecT
[Peptoniphilus] sp. BV3C26
(SEQ ID NO: 400)
TNIQKQENRALSPVNQMKNLLANQGMQNLFADALKENKDRFIASI
IDLYNGDNYLQNCDPKEVAMEALKAATLNLPINKSLGYAYIVPFK
NKGKLTPQFQIGYKGYIQMAQRSGQYKALNAGIMYEGMEIKRDFL
RGTFEIVGEPKSDKAIGYFAYFQLLNGYEKALYMSKEDITDHAKR
YSQSFGSDFSPWKNQFDEMAQKTVLRRLLTKYGVLTTEFQEAAKR
EEDEEVLKATEENAMIEMNSQEETIAVDPKTGEIIEETEAPF
PCR98661.1 RecT
[Lactococcus fujiensis] JCM 16395
(SEQ ID NO: 401)
KSAPVQARFQEVLGKKSSGFVSSLLTVVNNNNLLKRATPDSIMTA
AMKAATLDLPIEPSLGFAYIIPYGQEAQFQIGYKGLIQLALRSGQ
ITGLNSGIVYKSQFISYDPLFEELEIDFMQPEDEVVGYFASMKLS
NGFMKVVYWTKARVENHKKRFSKAGAKSPWATDFDAMAQKTVLKA
MISKFAPLSQEMQIAVIADNESETLEPKDVTPEQPLISIDEPKEN
ENSQSQISIPEDQAPQQENEEFVEELFPVGQA
WP_106316803.1 RecT
[Actinoplanes italicus]
(SEQ ID NO: 402)
PETIANAVAQRDQSPTALVADYRNDFAAVLPSHLPPATFVRLAQG
VLRRDQNLMRTAMNNPGSLMTAMLDCARLGHEPGTPAYYFVPIKG
AIEGWEGYRGVIERIYRAGAVQSVRAEVVRENDFYEYEEGMPHPI
HRYERFASPEQRGPLLGVWAYAVMLDGGMSRPVEMGREEVLAHRD
MNPSNNRSDSPWKKWERSMWLKCAVHELEKWVPSSTEYRREIARM
SAPQPAAAAAPVTYVPPQVGQRDAIEGEVAEDWPEPAEVPGGAQ
WP_013655830.1 RecT
[Cellulosilyticum lentocellum]
(SEQ ID NO: 403)
SDKKELVLKETHSRLNQLLATKMEAMPKDFNQTRFLQNCMTVLQD
TKGIENCHPVSIARTLLKGAFLGLDFFQRECYAIPYGGELQFQTD
YKGETKMAKKYSIRDIKDIYAKVVRKGDEFKEEIVAGQQVVDFKP
LPFNDAEIIGAFAVVLYQDGGMEYETMSTKQIEGIRDNFSKMKNG
LMWTKTPEEAYKKTVLRRLTKKIEKDFASIDQAKAYEESSDMQFK
QDEQKQDAKDPFADAVDVEFTEETEGQVRLDGEADGAK
WP_148001988.1 RecT
[Streptomyces] sp. adm13(2018)
(SEQ ID NO: 404)
SQIGNEIARQSHSPAAIIEQHKADLAVVAASHVRVDTFARLAVGV
LRQNEKLAAAAANNPGSLMSALMTAARLGLEPGTEQFYLRPIKRK
GQLEVQGIVGYQGIVELIYNAGAAQSVVVEVVRARDEFAWTPGAL
DEHRPPRWPGAMKQPHHKVDWFGDRGPLVGAYAYAVMQGGAISKV
VVLNRDHIARAKAKSDGADTDYSPWRTDEEAMWLKTAARRLGKWV
PTSAEKRTGVIERLDTPPAPLNEIDPDEDDEPIDGELVD
WP_011988985.1 RecT
[Clostridium kluyveri]
(SEQ ID NO: 405)
PDKKMMVLSESHKALNKLLETKKEALPKDFNKSRFLQNCMTVLQD
TKDIDKCQPISVARTMLKGAFLGLDFFNRECYAIPYNGNLQFQTD
YKGEIKLAKKYSINPIKDIYAKVVRKGDEFQESIVNGHQTVNFKP
LPFNNDEIIGAFAVCLFQDGSMIYETMTKQEIEDIRNNFSKAKNS
PAWVKTPGEMYKKTVLRRLCKFIELDFNSIESKKTYDEASDFQFE
HEPNKEVSNFDKGSIDEDKTVEADTETEAKEDNREYAFKESE
GAC42786.1 recombinational DNA repair protein
[Paenibacillus popilliae ATCC 14706]
(SEQ ID NO: 406)
STSHLLTIHNNLEKLIDSKREAMPKSFNKTRFLQNCMTVLQDTKD
VGKCDPQSVARTLLKGAFLGLDFFNKECYAITYGGSVQFQTDYKG
EKKLAKKYSVRPVKDIYAKLVREGDEFIEEIKDGQPTVQFKPLPF
NDSEIKGAFAVSLFEDDGLAYEVMSVAEIELTRKNYSKQPNGQAW
VKSKGEMYKKTVLRRLCKNIELDFDTIEQAQAFEDSSDFEFNKEP
KQAQQSPLNPQATVIDAEYEEVKEESDNETNQE
OBR91022.1 RecT
[Clostridium ragsdalei P11]
(SEQ ID NO: 407)
LDKQANGFITSLLNLKQDKLKGCNDMTVLGSALKAAPLKLPIDPN
LGFAWIIPFKNHGKLEAQFQVGYRGFIQMAQRSAQYKKLNVTEIY
EGQLKSFNPLTEELELDLDNKQSDEVVGYAAYFRRLNGFEKMVYW
SKEKVTAHARRFSKSFGNGPWKTDFDAMARKTVLKNMLSTWGILS
IDMQEAITSDSKIIKTTEDDYELLEEGTEDESNANVTDVEYTESD
ESGKEEDGKDPYEGTPFSENNTES
SEI77195.1 RecT
[Paenibacillus polymyxa]
(SEQ ID NO: 408)
PDKLLVIHDNLNKMLDEKSEAMPTSFNKTRFLQNCMAVLQDTKDI
EQCDAKSVARTMLKGAFLGLDFFNKECYAIPYNENIGTKNKPRWI
KSLQFQTDYKGEKKLAKKYSTRRVKDIYAKLVRDGDDFREEIESG
QPTINFRPLPFNDGIIRGAFAVALFEDGGMIYETMSLKEIEKTRD
DYSKQSTGKAWTKSPGEMYKKTVLRRLCKNIELDFDTIEQAQAFE
DSSDFDMNKELKPQQQSPLNPNTTIIDAEYEEIKEEPADGPEQE
KKT72154.1 RecT
[Candidatus Collierbacteria bacterium
GW2011_GWB1_44_6]
(SEQ ID NO: 409)
SNQIQIKSEVDLKMILANQYMKQINNFFGNEKQAMKFLSSVMSAV
QRIPELLNCEPKSLINSFMTMAQLGLMPSEVSGEAYVLPYNNKNG
KVAQFQLGYQGLVTLFFRAGGQKIRAEIVRKNDEVSYVNGEIKHT
IDIFKSNEERGEAVGAYAVATINGQEVCKYMNATDILAFGSRFSK
SWTTSFTPWKEANDPELNMWKKTVLKQLGKMLPKNESINLAIAED
NKDSIISDRLLPAVEESKNLTMGSIVKTEEPVIEVEPEEIKQ
WP_125777163.1 RecT
[Antribacter gilvus]
(SEQ ID NO: 410)
SADVVIRQHATELTSVLPSHLAEKGDGWLNAAVAAVRKDRNLWNA
ANSDPGAVMNALAEAARLGLQPGSKEYYLTVRGGKVLGIVGYQGE
IELMYRAGAVSSVIVEPVFERDGFEYTPGVDDRPKHRIDWDADDR
GPIRLAYAYAVMKDGAVSKVVVVNKTRIRRAKDASATAGKSHSPW
TSDEVAMWMKTAAHDLAKWVPTSAEYIREQLRAVKEVEAEPARAS
DPRPEPVHIVEAQILDEDPFPNAPEDGAA
WP_130123223.1 RecT
[Lactococcus] sp. S-13
(SEQ ID NO: 411)
SNQITKTQQTLKSPEVKAKFEEVLGKKADGFVASLLSVVGNSNLK
TVEANSVMTAAMKAATLDLPIEPSLGFAYVIPYGREAQFQIGYKG
FIQLALRSGQLTGLNCGIVYESQFVSYDPLFEELELDFTQQASGD
AVGYFASMKLANGFKKVTYWSKEQVLAHKKKFVKSANGPWRDHFD
AMAQKTVLKAMLTKYAPASIESKMIQTAITEDDSERFENAKDVTP
DEPVISIDEPVTSEVSQNESSAESQEQFPEDEVEELFPIGKS
WP_147265819.1 RecT
[Nocardia puris]
(SEQ ID NO: 412)
AESISSEVARQASPLAVVARYRSELAGSLPAAVRHDVDRWLMVAE
MAVRRSPDLMEIVRRDQGASLMRALIECARLGHEPGSPEFYLIPR
GGIVSGEESYRGIIKRILNSGEYQRVVARVVHERDRFSFDPRIDE
IPDHRPAEGERGAPARAYAFAVRWDGTPSTVGEATPERIIAAKAK
ARGVDRKDSPWNSPTGVMYRKTAIRELASYVHTSAEPRPRPAAPT
EPPAVDEVSTVYDAEVIDEVDVLDITAEPTA
TCP18101.1 RecT
[Nicoletella semolina]
(SEQ ID NO: 413)
LKNADPQSVFNAACMAATLNLPIQNGLGFAYIVPYQNKKEKKTEA
QFQLGYKGLIQLAQRSGQFKRLVAVPVYEKQLIAEDPINGFEFDW
KQKPENGEKPIGYYAYFKLLNDFTAELYMTTHEVDEHAQRYSQTY
RTYLDKKSKGQWASSVWADNFEAMALKTVMKLLLSKQAPLSVEMQ
QAVLADQAVVKNVETNEFSYVDNQIEEAEYTELKVSTDIFEKCKQ
SILNKETTLQELCDSGYEFSQEQYAELEKLEVE
OAB27843.1 recombinase
[Paenibacillus macquariensis] subsp. defensor
(SEQ ID NO: 414)
SDKLLVIHKNLENLLDSKREAMPSNFNKTRFLQNCMTVLQDTRDI
DKCDATSVARTMLKGAFLGLDFFNKECYAITYAGAVQFQTDYKGE
KKLAKKYSVRPVRDIYAKLVKEGDDFKEEVKDGQQTIQFAPKPFN
DGEVLGAFAVALFEDGGLVYEVMSKVDIETTRKNYSKQANGQAWT
KSPGEMYKKTVLRRLCKNIELDFDTIEQAQAFEDSGDMDLNKEVK
PPQVSPLNATVIDAEYTEIREGDPNATNQE
WP_019417330.1 RecT
[Anoxybacillus]
(SEQ ID NO: 415)
ATTQSLKNQIAKKQNSNIQQGVTLKQLLNSESMKKRFEEVLGKRA
QQFATSILNLYNSEKMLQKCEPMSIISSAMVAASLDLPVDKNLGY
MYIVPYGTTATPIMGYRGYIQLALRTGQYKHINVIEVYEGELQKW
DRLTEEFEMDSKQKKSDVVVGYAAYFELINGFRKTVYWTREQIEA
HRKKYSKSDFGWKNDFDAMAKKTVLKSLLSKWGILSIEMQNAFNE
DEKEVDTKEVKDITSEVQEAEYIEAEAFEVPIETETPQQEEIVFD
AQ
CDA71469.1 phage RecT family
[Ruminococcus] sp. CAG: 579
(SEQ ID NO: 416)
NERTNLQYAPAPVERFKECLNSHEIKARLKNSLKNNWTQFQTSML
DLYSGDAYLQKCDPMAVALECVKAATLDLPISKSLGFAYVVPYNN
VPTFTLGYKGLIQLAQRTGQYRTINADVVYEGEIRGADKLSGMVD
LSGERTGDEVVGYFAYFKLINGFEKMIYMTRAEAEKWRDDYSPSA
KSKYSPWRTDFDKMALKTCIRRLISKYGIMSVEMQGVMTEEAEPR
AAAAAKRAEETVQANANSKVIDIDAAPPAANESPAEAAPQPDF
WP_019108121.1 RecT
[Peptoniphilus senegalensis]
(SEQ ID NO: 417)
TNQIARKPVNEIKNVLSVPSVRNLFDNALADNAGAFVSSLIDLYG
GDSYLQNCEPKDVVMEALKAATLKLPINKNLGFGYVVPFKNKNGK
LVPTFIIGYKGLIQLAMRTGQYKAINSGIIYEGMEIKEDVLRGTL
EIKGSKQSEKIKGYFAYFQLINGFEKALYMDVEEAADWGRKYSKS
FAKGPWTTEFDAQAQKTCLRRLLSKYGVLSTEMQRLEKTEEDVDI
AVGTIENNAVEELNIPSSQADYIVDEETGEILDDEEIVAPF
AFH22576.1 RecT family protein
[environmental Halophage eHP-30]
(SEQ ID NO: 418)
TEQNQTPAKTESKSPIKAQLYKDNVQQRFQELLGERASAFMTSVM
SVVKDNDQLSQAEPSSVLNAAMTAATLDMPIDNNLGMAYIVPYKD
GKSGKTYAQFQLGYKGFIQLAQRSGQFKTISATPVRQGQIVTADP
LRGYEFDFTQGQDKEVVGYAAYFALLNGFEKTLYMSKAEMEQHAA
SYAAGYKKGYSNWNRKFDEMALKTVIKQLLSKYAPLSVDMQKAQQ
TDQTVSVEEPNAIEQQEAAPEIDASSNNNQNQ
WP_138067957.1 RecT
[Streptococcus pseudoporcinus]
(SEQ ID NO: 419)
ANQLTVVNTLQSDAVKEKFEAVMGEKANGFVSSVLSVVTNNNLLA
KADFNSVYTSAMKAAVLDLPVEPSLGMAYIVPYKGKAQFQIGYKG
LIQLAQRSGKVTKLNSGKIYKGQFKSYNALSEELDIDDIYTPKED
EEVVGYFGYMKLSNGFEKITYWTKERVEKHGKKYSQSYDSKFSPW
QTNFDAMAEKTVLKSILSTYAPLTIEMQNANDFDNGKNTGIEPLE
VKDVTPENDNESLLSDLLEDEPSVDAETGEIMENTELDLDYGSIN
AK
WP_072904346.1 RecT
[Hathewaya proteolytica]
(SEQ ID NO: 420)
ADSKKELILKESYSVLDRLIETKISAMPKDFNRTRFLQNCMTVLQ
DTKDIEKCQPISVARTLLKGAFLGLDFFQKECYAIPYGGTLQFQT
DYKGETKMAKKYSIRPIKDIYAKVVREDDLFEEEIKEGQQFVNFK
PIPFSDKPIIGAFAVVLYQDGGMEYETMSKTQIEGIRDNFSKMKN
GLMWTKTPEEAYKKTVLRRLTKKIEKDFDTIEQAKTYEETSDSEF
KKEEKCNEKSVFDVEYSEVESEELEQQTMLENSPFGGEQ
GAE17732.1 RecT
[Bacteroides pyogenes]
DSM 20611 = JCM 6294
(SEQ ID NO: 421)
QVADPQSVLNSAVIAATLDLPINPNLGFAAIVPYNDRKSGKCIAQ
FQLMYKGLVELCLRSGQFASLIDEVVYEGQIVKKNKFTGEYIFDE
DAKTSNKVIGYMAYFRLVNGFEKTFYMTSEEVTAHAKAYSQSFKS
GYGVWKDNFDIMARKTVLKLLLSKYAPKSIEMQRAITFDQAAVKG
DLTETNVDEAEIEYIDNESGSDKIKQAAEDAVIQSQQKTLL
CDF09406.1
[Eubacterium] sp. CAG: 76
(SEQ ID NO: 422)
AERKQITTKEYLAEVKGGLENELNLNAKALPENFNQSRFVLNCIS
LIKSNLSNYNNITPESVYLALAKGAYLGLDFFNGECYAIPYSGEV
NFQTDYKGEIKLAKTYSRNPIKDIYAKNVRDGDFFEEIIESGKQS
VNFRPVPFSDKKIIGTFAVVLFKDGSMMYDTMSVKEIEEVRNNFS
KAKNSKAWAATPGEMYKKTVLRRLCKLIDLDFNSQQRLAYEDAGD
FDKEKADEPVADDTVNVFDAEFKEVEPENKDAAIIEEMGLEEA
WP_099299656.1 RecT
[Pediococcus pentosaceus]
(SEQ ID NO: 423)
MNDISKVPMKVLVQQDKVQRMLENTLKGKTRQFTTSLINVVNSNQ
SLADVDQMSVIKSAMVAASLDLPIDQNLGFMWLVPYKGMATPQIG
YKGYIQLALRTGQYKKLNTIVVHEGEMKYWNPLTEDFEYDPKGKE
SDEVIGYLGYLRMINGFEKTVYWTKQNIEDHRMKFSKMSGKAKPS
GVWASNYDAMALKTVMRNLLSKWGIMSIEMQQAVVQDEKAPETDV
RDVTPTETNSIDSLLAPEPKGEPINDSNEATVPTNAE
WP_118227047.1 RecT
[Bacteroides eggerthii]
(SEQ ID NO: 424)
GTVTTVPQLKSMLANENVKSRFKEILGKKAPGFISSIVAVANSNT
LLQKAEPQSIMNAAVIAATLDLPINPNLGFAYIIPYGNQASFQIG
YKGMTQLAMRSGQYKTINVTEVYEGEIKSENRFTGEYTFGERKSD
KIVGYMAYFSLTNGFEKYMYMSREECEKHGKKFSQTYKRGGGLWA
TDFDSMSKKTVLKMLISKYGILSIDMQRAQTFDQAVVKDDLVEKN
IDEAEVSYEDNPTNADVRRNAMKEALEEAEVVDETTGEIFNQPAQ
WP_094754495.1 RecT
[Criibacterium bergeronii]
(SEQ ID NO: 425)
EVNNMNNQMQQTATQVTPINQMKNLLANKGINQMFEQALKMNAGA
FISSLIDLYNSDGYLQKCEPKDVAMEALKAATLNLPINKGLGFAY
IVPYGKAPQFQIGYKGYIQLAMRTGQYKHINAGAVYEGEEVKENR
LAGTVEILGDKKNDNETGYFAYFKLTNGFEKCLYMSKQEMTTHAQ
RFSKAFKNGPWQSDFSAMATKTVLRLLLSKYGVLSTQMQEAIAKE
NDDELQQQINQNANKEVIDIEKIDNKNVIDIEAIDAADDDIEAPF
WP_045553720.1 RecT
[Listeria]
(multispecies)
(SEQ ID NO: 426)
ATNDELKNQLANKQNGGQVASAQSLDLKGLLEAPTMRKKFEKVLD
KKAPQFLTSLLNLYNGDDYLQKTDPMTVVTSAMVAATLDLPIDKN
LGYAWIVPYKGRAQFQLGYKGYIQLALRSGQYKSINVIEVREGEL
LKWNRLTEEIELDLDNNTSEKVVGYCGYFQLINGFEKTVYWTRKE
IEAHKQKFSKSDFGWKKDYDAMAKKTVLRNMLSKWGILSIDMQTA
VTEDEAEPRERKDVTEDESIPDIIDAPITPSDTLEAGSEVQGSMI
WP_106024518.1 RecT
[Clostridium thermopalmarium]
(SEQ ID NO: 427)
ATVNELKNEIATKKETGVGSAGNTIKGLINSPAIKKRFEDVLNKK
APQYMSSIVNLVNGDTNLKKCDQMSVIASCMVAATLDLPIDKNLG
YAWIVPYGNRAQFQLGYKGYVQLALRTGQYKAINVIEVHEGELIE
WNPLTEELKIDFSQKKSDAIIGYAGYFELLNGFKKSTYWTKEQII
RHKNKFSKSDFGWKKDFDAMAKKTVLRNMLSKWGILSIEMQNAYT
ADQATIRPEAVETGDIKGNVDYVEADFEENYEGTPFEEVEEGGVN
E
WP_073010654.1 RecT
[Virgibacillus chiguensis]
(SEQ ID NO: 428)
ATNSSLKNQIANKGNGNQNTPQGYTVKQLMSASSVKNRFEETLGK
KAPQFMASVINLVNGDTNLQKCDQMSVVSSAMVAAALDLPIDKNL
GYAWVVPYGNKATFQMGYKGYIQLALRTGQYKNINVIEVYEGEVK
SFNRLTEEIELEFEGKESDKVIGYVGYFELINGFRKTVYWSKDEI
ERHKKRFSKTGFAWKDNYDAMAKKTVIRNMLNKWGILSIDMQTAV
TTDGNAVTQDFEQEDSGLVIDAEFSEVNEASEGQQEIKFENADA
WP_111921306.1 RecT
[Clostridium cochlearium]
(SEQ ID NO: 429)
ATNESLKNQLATKKETGIGSAGNTIKSLINSPVIKKRFEEVLDKR
APQYMSSIVNLVNSDTNLKKCDQMSVIASCMVAATMDLPVDKNLG
YAWIVPYGNKAQFQMGYKGYVQLALRTGQYKSINVIEVHEGELEE
WNPLTEELKIDFSKKESDAVIGYAGYFELLNGFKKSTYWTKEQIT
KHKNKFSKSDFGWKKDFDAMAKKTVLRNMLSKWGILSIEMQNAYT
ADHGVIKNEIMETGEVKENVEYIEADFESYEGTSIEEGGSNE
WP_019125538.1 RecT
[Peptoniphilus grossensis]
(SEQ ID NO: 430)
TNIQKQENRALSPVNQMKNLLKNQGMQNLFADALKENKDRFLASI
IDLYNGDTSLQDCNPKEVVMEALKAATLNLPINKNLGYAYIVPYN
SKGTTRPQFQIGYKGYIQMAQRSGQYKALNAGILYEGMEVKRDFL
RGTFEIIGEPKSDKVMGYFAYFQLLNGYEKAIYMTKDEVTEHAER
YSQSYGSKYSPWKKQFDEMGQKTVIKKLLSKYGVLTTEFQDAVKE
EEDREVLRATENNAMLEMTNPDEEEETIEVNPETGEIIEDDVKAP
F
ERL63827.1 YqaK
[Schleiferilactobacillus shenzhenensis] LY-73
(SEQ ID NO: 431)
SAVSESKDLQHVDQLSVLNSAMTAASLNLPINQNLGFFYLVPYKG
IAQAQMGYKGYIQLAQRSGQYQRLNAIPVYADEFGSWNPLTEELD
YTPHFEDRKASDKPVGYVGFFKLANGFEKTVYWSRKQIEAHRDRF
SKSSKSSASPWNTDFDAMALKTVLRNLITKWGPMTTDIQRANDAD
EGDYKNDLSTDTSEPKDVTPGASLEQFLGETDQQQKPATKPAPKK
KAEEAKPNDLKPDVTHDPNEHTEQTSLSDDDLPFD
WP_051267408.1 RecT
[Gulosibacter molinativorax]
(SEQ ID NO: 432)
TDLTEKIATKAVAVKKDPKIADLMKSYEPQFARSLGKSMDAAKFG
QDALTAIKQTPKLLEADQRSLFGAIFLAAQLKLPVGGPLAQFHLT
PRKVAGEMTVVPIIGYNGYIQLAMNTGLYSKVGAFTVHANDHFRT
GANSERGEFYDYERATGDRGELTGVIGYAKVKGFDESSFVYLDAA
TVRERHRPKFWDKTPWASDEGEMERKTAIRVLQKYLPKSIEAAPL
ALAAQADQATVRRVDGVDDLQIDHEDIAIAEVIEDD
WP_112330076.1 RecT
[Cereibacter johrii]
(SEQ ID NO: 433)
TENTAQAPAAARQLTPIQAISQTLESDAFAPKISASLDGTGISPA
RFKRAALACLSRPEASYLVEKCDRGSIFTAVMNAAAAELELHPAL
GQAYIVPRGGQAVLQVGYKGFIALASRAGLAVEADVIYAGDRESI
RKGTNPDVSVEPELDPAKRGEWVAVYVITHYASGAKTLTFMTRAE
VEAIRNRYSDAYKRGGAGAKTWNESPEEMAKKTCIRRASKLWPIS
VPGGGDDDGGEVIEADPAPVPAPRMRDVTPGGGLDRLAASL
WP_063601171.1 RecT
[Clostridium coskatii]
(SEQ ID NO: 434)
SDKKMVVLNESHTMLNKLLETKQEALPKDFNKARFLQNCMTVLQD
TKGIEQCQPITVARTMLKGAFLGLDFFNKECYAIPYKDNLQFQTD
YKGEIKLAKKYSFNPIKDIYAKIVRQGDDFQEAIINGQQTINFTP
VPFNNGEIIGAFAVCLFQDGSMLYETMAKQEIENTRKNFSKAPNS
PAWTKTPGEMYKKTVLRRLCKLIELDFDSVECKKVYNETSDFEFE
NQQHEVSNFDKKDIDEDKIVEADVEVQDDNENNVPEDGE
WP_118206945.1 RecT
[Bacteroides stercoris]
(SEQ ID NO: 435)
STITTIPQLKSMLANDNVKARFKEILGKKAPGFISSIVAVANSNT
LLQKAEPQSIMNAAVVAATLDLPINPNLGFAYVVPYGNQAQFQMG
WRGFVQLAMRSGQYKTINVNEIYEGEIKKSNRFTGEYEFGERASD
KIVGYMAYFSLINGFEKFLYMSKEDCEKHGRKFSQTYKRGTGIWS
TDFDSMAKKTVLKMLLSKFGILSIEMQRAQTFDQAIIKDNLAETD
IDEAEVSYNDNPDNEEARRNAMKEALQEAEVVDENTGELFNTETK
WP_099840029.1 RecT
[Clostridium combesii]
(SEQ ID NO: 436)
ANTKAIVLQETANNLNTLLKAKVKALPKGFNETRFLQNCMTVLQD
TRNIEKCNSVSVARTMLKGAFLGLDFFSKECYAIPYNDYKTGKCH
LEFQTDYKGERKLMKQYSVRPIKDIYAKVVREGDKFEEIIEKGIP
TINFRPKPFSNEKIIGVFAVVLFEDGGLLYETMSVEDVEKIKVGF
AKRDKEGNYSKAWTATPEEMYKKTVIRRLRKSVELEFDSVEQQKT
YEEASEFDVKRDEEVKEEASPFENVDFEEAEEGNTIEAKQE
WP_069686512.1 RecT
[Oceanobacillus sp. E9]
(SEQ ID NO: 437)
ATNDSLKNQLSSKQGNQNTPSGYTIKQLMGAESVKKRFEEMLDSK
ASQFMASVINLVNGDTNLQKCDQMSVVSSAMVAATLDLPIDRNLG
YAWVIPYGNQATFQLGYKGYIQLALRSGQYRNINVIEVYEGELQS
FNRLTEEIELDFEKRTSDKVIGYTGFFELINGFRKTVYWSKAEIE
KHKNKFSKSGFGWKNDWDAMAKKTVVRNMLNKWGILSIDMQKAYV
EETKDPSEPNGEVIDLNLTEDELTAAQEQFSDENANE
RMD50745.1
[Candidatus Parcubacteria bacterium]
(SEQ ID NO: 438)
TEYKRPQQPEQTKMLSAKLNQAGAPNKVSSFDVQLRDWFKKHSRK
MQTLAGSKEEANKIITSLIFVAQRNPKLMTCTMESIGECLMQSAQ
LKLYPGPLQECAYVPFGNRATFMPQYQGLCKLAYNSGVVRSIATE
VVYANDLFEFELGTNAYLRHVPTLSDNRGERIAAWCVVKTTHGEV
IIVKPISFIEGIRKRSPAGNKKDSPWNTSDDDYDAMARKTVLKQA
LKTIPKSSDLAAAIQVDNAVESGSVDNVVTHIEPVTDPTPEETKE
WP_061413958.1 RecT
[Lactococcus sp. DD01]
(SEQ ID NO: 439)
ANLTPTQTVLKSDAAKRKFEEVLGKKTNGFVGSLLSLVGSTNLKN
VDSNSVMTAAMKAATLDLPIEPSLGFAYVIPYGREAQFQIGYKGL
IQLAIRSGQVTKLNAGPVYENQFIKYDSLFEELEIDFSMPQGVEI
AGYFASMELANGFRKIIYWDKEKVTAHGKRFSKSFNRSSSPWQTD
FDAMATKTVLKAMLSTYAPLSTEMQQAIVADNESATPKDATPVTD
DLVLEAVEDSKQIEENEIINDQVASENYQEPQGEPEVLDLEL
WP_147129628.1 RecT
[Nocardia ninae]
(SEQ ID NO: 440)
AESISKEVARQANPLAVVAKYQNELGKSMPAAIRGDVGRWMMVAE
MAVRKNPKLLSIVQADQGASLMRALIECARLGHEPGTKYFYLVPR
GNQISGEEGYHGIIKRVLNSGHYQKVLARTVFERDEYSFDPLTDQ
LPTHVPASGERGKPVSAYAFALHWDGTPSTVAEASPERIAAAKAK
SYGTDRKDSPWQSVTGVMYRKTAIRELEPYVHTSAEPQPRQDNAG
SRGAVMDPSTYDDAEPLDADVLDITADQIAEHDGEGAL
WP_074846740.1 RecT
[Clostridium cadaveris]
(SEQ ID NO: 441)
ATNSSLKNQLSKKENVTIGNTMQGLLNNPKMKKRFEEILDKKAPQ
YMSSILNLYNGDTSLQKCEPMSVLSSSMIAATMDLPVDKNLGYAW
IVPYKNKAQFQMGYKGYIQLALRTGQYKHINAIEIHEGELVNWNP
LTEELEIDFTKKESDKIIGYAGYFELLNGFKKSTYWTKTQIENHR
KKFSKSDYGWNKDFDAMAIKTVIRNMLSKWGILSIEMQNAYTADE
NIIKDSFIDDSENVSANIEDLVEADYTVNQDSLESKEEFEGTPLE
WP_038246219.1 RecT
[Virgibacillus]
(multispecies)
(SEQ ID NO: 442)
ATNDSVKNQIANKNQGSNQVNPNNLGLKQLLSTPTMRKKFDEVLD
KKAPQFMSSLLNLYSNDSYLQKAEPMSVVTSALVAATLDLPIDKN
LGYAWIVPYGGKAQFQLGYKGYIQLALRTGQYRNINVIEVYEGEL
KSFNRLTEEMELDFEQKQSDKVIGYTGYFELINGFRKTVYWSKEE
IEKHKKRFSKSDFGWKKDWDAMAKKTVIRNMLNKWGILSIDMQKG
IVEDNKDPIEKANEFDEQDIIEADFSEVNDDQEIDFSDAQ
WP_106064284.1 RecT
[Clostridium liquoris]
(SEQ ID NO: 443)
TTASELKNQLATRKETGVGSAGNTVKGLLESPAIKKRFEEVLKQR
APQYMSSIVNLVNGDANLKKCDQMSVIASCMVAATLDLPVDKNLG
YAWVVPYGNKAQFQLGYKGYVQLALRTGQYKSINVIEIHEGELIE
WNPLTEELRIDFEKKKSDAIIGYAGYFELINGFRKSTYWTKEQIT
KHKNKFSKSDFGWKKDFDAMAKKTVLRNMLSKWGILSIEMQNAYT
ADQETIKSEVLETGNIKENVEYVEADFDVDFEGTPFEEGVTNE
WP_028562280.1 RecT
[Paenibacillus pinihumi]
(SEQ ID NO: 444)
ADANKLLVINEKLIKLIESKQDAMPKSFNKTRFIQNCMAVLQDTD
EIDKCDATSVARTLLKGAFLGLDFMNKECYPIIYGGKCTFQTDYK
GEIKLAQKYSVRPVLNIYAELVREGDFFLKEVKDGQRTIQHKPPE
GFNDGKVIGAFAIVLYKDGGMDCESMSVAEIETTRKNYSKQANGP
SWTKSPGEMQKKTVLRRLCKTIQLDFDTIEAKEAFEDGGDFDFKQ
DPKPQQQSPFDKNATVVDAEYEEVEEEDQSESAT
WP_068672306.1 RecT
[Oceanobacillus sp. Castelsardo]
(SEQ ID NO: 445)
ATNSTLKNQISNKKQGNNQVGKTQGTTMKQLLASPAVMNRFEEVL
GKRANQFTASILGLYNSEKMLQKAEPMSVISSAMIAATLDLPVDK
NLGYAWIVPYGGKAQFQMGYKGYIQLALRTGQYRNINVIEVYEGE
LKKWDRLTEEIELDFESRTSDKVIGYTGYFELINGFRKTVYWSKE
DVEKHKKRFSKSDFGWKNDWDAMARKTVIRNMLNKWGILSIDMQK
GMVEDSKDPVEVNEEFSSDVIDADYEVVGENEQQDFTVEENA
WP_067592792.1 RecT
[Nocardia terpenica]
(SEQ ID NO: 446)
SSIAAAAESAEVTPASIINKYRDDIATVLPPKLRERIDRWIRLAI
GAVNSNPELISRVRADQGASMMQALMKCAALGHEPGSGLFHLVPK
GSRIEGWEDYKGILQRIDRSGVYARTVIGVVYANDEYSYDQNVDE
RPRHVRATGDRGEPISSYAYAVYPSGAITTVAEATPEQIASSKSK
ARGADNAASPWRAPGAPMHRKVAVRLLEKHVATSAEDRREPISRS
AANDVVIDATADYYQEP
WP_079708113.1 RecT
[Paraliobacillus ryukyuensis]
(SEQ ID NO: 447)
ATNDTLKNQISNKKNNQVAEGKQGTTMKGLLNSPAVMKRFEEVLG
KRANQFTASILSLYNNEKTLQKSEPMSVISSAMIAATLDLPIDKN
LGYAWIVPYGNKAQFQLGYKGYIQLALRTGQYRNINVIEVYEGEL
VKWNRLTEELELDFEQKKSDKVIGYTGYFELINGFRKTVYWSKAD
IEKHKQKFSKSNFGWSNDWDAMAKKTVIRNMLNKWGILSIDMQKA
YSTDEIEQEQESNDFIDGEWAEVSEDDITEAMNEV
OLA20462.1 BHW17_09115
[Dorea sp. 42_8]
(SEQ ID NO: 448)
AVNNSLAKRDQSMKLSVYLQNDAVKKQINQVVGGKNGTRFISSIV
SAVQSTPALQECTSPSIVNAALLGEALNLSPSPQLGQFYMVPFDN
RKKGCKEAQFQLGYKGYIQLAERSGYYKKLNVLAIKEGELIRYDP
LDEEIEVELIDDDVIREETPAMGYYAMFEYENGFCIQQKWRSEDF
GTFRAGQNSGKGSLEVFFFLVQRF
WP_058906805.1 RecT
[Lactiplantibacillus plantarum]
(SEQ ID NO: 449)
SNELAHMPMKQLVKQDAIQQMLSRTLADKASQFSTSLINLVNGNQ
SLAKVDQMSVIQSAMVAATLNLPIDQNLGYMWLVPYKGRATPQIG
YKGYIQLAQRTGQYLAMNAIAIHSGELKGWNPLTEDFQFDPMGRT
SDEVIGYVGYFKLTNGFEKTVFWTKASMEEHRMSFSKMSGGKTPQ
GVWASNYDAMAIKTVLRNMLSKWGPMSIEMEQALANDETAPQTPL
NVEAEESASETTDNMLDKFRQQQGEVNTSDQEHNTEDQGDPRDQS
RZT66774.1 RecT
[Leucobacter luti]
(SEQ ID NO: 450)
SDLSQAAVAVKKSKTVEDYLTEYEPQFQRALGKSMDAAKFSQDAL
TAIKQTPQLGQADLQTLFGSLFLAAQLKLPVGGPLAQFHLTPRKR
GDKLEVLPIIGFGGYVQLIMNTGLYSKVGAFLIYEKDYFDEGANS
ERGEFYDFKKSRGDRGPVVGVIAYVKLKGFDESQYVFLDADTIRS
RHRPRYWEKTPWGSDEGEMFKKTGVRVLQKLLPKSVEAAPLALAA
DADQATVRKVDGIEDLTIQHDVVDAEVVPDGVPV
WP_087916041.1 RecT
[Paenibacillus donghaensis]
(SEQ ID NO: 451)
SNTQLATIHNNLERLIDSKRDAMPSSFNKTRFLQNCMTVLQDTYG
IEKADPVSIARTMLKGAFLGLDFFNKECYAIIYGGKVEFMTDYKG
EVKLAKKYSIKRIKDIYAKVVRAGDEFEETIEGGNQSINFKPLPF
NDGEVLGAFAVVVYEDGSMNYDTMSVKEIESIKENFSKKSKDTGQ
FSKAWVVTTSEMYRKTVLRRLCKNIELDFDTIEAKQAFEDGGDFE
FNKDKKPAQESPLNPKSTVIDGEFTAVGEGAADGTE
WP_009411480.1 RecT
[Capnocytophaga sp. oral taxon 324]
(SEQ ID NO: 452)
ETQVLQKQSLANFLNKSDKFLEQNLGAKKSEFVSNLLALSDSNKE
LSQCEPADLMKCAMNATALNLPLNKNLGYAYVIPYFDGKTNRTIP
QFQMGYKGFVQLAIRSGQYKTINTCEIREGEIKRNKVTGHIDFLG
ENPSGAVIGYLAYIELLNGFQQSLFMTIEEVQAHARKYSKIYAKT
NRGLWKDEFDLMAKKTVLKLLLNRYGVLSVEMQKAIEKDQADNEG
NYIDNPQGRYIQDAEVIEQNEPTENAQPVQPVTSEEPNKVDFKDV
WP_116232802.1 RecT
[Paenibacillus sp. VMFN-D1]
(SEQ ID NO: 453)
AKALLENKLQERAAGASTPSTQGTSLKALLNSPAIKKRFDELLDK
RSAQYMTSIVNLYNSDAMLQKAEPMSVISSCIVAATLDLPVDKNL
GYAWIVPYSGKAQFQLGYKGYIQLALRTGQYKAINVIEVYEGELV
KWNPLTEALELDFEKRKSDAVIGYAGYFELINGFRKSVYWTREQI
ESHRKKFSKSDFGWKKDYDAMAKKTIIRNMLSKWGILSIEMQDAY
SKEIEAIPPLNNENEEDPPIDLTPEDYRVGDEPQDGKEQGEMNFE
WP_123849158.1 RecT
[Chitinophaga lutea]
(SEQ ID NO: 454)
SNVNAPAAPVKSKIEVLKDIMNAPSVQEQFQNALRENSGVFVASV
IDLFNSDTYLQNCEPKQVVMECLKAATLKLPINKNLGFAYVVPYK
SNGKQIPQFQIGYKGYIQLAMRTGQYRIINADKVYEGEYRTKNKL
TGEFDLSGTATSETVVGYFAHIEMLNGFAKTLYMTKEKVAAHAKK
YSKSFGKETSPWHTEFDAMALKTVLRNLLSHYGYLSVEMMGAMNA
DIESDQVGSEVSQTINDKANKQEMTFDDAEVVDDDEKEQNPI
WP_078410260.1 RecT
[Priestia abyssalis]
(SEQ ID NO: 455)
ATNQSLKNQLQSRQSAGTPAQQSNSLKALLSSPTVKKRFEEVLDK
RSAQFMTSIVNLYNSEKMLQKCEPMSVISSAMVAATLDLPVDKNL
GYAWIVPYKNTASFQLGYKGYIQLALRTSQYRFINVTPVHEGELM
KWNPLTEEIEIDFDARQSDVIIGYAAYFELLNGFRKTVYWTKNQV
EKHRKKFAKSDFGWKNDYDAMAMKTVLKAMLSKWGILSIEMQKAY
SEDEEPRELKDITEEAQEVDYIEAEVIDVPAEEKASAFDQENFHI
E
AAT90028.1 phage recombination protein
[Leifsonia xyli subsp. xyli str. CTCB07]
(SEQ ID
NO: 456)
AVKKNPTIEDYLIKYEPEFQRALGASMDAAKFAQDALTAIKQNPK
IGHSDPRSLFGALFLAAQLKLPVGGPLAQFHLTTRTVKGNLTVVP
IVGYGGYVQLIMNTGLYSRVSAFLIHAGDYFVTGANSERGEFYDF
RRADSDRGEVKGVIAYAKVKGHNESSWVYIDAETMRAKHRPKYWE
STPWADDAGEMFKKTGIRVLQKYLPKSVESLNVALAASADQAIVR
KVDGVPDLDIQHDRDTETVAVPEQPVSVPQPGDET
WP_080022455.1 RecT
[Clostridium thermobutyricum]
(SEQ ID NO: 457)
QSTGDIVFPQNYNYSNALKSAQLILAETVDRNKVPVLQSCSKPSI
CNALLDMVIQGLSPAKKQCYFVPYGGKLQLMKSYLGNIAATKRLK
GVKDVFANVIYEGDVFEYKLNLNTGLIEIEKHEQKFENISKKILG
AYAVVVRENQNNYVEVMNIEQIKNAWNQGAAKGNSQAHKNFAEEM
AKKTVINRACKRFVNTSDDSDTLIESINRTNEYKEEDIIETTKSE
VGEEIKENANTENLGLEDTEVVEAEVIENIEFEGDK
WP_081759639.1 RecT
[Clostridium jeddahense]
(SEQ ID NO: 458)
LGERTPQFISSIVSLVNADANLQRAFYDAPVTVIQSALKVATFNL
PIDPNLGYAYIVPFNNTVKNPDGSIRKRIEASFIMGYKGMNQLAL
RTGVYKTINVVDVREGELKSYNRLTEDIELDFVEDDEEREKLPII
GWVGYYRLINGTEKTIYMTRKQIETHEKKNHKGQYMGKGWREDFD
SMAMETVFRRLIGKWCLMSIDYQRANPGTLAAADALAHGQFDDED
PLPDAVPLQAEAQEVNPETGEVQS
WP_089281299.1 RecT
[Anaerovirgula multivorans]
(SEQ ID NO: 459)
DAKHLTVVHQNLNTLLKAKADALPKGFNQTRFLQNCMTVLQDTKD
IESVEPKSVARTMLKGAFLGLDFFNKECYAIVYNKKAGNSWIKTL
EFQTDYKGEIKLAKKYSINTIKDIYAKLVREGDEFEEGVKDGKQV
INFKPKPFNNNKILGAFAVAYYENGSMIYDTMSVEEIESVKKAYA
KADKEGKYSKAWIESTGEMYKKTVLRRLCKLIELDFDTIEQKQAF
DEGSGMEFKQEGKTDKPKSSLEAEFVEAEYEEVEESETSEVVEE
RDI65706.1 phage RecT family recombinase
[Nocardia pseudobrasiliensis]
(SEQ ID NO: 460)
SSIANAANASELTPASIVNRYRDDIAAVLPPKLQARIDRWLRLAI
GAVNSNADLVDRVRADQGASMMQTLMKCAALGHEPGSGLFHLVPK
GPRIEGWEDYKGVLQRIDRSGVYARVVVEVVHANDDYAYDPNLDD
RPQHKRAAADRGEPVSAYAYAVYPNGAVTAVAEATPELIAASKAK
ARGADNASSPWRAPGAPMHRKVAIRQLEKFVATSAEDMREVAVRN
AAPDVEDAPADYYQEP
WP_076170610.1 RecT
[Paenibacillus rhizosphaerae]
(SEQ ID NO: 461)
SSKLVEINSKLDSFLDAQHKAMPKGFNKTRFLQNSMSVLRDIEGL
EQCDPKSVALVMLKGAFLGLDFFNKECYPVVYAGKVEFQTDYKGE
VKLVKKYSTKPVREIYAKLVRQGDDFSEEIVAGSQTINFKPLPFN
NGEIVGAFAVVNYVDGTMQYDTMSTEEIEKIKVNFSRKSKKTNEY
SKAWVVTPGEMYKKTVLRRLCKTIDLDFDTIEQAQAFEDAADMDF
NQDSKPQQQSPLNPMVIDVEYEEVKEEQADAAEQE
WP_106833617.1 RecT
[Brevibacillus porteri]
(SEQ ID NO: 462)
ADQNKLVVIYNNLEKLLDSKREAMPTSFNKTRFLQNCMTVLQETK
DIELCNPTTVARTMLKGAFLGLDFFNKECYAVVYKGSVEFQTDYK
GEVKLAKKYSTKPVREVYAKLVREGDEFAEEINSGNQTINFRPKP
FNNEEILGAFAVVNYMDGTMAYDIMSKEEIEKIKENFSRKSKQTG
EYSKAWVVTPGEMYKKTVLRRLCKNIDLDFDTIEQRQAFEDAGDV
DFNQEVKPAQQSPLNSTVIEAEFEEVSEEQTNAAEQE
RDE19343.1 RecT
[Parageobacillus thermoglucosidasius]
(SEQ ID NO: 463)
AKQADLKNKLANKNSTNPTAYLKNLVYAPTVQQKFKEVLKEKAAH
FLTSLISLVDSSPDLQKCNPMTIIASAMKAATLELPIDKNLGYAW
IVPYKNVATFQIGYKGYIQLALRTGLYRSINVIEVYEGELRKWNR
LTEELDIDEGARKSDHVIGYAGYFELTNGFIKRVYWSKEDIERHR
KKFAKSDFGWENNYDAMAKKTVLRNMLSKWGILSIDMQRAYVNDI
DDPEQTKEVIDVEWSEIIEEANVANSPEQQEIVFEQ
WP_138600901.1 RecT
[Pseudoalteromonas]
(multispecies)
(SEQ ID NO: 464)
SLSLQEYQNLLYGKLTACKGQFDACLSENGYKLDFNTELNYVYQI
VMSGLNVEYSFPYTPVESVITSFLKAAKIGLSLCPTEQLCFLKTE
YSESSGQYVTQLGLGYKGILKLAYRSGKVKQINANVFYEKDNFQY
NGVNSKVTHTTTVLSKAMRGQLAGGYCQTELIDGSFKTTVMPPEE
ILAIEEQGKAMGNEAWLSVHVDQMREKTLIKRHWKTLCPCIYRDS
VMNDPMLFDDQDCQHSSNQQAYEEQFESAYSREAY
WP_082209600.1 RecT
[Peptostreptococcaceae bacterium VA2]
(SEQ ID NO: 465)
QPFLVQRYPHLDVVLNDQVHVLKSFFFQNHIILYLYKYIECLQIF
HKPLLKGDRGKVIGYYAVYHLEPNGYNFVFMTYDEVKNHGKKYSK
NFEGGIWEKEFDSMAKKTVIKKLLKYAPLSIEMQKAVTFDESVKG
SIDNDMLLVESIEDVEEIQLDTNI
WP_026627303.1 RecT
[Dysgonomonas capnocytophagoides]
(SEQ ID NO: 466)
STQQVQQQTKPLSLANFLNAPSTANFLKETLAEKKSEFVSNLIAL
CDADPKLAQCDPAQLMKCAMNATSLNLPLNKNLGYAYVIAYKGVP
SFQIGYKGLIQLAIRTGQYKFINATEIREGEIRHNKITGEVIFNG
EKPDAPIVGYMSYLELVNGFTASLYMTEEQIEQHALRFSQTYKND
KQYRSSTSKWSDPLARPTMCKKTVLKLLLGTYGLMTTEFAKALDS
DSDDEVSTSGHRFEEAEIVQQGEPNEEQSDEPKRMEI
WP_109523733.1 RecT
[Nocardia aurea]
(SEQ ID NO: 467)
SESISAAADAQKVTPRIVLDRHRDAFAQVLPPTINLDRWLRLAES
AINASAGLLDIFRRDRGASALKALMKCAQLGHEPGSGLFHLVPKG
QAIEGWEDYKGILQRILRSALYAKVVVAPVYANDEYAFDVNVDER
PRHKQAAGDRGEPVRAYAYAVHRDGSTSTIAEATPAMIAGAKAKG
HKTDASTSPWQNPRAPMHQKVAVRELERFVSTSAVDLRVTGDVTD
LIIEEP
GAE09585.1
[Paenibacillus sp. JCM 10914]
(SEQ ID NO: 468)
TMDYVTKIQDALDRELDAKHDALPSGFKKTRESENCRAYVKDYKD
LQKYDAEEVASVLFKGAVLGLDFLAKECHVITEGSALRFQTDYKG
EMTLVKKYSVRPILDIYAKNVREGDDFREEISGGKPLIHFNPRAF
NNSKITGSFAVALFTDGGMVYETMPAEEIESIREHYGKNPGSDTW
EKSQGEMYKRTVLRRLCKTIEIDFDAEQSLAYEAGSSFEFDREQQ
PKKRSPFNPPEVEESEVLSNDGITETQ
RRG08833.1 RecT
[Lactobacillus sp.]
(SEQ ID NO: 469)
NSLSGALNSRNQAGSPTSMIKNLMRSDSIKNRFDEVMGAKAPQFMA
SITNLVNSNQDLQHVDAMSVVASAMVAATLDLPIDPNLGYMYIVP
YRGQAQPQMGYKGYIQLALRTGQYKHINALPVYDDEVKSWNPLTE
ELEYESSGTSHDNQTPAGYVGYFQLINGFEKTTYWTYDQINSHRQ
KFSKMSSKTDPTGVWKSNFDAMALKTVLRNLISKWGIMSIEMQQA
FVKDERPQEFDHETGEIQDVQEVEAEEENVAPETQGSTDKKEE
GEA30849.1 CDIOL_17720
[Clostridium diolis]
(SEQ ID NO: 470)
ATNSSLKNQLIEKEQSTVNVQETIFKNLINSDEIKSKFTEVLKDK
AFEYINSIINLVKEIPVPNALGASDSHQSADLGSLLIECEPRSII
DACMIAASLDLSIDKNLEYVWIIPYKKKSNFQLGYKGYIQLLLRT
GEYKAINVIEVYEGQLKSWNPLTEEFDIDVSAKKSDAVIGYAGYF
EMVNGFRKYVYWSKDNMDAFRNNSFKGDPRWNNDYKAMAKRTVMR
NMLSKWGRLSAEMQRAYLEDINTDKFINGN
WP_077867213.1 RecT
[Clostridium saccharobutylicum]
(SEQ ID NO: 471)
ATNSSLKNQLIEKEQTTVNVQETMFKNLINSDDVKSKFTEVLKDK
AIQYINSIINLVNSDKDLIECEPKSIIDACMSAVSLDLSVDKNLE
YVEIIPYKKKANFQLGYRGYIQLLLRTGEYKSVNIIEVYEGQLKS
WNQLAEEFDIDFTYKKSDAVIGYAGYFEMLNGFRKSVYWSKENMD
ALRENSFKSDTRWNNDYKAMAKRAVIRNMISKWGSLSIEMEKAYC
EDLNTDKFVNGN
WP_132305216.1 RecT
[Paenibacillus] sp. BK033
(SEQ ID NO: 472)
AANTQLITIHNNLEKLIEAKKDAMPQGFNKTRFIQNCMTVLQDTY
GIEKCEPTTVARTLLKGAFLGLDFFNKECYAIPYGASMNFQTDYK
GERKLAKKYSVRKVKDIYAKLVRAGDVFEENITDGQQTIQFAPVP
FNNGDIVGAFAVVLFHDGGMLYETMSIAEMEHIKENYSKKSKDTG
KFSKAWEVSTGEMYKKTVLRRLCKNIELDFDTIEQARAFEDAADV
DFNKKTAPQQTSPLNVVEAEYEVVNDGSATEAQSE
RPI78794.1 EHM45_05245
[Desulfobacteraceae bacterium]
(SEQ ID NO: 473)
ATPNTPTTTDAGDFLKKSEKSLKNYAVRKYDFTSFLKSAMIAIND
NTTLSECLRTEAGKKSLFNAMRYAATTGLSLNPQEGKAALIGYKN
KAGEMVLNYQIMKNGLIDLALSSGKVEFVTADLVRANDEFSIKKS
ASGDDYSFSPAIRDRGEVIGFVAALKLKGSATYVKWMSTEEVAEF
RDKYSSMYKNRPDASPWTHSFNGMGIKTVMKALLRSVSISPDVDA
AVKSDDYIEAEFTVHGTTADDAVTQLQTPSKPVKAEEGQGELL
WP_051624047.1 RecT
[Clostridium akagii]
(SEQ ID NO: 474)
ATSESLKNQLVNKETRPPKDPFKALVYSAGIKKRFEDMLDKQANG
FITSLLNLKQDKLKSCDDFTVLGSALKAAALKLPIDPNLGFAWII
PFKNHGKLEAQFQIGYKGFIQMAQRSGQYKKLNVTEIYEGQLKSF
NPLTEEIVLDLDNIKSDLRKINKRYLIVMRMNLLALHLRKISKG
WP_081735325.1 RecT
[Paenibacillus gorillae]
(SEQ ID NO: 475)
LEAKHDALPSGFNAVRFVQNCKAYLPEVRNFERFNPDEIALQFLK
GAILGLDFLAKECHVITEGSAARFQTDYKGEMKLAMKHSVRPLLN
IYAKNVREGDVFRESVVEGRPVVSFDPLPFNNSKIIGSFAVAQFN
DGGMDYESMSSTEIESIRTHYGKNPGSDTWEKSQGEMYKRTALRR
LCKTIEIDFDAEQRLAFDAGSSFEFNREPRPQQQSPLNLESEVLT
DEVEQG
WP_084505057.1 RecT
[Acetobacterium dehalogenans]
(SEQ ID NO: 476)
CLRSWTRSFSNSVPLKIRFRLLYTAFLSQGSPLSSVNTTQSADKG
SPIFFYAMFKTKDGGYGFEVMSVEDVRAHAKKYSQSFSSAYSPWS
KNFEEMAKKTVLKKALKYAPLKSDFVRGIVVDETIKREISEDMYA
APSIEIEYEVDEDGVIQDEPTSNELTEAEK
AGF93134.1 RecT protein
[uncultured organism]
(SEQ ID NO: 477)
SNELQNIKPEVFGEVEDKLGSLADNNGIDLPENYSARNALKQAYL
KLQSKDEPVFDKYKDETIYNALLDTLTQGLNPGKDQVYYIGYGNH
LTAQKSYFGNIALAKRMAGVQEVSSNVILEGDEVDISIERGQQVI
ESHDRNEDSMDGQVKGAYAVISFEDERKDKYEIMTLKELKQAWAQ
GKSFGGNGKSPHHKFTKEMAKKTVINRALKPLIKASDDSGLIKEK
PKLEKLKDGQQERTEGEKIEEVDVDKEEVVEVDYDV
WP_076079849.1 RecT
[Paenibacillus] sp. FSL R7-0333
(SEQ ID NO: 478)
TVAIELQVQETLDRILDSKHDALPSDFNKKRFSENCKAYVADEKD
LHKYSPEEIAANLFKGAVLGLDFLAKECHLISGGVELKFQTDYKG
EMKLTKKYSVRPLLDVYAKNVREGDEFREEVIEGRPVIHFAPLPF
NASSIIGSFAVALFQDGGMVYESIPAGEIEEIRKNYGKSLGDAWD
KSQGEMYKRTVLRRLCKTIETDFDAEQRLIYDAGGAFEFTKQPAR
SRQQSPFNPPEESEVTQDDRVAETDQG
WP_119800346.1 RecT
[Paenibacillus] sp. 1011MAR3C5
(SEQ ID NO: 479)
ATEQIISSLEALLEAKHDALPSGFNPTRFVQNCIAYLPEIRNWDR
FNAEDLAIQFFKGAVLGLDFLAKECHIIAEGSGVRFQTDYKGEMK
LAMKHSVRPLLTIYAKNVREGDCIEEAVIEGRPVINFNPLPFNNS
SISGSFAVAQYTDGGMVYETMSAEEIEAVRTNYGKNPGSDTWDKS
KGEMYKRTVLRRLCKTIEIDFDAEQRLAFEAGSEFDFSKQPRPQQ
RSPFEEKEVGPDEVEQG
WP_025706233.1 RecT
[Paenibacillus graminis]
(SEQ ID NO: 480)
TVAIETQVQETLDRILDSKHDALPSDFKKKRFSENCKVYVAEEKD
LHKYTIDDIVANLFKGAVLGLDFLAKECHLITGGVDLKFQTDYKG
EMKLTKKYSVRPMLDVYAKNVREGDIFREQIIEGRPAIHFDPLPF
NASKIIGSFAIALFQDGGMVYESIPAGEIEEIRKNYGKSLGDAWE
KSQGEMYKRTVLRRLCKTIETDFDAEQRLIYEMGGAFEFTKQPTR
SRQQSPFNPPEESEVIQNDRAAETDQG
OIO76374.1 AUJ88_06865
[Gallionellaceae bacterium CG1_02_56_997]
(SEQ ID NO: 481)
GRKEVERIRDGSRGYQAAKKYKKESTWDTDFVAMGLKTAIRRICK
FLPKSPELATALAMDEQAGRQNLNLDDVINGSYTPVVDKDTGEIV
DVADGGKTGNSNAATSTKLEKLEKIVAALRDANSVEALDEIYIRA
EGDLDDANLEIAMREYRKCKDAISNSLI
WP_131535536.1 RecT
[Pedobacter nototheniae]
(SEQ ID NO: 482)
STEQSQQQTAARVPAKFQEGTVDSILKRVSDFQNTGELVLPANYI
PENAVRAAWLMLMETTDRNDKPAIEVCTKESIANAFLEMVTKGLS
VVKKQCYFVVYGNKLSLEDSYIGKIAIAKREAGVKEVNAVTIYEG
DIFKYENDIETGRKRILEHKQELKNINPDKIVGA
WP_028113352.1 RecT
[Ferrimonas kyonanensis]
(SEQ ID NO: 483)
NQMINEPDFVTALKDSRETYIDLTQNGGFNLNYGLEAGWAHQQIE
ASRYQNLDLTCSEPGSIMQAFCEAARLGLSFDPRKKHIYLMGQKD
VQSGRTITILYVGYKGMIALACRTGFMIGGHADLVFEEDTFTYRS
GTQLPVHEHDGRPNHERGRLKCGYVVAHQPGGMVKTLLVPKEVLL
EAASNGLNAGGSNNTWCGPYMEMMYQKTCWRYAFNAWYSELEAVG
MTQAQLESATTAVSYQ
WP_100916003.1 RecT
[Pseudoalteromonas spongiae]
(SEQ ID NO: 484)
NKFQHLQTELSSQLLSTKERFNELNNKNNLKVNFEEEYNFFYHLV
TSSFYNINGIATCTFSSLKEAFLNIAKYGLSINPKLNLCYIRTEQ
SCAQANVNIAVYDFGYKGLLKLITRTGKVKIVTADVFYENDNFEF
RGTREPVKHSTKTLSAAARGAMAGGYCSSELVAGGVVTTIMTPEE
LREIESICQSTGNEAWNSVFIDELRRKTLIKRHWKTLMQVIEEQN
LSVPIEETYQCDFANGGY
WP_125711747.1 RecT
[Companilactobacillus kedongensis]
(SEQ ID NO: 485)
MKDLARIPVKELVRSDTIKSKFNDVLGKRAPQFISSIVNIVNSNQ
DLKNVDQTSVISSALVAASLDLPINQSFGYMYLVPYSGKAQPQMG
YKGYIQLAQRSGQYKRLNAISVSKEKVPDKMVIFIPDYRMEEAET
QIDMYQDHIEDVKAGRVEPTRCGKCDYCKSTAKLGKIVSMDDLID
WP_002845682.1 RecT
[Peptostreptococcus anaerobius]
(SEQ ID NO: 486)
SNQVTESKKGYVAEKNITDSALNAINKYMNDGVLHLPKTYSVENA
MKSAYLTLSQAKDKNGKSVLESCTKESIYQSLLDMAVQGLTPAKN
QCYFIPYGSKLTMSRSYLGTIAVTKSAVPEVKDVKGYAIYDKDVF
ETEFDYNTGCIKIKKFERNFDSIDTNSIKGAFALIIGEHGVLHTE
VMNMAQIRNAWSMGATNGKSKAHNQFTDQMAIRTVINRACKFYIN
TSDDTSVLFADSYANSDEDTSSEREVEIVDENVREKK
WP_115407185.1 RecT
[Shewanella morhuae]
(SEQ ID NO: 487)
QTAQVKLSVPHQQVYQDNFNYLSSQVVGHLVDLNEEIGYLNQIVF
NSLSTASPLDVAAPWSVYGLLLNVCRLGLSLNPEKKLAYVMPSWS
ETGEIIMKLYPGYRGEIAIASNFNVIKNANAVLVYENDHFRIQAA
TGEIEHFVTSLSIDPRVRGACSGGYCRSVLMDNTIQISYLSIEEM
NAIAQNQIEANMGNTPWNSIWRTEMNRVALYRRAAKDWRQLIKAT
PEIQSALSDTEY
WP_081955873.1 RecT
[Helicobacter trogontum]
(SEQ ID NO: 488)
SNITTIQRKNEALALLENKEIQERLCALCGNEASKDKFKASLLNI
ALDSNLSACSMQSIVKASLDIAGLKLSLNKNLGKAYIVPRKVKIG
NDYITEARIDIGYKGWLELAKRSKLSVKAHSVFDCDDFVYSVDGV
DEYMKLTPNFELRQEHDSAWVKEHLKGIVVGIKDLKSGDSEVKFV
SKGTLLKIMQKNDSVKNGKYSAYTDWLHEMLLAKAIKSCLSKTAM
SEDTFYLIISNNKLFI
WP_064664300.1 RecT
[Pseudoalteromonas] sp. MQS005
(SEQ ID NO: 489)
SISQQDYENLLYSKLYECESQYQAYLAEHNEKLNFNAELNYMYKA
VMSGVGIEGGFPYTPLESIVESFLKAAKLGLSLDPSEQFCFLRSQ
YDHSTGLYHTELGLGYKGVLHLAYRSGKVKQIVSNVFYNKDNFQF
NGPNSKVTHTMTVLSTSARGNLAGGYCQTELVDGSFIVTVMPPEE
ILAIEEQGKSVGNPAWLSAHVNQMREKTLILRHWKTLYPAIYSSS
LLDSAQIFDDECEEFPFSSPSQGFSESQTIGSY
WP_069455496.1 RecT
[Shewanella xiamenensis]
(SEQ ID NO: 490)
QTAQVKLSVPHQQVFQDNFNYLSSQIVGHQVDLNEEIGYLNQIVF
NSLATTSPLDVAASWSVYRLLLNVCRLGLSLDPEKKLAYVIPSLS
ETGEKIMKLYPGYRGEIAIASNANVLKNANAVLVYENDHFRIQAA
TGEIEHFVTSLSIDPRVRGACSGGYCRSVLMDGSVLMSYLSIEEM
DSIAQHQIEANMGNTPWNSIWRTEMNRVALYRRAAKDWRQLIKAT
PEMQSSLLDTEF
RTL04618.1 EKK58_09925
[Candidatus Dependentiae bacterium]
(SEQ ID NO: 491)
CHVLNFQTDYKGEIKLAHKYSVRKIIDIYAKVVRDGDVLEIRVEN
GSQIVNFNPKVENDGKIIGAFAVVKFVDGSLLYETMSKSEIDHTR
VTFSKMPNGMAWKDSEGEMCRKTVLRRICKLIDLHFDSVEQEQAW
NDGSDADLTKNEPVKPEIQNPFPTKAVEAVIVTEEEKLRKQLKDK
DPTLQDWQIDALVREHKEANQ

### Example 18

Exemplified by CRISPR-Cas9 systems, gene editing has become a powerful tool for probing the mechanisms of human health and diseases. Cas9 editing can cause DNA damage at on- and off-target sites and rely on the endogenous DNA repair mechanisms that are error-prone. These features often lead to unwanted mutations and safety concerns, which can be exacerbated when we alter long sequences. Building on prior studies that mammalian genome DNA becomes transiently accessible upon dCas9 DNA-unwinding and R-loop formation, we hypothesized that microbial single-strand annealing proteins (SSAPS) could stimulate DNA strand exchange for gene-editing when coupled to dCas9-guideRNA complex. Thus, we developed a cleavage-free gene-editing tool using the catalytically-dead dCas9 for knock-in long sequences. Our data demonstrated that this dCas9-based editor had very low editing errors at target loci, minimal detectable off-target effect, and higher overall accuracy than Cas9 editors. Meanwhile, dCas9-SSAP editor had comparable efficiencies as Cas9 editors, with robust performances across human cell lines and stem cells. This dCas9-SSAP editor was effective for inserting sequences of variable lengths, up to kilobase scale. In experiments where we chemically inhibited DNA repair enzymes, dCas9-SSAP editing demonstrated notable independence from endogenous mammalian repair pathways. For convenient viral delivery of the dCas9-SSAP editor for challenging cell types, we performed truncation and aptamer engineering to minimize its size to fit into a single AAV vector for future applications. Overall, this tool opens opportunities towards safer genome engineering in mammalian cells.

Since the initial demonstration of CRISPR-Cas9 gene-editing, significant efforts have improved and expanded gene-editing technologies for studying genome function, modeling biological processes, and gene therapies. New generations of gene-editing tools, such as base editing and prime editing, substantially improved the efficiency and fidelity of gene editing and are powerful for altering relatively short sequences. Most gene-editing tools work by cleaving genome DNA to induce single-strand nicks (SSNs) or double-stranded breaks (DSBs) that facilitate targeted editing. These DNA modifications are often repaired by error-prone endogenous pathways such as non-homologous end-joining (NHEJ). This process often leads to unwanted mutations and off-target effects, which could result in toxicity and raise safety concerns. Such editing errors and off-target effects would become increasingly and sometimes prohibitively severe when engineering long genomic sequences (>=100 bp). These unwanted effects limit the application of gene-editing to engineering large-scale genomic knock-in or in vivo gene-editing.

Available CRISPR-based methods for long-sequence editing, such as homology-directed repair (HDR) or microhomology-mediated end-joining (MMEJ), rely on Cas9 cutting and often trigger random indel formation within the genome. Many recent efforts have enhanced precision long-sequence editing, such as chemical enhancers, fusion of enhancement domains, and modified donor DNAs. Nicking-based HDR has been shown to reduce editing errors but could lead to lower efficiency. Thus, there remains a need for efficient, safer CRISPR editing tools for long-sequence alterations.

Bacteriophages evolved enzymes that take advantage of accessible replicating genome DNA to perform precise recombination. We reasoned that the key enzyme for microbial recombination, the single-strand annealing protein (SSAP), could be useful for gene editing in mammalian cells, would not rely on DNA cleavage, and not trigger the error-prone pathways involved in Cas9 editing. Motivated by this hypothesis and our prior work showing its ability to stimulate genomic recombination, we developed a gene-editing tool using the deactivated Cas9 (dCas9, or catalytically dead Cas9) and microbial SSAPs. This dCas9 editor uses the SSAP for knock-in editing when supplied with a donor DNA, without the need for genomic DNA cleavage. We termed it dCas9-SSAP editor (dCas9-SSAP).

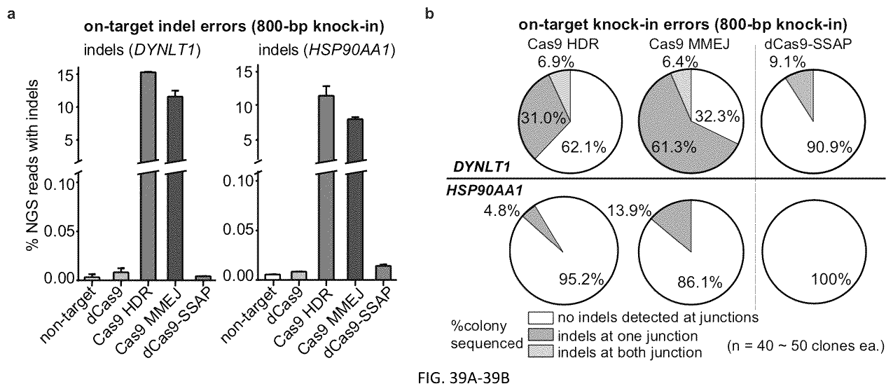

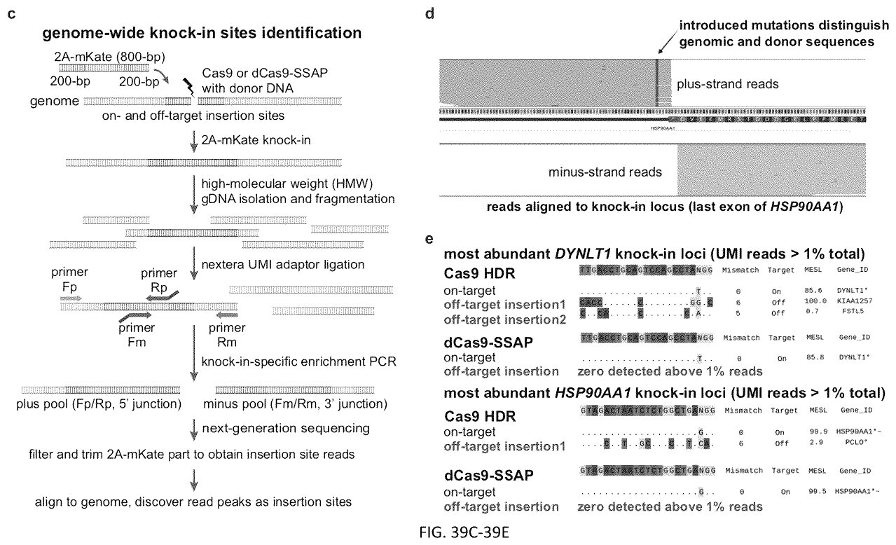

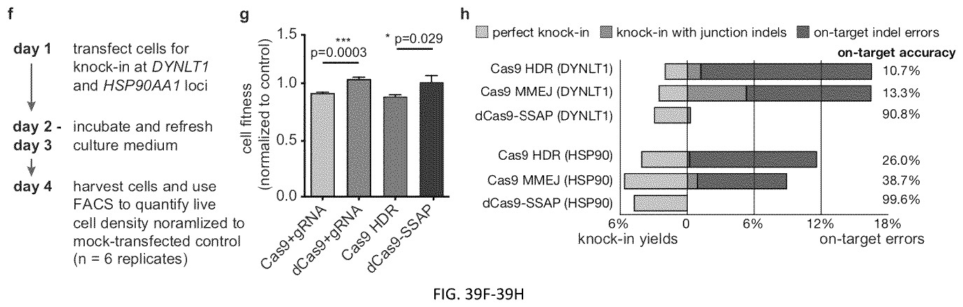

To optimize dCas9-SSAP, we performed a metagenomic search of SSAPs focusing on RecT homologs, and identified EcRecT as the most efficient one for human genome knock-in. For validation, we conducted a series of genome engineering and chemical perturbation experiments. Our data showed that dCas9-SSAP had comparable knock-in efficiencies to wild-type Cas9 references, with efficiencies significantly higher than Cas9 nickase editors. dCas9-SSAP achieved up to 12% knock-in efficiency without selection, across multiple genomic targets and cell lines, for kilobase-scale sequence editing. More importantly, our data showed that this new tool generates nearly zero on- and off-target errors. In an assay for 1 kb-sequence knock-in, dCas9-SSAP had less than 0.3% editing errors across all cells, while Cas9 editors had similar yields but an additional 10%-16% incorrectly-edited cells. Across loci tested, dCas9-SSAP had 90%-99.6% editing accuracies, while Cas9 editors' accuracy ranges from 10% to 38% (FIG. 39F).

Further, we probed the mechanism of dCas9-SSAP editing via inhibiting several DNA repair enzymes and performing cell cycle synchronization. In these experiments, dCas9-SSAP demonstrated less dependence on the endogenous DNA repair pathways, as opposed to Cas9 editing. Results of our cell cycle assays supported our hypothetical mechanism of dCas9 editor; they are consistent with the known biophysical, biochemical properties of dCas9.

Finally, to help with delivery of dCas9-SSAP for future applications, we optimize its molecular design using structural-guided truncation, and obtain a minimized dSaCas9-mSSAP, achieving over 50% reduction in size and retaining similar levels of efficiency. This minimal dCas9 editor would allow convenient delivery using viral vectors such as adeno-associated virus (AAV), potentially useful for hard-to-transfect cell types or in vivo applications. Overall, the dCas9-SSAP editor is capable of efficient, accurate knock-in genome engineering. With space for further improvement, it has potential research and therapeutic values as a cleavage-free gene-editing tool for mammalian cells.

Using Phage SSAPs for dCas9 Knock-In Gene Editing

Most CRISPR-based editors capable of long-sequence knock-in require SSNs or DSBs, which can trigger the competing, error-prone NHEJ pathways, resulting in variable efficiency and accuracy. In contrast, bacteriophages evolved DNA-modifying enzymes to integrate themselves into the genomes of host bacteria via sequence homology, e.g., Lambda Red. Such precise phage integration relies on a major homology-directed step: recombination between genomic and donor DNA is stimulated by the SSAPs, e.g., Lambda Bet or its functional homolog, RecT. From prior studies, we reasoned that phage SSAPs may not rely on DNA cleavage thanks to its unusual ATP-independent activity, in contrast to the ATP-dependent RAD51 protein in human cells. Phage SSAPs' high affinity for single- and double-stranded DNAs may allow attachment to donor templates when multiple SSAPs are recruited to genomic targets via RNA-guided dCas9. It could then promote genomic-donor DNA exchange without cleavage, as target DNA strands become transiently accessible during dCas9-mediated DNA-unwinding and R-loop formation.

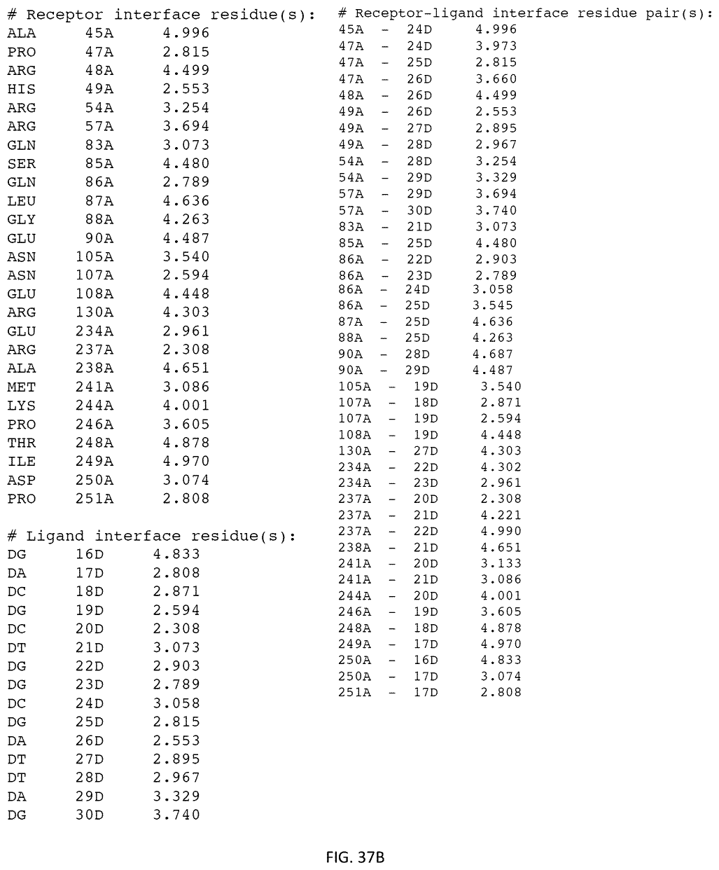

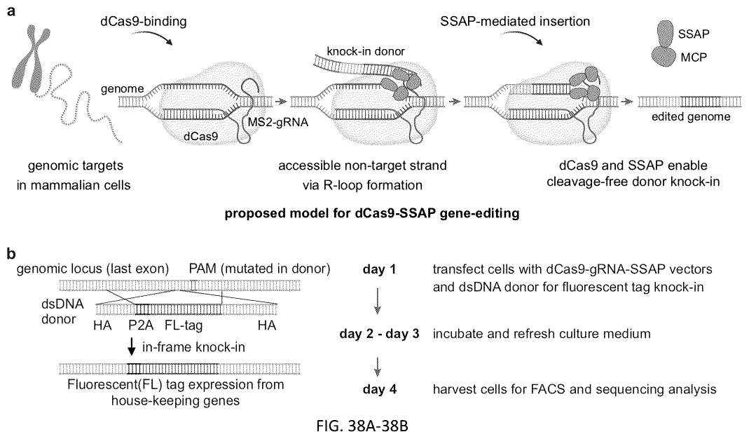

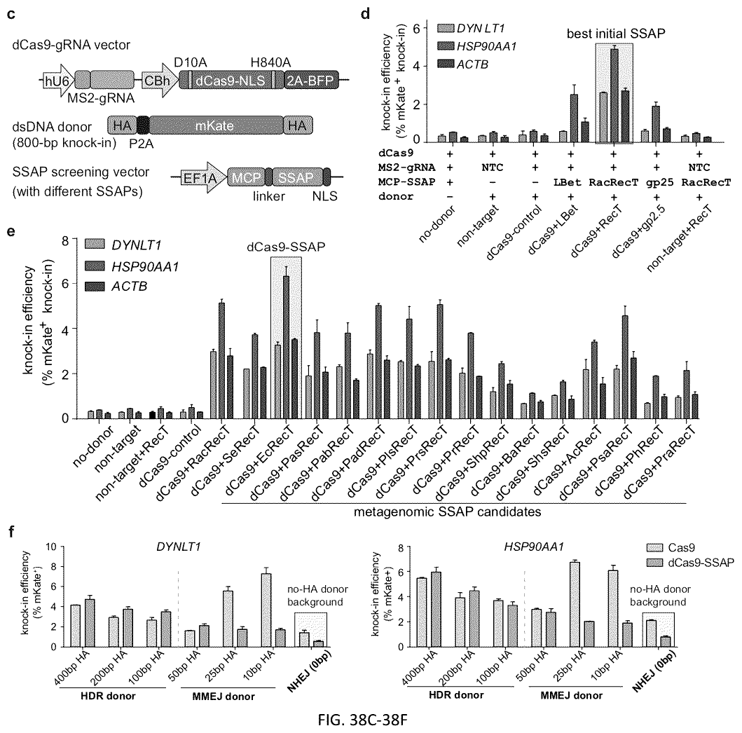

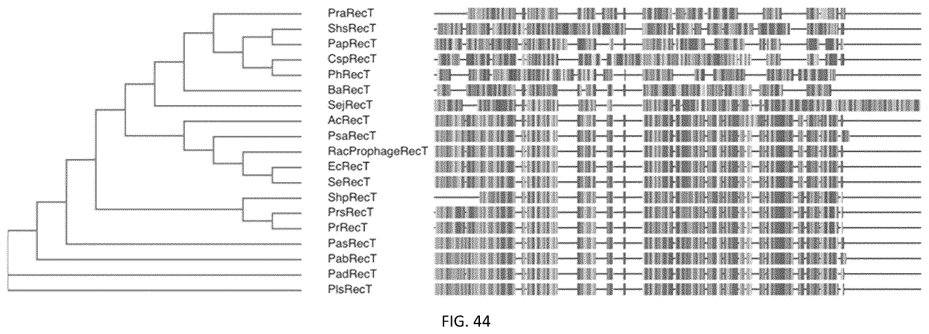

Based on this hypothesis, we designed a system to recruit SSAPs to catalytically-dead Cas9 (dCas9) (FIG. 38A). The dCas9 protein cannot cut DNA but retains the ability to unwind target sites and form R-loop, rendering the non-target strand putatively accessible for SSAP-stimulated homologous recombination. To test this, we engineered and evaluated three major types of microbial SSAPs: lambda Bet protein (lambda bet); E. coli Rac prophage RecT (Rac RecT), and phage T7 gp2.5 (T7 gp2.5). We recruited these SSAPs to the deactivated version of S. pyogenes Cas9 (dSpCas9, simplified as dCas9 hereafter) via an RNA aptamer MS2 stem-loop (FIGS. 38A, 38C). This MS2-aptamer was inserted into sgRNA scaffold, and the candidate SSAPs are fused to an N-term MS2 coat protein (MCP) that binds specifically to the MS2 aptamer, thus allowing multiple SSAPs to form a complex with dCas9-guideRNA. To measure their gene-editing activity in human cells, we generated knock-in donors with an 800-bp transgene encoding fluorescent protein (FP) cassette flanked by homology-arms (HA), which allow in-frame insertion of the FP into housekeeping genes, e.g., DYNLT1, HSP90AA1, ACTB (FIG. 38B, left). Upon precise knock-in, we measured the percentage of FP-expressing cells to quantify the gene-editing efficiency (FIGS. 38B-38D). Our initial test identified that RecT has higher knock-in editing activities relative to other SSAPs in human cells, whereas no editing above background was observed with dCas9-only or non-target controls (FIGS. 38C, 38D). We validated this knock-in editing using gel electrophoresis and sequencing (FIG. 44). This provided evidence that coupling SSAP to dCas9 via RNA aptamer enables knock-in gene-editing.

Development of dCas9-SSAP as a Mammalian Gene-Editing Tool

We conducted metagenomic mining to identify the best SSAP for mammalian gene-editing. We focused on RecT homologs and sought to maximize evolutionary diversity via a phylogenetic analysis. We systematically searched the NCBI non-redundant sequence database for RecT homologs, and identified 2,071 initial candidates. Then we built phylogenetic trees, filtered out proteins with high sequence homology, and subsampled the evolutionary branches, obtaining 16 highly diverse SSAP candidates (FIG. 44).

We examined the SSAP candidates by knock-in screening and evaluating their editing efficiencies across three genomic loci: HSP90AA1, DYNLT1, and ACTB (FIG. 38E). Among all candidates, EcRecT demonstrates the highest efficiency for dCas9 editing—it achieves genomic knock-in of kilobase cassette with up to ˜6% efficiency in human cells. This was significantly higher than dCas9 controls without SSAP, which were comparable to the no-donor controls, suggesting that dCas9 alone cannot perform genomic knock-in (FIG. 38E). To measure possible background insertion of donor DNA, we included non-target controls using guide RNAs that do not recognize the genomic targets and observed comparable activity to the no-donor negative control (FIG. 38E). We also tested SSAP with a non-target control, confirming that expressing SSAP alone is not sufficient for knock-in (FIG. 38E). Lastly, we tested the new editor with different donor DNA designs (FIG. 38F). Our results suggested that SSAP-mediated editing is more efficient when using HDR than MMEJ donors, and longer homology arms in general make the editing efficiency higher (FIG. 38F). This is consistent with prior reports that MMEJ rely on DNA breaks which are missing in dCas9 editing. Taken together, the proposed dCas9 editor enabled efficient knock-in editing in human cells, with EcRecT as the top SSAP. In what follows, we focus on this top design, referred to as dCas9-SSAP.

Characterizing the Accuracy of dCas9-SSAP Gene-Editing

The motivation for developing dCas9-SSAP is to perform potentially safer, cleavage-free dCas9 editing with the help of SSAP. Thus, we experimentally evaluated the accuracy of dCas9-SSAP for knock-in editing where the target sequence is ˜1 kb in length. We measured the on-target error, off-target insertion, cell fitness effect, and editing yields of dCas9-SSAP, in comparison with Cas9 references.

On-target error analysis. There are two types of on-target errors: (1) on-target indel formation, whose occurrence means that knock-in is unsuccessful; (2) knock-in errors, which means that knock-in happens but is imperfect, and that junction indels occur.

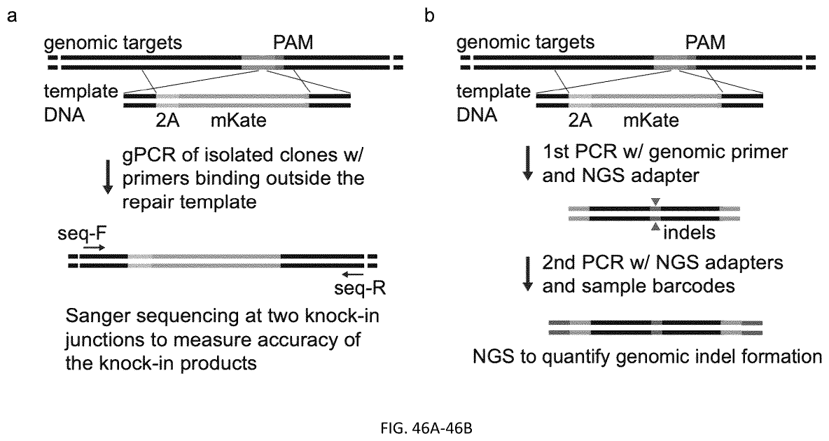

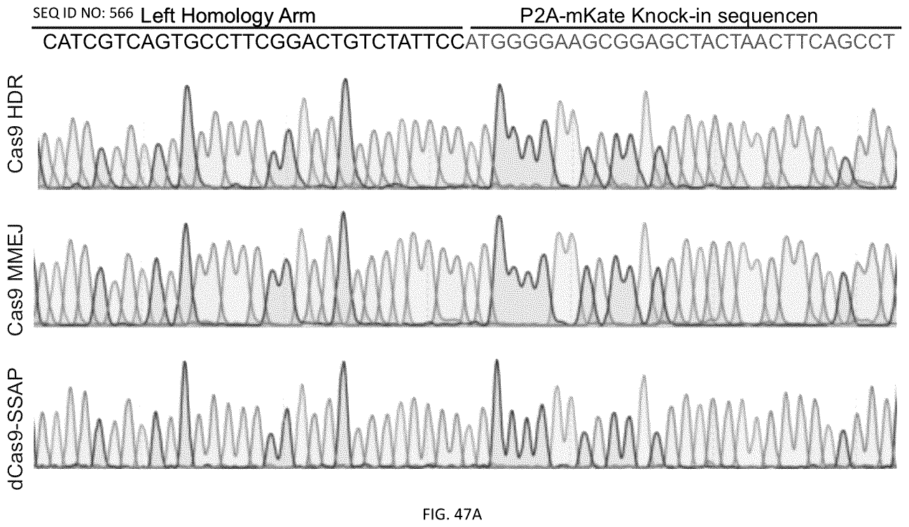

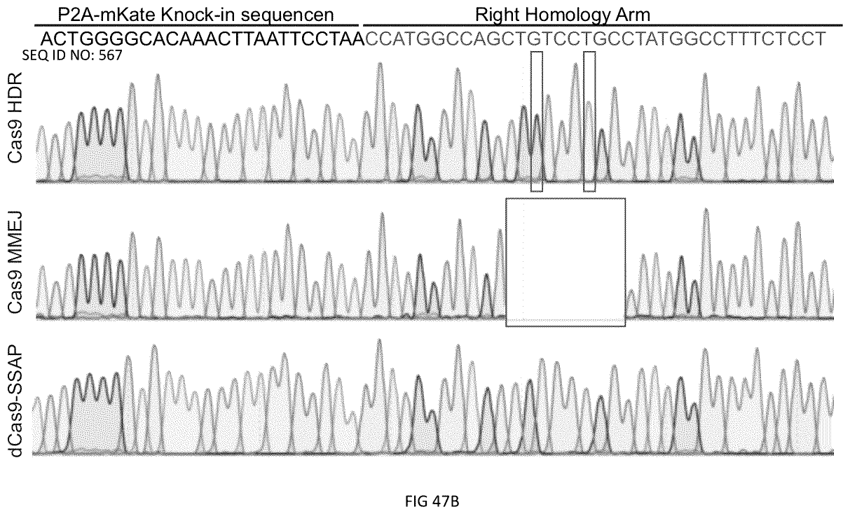

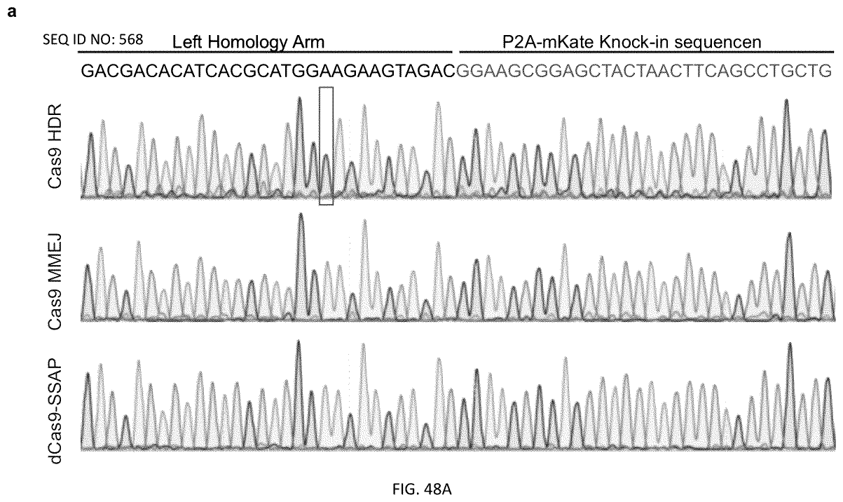

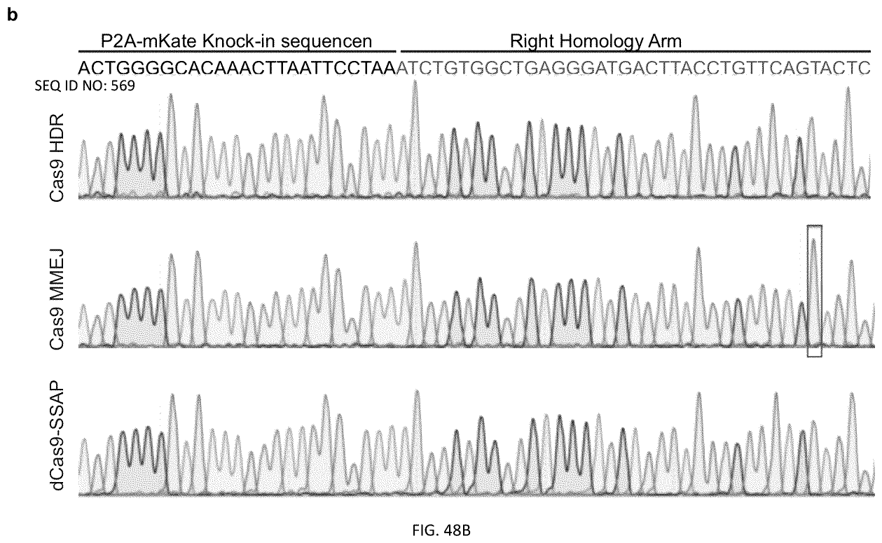

To evaluate type (1), we used deep sequencing to measure the on-target indel formation of dCas9 editor. We used the nested PCR design with an initial primer binding outside the donor DNA to avoid template contamination (FIG. 39A, FIG. 46). Deep sequencing of on-target sites showed that the dCas9 editor's level of on-target error is as low as that of negative controls, in contrast to high levels of indel formation observed for Cas9 editor (FIG. 39A).

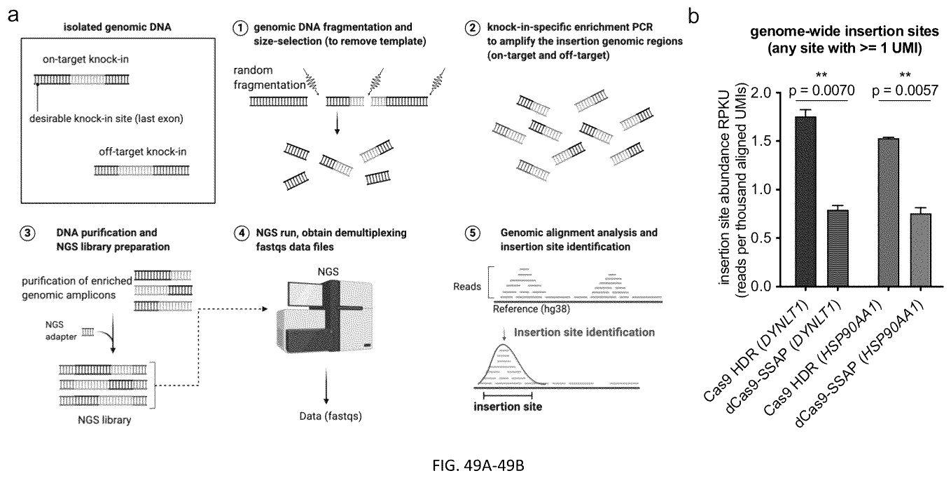

To evaluate type (2), we benchmarked the knock-in errors of dCas9-SSAP and measured junction indels. We clonally isolated edited cells, and then amplified the knock-in genomic loci using a similar 2-step nested PCR design to avoid contamination (FIG. 39B, FIG. 46), we assessed the edited genomic alleles via Sanger sequencing. The long-read Sanger sequencing allowed us to thoroughly examine the entire knock-in junctions. Our results indicated that, while MMEJ donors are more efficient than HDR donors when using Cas9, they also led to significantly higher percentage of editing errors (FIG. 39B). More importantly, dCas9-SSAP outperformed Cas9-HDR and Cas9-MMEJ in terms of the percentage of clones with no knock-in errors (FIG. 39B, FIGS. 47-48). At one locus, dCas9-SSAP achieved 100% knock-in success (within limit of assay sensitivity, see Methods).

Off-target error analysis. We evaluated the off-target knock-in error of dCas9-SSAP editing via a genome-wide transgene insertion assay (FIGS. 39C-39E, FIG. 49). Briefly, we isolated high-molecular weight genomic DNA, followed by fragmentation and UMI-adapter ligation, and then used transgene-specific primers for unbiased identification of insertion sites within the genome (FIG. 39C). Through a previously-validated analysis pipeline modified from Cas9 genome-wide off-target work (Methods), we were able to identify enriched peaks of reads that represent high-abundance transgene insertion sites (FIG. 39D). For this analysis, we also performed down-sampling to ensure all groups have the same sequencing depth/coverage. Considering insertion sites with >1% of total aligned reads, our results confirmed that dCas9-SSAP had no detected off-target insertion site, while Cas9 references led to a significant number of off-target error sites (FIG. 39E). Notably, in all dCas9-SSAP samples, there were significantly less off-target sites when we consider all sites with at least one UMI aligned, in contrast to Cas9 editor (FIG. 49). This result suggests that dCas9-SSAP could help to address the off-target issues that are prominent for long-sequence knock-in.

Cell fitness effect and editing yield analysis. We also compared the fitness of cells that went through Cas9/dCas9-based editing. We experimented with two target sites and our data suggests that dCas9 editing in general leads to higher cell fitness than Cas9 editing (FIGS. 39F, 39G, defined by the normalized percentage of live cells after editing).

For the full picture, we summarized editing yields for dCas9-SSAP with comparison to Cas9 references. We tabulated the percentage of accurate knock-ins, percentage of knock-ins with errors, and the percentage of on-target indels without knock-ins, where the sum of latter two is the total on-target errors (FIG. 39H). We also measured the overall accuracy rate of editing, which is defined by the ratio between successful knock-in cells and total edited cells (FIG. 39H). In this analysis, we observed that Cas9 editors suffered from frequent errors for long-sequence editing, where the percentage of erroneous edits are significantly higher than the yields, and their accuracy rate ranges from 10% to 38%. While dCas9-SSAP had similar levels of knock-in yields with the best Cas9 references, it had minimal error and achieved 90%-99% accuracy rate across genomic loci.

Benchmarking the Efficiency of dCas9-SSAP Editing with Cas9 Editing

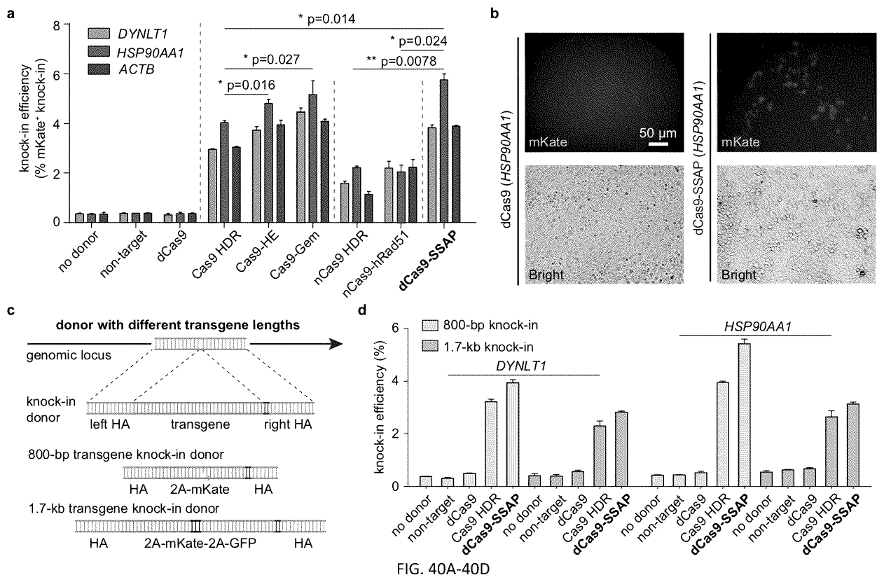

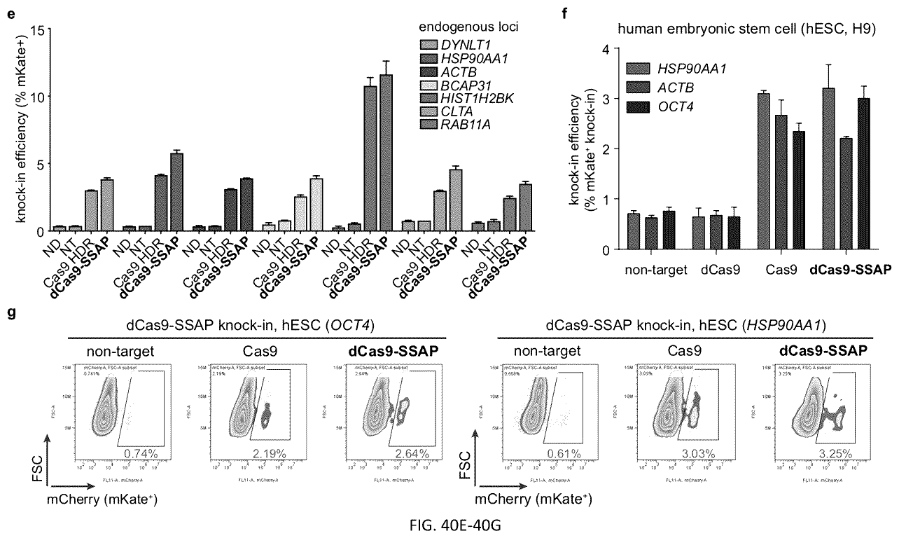

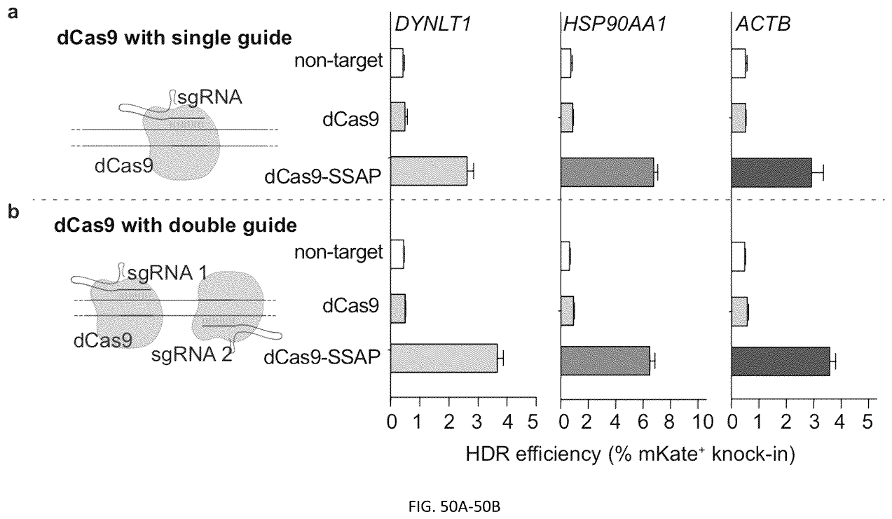

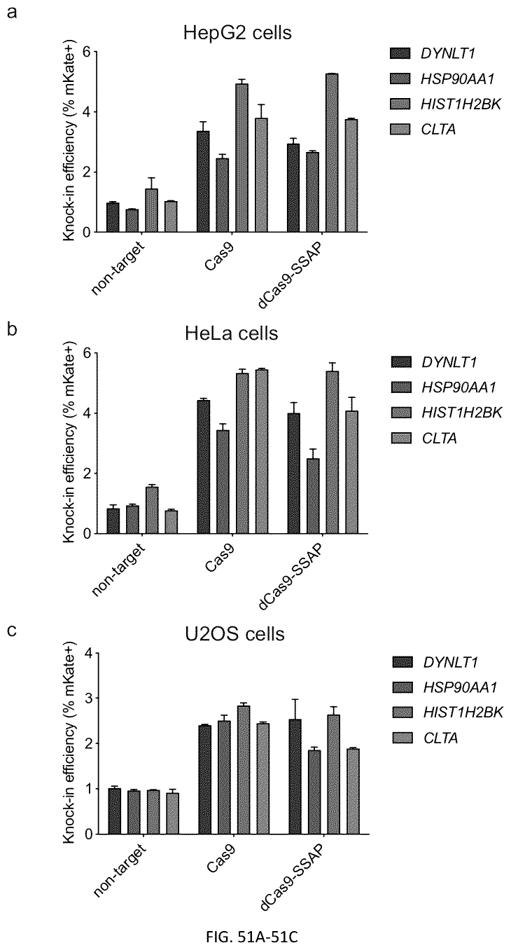

Having established that dCas9-SSAP has higher accuracy for knock-in editing, we further validated its efficiencies and usages. We benchmarked its editing efficiency across different cell lines. For benchmarks, we experimented with both wild-type and nicking-based Cas9 (nCas9) editors, including three HDR-enhancing tools. We examined their 1-kb knock-in activities across the three genome targets in human HEK293T cells. Results from this comparison demonstrated that dCas9-SSAP achieved higher efficiencies than the Cas9, nCas9, and nCas9-hRAD51 nickase editors, with comparable efficiencies as Cas9-HE and Cas9-GEM, two published HDR-enhancing editors (FIG. 40A). Additionally, our data showed that a single-guide dCas9-SSAP editor was sufficient for effective knock-in, with minor improvement when using two guide RNAs (FIG. 50). Thus, we concluded that dCas9-SSAP had similar levels of efficiency as the Cas9-based editors.

Next, we evaluated the editing efficiencies of dCas9-SSAP with different donor DNA designs (FIG. 40C). Our results indicated that SSAP-mediated editing is more efficient when using HDR than MMEJ donors and longer HAs generally result in a higher editing efficiency. We evaluated the editing efficiency of dCAS9-SSAP when the sequence for knock-in has variable length, up to 2-kb for dual-FP knock-in (FIG. 40D). Our data showed that dCas9-SSAP had consistent performances, with comparable and often higher efficiencies than Cas9 references across the transgene lengths tested (FIG. 40D).

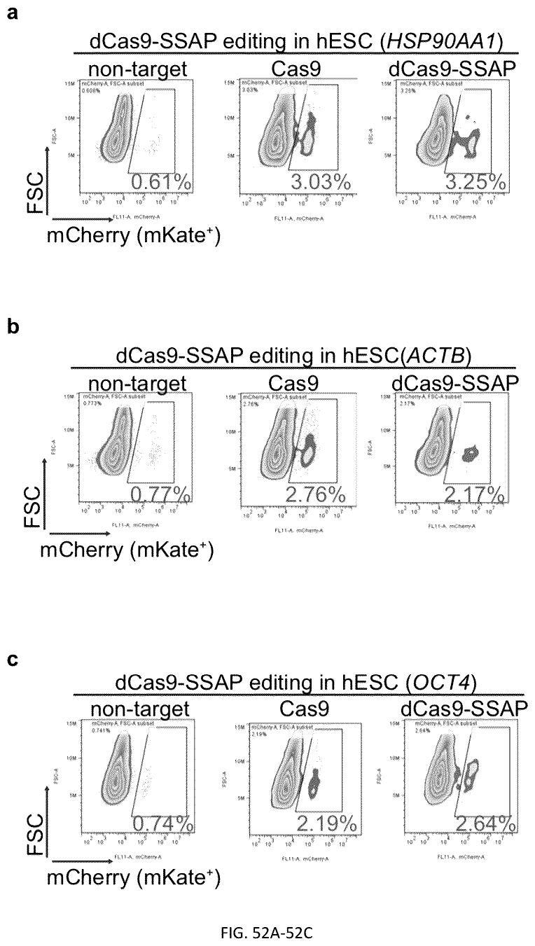

Lastly, we tested if dCas9-SSAP editor has robust activities across genomic targets, and if it is applicable in more challenging cases beyond one model cell line. We selected four additional endogenous loci from house-keeping genes (BCAP31, HISTIH2BK, CLTA, RAB11A) in addition to the three previously tested ones (DYNLT1, HSP90AA1, ACTB) (FIG. 40E). Across all genomic sites, dCas9-SSAP editor demonstrated efficiencies up to 12% without selection, comparable and often slightly higher than Cas9 references using the same donors (FIG. 40E). Further, we applied dCas9-SSAP to three cell lines with distinctive tissue origins (cervix-derived HeLa cells, liver-derived HepG2 cells, and bone-derived U-20S cells). We observed consistent knock-in efficiencies comparable to Cas9 references in all three lines (FIG. 51). Finally, we used dCas9-SSAP editor in human embryonic stem cells (hESCs) to engineer sequences in a more therapeutically relevant setting. We observed robust knock-in editing activity across all three genomic sites tested (FIGS. 40F, 40G). Of note, dCas9-SSAP editing used short ˜200-bp HAs and achieved up to ˜3% efficiency for kb-scale editing without selection, comparable and often higher than the Cas9 references in human stem cells (FIG. 40G, FIG. 52).

Chemical Perturbations Suggest dCas9-SSAP Gene-Editing has Less Dependence on Endogenous DNA Repair Pathways

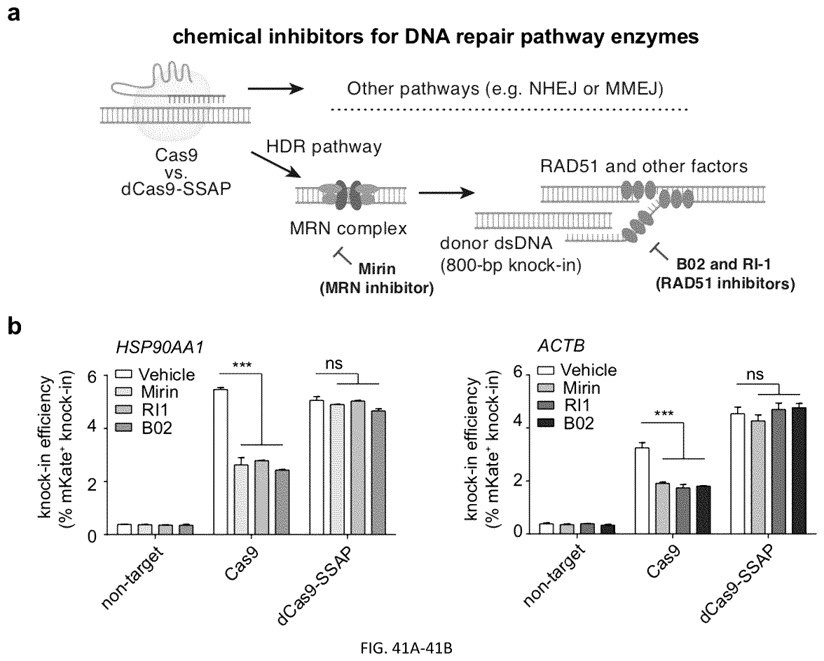

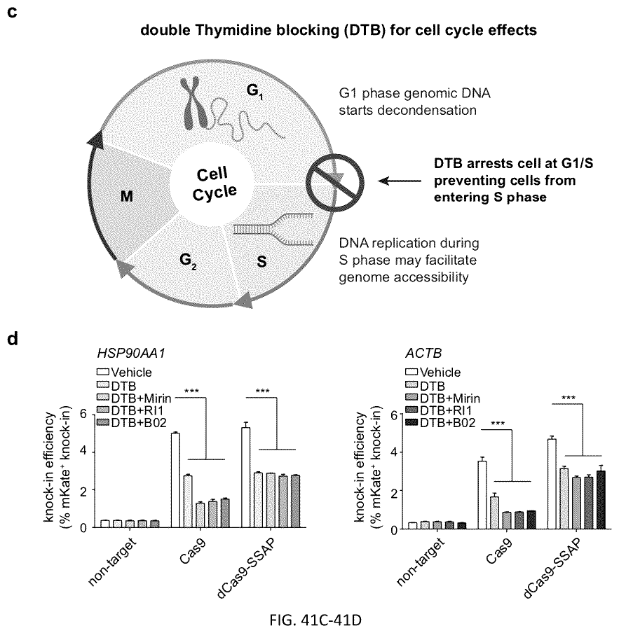

Recall our model that dCas9-SSAP performs gene editing without DNA cleavage or dependence on an endogenous repair pathway. To better understand the nature of dCas9-SSAP editing, we used three orthogonal chemical perturbations to probe its mechanism (FIG. 41).

First, we investigate if the dCas9-SSAP editing depends on the DSB repair pathway as Cas9 editing docs (FIG. 41A). In Cas9-mediated knock-in, the recognition of DSBs by the Mre11-Rad50-Nbs1 (MRN) complex is a necessary step for downstream HDR repair. We leveraged Mirin, a potent chemical inhibitor of DSB repair, which has been shown to prevent MRN complex formation, ATM activation, and Mre11 exonuclease activity. We treated cells with Mirin and tested the editing efficiencies of dCas9-SSAP and Cas9 references on these cells. Across all genomic targets, we observe that the dCas9-SSAP efficiencies were nearly unaffected by the Mirin treatment and essentially the same as vehicle-treated groups (FIG. 41B, Mirin). Meanwhile, Cas9 references demonstrated substantially reduced editing efficiencies under the Mirin treatment, which suggests Cas9 editing depends on the DSB repair (FIG. 41B, Mirin).

Second, we investigated the dependence of dCas9-SSAP on the HDR pathways. We used two small-molecule inhibitors of the HDR enzyme RAD51, RI-1 and B02, to block this rate-limiting step. Our data showed that blocking RAD51 activity via these two inhibitors significantly reduced Cas9 editing efficiencies at all genomic targets, but it did not have a significant effect on dCas9-SSAP editing (FIG. 41B, RI1 and B02). These two repair-modulating experiments generated consistent results: dCas9-SSAP showed significantly less dependence on the endogenous DNA repair mechanisms than Cas9 references. They suggest that dCas9-SSAP acts through the activity of SSAP when recruited by the dCas9-guideRNA complex and differs from Cas9 editing.

Third, we investigated how cell cycling affects the dCas9-SSAP editor. Cell cycling has been shown to facilitate the accessibility of mammalian genomes. More specifically, the genome replication (during S phase) may provide a favorable environment for the dCas9 to unwind DNAs and allow SSAP-mediated recombination (FIG. 41C). To test this potential effect, we synchronized cells at the G1/S boundary using the double Thymidine blockage (DTB). DTB treatment indeed reduced dCas9-SSAP editing efficiencies (FIG. 41D). Nonetheless, when we combined Mirin, RI-1, or B02 with DTB treatment, dCas9-SSAP maintained higher editing efficiencies than Cas9 references across genomic loci tested (FIG. 41D). This further supported that the dCas9-SSAP editor had less dependence on endogenous repair pathways.

Taken together, our data supported the hypothetical mechanism of dCas9-SSAP editing: RNA-guided dCas9 binds to genomic targets and makes them accessible to the SSAP, so SSAP would promote homology-directed recombination without generating any DNA break (FIG. 38A). Deeper understanding into this process will require further investigation, e.g., biophysical analysis of the dCas9-SSAP complex as it performs gene-editing or additional assays to perturb mammalian genome accessibility.

Minimization of dCas9-SSAP Gene-Editing Tool for Convenient Delivery

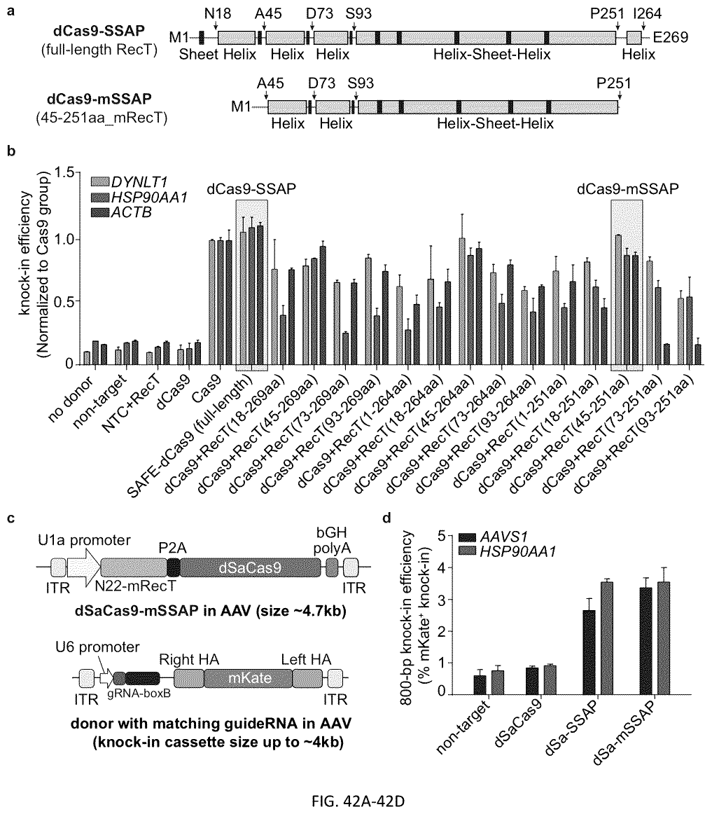

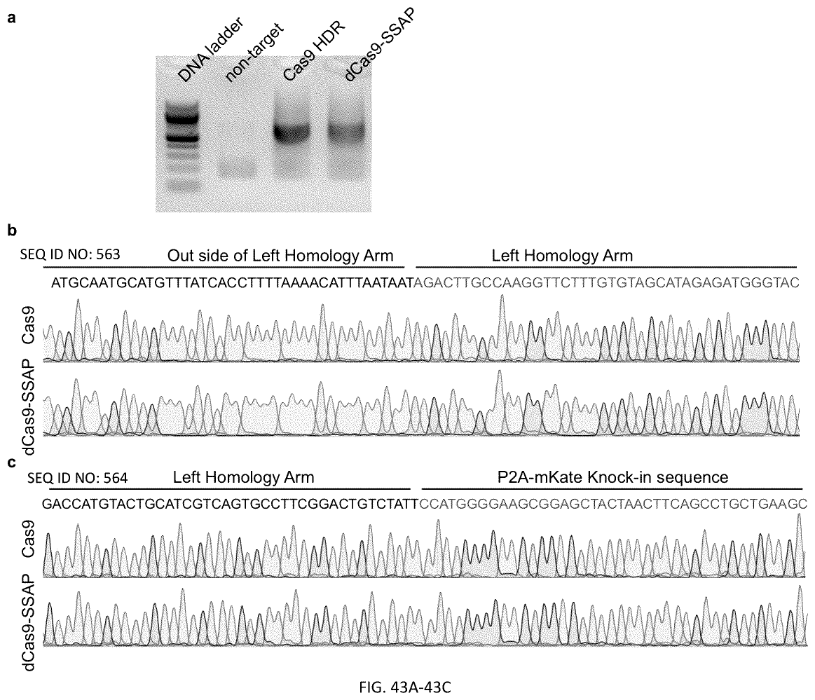

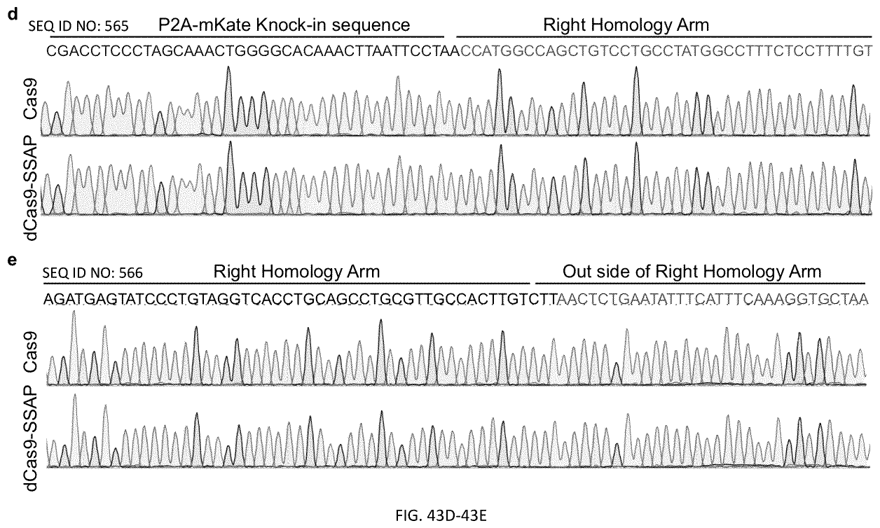

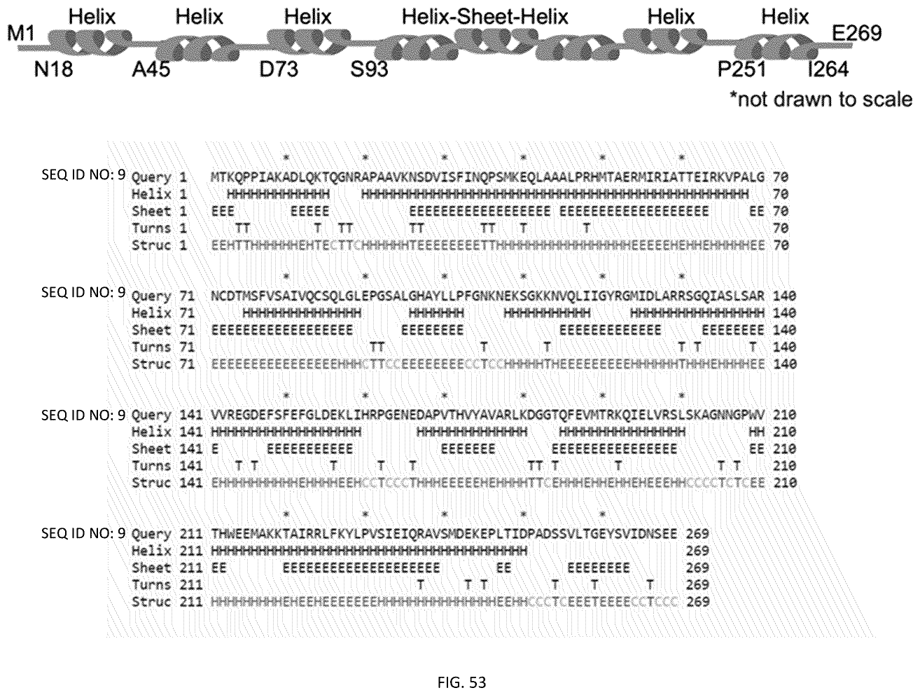

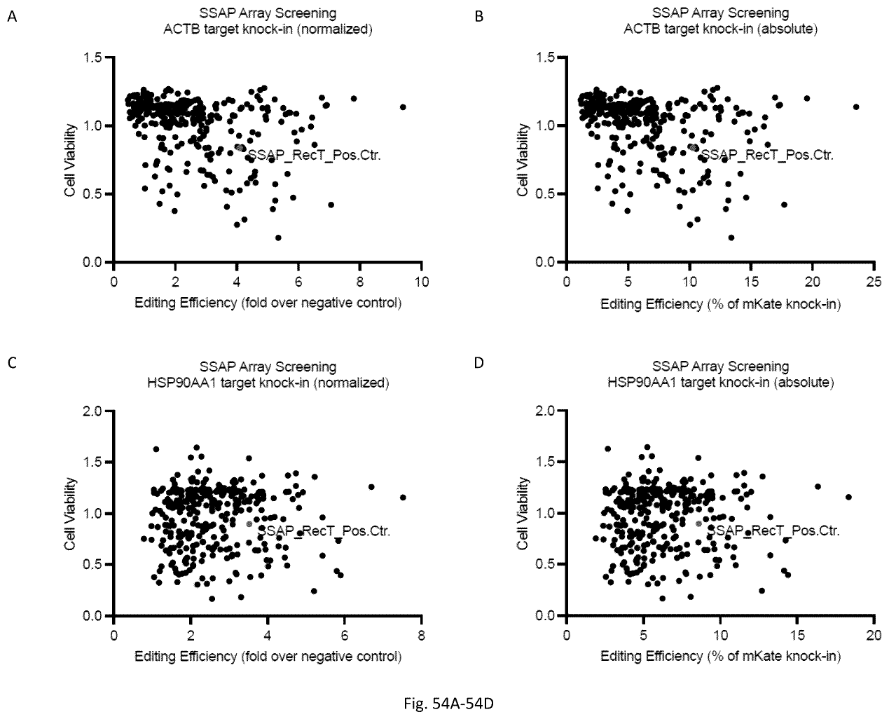

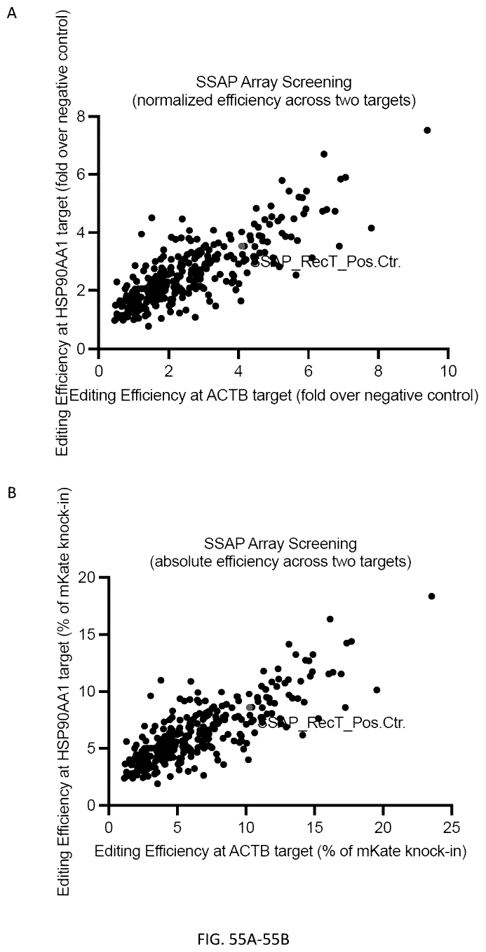

Finally, to optimize the dCas9-SSAP editor for potential future applications, we sought to develop a minimal version compatible with the size limitations of viral vectors such as AAV. We designed 14 different truncated EcRecT variants based on its secondary structure prediction (FIG. 42A, FIG. 53), and tested all constructs for their gene-editing activities alongside full-length dCas9-SSAP controls. From the optimization results, we identified a short RecT variant (around ˜200aa in length) that had comparable efficiencies with the original full-length RecT-based design (FIG. 42B).

We next integrated this short RecT variant with the more compact SaCas9 system and the smaller N22-BoxB aptamer design to build a minimal-functional dSaCas9-mSSAP editor (FIG. 42C). This allowed us to fit the dSaCas9-mSSAP into a single AAV and employ a >=4 kb donor AAV for long-sequence editing (FIG. 42C). We tested the dSaCas9-mSSAP editor via delivery of AAV2 particles, and confirmed that it had efficiencies comparable to the full-length version in HEK293T cells (FIG. 42D). This design, while needing further in vivo validation, could provide a convenient option for the delivery of this dCas9 knock-in editor.

### Discussion

Overall, the dCas9-SSAP editor harmonizes the RNA-guided programmability of CRISPR genome-targeting with the SSAP activity of phage enzyme RecT. It enables long-sequence editing with minimal DNA damage and provides research and therapeutic possibilities for addressing some of the currently intractable diseases involving large disease-causing variants, delivering therapeutic genes in vivo where selection methods are limited, or minimizing undesirable modifications during gene-editing. Compared with other long-sequence editing methods that depend on endogenous repair pathways following DNA cleavage, dCas9-SSAP and its mini-version facilitate homology-mediated gene editing via non-cutting dCas9s. This efficient, low-error technology offers a new and complementary approach to existing CRISPR editing tools.

### Materials and Methods

### Plasmids Construction

Human codon optimized DNA fragments were ordered from Genescript, Genewiz and IDT DNA. The fragments encoding the recombination enzymes were Gibson assembled into backbones (addgene plasmid #61423) using Q5® High-Fidelity 2× Master Mix (New England BioLabs). The amino acids sequence for these SSAP could be found in the Table 8. All sgRNAs were inserted into backbones (dCas9-SSAP and dSaCas9-SSAP plasmids) using Golden Gate cloning. dCas9-SSAP plasmids bearing BbsI (dSpCas9) and BsaI (dSaCas9) sites as gRNA backbones were sequence-verified (Eton and Genewiz). The sgRNA sequence used in this research could be found in the Table 6. All dCas9-SSAP plasmids will be deposited to Addgene for open access.

### Cell Culture

Human Embryonic Kidney (HEK) 293T, Hela, HepG2 and U2OS cells were maintained in Dulbecco's Modified Eagle's Medium (DMEM, Life Technologies), with 10% fetal bovine serum (FBS, BenchMark), 100 U/mL penicillin, and 100 μg/mL streptomycin (Life Technologies) at 37° C. with 5% CO₂. HEK 293T, Hela, HepG2 and U2OS cells were obtained from American Type Culture Collection (ATCC). The identity of the cell line is authenticated regularly by short tandem repeat (STR) assay and routinely tested for the presence of Mycoplasma using qPCR assay.

hES-H9 cells were maintained in mTeSR1 medium (StemCell Technologies) at 37° C. with 5% CO₂. Culture plates were pre-coated with Matrigel (Corning) 12 hours prior to use. 10 μM Rho Kinase inhibitor Y27632 (Sigma) was added for the first 24 hours after each passaging. Culture media was changed every 24 hours.

### Transfection

HEK293T, Hela, HepG2 and U2OS cells were seeded into 96-well plates (Corning) 12-24 hours prior to transfection at a density of 30,000 cells/well, and 250 ng of total DNA was transfected per well. Cells were transfected with Lipofectamine 3000 (Life Technologies) following the manufacturer's instructions when the cell are ˜70% confluence. In brief, we used 250 ng total DNA, 0.4 ul Lip3000 reagent, mixed with 10 ul of Opti-MEM per well. For the 250 ng DNA, we used 160 ng of dCas9-SSAP guide RNA plasmids (for double sgRNAd design, use equal amount of the two guide RNA plasmids, e.g., 80 ng each), 60 ng of pMCP-RecT or GFP control plasmid (addgene #64539) and 30 ng of PCR template DNA (the PCR primer could be found in Table 7, the template sequence could be found in Supplementary Sequences). Three days later, the cells were analyzed using FACS.

### Electroporation

For hES-H9 transfection, P3 Primary Cell 4D-Nucleofector™ X Kit S (Lonza) was used following the manufacturer's protocol. In brief, the hES-H9 cells were resuspended using Accutase (Innovative Cell Technology) and washed with PBS twice before the electroporation. For each reaction, 300,000 cells were nucleofected with 4 μg total DNA mixed in 20 ul electroporation buffer using the DC100 Nucleofector Program. For the 4 ug DNA, we used 2.6 μg of dCas9-SSAP guide RNA plasmids (for double sgRNAd design, use equal amount of the two guide RNA plasmids, e.g., 1.3 ug each), 1 μg of pMCP-RecT or GFP control plasmid and 0.4 ug of PCR template DNA (the PCR primer could be found in Table 7, the template sequence could be found in Supplementary Sequences). After electroporation, the cells were seeded into 12-well plates with 1 mL of mTeSR1 media added with 10 uM Y27632. Culture media was changed every 24 hours. Four days later, the cells were analyzed using FACS.

### Fluorescence-Activated Cell Analysis (FACS)

mKate knock-in efficiency was analyzed on a CytoFLEX flow cytometer (Beckman Coulter; Stanford Stem Cell FACS Core). 72 hours after transfection or 96 hours after electroporation, cells were washed twice with PBS and dissociated with TrypLE Express Enzyme (Thermo Fisher Scientific). Cell suspension was then transferred to a 96-well U-bottom plate (Thermo Fisher Scientific) and centrifuged at 300 g for 5 minutes. After removing the supernatant, pelleted cells were resuspended with 50 μl 4% FBS in PBS, and cells were analyzed within 30 minutes after preparation.

### Sanger Sequencing and NGS of Knock-In Junctions

HEK293T cells transfected with plasmid DNA and HDR templates were harvested 72 hours after transfection. The genomic DNA of these cells were extracted using the QuickExtract DNA Extraction Solution (Biosearch Technologies) following the manufacturer's protocol. The target genomic region was amplified using specific primers outside of the homology arms of the HDR template. The primers used for Sanger sequencing or NGS analysis could be found in the Table 7. PCR products were purified with Monarch PCR & DNA Cleanup Kit (New England BioLabs). 100 ng of purified product was sent for Sanger sequencing with target-specific primers (EtonBio or Genewiz).

Treatment with HR and Cell Cycle Inhibitor

All inhibitors were ordered from Sigma-Aldrich. For different inhibitor assays, the cells were pretreated with Mirin (Sigma, M9948-5 MG, 25 uM), B02 (Sigma, SML0364, 10 uM),) or RI-1 (Sigma, 553514-10 MG-M, 1 uM) for 16 hours. For cell cycle test, the cells were pretreated with Thymidine (Sigma, T9250-1G, 2 mM) for 18 hours, then remove thymidine, culture the cells using normal D10 without thymidine for 9 hours, add the second round of thymidine to a final concentration of 2 mM for another 18 hours. After the inhibitor and thymidine, the cells were transfected with dCas9-SSAP using Lipofectamine 3000 following the manufacturer's instruction. 3 days later, the cells were analyzed on a CytoFLEX flow cytometer and genomic DNA were also harvested for sequencing validation as above.

### Next-Generation Sequencing Library Preparation

72 hours after transfection, genomic DNA was extracted using QuickExtract DNA Extraction Solution (Biosearch Technologies). 200 ng total DNA was used for NGS library preparation. Genes of interest were amplified using specific primers (Table 7) for the first round PCR reaction. Illumina adapters and index barcodes were added to the fragments with a second round PCR using the primers listed in Table 7. Round 2 PCR products were purified by gel electrophoresis on a 2% E-gel using the Monarch DNA Gel Extraction Kit (New England BioLab). The purified product was quantified with Qubit dsDNA HS Assay Kit (Thermo Fisher) and sequenced on an Illumina MiSeq system using paired-end PE300 kits. All sequencing data will be deposited to NCBI SRA archive.

### TOPO Cloning Experiment

Total of 250 ng genomic DNA was used for the TOPO cloning experiments. The knock-in events were amplified using specific TA colony primers targeted to DYNLT1 or HSP90AA1 locus (Table 7) using Phusion Flash High-Fidelity PCR Master Mix (ThermoScientific, F-548L). Purify the targeted PCR products using Gel extraction kit (New England BioLabs, T1020L) following the manufacturer's instructions. Add a-tail to the PCR products using Taq polymerase (New England BioLabs, M0273S) through incubate at 72 C for 30 minutes. Set up the TOPO cloning reaction and transformation following the manufacturer's instructions (Thermo Scientific, K457501). Send the colony plates for RCA/colony sequencing using M13F (5′-GTAAAACGACGGCCAG-3′) [SEQ ID NO: 560] and M13R (5′-CAGGAAACAGCTATGAC-3′) [SEQ ID NO: 561] primers. The sequence results were analyzed using SnapGene software.

### High-Throughput Sequencing Data Analysis

Processed (demultiplexed, trimmed, and merged) sequencing reads were analyzed to determine editing outcomes using CRISPPResso2 by aligning sequenced amplicons to reference and expected HDR amplicons. The quantification window was increased to 10 bp surrounding the expected cut site to better capture diverse editing outcomes, but substitutions were ignored to avoid inclusion of sequencing errors. Only reads containing no mismatches to the expected amplicon were considered for HDR quantification; reads containing indels that partially matched the expected amplicons were included in the overall reported indel frequency. The computation work was supported by the SCG cluster hosted by the Genetics Bioinformatics Service Center (GBSC) at the Department of Genetics of Stanford. All customized scripts for data analysis will be deposited to Github under Cong Lab and made available for download.

### Insertion Site Mapping and Analysis

We are using a process that was previously developed (GIS-seq) and adapted for the genome-wide, unbiased off-target analysis of mKate knock-in, following the similar protocol in our previous study. Briefly, we harvest the HEK293T cells 3 days after transfection. The genomic DNA was size-selected to avoid the template contamination in the following step via the DNAdvance genomic DNA kit (A48705, Beckman Coulter). 400 ng of purified genomic DNA was fragmented to an average of 500 bp using NEB Fragmentase, ligated with adaptors, and size-selected using NEBNext Ultra II FS DNA Library Prep kit following manufacture's instruction. Following two rounds of nested anchored PCR to amplify targeted DNA (from the end of the knock-in sequence to the ligated adaptor sequence), and do a size-selected purification following the NEBNext Ultra II FS DNA library Prep kit protocol. The libraries were sequenced using Illumina Miseq V3 PE600 kits. Sequencing data was analyzed to determine off-target insertion events with all analysis code deposited to Github (github.com/cong-lab).

### Statistical Analysis

Unless otherwise stated, all statistical analysis and comparison were performed using t-test, with 1% false-discovery-rate (FDR) using a two-stage step-up method of Benjamini, Krieger and Yekutieli. All experiments were performed in triplicates unless otherwise noted to ensure sufficient statistical power in the analysis.

### SSAP Mining Process

For initial SSAP screening, we identified the three major family of phage recombination enzymes from Bacteriophage lambda, E. coli Rac prophage, and bacteriophage T7, and extracted the primary enzyme sequences as listed in supplementary sequences.

For RecT-like SSAP mining. RefSeq non-redundant protein database was downloaded from NCBI on Oct. 29, 2019. We systematically searched the NCBI non-redundant sequence database for RecT homologs. Our search follows two guidelines: 1) Closely-related candidates are less likely to have differential activities; 2) Microbial enzymes that function well when heterologous expressed in eukaryotic cells are difficult to predict, thus sampling diverse evolutionary branches of RecT homologs would be ideal. After identifying a large set of 2,071 candidates, we built phylogenetic trees and selected representative candidates after filtering out proteins with high sequence homology. Then, we used a threshold of at least 10% sequence divergence and sizes up to 300-aa (to avoid extremely large proteins that are hard to synthesize and less portable) to refine the hits, and randomly sampled the evolutionary branches to obtain a final list of 16 SSAPs (FIG. 38E, FIG. 44). Overall, the SSAP candidates have significant evolutionary and sequence heterogeneity, while retaining conserved regions that have been previously suggested to be important for their biochemical activities.

The multiple sequence alignment between RecT homologs used online tool (T-Coffee: tcoffcc.crg.cat/apps/tcoffee/do: regular).

Donor Design Test Comparing Cas9 HDR, Cas9 MMEJ, and dCas9-SSAP

As shown in FIG. 38F, we tested the new editor with different donor DNA designs. We considered three major types of donor DNAs with different homology arm (HA) length designs. Specifically, we synthesized: 1) HDR donors bearing long HAs (>=100 bp), a standard format for long-sequence engineering and transgene knock-in; 2) MMEJ donors with typically short HAs (<=50 bp), which have been shown to improve editing efficiencies for DSB-mediated knock-in; 3) NHEJ donors without HAs (Obp), which could help gauge the levels of donor integration due to Cas9-induced DSBs. Our results from these tests revealed two characteristics of dCas9-SSAP that are distinct from Cas9 gene-editing.

Firstly, for the NHEJ donors without any HAs (highlighted box in FIG. 38F), we observed knock-in cassette expression when using Cas9 editor but not for the dCas9-SSAP editor (FIG. 38F). This is consistent with previous reports that Cas9-mediated DSBs could induce NHEJ-mediated donor DNA insertion, but this integration is minimal when using the non-cutting dCas9-SSAP (FIG. 38F, dCas9-SSAP with NHEJ Obp donor).

Secondly, dCas9-SSAP benefited from successively longer HA within the donor, regardless of whether the HAs are for HDR-type or MMEJ-type, in contrast to Cas9 editor that showed a boost of knock-in efficiencies when using the MMEJ donors (FIG. 38F, HDR and MMEJ donors). This is consistent with the assumption that the enhancing effect when using MMEJ donors is dependent on Cas9 cleavage of target genomic sites.

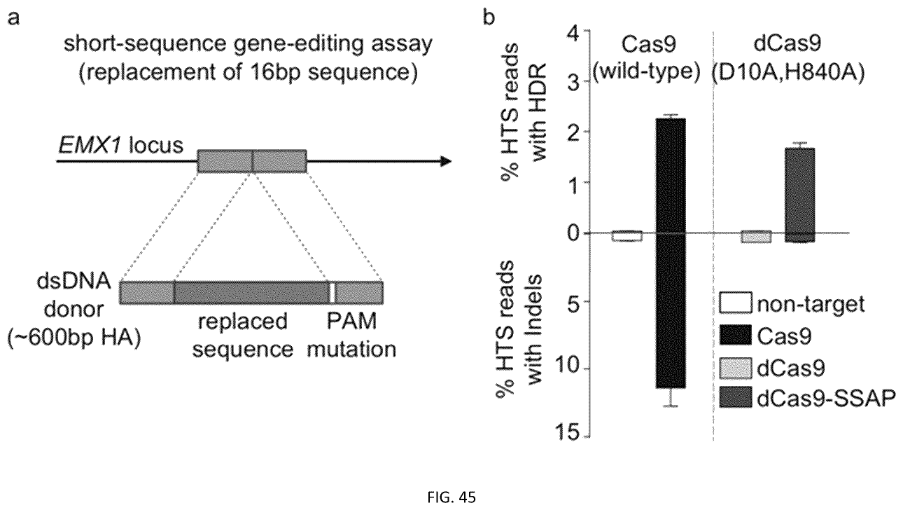

Further, while the focus of this work is long-sequence engineering, we also tested dCas9-SSAP for shorter sequence editing (FIG. 45) and observed precise knock-in of 16-bp sequence into EMX1 locus in human HEK293T cells. This experiment allowed us to verify the minimal indel formation when using dCas9-SSAP compared with Cas9-based editor using deep sequencing (FIG. 45B).

In summary, dCas9-SSAP editing becomes most efficient when using HDR donors, and longer homology arms in general make editing efficiency higher.

Step-by-Step Gene-Editing Protocol Using dCas9-SSAP Plasmids

### A. Design of Guide RNA Sequences at Target Genomic Loci

This step is the same as standard Cas9 experiments. Briefly, based on the Cas9 enzyme used, target sequence (usually 20-bp) near the knock-in or editing sites can be selected next to the protospacer adjacent motif (PAM). For SpCas9 use “NGG” and for SaCas9 use “NNGRRT”. We usually append extra “G” base to the beginning of the guide sequence to facilitate U6/Pol-III transcription initiation if the first base of the guide sequence is not “G”. Two DNA oligos could be ordered based on selected guides, with golden gate cloning overhangs, as shown below.

5′-CACCGNNNNNNNNNNNNNNNNNNN-3′
3′-CNNNNNNNNNNNNNNNNNNNCAAA-5′

N denotes the guide sequences. Standard desalting oligos are sufficient for this cloning. The two oligos above will be annealed to form the insert fragments in the next step.

B. Annealing of Two DNA Oligos for Each Guide RNA Target. Perform Phosphorylation and Annealing of Each Pair of Oligos Via Reaction Setup Below.

| | | |
|---|---|---|
| oligo1 Top (100 uM) | 1 | ul |
| oligo2 Bottom (100 uM) | 1 | ul |
| 10X T4 ligation Buffer(NEB) | 1 | ul |
| ddH2O | 6.5 | ul |
| T4 PNK (NEB) | 0.5 | ul |
| Total | 10 | ul |

Anneal in a thermocycler using the following parameters:

| | |
|---|---|
| 37 C. | 30 min |
| 95 C. | 5 min and then ramp down to 25° C. at 5 C./min |

C1. Golden Gate Cloning of Annealed Oligos into sgRNA/dspCas9 (dCas9-SSAP) Plasmid

For wild-type Cas9 test, one guide RNA is needed and the backbone vectors for the cloning will bear BbsI cloning sites matching the annealed oligos from Step B. The wild-type Cas9 plasmids for this step will be: pCas9-MS2-BB_BbsI (see list of plasmids at end of protocol)

| Item | Volume | Note |
|---|---|---|
| Water | 4.3 ul | |
| Cutsmart Buffer | 0.8 ul | 10x |
| T4 ligase | 0.2 ul | |
| BbsI-HF | 0.4 ul | |
| ATP (25 mM) | 0.3 ul | ~final 1 mM |
| plasmid/vector | 1 ul | ~50 ng total plasmid |
| Annealed Oligo (1:10 diluted) | 1 ul | diluted 10 ul into 100 ul |
| Total | 8 ul | |

This protocol uses a minimal amount of enzyme and could be scaled up as needed. After setting up the golden gate reaction (on ice), immediately move the reaction into Thermocycler and perform the golden gate reaction using the following parameters:

| | |
|---|---|
| 37 C. | 5 min |
| 16 C. | 5 min |

cycle for ~25 cycles, additional cycles up to 50 could be used
to maximize efficiency

| | |
|---|---|
| 65 C. | 5 min |
| 4 C. | hold |

After the reaction, perform bacterial transformation as per standard protocol of the competent cells used in the lab.

C2. Golden Gate Cloning of Annealed Oligos into sgRNA/dspCas9 (dCas9-SSAP) Plasmid

For dCas9-SSAP using dSpCas9, one or two guide RNAs can be used with double guide RNAs providing slightly better efficiency of editing. The backbone vectors for the cloning will bear BbsI cloning sites matching the annealed oligos from Step B. The dCas9-SSAP plasmids for this step will be: pdCas9-SSAP-MS2-BB_BbsI (see list of plasmids at end of protocol)

| Item | Volume | Note |
|---|---|---|
| Water | 4.3 ul | Add first |
| Cutsmart Buffer | 0.8 ul | 10x |
| T4 ligase | 0.2 ul | |
| BbsI-HF | 0.4 ul | |
| ATP (25 mM) | 0.3 ul | ~final 1 mM |
| dCas9-SSAP plasmid/vector | 1 ul | ~50 ng total p-dCas9-SSAP |
| | | plasmid |
| Annealed Oligo (1:10 diluted) | 1 ul | diluted 10 ul into 100 ul |
| Total | 8 ul | |

Golden Gate reaction setup and transformation steps are similar as above.

### D. Preparation of HDR Templates

Please refer to Supplementary Sequences for template used in the study and examples of template designs are illustrated as in FIG. 38. We recommend using a dsDNA template with at least 200 bp of homology arms on each end of the insertion/replacement sequences (the edited portion of the template). We suggest cloning the template into simple plasmids such as pUC19, then, restriction digestion of plasmids or standard PCR (using primers such as listed in the Table 7) could be employed for generating large amounts of dsDNA templates.

E. Perform Gene-Editing Via Delivery of dCas9-SSAP Plasmids and Template DNA

With previous steps, the three components of dCas9-SSAP editing method are ready for experiments: the guide RNA/Cas9 plasmid (cloned in step A-C), the template DNA (from step D), and the SSAP plasmid (pMCP-RecT, can be obtained from Addgene). For delivery into cells in vitro, routine transfection or electroporation could be performed following the recommended conditions by the reagent or equipment manufacturer and selected based on the cell types. For HEK293T cells as an example, a typical transfection condition is described below:
1. One day before transfection, 3E4 HEK293T/Hela/HepG2/U2OS cells seeded on each well of 96-well plate, the cell density should be around 70% on the next day at the time of transfection.
2. For lipofectamine 3000 as the transfection reagent, use a total of 250 ng DNA+0.4 ul Lip3000 reagents (ea.) and perform the reagent set up using 10 ul of Opti-MEM per well, as in the manufacturer's protocol.
3. Transfection material:
   dCas9-SSAP guide RNA plasmids, 160 ng (for double sgRNAd design, use equal amount of the two guide RNA plasmids, e.g., 80 ng each); pMCP-RecT or GFP control plasmid, 60 ng; Template DNA, up to 30 ng.
4. Mix plasmids with template DNA and perform transfection according to the manufacturer's protocol for HEK293T/Hela/HepG2/U2OS cells.
5. 12-24 hours after transfection, if applicable could switch to fresh media.
6. After at least 3 days post transfection, cells could be harvested or proceed to downstream experiments or analysis as needed.

List of Plasmids (all will be available at Addgene via plasmid ID)

| Plasmid ID | Detailed Description |
|---|---|
| SpCas9 Plasmids | |
| pCas9-MS2-BB_BbsI | pU6-MS2-gRNA-backbone(BbsI)- |
| | CBH-SpCas9-T2A-EBFP |
| pMCP-RecT | pLenti-EF1A-MCP-EXTEN-RecT- |
| | NLS |
| dCas9-SSAP Plasmids | |
| p-dCas9-SSAP-MS2-BB_BbsI | pU6-MS2-gRNA-backbone(BbsI)- |
| | CBH-dSpCas9-T2A-EBFP |
| pMCP-RecT | Same as above |
| dsaCas9-SSAP plasmids | |
| p-dSaCas9-SSAP-boxB-BB_BsaI | pU1aN22-miniRecT-p2A-dSaCas9 |
| p-dSa-SSAP-template | pU6-boxB-BB-H1-boxB-BB-Right |
| | HA-mKate-Left HA |

Sequences for gRNAs are provided in Table 6. Annotations of the guide RNA names are: guides starting with sp indicate SpCas9 guide RNA targets, and guides starting with dsp indicate dSpCas9 guide RNA targets.

TABLE 6
Sequence for gRNAs

| | Genomic | |
|---|---|---|
| guideRNA Name | target | Guide sequence |
| sp-DYNLT1 | DYNLT1 | AAGGCCATAGGCTGGACTGC (SEQ ID NO: 23) |
| sp-HSP90AA1 | HSP90AA1 | GTAGACTAATCTCTGGCTGA (SEQ ID NO: 24) |
| sp-ACTB | ACTB | CCACCGCAAATGCTTCTAGG (SEQ ID NO: 492) |
| dsp-DYNLT1-guide1 | DYNLT1 | AAGGCCATAGGCTGGACTGC (SEQ ID NO: 29) |
| dsp-DYNLT1-guide2 | DYNLT1 | GGCACTGACGATGCAGTACA (SEQ ID NO: 30) |
| dsp-HSP90AA1-guide1 | HSP90AA1 | GTAGACTAATCTCTGGCTGA (SEQ ID NO: 31) |
| dsp-HSP90AA1-guide2 | HSP90AA1 | TCGTCATCTCCTTCAAGGGG (SEQ ID NO: 32) |
| dsp-OCT4-guide1 | OCT4 | ATGCATGGGAGAGCCCAGAG (SEQ ID NO: 33) |
| dsp-OCT4-guide2 | OCT4 | GCCTGCCCTTCTAGGAATGG (SEQ ID NO: 34) |
| dsp-ACTB-guide1 | ACTB | CCACCGCAAATGCTTCTAGG (SEQ ID NO: 492) |
| dsp-ACTB-guide2 | ACTB | GCTTGCTGATCCACATCTGC (SEQ ID NO: 493) |
| dSa-HSP90AA1- | HSP90AA1 | AGTAGACTAATCTCTGGCTG (SEQ ID NO: 494) |
| guide1 | | |
| dSa-HSP90AA1- | HSP90AA1 | GATGTGTCGTCATCTCCTTCA (SEQ ID |
| guide2 | | NO: 495) |
| dSa-AAVS1-guide1 | AAVS1 | CACAGTGGGGCCACTAGGGA (SEQ ID |
| | | NO: 496) |
| dSa-AAVS1-guide2 | AAVS1 | GAGCCACATTAACCGGCCCT (SEQ ID |
| | | NO: 497) |
| dSp-CLTA-guide1 | CLTA | CGGATCCAGCTCAGCCATGG (SEQ ID |
| | | NO: 498) |
| dSp-CLTA-guide2 | CLTA | GGCGGTCCCGCGCTGGGGAA (SEQ ID |
| | | NO: 499) |
| dSp-RAB11A-guide | RAB11A | GGTAGTCGTACTCGTCGTCG (SEQ ID NO: 500) |
| dSp-HIST1H2BK- | HIST1H2BK | TGGGCTTTAAGACGCTTACT (SEQ ID NO: 501) |
| guide1 | | |
| dSp-HIST1H2BK- | HIST1H2BK | TAAACACGTGTTCCTAATCC (SEQ ID NO: 502) |
| guide2 | | |
| dSp-BCAP31-guide1 | BCAP31 | TGGACAAGAAGGAAGAGTAA (SEQ ID |
| | | NO: 503) |
| dSp-BCAP31-guide2 | BCAP31 | GACAGAAATCCCACAGCAGT (SEQ ID |
| | | NO: 504) |

Table 7 provides Primer Sequences.

Sequences for primers used for DNA template generation, targeted sequencing, and NGS assays are listed below. All NGS adapter sequences are shown underscored color.

TABLE 7
Primer Sequences

| | | Genomic | |
|---|---|---|---|
| Primer name | Usage | Target | Primer sequence |
| RecT-18 aa-F | Truncation | RecT | AATAGAGCCCCTGCTGCTGTGAAG |
| | | | (SEQ ID NO: 505) |
| RecT-45 aa-F | Truncation | RecT | GCCCTGCCTCGCCACATGA |
| | | | (SEQ ID NO: 506) |
| RecT-73 aa-F | Truncation | RecT | GATACCATGTCTTTCGTGAGCGCC |
| | | | (SEQ ID NO: 507) |
| RecT-93 aa-F | Truncation | RecT | AGTGCCCTGGGCCACGC |
| | | | (SEQ ID NO: 508) |
| RecT-251 aa-R | Truncation | RecT | AGGGTCGATGGTCAGGGGC |
| | | | (SEQ ID NO: 509) |
| RecT-264 aa-R | Truncation | RecT | AATCACAGAGTACTCGCCGGTCAG |
| | | | (SEQ ID NO: 510) |
| DYNLT1- | PCR | DYNLT1 | TGCCGTAAATGCTGCTCTCT |
| PCR-100 bp-F | template | | (SEQ ID NO: 39) |
| DYNLT1- | PCR | DYNLT1 | AGACTTGCCAAGGTTCTTTGTG |
| PCR-200 bp-F | template | | (SEQ ID NO: 40) |
| DYNLT1- | PCR | DYNLT1 | AGTGACCTGTGTAATTATGCAGAAG |
| PCR-400 bp-F | template | | (SEQ ID NO: 41) |
| DYNLT1- | PCR | DYNLT1 | TGAAAGTGCCACAAAACAAAGAGA |
| PCR-100 bp-R | template | | (SEQ ID NO: 42) |
| DYNLT1- | PCR | DYNLT1 | AAGACAAGTGGCAACGCAG |
| PCR-200 bp-R | template | | (SEQ ID NO: 43) |
| DYNLT1- | PCR | DYNLT1 | CGTTTATGATACTATGCAGACTATGAAGA |
| PCR-400 bp-R | template | | AC |
| | | | (SEQ ID NO: 511) |
| DYNLT1- | PCR | DYNLT1 | GGAGAATAAGACCATGTACTGC |
| PCR-50-F | template | | (SEQ ID NO: 512) |
| DYNLT1- | PCR | DYNLT1 | GAGGATGAACTAGAGACAAAAGG |
| PCR-50-R | template | | (SEQ ID NO: 513) |
| DYNLT1- | PCR | DYNLT1 | TCAGTGCCTTCGGACTG |
| PCR-25-F | template | | (SEQ ID NO: 514) |
| DYNLT1- | PCR | DYNLT1 | AAAGGCCATAGGCAGGAC |
| PCR-25-R | template | | (SEQ ID NO: 515) |
| DYNLT1- | PCR | DYNLT1 | ACTGTCTATTGGAAGCGGA |
| PCR-10-F | template | | (SEQ ID NO:516) |
| DYNLT1- | PCR | DYNLT1 | GGACAGCTGGTTAGGAATTAAG |
| PCR-10-R | template | | (SEQ ID NO:517) |
| mKate-PCR-0- | PCR | DYNLT1 | GGAAGCGGAGCTACTAACTT |
| F | template | | (SEQ ID NO:518) |
| mKate-PCR-0- | PCR | DYNLT1 | TTAGGAATTAAGTTTGTGCCCC |
| R | template | | (SEQ ID NO:519) |
| HSP90AA1- | PCR | HSP90AA1 | ATGAAGATGACCCTACTGCTGAT |
| PCR-100 bp-F | template | | (SEQ ID NO: 45) |
| HSP90AA1- | PCR | HSP90AA1 | TACTGTCTTGAAAGCAGATAGAAACC |
| PCR-200 bp-F | template | | (SEQ ID NO: 46) |
| HSP90AA1- | PCR | HSP90AA1 | GCAGCAAAGAAACACCTGGA |
| PCR-600 bp-F | template | | (SEQ ID NO: 47) |
| HSP90AA1- | PCR | HSP90AA1 | GTTGTCATGCCATACAGACTTTTT |
| PCR-100 bp-R | template | | (SEQ ID NO: 48) |
| HSP90AA1- | PCR | HSP90AA1 | AGCATTACTAGCTCTGCTTTAGTG |
| PCR-200 bp-R | template | | (SEQ ID NO: 49) |
| HSP90AA1- | PCR | HSP90AA1 | TCCACAAGACTGGGTCTGAG |
| PCR-600 bp-R | template | | (SEQ ID NO: 50) |
| HSP90AA1- | PCR | HSP90AA1 | AAATGCCACCCCTTGAAGG |
| PCR-50 bp-F | template | | (SEQ ID NO: 520) |
| HSP90AA1- | PCR | HSP90AA1 | ATCAGAGGAATTGTAGAGTACTGA |
| PCR-50 bp-R | template | | (SEQ ID NO: 521) |
| HSP90AA1- | PCR | HSP90AA1 | ACACATCACGCATGGAAGA |
| PCR-25 bp-F | template | | (SEQ ID NO: 522) |
| HSP90AA1- | PCR | HSP90AA1 | GGTAAGTCATCCCTCAGCC |
| PCR-25 bp-R | template | | (SEQ ID NO: 523) |
| HSP90AA1- | PCR | HSP90AA1 | AGAAGTAGACGGAAGCGG |
| PCR-10 bp-F | template | | (SEQ ID NO: 524) |
| HSP90AA1- | PCR | HSP90AA1 | AGCCACAGATTTAGGAATTAAGTTT |
| PCR-10 bp-F | template | | (SEQ ID NO: 525) |
| OCT4-PCR-F | PCR | OCT4 | GCGACTATGCACAACGAGAGG |
| | template | | (SEQ ID NO: 51) |
| OCT4-PCR-R | PCR | OCT4 | AAGTGTGTCTATCTACTGTGTCCCAG |
| | template | | (SEQ ID NO: 52) |
| ACTB-PCR-F | PCR | ACTB | TGTGGTGTGTGGGGAGCT |
| | template | | (SEQ ID NO: 526) |
| ACTB-PCR-R | PCR | ACTB | TTACACGAAAGCAATGCTATCACCTC |
| | template | | (SEQ ID NO: 527) |
| DYNLT1 KI | Junction | DYNLT1 | AGGAGGTCCCATCAGATGCT |
| PCR-F | PCR | | (SEQ ID NO: 59) |
| HSP90AA1 KI | Junction | HSP90AA1 | GGCTGGACAGCAAACATGGA |
| PCR-F | PCR | | (SEQ ID NO: 60) |
| Junction PCR | Junction | mKate | TTGCTGCCGTACATGAAGCTG |
| universial-R | PCR | | (SEQ ID NO: 62) |
| EMX1-NGS-F | NGS | EMX1 | CCATCTCATCCCTGCGTGTCTCCAGAAGA |
| | | | AGGGCTCCCATCAC |
| | | | (SEQ ID NO: 528) |
| EMX1-NGS-R | NGS | EMX1 | CCTCTCTATGGGCAGTCGGTGATGAGCA |
| | | | GCAAGCAGCACTCTG |
| | | | (SEQ ID NO: 529) |
| Junction NGS- | Junction | mKate | CCTCTCTATGGGCAGTCGGTGATGGTACA |
| 5′ common-R | NGS | | GCTTCATGTGCATGT |
| | | | (SEQ ID NO: 530) |
| Junction NGS- | Junction | mKate | CCATCTCATCCCTGCGTGTCTCCGAGGCC |
| 3′ common-F | NGS | | GACAAAGAGACA |
| | | | (SEQ ID NO: 531) |
| DYNLT1- | Junction | DYNLT1- | CCATCTCATCCCTGCGTGTCTCCGTAAAT |
| Junction NGS- | NGS | mKate | GCTGCTCTCTTCCC |
| 5′-F | | | (SEQ ID NO: 532) |
| DYNLT1- | Junction | DYNLT1- | CCTCTCTATGGGCAGTCGGTGATGTTGTG |
| Junction NGS- | NGS | mKate | AAAGTGCCACAAAACA |
| 3′-R | | | (SEQ ID NO: 533) |
| HSP90AA1- | Junction | HSP90AA1- | CCATCTCATCCCTGCGTGTCTCCCTACTG |
| Junction NGS- | NGS | mKate | CTGATGATACCAGTG |
| 5′-F | | | (SEQ ID NO: 534) |
| HSP90AA1- | Junction | HSP90AA1- | CCTCTCTATGGGCAGTCGGTGATGGTTGT |
| Junction NGS- | NGS | mKate | CATGCCATACAGACT |
| 3′-R | | | (SEQ ID NO: 535) |
| ACTB-TA-F | TA colony | ACTB | AGTCCTGTGGCATCCACGAA |
| | | | (SEQ ID NO: 536) |
| ACTB-TA-R | TA colony | ACTB | GGCACGAAGGCTCATCATTCAA |
| | | | (SEQ ID NO: 537) |
| HSP90AA1- | TA colony | HSP90AA1 | AGACACATGCTAACAGGATCTA |
| TA-F | | | (SEQ ID NO: 538) |
| HSP90AA1- | TA colony | HSP90AA1 | ATGCAGAATTGTCAACTACAGG |
| TA-R | | | (SEQ ID NO: 539) |
| HIST1H2BK- | PCR | HIST1H2BK | AGGCCATGGGAATCATGAAC |
| PCR-F | template | | (SEQ ID NO: 540) |
| HIST1H2BK- | PCR | HIST1H2BK | CTCACGTTAGAACGCCATTACA |
| PCR-R | template | | (SEQ ID NO: 541) |
| BCAP31-PCR- | PCR | BCAP31 | AGGCCTTTGGGTGCAGCTG |
| F | template | | (SEQ ID NO: 542) |
| BCAP31-PCR- | PCR | BCAP31 | CAGTCCACCTGCTCCACCC |
| R | template | | (SEQ ID NO: 543) |
| CLTA-PCR-F | PCR | CLTA | GGGTAGCTCCTGAACCATTGT |
| | template | | (SEQ ID NO: 544) |
| CLTA-PCR-F | PCR | CLTA | CCAGAAGCACTCAAACATGCTG |
| | template | | (SEQ ID NO: 545) |
| RAB11A- | PCR | RAB11A | CTGCCGGAAATGGCGCAG |
| PCR-F | template | | (SEQ ID NO: 546) |
| RAB11A- | PCR | RAB11A | GTAGGAGACGGAACCGCACAA |
| PCR-R | template | | (SEQ ID NO: 547) |

Table 8 provides sequence for certain SSAP tested in this Example.

TABLE 8
SSAP Sequences

| SSAP | Amino Acid Sequence |
|---|---|
| Lambda bet | MSTALATLAGKLAERVGMDSVDPQELITTLRQTAFKGDASDAQFIALLI |
| (SEQ ID | VANQYGLNPWTKEIYAFPDKQNGIVPVVGVDGWSRIINENQQFDGMDF |
| NO: 166) | EQDNESCTCRIYRKDRNHPICVTEWMDECRREPFKTREGREITGPWQSH |
| | PKRMLRHKAMIQCARLAFGFAGIYDKDEAERIVENTAYTAERQPERDIT |
| | PVNDETMQEINTLLIALDKTWDDDLLPLCSQIFRRDIRASSELTQAEAVK |
| | ALGFLKQKAAEQKVAA |
| Rac RecT | MTKQPPIAKADLQKTQGNRAPAAVKNSDVISFINQPSMKEQLAAALPRH |
| (SEQ ID | MTAERMIRIATTEIRKVPALGNCDTMSFVSAIVQCSQLGLEPGSALGHAY |
| NO: 167) | LLPFGNKNEKSGKKNVQLIIGYRGMIDLARRSGQIASLSARVVREGDEFS |
| | FEFGLDEKLIHRPGENEDAPVTHVYAVARLKDGGTQFEVMTRKQIELVR |
| | SLSKAGNNGPWVTHWEEMAKKTAIRRLFKYLPVSIEIQRAVSMDEKEPL |
| | TIDPADSSVLTGEYSVIDNSEE |
| T7 gp2.5 | MAKKIFTSALGTAEPYAYIAKPDYGNEERGFGNPRGVYKVDLTIPNKDP |
| (SEQ ID | RCQRMVDEIVKCHEEAYAAAVEEYEANPPAVARGKKPLKPYEGDMPFF |
| NO: 168) | DNGDGTTTFKFKCYASFQDKKTKETKHINLVVVDSKGKKMEDVPIIGGG |
| | SKLKVKYSLVPYKWNTAVGASVKLQLESVMLVELATFGGGEDDWADE |
| | VEENGYVASGSAKASKPRDEESWDEDDEESEEADEDGDF |
| CspRecT | MNQIVKFTDDSGLAVQVTPDDVRRYICENATEKEVGLFLQLCQTQRLNP |
| (SEQ ID | FVKDAYLVKYGGAPASMITSYQVFNRRACRDANYDGIKSGVVVLRDGD |
| NO: 169) | VVHKRGAACYKKAGEELIGGWAEVRFKDGRETAYAEVALDDYSTGKS |
| | NWAKMPGVMIEKCAKAAAWRLAFPDTFQGMYAAEEMDQAQQPEQVR |
| | AQAEQPVDLQPIRELFKPYCEHFGITPAEGMTAVCGAVGAEGMHSMTE |
| | QQARRARAWMEEEMAAPAVEAEYEVVDEGEVF |
| PapRecT | MGTALTPLLTKFATRYEMGTTPEEVANTLKQTCFKGQVNDSQMVALLI |
| (SEQ ID | VADQYKLNPFTKELYAFPDKNNGIVPVVGVDGWARIINENPQFDGMEFS |
| NO: 170) | MDQQGTECTCKIYRKDRSHAISATEYMAECKRNTQPWQSHPRRMLRHK |
| | AMIQCARLAFGFAGIYDQDEAERIVERDVTPAEQYEDVSEAICLIKDSPT |
| | MEDLQAAFSNAWKAYKTKGARDQLTAAKDQRKKELLDAPIDVEFEET |
| | GDDRAA |
| EcRecT | MTKQPPIAKADLQKTQGNRAPAAIKNNDVISFINQPSMKEQLAAALPRH |
| (SEQ ID | MTAERMIRIATTEIRKVPALGNCDTMSFVSAIVQCSQLGLEPGSALGHAY |
| NO: 171) | LLPFGNKNEKSGKKNVQLIIGYRGMIDLARRSGQIASLSARVVREGDEFN |
| | FEFGLDEKLIHRPGENEDAPVTHVYAVARLKDGGTQFEVMTRKQIELVR |
| | SQSKAGNNGPWVTHWEEMAKKTAIRRLFKYLPVSIEIQRAVSMDEKEPL |
| | TIDPADSSVLTGEYSVIDNSEE |
| PasRecT | MSNQPPIASADLQKANTGKQVANKTPEQTLVGFMNQPAMKSQLAAALP |
| (SEQ ID | RHMTADRMIRIVTTEIRKTPALATCDQSSFIGAVVQCSQLGLEPGSALGH |
| NO: 115) | AYLLPFGNGRSKSGQSNVQLIIGYRGMIDLARRSGQIVSLSARVVRADDE |
| | FSFEYGLDENLIHRPGENEDAPITHVYAVARLKDGGTQFEVMTVKQIEK |
| | VKAQSKASSNGPWVTHWEEMAKKTVIRRLFKYLPVSIEMQKAVILDEK |
| | AESDVDQDNASVLSAEYSVLDGSSEE |
| SeRecT | MTKQPPIAKADLQKTQENRAPAAIKNNDVISFINQPSMKEQLAAALPRH |
| (SEQ ID | MTAERMIRIATTEIRKVPALGNCDTMSFVSAIVQCSQLGLEPGSALGHAY |
| NO: 131) | LLPFGNKNEKSGKKNVQLIIGYRGMIDLARRSGQIASLSARVVREGDEFN |
| | FEFGLDEKLIHRPGENEDAPVTHVYAVARLKDGGTQFEVMTRRQIELVR |
| | SQSKAGNNGPWVTHWEEMAKKTAIRRLFKYLPVSIEIQRAVSMDEKEPL |
| | TIDPADSSVLTGEYSVIDNSEE |
| AcRecT | MTKQPPIAKADLQKTQGNRAPAAVNDKDVLCVINSPAMKAQLAAALPR |
| (SEQ ID | HMTAERMIRIATTEIRKVPELRNCDSTSFIGAIVQCSQLGLEPGSALGHAY |
| NO: 133) | LLPFGNGKAKNGKKNVQLIIGYRGMIDLARRSGQIISLSARVVRECDEFS |
| | YELGLDEKLVHRPGENEDAPITHVYAVAKLKDGGVQFEVMTKKQVEK |
| | VRDTHSKAAKNAASKGASSIWDEHFEDMAKKTVIRKLFKYLPVSIEIQR |
| | AVSMDGKEVETINPDDISVIAGEYSVIDNPEE |
| SejRecT | MNAPQKQNTRAAVKKISPQEFAEQFAAIIPQVKSVLPAHVTFEKFERVV |
| (SEQ ID | RLAVRKNPDLLTCSPASLFMACIQAASDGLLPDGREGAIVSRWSSKKSC |
| NO: 135) | NEASWMPMVAGLMKLARNSGDIASISSQVVFEGEHFRVVLGDEERIEHE |
| | RDLGKTGGKIVAAYAVARLKDGSDPIREIMSWGQIEKIRNTNKKWEWG |
| | PWKAWEDEMARKTVIRRLAKRLPMSTDKEGERLRSAIERIDSLVDISAN |
| | VDAPQIAADDEFAAAAHGVEPQQIAAPDLIGRLAQMQSLEQVQDIEPQV |
| | SHAIQEADKRGDSDTANALDAALQSALSRTSTAKEEVPA |
| PsaRecT | MPKQPPIAKADLQKTQGARTPTAVKNNNDVISFINQPSMKEQLAAALPR |
| (SEQ ID | HMTAERMIRIATTEIRKVPALGDCDTMSFVSAIVQCSQLGLEPGGALGH |
| NO: 137) | AYLLPFGNRNEKSGKKNVQLIIGYRGMIDLARRSGQIASLSARVVREGD |
| | DFSFEFGLEEKLVHRPGENEDAPVTHVYAVARLKDGGTQFEVMTRKQIE |
| | LVRAQSKAGNNGPWVTHWEEMAKKTAIRRLFKYLPVSIEIQRAVSMDE |
| | KETLTIDPADASVITGEYSVVENAGVEENVTA |
| PhRecT | MGHLVSKTEQDYIKQHYAKGATDQEFEHFIGVCRARGLNPAANQIYFV |
| (SEQ ID | KYRSKDGPAKPAFILSIDSLRLIAHRTGDYAGCSEPIFTDGGKACTVTVR |
| NO: 139) | RNLKSGETGNFSGMAFYDEQVQQKNGRPTSFWQSKPRTMLEKCAEAK |
| | ALRKAFPQDLGQFYIREEMPPQYDEPIQVHKPKALEEPRESKSDLSRRKG |
| | LNRKLSALGVDPSRFDEVATFLDGTPDRELGQKLKLWLKEAGYGVNQ |
| PraRecT | MNTDMIAMPPSPAISMLDTSKLDVMVRAAELMSQAVVMVPDHFKGKP |
| (SEQ ID | ADCLAVVMQADQWGMNPFTVAQKTHLVSGTLGYESQLVNAVISSSKAI |
| NO: 141) | KGRFHYEWSDGWERLAGKVQYVKESRQRKGQQGSYQVTVAKPTWKP |
| | EDEQGLWVRCGAVLAGEKDITWGPKLYLASVLVRNSELWTTKPYQQA |
| | AYTALKDWSRLYTPAVMQGSMTGKSWSLTGRLISPR |
| PabRecT | MSNQPPIASADLQKTQQSKQVANKTPEQTLVGFMNQPAMKSQLAAALP |
| (SEQ ID | RHMTADRMIRIVTTEIRKTPQLAQCDQSSFIGAVVQCSQLGLEPGSALGH |
| NO: 117) | AYLLPFGNGRSKSGQSNVQLIIGYRGMIDLARRSGQIVSLSARVVRADDE |
| | FSFEYGLDENLVHRPGENEDAPITHVYAVARLKDGGTQFEVMTVKQVE |
| | KVKAQSKASSNGPWVTHWEEMAKKTVIRRLFKYLPVSIEMQKAVVLDE |
| | KAESDVDQDNASVLSAEYSVLESGDEATN |
| PadRecT | MSNQPPLATADLQKTQQSNQVAKTPEQTLVGFMNQPAMKSQLAAALP |
| (SEQ ID | RHMTADRMIRIVTTEIRKTPALAQCDQSSFIGAVVQCSQLGLEPGSALGH |
| NO: 119) | AYLLPFGNGRSKSGQSNVQLIIGYRGMIDLARRSGQIVSLSARVVRADDE |
| | FSFEYGLDENLIHRPGDNESAPITHVYAVARLKDGGTQFEVMTAKQVEK |
| | VKAQSKASSNGPWVTHWEEMAKKTVIRRLFKYLPVSIEMQKAVVLDEK |
| | AESDVDQDNASVLSAEYSVLESGTGE |
| PlsRecT | MSNQPPIASADLQKTQQSKQVANKTPEQTLVGFMNQPAMKSQLAAALP |
| (SEQ ID | RHMTADRMIRIVTTEIRKTPALATCDQSSFIGAVVQCSQLGLEPGSALGH |
| NO: 121) | AYLLPFGNGRSKSGQSNVQLIIGYRGMIDLARRSGQIVSLSARVVRADDE |
| | FSFEYGLDENLIHRPGDNEDAPITHVYAVARLKDGGTQFEVMTAKQVEK |
| | VKAQSKASSNGPWVTHWEEMAKKTVIRRLFKYLPVSIEMQKAVVLDEK |
| | AESDVDQDNASVLSAEYSVLEGDGGE |
| PrsRecT | MSNPPLAQADLQKTQGTEVKEKTKDQMLVELINKPSMKAQLAAALPRH |
| (SEQ ID | MTPDRMIRIVTTEIRKTPALATCDMQSFVGAVVQCSQLGLEPGNALGHA |
| NO: 123) | YLLPFGNGKSKSGQSNVQLIIGYRGMIDLARRSGQIVSISARTVRQGDNF |
| | HFEYGLNENLTHVPGENEDSPITHVYAVARLKDGGVQFEVMTYNQIEK |
| | VRASSKAGQNGPWVSHWEEMAKKTVIRRLFKYLPVSIEMQKAVILDEK |
| | AEANIDQENATIFEGEYEEVGTDGK |
| PrRecT | MSNPPLAQSDLQKTQGTEVKVKTKDQQLIQFINQPSMKAQLAAALPRH |
| (SEQ ID | MTPDRMIRIVTTEIRKTPALATCDMQSFVGAVVQCSQLGLEPGNALGHA |
| NO: 125) | YLLPFGNGKAKSGQSNVQLIIGYRGMIDLARRSNQIISISARTVRQGDNF |
| | HFEYGLNEDLTHTPSENEDSPITHVYAVARLKDGGVQFEVMTYNQVEK |
| | VRASSKAGQNGPWVSHWEEMAKKTVIRRLFKYLPVSIEMQKAVVLDEK |
| | AEANVDQENATIFEGEYEEVGTDGN |
| ShpRecT | MKAQLAAALPKHITSDRMIRIVSTEIRKTPSLANCDIQSFIGAVVQCSQLG |
| (SEQ ID | LEPGNALGHAYLLPFGNGKSDNGKSNVQLIIGYRGMIDLARRSGQIISISA |
| NO: 143) | RTVRQGDNFHFEYGLNENLTHIPEGNEDSPITHVYAVARLKDEGVQFEV |
| | MTYNQIEKVRDSSKAGKNGPWVTHWEEMAKKTVIRRLFKYLPVSIEMQ |
| | KAVILDEKAEANIEQDHSAIFEAEFEEVDSNGN |
| BaRecT | MQTAQVKLSVPHQQVYQDNFNYLSSQVVGHLVDLNEEIGYLNQIVENS |
| (SEQ ID | LSTASPLDVAAPWSVYGLLLNVCRLGLSLNPEKKLAYVMPSWSETGEII |
| NO: 127) | MKLYPGYRGEIAIASNFNVIKNANAVLVYENDHFRIQAATGEIEHFVTSL |
| | SIDPRVRGACSGGYCRSVLMDNTIQISYLSIEEMNAIAQNQIEANMGNTP |
| | WNSIWRTEMNRVALYRRAAKDWRQLIKATPEIQSALSDTEY |
| ShsRecT | MSKQLTTVNTQAVVGTFSQAELDTLKQTIAKGTTNEQFALFVQTCANSR |
| (SEQ ID | LNPFLNHIHCIVYNGKEGATMSLQIAVEGILYLARKTDGYKGIECQLIHE |
| NO: 129) | NDEFKFDAKSKEVDHQIGFPRGNVIGGYAIAKREGFDDVVVLMESNEVD |
| | HMLKGRNGHMWRDWFNDMFKKHIMKRAAKLQYGIEIAEDETVSSGPS |
| | VDNIPEYKPQPRKDITPNQDVIDAPPQQPKQDDEAAKLKAARSEVSKKF |
| | KKLGIVKEDQTEYVEKHVPGFKGTLSDFIGLSQLLDLNIEAQEAQSADG |
| | DLLD |

### Template DNA Sequences

Annotations of the replaced or inserter editing sequences are detailed below with each of the templates. Unless otherwise noted, when different homology arms are used in the Example, we used primers listed in Table 7 to obtain templates with different homology arm lengths.

DYNLT1 P2A-mKate knock-in HDR template sequence
Left Homology Arm-Insertion Sequence-Right Homology Arm
(Underlined are the inserted mKate fluorescent protein sequence, the proceeding non-
underlined part is the P2A peptide sequence)
(SEQ ID NO: 548)

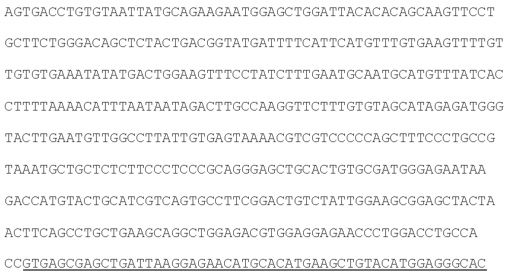

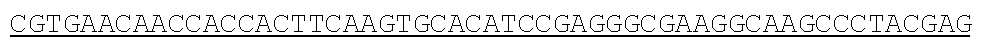

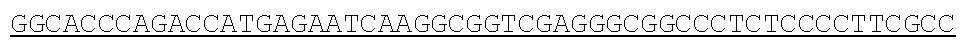

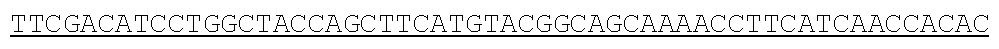

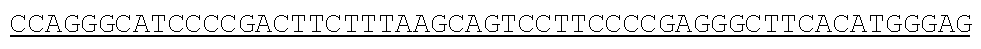

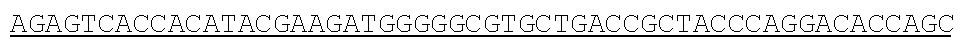

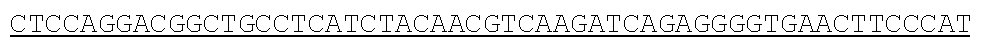

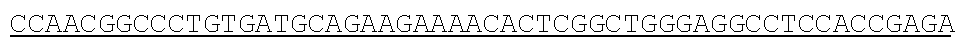

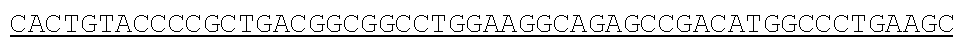

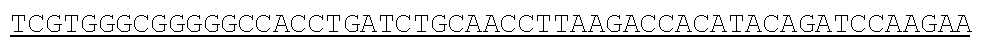

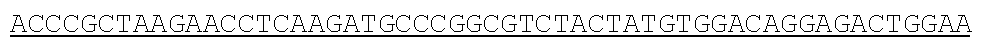

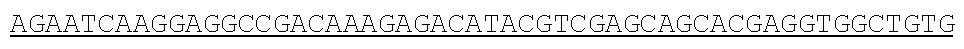

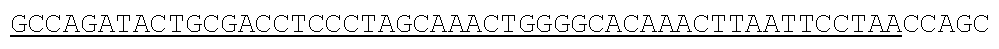

TGTCCtGCCTATGGCCTTTCTCCTTTTGTCTCTAGTTCATCCTCTAACCACCAGCCA
TGAATTCAGTGAACTCTTTTCTCATTCTCTTTGTTTTGTGGCACTTTCACAATGTAGA
GGAAAAAACCAATGACCGCACTGTGATGTGAATGGCACCGAAGTCAGATGAGTA
TCCCTGTAGGTCACCTGCAGCCTGCGTTGCCACTTGTCTTAACTCTGAATATTTCAT
TTCAAAGGTGCTAAAATCTGAAATCTGCTAGTGTGAAACTTGCTCTACTCTCTGAAA
TGATTCAAATACACTAATTTTCCATACTTTATACTTTTGTTAGAATAAATTATTCAAAT
CTAAAGTCTGTTGTGTTCTTCATAGTCTGCATAGTATCATAAACG
HSP90AA1 P2A-mKate knock-in HDR template sequence
Left Homology Arm-Insertion Sequence-Right Homology Arm
(Underlined are the inserted mKate fluorescent protein sequence, the proceeding non-
underlined part is the P2A peptide sequence)
(SEQ ID NO: 549)

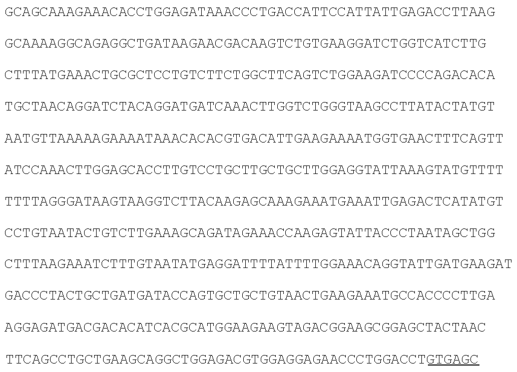

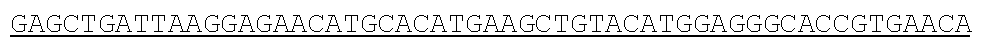

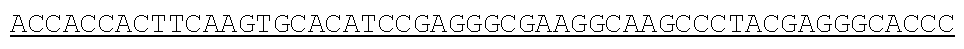

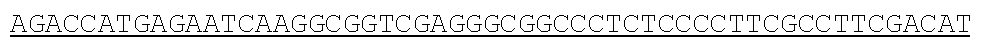

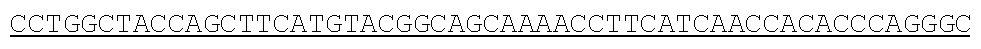

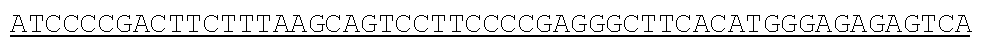

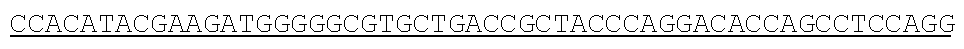

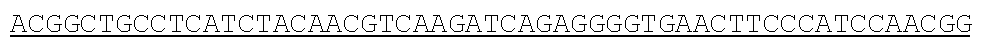

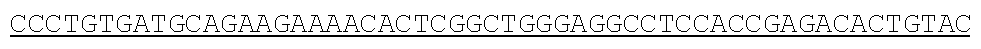

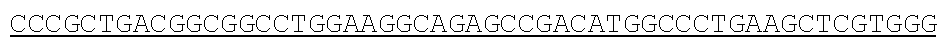

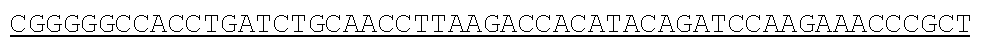

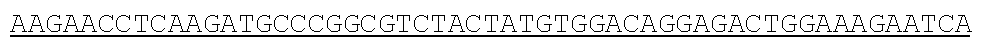

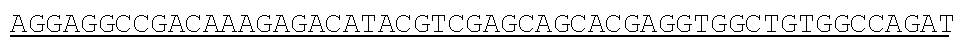

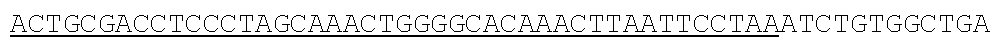

GGGATGACTTACCTGTTCAGTACTCTACAATTCCTCTGATAATATATTTTCAAGGAT
GTTTTTCTTTATTTTTGTTAATATTAAAAAGTCTGTATGGCATGACAACTACTTTAAG
GGGAAGATAAGATTTCTGTCTACTAAGTGATGCTGTGATACCTTAGGCACTAAAGC
AGAGCTAGTAATGCTTTTTGAGTTTCATGTTGGTTTATTTTCACAGATTGGGGTAAC
GTGCACTGTAAGACGTATGTAACATGATGTTAACTTTGTGGTCTAAAGTGTTTAGCT
GTCAAGCCGGATGCCTAAGTAGACCAAATCTTGTTATTGAAGTGTTCTGAGCTGTA
TCTTGATGTTTAGAAAAGTATTCGTTACATCTTGTAGGATCTACTTTTTGAACTTTTC
ATTCCCTGTAGTTGACAATTCTGCATGTACTAGTCCTCTAGAAATAGGTTAACTGA
AGCAACTTGATGGAAGGATCTCTCCACAGGGCTTGTTTTCCAAAGAAAAGTATTGT
TTGGAGGAGCAAAGTTAAAAGCCTACCTAAGCATATCGTAAAGCTGTTCAAAAATA
ACTCAGACCCAGTCTTGTGGA
AAVS1 P2A-mKate knock-in HDR template sequence
Left Homology Arm-Insertion sequence-RightHomology Arm
(Underlined are the inserted mKate fluorescent protein sequence, the proceeding non-
underlined part is the P2A peptide sequence)
(SEQ ID NO: 550)

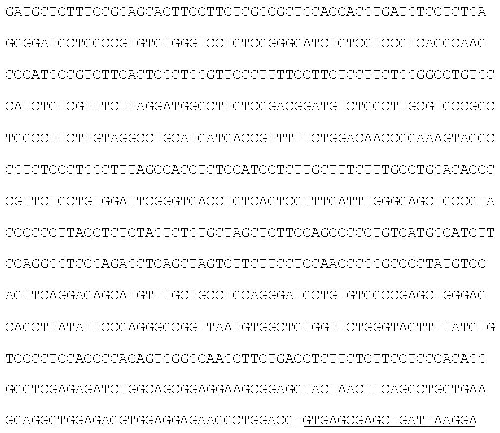

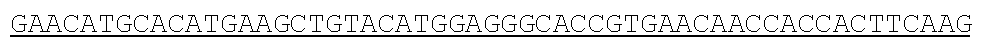

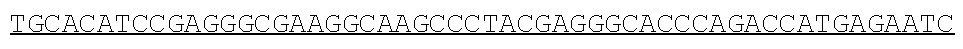

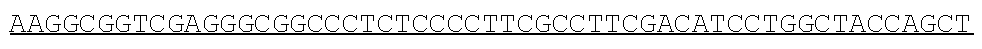

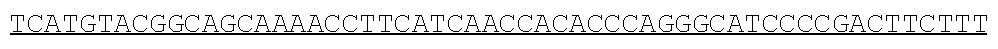

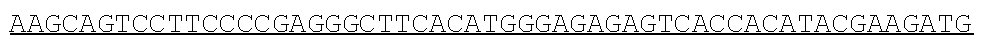

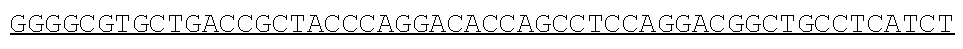

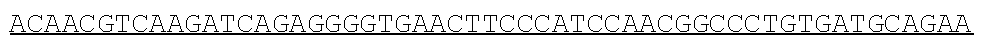

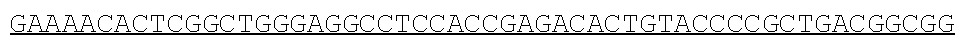

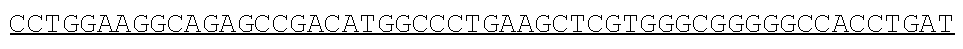

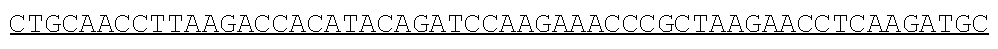

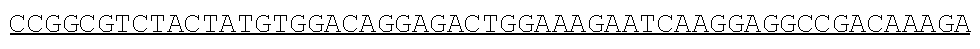

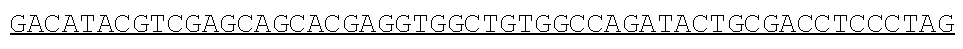

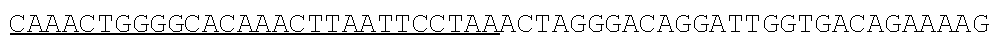

CCCCATCCTTAGGCCTCCTCCTTCCTAGTCTCCTGATATTGGGTCTAACCCCCACC
TCCTGTTAGGCAGATTCCTTATCTGGTGACACACCCCCATTTCCTGGAGCCATCTC
TCTCCTTGCCAGAACCTCTAAGGTTTGCTTACGATGGAGCCAGAGAGGATCCTGG
GAGGGAGAGCTTGGCAGGGGGTGGGAGGGAAGGGGGGGATGCGTGACCTGCCC
GGTTCTCAGTGGCCACCCTGCGCTACCCTCTCCCAGAACCTGAGCTGCTCTGACG
CGGCTGTCTGGTGCGTTTCACTGATCCTGGTGCTGCAGCTTCCTTACACTTCCCAA
GAGGAGAAGCAGTTTGGAAAAACAAAATCAGAATAAGTTGGTCCTGAGTTCTAACT
TTGGCTCTTCACCTTTCTAGTCCCCAATTTATATTGTTCCTCCGTGCGTCAGTTTTA
CCTGTGAGATAAGGCCAGTAGCCAGCCCCGTCCTGGCAGGGCTGTGGTGAGGAG
GGGGGTGTCCGTGTGGAAAACTCCCTTTGTGAGAATGGTGCGTCCTAGGTGTTCA
CCAGGTCGTGGCCGCCTCTACTCCCTTTCTCTTTCTCCATCCTTCTTTCCTTAAAGA
GTCCCCAGTGCTATCTGGGACATATTCCTCCGCCCAGAGCAGGGTCCCGCTTCCC
TAAGGCCCTGCTCTGGGCTTCTGGGTTTGAGTCCTTGGC
OCT4 P2A-mKate knock-in HDR template sequence
Left Homology Arm-Insertion Sequence-Right Homology Arm
(Underlined are the inserted mKate fluorescent protein sequence, the proceeding non-
underlined part is the P2A peptide sequence)
(SEQ ID NO: 551)

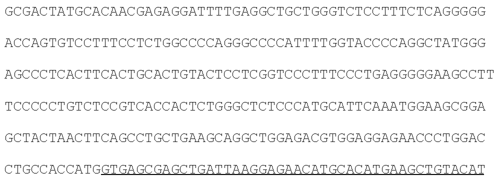

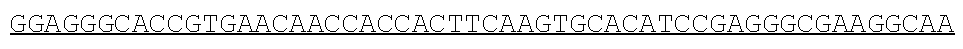

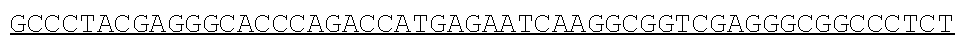

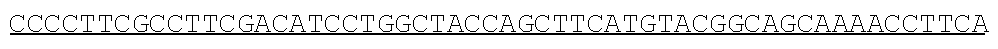

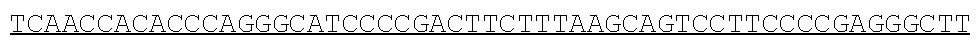

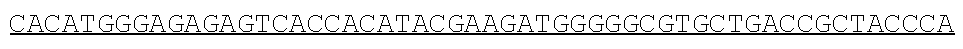

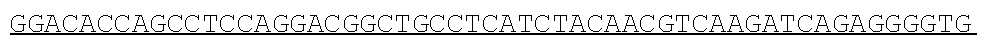

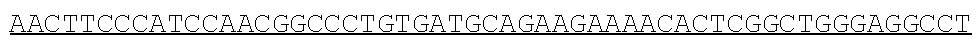

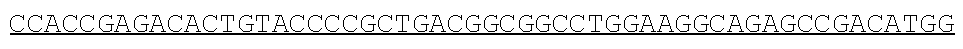

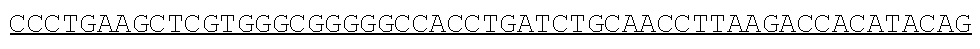

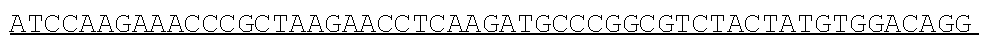

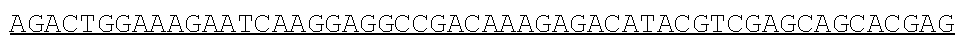

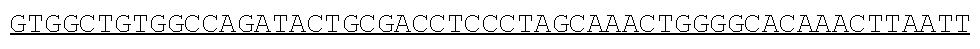

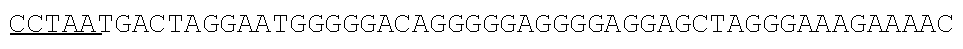

CTGGAGTTTGTGCCAGGGTTTTTGGGATTAAGTTCTTCATTCACTAAGGAAGGAATT
GGGAACACAAAGGGTGGGGGCAGGGGAGTTTGGGGCAACTGGTTGGAGGGAAG
GTGAAGTTCAATGATGCTCTTGATTTTAATCCCACATCATGTATCACTTTTTTCTTAA
ATAAAGAAGCCTGGGACACAGTAGATAGACACACTT
ACTB P2A-mKate knock-in HDR template sequence
Left Homology Arm-Insertion Sequence-Right Homology Arm
(Underlined are the inserted mKate fluorescent protein sequence, the proceeding non-
underlined part is the P2A peptide sequence)
(SEQ ID NO: 552)

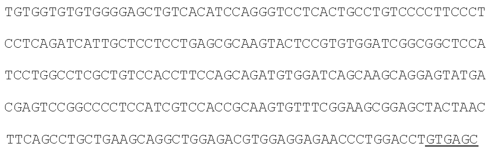

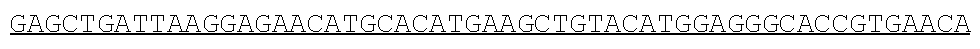

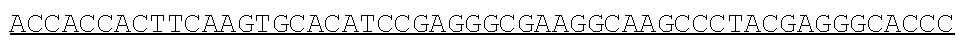

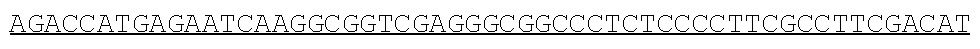

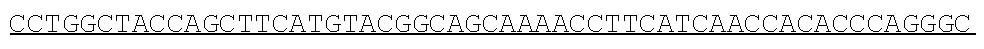

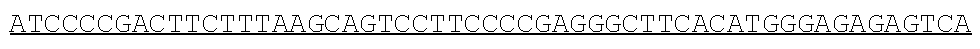

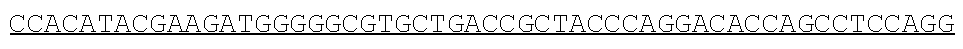

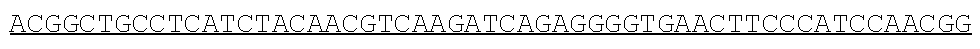

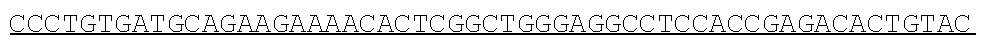

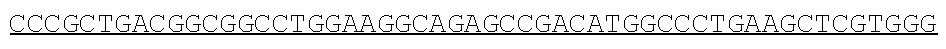

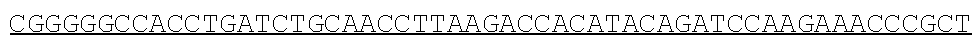

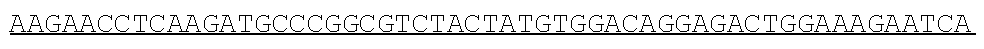

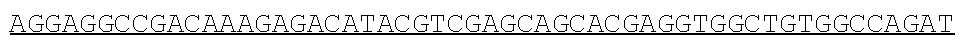

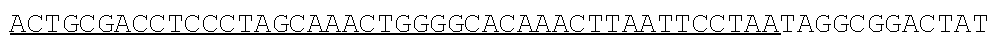

GACTTAGTTGCGTTACACCCTTTCTTGACAAAACCTAACTTGCGCAGGGGGCAAGAT
GAGATTGGCATGGCTTTATTTGTTTTTTTTGTTTTGTTTTGGTTTTTTTTTTTTTTTTG
GCTTGACTCAGGATTTAAAAACTGGAACGGTGAAGGTGACAGCAGTCGGTTGGAG
CGAGCATCCCCCAAAGTTCACAATGTGGCCGAGGACTTTGATTGCACATTGTTGTT
TTTTTAATAGTCATTCCAAATATGAGATGCGTTGTTACAGGAAGTCCCTTGCCATCC
TAAAAGCCACCCCACTTCTCTCTAAGGAGAATGGCCCAGTCCTCTCCCAAGTCCAC
ACAGGGGAGGTGATAGCATTGCTTTCGTGTAA
EMX1 HDR template sequence
Left Homology Arm-Insertion/Replacement Sequence-Right Homology Arm
(Underlined are the inserted BsrGI restriction site, i.e. TGTACA)
(SEQ ID NO: 553)

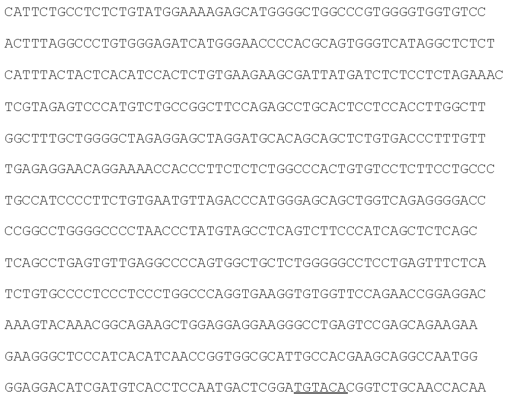

ACCCACGAGGGCAGAGTGCTGCTTGCTGCTGGCCAGGCCCCTGCGTGGGCCCAA
GCTGGACTCTGGCCACTCCCTGGCCAGGCTTTGGGGAGGCCTGGAGTCATGGCC
CCACAGGGCTTGAAGCCCGGGGCCGCCATTGACAGAGGGACAAGCAATGGGCTG
GCTGAGGCCTGGACCACTTGGCCTTCTCCTCGGAGAGCCTGCCTGCCTGGGCG
GGCCCGCCCGCCACCGCAGCCTCCCAGCTGCTCTCCGTGTCTCCAATCTCCCTTT
TGTTTTGATGCATTTCTGTTTTAATTTATTTTCCAGGCACCACTGTAGTTTAGTGATC
CCCAGTGTCCCCCTTCCCTATGGGAATAATAAAAGTCTCTCTCTTAATGACACGGG
CATCCAGCTCCAGCCCCAGAGCCTGGGGTGGTAGATTCCGGCTCTGAGGGCCAG
TGGGGGCTGGTAGAGCAAACGCGTTCAGGGCCTGGGAGCCTGGGGTGGGGTACT
GGTGGAGGGGGTCAAGGGTAATTCATTAACTCCTCTCTTTTGTTGGGGGACCCTG
GTCTCTACCTCCAGCCTCCACAGCAGGAGAAACAGGCTAGACATAGGGAAGGGCCA
TCCTGTATCTTGAGGGAGGACAGGCCCAGGTCTTTCTTAACGTATTGAGAGGTGG
GAATCAGCCCAGGTAGTTCAATGGG
DYNLT1 mKate-T2A-EGFP HDR template
Left Homology Arm-mKate-T2Alinker-EGFP-Right Homology Arm
(Underlined are the inserted mKate/EGFP fluorescent protein sequence, with the
connecting non-underlined T2A peptide sequence)
(SEQ ID NO: 554)

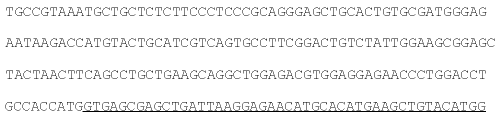

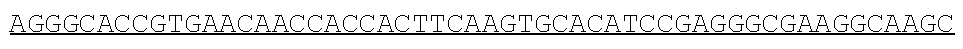

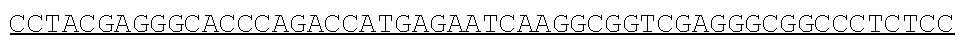

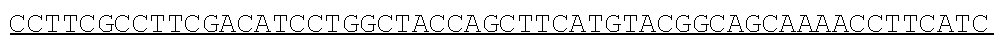

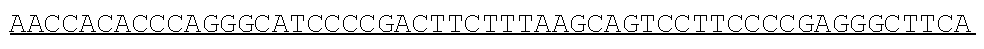

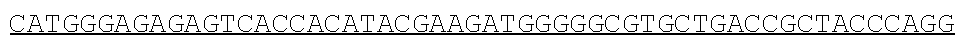

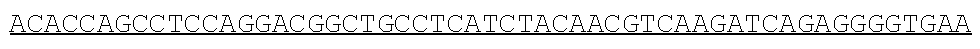

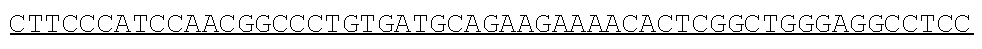

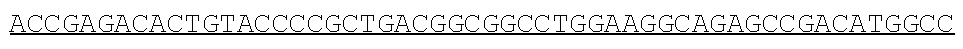

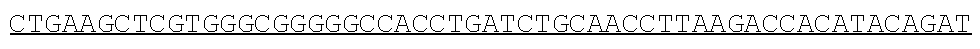

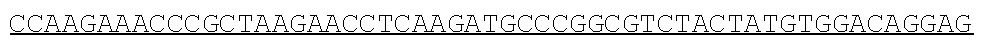

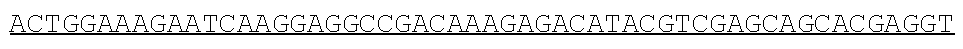

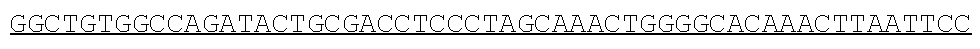

GCTAGCGGCAGTGGAGAGGGCAGAGGAAGTCTGCTAACATGCGGTGACGTCGAG
GAGAATCCTGGCCCAGGTGGTTCTGCCGGTGGCTCCGGTTCTGGCTCCAGCGGT
GGCAGCTCTGGTGCGTCCGGCACGGGTACTGCGGGTGGCACTGGCAGCGGTTCC

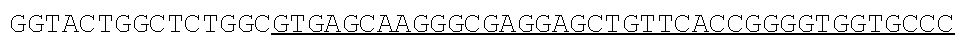

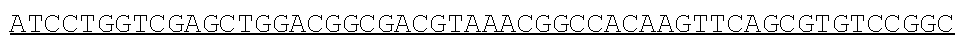

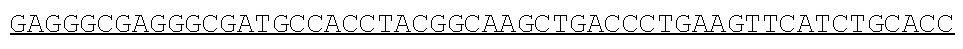

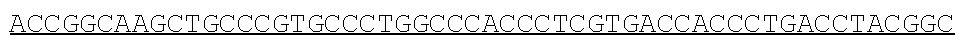

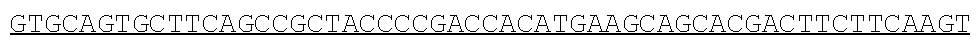

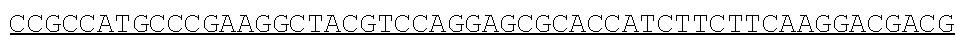

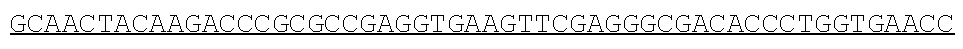

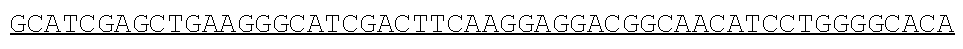

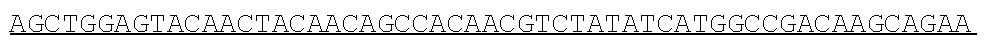

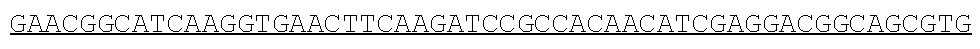

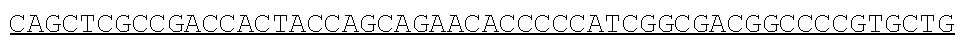

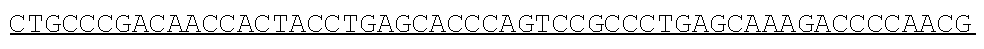

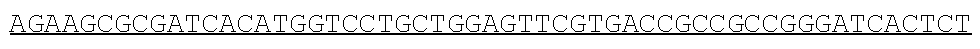

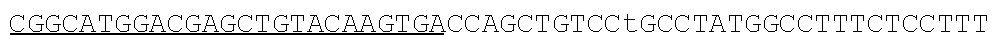

TGTCTCTAGTTCATCCTCTAACCACCAGCCATGAATTCAGTGAACTCTTTTCTCATT
CTCTTTGTTTTGTGGCACTTTCACAATGTAGAGGAAAAAACCAATGACCGCACTGT
GATGTGAATGGCACCGAAGTCAGATGAGTATCCCTGTAGGTCACCTGCAGCCTGC
GTTGCCACTTGTCTT
HSP90AA1 mKate-T2A-EGFP HDR template
Left Homology Arm-mKate-T2Alinker-EGFP-Right Homology Arm
(Underlined are the inserted mKate/EGFP fluorescent protein sequence, with the
connecting non-underlined T2A peptide sequence)
(SEQ ID NO: 555)

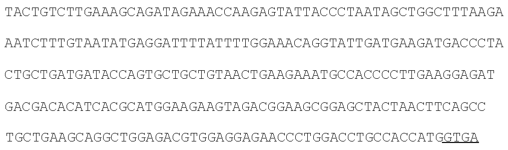

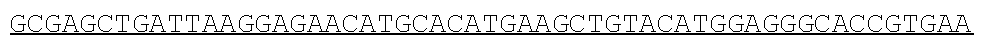

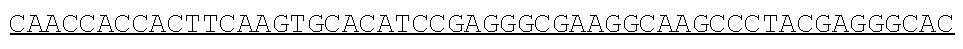

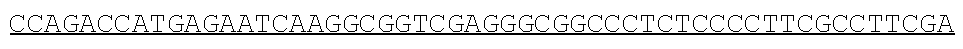

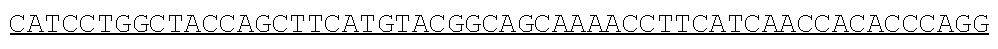

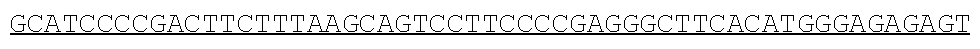

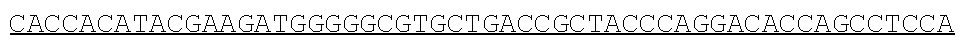

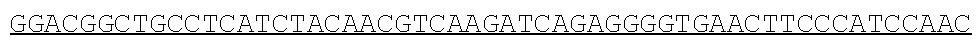

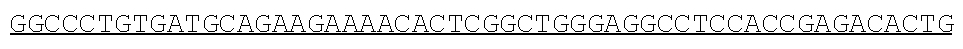

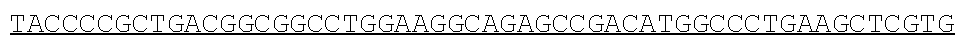

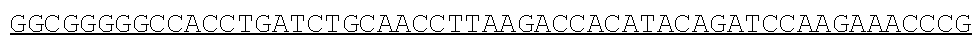

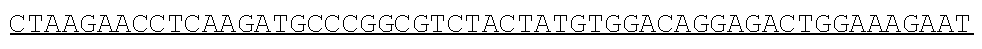

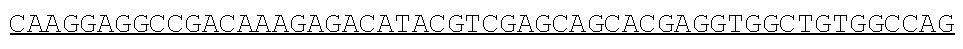

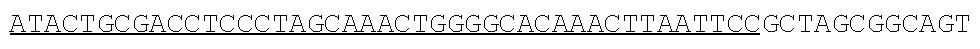

GGAGAAAACAGAGGAAGTCTGCTAACATGCGGTGACGTCGAGGAGAATCCTGGC
CCAGGTGGTTCTGCCGGTGGCTCCGGTTCTGGCTCCAGCGGTGGCAGCTCTGGT
GCGTCCGGCACGGGTACTGCGGGTGGCACTGGCAGCGGTTCCGGTACTGGCTCT

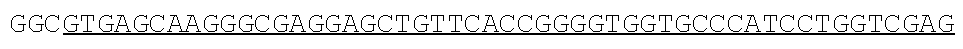

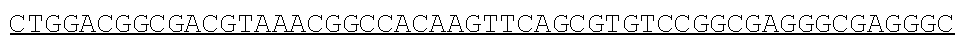

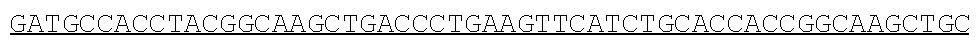

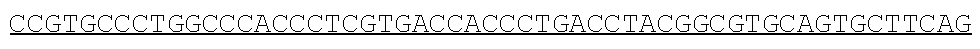

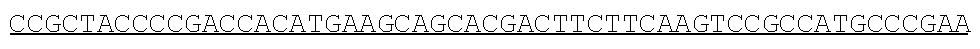

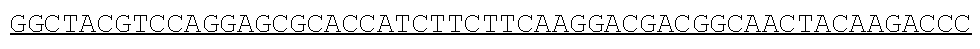

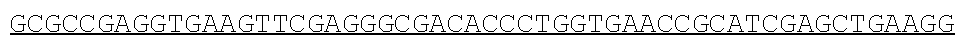

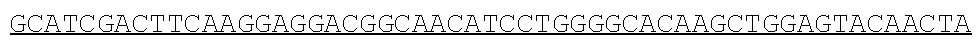

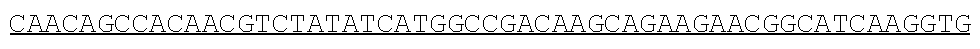

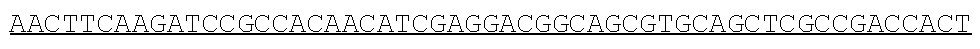

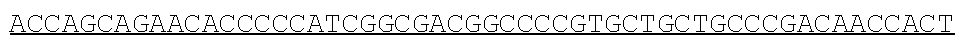

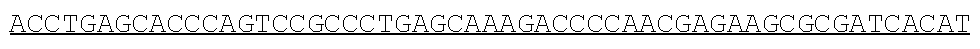

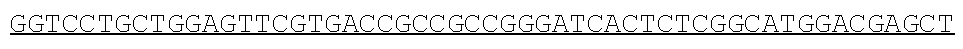

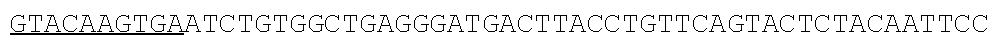

TCTGATAATATATTTTCAAGGATGTTTTTCTTTATTTTTGTTAATATTAAAAAGTCTGT
ATGGCATGACAACTACTTTAAGGGGAAGATAAGATTTCTGTCTACTAAGTGATGCT
GTGATACCTTAGGCACTAAAGCAGAGCTAGTAATGCT
HIST1H2BK P2A-mKate knock-in HDR template sequence
Left Homology Arm-Insertion Sequence-Right Homology Arm
(Underlined are the inserted mKate fluorescent protein sequence, the proceeding non-
uderlined part is the P2A peptide sequence)
(SEQ ID NO: 556)

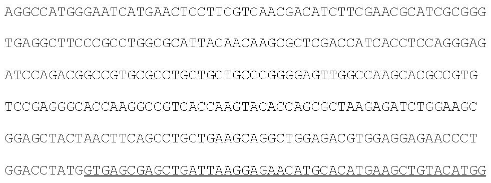

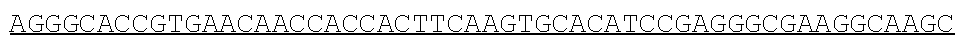

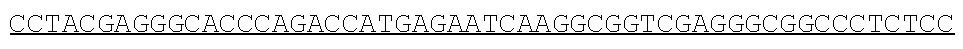

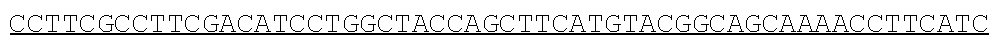

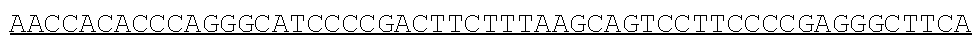

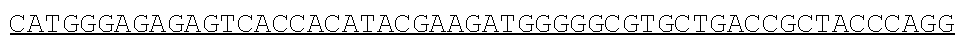

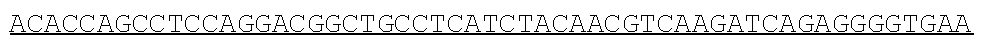

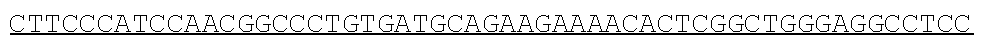

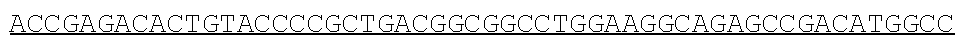

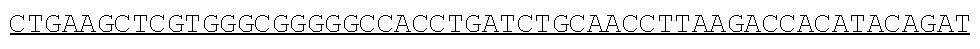

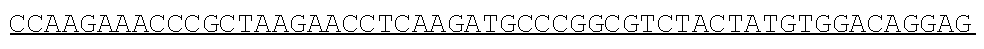

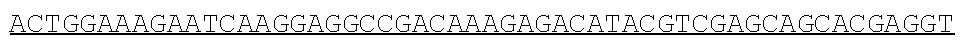

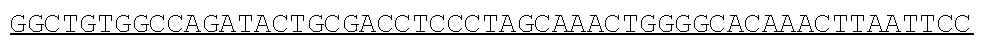

TAATAAACTTGGAAAGTAAGCGTCTTAAAGCCCAACCCCAAAGGCTCTTTTAAGAG
CCACTTAATTATCGATATTAGAGCTGTAAACACGTGTTCCTAATCCAGGCTTAAAT
TCGGGCGATCTTTTTAAAGGGATTATCATGATCTTATCACACTGAGTAATGCATGCA
GTGCTTGTAATCGAGTCTGGCTAAGGAGAAGCCACATACACTTTGTTAGTACTGAG
TGAGGAATATGGCGCCTAAAAAATTAGGGATGTGCAGGAAACTTGTGTTAACAGAA
AGTGCTTCCTGGCTCTTGGCGCGAAAATGGATACTTCCGTTGTGTCGAGTGCTGTG
ACTTCCTGTTAGAACTTGTTGAAAGCCTATTGTGTCACGTGTACTTTCCACCATGTA
ATGGCGTTCTAACGTGAG
BCAP31 P2A-mKate knock-in HDR template sequence
Left Homology Arm-Insertion Sequence-Right Homology Arm
(Underlined are the inserted mKate fluorescent protein sequence, the proceeding non-
underlined part is the P2A peptide sequence)
(SEQ ID NO: 557)

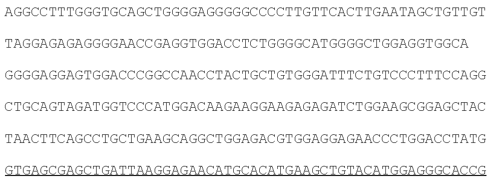

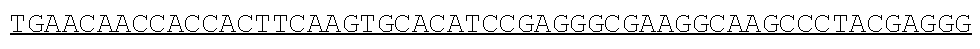

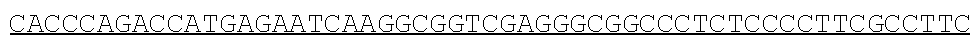

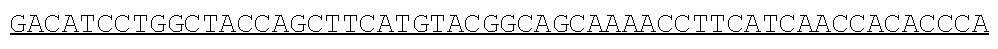

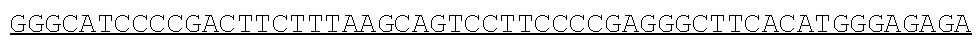

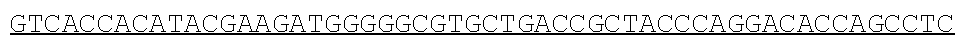

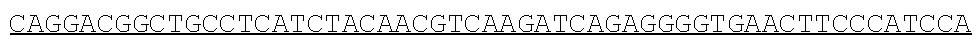

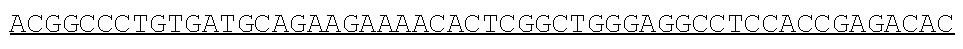

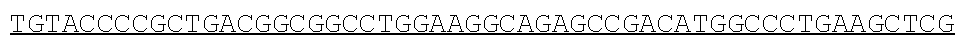

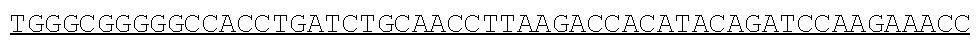

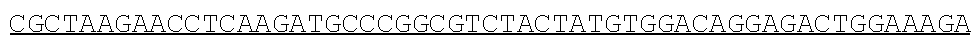

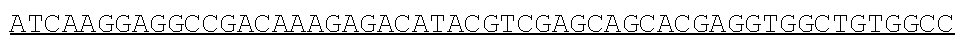

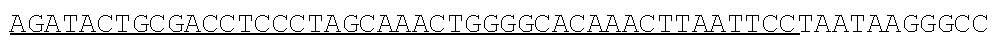

TCCTTCCTCCCCTGCCTGCAGCTGGCTTCCACCTGGCACGTGCCTGCTGCTTCCT
GAGAGCCCGGCCTCTCCCTCCAGTACTTCTGTTTGTGCCCTTCTGCTTCCCCATT
CCCTTCCACAGCTCATAGCTCGTCATCTCGGCCCTTGTCCACACTCTCCAAGCACA
TTACAGGGGACCTGATTGCTACACGTTCAGAATGCGTTTGCTGTCATCCTGCTTGG
CCTGGCCAGGCCTGGCACAGCCTTGGCTTCCACGCCTGAGCGTGGAGAGCACGA
GTTAGTTGTAGTCCGGCTTGCGGTGGGGCTGACTTCCTGTTGGTTTGAGCCCCTTT
TTGTTTTGCCCTCTGGGTGTTTTCTTTGGTCCCGCAGGAGGGTGGGTGGAGCAGG
TGGACTG
CLTA P2A-mKate knock-in HDR template sequence
Left Homology Arm-Insertion Sequence-Right Homology Arm
(Underlined are the inserted mKate fluorescent protein sequence, the proceeding non-
underlined part is the P2A peptide sequence)
(SEQ ID NO: 558)

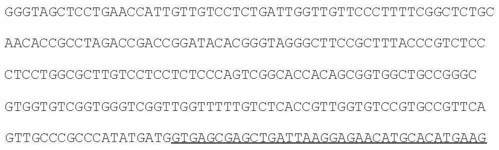

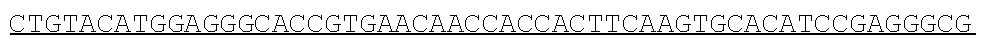

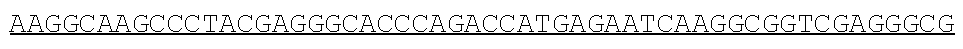

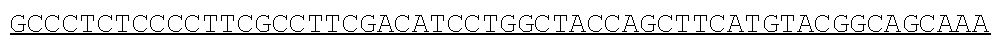

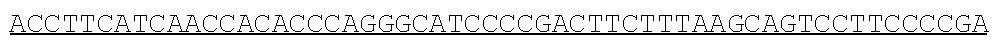

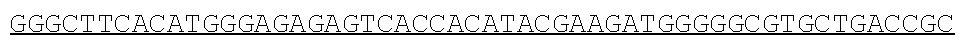

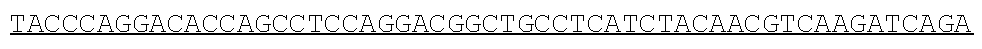

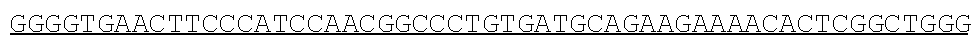

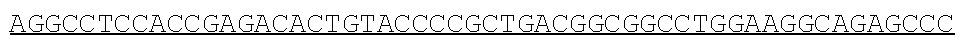

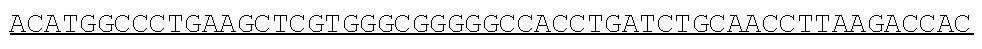

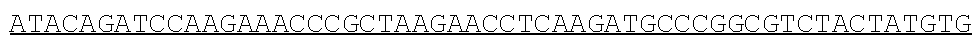

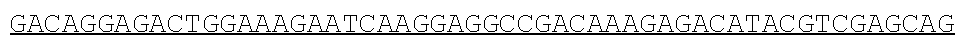

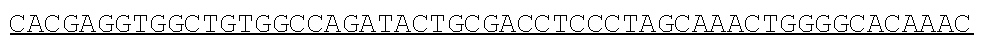

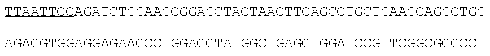

TGCCGGCGCCCCTGGCGGTCCCGCGCTGGGGAACGGAGTGGCCGGCGCCCGGCG
AAGAAGACCCGGCTGCGGCCTTCTTGGCGCAGCAAGAGAGCGAGATTGCGGGCA
TCGAGAACGACGAGGCCTTCGCCATCCTGGACGGCGGCGCCCCCGGGCCCCAG
CCGCACGGCGAGCCGCCGGGGGGTCCGGGTGAGAGTGCGGGCGCGTTTGGGGC
GAGAGGACTTGTCTGGAAACTCGGTCCACAGTGGGTCCGAGAGCTTCTGTGTGAC
TCGTGCTCCTTGCTGAATTAGGAGGTTAGGGAGCAGTGCAAACAGGAAACGAGAC
CCTGGCCCGGTCTTTCAGAAACCTAGGCTCGAGAAGCCTGTTCGGTTCTCAGCAT
GTTTGAGTGCTTCTGG
RAB11A P2A-mKate knock-in HDR template sequence
Left Homology Arm-Insertion Sequence-Right Homology Arm
(Underlined are the inserted mKate fluorescent protein sequence, the proceeding non-
underlined part is the P2A peptide sequence)
(SEQ ID NO: 559)

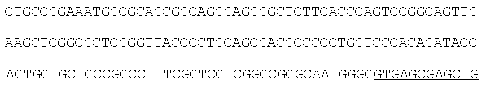

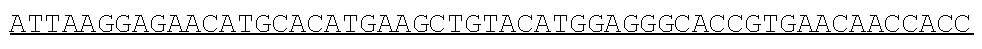

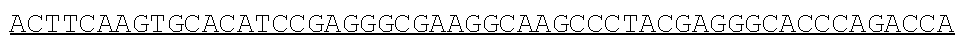

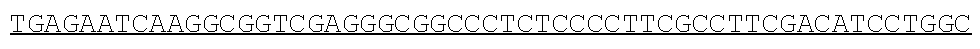

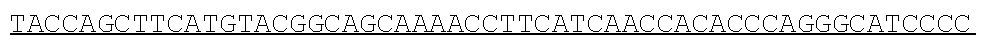

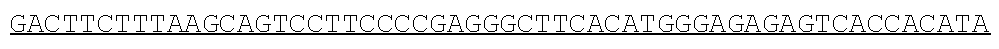

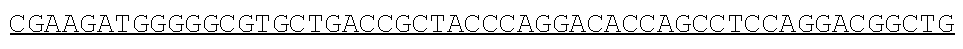

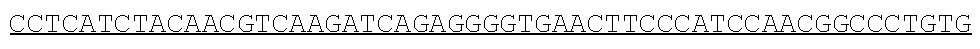

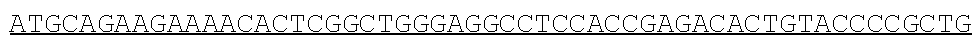

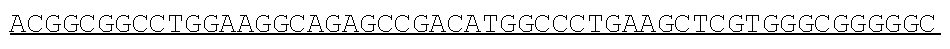

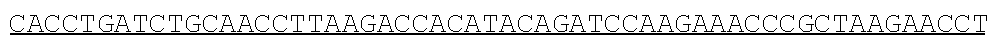

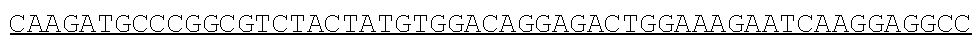

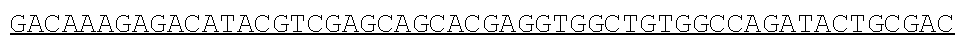

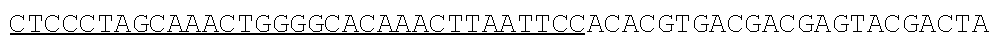

CCTCTTTAAAGGTGAGGCCATGGGCTCTGCACTCTACACAGTCCTCGTTCGGGG
ACCCGGGCCACTCCCGGTGGACCCTCGTGCCGGCCACCCCTGCACTGATATAGG
CCTCCTCAGCCCTTCCTTTTTGTGCGGTTCCGTCTCCTAC

### Example 19

Arrayed SSAP library screening on endogenous genome targets (ACTB. HSP90AA1) using mKate knock-in assay.

SSAP-encoding plasmids were purified and quantified.

Each SSAP encoding plasmid was tested in duplicate, including a negative control (same plasmid encoding Flag_HA which is not expected to promote gene editing). Transfections were in 96-well plates and transfection efficiency was estimated to be 50%.

### Knock-In Templates:

1. HSP90AA1: gCK240+241, tm 66.1C, mKate/pCK1451/pCK1452 as PCR template
2. ACTB: gCK115+116, tm 63.6C, mKate/pCK1453/pCK1454 as PCR templateLG

Three days after transfection, mKate positive cells and cell viability were quantified across all replicates, along with positive (original RecT SSAP) and negative (Flag-HA control protein) controls. Higher frequency of mKate+ cells indicates a candidate SSAP is more active (i.e., has higher ability to mediate precision knock-in editing of the kilobase-scale transgene). At the same time, the cell viability was measured by live cell counts via flow cytometry, to help quantify the fitness effect of SSAP on mammalian cells.

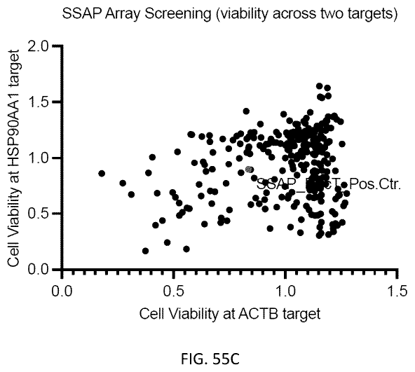

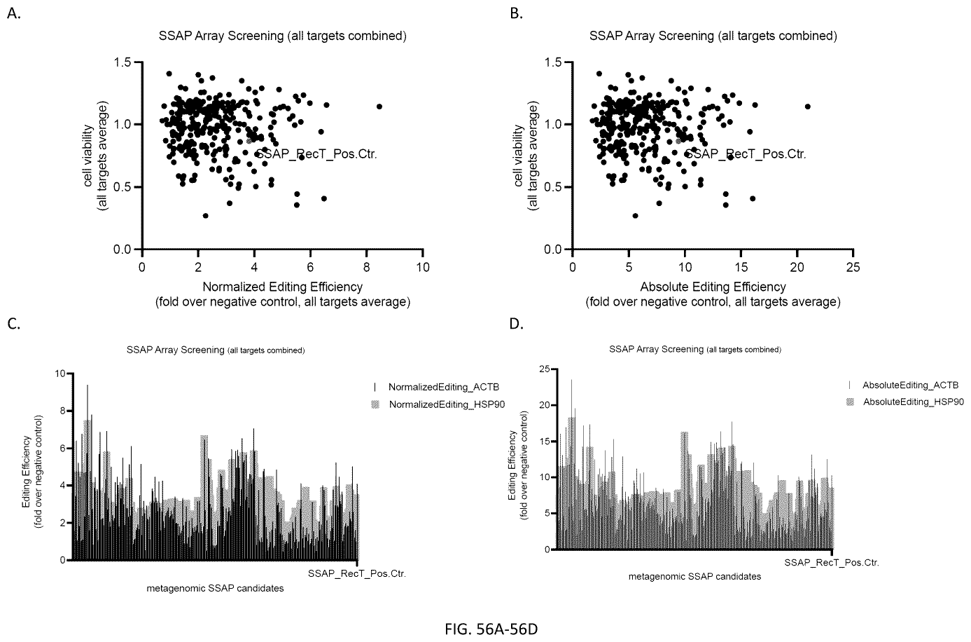

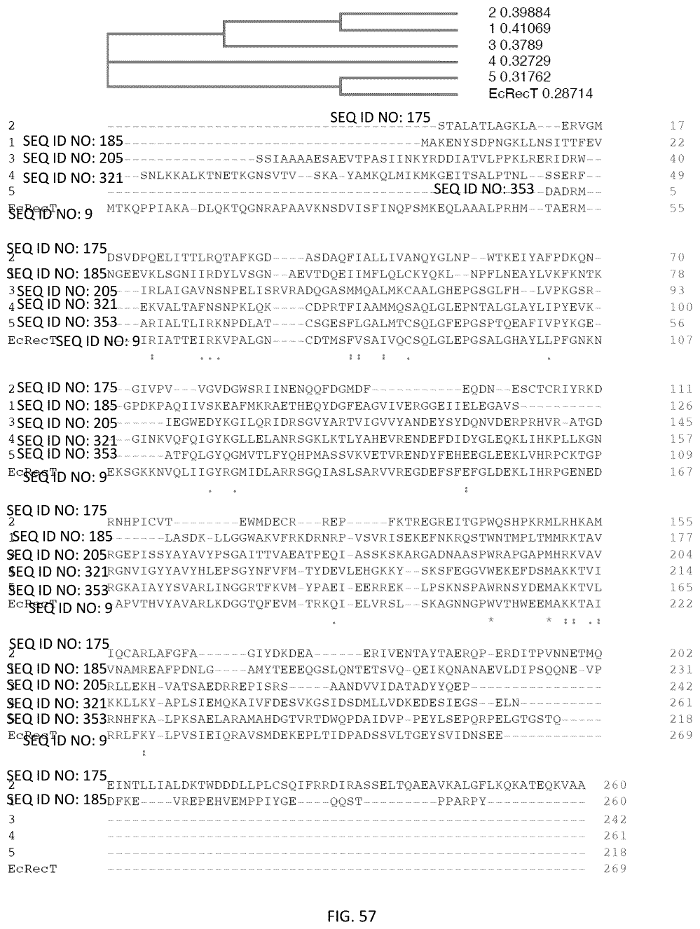

FIG. 55 shows results of SSAP array screening, showing editing efficiency as fold over negative control or percent of mKate knock-in and cell viability for the ACTB target and the HSP90AA1 target. FIG. 56 shows normalized (56A) and absolute (56B) editing efficiency at HSP90AA compared to editing efficiency at ACTB. FIG. 56C shows cell viability, comparing SSAP use for HSP90AA1 knock-ins with ACTB knock-ins. FIG. 57 provides plots comparing cell viability and editing efficiency, normalized (A) and absolute (B) over all targets and (A, B) and bar graphs illustrating normalized (C) or absolute (D) editing efficiency at ACTB and HSP90 for each of the SSAP candidates.

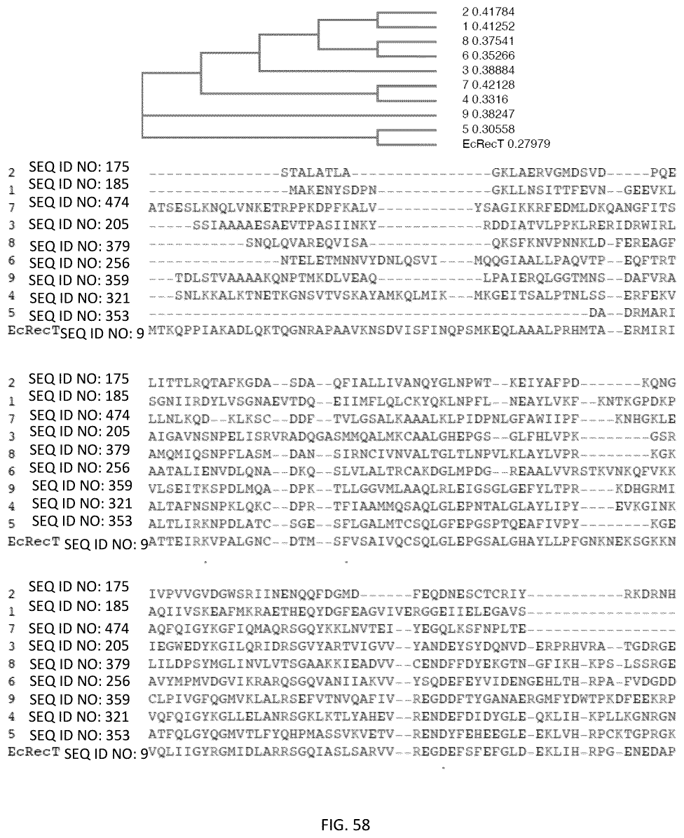

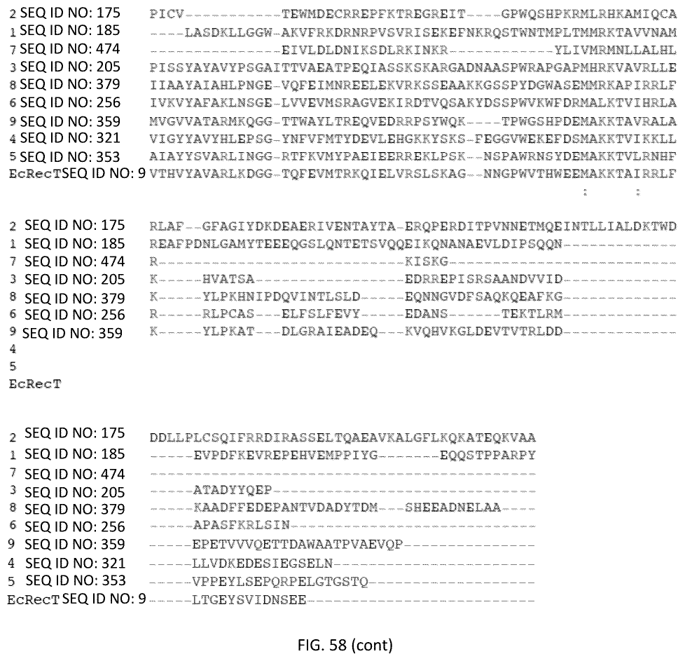

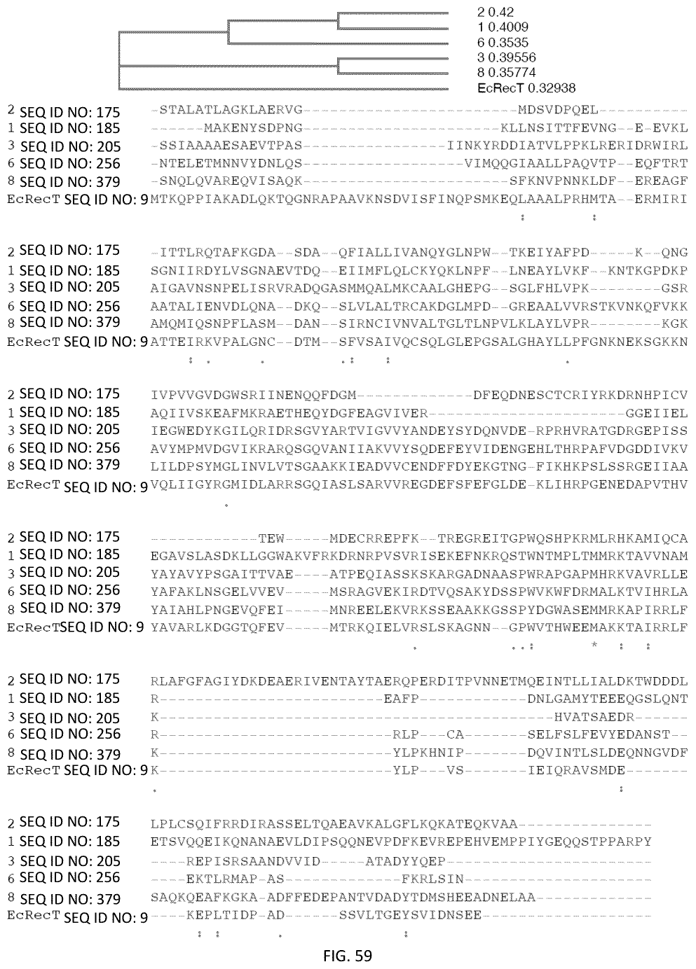

Alignments and phylogenic trees depicting related proteins and sequence alignments for several of the top targets are provided in FIGS. 58, 59, and 60. The alignments indicate certain conserved regions and motifs, consistent with regions of predicted 3D structure (e.g., FIG. 36, 37, 44, 53). At least 3 regions are highly conserved: (1) the N-terminal part has a S/N/Y-R/K-F/L/I-rich region resembling a Serine/Tyrosine recombinase motif; (2) the middle-part has a M-R/K-R/K-rich region; (3) the C-terminal part includes a D/E-D/E-F/Y region that resembles a transposase-like motif. Some candidates SSAP may have one, or more of these regions. This is also in agreement with the predicted 3D structure of SSAP and interaction of the SSAP with DNA that promotes homology-based recombination via highly-charged amino acids.

Top scoring SSAP proteins are shown in Table 9. The table shows editing efficiency as the normalized average of two targets (HSP90 and ACTB), absolute editing efficiency, and cell viability. SSAP proteins are identified by Uniparc deposit number and SEQ ID NO. Alignment numbers correspond to SSAPs in FIGS. 58, 59, and 60.

TABLE 9
Top scoring SSAP proteins

| | | | | Editing | Editing | | |
|---|---|---|---|---|---|---|---|
| | | SSAP | | Efficiency | Efficiency | Cell | |
| | | aa | | (Normalized, | (Absolute, | Viability | |
| | SSAP_protein_id | SEQ | SSAP | Average, | Average, | (Average, | Alignment |
| SSAP_ID | uniparc | ID NO: | Length | two targets) | two targets) | two targets) | No. |
| SSAP_4 | UPI0000030D3E | 166 | 260 | 5.570396093 | 13.8 | 1.192302514 | |
| SSAP_6 | UPI000009B019 | 175 | 260 | 4.879114721 | 12.075 | 1.281594986 | |
| SSAP_10 | UPI000019AB49 | 179 | 260 | 5.750036812 | 14.25 | 1.236640684 | 2 |
| SSAP_12 | UPI00005F0A78 | 181 | 260 | 4.26258344 | 10.5525 | 1.291292719 | |
| SSAP_15 | UPI00015968D7 | 184 | 247 | 5.476976375 | 13.55 | 1.226370883 | |
| SSAP_16 | UPI00015C01AE | 185 | 260 | 8.460844704 | 20.95 | 1.14576164 | 1 |
| SSAP_20 | UPI0001CE597A | 189 | 267 | 5.982113642 | 14.85 | 1.173569699 | |
| SSAP_29 | UPI00025CAD2E | 198 | 260 | 5.204384264 | 12.9225 | 1.130604022 | |
| SSAP_36 | UPI0002E4C0BF | 205 | 242 | 6.38315582 | 15.8 | 0.94183346 | 3 |
| SSAP_67 | UPI000865F43D | 236 | 264 | 4.616392216 | 11.4625 | 1.087954602 | |
| SSAP_152 | WP_147540090.1 | 321 | 261 | 6.57396273 | 16.25 | 1.158741271 | 4 |
| SSAP_158 | WP_051264703.1 | 327 | 272 | 5.439714833 | 13.45 | 0.994538899 | |
| SSAP_172 | WP_145458209.1 | 341 | 265 | 4.239056723 | 10.4925 | 0.904622423 | |
| SSAP_173 | WP_117787252.1 | 342 | 265 | 4.673521809 | 11.55 | 0.878738037 | |
| SSAP_184 | WP_060905391.1 | 353 | 218 | 5.689215831 | 14.075 | 0.736074079 | 5 |
| SSAP_190 | EIC09117.1 | 359 | 268 | 5.259951409 | 13.025 | 1.044246213 | 9 |
| SSAP_191 | WP_136046271.1 | 354 | 272 | 4.749640064 | 11.75 | 1.128706938 | |
| SSAP_192 | WP_136309287.1 | 361 | 272 | 5.371858742 | 13.3 | 1.071092146 | |
| SSAP_193 | WP_110990907.1 | 362 | 256 | 5.034165276 | 12.465 | 1.146734355 | |
| SSAP_194 | WP_109196224.1 | 363 | 270 | 4.924086254 | 12.175 | 1.138604798 | |
| SSAP_197 | PAV10712.1 | 366 | 247 | 5.661014037 | 14.025 | 1.020528991 | 6 |
| SSAP_198 | WP_147981944.1 | 367 | 250 | 5.524340663 | 13.65 | 0.444354117 | |
| SSAP_199 | BAQ93806.1 | 368 | 266 | 4.601904797 | 11.4025 | 0.518628486 | |
| SSAP_210 | WP_123127078.1 | 379 | 265 | 6.483753804 | 16.05 | 0.407958768 | 8 |
| SSAP_211 | WP_093587584.1 | 380 | 272 | 5.516630673 | 13.65 | 0.356287241 | |
| SSAP_220 | WP_116200709.1 | 389 | 268 | 4.598610157 | 11.375 | 0.915229877 | |
| SSAP_221 | WP_020135111.1 | 390 | 270 | 4.586503223 | 11.35 | 0.977166931 | |
| SSAP_235 | WP_148001988.1 | 404 | 264 | 4.251235234 | 10.53 | 0.884712362 | |
| SSAP_262 | ERL63827.1 | 431 | 260 | 3.675765273 | 9.1025 | 1.121804954 | |
| SSAP_288 | WP_080022455.1 | 457 | 261 | 3.8322433 | 9.4725 | 0.683553486 | |
| SSAP_305 | WP_051624047.1 | 424 | 179 | 4.612455417 | 11.425 | 0.557802727 | 7 |
| SSAP_309 | WP_076079849.1 | 478 | 252 | 3.991676483 | 9.885 | 0.922352431 | |
| SSAP_229 | WP_073846185.1 | 398 | 269 | 3.971178545 | 9.8525 | 0.877647599 | |
| SSAP_Pos. | EcRecT | 171 | 269 | 3.807178675 | 9.4225 | 0.865096117 | EcRecT |
| Control_325 | | | | | | | |

### REFERENCES

1. D. Carroll, Genome engineering with targetable nucleases. Annu. Rev. Biochem. 83, 409-439 (2014).
2. A. Pickar-Oliver, C. A. Gersbach, The next generation of CRISPR-Cas technologies and applications. Nat. Rev. Mol. Cell Biol. 20, 490-507 (2019).
3. R. Barrangou, J. A. Doudna, Applications of CRISPR technologies in research and beyond. Nat. Biotechnol. 34, 933-941 (2016).
4. P. D. Hsu, E. S. Lander, F. Zhang, Development and applications of CRISPR-Cas9 for genome engineering. Cell. 157, 1262-1278 (2014).
5. J. A. Doudna, E. Charpentier, The new frontier of genome engineering with CRISPR-Cas9. Science. 346, 1258096 (2014).
6. J. D. Sander, J. K. Joung, CRISPR-Cas systems for editing, regulating and targeting genomes. Nat. Biotechnol. 32, 347-355 (2014).
7. T. Gaj, C. A. Gersbach, C. F. Barbas, ZFN, TALEN, and CRISPR/Cas-based methods for genome engineering. Trends Biotechnol. 31, 397-405 (2013).
8. F. D. Urnov, E. J. Rebar, M. C. Holmes, H. S. Zhang, P. D. Gregory, Genome editing with engineered zinc finger nucleases. Nat. Rev. Genet. 11, 636-646 (2010).
9. H. Kim, J.-S. Kim, A guide to genome engineering with programmable nucleases. Nat. Rev. Genet. 15, 321-334 (2014).
10. W. Jiang, L. A. Marraffini, CRISPR-Cas: New Tools for Genetic Manipulations from Bacterial Immunity Systems. Annu. Rev. Microbiol. 69, 209-228 (2015).
11. A. V. Anzalone, L. W. Koblan, D. R. Liu, Genome editing with CRISPR-Cas nucleases, base editors, transposases and prime editors. Nat. Biotechnol. 38, 824-844 (2020).
12. M. Jasin, J. E. Haber, The democratization of gene editing: Insights from site-specific cleavage and double-strand break repair. DNA Repair. 44, 6-16 (2016).
13. N. Maizels, L. Davis, Initiation of homologous recombination at DNA nicks. Nucleic Acids Res. 46, 6962-6973 (2018).
14. S. Q. Tsai, J. K. Joung, Defining and improving the genome-wide specificities of CRISPR-Cas9 nucleases. Nat. Rev. Genet. 17, 300-312 (2016).
15. D. Kim, K. Luk, S. A. Wolfe, J.-S. Kim, Evaluating and Enhancing Target Specificity of Gene-Editing Nucleases and Deaminases. Annu. Rev. Biochem. 88, 191-220 (2019).
16. R. J. Ihry, K. A. Worringer, M. R. Salick, E. Frias, D. Ho, K. Theriault, S. Kommineni, J. Chen, M. Sondey, C. Ye, R. Randhawa, T. Kulkarni, Z. Yang, G. McAllister, C. Russ, J. Recce-Hoyes, W. Forrester, G. R. Hoffman, R. Dolmetsch, A. Kaykas, p53 inhibits CRISPR-Cas9 engineering in human pluripotent stem cells. Nat. Med. 24, 939-946 (2018).
17. O. M. Enache, V. Rendo, M. Abdusamad, D. Lam, D. Davison, S. Pal, N. Currimjec, J. Hess, S. Pantel, A. Nag, A. R. Thorner, J. G. Doench, F. Vazquez, R. Beroukhim, T. R. Golub, U. Ben-David, Cas9 activates the p53 pathway and selects for p53-inactivating mutations. Nat. Genet. 52, 662-668 (2020).
18. E. Haapanicmi, S. Botla, J. Persson, B. Schmierer, J. Taipale, CRISPR-Cas9 genomc editing induces a p53-mediated DNA damage response. Nat. Med. 24, 927-930 (2018).
19. C. E. Dunbar, K. A. High, J. K. Joung, D. B. Kohn, K. Ozawa, M. Sadelain, Gene therapy comes of age. Science. 359, caan4672 (2018).
20. F. Mingozzi, K. A. High, Therapeutic in vivo gene transfer for genetic disease using AAV: progress and challenges. Nat. Rev. Genet. 12, 341-355 (2011).
21. D. Wang, F. Zhang, G. Gao, CRISPR-Based Therapeutic Genome Editing: Strategies and In Vivo Delivery by AAV Vectors. Cell. 181, 136-150 (2020).
22. H. Shivram, B. F. Cress, G. J. Knott, J. A. Doudna, Controlling and enhancing CRISPR systems. Nat. Chem. Biol. 17, 10-19 (2021).
23. C. D. Yeh, C. D. Richardson, J. E. Corn, Advances in genome editing through control of DNA repair pathways. Nat. Cell Biol. 21, 1468-1478 (2019).
24. K. S. Pawelczak, N. S. Gavande, P. S. VanderVere-Carozza, J. J. Turchi, Modulating DNA Repair Pathways to Improve Precision Genome Engineering. ACS Chem. Biol. 13, 389-396 (2018).
25. N. G. Copeland, N. A. Jenkins, D. L. Court, Recombineering: a powerful new tool for mouse functional genomics. Nat. Rev. Genet. 2, 769-779 (2001).
26. R. Kolodner, S. D. Hall, C. Luisi-DeLuca, Homologous pairing proteins encoded by the Escherichia coli recE and recT genes. Mol. Microbiol. 11, 23-30 (1994).
27. L. M. Iyer, E. V. Koonin, L. Aravind, Classification and evolutionary history of the single-strand annealing proteins, RecT, Redbeta, ERF and RAD52. BMC Genomics. 3, 8 (2002).
28. D. L. Court, J. A. Sawitzke, L. C. Thomason, Genetic Engineering Using Homologous Rccombination. Annu. Rev. Genet. 36, 361-388 (2002).
29. Y. Zhang, F. Buchholz, J. P. P. Muyrers, A. F. Stewart, A new logic for DNA engineering using recombination in Escherichia coli. Nat. Genet. 20, 123-128 (1998).
30. S. Datta, N. Costantino, X. Zhou, D. L. Court, Identification and analysis of recombineering functions from Gram-negative and Gram-positive bacteria and their phages. Proc. Natl. Acad. Sci. U.S.A 105, 1626-1631 (2008).
31. C. Wang, J. K. W. Cheng, Q. Zhang, N. W. Hughes, Q. Xia, M. M. Winslow, L. Cong, Microbial single-strand annealing proteins enable CRISPR gene-editing tools with improved knock-in efficiencies and reduced off-target effects. Nucleic Acids Res. 49, c36-c36 (2021).
32. M. Jinek, K. Chylinski, I. Fonfara, M. Hauer, J. A. Doudna, E. Charpentier, A programmable dual-RNA-guided DNA endonuclease in adaptive bacterial immunity. Science. 337, 816-821 (2012).
33. L. S. Qi, M. H. Larson, L. A. Gilbert, J. A. Doudna, J. S. Weissman, A. P. Arkin, W. A. Lim, Repurposing CRISPR as an RNA-guided platform for sequence-specific control of gene expression. Cell. 152, 1173-1183 (2013).
34. D. Bikard, W. Jiang, P. Samai, A. Hochschild, F. Zhang, L. A. Marraffini, Programmable repression and activation of bacterial gene expression using an engineered CRISPR-Cas system. Nucleic Acids Res. 41, 7429-7437 (2013).
35. M. L. Macder, S. J. Linder, V. M. Cascio, Y. Fu, Q. H. Ho, J. K. Joung, CRISPR RNA-guided activation of endogenous human genes. Nat. Methods. 10, 977-979 (2013).
36. L. A. Gilbert, M. H. Larson, L. Morsut, Z. Liu, G. A. Brar, S. E. Torres, N. Stern-Ginossar, O. Brandman, E. H. Whitehead, J. A. Doudna, W. A. Lim, J. S. Weissman, L. S. Qi, CRISPR-mediated modular RNA-guided regulation of transcription in eukaryotes. Cell. 154, 442-451 (2013).
37. P. Perez-Pincra, D. D. Kocak, C. M. Vockley, A. F. Adler, A. M. Kabadi, L. R. Polstein, P. I. Thakore, K. A. Glass, D. G. Ousterout, K. W. Leong, F. Guilak, G. E. Crawford, T. E. Reddy, C. A. Gersbach, RNA-guided gene activation by CRISPR-Cas9-based transcription factors. Nat. Methods. 10, 973-976 (2013).
38. F. Farzadfard, S. D. Perli, T. K. Lu, Tunable and multifunctional eukaryotic transcription factors based on CRISPR/Cas. ACS Synth. Biol. 2, 604-613 (2013).
39. D. L. Jones, P. Leroy, C. Unoson, D. Fange, V. Curic, M. J. Lawson, J. Elf, Kinetics of dCas9 target search in Escherichia coli. Science. 357, 1420-1424 (2017).
40. S. H. Sternberg, S. Redding, M. Jinek, E. C. Greene, J. A. Doudna, DNA interrogation by the CRISPR RNA-guided endonuclease Cas9. Nature. 507, 62-67 (2014).
41. S. C. Knight, L. Xie, W. Deng, B. Guglielmi, L. B. Witkowsky, L. Bosanac, E. T. Zhang, M. El Beheiry, J.-B. Masson, M. Dahan, Z. Liu, J. A. Doudna, R. Tjian, Dynamics of CRISPR-Cas9 genome interrogation in living cells. Science. 350, 823-826 (2015).
42. P. A. Carr, G. M. Church, Genome engineering. Nat. Biotechnol. 27, 1151-1162 (2009).
43. K. M. Esvelt, H. H. Wang, Genome-scale engineering for systems and synthetic biology. Mol. Syst. Biol. 9, 641 (2013).
44. N. Rybalchenko, E. I. Golub, B. Bi, C. M. Radding, Strand invasion promoted by recombination protein beta of coliphage lambda. Proc. Natl. Acad. Sci. U.S.A 101, 17056-17060 (2004).
45. J. A. Mosberg, M. J. Lajoie, G. M. Church, Lambda red recombineering in Escherichia coli occurs through a fully single-stranded intermediate. Genetics. 186, 791-799 (2010).
46. J. P. Muyrers, Y. Zhang, F. Buchholz, A. F. Stewart, RecE/RecT and Redalpha/Redbeta initiate double-stranded break repair by specifically interacting with their respective partners. Genes Dev. 14, 1971-1982 (2000).
47. T. M. Wannier, A. Nyerges, H. M. Kuchwara, M. Czikkely, D. Balogh, G. T. Filsinger, N. C. Borders, C. J. Gregg, M. J. Lajoie, X. Rios, C. Pál, G. M. Church, Improved bacterial recombineering by parallelized protein discovery. Proc. Natl. Acad. Sci. U.S.A 117, 13689-13698 (2020).
48. P. Noirot, R. D. Kolodner, DNA strand invasion promoted by Escherichia coli RecT protein. J. Biol. Chem. 273, 12274-12280 (1998).
49. L. C. Thomason, N. Costantino, D. L. Court, Examining a DNA Replication Requirement for Bacteriophage λ Red- and Rac Prophage RecET-Promoted Recombination in Escherichia coli. mBio. 7 (2016), doi:10.1128/mBio.01443-16.
50. P. Baumann, F. E. Benson, S. C. West, Human Rad51 protein promotes ATP-dependent homologous pairing and strand transfer reactions in vitro. Cell. 87, 757-766 (1996).
51. T. Sakuma, S. Nakade, Y. Sakanc, K.-I. T. Suzuki, T. Yamamoto, MMEJ-assisted gene knock-in using TALENs and CRISPR-Cas9 with the PITCh systems. Nat. Protoc. 11, 118-133 (2016).
52. M. Charpentier, A. H. Y. Khedher, S. Menoret, A. Brion, K. Lamribet, E. Dardillac, C. Boix, L. Perrouault, L. Tesson, S. Geny, A. D. Cian, J. M. Itier, I. Anegon, B. Lopez, C. Giovannangeli, J. P. Concordet, CtIP fusion to Cas9 enhances transgene integration by homology-dependent repair. Nat. Commun. 9, 1-11 (2018).
53. T. Gutschner, M. Haemmerle, G. Genovese, G. F. Dractta, L. Chin, Post-translational Regulation of Cas9 during G1 Enhances Homology-Directed Repair. Cell Rep. 14, 1555-1566 (2016).
54. H. A. Rees, W.-H. Ych, D. R. Liu, Development of hRad51-Cas9 nickase fusions that mediate HDR without double-stranded breaks. Nat. Commun. 10, 1-12 (2019).
55. Z. Zhu, N. Verma, F. González, Z.-D. Shi, D. Huangfu, A CRISPR/Cas-Mediated Selection-free Knockin Strategy in Human Embryonic Stem Cells. Stem Cell Rep. 4, 1103-1111 (2015).
56. A. Dupré, L. Boyer-Chatenet, R. M. Sattler, A. P. Modi, J.-H. Lee, M. L. Nicolette, L. Kopelovich, M. Jasin, R. Baer, T. T. Paull, J. Gautier, A forward chemical genetic screen reveals an inhibitor of the Mre11-Rad50-Nbs1 complex. Nat. Chem. Biol. 4, 119-125 (2008).
57. B. Budkc, H. L. Logan, J. H. Kalin, A. S. Zelivianskaia, W. Cameron McGuire, L. L. Miller, J. M. Stark, A. P. Kozikowski, D. K. Bishop, P. P. Connell, RI-1: a chemical inhibitor of RAD51 that disrupts homologous recombination in human cells. Nucleic Acids Res. 40, 7347-7357 (2012).
58. F. Huang, N. A. Motlekar, C. M. Burgwin, A. D. Napper, S. L. Diamond, A. V. Mazin, Identification of specific inhibitors of human RAD51 recombinase using high-throughput screening. ACS Chem. Biol. 6, 628-635 (2011).
59. N. Hustedt, D. Durocher, The control of DNA repair by the cell cycle. Nat. Cell Biol. 19, 1-9 (2016).
60. C. J. Bostock, D. M. Prescott, J. B. Kirkpatrick, An evaluation of the double thymidine block for synchronizing mammalian cells at the G1-S border. Exp. Cell Res. 68, 163-168 (1971).
61. K. Shedden, S. Cooper, Analysis of cell-cycle-specific gene expression in human cells as determined by microarrays and double-thymidine block synchronization. Proc. Natl. Acad. Sci. U.S.A 99, 4379-4384 (2002).
62. F. A. Ran, L. Cong, W. X. Yan, D. A. Scott, J. S. Gootenberg, A. J. Kriz, B. Zetsche, O. Shalem, X. Wu, K. S. Makarova, E. V. Koonin, P. A. Sharp, F. Zhang, In vivo genome editing using Staphylococcus aureus Cas9. Nature. 520, 186-191 (2015).
63. R. J. Austin, T. Xia, J. Ren, T. T. Takahashi, R. W. Roberts, Designed arginine-rich RNA-binding peptides with picomolar affinity. J. Am. Chem. Soc. 124, 10966-10967 (2002).
64. S. Q. Tsai et al., GUIDE-seq enables genome-wide profiling of off-target cleavage by CRISPR-Cas nucleases. Nat Biotechnol 33, 187-197 (2015).
65. C. L. Nobles et al., iGUIDE: an improved pipeline for analyzing CRISPR cleavage specificity. Genome Biol 20, 14 (2019).
66. S. Nakade et al., Microhomology-mediated end-joining-dependent integration of donor DNA in cells and animals using TALENs and CRISPR/Cas9. Nat Commun 5, 5560 (2014).
67 A. Paix et al., Precision genome editing using synthesis-dependent repair of Cas9-induced DNA breaks. Proc Natl Acad Sci USA 114, E10745-E10754 (2017).
68. O. Kanca et al., An efficient CRISPR-based strategy to insert small and large fragments of DNA using short homology arms. Elife 8, (2019).
69. K. J. Tatiossian et al., Rational Selection of CRISPR-Cas9 Guide RNAs for Homology-Directed Genome Editing. Mol Ther 29, 1057-1069 (2021).
70. C. Wang et al., dCas9-based gene editing for cleavage-free genomic knock-in of long sequences. Nat. Cell. Biol. 24, 268-278 (2022).

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A system comprising:
a Cas protein;
a nucleic acid molecule comprising a guide RNA sequence that is complementary to a target DNA sequence; and
a recombination protein,
wherein the recombination protein comprises an exonuclease, a single stranded DNA annealing protein (SSAP), or a single stranded DNA binding protein (SSB), or a combination of two or more thereof.

2. The system of claim 1, wherein the recombination protein comprises an amino acid sequence having at least 95% identity to a recombination protein of SEQ ID NO: 166 to SEQ ID NO: 491.

3. The system of claim 1, wherein the recombination protein comprises an amino acid sequence having at least 95% identity to a recombination protein of Table 9.

4. The system of claim 1, wherein the recombination protein comprises an amino acid sequence having at least 95% identity to SEQ ID NO:179, SEQ ID NO: 185, SEQ ID NO:205, SEQ ID NO:321, SEQ ID NO:353, SEQ ID NO:359, SEQ ID NO: 366, SEQ ID NO:424, SEQ ID NO:474, or SEQ ID NO:479.

5. The system of claim 1, wherein the recombination protein comprises an amino acid sequence having at least 95% identity to SEQ ID NO: 166, SEQ ID NO: 168, SEQ ID NO: 169, SEQ ID NO:170, SEQ ID NO:171, SEQ ID NO:241, SEQ ID NO:253, SEQ ID NO: 290, SEQ ID NO:408, SEQ ID NO:411, or SEQ ID NO:442.

6. The system of claim 1, further comprising a recruitment system comprising.
at least one aptamer sequence; and
an aptamer binding protein functionally linked to the microbial recombination protein as part of a fusion protein.

7. The system of claim 6, wherein the at least one aptamer sequence is an RNA aptamer sequence or a peptide aptamer sequence.

8. The system of claim 7, wherein the nucleic acid molecule comprises the at least one RNA aptamer sequence.

9. The system of claim 8, wherein the nucleic acid molecule comprises two RNA aptamer sequences.

10. The system of claim 9, wherein the two RNA aptamer sequences comprise the same sequence.

11. The system of claim 6, wherein the aptamer binding protein comprises a MS2 coat protein, or a functional derivative or variant thereof.

12. The system of claim 6, wherein the aptamer binding protein comprises phage N peptide, or a functional derivative or variant thereof.

13. The system of claim 7, wherein the at least one peptide aptamer sequence is conjugated to the Cas protein.

14. The system of claim 13, wherein the at least one peptide aptamer sequence comprises between 1 and 24 peptide aptamer sequences.

15. The system of claim 13, wherein the aptamer sequences comprise the same sequence.

16. The system of claim 6, wherein the aptamer sequence comprises a GCN4 peptide sequence.

17. The system of claim 6, wherein the recombination protein N-terminus is linked to the aptamer binding protein C-terminus.

18. The system of claim 6, wherein the fusion protein further comprises a linker between the microbial recombination protein and the aptamer binding protein.

19. The system of claim 18, wherein the linker comprises the amino acid sequence of SEQ ID NO:15.

20. The system of claim 6, wherein the fusion protein further comprises a nuclear localization sequence.

21. The system of claim 20, wherein the nuclear localization sequence comprises the amino acid sequence of SEQ ID NO:16.

22. The system of claim 20, wherein the nuclear localization sequence is on the recombination protein C-terminus.

23. The system of claim 1, wherein the Cas protein is catalytically dead.

24. The system of claim 1, wherein the Cas protein comprises Cas9 or Cas12a.

25. The system of claim 1, wherein the Cas9 protein comprises wild-type Streptococcus pyogenes Cas9 or a wild type Staphylococcus aureus Cas9.

26. The system of claim 1, wherein the Cas protein comprises a nickase.

27. The system of claim 26, wherein the nickase comprises wild-type Streptococcus pyogenes Cas9 with an amino acid substation at position 10 of D10A.

28. The system of claim 1, further comprising donor nucleic acid.

29. The system of claim 1, wherein the target DNA sequence is a genomic DNA sequence in a host cell.

30. A composition comprising:
a polynucleotide comprising a nucleic acid sequence encoding a fusion protein comprising a recombination protein functionally linked to an aptamer binding protein, wherein the recombination protein comprises an amino acid sequence having at least 95% identity to a recombination protein of SEQ ID NO:166 to SEQ ID NO:491;
a polynucleotide comprising a nucleic acid sequence encoding a Cas protein; and
a nucleic acid molecule comprising a guide RNA sequence that is complementary to a target DNA sequence an aptamer that binds to the aptamer binding protein.

31. A composition comprising:
a polynucleotide comprising a nucleic acid sequence encoding a fusion protein comprising a recombination protein functionally linked to an aptamer binding protein, wherein the recombination protein comprises an amino acid sequence having at least 95% identity to a recombination protein of SEQ ID NO: 166 to SEQ ID NO:491;
a polynucleotide comprising a nucleic acid sequence encoding a Cas protein and at least one peptide aptamer sequence that binds to the aptamer binding protein; and
a nucleic acid molecule comprising a guide RNA sequence that is complementary to a target DNA sequence.

32. A vector comprising:
a polynucleotide comprising a nucleic acid sequence encoding a fusion protein comprising a recombination protein functionally linked to an aptamer binding protein,
a polynucleotide comprising a nucleic acid sequence encoding a Cas protein; and
a nucleic acid molecule comprising a guide RNA sequence that is complementary to a target DNA sequence an aptamer that binds to the aptamer binding protein,
wherein the recombination protein comprises an amino acid sequence having at least 95% identity to a recombination protein of SEQ ID NO: 166 to SEQ ID NO:491.

33. A vector comprising:
a polynucleotide comprising a nucleic acid sequence encoding a fusion protein comprising a recombination protein functionally linked to an aptamer binding protein,
a polynucleotide comprising a nucleic acid sequence encoding a Cas protein and at least one peptide aptamer sequence that binds to the aptamer binding protein; and
a nucleic acid molecule comprising a guide RNA sequence that is complementary to a target DNA sequence,
wherein the recombination protein comprises an amino acid sequence having at at least 95% identity to a recombination protein of SEQ ID NO: 166 to SEQ ID NO:491.

34. A eukaryotic cell comprising the system of claim 1, or the vector of claim 32.

35. A method of altering a target genomic DNA sequence in a cell, comprising introducing the system of claim 1, the composition of claim 30, or the vector of claim 32 into a cell comprising a target genomic DNA sequence.

36. The method of claim 35, wherein the cell is a mammalian cell.

37. The method of claim 35, wherein the cell is a human cell.

38. The method of claim 35, wherein the cell is a stem cell.

39. The method of claim 35, wherein the target genomic DNA sequence encodes a gene product.

40. The method of claim 35, wherein the introducing into a cell comprises administering to a subject.

41. The method of claim 40, wherein the subject is a human.

42. The method of claim 40, wherein the administering comprises in vivo administration.

43. The method of claim 40, wherein the administering comprises transplantation of ex vivo treated cells comprising the system, composition, or vector.

44. (canceled)
